SEQUENCE LISTING (278 sequences)

SEQ ID NO: 1 - protein, 556 aa, Homo sapiens [sequence omitted]

SEQ ID NO: 2 - DNA, 7409 bp, Homo sapiens
  source 1..7409: mol_type = genomic DNA
aggcccctgcctgccccagcatcccctgcgcgaagctgggtgccccggagagtctgaccaccatgccacctcctcgcctcctcttcttcctcctcttcctcacccccatggaagtcaggcccgaggaacctctagtggtgaaggtggaaggtatgtccaaagggcagaaagggaagggattgaggctggaaacttgagttgtggctgggtgtccttggctgagtaacttaccctctctgagcctccattttcttatttgtaaaattcaggaaagggttggaaggactctgccggctcctccactcccagcttttggagtcctctgctctataacctggtgtgaggagtcggggggcttggaggtcccccccacccatgcccacacctctctccctctctctccacagagggagataacgctgtgctgcagtgcctcaaggggacctcagatggccccactcagcagctgacctggtctcgggagtccccgcttaaacccttcttaaaactcagcctggggctgccaggcctgggaatccacatgaggcccctggccatctggcttttcatcttcaacgtctctcaacagatggggggcttctacctgtgccagccggggcccccctctgagaaggcctggcagcctggctggacagtcaatgtggagggcagcggtgagggccgggctggggcaggggcaggaggagagaagggaggccaccatggacagaagaggtccgcggccacaatggagctggagagaggggctggagggattgagggcgaaactcggagctaggtgggcagactcctggggcttcgtggcttcagtatgagctgcttcctgtccctctacctctcactgtcttctctctctctgcgggtctttgtctctatttatctctgtctttgagtctctatctctctccctctcctgggtgtctctgcatttggttctgggtctcttcccaggggagctgttccggtggaatgtttcggacctaggtggcctgggctgtggcctgaagaacaggtcctcagagggccccagctccccttccgggaagctcatgagccccaagctgtatgtgtgggccaaagaccgccctgagatctgggagggagagcctccgtgtctcccaccgagggacagcctgaaccagagcctcagccagggtatggtgatgactggggagatgccgggaagcgggggtccagagacagaggggaggggaaactgaagaggtgaaaccctgaggatcaggctttccttgtcttatctctccctgtcccagacctcaccatggcccctggctccacactctggctgtcctgtggggtaccccctgactctgtgtccaggggccccctctcctggacccatgtgcaccccaaggggcctaagtcattgctgagcctagagctgaaggacgatcgcccggccagagatatgtgggtaatggagacgggtctgttgttgccccgggccacagctcaagacgctggaaagtattattgtcaccgtggcaacctgaccatgtcattccacctggagatcactgctcggccaggtagagtttctctcaactgggaggcatctgtgtgggggtactgggaagaagtggaagccagtcaatcttagattcccccaacccgagggctactcccagcctcaccccaaaccccaacttccacacagaacactgactccaagtctttcttttttttgacagagtctcgctctgttgcctaggctggagtgcagtggtgccatcttgtcttggctcactgcaacctccgcctcccaggttcaagtgattcccctgcctcagcctcctgagtagctgggattacaggtgcccaccaccacgcctggctaattttttttttttttttgagacggagtcttgcactgtcacccaggctggagtgcagtggcacgatctcagctcactgcaacctccaccttccaggttcaagtgattctcctgcctcagcctcccgagtagctgggattaaagcctggctaattttttttgtatttttagtagagatggggtttcattatgttggccaggctggtctcaaactcctgacctcgtgatccacccgcctcggcctcccaaagtgctgggattacagacatgagccacagggccgggccaagcctaattttgtatttttagtagagatggggtttctccctgttggaccaggctggtcttgaactcctgacttcaggtgatctgcctgccttggcctcccaaagtactgggattacaggcataagccaccgcacctggcctagacttcaagtctttcttccctcgcttccaagacactacttttctgggtcttcacctaccattgcttgcgcctgcccaccagcttgggtggagtcttccttcctccccaactcctcactcttggagccctgggccctcttcttatccctgtctgcacactttcctatttgaacttgactctcaatggcttcttgggtcaccatgccttggtgactctattccaggctccatactcagccatctcctgtgccatttgatatcccatggacacctcaggctcaacagatacaaaatcaaactcaatgtcttccccaagtatagtcttcttggtggcccagtgtaagcagagggcaccaccacctgctccctcgcccaggctaagaacctgggcatccttctttttcctcaccccgtccaacaaactggtcacagtgttctgccaattctctctccatgcaatcctatcatgctatcctaactgcaattcacaaacccaaccccaactttcactccaaacttgatccaagcaatgtgctggatcccaactgtaaccttgcaaactcaactctgcccttcactttgaccgtgactatccttaattgcagcaggaaactgatcattatgctcccctcaatccacacattgcctctgagtacagccatggtttgtccacgatttgctcaaagacactgccccatgtcctgtgccagggtctgtgacaatccctgacctcctgggacatggctccttagagagaggagagcctttctcacagcttgggactttgagtctgtgtctttttttttttcttgagacggagttttgctgtggttgcccaggctggagtgcagtgatctcggctcactgaaacctccgcctcccgggttcaaacgattctcctgcctcagcctcccaagtagctgggattacaggcacccaccaccatgcccagctaatttttttgtatttttagtagagatggggtttcaccatgttggccaggctggtctcgaactcctgacctcaggtgatccacccgcctttgcctcccaaagtgctgggattacaggcgtcaaccaccgcgcccggccgagtctgtgtcttgcctctgtgcctcagacttgcggttccttgagatctcaggattgggacgtaagatgccagcctggggtcctcgtctcatagccccttccccctagtactatggcactggctgctgaggactggtggctggaaggtctcagctgtgactttggcttatctgatcttctgcctgtgttcccttgtgggcattcttcatcttcaaagaggtgagtcatgtccccagtgggtctgtccaaaccctactccatcttccccaggataagccggctctggccagtctgacaaccatctttctttcctcccatccctcccttcaagaccccagaatcctgttctccccagtcttcctctagcctccctcaaacttcccaagcctcttgcaattttttttttttttttgagacagggtctcattctgtcaccccagctggagtgcagtggcacaatctgagctcactgtaacctctgcctcccaggcttaagtgattcttgtgcttcagcctcccgagtacctgggactacaagtgtatgccaccacacccggccaattttttatatttttagtagagacgaggtttcaccatgttggccagactggtctcgaactcttgacctcaaatgatccgcccacctcggcctcccaaagtgctgggattacaggcacgagccaccgcgcccgtccgcctcgcaatttgaactcctgtctcctttgttgaaccaagtgacctccccagcacctggccccacaaatcctcaccctgccaagcagcccctcctctgatcacgccctttaactcccaccagccctggtcctgaggaggaaaagaaagcgaatgactgaccccaccaggaggtaatgcaaccagtgcaccccgcggtaacaccctccaccttcactttatgccttgcacttactgtttcctctgcccaggggttctttgctccgtctctactgtttcaaatactgcccaacctcaaagcccagctccaaagctacctcctctgtgaagaactccttggaaatgatcatctcagactcctctattggctgtcccagcacaagtgatcacgtttaacttctgaaggcctggacagaatcttgagtgggtccgccattccattccaagtcggccctcaccgtgcacttcctcttctcccgccagattcttcaaagtgacgcctcccccaggaagcgggccccagaaccagtacgggaacgtgctgtctctccccacacccacctcaggcctcggtaagaggcaccgcccctccagcctatagctccgccccagatccggggctccacccccactctcctcatccctccaatccgctgtgcgccaagccttctggagctcggaactccgcccccggggcggggagtcccgcccagctatgagccccgcctctagaaccagaccccgcctccagggctcagagccacgcccccaggacccagagcctgaagtcgtaatcaagagcagaacttcgccccagaactgaaggcctcggccctagatttagattccgccccagggttcaaggccgggttcctagacccagagtccattcgcagagcccaaaacatcctcttcccgtgccccgccgcgcggacccttagccttgaccgcccccatctcttctgaccccgtcttacaatgcccctctcaccaggacgcgcccagcgttgggccgcaggcctggggggcactgccccgtcttatggaaacccgagcagcgacgtccaggcggatggagccttggggtcccggagcccgccgggagtgggtgaatgactgggagagggaagggtcgttccccacatggagggggttggagcggtctgtggcccgaatagtggactgggccctggaggagagggggcatgactcggttccccatccccatccccaaacccccaggcccagaagaagaggaaggggagggctatgaggaacctgacagtgaggaggactccgagttctatgagaacgactccaaccttgggcaggaccagctctcccagggtaaggctgccctcccccgtggccccccacctctgcggtggcctgtggactcccatggacacccctccttctccaccagatggcagcggctacgagaaccctgaggatgagcccctgggtcctgaggatgaagactccttctccaacggtaacttggggcctttgtgggacctcagagacttaggtgtaattgcagcgctgtgacactcctagaaggggtccctggagttctctctcttctgccacagctgagtcttatgagaacgaggatgaagagctgacccagccggtcgccaggacaatgggtgtgtgtgaggatggcaacagtccaggggggaggcggaggacacctggaggccaggaggaatagtaacctccctcttccctttccagacttcctgagccctcatgggtcagcctgggaccccagccgggaagcaacctccctgggtgagagatgctttcaatcagactgccttgcccagcttgggtgacctggcctcagctctgacaccagatccaactttgacctgaccctgaccccaaacccgaacccaatcctgtgactcctctcacctcaacactgagccccatcccccatcctgagccccatcccccatcctgacccccaatatttaccccctccctaactgtgaatatcaacaccgatcccaatgcagtatcagcctggacttgatctccacctcacctcagccccagtgcagacctcaacttggaccccagcttactctgcagcttcttcatgactctgactccgactccctccagtttcttctttttctttttcttttttttgagacggagtctccctctgttgcccaggctggagtgcagttgccacctctgcctcctaggttcaagcgattctcatgcctcagcctcctgagtagctgggattatagacgtttgccaccacacctggctaatttttgtattttcagtagagacagggtttcgccatgttggccagactggtctccaactcctggcctctagtgatctgcccgccttggcttcccaaagtgctgggattacaggcatgagccaccacgcccagcccagttctgttcttgaccccttccttagccataatctaacccatatctaaccctgaccctacagctaactggggccccaaactcaatgctaaccaaatcaccccttcccagcacagcatgggtaatgctcctcaccttcctctgcccctcagtcttcctccttaccgtaggctgtacttcccatgccctagcctccaattctccatcccccgcccaagcagggtcccagtcctatgaggatatgagaggaatcctgtatgcagccccccagctccgctccattcggggccagcctggacccaatcatgaggaaggtgggtgcttctgccgctgtcccctgctgtcccctgggctgactttgccttccagcctacttccagtgccacccatgttctcctcctccctggtcctatccagatgcagactcttatgagaacatggataatcccgatgggccagacccagcctggggaggagggggccgcatgggcacctggagcaccaggtgatcctcaggtggccaggtgagctgggactgcccctagggaaagcggggagggagggagataggcacggatggcagtggctgctggctttcagggagggagagggaacagggttcctagggcctggtgggcagggggaggactgctggacccctccccatcaccgtttcttctgcatagcctggatctcctcaagtccccaagattcacacctgactctgaaatctgaagacctcgagcagatgatgccaacctctggagcaatgttgcttaggatgtgtgcatgtgtgtaagtgtgtgtgtgtgtgtgtgtgtgtatacatgccagtgacacttccagtcccctttgtattccttaaataaactcaatgagctcttccaatccta

SEQ ID NO: 3 - protein, 557 aa, Macaca fascicularis
MPPPCLLFFLLFLTPMEVRPQEPLVVKVEEGDNAVLQCLEGTSDGPTQQLVWCRDSPFEPFLNLSLGLPGMGIRMGPLGIWLLIFNVSNQTGGFYLCQPGLPSEKAWQPGWTVSVEGSGELFRWNVSDLGGLGCGLKNRSSEGPSSPSGKLNSSQLYVWAKDRPEMWEGEPVCGPPRDSLNQSLSQDLTMAPGSTLWLSCGVPPDSVSRGPLSWTHVRPKGPKSSLLSLELKDDRPDRDMWVVDTGLLLTRATAQDAGKYYCHRGNWTKSFYLEITARPALWHWLLRIGGWKVPAVTLTYLIFCLCSLVGILQLQRALVLRRKRKRMTDPTRRFFKVTPPPGSGPQNQYGNVLSLPTPTSGLGRAQRWAAGLGGTAPSYGNPSSDVQVDGAVGSRSPPGAGPEEEEGEGYEEPDSEEGSEFYENDSNFGQDQLSQDGSGYENPEDEPLGPEDEDSFSNAESYENEDEELTQPVARTMDFLSPHGSAWDPSREATSLGSQSYEDMRGLLYAAPQLRTIRGQPGPNHEEDADSYENMDNPDGPDPAWGGGGRMGTWSAR

SEQ ID NO: 4 - DNA, 8315 bp, Macaca fascicularis
  source 1..8315: mol_type = genomic DNA
gggtgccccggggagtctgaccaccatgccacctccttgcctcctcttcttcctcctcttcctcacccccatggaagtcaggccccaggaacctctagtggtgaaggtagaaggtatgtccaaagggcagaaagggaagggatggaggctggaaacttgggttgtggctgggtgtccttggctgagtaacttaccctctctgagcctccattttcttatttgtgaaattcagggaggggttggaaggactctgccggctcctccactcccagtttttggagtcctctgatctataacccggtgtgagcagtgggggggcttggaggtccccccgtcatgcccacacctctctccctctctctccacagagggagataacgctgtgctgcagtgcctcgaggggacctcagatggccccactcagcagctggtctggtgtcgggactccccgtttgaacccttcttaaatctcagcctggggctgccaggcatgggaatccgcatggggcccctgggcatctggcttttaatcttcaacgtctctaaccagacggggggcttctacctatgccagccggggctcccctctgagaaggcctggcagcctggctggacagtcagtgtggagggcagcggtgagggccgggctggccgggctagggcaggggcaggaggagagaagggaggccaccatggacagaagaagtccgcggccacaatggagctggagagaggggctggagggattgagggcgaaactcggagctaggtgggcagactcctggggcttcgtggcttcagcatgagctgcttcctgtccctccacctctcactgccttctctctctgtctctctctctctgagtctttgtctctatttatctctgcctttgagtctctctctctctccctctcctgggtgtctctgcatttggttctgggtctcttcccaggggagctgttccggtggaatgtttcggacctaggtggcctgggctgtggcctgaagaacaggtcctcagagggccccagctccccttctgggaaactcaacagctcccaactgtatgtgtgggccaaagacagacctgagatgtgggagggagagcctgtgtgtggcccaccgagggacagcctgaaccagagcctgagccagggtaaggtgatgattggggagatgccaggaagtgggggtccagagacggaggggaggggaaactgaagaggtgtgagaccctgaggatcaggctttcctcgtcttatctctccctgccccagacctcaccatggccccgggctccacactctggctgtcctgtggggtaccccctgactctgtgtccaggggccccctctcctggacccatgtgcgccccaaggggcctaagtcctcattgttgagcctagagctgaaggacgatcgcccagacagagatatgtgggtagtggacacgggtctgttgttgacccgggccacagctcaagacgctgggaagtattattgtcaccgtggcaactggaccaagtcattctacctggagatcactgctcggccaggtagagtttctctcaactgggaggcatctgtgtggggtgtactggcaagaagtgaaagccagtcaatcttaggttcccccaacctgagggctactcccagcctcaccccaaaccccaacttccacacagaacattgactccaagtctttttttttttttttgaccgagtcttgctctgtcgcctaggctggagtgcagtggtgccatctcgtcttggctcactgcaacctccgcctcccaggttcaagcgattctcctgcctcagcctcctgaatagctgggattacaggtgcccaccaccatacctggctaattttttttttttgagaggagtctttcactgtcacccaggctggagtgcaatggtgcaatctcagctcactgcaactttcgccttccaagttcaagtgattctcctgcctcagcctcccgagtagctgggattacaggcgctcgccaccatgcctggctaatttttttgttttttttttgttttttttttgtttgagacggagtctcactctgtcccccttgctggagtgcagtggccggatctcagctcactgcaagctccgcctcccgggttcaggcaattctcctgcctcagcctcccgagtagctgggaatacaggcgcccgccacctcgcccagctagtttttttgtattttttagtagagacggggtttcactgtgttagccaggatgttctcgatctcctgacctcgtgatccgcccgtctcggcctcccaaagtgctgggattacaggcttgagccaccgcgcccagcctgtattttttttttgagatggagtcttgctctgtcgcccaggctagagtgcagtggtcggatctcagctcactgcaagctccgcctcctgggtttacgccattctcctgcctcagcctcccgagtagctgggactacaggcgcccgccacctcgcccggctagttttttgtattttttagtagagacggtgtttcaccgtgttagccaggatggtctcgatctcctgacctcatgatctgcccatctcggcctcctgaagtgctgggattacaggcttgagccactgtgcccggccaattttttgtatttttagtagagacggggtttcattatgttggccaggttggtctcgaactcctgacctcgtgatccacccgtctcggcctcccaaagtgctgggattacaggcatgagccacagggcagggtcaggcctaatttttgtatttttagtagagatggggtttctccatgttggaccaggctggtcttgaactcctaacttcaggtgatccgcctgccttggcctcccaaagtactgggattacaggcgtgagccaccacacctggcctagacttcaagtctttcttccctcgcttccaatacactacttttctgggtcttcacctaccattgcttgtgcctgcccaccagcttgggtggagtcttccttcctctccaactcctcactcttggagccctgggccctcttcttatccctgtctgcacgctttcccatttgaccttgactcccaatggctttttgggtcactatgccttggtgactctattccaggctccatattcagccgtcccctgtgccatttgatgtcctatggacacctcaggctcaacagatacaaaatcaaactcaatgtcttccccaagtatagtcttcttggtggccctgtgcaagcagagggcaccaccacctgctcccttgcccaggctaagaacctgggcatccttccttttcctcaccctgtccagcaaactggtcacagtgttctgccagttctctctccttcatacaatcctatcatgctatcctaaccgcaatttacaaactcaaccccaactttcactcaaaacttgatccaagcaatgtgctggatcccaactgtaaccttgcaaactcaactctgcccttcactttgactgtgactgtccttaattgcagcaggaaactgatcattatgctctcctcaatccacacattgcctctaaatacagccatggtttgcctgtgatttactcaaaagacactgccccatgtcatgtgccaggctctgtgacaatccctgacctcctgggacatgactccttagagagaggagagcctttctcacagtttgggactttgagtctgtctttttttttttctcttgagacagagtttcgctcttgttgcccaggctggagtgcagtgatctctgctcactgaaatctccactcccgggttcaagcgatactcctgcctcagcctcccaagtagctgggattacaggcgcccgccatcatacctagctaatttttttgtatttttagtagagacagggtttcaccatgttggccaggctggtctcgaactcctgacctcaggtgatccacccgccttggcctcccaaagtgctgggattacaggcgtggaccactgcacctcgccaagtctgtgtcttgcctctgtgccccagacttgcggttctttgagatctcgggattggaacgtaagatgccagcctggggtcctcatctcatagccccttccccctagcactatggcactggctgctgaggattggtggctggaaggtcccagctgtgactttgacttatctgatcttctgcctgtgttcccttgtgggcattcttcaacttcaaagaggtgagtcatgtccccagtgggtctgtccaaaccctacaccatcttccccaggataagccggctttggccagtctgacaaccatctttccttcctcccatccctccctgcaagaccccagaatcctgttccccccagtcttcctctagcctccctcaaacttccgaagcctcttgcatttttttttttttttttttgagacagggtctcattctgtcaccccagctggagtgcagtggcacaatctgagctcactgtaacctctgcctcccaggctcaagtgattcttgggcctcagcctcctgagttgctgggactacaagtgtgtgccaccgcgcccagctgattttttatatttttagtagagacagggtttcgccacattgcccacgctggtcttgaactcctgagcttaggcaattcgctggcctcatcttcctaaagtgctgggaatataggcgtgagctacagcgcctggctccctcaaacttttttttttttttttttgagatggagtttttgctctgttgcccatgttggagtgcaatggcatgatctctgctcactgcaacctccacctcctgggttcaagcgattctcttgcctcagcctccctagtagctaggatcacaggcgcccaccaccacgtccggctaatttttatatttttagtaaagacgaagtttcaccatgttggccaggctggtctcgaactcttgacttcaaatgatccgcccacctcggcctcccaaagtgctggggttacaggcactagccaccgcgcccgtcccccttgcaatttgaactcctgtcttctctgttgaaccaagtgacctccgcagtacctggccccacaaatcctcaccctgccaagcagcgcctcctctgatcactccctttaactcccaccagccctggtcctgagaaggaaaagaaagcgaatgactgaccccaccaggaggtaatgcaaccagtgcacccctcggtaacaccctccaccttcactttatgccttgcacttactgtttcctctgcccaggggttctttgctccgtctctactgtttcaaatactgcccaacctcaaagcccagctccaaagctacctcctctgtgaagacctccttggaaatgatcatctcagactcctctattggctgtcccagcacaagtgatcacgtttaatctctgaaggcctggacagaatcttgagtgggtccgccattccattccaagtcggccctcaccgtgcacttgcccttctcccgccaggttcttcaaagtgacgcctcccccaggaagcgggccccagaaccagtatgggaacgtgctatctctccccacacccacctcaggcctcggtaagaggcaccgcccctccagcctatagctccgccccagatccagggctccacccacactgtcctcatccctccaatccgctgtgcgccatgccttctggagctcggaactccgccaccggggagggtagtcccgcccatctatgagccccacccctagaacaatgccccgcctccagggctcagagccacgcccccaggacccagagcctgaagtccaaatcagaagcagaacctcgccccagaactgaaggccttggccctagatttagatcccgccctatggttcaaggccaggtgcctagactcagagtccattcgcagagcccaaagcattctctccccgtgccccgccgcgcggacccttagccttgaccgcccccatctcttctgaccccgtcttacaactcccgcccctctcgccaggacgcgcccagcgttgggccgcaggcctggggggcaccgccccgtcttatggaaatccaagcagcgacgtccaggtggatggagccgtggggtcccggagccctccgggagcgggtgaatgattgggggagggaagggtcgttccccacatagagggggttggagaggtctgtggcccaaatagtggactggggcctggaggagagggggcgtaactcggttccccatccccatcccgaaacccctaggcccagaagaagaggaaggggagggctatgaggagcccgacagtgaggagggctccgagttctatgagaacgactccaactttgggcaggaccagctctcccagggtaaggctgccctcctccgtggtcccccgcctctctgcggtggcctgtggactcccatggacacccctccttctccaccagatggcagcggctacgagaaccctgaggacgagcccctgggtcctgaggatgaagactctttctctaacggtaacttggggcctttgtgggacctcagagacttaggtgtaattgcagcgctgtgacacccctagaaggggtccctggagttctctctcttctgccacagctgagtcttacgagaacgaggatgaagagctgacccagccggtcgccaggacaatgggtgtgtgtgaggatggcaacagtctaggggggaggtggaggacgccaggaggccaggaggaatagtaacctccctcttccctttccagacttcctgagcccccatgggtcagcctgggaccccagccgggaagcaacttccctgggtgagagatactttcgaccagactgccttacccagcttgggtgacctggcctgagctctgacaccagacccaactttgacctgaccttgaccccaaacccgaacccaatactgaccttggatttgactcctctcacctcagtactaagccccatcccccatcctgacccccaatatttaccccctccctaactatgaatatcaacaccgatcctgatccagtatcaagctgcacttgacctccacctcacctcagcccctcagtgcagacctcaacttggaccccagctcactctgcagcttcttcatgactctgactccttccagttttttcttttttctcttttttttgaattggagtctcgctctgttgcccaggctgtagtgcagttgccacctctgcctcccaggttcaagcgattctcatgcctcagcctcctgagtagctgggattacagatgtttgccaccacacctggctaatttttgtatcttcagtagagacagggtttcaccatgttggccagactggtctcagactccttacctctaatgatctgcccgcctcggcctcccgaagtgccgggattacaggtgtgagccaccatgcccagcccagttctgttcgtgaccccttctctagccataatctaacccatatctagccgtgactctacagctaaccggggtcccaaacccgatgctgaccgaatcaccccttcccagcacagcataggtgatgcccctcaccttcctctgcccctcattcttcctccttaccataggctgtacttcccgtgccctagcctccaattctccatcccccgcccaagcagggtcccagtcctatgaggatatgagagggctcctgtatgcagccccccagctccgcaccattcggggccagcctggacccaatcatgaggaaggtgggtgcttctgccgctgtcccctgctgtcccctgggctgactttgcctcccagcctacttccagtgccacccgtgttctcctccctggtcctatccagatgcagactcttacgagaacatggataatcccgatgggccagacccagcctggggaggaggcggccgcatgggcacctggagtgccaggtgatcctcaggtggccaggtgagctgggactgcccctagggaaaggggggagggagggagataggcacgaatgacagtggctgctggctctcagggagggagagggaacagggctcctagggcctggtgggcaaggggagggctgctggacccctccccatcactgtttcttcttcatagcctggatctcttcaagtccccaagatccacacctgactctgaaatctgaagacctcgagcagatgatgccaacctctggagcaatgttgcttaggatgtgtgcatgtgtgtaagtgtgtgtgtgtgggtatacatgccagtgacactttcagtcccttttgtattccttaaataaactcaatgagctcttccaatccta

SEQ ID NO: 5 - protein, 186 aa, Homo sapiens
QDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 6 - DNA, 713 bp, Homo sapiens
  source 1..713: mol_type = genomic DNA
ttcacccccaggagcccatccggaaaggccagcgggacctgtattctggcctgaatcagagacgcatctgacctcttggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttggtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaaccccccctcccactgtaggccactggatggtcatttggcatctccgtatatgtgctctggctcctcagctgagagagaaaaaaataaactgtatttggctgcaaagagtt

SEQ ID NO: 7 - protein, 198 aa, Homo sapiens
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 8 - DNA, 597 bp, Macaca fascicularis
  source 1..597: mol_type = genomic DNA
atgcagtcgggcactcgctggagagttctgggcctctgcctcttatcaattggcgtttgggggcaagatggtaatgaagaaatgggtagtattacacagacaccatatcaagtctccatctctggaaccacagtaatactgacatgctctcagcatcttggatctgaagcacaatggcaacacaatggtaaaaacaaagaagattctggggatcggctgtttctgccggaattttcagaaatggagcaaagtggttattatgtctgctaccccagaggaagcaatccagaggacgcgagccatcatctctacctgaaggcaagagtgtgtgagaactgcatggagatggatgtgatggcggtggccacaattgtcatagtggacatctgcatcactctgggcttgctgctgctggtttactactggagcaagaatagaaaggccaaggccaagcctgtgacacgaggagcaggtgctggcggcaggcaaaggggacaaaacaaggagaggccaccacctgttcccaacccagactatgagcccatccggaaaggccagcaggatctgtattctggcctgaatcagagacgcatctga

SEQ ID NO: 9 - protein, 5 aa, Homo sapiens
DYYWS

SEQ ID NO: 10 - protein, 16 aa, Homo sapiens
EITHSGSTNYNPSLKS

SEQ ID NO: 11 - protein, 14 aa, Homo sapiens
GGLDIFTGPYYFDS

SEQ ID NO: 12 - protein, 122 aa, Homo sapiens
QVQLQQWGAGLLKPSETLSLTCAVYGGSFSDYYWSWVRQPPGKCLEWIGEITHSGSTNYNPSLKSRVTISVDTSKNQFSLKLSSVTAADTAVYYCARGGLDIFTGPYYFDSWGQGTLVTVSS

SEQ ID NO: 13 - DNA, 366 bp, Homo sapiens
  source 1..366: mol_type = genomic DNA
caagtgcagcttcagcagtggggcgccggtctgctgaagccttccgaaaccctctcgctgacttgcgccgtctacggcgggagcttcagcgattactactggagctgggttcgccagccgccgggaaagtgtctggagtggatcggagagattacgcattccggatccaccaactacaatcctagcctgaagtcaagagtgaccatttctgtggacacctccaagaaccagttttccctgaaactgtcctccgtgactgccgcggataccgcggtgtactactgcgcacgcgggggactggacatcttcaccggaccgtattacttcgactcctggggacagggaactctggtcaccgtcagctcc

SEQ ID NO: 14 - protein, 11 aa, Homo sapiens
RASQEIRKDLA

SEQ ID NO: 15 - protein, 7 aa, Homo sapiens
GASSLRS

SEQ ID NO: 16 - protein, 9 aa, Homo sapiens
QQDYNYPPT

SEQ ID NO: 17 - protein, 107 aa, Homo sapiens
DIQMTQSPSSLSASVGDRVTITCRASQEIRKDLAWYQQKPGKAPKLLIYGASSLRSGVPSRFSGSGSGTDFTLTISSLQPEDFATYYCQQDYNYPPTFGCGTKVEIK

SEQ ID NO: 18 - DNA, 321 bp, Homo sapiens
  source 1..321: mol_type = genomic DNA
gacatccagatgacccaatccccttcctcgctgagcgcctccgtcggggacagagtcaccatcacatgccgcgcaagccaagagatccggaaggacctggcctggtaccagcagaaacccggaaaggctccgaagctgctgatctacggagcttcctctctccggtcgggcgtgcctagccggttcagcggtagcggatccggaactgatttcaccctgaccatttcctcgctgcaacccgaggacttcgccacctactactgccaacaagactacaactatccacctacgtttgggtgcggcaccaaggtcgaaatcaag

SEQ ID NO: 19 - protein, 5 aa, Homo sapiens
RYGIH

SEQ ID NO: 20 - protein, 17 aa, Homo sapiens
AIWYDASKTYYADSVKG

SEQ ID NO: 21 - protein, 16 aa, Homo sapiens
SNYDILTRYYQYGLDV

SEQ ID NO: 22 - protein, 125 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYQYGLDVWGQGTTVTVSS

SEQ ID NO: 23 - DNA, 375 bp, Homo sapiens
  source 1..375: mol_type = genomic DNA
caagtgcagctcgtcgaatccggcggcggagtggtgcagcctggcggatcgctgcggctgtcatgcgcggcttccggcttcaccttctcgcgctacgggatccactgggtcagacaggcccccggaaagtgcctcgaatgggtggccgccatttggtacgacgcatccaagacctattacgcggatagcgtgaagggccgcttcactatctcccgggacaacagcaagaacactctgtacctccaaatgaatagcctgagggctgaggacaccgccgtgtactactgcgcccgctccaactacgacattctgacccggtactatcagtacgggcttgacgtctggggacaggggaccaccgtgactgtgtcatcc

SEQ ID NO: 24 - protein, 11 aa, Homo sapiens
RASQGISSALA

SEQ ID NO: 25 - protein, 7 aa, Homo sapiens
DASSLES

SEQ ID NO: 26 - protein, 9 aa, Homo sapiens
QQFKSYPRT

SEQ ID NO: 27 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFKSYPRTFGCGTKVEIK

SEQ ID NO: 28 - DNA, 321 bp, Homo sapiens
  source 1..321: mol_type = genomic DNA
gctatccaactcacccaatcaccaagctccctgtccgcgtccgtgggcgatagagtgaccatcacttgccgggccagccagggcatttcttcggccctggcctggtaccagcagaagcctggaaaagccccgaagctgctgatctacgacgcaagctcattggagagcggagtgccgtcccgcttctccggatccggttcggggaccgacttcactctgaccatttcctccctgcaaccggaagatttcgcgacatactactgtcaacaattcaagtcgtacccccggacgttcggatgcgggaccaaagtcgagatcaag

SEQ ID NO: 29 - protein, 495 aa, Homo sapiens
PEEPLVVKVEEGDNAVLQCLKGTSDGPTQQLTWSRESPLKPFLKLSLGLPGLGIHMRPLAIWLFIFNVSQQMGGFYLCQPGPPSEKAWQPGWTVNVEGSGELFRWNVSDLGGLGCGLKNRSSEGPSSPSGKLMSPKLYVWAKDRPEIWEGEPPCLPPRDSLNQSLSQDLTMAPGSTLWLSCGVPPDSVSRGPLSWTHVHPKGPKSLLSLELKDDRPARDMWVMETGLLLPRATAQDAGKYYCHRGNLTMSFHLEITARPVLWHWLLRTGGWKGGGGSGGGGSGSAAADEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPGTSHHHHHHHHHHHH

SEQ ID NO: 30 - protein, 120 aa, Homo sapiens
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 31 - protein, 107 aa, Homo sapiens
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 32 - protein, 122 aa, Homo sapiens
QVQLQQWGAGLLKPSETLSLTCAVYGGSFSDYYWSWVRQPPGKGLEWIGEITHSGSTNYNPSLKSRVTISVDTSKNQFSLKLSSVTAADTAVYYCARGGLDIFTGPYYFDSWGQGALVTVSS

SEQ ID NO: 33 - protein, 107 aa, Homo sapiens
EIVLTQSPSSLSASVGDRVTITCRASQEIRKDLAWYQQKPGKAPKVLIYGASSLRSGVPSSFSGSGSGTDFTLTISSLQPEDFATYYCQQDYNYPPTFGQGTKVEIK

SEQ ID NO: 34 - protein, 121 aa, Homo sapiens
QVQLVQSGAEVKKPGESLKISCKGSGYSFTSYWIGWVRQMPGKGLELMGIIYPGDSDTRYSPSFQGQVTISADKSISTAYLQWSSLKASDTAMYYCARGGTTIFGVVFEYWGQGTLVTVSS

SEQ ID NO: 35 - protein, 107 aa, Homo sapiens
EIVLTQSPSSLSTSVGDRVTITCRASQSINNYLNWYQQKPGKAPKLLIYGASSLQSGVPSRFSGSGSGTDFTLTISSLQPEDFATYYCQQTYSTPWTFGQGTKVEIK

SEQ ID NO: 36 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYQYGINVWGQGTTVTVSS

SEQ ID NO: 37 - protein, 107 aa, Homo sapiens
AIQLTQSPSSLSASVGDRVTITCRASQGISSALAWYQQKPGKAPKLLIYDASSLENGVPSRFSGSGSGTDFTLTISSLQPEDFATYYCQQFKSYPRTFGQGTKVEIK

SEQ ID NO: 38 - protein, 125 aa, Homo sapiens
QVQLVESGGGVVQPGRSLRLSCAASGFTFSNYGIHWVRQAPGKGLEWVAAIWYDASKKYYADSVKGRFTISRDNSKNTLYLQMNSLRAEDTAVYYCARSNYDILTRYYQYGINVWGQGTTVTVSS

SEQ ID NO: 39 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFKSYPRTFGQGTKVEIK

SEQ ID NO: 40 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSS

SEQ ID NO: 41 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLYLQMNSLRAEDTAVYYCARSNYDILTRYYQYGINVWGQGTTVTVSS

SEQ ID NO: 42 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTRYYSHGINVWGQGTTVTVSS

SEQ ID NO: 43 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPRTFGQGTKVEIK

SEQ ID NO: 44 - protein, 122 aa, Homo sapiens
EVQLVQSGAEVKKPGESLKISCKGSGYSFTSYWIGWVRQMPGKGLEWMGIIYPGDSDTRYSPSFQGQVTISADKSLSTAYLQWSGLKASDTAMYYCARQSIMGVIYVAFDVWGQGTMVTVSS

SEQ ID NO: 45 - protein, 107 aa, Homo sapiens
EIVLTQSPSSLSASVGDRVTITCRASQSISSYLNWYQQKPGKAPKLLIYAASSLQSGVPSRFSGSGSGTDFTLTITSLRPEDFATYYCQQSYSNPWTFGQGTKVEIK

SEQ ID NO: 46 - protein, 121 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGGATIFGVVFDYWGQGTLVTVSS

SEQ ID NO: 47 - protein, 107 aa, Homo sapiens
EIVLTQSPSSLSASVGDRVTITCRASQSINSYLSWFQQKPGKAPKLLIYAASSLQSGVPSRFSGSGSGTDFTLTISSLQPEDFATYYCQQSYSTPPTFGQGTKVEIK

SEQ ID NO: 48 - protein, 122 aa, Homo sapiens
QVQLQESGPGLVKPSETLSLTCTVSGGSISSYYWSWIRQPPGKGLEWIGYIYYSGSTKYNPSLKSRVTISVDTSKNQFSLQLSSVTAADTAVYYCATGLAAAGDYYYGMDVWGQGTTVTVSS

SEQ ID NO: 49 - protein, 107 aa, Homo sapiens
EIVLTQSPSSLSASVGDRVTITCRASQGISSALAWYQQKPGKAPKLLIYDASSLESGVPSRFSGGGSGTDFTLTISSLQPEDFASYYCQQFNSYPLTFGGGTKVEIK

SEQ ID NO: 50 - protein, 122 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQSIVATITWAFDIWGQGTMVTVSS

SEQ ID NO: 51 - protein, 107 aa, Homo sapiens
EIVLTQSPSFLSASVGDRVTITCRASQGISSYLAWYQQKPGKAPKLLIYAASTLQSGVPSRFSGSGSGTEFTLTVSSLQPEDFATYYCQQLNSYPYTFGQGTKLEIK

SEQ ID NO: 52 - protein, 117 aa, Homo sapiens
QVQLQESGPGLVKPSETLSLTCTVSGGSISSGSYNWGWIRQPPGKGLEWIGSIYYSGSTYYNPSLKSRVTISGDTSKNQFSLKLSSLTAADTAVYYCARHTGGFDYWGQGTLVTVSS

SEQ ID NO: 53 - protein, 107 aa, Homo sapiens
EIVLTQSPATLSVSPGERATLSCRASQSVSSNLAWYQQKPGQAPRLLIYGASIRATGIPARFSGSGSGTEFTLTISSLQSEDFAVYYCQQYNNWPLTFGGGTKVEIK

SEQ ID NO: 54 - protein, 126 aa, Homo sapiens
QVQLVQSGAEVKKPGESLRISCKGSGYSFSSYWIAWVRQMPGKGLEWMGIIYPGDSDTRYSPSFQGQVTISADKSISTAYLQWSSLKASDTAMYYCARHGGYNYGFTDYYYGMDVWGQGTTVTVSS

SEQ ID NO: 55 - protein, 112 aa, Homo sapiens
DIVMTQTPLSSPVTLGQPASISCRSSQRLVHSDGDTYLSWLQQRPGQPPRLLIYKISNRFSGVPDRFSGSGAGTDFTLKISRVEAEDVGVYYCMQATQFPYTFGQGTKLEIK

SEQ ID NO: 56 - protein, 122 aa, Homo sapiens
EVQLVQSGAEVKKPGESLKISCKGSGYSFTSYWIGWVRQMPGKGLEWMGIIYPGDSDTRYSPSFQGQVAISADKSISTAYLQWSSLKASDTAMYYCARRSIAGTTSWSFDLWGRGTLVTVSS

SEQ ID NO: 57 - protein, 107 aa, Homo sapiens
EIVLTQSPSSLSASVGDRVTITCRTSQDIRNDLGWYQQKPGNAPKLLIYAASSLQSGVPSRFSGSGSGTDFTLTISSLQPEDFATYYCLQDYNYPLTFGGGTKVEIK

SEQ ID NO: 58 - protein, 117 aa, Homo sapiens
QVQLQESGPGLVKPSETLSLTCTVSGGSFSSYYWSWIRQPPGKGLEWIGYIYYSGRTNYNPSLKSRVTISIDTSKNQVSLKLSSVTAADTAVYYCASHHWGSALDYWGQGTLVTVSS

SEQ ID NO: 59 - protein, 112 aa, Homo sapiens
EIVLTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPWTFGQGTKVEIK

SEQ ID NO: 60 - protein, 122 aa, Homo sapiens
QVQLQQWGAGLLKPSETLSLTCAVYGGSFSDYYWSWVRQPPGKGLEWIGEITHSGSTNYNPSLKSRVTISVDTSKNQFSLKLSSVTAADTAVYYCARGGLDIFTGPYYFDSWGQGTLVTVSS

SEQ ID NO: 61 - protein, 107 aa, Homo sapiens
DIQMTQSPSSLSASVGDRVTITCRASQEIRKDLAWYQQKPGKAPKLLIYGASSLRSGVPSRFSGSGSGTDFTLTISSLQPEDFATYYCQQDYNYPPTFGQGTKVEIK

SEQ ID NO: 62 - protein, 122 aa, Homo sapiens
QVQLQQWGAGLLKPSETLSLTCAVYGGSFSDYYWSWVRQPPGKCLEWIGEITHSGSTNYNPSLKSRVTISVDTSKNQFSLKLSSVTAADTAVYYCARGGLDIFTGPYYFDSWGQGALVTVSS

SEQ ID NO: 63 - protein, 107 aa, Homo sapiens
EIVLTQSPSSLSASVGDRVTITCRASQEIRKDLAWYQQKPGKAPKVLIYGASSLRSGVPSSFSGSGSGTDFTLTISSLQPEDFATYYCQQDYNYPPTFGCGTKVEIK

SEQ ID NO: 64 - protein, 125 aa, Homo sapiens
QVQLVESGGGVVQPGRSLRLSCAASGFTFSRYGIHWVRQAPGKGLEWVAAIWYDSSKKYYADSVKGRFTISRDNSKNTLDLQMNSLRAEDTAVYYCARSNYDILTRYYQYGINVWGQGTTVTVSS

SEQ ID NO: 65 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLDLQMNSLRAEDTAVYYCARSNYDILTRYYQYGINVWGQGTTVTVSS

SEQ ID NO: 66 - protein, 125 aa, Homo sapiens
QVQLVESGGGVVQPGRSLRLSCAASGFTFSRYGIHWVRQAPGKGLEWVAAIWYDGSHKYYADSVKGRFTISRDNSKNTLDLQMNSLRAEDTAVYYCARSNYDILTRYYQYGINVWGQGTTVTVSS

SEQ ID NO: 67 - protein, 125 aa, Homo sapiens
QVQLVESGGGVVQPGRSLRLSCAASGFTFSRYGIHWVRQAPGKGLEWVAAIWYDGSNKYYADSVKGRFTISRDNSKNTLDLQMNSLRAEDTAVYYCARSNYDILTRYYQYGINVWGQGTTVTVSS

SEQ ID NO: 68 - protein, 125 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTRYYQYGLDVWGQGTTVTVSS

SEQ ID NO: 69 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRHYQYGINVWGQGTTVTVSS

SEQ ID NO: 70 - protein, 125 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTRYYSHGINVWGQGTTVTVSS

SEQ ID NO: 71 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTGHYQYGINVWGQGTTVTVSS

SEQ ID NO: 72 - protein, 125 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYSHGINVWGQGTTVTVSS

SEQ ID NO: 73 - protein, 107 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPRTFGCGTKVEIK

SEQ ID NO: 74 - protein, 125 aa, Homo sapiens
QVQLVESGGGVVQPGGSLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSS

SEQ ID NO: 75 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTGYYQYGINVWGQGTTVTVSS

SEQ ID NO: 76 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYQHGINVWGQGTTVTVSS

SEQ ID NO: 77 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYSHGINVWGQGTTVTVSS

SEQ ID NO: 78 - protein, 125 aa, Homo sapiens
QVQLVESGGGVVQPGESLRLSCAASGFTFSRYGIHWVRQAPGKGLEWVAAIWYDASKDYYADSVKGRFTISRDNSKNTLYLQMNSLRAEDTAVYYCARSNYDILTRYYQYGLDVWGQGTTVTVSS

SEQ ID NO: 79 - protein, 125 aa, Homo sapiens
QVQLVESGGGVVQPGRSLRLSCAASGFTFSNYGIHWVRQAPGKGLEWVAAIWYDGSKKYYADSVKGRFTISRDNSKNTLDLQMNSLRAEDTAVYYCARSNYDILTRYYQYGINVWGQGTTVTVSS

SEQ ID NO: 80 - protein, 125 aa, Homo sapiens
QVQLVESGGGVVQPGGSLRLSCAASGFTFSRYGIHWVRQAPGKGLEWVAAIWYDASKTYYADSVKGRFTISRDNSSNTLYLQMNSLRAEDTAVYYCARSNYDILTRYYQYGLDVWGQGTTVTVSS

SEQ ID NO: 81 - protein, 125 aa, Homo sapiens
QVQLVESGGGVVQPGRSLRLSCAASGFTFSNHGIHWVRQAPGKGLEWVAAIWYDGSKKYYADSVKGRFTISRDNSKNTLDLQMNSLRAEDTAVYYCARSNYDILTRYYQYGINVWGQGTTVTVSS

SEQ ID NO: 82 - protein, 125 aa, Homo sapiens
QVQLVESGGGVVQPGESLRLSCAASGFTFSRYGIHWVRQAPGKGLEWVAAIWYDASKDYYADSVKGRFTISRDNSENTLYLQMNSLRAEDTAVYYCARSNYDILTRYYQYGLDVWGQGTTVTVSS

SEQ ID NO: 83 - protein, 125 aa, Homo sapiens
QVQLVESGGGVVQPGRSLRLSCAASGFTFSRHGIHWVRQAPGKGLEWVAAIWYDGSKKYYADSVKGRFTISRDNSKNTLDLQMNSLRAEDTAVYYCARSNYDILTRYYQYGINVWGQGTTVTVSS

SEQ ID NO: 84 - protein, 125 aa, Homo sapiens
QVQLVESGGGVVQPGGSLRLSCAASGFTFSRYGIHWVRQAPGKGLEWVAAIWYDASKKYYADSVKGRFTISRDNSSNTLYLQMNSLRAEDTAVYYCARSNYDILTRYYQYGLDVWGQGTTVTVSS

SEQ ID NO: 85 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNFDILTRYYQYGINVWGQGTTVTVSS

SEQ ID NO: 86 - protein, 125 aa, Homo sapiens
QVQLVESGGGVVQPGESLRLSCAASGFTFSRYGIHWVRQAPGKGLEWVAAIWYDASKKYYADSVKGRFTISRDNSENTLYLQMNSLRAEDTAVYYCARSNYDILTRYYQYGLDVWGQGTTVTVSS

SEQ ID NO: 87 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNFDVLTRYYQYGINVWGQGTTVTVSS

SEQ ID NO: 88 - protein, 121 aa, Homo sapiens
EVQLVQSGAEVKKPGESLKISCKGSGYSFTSYWIGWVRQMPGKCLEWMGIIYPGDSDTRYSPSFQGQVTISADKSISTAYLQWSSLKASDTAMYYCARGGTTIFGVVFEYWGQGTLVTVSS

SEQ ID NO: 89 - protein, 107 aa, Homo sapiens
DIQMTQSPSSLSASVGDRVTITCRASQSINNYLNWYQQKPGKAPKLLIYGASSLQSGVPSRFSGSGSGTDFTLTISSLQPEDFATYYCQQTYSTPWTFGCGTKVEIK

SEQ ID NO: 90 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDVLTRYYQYGINVWGQGTTVTVSS

SEQ ID NO: 91 - protein, 125 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYQYGLDVWGQGTTVTVSS

SEQ ID NO: 92 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTRYYQYGINVWGQGTTVTVSS

SEQ ID NO: 93 - protein, 125 aa, Homo sapiens
QVQLVESGGGVVQPGRSLRLSCAASGFTFSRYGIHWVRQAPGKGLEWVAAIWYDASHKYYADSVKGRFTISRDNSKNTLYLQMNSLRAEDTAVYYCARSNYDILTRYYQYGINVWGQGTTVTVSS

SEQ ID NO: 94 - protein, 125 aa, Homo sapiens
QVQLVESGGGVVQPGRSLRLSCAASGFTFSRYGIHWVRQAPGKGLEWVAAIWYDASNKYYADSVKGRFTISRDNSKNTLYLQMNSLRAEDTAVYYCARSNYDILTRYYQYGINVWGQGTTVTVSS

SEQ ID NO: 95 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTGHYQYGINVWGQGTTVTVSS

SEQ ID NO: 96 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTGYYQYGINVWGQGTTVTVSS

SEQ ID NO: 97 - protein, 125 aa, Homo sapiens
QVQLVESGGGVVQPGRSLRLSCAASGFTFSNHGIHWVRQAPGKGLEWVAAIWYDASKKYYADSVKGRFTISRDNSKNTLYLQMNSLRAEDTAVYYCARSNYDILTRYYQYGINVWGQGTTVTVSS

SEQ ID NO: 98 - protein, 125 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDVLTRYYQYGINVWGQGTTVTVSS

SEQ ID NO: 99 - protein, 107 aa, Homo sapiens
AIQLTQSPSSLSASVGDRVTITCRASQGISSALAWYQQKPGKAPKLLIYDASSLENGVPSRFSGSGSGTDFTLTISSLQPEDFATYYCQQFNSYPRTFGQGTKVEIK

SEQ ID NO: 100 - protein, 107 aa, Homo sapiens
AIQLTQSPSSLSASVGDRVTITCRASQGISSALAWYQQKPGKAPKLLIYDASGLENGVPSRFSGSGSGTDFTLTISSLQPEDFATYYCQQFKSYPRTFGQGTKVEIK

SEQ ID NO: 101 - protein, 107 aa, Homo sapiens
AIQLTQSPSSLSASVGDRVTITCRASQGISSALAWYQQKPGKAPKLLIYDASGLESGVPSRFSGSGSGTDFTLTISSLQPEDFATYYCQQFNSYPRTFGQGTKVEIK

SEQ ID NO: 102 - protein, 10 aa, Homo sapiens
  VARIANT 6: note = X = R or N
GFTFSXYGIH

SEQ ID NO: 103 - protein, 17 aa, Homo sapiens
  VARIANT 6: note = X = G or A
  VARIANT 9: note = X = K or T or D
AIWYDXSKXYYADSVKG

SEQ ID NO: 104 - protein, 16 aa, Homo sapiens
  VARIANT 8: note = X = R or G
  VARIANT 9: note = X = Y or H
  VARIANT 11: note = X = Q or S
  VARIANT 12: note = X = Y or H
  VARIANT 14: note = X = I or L
  VARIANT 15: note = X = N or D
SNYDILTXXYXXGXXV

SEQ ID NO: 105 - protein, 7 aa, Homo sapiens
  VARIANT 7: note = X = R or G or E
DASSLEX

SEQ ID NO: 106 - protein, 9 aa, Homo sapiens
  VARIANT 4: note = X = R or N
QQFXSYPRT

SEQ ID NO: 107 - protein, 17 aa, Homo sapiens
AIWYDGSKKYYADSVKG

SEQ ID NO: 108 - protein, 16 aa, Homo sapiens
SNYDILTRYYQYGINV

SEQ ID NO: 109 - protein, 5 aa, Homo sapiens
NYGIH

SEQ ID NO: 110 - protein, 17 aa, Homo sapiens
AIWYDASKKYYADSVKG

SEQ ID NO: 111 - protein, 16 aa, Homo sapiens
SNYDILTRYYSHGINV

SEQ ID NO: 112 - protein, 17 aa, Homo sapiens
AIWYDASKDYYADSVKG

SEQ ID NO: 113 - protein, 16 aa, Homo sapiens
SNYDILTGHYQYGINV

SEQ ID NO: 114 - protein, 7 aa, Homo sapiens
DASSLEN

SEQ ID NO: 115 - protein, 9 aa, Homo sapiens
QQFNSYPRT

SEQ ID NO: 116 - protein, 125 aa, Homo sapiens
  VARIANT 16: note = X = R or G or E
  VARIANT 31: note = X = R or N
  VARIANT 44: note = X = G or C
  VARIANT 55: note = X = G or A
  VARIANT 58: note = X = K or T or D
  VARIANT 76: note = X = K or E or S
  VARIANT 80: note = X = Y or D
  VARIANT 106: note = X = R or G
  VARIANT 107: note = X = Y or H
  VARIANT 109: note = X = Q or S
  VARIANT 110: note = X = Y or H
  VARIANT 112: note = X = I or L
  VARIANT 113: note = X = N or D
QVQLVESGGGVVQPGXSLRLSCAASGFTFSXYGIHWVRQAPGKXLEWVAAIWYDXSKXYYADSVKGRFTISRDNSXNTLXLQMNSLRAEDTAVYYCARSNYDILTXXYXXGXXVWGQGTTVTVSS

SEQ ID NO: 117 - protein, 107 aa, Homo sapiens
  VARIANT 55: note = X = N or S
  VARIANT 92: note = X = K or N
  VARIANT 100: note = X = Q or C
AIQLTQSPSSLSASVGDRVTITCRASQGISSALAWYQQKPGKAPKLLIYDASSLEXGVPSRFSGSGSGTDFTLTISSLQPEDFATYYCQQFXSYPRTFGXGTKVEIK

SEQ ID NO: 118 - protein, 5 aa, Homo sapiens
SYWIG

SEQ ID NO: 119 - protein, 17 aa, Homo sapiens
IIYPGDSDTRYSPSFQG

SEQ ID NO: 120 - protein, 12 aa, Homo sapiens
GGTTIFGVVFEY

SEQ ID NO: 121 - protein, 11 aa, Homo sapiens
RASQSINNYLN

SEQ ID NO: 122 - protein, 7 aa, Homo sapiens
GASSLQS

SEQ ID NO: 123 - protein, 9 aa, Homo sapiens
QQTYSTPWT

SEQ ID NO: 124 - protein, 13 aa, Homo sapiens
QSIMGVIYVAFDV

SEQ ID NO: 125 - protein, 11 aa, Homo sapiens
RASQSISSYLN

SEQ ID NO: 126 - protein, 7 aa, Homo sapiens
AASSLQS

SEQ ID NO: 127 - protein, 9 aa, Homo sapiens
QQSYSNPWT

SEQ ID NO: 128 - protein, 12 aa, Homo sapiens
GGATIFGVVFDY

SEQ ID NO: 129 - protein, 11 aa, Homo sapiens
RASQSINSYLS

SEQ ID NO: 130 - protein, 9 aa, Homo sapiens
QQSYSTPPT

SEQ ID NO: 131 - protein, 5 aa, Homo sapiens
SYYWS

SEQ ID NO: 132 - protein, 16 aa, Homo sapiens
YIYYSGSTKYNPSLKS

SEQ ID NO: 133 - protein, 14 aa, Homo sapiens
GLAAAGDYYYGMDV

SEQ ID NO: 134 - protein, 11 aa, Homo sapiens
RASQGISSALA

SEQ ID NO: 135 - protein, 9 aa, Homo sapiens
QQFNSYPLT

SEQ ID NO: 136 - protein, 13 aa, Homo sapiens
QSIVATITWAFDI

SEQ ID NO: 137 - protein, 11 aa, Homo sapiens
RASQGISSYLA

SEQ ID NO: 138 - protein, 7 aa, Homo sapiens
AASTLQS

SEQ ID NO: 139 - protein, 9 aa, Homo sapiens
QQLNSYPYT

SEQ ID NO: 140 - protein, 7 aa, Homo sapiens
SGSYNWG

SEQ ID NO: 141 - protein, 16 aa, Homo sapiens
SIYYSGSTYYNPSLKS

SEQ ID NO: 142 - protein, 7 aa, Homo sapiens
HTGGFDY

SEQ ID NO: 143 - protein, 11 aa, Homo sapiens
RASQSVSSNLA

SEQ ID NO: 144 - protein, 7 aa, Homo sapiens
GASIRAT

SEQ ID NO: 145 - protein, 9 aa, Homo sapiens
QQYNNWPLT

SEQ ID NO: 146 - protein, 5 aa, Homo sapiens
SYWIA

SEQ ID NO: 147 - protein, 17 aa, Homo sapiens
HGGYNYGFTDYYYGMDV

SEQ ID NO: 148 - protein, 16 aa, Homo sapiens
RSSQRLVHSDGDTYLS

SEQ ID NO: 149 - protein, 7 aa, Homo sapiens
KISNRFS

SEQ ID NO: 150 - protein, 9 aa, Homo sapiens
MQATQFPYT

SEQ ID NO: 151 - protein, 13 aa, Homo sapiens
RSIAGTTSWSFDL

SEQ ID NO: 152 - protein, 11 aa, Homo sapiens
RTSQDIRNDLG

SEQ ID NO: 153 - protein, 9 aa, Homo sapiens
LQDYNYPLT

SEQ ID NO: 154 - protein, 16 aa, Homo sapiens
YIYYSGRTNYNPSLKS

SEQ ID NO: 155 - protein, 9 aa, Homo sapiens
HHWGSALDY

SEQ ID NO: 156 - protein, 16 aa, Homo sapiens
RSSQSLVYSDGNTYLN

SEQ ID NO: 157 - protein, 7 aa, Homo sapiens
KVSNRDS

SEQ ID NO: 158 - protein, 9 aa, Homo sapiens
MQGTHWPWT

SEQ ID NO: 159 - protein, 125 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTGHYQYGINVWGQGTTVTVSS

SEQ ID NO: 160 - protein, 5 aa, Homo sapiens
GGGGS

SEQ ID NO: 161 - protein, 10 aa, Homo sapiens
GGGGSGGGGS

SEQ ID NO: 162 - protein, 15 aa, Homo sapiens
GGGGSGGGGSGGGGS

SEQ ID NO: 163 - protein, 226 aa, Homo sapiens
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 164 - protein, 226 aa, Homo sapiens
DKTHTCPPCPAPEAEGAPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 165 - protein, 226 aa, Homo sapiens
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 166 - protein, 226 aa, Homo sapiens
DKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 167 - protein, 227 aa, Homo sapiens
ERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPCREEMTKNQVSLWCLVKGFYPSDIAVEWESNGQPENNYKTTPPMLDSDGSFFLYSKLTVDKSRWQQGNVFSCSVMHEALHNHYTQKSLSLSPG

SEQ ID NO: 168 - protein, 227 aa, Homo sapiens
ERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPG

SEQ ID NO: 169 - protein, 103 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 170 - protein, 98 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 171 - protein, 107 aa, Homo sapiens [sequence omitted]

SEQ ID NO: 172 - protein, 470 aa, Homo sapiens
QVQLQQWGAGLLKPSETLSLTCAVYGGSFSDYYWSWVRQPPGKCLEWIGEITHSGSTNYNPSLKSRVTISVDTSKNQFSLKLSSVTAADTAVYYCARGGLDIFTGPYYFDSWGQGTLVTVSSGGGGSGGGGSGGGGSDIQMTQSPSSLSASVGDRVTITCRASQEIRKDLAWYQQKPGKAPKLLIYGASSLRSGVPSRFSGSGSGTDFTLTISSLQPEDFATYYCQQDYNYPPTFGCGTKVEIKDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 173 - protein, 473 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYQYGLDVWGQGTTVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFKSYPRTFGCGTKVEIKDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 174 - protein, 470 aa, Homo sapiens; identical to SEQ ID NO: 172

SEQ ID NO: 175 - protein, 454 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTRYYQYGLDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 176 - protein, 214 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFKSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 177 - protein, 470 aa, Homo sapiens; identical to SEQ ID NO: 172

SEQ ID NO: 178 - protein, 473 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYSHGINVWGQGTTVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPRTFGCGTKVEIKDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 179 - protein, 470 aa, Homo sapiens; identical to SEQ ID NO: 172

SEQ ID NO: 180 - protein, 454 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTRYYSHGINVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 181 - protein, 214 aa, Homo sapiens
AIQLTQSPSSLSASVGDRVTITCRASQGISSALAWYQQKPGKAPKLLIYDASSLESGVPSRFSGSGSGTDFTLTISSLQPEDFATYYCQQFNSYPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 182 - protein, 469 aa, Homo sapiens
EVQLVQSGAEVKKPGESLKISCKGSGYSFTSYWIGWVRQMPGKCLEWMGIIYPGDSDTRYSPSFQGQVTISADKSISTAYLQWSSLKASDTAMYYCARGGTTIFGVVFEYWGQGTLVTVSSGGGGSGGGGSGGGGSDIQMTQSPSSLSASVGDRVTITCRASQSINNYLNWYQQKPGKAPKLLIYGASSLQSGVPSRFSGSGSGTDFTLTISSLQPEDFATYYCQQTYSTPWTFGCGTKVEIKDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 183 - protein, 473 aa, Homo sapiens; identical to SEQ ID NO: 173

SEQ ID NO: 184 - protein, 469 aa, Homo sapiens; identical to SEQ ID NO: 182

SEQ ID NO: 185 - protein, 454 aa, Homo sapiens; identical to SEQ ID NO: 175

SEQ ID NO: 186 - protein, 214 aa, Homo sapiens; identical to SEQ ID NO: 176

SEQ ID NO: 187 - protein, 469 aa, Homo sapiens; identical to SEQ ID NO: 182

SEQ ID NO: 188 - protein, 473 aa, Homo sapiens; identical to SEQ ID NO: 178

SEQ ID NO: 189 - protein, 469 aa, Homo sapiens; identical to SEQ ID NO: 182

SEQ ID NO: 190 - protein, 454 aa, Homo sapiens; identical to SEQ ID NO: 180

SEQ ID NO: 191 - protein, 214 aa, Homo sapiens; identical to SEQ ID NO: 181

SEQ ID NO: 192 - protein, 125 aa, Homo sapiens
  VARIANT 16: note = X = R or G or E
  VARIANT 31: note = X = R or N
  VARIANT 44: note = X = G or C
  VARIANT 58: note = X = K or T or D
  VARIANT 76: note = X = K or E or S
  VARIANT 106: note = X = R or G
  VARIANT 107: note = Y or H
  VARIANT 109: note = X = Q or S
  VARIANT 110: note = X = Y or H
  VARIANT 112: note = X = I or L
  VARIANT 113: note = X = N or D
QVQLVESGGGVVQPGXSLRLSCAASGFTFSXYGIHWVRQAPGKXLEWVAAIWYDASKXYYADSVKGRFTISRDNSXNTLYLQMNSLRAEDTAVYYCARSNYDILTXXYXXGXXVWGQGTTVTVSS

SEQ ID NO: 193 - protein, 107 aa, Homo sapiens
  VARIANT 92: note = X = K or N
  VARIANT 100: note = X = Q or C
AIQLTQSPSSLSASVGDRVTITCRASQGISSALAWYQQKPGKAPKLLIYDASSLESGVPSRFSGSGSGTDFTLTISSLQPEDFATYYCQQFXSYPRTFGXGTKVEIK

SEQ ID NO: 194 - protein, 17 aa, Homo sapiens
  VARIANT 9: note = X = K, T or D
AIWYDASKXYYADSVKG

SEQ ID NO: 195 - protein, 125 aa, Homo sapiens
QVQLVESGGGVVQPGESLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSS

SEQ ID NO: 196 - protein, 125 aa, Homo sapiens
  VARIANT 16: note = X = R or G or E
  VARIANT 31: note = X = R or N
  VARIANT 32: note = Y or H
  VARIANT 44: note = X = G or C
  VARIANT 55: note = X = G or A
  VARIANT 57: note = X = K or H or N
  VARIANT 58: note = X = K or T or D
  VARIANT 59: note = K or E or S
  VARIANT 76: note = X = Y or D
  VARIANT 103: note = X = Y or F
  VARIANT 107: note = X = I or V
  VARIANT 106: note = X = R or G
  VARIANT 110: note = X = Y or H
  VARIANT 112: note = X = I or L
  VARIANT 113: note = X = N or D
  VARIANT 109: note = X = Q or S
  VARIANT 108: note = Y or H
QVQLVESGGGVVQPGXSLRLSCAASGFTFSXXGIHWVRQAPGKXLEWVAAIWYDXSXXYYADSVKGRFTISRDNSXNTLXLQMNSLRAEDTAVYYCARSNXDXLTXXYXXGXXVWGQGTTVTVSS

SEQ ID NO: 197 - protein, 107 aa, Homo sapiens
  VARIANT 53: note = X = S or G
  VARIANT 56: note = X = N or S
  VARIANT 92: note = X = K or N
  VARIANT 100: note = X = Q or C
AIQLTQSPSSLSASVGDRVTITCRASQGISSALAWYQQKPGKAPKLLIYDASXLEXGVPSRFSGSGSGTDFTLTISSLQPEDFATYYCQQFXSYPRTFGXGTKVEIK

SEQ ID NO: 198 - skipped

SEQ ID NO: 199 - protein, 17 aa, Homo sapiens
  VARIANT 6: note = X = G or A
  VARIANT 8: note = X = K or H or N
  VARIANT 9: note = X = K or T or D
AIWYDXSXXYYADSVKG

SEQ ID NO: 200 - protein, 16 aa, Homo sapiens
  VARIANT 3: note = X = Y or F
  VARIANT 5: note = X = I or V
  VARIANT 8: note = X = R or G
  VARIANT 9: note = X = Y or H
  VARIANT 11: note = X = Q or S
  VARIANT 12: note = X = Y or H
  VARIANT 14: note = X = I or L
  VARIANT 15: note = X = N or D
SNXDXLTXXYXXGXXV

SEQ ID NO: 201 - protein, 7 aa, Homo sapiens
  VARIANT 4: note = X = S or G
  VARIANT 7: note = X = N or S
DASXLEX

SEQ ID NO: 202 - protein, 5 aa, Homo sapiens
NHGIH

SEQ ID NO: 203 - protein, 5 aa, Homo sapiens
RHGIH

SEQ ID NO: 204 - protein, 17 aa, Homo sapiens
AIWYDSSKKYYADSVKG

SEQ ID NO: 205 - protein, 17 aa, Homo sapiens
AIWYDGSHKYYADSVKG

SEQ ID NO: 206 - protein, 17 aa, Homo sapiens
AIWYDGSNKYYADSVKG

SEQ ID NO: 207 - protein, 17 aa, Homo sapiens
AIWYDASHKYYADSVKG

SEQ ID NO: 208 - protein, 17 aa, Homo sapiens
AIWYDASNKYYADSVKG

SEQ ID NO: 209 - protein, 16 aa, Homo sapiens
SNYDILTRHYQYGINV

SEQ ID NO: 210 - protein, 16 aa, Homo sapiens
SNYDILTGYYQYGINV

SEQ ID NO: 211 - protein, 16 aa, Homo sapiens
SNYDILTRYYQHGINV

SEQ ID NO: 212 - protein, 16 aa, Homo sapiens
SNFDILTRYYQYGINV

SEQ ID NO: 213 - protein, 16 aa, Homo sapiens
SNFDVLTRYYQYGINV

SEQ ID NO: 214 - protein, 16 aa, Homo sapiens
SNYDVLTRYYQYGINV

SEQ ID NO: 215 - protein, 7 aa, Homo sapiens
DASGLEN

SEQ ID NO: 216 - protein, 7 aa, Homo sapiens
DASGLES

SEQ ID NO: 217 - skipped

SEQ ID NO: 218 - protein, 18 aa, Homo sapiens
GSTSGSGKPGSGEGSTKG

SEQ ID NO: 219 - protein, 21 aa, Homo sapiens
SRGGGGSGGGGSGGGGSLEMA

SEQ ID NO: 220 - protein, 22 aa, Homo sapiens
GSRGGGGSGGGGSGGGGSLEMA

SEQ ID NO: 221 - protein, 98 aa, Homo sapiens
ASTKGPSVFPLAPSSKSTSGGTAWLGCEVTDYFPEPVTVSWNSGALTSGVHTFPAVLESSGLYSLSSVVTVPSSSLGTQTYICNVNHKPSNTKVDKKV

SEQ ID NO: 222 - protein, 6 aa, Homo sapiens
HHHHHH

SEQ ID NO: 223 - protein, 98 aa, Homo sapiens
ASTKGPSVFPLAPSSKSTSGGTAALGCLVKDYFPEPVTVSWNSGALTSGVHTFPAVLKSSGLYSLSSVVTVPSSSLGTQTYICNVNHKPSNTKVDKKV

SEQ ID NO: 224 - protein, 105 aa, Homo sapiens
QPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 225 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 226 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 227 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 228 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 229 - protein, 231 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 230 - protein, 231 aa, Homo sapiens
EPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 231 - protein, 231 aa, Homo sapiens
EPKSCDKTHTCPPCPAPEAAGGPSVFLFPPKPKDTLMISRTPEVTCVVVSVSHEDPEVKFNWYVDGVEVHNAKTKPREEQYNSTYRVVSVLTVLHQDWLNGKEYKCKVSNKALPAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 232 - protein, 231 aa, Homo sapiens
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYVYPPSREEMTKNQVSLTCLVKGFYPSDIAVEWESNGQPENNYKTTPPVLDSDGSFALVSKLTVDKSRWQQGNVFSCSVMHEALHNHYTQKSLSLSPG

SEQ ID NO: 233 - protein, 231 aa, Homo sapiens
EPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYVLPPSREEMTKNQVSLLCLVKGFYPSDIAVEWESNGQPENNYLTWPPVLDSDGSFFLYSKLTVDKSRWQQGNVFSCSVMHEALHNHYTQKSLSLSPG

SEQ ID NO: 234 - protein, 471 aa, Homo sapiens
QVQLQQWGAGLLKPSETLSLTCAVYGGSFSDYYWSWVRQPPGKCLEWIGEITHSGSTNYNPSLKSRVTISVDTSKNQFSLKLSSVTAADTAVYYCARGGLDIFTGPYYFDSWGQGTLVTVSSGGGGSGGGGSGGGGSDIQMTQSPSSLSASVGDRVTITCRASQEIRKDLAWYQQKPGKAPKLLIYGASSLRSGVPSRFSGSGSGTDFTLTISSLQPEDFATYYCQQDYNYPPTFGCGTKVEIKERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVCTLPPSREEMTKNQVSLSCAVKGFYPSDIAVEWESNGQPENNYKTTPPMLDSDGSFFLVSKLTVDKSRWQQGNVFSCSVMHEALHNHYTQKSLSLSPG

SEQ ID NO: 235 - protein, 450 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTRYYQYGLDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPCREEMTKNQVSLWCLVKGFYPSDIAVEWESNGQPENNYKTTPPMLDSDGSFFLYSKLTVDKSRWQQGNVFSCSVMHEALHNHYTQKSLSLSPG

SEQ ID NO: 236 - protein, 214 aa, Homo sapiens; identical to SEQ ID NO: 176

SEQ ID NO: 237 - protein, 471 aa, Homo sapiens; identical to SEQ ID NO: 234

SEQ ID NO: 238 - protein, 450 aa, Homo sapiens
QVQLVESGGGVVQPGGSLRLSCAASGFTFSRYGIHWVRQAPGKGLEWVAAIWYDASKTYYADSVKGRFTISRDNSKNTLYLQMNSLRAEDTAVYYCARSNYDILTRYYSHGINVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKSCVECPPCPAPPVAGKSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPCREEMTKNQVSLWCLVKGFYPSDIAVEWESNGQPENNYKTTPPMLDSDGSFFLYSKLTVDKSRWQQGNVFSCSVMHEALHNHYTQKSLSLSPG

SEQ ID NO: 239 - protein, 214 aa, Homo sp.; identical to SEQ ID NO: 181

SEQ ID NO: 240 - protein, 14 aa, Homo sapiens
ZDGNEEMGGITQTP

SEQ ID NO: 241 - protein, 642 aa, Homo sapiens
EVQLVESGGGLVQPGNSLRLSCAASGFTFSSFGMSWVRQAPGKGLEWVSSISGSGSDTLYADSVKGRFTISRDNAKTTLYLQMNSLRPEDTAVYYCTIGGSLSRSSQGTLVTVSSGGGGSGGGSQVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIKGGGGSGGGSDIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSSHHHHHHHHHH

SEQ ID NO: 242 - protein, 248 aa, Homo sapiens
QVQLVQSGAEVKKPGASVKVSCKASGYTFTDYIMHWVRQAPGQGLEWMGYINPYNDGSKYTEKFQGRVTMTSDTSISTAYMELSRLRSDDTAVYYCARGTYYYGPQLFDYWGQGTTVTVSSGGGGSGGGGSGGGGSDIVMTQTPLSLSVTPGQPASISCKSSQSLETSTGTTYLNWYLQKPGQSPQLLIYRVSKRFSGVPDRFSGSGSGTDFTLKISRVEAEDVGVYYCLQLLEDPYTFGQGTKLEIK

SEQ ID NO: 243 - protein, 251 aa, Homo sapiens
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSRRTFGGGTKVEIKGGGGSGGGGSGGGGSGGGGSQVQLVQSGAEVKKPGASVKVSCKASGFNIKDYYMHWVRQAPGQRLEWMGWIDLENANTIYDAKFQGRVTITRDTSASTAYMELSSLRSEDTAVYYCARDAYGRYFYDVWGQGTLVTVSS

SEQ ID NO: 244 - protein, 115 aa, Homo sapiens [sequence omitted]

SEQ ID NO: 245 - protein, 244 aa, Homo sapiens
QVQLQQWGAGLLKPSETLSLTCAVYGGSFSDYYWSWVRQPPGKCLEWIGEITHSGSTNYNPSLKSRVTISVDTSKNQFSLKLSSVTAADTAVYYCARGGLDIFTGPYYFDSWGQGTLVTVSSGGGGSGGGGSGGGGSDIQMTQSPSSLSASVGDRVTITCRASQEIRKDLAWYQQKPGKAPKLLIYGASSLRSGVPSRFSGSGSGTDFTLTISSLQPEDFATYYCQQDYNYPPTFGCGTKVEIK

SEQ ID NO: 246 - protein, 247 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYQYGLDVWGQGTTVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFKSYPRTFGCGTKVEIK

SEQ ID NO: 247 - protein, 247 aa, Homo sapiens
QVQLVESGGGVVQPGGSLRLSCAASGFTFSRYGIHWVRQAPGKCLEWVAAIWYDASKTYYADSVKGRFTISRDNSKNTLYLQMNSLRAEDTAVYYCARSNYDILTRYYSHGINVWGQGTTVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFNSYPRTFGCGTKVEIK

SEQ ID NO: 248 - protein, 243 aa, Homo sapiens
EVQLVQSGAEVKKPGESLKISCKGSGYSFTSYWIGWVRQMPGKCLEWMGIIYPGDSDTRYSPSFQGQVTISADKSISTAYLQWSSLKASDTAMYYCARGGTTIFGVVFEYWGQGTLVTVSSGGGGSGGGGSGGGGSDIQMTQSPSSLSASVGDRVTITCRASQSINNYLNWYQQKPGKAPKLLIYGASSLQSGVPSRFSGSGSGTDFTLTISSLQPEDFATYYCQQTYSTPWTFGCGTKVEIK

SEQ ID NO: 249 - protein, 244 aa, Homo sapiens
QVQLQQWGAGLLKPSETLSLTCAVYGGSFSDYYWSWVRQPPGKGLEWIGEITHSGSTNYNPSLKSRVTISVDTSKNQFSLKLSSVTAADTAVYYCARGGLDIFTGPYYFDSWGQGALVTVSSGGGGSGGGGSGGGGSEIVLTQSPSSLSASVGDRVTITCRASQEIRKDLAWYQQKPGKAPKVLIYGASSLRSGVPSSFSGSGSGTDFTLTISSLQPEDFATYYCQQDYNYPPTFGQGTKVEIK

SEQ ID NO: 250 - protein, 247 aa, Homo sapiens
QVQLVESGGGVVQPGRSLRLSCAASGFTFSRYGIHWVRQAPGKGLEWVAAIWYDGSKKYYADSVKGRFTISRDNSKNTLDLQMNSLRAEDTAVYYCARSNYDILTRYYQYGINVWGQGTTVTVSSGGGGSGGGGSGGGGSAIQLTQSPSSLSASVGDRVTITCRASQGISSALAWYQQKPGKAPKLLIYDASSLENGVPSRFSGSGSGTDFTLTISSLQPEDFATYYCQQFKSYPRTFGQGTKVEIK

SEQ ID NO: 251 - protein, 247 aa, Homo sapiens
QVQLVESGGGVVQPGRSLRLSCAASGFTFSRYGIHWVRQAPGKGLEWVAAIWYDASKKYYADSVKGRFTISRDNSKNTLYLQMNSLRAEDTAVYYCARSNYDILTRYYQYGLDVWGQGTTVTVSSGGGGSGGGGSGGGGSAIQLTQSPSSLSASVGDRVTITCRASQGISSALAWYQQKPGKAPKLLIYDASSLESGVPSRFSGSGSGTDFTLTISSLQPEDFATYYCQQFKSYPRTFGQGTKVEIK

SEQ ID NO: 252 - protein, 244 aa, Homo sapiens
QVQLQQWGAGLLKPSETLSLTCAVYGGSFSDYYWSWVRQPPGKCLEWIGEITHSGSTNYNPSLKSRVTISVDTSKNQFSLKLSSVTAADTAVYYCARGGLDIFTGPYYFDSWGQGALVTVSSGGGGSGGGGSGGGGSEIVLTQSPSSLSASVGDRVTITCRASQEIRKDLAWYQQKPGKAPKVLIYGASSLRSGVPSSFSGSGSGTDFTLTISSLQPEDFATYYCQQDYNYPPTFGCGTKVEIK

SEQ ID NO: 253 - protein, 329 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 254 - protein, 451 aa, Homo sapiens
QVQLQQWGAGLLKPSETLSLTCAVYGGSFSDYYWSWVRQPPGKGLEWIGEITHSGSTNYNPSLKSRVTISVDTSKNQFSLKLSSVTAADTAVYYCARGGLDIFTGPYYFDSWGQGALVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 255 - protein, 214 aa, Homo sapiens
EIVLTQSPSSLSASVGDRVTITCRASQEIRKDLAWYQQKPGKAPKVLIYGASSLRSGVPSSFSGSGSGTDFTLTISSLQPEDFATYYCQQDYNYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 256 - protein, 450 aa, Homo sapiens
QVQLVQSGAEVKKPGESLKISCKGSGYSFTSYWIGWVRQMPGKGLELMGIIYPGDSDTRYSPSFQGQVTISADKSISTAYLQWSSLKASDTAMYYCARGGTTIFGVVFEYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 257 - protein, 214 aa, Homo sapiens
EIVLTQSPSSLSTSVGDRVTITCRASQSINNYLNWYQQKPGKAPKLLIYGASSLQSGVPSRFSGSGSGTDFTLTISSLQPEDFATYYCQQTYSTPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 258 - protein, 451 aa, Homo sapiens
EVQLVQSGAEVKKPGESLKISCKGSGYSFTSYWIGWVRQMPGKGLEWMGIIYPGDSDTRYSPSFQGQVTISADKSLSTAYLQWSGLKASDTAMYYCARQSIMGVIYVAFDVWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 259 - protein, 214 aa, Homo sapiens
EIVLTQSPSSLSASVGDRVTITCRASQSISSYLNWYQQKPGKAPKLLIYAASSLQSGVPSRFSGSGSGTDFTLTITSLRPEDFATYYCQQSYSNPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 260 - protein, 450 aa, Homo sapiens
EVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARGGATIFGVVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 261 - protein, 214 aa, Homo sapiens
EIVLTQSPSSLSASVGDRVTITCRASQSINSYLSWFQQKPGKAPKLLIYAASSLQSGVPSRFSGSGSGTDFTLTISSLQPEDFATYYCQQSYSTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 262 - protein, 451 aa, Homo sapiens
QVQLQESGPGLVKPSETLSLTCTVSGGSISSYYWSWIRQPPGKGLEWIGYIYYSGSTKYNPSLKSRVTISVDTSKNQFSLQLSSVTAADTAVYYCATGLAAAGDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 263 - protein, 214 aa, Homo sapiens
EIVLTQSPSSLSASVGDRVTITCRASQGISSALAWYQQKPGKAPKLLIYDASSLESGVPSRFSGGGSGTDFTLTISSLQPEDFASYYCQQFNSYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 264 - protein, 451 aa, Homo sapiens
QVQLVQSGAEVKKPGESLKISCKGSGYSFTSYWIGWVRQMPGKGLEWMGIIYPGDSDTRYSPSFQGQVTISADKSISTAYLQWSSLKASDTAMYYCARQSIVATITWAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 265 - protein, 214 aa, Homo sapiens
EIVLTQSPSFLSASVGDRVTITCRASQGISSYLAWYQQKPGKAPKLLIYAASTLQSGVPSRFSGSGSGTEFTLTVSSLQPEDFATYYCQQLNSYPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 266 - protein, 446 aa, Homo sapiens
QVQLQESGPGLVKPSETLSLTCTVSGGSISSGSYNWGWIRQPPGKGLEWIGSIYYSGSTYYNPSLKSRVTISGDTSKNQFSLKLSSLTAADTAVYYCARHTGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 267 - protein, 214 aa, Homo sapiens
EIVLTQSPATLSVSPGERATLSCRASQSVSSNLAWYQQKPGQAPRLLIYGASIRATGIPARFSGSGSGTEFTLTISSLQSEDFAVYYCQQYNNWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 268 - protein, 455 aa, Homo sapiens
QVQLVQSGAEVKKPGESLRISCKGSGYSFSSYWIAWVRQMPGKGLEWMGIIYPGDSDTRYSPSFQGQVTISADKSISTAYLQWSSLKASDTAMYYCARHGGYNYGFTDYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 269 - protein, 219 aa, Homo sapiens
DIVMTQTPLSSPVTLGQPASISCRSSQRLVHSDGDTYLSWLQQRPGQPPRLLIYKISNRFSGVPDRFSGSGAGTDFTLKISRVEAEDVGVYYCMQATQFPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 270 - protein, 451 aa, Homo sapiens
EVQLVQSGAEVKKPGESLKISCKGSGYSFTSYWIGWVRQMPGKGLEWMGIIYPGDSDTRYSPSFQGQVAISADKSISTAYLQWSSLKASDTAMYYCARRSIAGTTSWSFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 271 - protein, 214 aa, Homo sapiens
EIVLTQSPSSLSASVGDRVTITCRTSQDIRNDLGWYQQKPGNAPKLLIYAASSLQSGVPSRFSGSGSGTDFTLTISSLQPEDFATYYCLQDYNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 272 - protein, 446 aa, Homo sapiens
QVQLQESGPGLVKPSETLSLTCTVSGGSFSSYYWSWIRQPPGKGLEWIGYIYYSGRTNYNPSLKSRVTISIDTSKNQVSLKLSSVTAADTAVYYCASHHWGSALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 273 - protein, 219 aa, Homo sapiens
EIVLTQSPLSLPVTLGQPASISCRSSQSLVYSDGNTYLNWFQQRPGQSPRRLIYKVSNRDSGVPDRFSGSGSGTDFTLKISRVEAEDVGVYYCMQGTHWPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 274 - protein, 451 aa, Homo sapiens
QVQLQQWGAGLLKPSETLSLTCAVYGGSFSDYYWSWVRQPPGKGLEWIGEITHSGSTNYNPSLKSRVTISVDTSKNQFSLKLSSVTAADTAVYYCARGGLDIFTGPYYFDSWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 275 - protein, 214 aa, Homo sapiens
DIQMTQSPSSLSASVGDRVTITCRASQEIRKDLAWYQQKPGKAPKLLIYGASSLRSGVPSRFSGSGSGTDFTLTISSLQPEDFATYYCQQDYNYPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 276 - protein, 17 aa, Homo sapiens
  VARIANT 6: note = X = G or A or S
  VARIANT 8: note = X = K, H orN
  VARIANT 9: note = X = K, T or D
AIWYDXSXXYYADSVKG

SEQ ID NO: 277 - protein, 9 aa, Homo sapiens
  VARIANT 4: note = X = R or N or K
QQFXSYPRT

SEQ ID NO: 278 - protein, 125 aa, Homo sapiens; identical to SEQ ID NO: 196
  VARIANT 16: note = X = R, G, or E
  VARIANT 31: note = X = R or N
  VARIANT 32: note = X = Y or H
  VARIANT 44: note = X = G or C
  VARIANT 55: note = X = G or A or S
  VARIANT 57: note = X = K, H, or N
  VARIANT 58: note = X = K, T, or D
  VARIANT 76: note = X = K, E, or S
  VARIANT 80: note = X = Y or D
  VARIANT 101: note = X = Y or F
  VARIANT 103: note = X = I or V
  VARIANT 106: note = X = R or G
  VARIANT 107: note = X = Y or H
  VARIANT 109: note = X = Q or S
  VARIANT 110: note = X = Y or H
  VARIANT 112: note = X = I or L
  VARIANT 113: note = X = N or D

(19) United States
(12) Patent Application Publication
(10) Pub. No.: US 2026/0356949 A1
(43) Pub. Date: Nov. 12, 2026

(54) CD19 BINDING MOLECULES INCLUDING CD3- AND CD19- BISPECIFIC T CELL ENGAGERS AND RELATED METHODS AND USES

(71) Applicant: Bristol-Myers Squibb Company, Princeton, NJ (US)

(72) Inventors: Ramakrishna CHANDRAN, Hanover, MD (US); Haibin CHEN, San Jose, CA (US); Faisal MALIK, Castro Valley, CA (US); Lore K. FLORIN, Redwood City, CA (US); Kristoff Theodor HOMAN, Newton, MA (US); Kofi Agyare MENSAH, Medford, MA (US); Kajal Digambar PATIL, Medford, MA (US); Steven Anthony SAENZ, Boston, MA (US); Kailyn STEFAN, Watertown, MA (US)

(73) Assignee: Bristol-Myers Squibb Company, Princeton, NJ (US)

(21) Appl. No.: 19/454,265

(22) Filed: Jan. 20, 2026

Related U.S. Application Data
(60) Provisional application No. 63/747,450, filed on Jan. 21, 2025.

(51) Int. Cl. C07K 16/28; A61K 39/00; A61P 37/06

(52) U.S. Cl. CPC C07K 16/2809; A61P 37/06; C07K 16/2803; A61K 2039/505; C07K 2317/31; C07K 2317/524; C07K 2317/526; C07K 2317/53; C07K 2317/55; C07K 2317/565; C07K 2317/622; C07K 2317/92

(57) ABSTRACT

The present invention provides binding molecules comprising a CD19 binding domain and/or a CD3 binding domain. In certain embodiments, the CD19 domain specifically binds to a B cell expressing CD19, and wherein the CD3 domain specifically binds to a T cell expressing CD3. Compositions, methods, and kits comprising the binding molecules are provided for treating diseases and conditions. In certain aspects, the binding molecules, compositions, methods, and kits are useful to deplete B cells.

### CROSS-REFERENCE TO RELATED APPLICATIONS

This application claims priority from U.S. provisional application No. 63/747,450, filed Jan. 21, 2025, entitled “CD19 BINDING MOLECULES INCLUDING CD3- AND CD19-BISPECIFIC T CELL ENGAGERS AND RELATED METHODS AND USES”, the contents of which are incorporated by reference in their entirety.

### INCORPORATION BY REFERENCE OF SEQUENCE LISTING

The present application is being filed along with a Sequence Listing in electronic format. The Sequence Listing is provided as a file entitled 21737-20006.00.xml created Jan. 17, 2026, which is 329,047 bytes in size. The information in the electronic format of the Sequence Listing is incorporated by reference in its entirety.

### FIELD OF DISCLOSURE

This disclosure provides monospecific binding molecules that comprise a binding domain that specifically binds to CD19 and also bispecific binding molecules that comprise a first binding domain that specifically binds to CD19 and a second binding domain that specifically binds to CD3. The disclosure further relates to polynucleotides that encode the binding molecules, including the antibodies or bispecific antibodies. The present disclosure further relates to pharmaceutical compositions, kits, and methods and uses of such binding molecules to reduce and/or deplete B cells in a subject and for the treatment of, for example, autoimmune diseases in which specific targeting, and in some aspects T-cell-mediated killing, of cells that express CD19 is desired.

### BACKGROUND

Autoantibodies and immune complexes drive inflammation and tissue damage (Lou, 2022 et al. J Autoimmun.). B cells are multifunctional lymphocytes that contribute to the pathogenesis via B cell intrinsic, antibody-mediated and T cell-dependent mechanisms. Atisha-Fregoso, et al. J Clin Invest. 2021; and Rubin et al., 2019 Nature Reviews Rheumatology vol 15, 303-315. Blinatumomab, a CD19/CD3 bispecific antibody (bsAb) designed in the BiTE (bispecific T-cell engager) format, is approved by the US Food and Drug Administration for the treatment of relapsed or refractory B-cell precursor acute lymphoblastic leukemia. Zimmerman 2024 International Immunology vol. 27, no. 1; pp. 31-37; Robinson et al., 2018 Blood. 132(5): 521-532. B cells are also drivers of pathogenesis in numerous autoimmune diseases (e.g., systemic lupus erythematosus and Sjogren's disease) with multiple therapeutic agents targeting B cells approved, used as standard or care (SOC), or being developed for autoimmune indications. Kansal et al., 2019 Sci Transl. Med., 11, 1-13.

Systemic lupus erythematosus (SLE) is an autoimmune disease that is considered difficult to diagnose and treat because of the episodic nature of flare-ups, the involvement of multiple organ systems and variable presentation, and severity (Rahman et al., 2008 N. Engl. J. med. 358: 929-939). Autoantibodies to DNA and protein autoantigens are observed in in SLE and contribute to pathogenesis by forming immune complex deposits in various tissues (Shlomchik et al., 2009 Curr. Opin. Immunol. 21, 626-633; Sang et al., 2014 Mol. Immunol. 62, 329-338).

Approaches that inactivate or deplete B cells have been pursued as therapies for different autoimmune diseases such as lupus. These approaches include, but are not limited to, B cell depletion with a monoclonal antibody against the B cell surface marker CD20 and are currently being tested in clinical trials. See Shah et al., 2020 Nature Medicine vol. 26 pp. 1569-1575; Crickx et al., 2020 Kidney Int. 97(5):885-893; Mougiakakos et al., 2021 N Engl. J. Med. 385:6; Mackenson 2022 Nat Med 28, 2124-2132.

However, more effective and safe treatments are needed, including treatments that offer “off-the-shelf” treatment for broad B cell depletion and immune reset, delivering long-term remission and tapering of immuno-suppressive drugs, with lower toxicity risk.

### SUMMARY

An aspect of the inventor provides a binding molecule (i.e. antibody or antigen-binding fragment) comprising an CD19 binding domain comprising a heavy chain variable (VH) region and a light chain variable (VL) region, wherein:
(a) the VH region comprises a heavy chain complementarity determining region 1 (CDR-H1), a heavy chain complementarity determining region 2 (CDR-H2), and a heavy chain complementarity determining region 3 (CDR-H3), wherein the CDR-H1 comprises the amino acid sequence set forth in any one of SEQ ID NOs: 9, 118, 131, 140, or 146, the CDR-H2 comprises the amino acid sequence set forth in any one of SEQ ID NOs: 10, 119, 132, 141, or 154, and the CDR-H3 comprises the amino acid sequence set forth in any one of SEQ ID NOs: 11, 120, 124, 128, 133, 136, 142, 147, 151, or 155; and
the VL region comprises a light chain complementarity determining region 1 (CDR-L1), a light chain complementarity determining region 2 (CDR-L2), and a light chain complementarity determining region 3 (CDR-L3), wherein the CDR-L1 comprises the amino acid sequence set forth in SEQ ID NOs: 14, 121, 125, 129, 134, 137, 143, 148, 152, or 156, the CDR-L2 comprises the amino acid sequence set forth in any one of SEQ ID NOs: 15, 122, 126, 25, 138, 144, 149, or 157, and the CDR-L3 comprises the amino acid sequence set forth in any one of SEQ ID NOs: 16, 123, 127, 130, 135, 139, 145, 150, 153, or 158; or
(b) the VH region comprises a CDR-H1, a CDR-H2, and a CDR-H3 contained within any one of SEQ ID NOs: 12, 32, 34, 44, 46, 48, 50, 52, 54, 56, 58, 60, 62, or 88, and the VL region comprises a CDR-L1, a CDR-L2, and a CDR-L3 contained within any one of SEQ ID NOs: 17, 33, 35, 45, 47, 49, 51, 53, 55, 57, 59, 61, 63, or 89.

In some embodiments, the CDR-H1 comprises the amino acid sequence set forth in any one of SEQ ID NOs: 9, 118, 131, 140, or 146, the CDR-H2 comprises the amino acid sequence set forth in any one of SEQ ID NOs: 10, 119, 132, 141, or 154, and the CDR-H3 comprises the amino acid sequence set forth in any one of SEQ ID NOs: 11, 120, 124, 128, 133, 136, 142, 147, 151, or 155; and
the CDR-L1 comprises the amino acid sequence set forth in SEQ ID NOs: 14, 121, 125, 129, 134, 137, 143, 148, 152, or 156, the CDR-L2 comprises the amino acid sequence set forth in any one of SEQ ID NOs: 15, 122, 126, 25, 138, 144, 149, or 157, and the CDR-L3 comprises the amino acid sequence set forth in any one of SEQ ID NOs: 16, 123, 127, 130, 135, 139, 145, 150, 153, or 158.

In some embodiments, the VH region comprises a CDR-H1, a CDR-H2, and a CDR-H3 contained within SEQ ID NO: 32, and the VL region comprises a CDR-L1, a CDR-L2, and a CDR-L3 contained within SEQ ID NO: 33;
the VH region comprises a CDR-H1, a CDR-H2, and a CDR-H3 contained within SEQ ID NO: 34, and the VL region comprises a CDR-L1, a CDR-L2, and a CDR-L3 contained within SEQ ID NO: 35;
the VH region comprises a CDR-H1, a CDR-H2, and a CDR-H3 contained within SEQ ID NO: 44, and the VL region comprises a CDR-L1, a CDR-L2, and a CDR-L3 contained within SEQ ID NO: 45;
the VH region comprises a CDR-H1, a CDR-H2, and a CDR-H3 contained within SEQ ID NO: 46, and the VL region comprises a CDR-L1, a CDR-L2, and a CDR-L3 contained within SEQ ID NO: 47;
the VH region comprises a CDR-H1, a CDR-H2, and a CDR-H3 contained within SEQ ID NO: 48, and the VL region comprises a CDR-L1, a CDR-L2, and a CDR-L3 contained within SEQ ID NO: 49;
the VH region comprises a CDR-H1, a CDR-H2, and a CDR-H3 contained within SEQ ID NO: 50, and the VL region comprises a CDR-L1, a CDR-L2, and a CDR-L3 contained within SEQ ID NO: 51;
the VH region comprises a CDR-H1, a CDR-H2, and a CDR-H3 contained within SEQ ID NO: 52, and the VL region comprises a CDR-L1, a CDR-L2, and a CDR-L3 contained within SEQ ID NO: 53;
the VH region comprises a CDR-H1, a CDR-H2, and a CDR-H3 contained within SEQ ID NO: 54, and the VL region comprises a CDR-L1, a CDR-L2, and a CDR-L3 contained within SEQ ID NO: 55;
the VH region comprises a CDR-H1, a CDR-H2, and a CDR-H3 contained within SEQ ID NO: 56, and the VL region comprises a CDR-L1, a CDR-L2, and a CDR-L3 contained within SEQ ID NO: 57;
the VH region comprises a CDR-H1, a CDR-H2, and a CDR-H3 contained within SEQ ID NO: 58, and the VL region comprises a CDR-L1, a CDR-L2, and a CDR-L3 contained within SEQ ID NO: 59;
the VH region comprises a CDR-H1, a CDR-H2, and a CDR-H3 contained within SEQ ID NO: 60, and the VL region comprises a CDR-L1, a CDR-L2, a the CDR-L3 contained within SEQ ID NO: 61;
the VH region comprises a CDR-H1, a CDR-H2, and a CDR-H3 contained within SEQ ID NO: 62, and the VL region comprises a CDR-L1, a CDR-L2, and a CDR-L3 contained within SEQ ID NO: 63;
the VH region comprises a CDR-H1, a CDR-H2, and a CDR-H3 contained within SEQ ID NO: 12, and the VL region comprises a CDR-L1, a CDR-L2, and a CDR-L3 contained within SEQ ID NO: 17; or
the VH region comprises a CDR-H1, a CDR-H2, and a CDR-H3 contained within SEQ ID NO: 88, and the VL region comprises a CDR-L1, a CDR-L2, and a CDR-L3 contained within SEQ ID NO: 89.

In some of any embodiments, the VH region comprises the CDR-H1, the CDR-H2, and the CDR-H3 comprising the amino acid sequence set forth in SEQ ID NOS: 9, 10, and 11, respectively, and the VL region comprises the CDR-L1, the CDR-L2, and the CDR-L3 comprising the amino acid sequence set forth in SEQ ID NOS: 14, 15, and 16, respectively; the VH region comprises the CDR-H1, the CDR-H2, and the CDR-H3 comprising the amino acid sequence set forth in SEQ ID NOS: 118, 119, and 120, respectively, and the VL region comprises the CDR-L1, the CDR-L2, and the CDR-L3 comprising the amino acid sequence set forth in SEQ ID NOS: 121, 122, and 123, respectively; the VH region comprises the CDR-H1, the CDR-H2, and the CDR-H3 comprising the amino acid sequence set forth in SEQ ID NOS:118, 119, and 124, respectively, and the VL region comprises the CDR-L1, the CDR-L2, and the CDR-L3 comprising the amino acid sequence set forth in SEQ ID NOS:125, 126, and 127, respectively; the VH region comprises the CDR-H1, the CDR-H2, and the CDR-H3 comprising the amino acid sequence set forth in SEQ ID NOS:118, 119, and 128, respectively, and the VL region comprises the CDR-L1, the CDR-L2, and the CDR-L3 comprising the amino acid sequence set forth in SEQ ID NOS: 129, 126, and 130, respectively; the VH region comprises the CDR-H1, the CDR-H2, and the CDR-H3 comprising the amino acid sequence set forth in SEQ ID NOS: 131, 132, and 133, respectively, and the VL region comprises the CDR-L1, the CDR-L2, and the CDR-L3 comprising the amino acid sequence set forth in SEQ ID NOS: 134, 25, and 135, respectively; the VH region comprises the CDR-H1, the CDR-H2, and the CDR-H3 comprising the amino acid sequence set forth in SEQ ID NOS: 118, 119, and 136, respectively, and the VL region comprises the CDR-L1, the CDR-L2, and the CDR-L3 comprising the amino acid sequence set forth in SEQ ID NOS:137, 138, and 139, respectively; the VH region comprises the CDR-H1, the CDR-H2, and the CDR-H3 comprising the amino acid sequence set forth in SEQ ID NOS:140, 141, and 142, respectively, and the VL region comprises the CDR-L1, the CDR-L2, and the CDR-L3 comprising the amino acid sequence set forth in SEQ ID NOS: 143, 144, and 145, respectively; the VH region comprises the CDR-H1, the CDR-H2, and the CDR-H3 comprising the amino acid sequence set forth in SEQ ID NOS: 146, 119, and 147, respectively, and the VL region comprises the CDR-L1, the CDR-L2, and the CDR-L3 comprising the amino acid sequence set forth in SEQ ID NOS: 148, 149, and 150, respectively; the VH region comprises the CDR-H1, the CDR-H2, and the CDR-H3 comprising the amino acid sequence set forth in SEQ ID NOS: 118, 119, and 151, respectively, and the VL region comprises the CDR-L1, the CDR-L2, and the CDR-L3 comprising the amino acid sequence set forth in SEQ ID NOS: 152, 126, and 153, respectively; or the VH region comprises the CDR-H1, the CDR-H2, and the CDR-H3 comprising the amino acid sequence set forth in SEQ ID NOS:131, 154, and 155, respectively, and the VL region comprises the CDR-L1, the CDR-L2, and the CDR-L3 comprising the amino acid sequence set forth in SEQ ID NOS: 156, 157, and 158, respectively.

In some of any embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 44, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 45; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 46, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 47; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 48, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 49; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 50, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 51; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 52, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 53; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 54, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 55; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 56, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 57; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 58, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 59; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 60, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 61; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 62, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 63; the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 12, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 17; or the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 88, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 89.

In some of any embodiments, the VH region and the VL region comprise the amino acid sequences set forth in SEQ ID NO: 32 and 33, respectively; the VH region and the VL region comprise the amino acid sequences set forth in SEQ ID NO: 34 and 35, respectively; the VH region and the VL region comprise the amino acid sequences set forth in SEQ ID NO: 44 and 45, respectively; the VH region and the VL region comprise the amino acid sequences set forth in SEQ ID NO: 46 and 47, respectively; the VH region and the VL region comprise the amino acid sequences set forth in SEQ ID NO: 48 and 49, respectively; the VH region and the VL region comprise the amino acid sequences set forth in SEQ ID NO: 50 and 51, respectively; the VH region and the VL region comprise the amino acid sequences set forth in SEQ ID NO: 52 and 53, respectively; the VH region and the VL region comprise the amino acid sequences set forth in SEQ ID NO: 54 and 55, respectively; the VH region and the VL region comprise the amino acid sequences set forth in SEQ ID NO: 56 and 57, respectively; the VH region and the VL region comprise the amino acid sequences set forth in SEQ ID NO: 58 and 59, respectively; the VH region and the VL region comprise the amino acid sequences set forth in SEQ ID NO: 60 and 61, respectively; the VH region and the VL region comprise the amino acid sequences set forth in SEQ ID NO: 62 and 63, respectively; the VH region and the VL region comprise the amino acid sequences set forth in SEQ ID NO: 12 and 17, respectively; or the VH region and the VL region comprise the amino acid sequences set forth in SEQ ID NO: 88 and 89, respectively.

In some of any embodiments, the VH region comprises the CDR-H1, a CDR-H2, and a CDR-H3 comprising the amino acid sequence set forth in SEQ ID NOs: 9, 10, and 11, respectively, and the VL region comprises a CDR-L1, a CDR-L2, and a CDR-L3 comprising the amino acid sequence set forth in SEQ ID NOS: 14, 15, and 16, respectively.

In some embodiments, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 12, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 17. In some of any embodiments, the VH region and the VL region comprises the amino acid sequence set forth in SEQ ID NOs: 12 and 17, respectively.

In some of any embodiments, the VH region comprises the CDR-H1, a CDR-H2, and a CDR-H3 comprising the sequences set forth in SEQ ID NOs: 118, 119, and 120, respectively, and the VL region comprises a CDR-L1, a CDR-L2, and a CDR-L3 comprising the sequence set forth in SEQ ID NOS: 121, 122, 123, respectively. In some embodiments, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 88, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 89. In some of any embodiments, the VH region and the VL region comprises the sequence or are the sequence set forth in SEQ ID NOs: 88 and 89, respectively.

In some of any embodiments, the binding molecule is a full-length antibody. In some of any embodiments, the antibody is a human IgG. In some embodiments, the antibody is IgG1. In some of any embodiments, the antibody comprises a kappa light chain constant region.

In some of any embodiments, the antibody comprises:
(a) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 254; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 255;
(b) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 256; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 257;
(c) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 258; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 259;
(d) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 260; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 261;
(e) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 262; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 263;
(f) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 264; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 265;
(g) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 266; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 267;
(h) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 268; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 269;
(i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 270; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 271;
(j) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 272; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 273;
(k) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 274; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 275.

In some of any embodiments, the antibody comprises:
(a) (i) a heavy chain comprising the amino acid sequence of SEQ ID NO: 254; and (ii) a light chain comprising the amino acid sequence of SEQ ID NO: 255;
(b) a heavy chain comprising the amino acid sequence of SEQ ID NO: 256; and (ii) a light chain comprising the amino acid sequence of SEQ ID NO: 257;
(c) a heavy chain comprising the amino acid sequence of SEQ ID NO: 258; and (ii) a light chain comprising the amino acid sequence of SEQ ID NO: 259;
(d) a heavy chain comprising the amino acid sequence of SEQ ID NO: 260; and (ii) a light chain comprising the amino acid sequence of SEQ ID NO: 261;
(e) a heavy chain comprising the amino acid sequence of SEQ ID NO: 262; and (ii) a light chain comprising the amino acid sequence of SEQ ID NO: 263;
(f) a heavy chain comprising the amino acid sequence of SEQ ID NO: 264; and (ii) a light chain comprising the amino acid sequence of SEQ ID NO: 265;
(g) a heavy chain comprising the amino acid sequence of SEQ ID NO: 266; and (ii) a light chain comprising the amino acid sequence of SEQ ID NO: 267;
(h) a heavy chain comprising the amino acid sequence of SEQ ID NO: 268; and (ii) a light chain comprising the amino acid sequence of SEQ ID NO: 269;
(i) a heavy chain comprising the amino acid sequence of SEQ ID NO: 270; and (ii) a light chain comprising the amino acid sequence of SEQ ID NO: 271;
(j) a heavy chain comprising the amino acid sequence of SEQ ID NO: 272; and (ii) a light chain comprising the amino acid sequence of SEQ ID NO: 273; or
(k) a heavy chain comprising the amino acid sequence of SEQ ID NO: 274; and (ii) a light chain comprising the amino acid sequence of SEQ ID NO: 275.

In some of any embodiments, the binding molecule is an antigen-binding fragment. In some embodiments, the antigen-binding fragment is selected from the group consisting of Fab, Fab′, F(ab′)2, Fv, and scFv. In some of any embodiments, the antigen-binding fragment is an scFv. In some embodiments, the scFv comprises a VH-linker-VL arrangement. In some embodiments, the scFv comprises a VL-linker-VH arrangement. In some of any embodiments, the linker is a peptide linker comprising (Gly4Ser)3 (SEQ ID NO: 162) In some of any embodiments, the linker is a peptide linker comprising Gly4Ser (SEQ ID NO: 160). In some of any embodiments, the linker is a peptide linker comprising (Gly4Ser)2 (SEQ ID NO: 161).

In some of any embodiments, the scFv comprises:
(a) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 245;
(b) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 248;
(c) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 249; or
(d) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 252.

In some of any embodiments, the scFv comprises: (a) the amino acid sequence of SEQ ID NO: 245; (b) the amino acid sequence of SEQ ID NO: 248; (c) the amino acid sequence of SEQ ID NO: 249; or (d) the amino acid sequence of SEQ ID NO: 252. In some of any embodiments, the scFv comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 245. In some of any embodiments, the scFv comprises the amino acid sequence of SEQ ID NO: 245. In some of any embodiments, the scFv comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 248. In some of any embodiments, the scFv comprises the amino acid sequence of SEQ ID NO: 248.

Provided herein is a bispecific antibody comprising (a) a first binding domain that binds CD19 and (b) a second binding domain that binds CD3, wherein: the first binding domain that binds CD19 comprises a heavy chain variable (VH) region and a light chain variable (VL) region, wherein: (a) the VH region comprises a heavy chain complementarity determining region 1 (CDR-H1) comprising the amino acid sequence set forth in any one of SEQ ID NOs: 9, 118, 131, 140, or 146, a heavy chain complementarity determining region 2 (CDR-H2) comprising the amino acid sequence set forth in any one of SEQ ID NOs: 10, 119, 132, 141, or 154, and a heavy chain complementarity determining region 3 (CDR-H3) comprising the amino acid sequence set forth in any one of SEQ ID NOs: 11, 120, 124, 128, 133, 136, 142, 147, 151, or 155; and the VL region comprises a light chain complementarity determining region 1 (CDR-L1) comprising the amino acid sequence set forth in SEQ ID NOs: 14, 121, 125, 129, 134, 137, 143, 148, 152, or 156; a light chain complementarity determining region 2 (CDR-L2) comprising the amino acid sequence set forth in any one of SEQ ID NOs: 15, 122, 126, 25, 138, 144, 149, or 157, and a light chain complementarity determining region 3 (CDR-L3) comprising the amino acid sequence set forth in any one of SEQ ID NOs: 16, 123, 127, 130, 135, 139, 145, 150, 153, or 158; or
(b) the VH region comprises a CDR-H1, a CDR-H2, and a CDR-H3 contained within any one of SEQ ID NOs: 12, 32, 34, 44, 46, 48, 50, 52, 54, 56, 58, 60, 62, or 88, and the VL region comprises a CDR-L1, a CDR-L2), and a CDR-L3 contained within any one of SEQ ID NOs: 17, 33, 35, 45, 47, 49, 51, 53, 55, 57, 59, 61, 63, or 89.

In some embodiments, in the first binding domain:
the CDR-H1 comprises the amino acid sequence set forth in any one of SEQ ID NOs: 9, 118, 131, 140, or 146, the CDR-H2 comprises the amino acid sequence set forth in any one of SEQ ID NOs: 10, 119, 132, 141, or 154, and the CDR-H3 comprises the amino acid sequence set forth in any one of SEQ ID NOs: 11, 120, 124, 128, 133, 136, 142, 147, 151, or 155; and
the CDR-L1 comprises the amino acid sequence set forth in SEQ ID NOs: 14, 121, 125, 129, 134, 137, 143, 148, 152, or 156, the CDR-L2 comprises the amino acid sequence set forth in any one of SEQ ID NOs: 15, 122, 126, 25, 138, 144, 149, or 157, and the CDR-L3 comprises the amino acid sequence set forth in any one of SEQ ID NOs: 16, 123, 127, 130, 135, 139, 145, 150, 153, or 158.

In some of any embodiments, in the first binding domain:
the VH region comprises the CDR-H1, the CDR-H2, and the CDR-H3 contained within SEQ ID NO: 32, and the VL region comprises the CDR-L1, the CDR-L2, and the CDR-L3 contained within SEQ ID NO: 33;
the VH region comprises the CDR-H1, the CDR-H2, and the CDR-H3 contained within SEQ ID NO: 34, and the VL region comprises the CDR-L1, the CDR-L2, and the CDR-L3 contained within SEQ ID NO: 35;
the VH region comprises the CDR-H1, the CDR-H2, and the CDR-H3 contained within SEQ ID NO: 44, and the VL region comprises the CDR-L1, the CDR-L2, and the CDR-L3 contained within SEQ ID NO: 45;
the VH region comprises the CDR-H1, the CDR-H2, and the CDR-H3 contained within SEQ ID NO: 46, and the VL region comprises the CDR-L1, the CDR-L2, and the CDR-L3 contained within SEQ ID NO: 47;
the VH region comprises the CDR-H1, the CDR-H2, and the CDR-H3 contained within SEQ ID NO: 48, and the VL region comprises the CDR-L1, the CDR-L2, and the CDR-L3 contained within SEQ ID NO: 49;
the VH region comprises the CDR-H1, the CDR-H2, and the CDR-H3 contained within SEQ ID NO: 50, and the VL region comprises the CDR-L1, the CDR-L2, and the CDR-L3 contained within SEQ ID NO: 51;
the VH region comprises the CDR-H1, the CDR-H2, and the CDR-H3 contained within SEQ ID NO: 52, and the VL region comprises the CDR-L1, the CDR-L2, and the CDR-L3 contained within SEQ ID NO: 53;
the VH region comprises the CDR-H1, the CDR-H2, and the CDR-H3 contained within SEQ ID NO: 54, and the VL region comprises the CDR-L1, the CDR-L2, and the CDR-L3 contained within SEQ ID NO: 55;
the VH region comprises the CDR-H1, the CDR-H2, and the CDR-H3 contained within SEQ ID NO: 56, and the VL region comprises the CDR-L1, the CDR-L2, and the CDR-L3 contained within SEQ ID NO: 57;
the VH region comprises the CDR-H1, the CDR-H2, and the CDR-H3 contained within SEQ ID NO: 58, and the VL region comprises the CDR-L1, the CDR-L2, and the CDR-L3 contained within SEQ ID NO: 59;
the VH region comprises the CDR-H1, the CDR-H2, and the CDR-H3 contained within SEQ ID NO: 60, and the VL region comprises the CDR-L1, the CDR-L2, and the CDR-L3 contained within SEQ ID NO: 61;
the VH region comprises the CDR-H1, the CDR-H2, and the CDR-H3 contained within SEQ ID NO: 62, and the VL region comprises the CDR-L1, the CDR-L2, and the CDR-L3 contained within SEQ ID NO: 63;
the VH region comprises the CDR-H1, the CDR-H2, and the CDR-H3 contained within SEQ ID NO: 12, and the VL region comprises the CDR-L1, the CDR-L2, and the CDR-L3 contained within SEQ ID NO: 17; or
the VH region comprises the CDR-H1, the CDR-H2, and the CDR-H3 contained within SEQ ID NO: 88, and the VL region comprises the CDR-L1, the CDR-L2, and the CDR-L3 contained within SEQ ID NO: 89.

In some of any embodiments, in the first binding domain:
the VH region comprises the CDR-H1, the CDR-H2, and the CDR-H3 comprising the amino acid sequence set forth in SEQ ID NOS: 9, 10, and 11, respectively, and the VL region comprises the CDR-L1, the CDR-L2, and the CDR-L3 comprising the amino acid sequence set forth in SEQ ID NOS: 14, 15, and 16, respectively;
the VH region comprises the CDR-H1, the CDR-H2, and the CDR-H3 comprising the amino acid sequence set forth in SEQ ID NOS: 118, 119, and 120, respectively, and the VL region comprises the CDR-L1, the CDR-L2, and the CDR-L3 comprising the amino acid sequence set forth in SEQ ID NOS: 121, 122, and 123, respectively;
the VH region comprises the CDR-H1, the CDR-H2, and the CDR-H3 comprising the amino acid sequence set forth in SEQ ID NOS:118, 119, and 124, respectively, and the VL region comprises the CDR-L1, the CDR-L2, and the CDR-L3 comprising the amino acid sequence set forth in SEQ ID NOS:125, 126, and 127, respectively;
the VH region comprises the CDR-H1, the CDR-H2, and the CDR-H3 comprising the amino acid sequence set forth in SEQ ID NOS:118, 119, and 128, respectively, and the VL region comprises the CDR-L1, the CDR-L2, and the CDR-L3 comprising the amino acid sequence set forth in SEQ ID NOS: 129, 126, and 130, respectively;
the VH region comprises the CDR-H1, the CDR-H2, and the CDR-H3 comprising the amino acid sequence set forth in SEQ ID NOS: 131, 132, and 133, respectively, and the VL region comprises the CDR-L1, the CDR-L2, and the CDR-L3 comprising the amino acid sequence set forth in SEQ ID NOS: 134, 25, and 135, respectively;
the VH region comprises the CDR-H1, the CDR-H2, and the CDR-H3 comprising the amino acid sequence set forth in SEQ ID NOS: 118, 119, and 136, respectively, and the VL region comprises the CDR-L1, the CDR-L2, and the CDR-L3 comprising the amino acid sequence set forth in SEQ ID NOS:137, 138, and 139, respectively;
the VH region comprises the CDR-H1, the CDR-H2, and the CDR-H3 comprising the amino acid sequence set forth in SEQ ID NOS:140, 141, and 142, respectively, and the VL region comprises the CDR-L1, the CDR-L2, and the CDR-L3 comprising the amino acid sequence set forth in SEQ ID NOS: 143, 144, and 145, respectively;
the VH region comprises the CDR-H1, the CDR-H2, and the CDR-H3 comprising the amino acid sequence set forth in SEQ ID NOS: 146, 119, and 147, respectively, and the VL region comprises the CDR-L1, the CDR-L2, and the CDR-L3 comprising the amino acid sequence set forth in SEQ ID NOS: 148, 149, and 150, respectively;
the VH region comprises the CDR-H1, the CDR-H2, and the CDR-H3 comprising the amino acid sequence set forth in SEQ ID NOS: 118, 119, and 151, respectively, and the VL region comprises the CDR-L1, the CDR-L2, and the CDR-L3 comprising the amino acid sequence set forth in SEQ ID NOS: 152, 126, and 153, respectively; or
the VH region comprises the CDR-H1, the CDR-H2, and the CDR-H3 comprising the amino acid sequence set forth in SEQ ID NOS:131, 154, and 155, respectively, and the VL region comprises the CDR-L1, the CDR-L2, and the CDR-L3 comprising the amino acid sequence set forth in SEQ ID NOS: 156, 157, and 158, respectively.

In some of any embodiments, in the first binding domain:
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 44, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 45;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 46, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 47;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 48, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 49;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 50, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 51;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 52, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 53;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 54, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 55;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 56, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 57;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 58, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 59;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 60, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 61;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 62, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 63;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 12, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 17; or
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 88, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 89.

In some of any embodiments, in the first binding domain:
the VH region and the VL region comprise the amino acid sequences set forth in SEQ ID NO: 32 and 33, respectively;
the VH region and the VL region comprise the amino acid sequences set forth in SEQ ID NO: 34 and 35, respectively;
the VH region and the VL region comprise the amino acid sequences set forth in SEQ ID NO: 44 and 45, respectively;
the VH region and the VL region comprise the amino acid sequences set forth in SEQ ID NO: 46 and 47, respectively;
the VH region and the VL region comprise the amino acid sequences set forth in SEQ ID NO: 48 and 49, respectively;
the VH region and the VL region comprise the amino acid sequences set forth in SEQ ID NO: 50 and 51, respectively;
the VH region and the VL region comprise the amino acid sequences set forth in SEQ ID NO: 52 and 53, respectively;
the VH region and the VL region comprise the amino acid sequences set forth in SEQ ID NO: 54 and 55, respectively;
the VH region and the VL region comprise the amino acid sequences set forth in SEQ ID NO: 56 and 57, respectively;
the VH region and the VL region comprise the amino acid sequences set forth in SEQ ID NO: 58 and 59, respectively;
the VH region and the VL region comprise the amino acid sequences set forth in SEQ ID NO: 60 and 61, respectively;
the VH region and the VL region comprise the amino acid sequences set forth in SEQ ID NO: 62 and 63, respectively;
the VH region and the VL region comprise the amino acid sequences set forth in SEQ ID NO: 12 and 17, respectively; or
the VH region and the VL region comprise the amino acid sequences set forth in SEQ ID NO: 88 and 89, respectively.

In some of any embodiments, the first binding domain is an scFv. In some of any embodiments, the first binding domain is an scFv that is a disulfide-stabilized scFv. In some of any embodiments, the scFv comprises a VH-linker-VL arrangement. In some of any embodiments, the scFv comprises a VL-linker-VH arrangement. In some of any embodiments, the linker is a peptide linker comprising (Gly4Ser)3 (SEQ ID NO: 162). In some of any embodiments, the linker is a peptide linker comprising Gly4Ser (SEQ ID NO: 160). In some of any embodiments, the linker is a peptide linker comprising (Gly4Ser)2 (SEQ ID NO: 161).

In some of any embodiments, the first binding domain comprises:
(a) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 245;
(b) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 248;
(c) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 249; or
(d) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 252.

In some of any embodiments, the first binding domain comprises: (a) the amino acid sequence of SEQ ID NO: 245; (b) the amino acid sequence of SEQ ID NO: 248; (c) the amino acid sequence of SEQ ID NO: 249; or (d) the amino acid sequence of SEQ ID NO: 252.

In some of any embodiments, in the first binding domain, the VH region comprises the CDR-H1, a CDR-H2, and a CDR-H3 comprising the amino acid sequence set forth in SEQ ID NOs: 9, 10, and 11, respectively, and the VL region comprises a CDR-L1, a CDR-L2, and a CDR-L3 comprising the amino acid sequence set forth in SEQ ID NOS: 14, 15, and 16, respectively.

In some of any embodiments, in the first binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 12, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 17. In some of any embodiments, the VH region and the VL region comprises the amino acid sequence set forth in SEQ ID NOs: 12 and 17, respectively.

In some of any embodiments, in the first binding domain, the VH region comprises the CDR-H1, a CDR-H2, and a CDR-H3 comprising the sequence set forth in SEQ ID NOs: 118, 119, and 120, respectively, and the VL region comprises a CDR-L1, a CDR-L2, and a CDR-L3 comprising the sequence set forth in SEQ ID NOS: 121, 122, 123, respectively.

In some of any embodiments, in the first binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 88, and
the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 89. In some of any embodiments, in the first binding domain, the VH region and
the VL region comprises the sequence set forth in SEQ ID NOs: 88 and 89, respectively.

In some of any embodiments, the second binding domain that binds CD3 comprises a heavy chain variable (VH) region and a light chain variable (VL) region, wherein:
the VH comprises a heavy chain complementarity determining region 1 (CDR-H1) comprising the amino acid sequence set forth in SEQ ID NO: 198, a heavy chain complementarity determining region 2 (CDR-H2) comprising the amino acid sequence set forth in SEQ ID NO: 276, and a heavy chain complementarity determining region 3 (CDR-H3) comprising the amino acid sequence set forth in SEQ ID NO: 200; and
the VL comprises a light chain complementarity determining region 1 (CDR-L1) comprising the amino acid sequence set forth in SEQ ID NO: 24, a light chain complementarity determining region 2 (CDR-L2) comprising the amino acid sequence set forth in SEQ ID NO: 201, and a light chain complementarity determining region 3 (CDR-L3) comprising the amino acid sequence set forth within SEQ ID NO: 277.

In some of any embodiments, in the second binding domain, the VH comprises a heavy chain complementarity determining region 1 (CDR-H1) comprising the amino acid sequence set forth in SEQ ID NO: 198, a heavy chain complementarity determining region 2 (CDR-H2) comprising the amino acid sequence set forth in SEQ ID NO: 199, and a heavy chain complementarity determining region 3 (CDR-H3) comprising the amino acid sequence set forth in SEQ ID NO: 200; and
the VL comprises a light chain complementarity determining region 1 (CDR-L1) comprising the amino acid sequence set forth in SEQ ID NO: 24, a light chain complementarity determining region 2 (CDR-L2) comprising the amino acid sequence set forth in SEQ ID NO: 201, and a light chain complementarity determining region 3 (CDR-L3) comprising the amino acid sequence set forth within SEQ ID NO: 106.

In some of any embodiments, in the second binding domain, the VH region binding domain and the VL region comprise, respectively: a VH region sequence of the consensus sequence set forth in QVQLVESGGGVVQPGX₁SLRLSCAASGFTFSX₂X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁L TX₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G or A or S; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 278); and a VL region sequence of the consensus sequence set forth in AIQLTQSPSSLSASVGDRVTITCRASQGISSALAWYQQKPGKAPKLLIYDASX₁LEX₂G VPSRFSGSGSGTDFTLTISSLQPEDFATYYCQQFX₃SYPRTFGX₄GTKVEIK, wherein X₁ is S or G, X₂ is N or S; X₃ is K or N; and X₄ is Q or C (SEQ ID NO: 197).

In some of any embodiments, in the second binding domain, the VH region binding domain and the VL region comprise, respectively: a VH region sequence of the consensus sequence set forth in QVQLVESGGGVVQPGX₁SLRLSCAASGFTFSX₂X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁L TX₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G or A; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 196); and a VL region sequence of the consensus sequence set forth in AIQLTQSPSSLSASVGDRVTITCRASQGISSALAWYQQKPGKAPKLLIYDASX₁LEX₂G VPSRFSGSGSGTDFTLTISSLQPEDFATYYCQQFX₃SYPRTFGX₄GTKVEIK, wherein X₁ is S or G, X₂ is N or S; X₃ is K or N; and X₄ is Q or C (SEQ ID NO: 197).

In some of any embodiments, in the second binding domain:
the CDR-H1 comprises the sequence set forth in any one of SEQ ID NOs: 19, 109, 202, or 203, the CDR-H2 comprises the sequence set forth in any one of SEQ ID NOs: 20, 107, 110, 112, or 204-208, and the CDR-H3 comprises the sequence set forth in any one of SEQ ID NOs: 21, 108, 111, 113, or 209-214; and
the CDR-L1 comprises the sequence set forth in SEQ ID NOs: 24, the CDR-L2 comprises the sequence set forth in any one of SEQ ID NOs: 25, 114, 215, or 216, and the CDR-L3 comprises the sequence set forth in any one of SEQ ID NOs: 26 or 115.

In some of any embodiments, in the second binding domain:
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NO: 37;
the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 40, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 42, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 22, and the VL region comprises the CDR-L1, the CDR-L2, and the CDR-L3 contained within SEQ ID NO: 27;
the VH region comprises the CDR-H1, the CDR-H2, and the CDR-H3 contained within SEQ ID NO: 68, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 70, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 72, and the VL region comprises the CDR-L1, the CDR-L2, and the CDR-L3 contained within SEQ ID NO: 73;
the VH region comprises the CDR-H1, the CDR-H2, and the CDR-H3 contained within SEQ ID NO: 74, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 78, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 80, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 82, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 84, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 86, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 159, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 195, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 41, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 64, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 65, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 66, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 67, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 69, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 71, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 75, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 76, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 77, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 79, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 81, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 83, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 85, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 87, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 90, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 91, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 92, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 93, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 94, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 95, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 96, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 97, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 98, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 39;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 99;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 43;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 100;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 101;
the VH region comprises the CDR-H1, the CDR-H2, and the CDR-H3 contained within SEQ ID NO: 42, and the VL region comprises the CDR-L1, the CDR-L2, and the CDR-L3 contained within SEQ ID NOs: 37;
the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NOs: 37; or
the VH region comprises the CDR-H1, the CDR-H2, and the CDR-H3 contained within SEQ ID NO: 40, and the VL region comprises the CDR-L1, the CDR-L2, and the CDR-L3 contained within SEQ ID NOs: 37.

In some of any embodiments, in the second binding domain:
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 109, 110, and 108, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 110, and 21, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 20, and 21, respectively, and the VL region comprises
the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 20, and 111, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 112, and 21, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 20, and 113, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 204, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 205, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 206, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 209, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 113, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 210, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 211, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 111, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 109, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 202, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 203, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 212, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 213, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 214, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 21, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 207, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 208, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 110, and 113, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 110, and 210, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 202, 110, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 110, and 214, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 115, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 215, and 26, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 216, and 115, respectively;
the VH region comprises the CDR-H1, the CDR-H2, and the CDR-H3 comprising the sequence set forth in SEQ ID NOS: 19, 110, and 111, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 109, 110, 108, respectively, and the VL region comprises
the CDR-L1, the CDR-L2, and the CDR-L3 comprising the sequence set forth in SEQ ID NOS: 24, 114, and 26, respectively; or
the VH region comprises the CDR-H1, the CDR-H2, and the CDR-H3 comprising the sequence set forth in SEQ ID NOS: 19, 110, and 21, respectively, and the VL region comprises the CDR-L1, the CDR-L2, and the CDR-L3 comprising the sequence set forth in SEQ ID NOS: 24, 114, and 26, respectively.

In some of any embodiments, wherein in the second binding domain:
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 22, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 68, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 70, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 72, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 73;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 74, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 78, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 80, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 82, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 84, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 86, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 159, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 195, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 64, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 65, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 66, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 67, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 69, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 71, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 75, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 76, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 77, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 79, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 81, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 83, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 85, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 87, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 90, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 91, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 92, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 93, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 94, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 95, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 96, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 97, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 98, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 99;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 100;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 101;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37; or
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37.

In some of any embodiments, in the second binding domain:
the VH region and the VL region comprise the sequences set forth in SEQ ID NO: 36 and 37, respectively;
the VH region and the VL region comprise the sequences set forth in SEQ ID NO: 38 and 39, respectively;
the VH region and the VL region comprise the sequences set forth in SEQ ID NO: 40 and 39, respectively;
the VH region and the VL region comprise the sequences set forth in SEQ ID NO: 42 and 43, respectively;
the VH region and the VL region comprise the sequences set forth in SEQ ID NO: 22 and 27, respectively;
the VH region and the VL region comprise the sequences set forth in SEQ ID NO: 68 and 39, respectively;
the VH region and the VL region comprise the sequences set forth in SEQ ID NO: 70 and 43, respectively;
the VH region and the VL region comprise the sequences set forth in SEQ ID NO: 72 and 73, respectively;
the VH region and the VL region comprise the sequences set forth in SEQ ID NO: 74 and 39, respectively;
the VH region and the VL region comprise the sequences set forth in SEQ ID NO: 78 and 39, respectively;
the VH region and the VL region comprise the sequences set forth in SEQ ID NO: 80 and 39, respectively;
the VH region and the VL region comprise the sequences set forth in SEQ ID NO: 82 and 39, respectively;
the VH region and the VL region comprise the sequences set forth in SEQ ID NO: 84 and 39, respectively;
the VH region and the VL region comprise the sequences set forth in SEQ ID NO: 86 and 39, respectively;
the VH region and the VL region comprise the sequences set forth in SEQ ID NO: 159 and 43, respectively;
the VH region and the VL region comprise the sequences set forth in SEQ ID NO: 195 and 39, respectively;
the VH region and the VL region comprise the sequences set forth in SEQ ID NO: 41 and 37, respectively;
the VH region and the VL region comprise the sequences set forth in SEQ ID NO: 64 and 37, respectively;
the VH region and the VL region comprise the sequences set forth in SEQ ID NO: 65 and 37, respectively;
the VH region and the VL region comprise the sequences set forth in SEQ ID NO: 66 and 37, respectively;
the VH region and the VL region comprise the sequences set forth in SEQ ID NO: 67 and 37, respectively;
the VH region and the VL region comprise the sequences set forth in SEQ ID NO: 69 and 37, respectively;
the VH region and the VL region comprise the sequences set forth in SEQ ID NO: 71 and 37, respectively;
the VH region and the VL region comprise the sequences set forth in SEQ ID NO: 75 and 37, respectively;
the VH region and the VL region comprise the sequences set forth in SEQ ID NO: 76 and 37, respectively;
the VH region and the VL region comprise the sequences set forth in SEQ ID NO: 77 and 37, respectively;
the VH region and the VL region comprise the sequences set forth in SEQ ID NO: 79 and 37, respectively;
the VH region and the VL region comprise the sequences set forth in SEQ ID NO: 81 and 37, respectively;
the VH region and the VL region comprise the sequences set forth in SEQ ID NO: 83, and 37, respectively;
the VH region and the VL region comprise the sequences set forth in SEQ ID NO: 85 and 37, respectively;
the VH region and the VL region comprise the sequences set forth in SEQ ID NO: 87 and 37, respectively;
the VH region and the VL region comprise the sequences set forth in SEQ ID NO: 90 and 37, respectively;
the VH region and the VL region comprise the sequences set forth in SEQ ID NO: 91 and 37, respectively;
the VH region and the VL region comprise the sequences set forth in SEQ ID NO: 92 and 37, respectively;
the VH region and the VL region comprise the sequences set forth in SEQ ID NO: 93 and 37, respectively;
the VH region and the VL region comprise the sequences set forth in SEQ ID NO: 94 and 37, respectively;
the VH region and the VL region comprise the sequences set forth in SEQ ID NO: 95 and 37, respectively;
the VH region and the VL region comprise the sequences set forth in SEQ ID NO: 96 and 37, respectively;
the VH region and the VL region comprise the sequences set forth in SEQ ID NO: 97 and 37, respectively;
the VH region and the VL region comprise the sequences set forth in SEQ ID NO: 98 and 37, respectively;
the VH region and the VL region comprise the sequences set forth in SEQ ID NO: 36 and 39, respectively;
the VH region and the VL region comprise the sequences set forth in SEQ ID NO: 36 and 99, respectively;
the VH region and the VL region comprise the sequences set forth in SEQ ID NO: 36 and 43, respectively;
the VH region and the VL region comprise the sequences set forth in SEQ ID NO: 36 and 100, respectively;
the VH region and the VL region comprise the sequences set forth in SEQ ID NO: 36 and 101, respectively;
the VH region and the VL region comprise the sequences set forth in SEQ ID NO: 42 and 37, respectively;
the VH region and the VL region comprise the sequences set forth in SEQ ID NO: 38 and 37, respectively;
the VH region and the VL region comprise the sequences set forth in SEQ ID NO: 40 and 37, respectively.

In some of any embodiments, in the second binding domain:
the VH region comprising a sequence of the consensus sequence QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTX₈X₉YX₁₀X₁₁GX₁₂X₁₃VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is R or G; X₉ is Y or H; X₁₀ is Q or S; X₁₁ is Y or H; X₁₂ is I or L; and X₁₃ is N or D; (SEQ ID NO: 116); and
the VL region comprising a sequence of the consensus sequence set forth in AIQLTQSPSSLSASVGDRVTITCRASQGISSALAWYQQKPGKAPKLLIYDASSLEX₁GV PSRFSGSGSGTDFTLTISSLQPEDFATYYCQQFX₂SYPRTFGX₃GTKVEIK, wherein X₁ is N or S; X₂ is K or N; and X₃ is Q or C; (SEQ ID NO: 117).

In some of any embodiments, in the second binding domain:
the VH region comprising a sequence of the consensus sequence QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTRYY X₈X₉GX₁₀X₁₁VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is Q or S; X₉ is Y or H; X₁₀ is I or L; and X₁₁ is N or D; (SEQ ID NO: 192); and the VL region comprising a sequence of the consensus sequence set forth in AIQLTQSPSSLSASVGDRVTITCRASQGISSALAWYQQKPGKAPKLLIYDASSLESGVP SRFSGSGSGTDFTLTISSLQPEDFATYYCQQFX₁SYPRTFGX₂GTKVEIK, wherein X₁=K or N
X₂=Q or C; (SEQ ID NO: 193).

In some of any embodiments, in the second binding domain, the VH comprises one or more amino acid residues selected from the group consisting of: an asparagine (N) at position 31, an alanine (A) at position 55, a tyrosine (Y) at position 80, a glycine (G) at position 106, a histidine (H) at position 107, a serine (S) at position 109, a histidine (H) at position 110, a leucine (L) at position 112, and an aspartic acid (D) at position 113 or any combination thereof, wherein the numbering is relative to SEQ ID NO: 36.

In some of any embodiments, in the second binding domain, the VH comprises:
(a) amino acid residues asparagine (N) at position 31, alanine (A) at position 55, and tyrosine (Y) at position 80, wherein the numbering is relative to SEQ ID NO: 36;
(b) alanine (A) at position 55, tyrosine (Y) at position 80, leucine (L) at position 112, and aspartic acid (D) at position 113, wherein the numbering is relative to SEQ ID NO: 36; or
(c) alanine (A) at position 55, tyrosine (Y) at position 80, serine (S) at position 109, and histidine (H) at position 110, wherein the numbering is relative to SEQ ID NO: 36;

In some of any embodiments, in the second binding domain, the VL region comprises one or more amino acid residues selected from the group consisting of: a serine (S) at position 56, an asparagine (N) at position 92, and a cysteine (C) at position 100, an asparagine (N) at position 108, or any combination thereof in relation to SEQ ID NO: 37. In some of any embodiments, in the second binding domain, the VL region comprises: (a) serine (S) at position 56, wherein the numbering is relative to SEQ ID NO: 36; or (b) serine (S) at position 56 and asparagine (N) at position 92, wherein the numbering is relative to SEQ ID NO: 37.

In some of any embodiments, in the second binding domain, the VH comprises one or more amino acid residues selected from the group consisting of: a glycine (G) at position 16, a glutamic acid (E) at position 16, a threonine (T) at position 58, an aspartic acid (D) at position 58, a glutamic acid (E) at position 76, a serine (S) at position 76, or any combination thereof in relation to SEQ ID NO: 36.

In some of any embodiments, the second binding domain is an antigen binding antibody fragment. In some embodiments, the second binding domain is a scFv, is a Fab, or is an Fv. In some embodiments, the second binding domain is an scFv. In some of any embodiments, the second binding domain is a Fab. In some of any embodiments, the second binding domain is an scFv that is a disulfide-stabilized scFv. In some of any embodiments, the scFv comprises a VH-linker-VL arrangement. In some of any embodiments, the scFv comprises a VL-linker-VH arrangement. In some of any embodiments, the scFv comprises a peptide linker comprising (Gly4Ser)3 (SEQ ID NO: 162). In some of any embodiments, the linker is a peptide linker comprising Gly4Ser (SEQ ID NO: 160). In some of any embodiments, the linker is a peptide linker comprising (Gly4Ser)2 (SEQ ID NO: 161).

In some of any embodiments, in the second binding domain, the VH region comprises cysteine (C) at position 44 and the VL region comprises a cysteine (C) at position 100 in relation to SEQ ID NO: 36.

In some of any embodiments, in the second binding domain, the VH region comprises the CDR-H1, the CDR-H2, and the CDR-H3 contained within any one of SEQ ID NOs: 36, 38, 40, 42, 22, 68, 70, 72, 74, 78, 80, 82, 84, 86, 159 or 195, and the VL region comprises the CDR-L1, the CDR-L2, and the CDR-L3 contained within any one of SEQ ID NOs: 27, 37, 39, 43, or 73. In some of any embodiments, in the second binding domain:
the CDR-H1 comprises the sequence set forth in any one of SEQ ID NOs: 19, or 109, the CDR-H2 comprises the sequence set forth in any one of SEQ ID NOs: 20, 107, 110, or 112, and the CDR-H3 comprises the sequence set forth in any one of SEQ ID NOs: 21, 108, 111, or 113; and
the CDR-L1 comprises the sequence set forth in SEQ ID NOs: 24, the CDR-L2 comprises the sequence set forth in any one of SEQ ID NOs: 25 or 114, and the CDR-L3 comprises the sequence set forth in any one of SEQ ID NOs: 26 or 115.

In some of any embodiments, in the second binding domain:
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NO: 37;
the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 40, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 42, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 22, and the VL region comprises the CDR-L1, the CDR-L2, and the CDR-L3 contained within SEQ ID NO: 27;
the VH region comprises the CDR-H1, the CDR-H2, and the CDR-H3 contained within SEQ ID NO: 68, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 70, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 72, and the VL region comprises the CDR-L1, the CDR-L2, and the CDR-L3 contained within SEQ ID NO: 73;
the VH region comprises the CDR-H1, the CDR-H2, and the CDR-H3 contained within SEQ ID NO: 74, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 78, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 80, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 82, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 84, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 86, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 159, and the VL region comprises the CDR-L1, the CDR-L2, and the CDR-L3 contained within SEQ ID NO: 43; or
the VH region comprises the CDR-H1, the CDR-H2, and the CDR-H3 contained within SEQ ID NO: 195, and the VL region comprises the CDR-L1, the CDR-L2, and the CDR-L3 contained within SEQ ID NO: 39.

In some of any embodiments, in the second binding domain:
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 109, 110, and 108, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 110, and 21, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 20, and 21, respectively, and the VL region comprises
the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 20, and 111, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 112, and 21, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 20, and 113, respectively, and the VL region comprises the CDR-L1, the CDR-L2, and the CDR-L3 comprising the sequence set forth in SEQ ID NOS: 24, 25, and 115, respectively.

In some of any embodiments, in the second binding domain:
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 22, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 68, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 70, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 72, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 73;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 74, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 78, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 80, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 82, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 84, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 86, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 159, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43; or
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 195, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39.

In some of any embodiments, in the second binding domain:
the VH region and the VL region comprise the sequences set forth in SEQ ID NO: 36 and 37, respectively;
the VH region and the VL region comprise the sequences set forth in SEQ ID NO: 38 and 39, respectively;
the VH region and the VL region comprise the sequences set forth in SEQ ID NO: 40 and 39, respectively;
the VH region and the VL region comprise the sequences set forth in SEQ ID NO: 42 and 43, respectively;
the VH region and the VL region comprise the sequences set forth in SEQ ID NO: 22 and 27, respectively;
the VH region and the VL region comprise the sequences set forth in SEQ ID NO: 68 and 39, respectively;
the VH region and the VL region comprise the sequences set forth in SEQ ID NO: 70 and 43, respectively;
the VH region and the VL region comprise the sequences set forth in SEQ ID NO: 72 and 73, respectively;
the VH region and the VL region comprise the sequences set forth in SEQ ID NO: 74 and 39, respectively;
the VH region and the VL region comprise the sequences set forth in SEQ ID NO: 78 and 39, respectively;
the VH region and the VL region comprise the sequences set forth in SEQ ID NO: 80 and 39, respectively;
the VH region and the VL region comprise the sequences set forth in SEQ ID NO: 82 and 39, respectively;
the VH region and the VL region comprise the sequences set forth in SEQ ID NO: 84 and 39, respectively;
the VH region and the VL region comprise the sequences set forth in SEQ ID NO: 86 and 39, respectively;
the VH region and the VL region comprise the sequences set forth in SEQ ID NO: 159 and 43, respectively; or
the VH region and the VL region comprise the sequences set forth in SEQ ID NO: 195 and 39, respectively; or.

In some of any embodiments, the second binding domain is an scFv comprising:
(a) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 246;
(b) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 247;
(c) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 250; or
(d) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 251.

In some of any embodiments, the second binding domain is an scFv comprising: (a) the amino acid sequence of SEQ ID NO: 246; (b) the amino acid sequence of SEQ ID NO: 247; (c) the amino acid sequence of SEQ ID NO: 250; or (d) the amino acid sequence of SEQ ID NO: 251. In some of any embodiments, in the second binding domain, the VH region comprises the CDR-H1, the CDR-H2, and the CDR-H3 comprising the sequence set forth in SEQ ID NOs: 19, 20, and 21, respectively, and the VL region comprises the CDR-L1, the CDR-L2, and the CDR-L3 comprising the sequence set forth in SEQ ID NOS: 24, 25, and 26, respectively.

In some of any embodiments, in the second binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 22, and the VL region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 27. In some of any embodiments, in the second binding domain, the VH region and the VL region comprises the sequence or are the sequences set forth in SEQ ID NO: 22 and 27, respectively.

In some of any embodiments, in the second binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 68, and the VL region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 39. In some of any embodiments, in the second binding domain, the VH region and the VL region comprise the sequences or are the sequences set forth in SEQ ID NO: 68 and 39, respectively.

In some of any embodiments, in the second binding domain, the VH region comprises the CDR-H1, the CDR-H2, and the CDR-H3 comprising the sequence set forth in SEQ ID NOs: 19, 20, and 111, respectively, and the VL region comprises the CDR-L1, the CDR-L2, and the CDR-L3 comprising the sequence set forth in SEQ ID NOS: 24, 25, and 115, respectively. In some of any embodiments, in the second binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 70, and the VL region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 43. In some of any embodiments, in the CD3 binding domain, the VH region and the VL region comprise the sequences or are the sequences set forth in SEQ ID NO: 70 and 43, respectively.

In some of any embodiments, in the second binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 72, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 73. In some of any embodiments, in the second binding domain, the VH region and the VL region comprise the sequences or are the sequences set forth in SEQ ID NO: 72 and 73, respectively.

In some of any embodiments, the first binding domain comprises the VH set forth in any one of SEQ ID NOs: 12 or 88 and the VL set forth in any one of SEQ ID NO: 17 or 89; and the second binding domain comprises the VH set forth in any one of SEQ ID NOs: 22, 68, 72, or 70 and the VL set forth in any one of SEQ ID NO: 27, 39, 73, or 43. In some of any embodiments, the first binding domain comprises the VH set forth in SEQ ID NO: 12 and the VL set forth in SEQ ID NO: 17; and the second binding domain comprises the VH set forth in SEQ ID NO: 22 and the VL set forth in SEQ ID NO: 27. In some of any embodiments, the first binding domain comprises the VH set forth in SEQ ID NO: 12 and the VL set forth in SEQ ID NO: 17; and the second binding domain comprises the VH set forth in SEQ ID NO: 68 and the VL set forth in SEQ ID NO: 39.

In some of any embodiments, the first binding domain comprises the VH set forth in SEQ ID NO: 12 and the VL set forth in SEQ ID NO: 17; and the CD3 binding domain comprises the VH set forth in SEQ ID NO: 72 and the VL set forth in SEQ ID NO: 73. In some of any embodiments, the first binding domain comprises the VH set forth in SEQ ID NO: 12 and the VL set forth in SEQ ID NO: 17; and the second binding domain comprises the VH set forth in SEQ ID NO: 70 and the VL set forth in SEQ ID NO: 43.

In some of any embodiments, the first binding domain comprises the VH set forth in SEQ ID NO: 88 and the VL set forth in SEQ ID NO: 89; and the second binding domain comprises the VH set forth in SEQ ID NO: 22 and the VL set forth in SEQ ID NO: 27. In some of any embodiments, the first binding domain comprises the VH set forth in SEQ ID NO: 88 and the VL set forth in SEQ ID NO: 89; and the second binding domain comprises the VH set forth in SEQ ID NO: 68 and the VL set forth in SEQ ID NO: 39. In some of any embodiments, the first binding domain comprises the VH set forth in SEQ ID NO: 88 and the VL set forth in SEQ ID NO: 89; and the second binding domain comprises the VH set forth in SEQ ID NO: 72 and the VL set forth in SEQ ID NO: 73. In some of any embodiments, the first binding domain comprises the VH set forth in SEQ ID NO: 88 and the VL set forth in SEQ ID NO: 89; and the second binding domain comprises the VH set forth in SEQ ID NO: 70 and the VL set forth in SEQ ID NO: 43.

In some of any embodiments, the second binding domain has a KD of 5 to 75 nM to CD3. In some of any embodiments, the second binding domain has a KD of 10 to 70 nM to CD3.

In some of any embodiments, the first binding domain and the second binding domain are independently selected from a scFv and a Fab. In some of any embodiments, the first binding domain and/or the second binding domain is a scFv. In some of any embodiments, the first and the second binding domain are both an scFv. In some of any embodiments, the first binding domain is a scFv and the second binding domain is a Fab.

In some of any embodiments, the first binding domain comprises, from N- to C-terminal order: the VH region and the VL region, optionally separated by a linker. In some of any embodiments, the first binding domain comprises, from N- to C-terminal order: the VL region and the VH region, optionally separated by a linker. In some of any embodiments, the first binding domain is a scFv that is disulfide-stabilized such that the VH region and VL region are covalently linked by a disulfide bond, optionally wherein the VH region comprises a cysteine residue at framework region 2 position 44 (Kabat numbering) and the VL region comprises a cysteine residue at framework region 4 position 100 (Kabat numbering).

In some of any embodiments, the first binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 245. In some of any embodiments, the first binding domain comprises the amino acid sequence of SEQ ID NO: 245. In some of any embodiments, the first binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 248. In some of any embodiments, the first binding domain comprises the amino acid sequence of SEQ ID NO: 248. In some of any embodiments, the first binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 249. In some of any embodiments, the first binding domain comprises the amino acid sequence of SEQ ID NO: 249. In some of any embodiments, the first binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 252. In some of any embodiments, the first binding domain comprises the amino acid sequence of SEQ ID NO: 252.

In some of any embodiments, the second binding domain comprises, from N- to C-terminal order: the VH region and the VL region, optionally separated by a linker. In some of any embodiments, the second binding domain comprises, from N- to C-terminal order: the VL region and the VH region, optionally separated by a linker. In some of any embodiments, the second binding domain is a scFv that is disulfide-stabilized such that the VH region and VL region are covalently linked by a disulfide bond, optionally wherein the VH region comprises a cysteine residue at framework region 2 position 44 (Kabat numbering) and the VL region comprises a cysteine residue at framework region 4 position 100 (Kabat numbering).

In some of any embodiments, the second binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 246. In some of any embodiments, the second binding domain comprises the amino acid sequence of SEQ ID NO: 246. In some of any embodiments, the second binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 247. In some of any embodiments, the second binding domain comprises the amino acid sequence of SEQ ID NO: 247.

In some of any embodiments, the antibody comprises a constant region from an IgG1 or IgG2. In some of any embodiments, the constant region is a human IgG1 heavy chain constant region. In some of any embodiments, the antibody comprises an Fc region comprising a first and second Fc polypeptide each comprising a hinge-CH2-CH3. In some of any embodiments, the first binding domain and the second binding domain are together present on two polypeptide chains.

In certain embodiments, the constant region comprises the amino acid sequence set forth in any of SEQ ID NOs: 163-166 and 169.

In certain embodiments, the constant region of one heavy chain comprises an amino acid sequence set forth in SEQ ID NOs: 163-166 and the constant region of the other heavy chain independently comprises an amino acid sequence set forth in SEQ ID NOs: 163-166.

In certain embodiments, the constant region of one heavy chain comprises the amino acid sequence set forth in SEQ ID NO: 165 and the constant region of the other heavy chain comprises the amino acid sequence set forth in SEQ ID NO: 166.

In certain embodiments, the constant region is a human IgG2 heavy chain constant region.

In certain embodiments, the constant region comprises the amino acid sequence set forth in any of SEQ ID NO: 167, 168, 170.

In some of any embodiments, the bispecific antibody comprises a first heavy chain and a second heavy chain, wherein:
(a) the first heavy chain comprises (i) the first binding domain that is an scFv comprising the VH region and the VL region and (ii) a first Fc polypeptide comprising a hinge-CH2-CH3; and
(b) the second heavy chain comprises (i) the second binding domain that is an scFv comprising the VH region and the VL region and (ii) a second Fc polypeptide comprising a hinge-CH2-CH3.

In some of any embodiments, the first binding domain and the second binding domain are together present on three polypeptide chains. In some of any embodiments, the bispecific antibody comprises a first heavy chain, a second heavy chain, and a light chain, wherein:
(a) the first heavy chain comprises (i) the first binding domain that is an scFv comprising the VH region and the VL region and (ii) a first Fc polypeptide comprising a hinge-CH2-CH3;
(b) the second heavy chain comprises (i) the heavy chain of the second binding domain that is the heavy chain of a Fab comprising the VH region and CH1, (ii) and a second Fc polypeptide comprising a hinge-CH2-CH3; and
(c) a light chain comprises the light chain of the second binding domain that is the light chain of the Fab comprising the VL region and the constant light chain (CL).

In some of any embodiments, the first binding domain and the second binding domain are each N-terminal to the Fc region. In some of any embodiments, the Fc region is a heterodimeric Fc region. In some of any embodiments, each of the first and second polypeptide comprises one or more amino acid substitutions in a wild-type Fc polypeptide region to effect heterodimer formation between the first polypeptide and the second polypeptide. In some embodiments, the wild-type Fc region is an IgG1 Fc region or an IgG2 Fc region, optionally wherein the wild-type Fc region is a wildtype IgG1 Fc region comprising the amino acid sequence set forth in SEQ ID NO: 253.

In some of any embodiments, the one more amino acid substitutions are a knob-into-hole modification, a charge mutation to reduce or prevent self-association due to charge repulsion, or disulfide bonding pairing mutation, or combinations thereof. In some of any embodiments, the one or more amino acid substitutions are a knob-into-hole modification and disulfide bond pairing mutation.

In some of any embodiments, the one or more amino acid substitutions are:
(i) amino acid substitution T366W in the first polypeptide of the heterodimeric Fc and amino acid substitutions T366S, L368A and Y407V in the second polypeptide of the heterodimeric Fc; and/or
(ii) amino acid substitution S354C on the first polypeptide of the heterodimeric Fc and amino acid substitution Y349C on the second polypeptide of the heterodimeric Fc.

In some of any embodiments, the heterodimeric Fc region comprises one or more amino acid substitutions to reduce binding affinity to an Fc receptor and/or to reduce effector function, optionally as compared to a wild-type IgG1 Fc region. In some embodiments, the wild-type Fc region is a wild-type IgG1 Fc region comprising the amino acid sequence set forth in SEQ ID NO: 253. In some of any embodiments, the one or more amino acid substitutions are selected from L234A, L234V, L235A, L235E, G237A, D265S, S267K, R292C, N297G, V302C, and P329G by EU numbering. In some of any embodiments, the one or more amino acid substitution comprises L234A and L235A. In some of any embodiments, the one or more amino acid substitution comprises L234A, L235A, and D265S. In some of any embodiments, the first and second Fc polypeptide is of the human IgG1 G1m1,17 allotype. In some of any embodiments, the bispecific antibody comprises the amino acid substitutions D356E and L358M. In some of any embodiments, the first Fc polypeptide is an IgG1 Fc comprising amino acid substitutions L234A, L235A, D265S, S354C and T366W, and the second Fc polypeptide is an IgG1 Fc comprising amino acid substitutions L234A, L235A, D265S, Y349C, T366S, L368A and Y407V.

In some of any embodiments, the first Fc polypeptide comprises an amino acid sequence having at least at or about 85%, 86%, 87%, 88%, 89%, 90%, 91%, 92%, 93%, 94%, 95%, 96%, 97%, 98% or 99% sequence identity SEQ ID NO:163 or 165; and the second Fc polypeptide comprises an amino acid sequence having at least at or about 85%, 86%, 87%, 88%, 89%, 90%, 91%, 92%, 93%, 94%, 95%, 96%, 97%, 98% or 99% sequence identity SEQ ID NO: 164 or 166. In some of any embodiments, the first Fc polypeptide comprises the amino acid sequence set forth in SEQ ID NO: 164 or 166; and the second Fc polypeptide comprises the amino acid sequence set forth in SEQ ID NO: 163 or 165.

In some of any embodiments, first Fc polypeptide of the heterodimeric Fc region comprises the amino acid sequence set forth in SEQ ID NO: 164 and the second Fc polypeptide of the heterodimeric Fc region comprises the amino acid sequence set forth in SEQ ID NO: 163. In some of any embodiments, the first Fc polypeptide of the heterodimeric Fc region comprises the amino acid sequence set forth in SEQ ID NO: 166 and the second Fc polypeptide of the heterodimeric Fc region comprises the amino acid sequence set forth in SEQ ID NO:165.

In some of any embodiments, the one or more amino acid substitutions are selected from P238K. In some of any embodiments, the first Fc polypeptide is an IgG2 Fc comprising amino acid substitutions P238K, D356E, L358M, Y349C, T366S, L368A and Y407V, and the second Fc polypeptide is an IgG2 Fc comprising amino acid substitutions P238K, D356E, L358M, S354C and T366W.

In some of any embodiments, the first Fc polypeptide comprises an amino acid sequence having at least at or about 85%, 86%, 87%, 88%, 89%, 90%, 91%, 92%, 93%, 94%, 95%, 96%, 97%, 98% or 99% sequence identity SEQ ID NO:168; and the second Fc polypeptide comprises an amino acid sequence having at least at or about 85%, 86%, 87%, 88%, 89%, 90%, 91%, 92%, 93%, 94%, 95%, 96%, 97%, 98% or 99% sequence identity SEQ ID NO: 167. In some of any embodiments, the first Fc polypeptide of the heterodimeric Fc region comprises the amino acid sequence set forth in SEQ ID NO: 168 and the second Fc polypeptide of the heterodimeric Fc region comprises the amino acid sequence set forth in SEQ ID NO:167.

In some of any embodiments, the bispecific antibody is capable of binding CD19 on B cells and CD3 on T cells. In some of any embodiments, the bispecific antibody results in B cell and/or plasma blast depletion of greater than 90% in the lymph node, bone marrow, and/or blood as determined by an in vitro cell depletion assay.

Provided herein is a bispecific antibody comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and
(b) a second heavy chain polypeptide comprising from N- to C-terminal order: a VH region comprising the sequence set forth in SEQ ID NO: 22, a linker of SEQ ID NO: 162, a VL region comprising the sequence set forth in SEQ ID NO: 27, and a Fc region comprising the sequence set forth in SEQ ID NO:165.

In some embodiments, the first heavy chain polypeptide comprises from N to C-terminal order the scFv comprising the sequence set forth in SEQ ID NO:245 and the Fc region comprising the sequence set forth in SEQ ID NO:166; and the second heavy chain polypeptide comprises from N to C-terminal order the scFv comprising the sequence set forth in SEQ ID NO:246 and the Fc region comprising the sequence set forth in SEQ ID NO: 165.

In some of any embodiments, the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 172; and
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 173.

In some of any embodiments, (a) the first heavy chain polypeptide comprises the amino acid sequence of SEQ ID NO: 172; and (b) the second heavy chain polypeptide comprises the amino acid sequence of SEQ ID NO: 173.

Provided herein is a bispecific antibody, comprising: (a) a first polypeptide comprising the amino acid sequence of SEQ ID NO: 172; and (b) a second polypeptide comprising the amino acid sequence of SEQ ID NO: 173.

Provided herein is a bispecific antibody, consisting essentially of: (a) a first polypeptide comprising the amino acid sequence of SEQ ID NO: 172; and (b) a second polypeptide comprising the amino acid sequence of SEQ ID NO: 173.

Provided herein is a bispecific antibody, consisting of: (a) a first polypeptide comprising the amino acid sequence of SEQ ID NO: 172; and (b) a second polypeptide comprising the amino acid sequence of SEQ ID NO: 173.

Provided herein is a bispecific antibody comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and
(b) a second heavy chain polypeptide comprising from N- to C-terminal order: a Fab comprising a V_{H} region comprising the sequence set forth in SEQ ID NO: 68 and an IgG1 CH1 of SEQ ID NO: 169, and a Fc region comprising the sequence set forth in SEQ ID NO:165; and
(c) a light chain comprises a VL region comprising the sequence set forth in SEQ ID NO: 39 and a CL comprising the sequence set forth in SEQ ID NO: 171.

In some embodiments, the first heavy chain polypeptide comprises from N to C-terminal order the scFv comprising the sequence set forth in SEQ ID NO:245 and the Fc region comprising the sequence set forth in SEQ ID NO:166. In some of any embodiments, the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 174;
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 175; and
(c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 176.

In some of any embodiments, (a) the first heavy chain polypeptide comprises the amino acid sequence of SEQ ID NO: 174; (b) the second heavy chain polypeptide comprises the amino acid sequence of SEQ ID NO: 175; and (c) the light chain comprises the amino acid sequence of SEQ ID NO: 176.

Provided herein is a bispecific antibody, comprising: (a) a first polypeptide comprising the amino acid sequence of SEQ ID NO: 174; (b) a second polypeptide comprising the amino acid sequence of SEQ ID NO: 175; and (c) a third polypeptide comprising the amino acid sequence of SEQ ID NO: 176.

Provided herein is a bispecific antibody, consisting essentially of: (a) a first polypeptide comprising the amino acid sequence of SEQ ID NO: 174; (b) a second polypeptide comprising the amino acid sequence of SEQ ID NO: 175; and (c) a third polypeptide comprising the amino acid sequence of SEQ ID NO: 176.

Provided herein is a bispecific antibody, consisting of: (a) a first polypeptide comprising the amino acid sequence of SEQ ID NO: 174; (b) a second polypeptide comprising the amino acid sequence of SEQ ID NO: 175; and (c) a third polypeptide comprising the amino acid sequence of SEQ ID NO: 176.

Provided herein is a bispecific antibody, comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and
(b) a second heavy chain polypeptide comprising from N- to C-terminal order: a VH region comprising the sequence set forth in SEQ ID NO: 72, a linker of SEQ ID NO: 162, a VL region comprising the sequence set forth in SEQ ID NO: 73, and a Fc region comprising the sequence set forth in SEQ ID NO:165.

In some embodiments, the first heavy chain polypeptide comprises from N to C-terminal order the scFv comprising the sequence set forth in SEQ ID NO:245 and the Fc region comprising the sequence set forth in SEQ ID NO:166; and the second heavy chain polypeptide comprises from N to C-terminal order the scFv comprising the sequence set forth in SEQ ID NO:247 and the Fc region comprising the sequence set forth in SEQ ID NO: 165.

In some of any embodiments, the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 177; and (b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 178. In some embodiments, (a) the first heavy chain polypeptide comprises the amino acid sequence of SEQ ID NO: 177; and (b) the second heavy chain polypeptide comprises the amino acid sequence of SEQ ID NO: 178.

Provided herein is a bispecific antibody, comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and (b) a second heavy chain polypeptide comprising from N- to C-terminal order: a Fab comprising a VH region comprising the sequence set forth in SEQ ID NO: 70 and an IgG1 CH1 of SEQ ID NO: 169, and a Fc region comprising the sequence set forth in SEQ ID NO:165; and (c) a light chain comprises a VL region comprising the sequence set forth in SEQ ID NO: 43 and a CL comprising the sequence set forth in SEQ ID NO: 171.

In some embodiments, the first heavy chain polypeptide comprises from N to C-terminal order the scFv comprising the sequence set forth in SEQ ID NO:245 and the Fc region comprising the sequence set forth in SEQ ID NO:166.

In some of any embodiments, the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 179;
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 180; and
(c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 181.

In some embodiments, (a) the first heavy chain polypeptide comprises the amino acid sequence of SEQ ID NO: 179; (b) the second heavy chain polypeptide comprises the amino acid sequence of SEQ ID NO: 180; and (c) the light chain comprises the amino acid sequence of SEQ ID NO: 181.

Provided herein is a bispecific antibody, comprising:
(a) a first heavy chain polypeptide comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and
(b) a second heavy chain polypeptide comprising from N- to C-terminal order: a VH region comprising the sequence set forth in SEQ ID NO: 22, a linker of SEQ ID NO: 162, a VL region comprising the sequence set forth in SEQ ID NO: 27, and a Fc region comprising the sequence set forth in SEQ ID NO:165.

In some embodiments, the first heavy chain polypeptide comprises from N to C-terminal order the scFv comprising the sequence set forth in SEQ ID NO:248 and the Fc region comprising the sequence set forth in SEQ ID NO:166; and the second heavy chain polypeptide comprises from N to C-terminal order the scFv comprising the sequence set forth in SEQ ID NO:246 and the Fc region comprising the sequence set forth in SEQ ID NO: 165.

In some of any embodiments, the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 182; and (b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 183. In some of any embodiments, (a) the first heavy chain polypeptide comprises the amino acid sequence of SEQ ID NO: 182; and (b) the second heavy chain polypeptide comprises the amino acid sequence of SEQ ID NO: 183.

Provided herein is a bispecific antibody, comprising:
(a) a first heavy chain polypeptide comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and (b) a second heavy chain polypeptide comprising from N- to C-terminal order: a Fab comprising a V_{H} region comprising the sequence set forth in SEQ ID NO: 68 and an IgG1 CH1 of SEQ ID NO: 169, and a Fc region comprising the sequence set forth in SEQ ID NO:165; and (c) a light chain comprises a VL region comprising the sequence set forth in SEQ ID NO: 39 and a CL comprising the sequence set forth in SEQ ID NO: 171.

In some embodiments, the first heavy chain polypeptide comprises from N to C-terminal order the scFv comprising the sequence set forth in SEQ ID NO:248 and the Fc region comprising the sequence set forth in SEQ ID NO:166.

In some embodiments, the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 184; (b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 185; and (c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 186. In some of any embodiments, (a) the first heavy chain polypeptide comprises the amino acid sequence of SEQ ID NO: 184; (b) the second heavy chain polypeptide comprises the amino acid sequence of SEQ ID NO: 185; and (c) the light chain comprises the amino acid sequence of SEQ ID NO: 186.

Provided herein is a bispecific antibody, comprising:
(a) a first heavy chain polypeptide comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and (b) a second heavy chain polypeptide comprising from N- to C-terminal order: a V_{H} region comprising the sequence set forth in SEQ ID NO: 72, a linker of SEQ ID NO: 162, a VL region comprising the sequence set forth in SEQ ID NO: 73, and a Fc region comprising the sequence set forth in SEQ ID NO:165. In some embodiments, the first heavy chain polypeptide comprises from N to C-terminal order the scFv comprising the sequence set forth in SEQ ID NO:248 and the Fc region comprising the sequence set forth in SEQ ID NO:166; and the second heavy chain polypeptide comprises from N to C-terminal order the scFv comprising the sequence set forth in SEQ ID NO:247 and the Fc region comprising the sequence set forth in SEQ ID NO: 165.

In some of any embodiments, the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 187; and (b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 188. In some of any embodiments, (a) the first heavy chain polypeptide comprises the amino acid sequence of SEQ ID NO: 187; and (b) the second heavy chain polypeptide comprises the amino acid sequence of SEQ ID NO: 188.

Provided herein is a bispecific antibody, comprising:
(a) a first heavy chain polypeptide comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and (b) a second heavy chain polypeptide comprising from N- to C-terminal order: a Fab comprising a VH region comprising the sequence set forth in SEQ ID NO: 70 and an IgG1 CH1 of SEQ ID NO: 169, and a Fc region comprising the sequence set forth in SEQ ID NO:165; and (c) a light chain comprises a VL region comprising the sequence set forth in SEQ ID NO: 43 and a CL comprising the sequence set forth in SEQ ID NO: 171. In some embodiments, the first heavy chain polypeptide comprises from N to C-terminal order the scFv comprising the sequence set forth in SEQ ID NO:248 and the Fc region comprising the sequence set forth in SEQ ID NO:166.

In some of any embodiments, the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 189;
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 190; and
(c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 191.

In some of any embodiments, (a) the first heavy chain polypeptide comprises the amino acid sequence of SEQ ID NO: 189; (b) the second heavy chain polypeptide comprises the amino acid sequence of SEQ ID NO: 190; and (c) the light chain comprises the amino acid sequence of SEQ ID NO: 191.

Provided herein is a bispecific antibody, comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 168; and
(b) a second heavy chain polypeptide comprising from N- to C-terminal order: a Fab comprising a V_{H} region comprising the sequence set forth in SEQ ID NO: 68 and an IgG1 CH1 of SEQ ID NO: 170, and a Fc region comprising the sequence set forth in SEQ ID NO:167; and
(c) a light chain comprises a VL region comprising the sequence set forth in SEQ ID NO: 39 and a CL comprising the sequence set forth in SEQ ID NO: 171.

In some embodiments, the first heavy chain polypeptide comprises from N- to C-terminal order the scFv comprising the sequence set forth in SEQ ID NO: 245 and the Fc region comprising the sequence set forth in SEQ ID NO:168. In some of any embodiments, the bispecific antibody comprises:(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 234; (b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 235; and (c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 236. In some of any embodiments, (a) the first heavy chain polypeptide comprises the amino acid sequence of SEQ ID NO: 234; (b) the second heavy chain polypeptide comprises the amino acid sequence of SEQ ID NO: 235; and (c) the light chain comprises the amino acid sequence of SEQ ID NO: 236.

Provided herein is a bispecific antibody, comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 168; and
(b) a second heavy chain polypeptide comprising from N- to C-terminal order: a Fab comprising a V_{H} region comprising the sequence set forth in SEQ ID NO: 70 and an IgG1 CH1 of SEQ ID NO: 170, and a Fc region comprising the sequence set forth in SEQ ID NO: 167; and
(c) a light chain comprises a VL region comprising the sequence set forth in SEQ ID NO: 43 and a CL comprising the sequence set forth in SEQ ID NO: 171.

In some embodiments, the first heavy chain polypeptide comprises from N- to C-terminal order the scFv comprising the sequence set forth in SEQ ID NO: 245 and the Fc region comprising the sequence set forth in SEQ ID NO:168. In some of any embodiments, the bispecific antibody comprises: (a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 237; (b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 238; and (c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 239. In some of any embodiments, (a) the first heavy chain polypeptide comprises the amino acid sequence of SEQ ID NO: 237; (b) the second heavy chain polypeptide comprises the amino acid sequence of SEQ ID NO: 238; and (c) the light chain comprises the amino acid sequence of SEQ ID NO: 239.

Provided herein is a polynucleotide comprising a nucleic acid sequence(s) encoding the antibody or antigen-binding fragment disclosed herein or the bispecific antibody disclosed herein. Provided herein is a polynucleotide system comprising a nucleic acid sequence encoding each chain of the antibody or antigen-binding fragment disclosed herein or the bispecific antibody disclosed herein. Provided herein is a polynucleotide system comprising (a) a first nucleic acid sequence encoding the first heavy chain of the bispecific antibody disclosed herein, and (b) a second nucleic acid sequence encoding the second heavy chain of the bispecific antibody disclosed herein.

An aspect of the invention provides a polynucleotide comprising a nucleic acid comprising a nucleic acid sequence encoding any of the binding molecules or antigen binding fragments thereof described herein. In certain embodiments, the nucleic acid sequences encodes any of the binding molecules or antigen binding fragments thereof described herein including those that contain binding domains or antibodies listed in Table E4, Table E5, Table E6, Table E8, and/or Table E9.

Provided herein is a vector comprising the polynucleotide disclosed herein. An aspect of the invention provides a vector useful for expressing a binding molecule described herein comprising the polynucleotide. In certain embodiments, the vector is used to express any of the binding molecules or antigen binding fragments thereof described herein including those that comprise a binding domain sequence, or a bispecific antibody sequence, listed in Table E4, Table E5, Table E6, Table E8, and/or Table E9. In some embodiments, the vector is an expression vector. In certain embodiments, the vector is a viral vector. For example, the viral vector is a retroviral vector or a lentiviral vector. Provided herein is a vector system comprising a plurality of vectors, each vector comprising a nucleic acid sequence of the polynucleotide system disclosed herein. In some embodiments, each vector is an expression vector.

Provided herein is a cell comprising the polynucleotide of disclosed herein or the vector disclosed herein. Provided herein is a cell comprising the polynucleotide system disclosed herein, or the vector system disclosed herein. In some of any embodiments, the cell is a mammalian cell. An aspect of the invention provides a cell useful for expressing any of the binding molecules or antigen binding fragments thereof described herein comprising the polynucleotide, or the vector. In certain embodiments, the cell is used to express any of the binding molecules or antigen binding fragments thereof described herein including those that comprise a binding domain sequence, or a bispecific antibody sequence, listed in Table E4, Table E5, Table E6, Table E8, and/or Table E9. In certain embodiments, the cell is a mammalian cell. In certain embodiments, the cell is a CHO cell. In certain embodiments, the cell is a NS0 cell. In certain embodiments, the cell is a SP2/0 cell. In certain embodiments, the cell is a HEK293 cell. In certain embodiments, the cell is a PER.C6 cell. In certain embodiments, the cell is a BHK-21 cell.

Provided herein is a method of producing an antibody comprising culturing the cell disclosed herein under a condition that produces the antibody. An aspect of the invention provides a method of producing any of the binding molecules or antigen binding fragments thereof described herein comprising culturing the cell under a condition that produces the binding molecule. In certain embodiments, the binding molecule is an antibody. In certain embodiments, the binding molecule or antigen binding fragment thereof is described herein including those that comprise a binding domain sequence, or a bispecific antibody sequence, listed in Table E4, Table E5, Table E6, Table E8, and/or Table E9. In certain embodiments, the method further comprises recovering the antibody produced by the cell.

Provided herein is an antibody or antigen-binding fragment thereof or bispecific antibody produced by the method disclosed herein. An aspect of the invention provides any binding molecule (e.g., antibody) or antigen-binding fragment thereof produced by the method. In certain embodiments, the binding molecule or antigen binding fragment thereof is described herein including those that comprise a binding domain sequence, or a bispecific antibody sequence, listed in Table E4, Table E5, Table E6, Table E8, and/or Table E9.

Provided herein is a composition comprising the antibody or antigen-binding fragment disclosed herein. Provided herein is a composition comprising the bispecific antibody disclosed herein. An aspect of the invention provides a composition comprising any of the binding molecules or antigen binding fragment thereof described herein. In certain embodiments, the binding molecule or antigen binding fragment thereof is described herein including those that comprise a binding domain sequence, or a bispecific antibody sequence, listed in Table E4, Table E5, Table E6, Table E8, and/or Table E9. In certain embodiments, the composition further comprises a pharmaceutically acceptable excipient.

Provided herein is a method for treating a disease or condition in a subject in need thereof, the method comprising administering a therapeutically effective amount of the antibody or antigen-binding fragment disclosed herein or composition disclosed herein. Provided herein is a method for treating a disease or condition in a subject in need thereof, the method comprising administering a therapeutically effective amount of the bispecific antibody disclosed herein or composition disclosed herein. An aspect of the invention provides a method for treating a subject with a disease or condition, the method comprising administering a therapeutically effective amount of any of the binding molecules or antigen binding fragments thereof described herein. In certain embodiments, the binding molecule or antigen binding fragment thereof is described herein including those that comprise a binding domain sequence, or a bispecific antibody sequence, listed in Table E4, Table E5, Table E6, Table E8, and/or Table E9.

In certain embodiments of the method, the disease or condition is an infectious disease or disorder, an autoimmune disease, an inflammatory disease, or a tumor or a cancer. In certain embodiments of the method, the disease or condition is associated with overexpression of B cells in tissues.

In certain embodiments of the method, the disease or condition is an autoimmune disease. In certain embodiments, the autoimmune disease in the subject is associated with presence of increased autoantibodies and/or autoreactive B cells in the subject compared to a healthy subject.

In some of any embodiments, the autoimmune disorder is selected from the group consisting of psoriasis, psoriatic arthritis, Type 1 diabetes mellitus (Type 1 diabetes), rheumatoid arthritis (RA), systemic lupus erythematosus (SLE), cutaneous lupus erythematosus (CLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, erythematosus (DLE), dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

In certain embodiments of the method, the disease or condition is systemic lupus erythematosus (SLE), rheumatoid arthritis, Sjögren disease, or systemic sclerosis. In some of any embodiments, the autoimmune disorder is SLE.

In some of any embodiments, the disease or condition is a cancer. In some of any embodiments, the cancer is a solid tumor. In some of any embodiments, the cancer is a melanoma, breast carcinoma, HCC, Renal cell carcinoma, Colorectal carcinoma, non-small-cell lung carcinoma, Cervical cancer or Mesothelioma.

In some of any embodiments, the cancer is a B-cell-related cancer selected from the group consisting of multiple myeloma, malignant plasma cell neoplasm, plasma cell leukemia, plasmacytoma, B-cell prolymphocytic leukemia, hairy cell leukemia, B-cell non-Hodgkin's lymphoma (NHL), chronic lymphocytic leukemia (CLL), follicular lymphoma, Burkitt's lymphoma, marginal zone lymphoma, mantle cell lymphoma, large cell lymphoma, precursor B-lymphoblastic lymphoma, Waldenstrom's macroglobulinemia, diffuse large B-cell lymphoma (DLBCL), mucosa-associated lymphatic tissue lymphoma (MALT lymphoma), small cell lymphocytic lymphoma, Primary mediastinal (thymic) large B-cell lymphoma, lymphoplasmacytic lymphoma, nodal marginal zone B cell lymphoma, splenic marginal zone lymphoma, intravascular large B-cell lymphoma, primary effusion lymphoma, lymphomatoid granulomatosis, T cell/histiocyte-rich large B-cell lymphoma, primary CNS (central nervous system) lymphoma, primary cutaneous diffuse large B-cell lymphoma (leg type), EBV positive diffuse large B-cell lymphoma of the elderly, diffuse large B-cell lymphoma associated with inflammation, ALK-positive large B-cell lymphoma, and plasmablastic lymphoma. In some of any embodiments, the disease or condition is an inflammatory disease. In some of any embodiments, the inflammatory disease is selected from the group consisting of asthma, atopic dermatitis (eczema), contact dermatitis and gout.

Provided herein is a kit comprising the antibody or antigen-binding fragment disclosed herein, or the bispecific antibody disclosed herein and, optionally, instructions for use. An aspect of the invention provides a kit comprising any of the binding molecule or antigen binding fragments thereof described herein. In certain embodiments, the binding molecule or antigen binding fragment thereof is described herein including those that comprise a binding domain sequence, or a bispecific antibody sequence, listed in Table E4, Table E5, Table E6, Table E8, and/or Table E9. In certain embodiments, the kit further comprises instructions for use. In certain embodiments, the instructions for use comprises instructions for performing any of the methods described herein.

### BRIEF DESCRIPTION OF THE DRAWINGS

FIG. 1A and FIG. 1B depict bispecific antibodies in the scFv-Fc×Fab-Fc format (FIG. 1A; i.e., one arm presenting a single-chain variable fragment (scFv) fused to Fc, the other arm presenting a Fab fragment fused to Fc) and the scFv-Fc×scFv-Fc format (FIG. 1B; i.e., each arm presenting a single-chain variable fragment (scFv) fused to Fc).

FIG. 2A and FIG. 2B depict two formats of CD19 TCEs that were generated using binders 13-CD19 with 6-CD3, and 13-CD19 with 5-CD3, respectively.

FIG. 3A and FIG. 3B depict the ability of TCE constructs with different CD19 binders and in two different formats (i.e., the scFv-Fc×scFv-Fc TCE format (FIG. 3A); the scFv-Fc×Fab-Fc TCE format (FIG. 3B)) to deplete Raji B cells.

FIGS. 4A-4D are graphs showing the ability of a selection of TCEs to deplete human B cells (FIG. 4A) and to induce with a lower potency secretion of cytokines including IFNγ (FIG. 4B), TNFα (FIG. 4C) and IL-6 (FIG. 4D) in a human PBMC assay.

FIGS. 5A-5D are graphs showing the ability of a selection of TCEs to deplete B cells (FIG. 5A) and to induce secretion of cytokines including, IFNγ (FIG. 5B), TNFα (FIG. 5C) and IL-6 (FIG. 5D) in a cynomolgus macaques (cyno) PBMC assay.

FIGS. 6A-6C are graphs showing in vivo B cell depletion in whole blood (FIG. 6A), spleen (FIG. 6B), and bone marrow (FIG. 6C) of hCD19×hCD3-epitope transgenic mice at 72 hours post subcutaneous administration of selected TCEs including TCE001, TCE009, and TCE003. The mean % B cell depletion is shown at the top of each corresponding bar graph.

FIG. 7A and FIG. 7B are graphs showing IFNγ and IL-6 and cytokine levels, respectively, in the serum of hCD19×hCD3ε transgenic mice 6 hours post-subcutaneous administration of selected TCE molecules, including TCE001, TCE009, and TCE003.

FIGS. 8A-8B depict a comparative analysis of the B cell depletion efficiency versus IL-6 secretion levels between TCE001 (FIG. 8A) and blinatumomab bs (FIG. 8B).

FIGS. 9A-9B depict a comparative analysis of the B cell depletion efficiency versus TNFα secretion levels between TCE001 (FIG. 9A) and blinatumomab bs (FIG. 9B).

FIGS. 10A-10C depict the results of an in vitro PBMC depletion assay and a cytokine release assay (i.e., IFNγ at B cell depletion EC₅₀ (FIG. 10A), TNFα at B cell depletion EC₅₀ (FIG. 10B), and IL-6 at B cell depletion EC₅₀ (FIG. 10C)) performed using TCE001, blinatumomab bs and tAb0032.

### DETAILED DESCRIPTION

The present invention provides a binding molecule or antibody construct comprising a first binding domain which binds to CD19, and a second binding domain which binds to CD3. In certain embodiments, the binding molecule or antibody construct further comprises two polypeptide monomers, each comprising a hinge, a CH2 domain and a CH3 domain, wherein said two polypeptide monomers are fused to each other via a peptide linker. In some embodiments, the binding molecule is a bispecific antibody. In some embodiments, provided herein are antibodies that bind to human CD3 and human CD19. The binding molecules, such as bispecific antibodies, described herein are designed to engage and deplete B cells by redirecting T cell cytotoxic activity toward B cells expressing CD19. In some embodiments, the B cells may by any B cell lineage, including immature, mature, and pathogenic B cells. By promoting targeted B cell depletion, these binding molecules can be used to reset the B cell compartment and thereby treat or ameliorate autoimmune diseases and other B cell-associated disorders.

In certain embodiments, the binding molecules described herein deplete B cells by redirecting T cell cytotoxic activity toward CD19-expressing B lineage cells. B cells play a central role in diseases such as autoimmune disease and cancer. For example, B cells may be involved in autoimmune disease through antibody production, antigen presentation, and cytokine secretion. Dysregulated B cell activation and persistence of autoreactive plasma cells contribute to disease chronicity and relapse. By engaging CD3 on T cells, the binding molecules recruit and activate cytotoxic T lymphocytes in proximity to pathogenic B cells, resulting in targeted elimination of B cells and plasma cells. This mechanism allows for efficient B cell depletion and provides a means to reset the B cell compartment in autoimmune disease.

In some embodiments, the binding molecules or antibody constructs described herein are administered to a subject having a severe or moderate systemic autoimmune disease, e.g., via adoptive cell therapy, such as adoptive T cell therapy. In some aspects, the disease or condition is a systemic autoimmune disease. In some aspects, the disease or condition is severe or moderate systemic autoimmune disease. Systemic autoimmune diseases are a class of aberrant immune disorders that share similar clinical manifestations and generally are treatable by similar approaches. In addition to Systemic Lupus Erythematosus (SLE), other systemic autoimmune diseases include, for example, Sjogren's′ syndrome, progressive systemic sclerosis (i.e., scleroderma), idiopathic inflammatory myositis (IIM, including dermatomyositis, polymyositis and necrotizing myositis), mixed connective tissue disorder (MCTD), relapsing-remitting multiple sclerosis, ANCA-associated vasculitis (AAV), Crohn's disease, myasthenia gravis, Behçet's disease, rheumatoid arthritis, multiple sclerosis (MS), IgA nephropathy, pemphigus vulgaris, myasthenia gravis, autoimmune hemolytic anemia, immune thrombocytopenia, IgG4-related diseases, membranous nephropathy, cutaneous lupus erythematosus, sarcoidosis, light chain amyloidosis, rheumatoid arthritis, bullous pemphigoid and chronic inflammatory demyelinating polyneuropathy.

In some embodiments, the binding molecules or antibody constructs described herein, are used in therapy wherein binding molecules or antibody constructs described herein bind to CD19 expressed by cells (e.g., B cells) that play a role in the manifestation of the systemic autoimmune disease. In some embodiments, CD19 is expressed by cells, associated with and/or specific to the manifestation of SLE, IIM, SSc, AAV, systemic sclerosis, highly active relapsing remitting multiple sclerosis (MS), primary progressive MS, IgA nephropathy, pemphigus vulgaris, myasthenia gravis, demyelinating polyradiculoneuropathy, autoimmune hemolytic anemia, immune thrombocytopenia, IgG4-related diseases, membranous nephropathy, Primary Sjorgren's Syndrome, cutaneous lupus erythematosus, sarcoidosis, light chain amyloidosis, rheumatoid arthritis, bullous pemphigoid, acute respiratory distress syndrome, atopic eczema, hereditary angioedema, hidradenitis suppurative, inclusion-body myositis, inflammatory bowel disease, mastocytosis, multifocal motor neuropathy, necrotizing myopathy, neuromyelitis optica spectrum disorder, mixed connective tissue disorder, POEMS syndrome, primary biliary cholangitis, psoriasis, rhesus hemolytic disease, Still's disease, type 1 diabetes, urticaria, capillary leakage syndrome, cytokine release syndrome, erythema multiforme, pyoderma gangrenosum, antiphospholipid syndrome, or x-linked agammaglobulinemia. In some embodiments, CD19 is expressed by cells, associated with and/or specific to the manifestation of SLE, IIM, AAV, systemic sclerosis, highly active relapsing remitting multiple sclerosis (MS), primary progressive MS, IgA nephropathy, pemphigus vulgaris, or myasthenia gravis.

In particular embodiments, CD19 is expressed by cells, associated with and/or specific to the manifestation of SLE, such as severe refractory SLE. In particular embodiments, CD19 is expressed by cells, associated with and/or specific to the manifestation of idiopathic inflammatory myopathies (IIM). In particular embodiments, CD19 is expressed by cells, associated with and/or specific to the manifestation of systemic sclerosis (SSc). In particular embodiments, CD19 is expressed by cells, associated with and/or specific to the manifestation of multiple sclerosis (MS). In particular embodiments, CD19 is expressed by cells, associated with and/or specific to the manifestation of rheumatoid arthritis (RA). In particular embodiments, CD19 is expressed by cells, associated with and/or specific to the manifestation of active secondary progressive MS (aSPMS). In particular embodiments, CD19 is expressed by cells, associated with and/or specific to the manifestation of Myositis. In particular embodiments, CD19 is expressed by cells, associated with and/or specific to the manifestation of myasthenia gravis. In particular embodiments, CD19 is expressed by cells, associated with and/or specific to the manifestation of bullous pemphigoid. In particular embodiments, CD19 is expressed by cells, associated with and/or specific to the manifestation of immune thrombocytopenia. In particular embodiments, CD19 is expressed by cells, associated with and/or specific to the manifestation of autoimmune hemolytic anemia. In particular embodiments, CD19 is expressed by cells, associated with and/or specific to the manifestation of pemphigus vulgaris. In particular embodiments, CD19 is expressed by cells, associated with and/or specific to the manifestation of demyelinating polyradiculoneuropathy. In particular embodiments, CD19 is expressed by cells, associated with and/or specific to the manifestation of membranous nephropathy.

In some embodiments, the systemic autoimmune disease is SLE, IIM, MS, or SSc. In some aspects, the disease or condition is moderate SLE. In some aspects, the disease or condition is severe refractory SLE. In particular, provided herein are binding molecules or antibody constructs and/or compositions thereof, for the treatment of subjects having severe refractory SLE. In embodiments of the provided methods, the binding molecules or antibody constructs are administered to a subject having severe refractory SLE.

In immunology, affinity maturation is the process by which B cells produce antibodies with increased affinity for antigen during the course of an immune response. With repeated exposures to the same antigen, a host will produce antibodies of successively greater affinities. Like the natural prototype, the in vitro affinity maturation is based on the principles of mutation and selection. The in vitro affinity maturation has successfully been used to optimize antibodies, antibody constructs, and antibody fragments. Random mutations inside the CDRs are introduced using radiation, chemical mutagens or error-prone PCR. In addition, the genetic diversity can be increased by chain shuffling. Two or three rounds of mutation and selection using display methods like phage display usually results in antibody fragments with affinities in the low nanomolar range.

A preferred type of an amino acid substitutional variation of the antibody constructs involves substituting one or more hypervariable region residues of a parent antibody (e.g. a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sides (e.g., 6-7 sides) are mutated to generate all possible amino acid substitutions at each side. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sides for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the binding domain and the antigen, e.g., human CD19 and human CD3. Such contact residues and neighboring residues are candidates for substitution according to the techniques elaborated herein. Once such variants are generated, the panel of variants is subjected to screening as described herein and antibodies with superior properties in one or more relevant assays may be selected for further development.

The monoclonal antibodies and antibody constructs of the present invention specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is/are identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81: 6851-6855 (1984)). Chimeric antibodies of interest herein include “primitized” antibodies comprising variable domain antigen-binding sequences derived from a non-human primate (e.g., Old World Monkey, Ape etc.) and human constant region sequences. A variety of approaches for making chimeric antibodies have been described. See e.g., Morrison et al., Proc. Natl. Acad. ScL U.S.A. 81:6851, 1985; Takeda et al., Nature 314:452, 1985, Cabilly et al., U.S. Pat. No. 4,816,567; Boss et al., U.S. Pat. No. 4,816,397; Tanaguchi et al., EP 0171496; EP 0173494; and GB 2177096.

An antibody, antibody construct, antibody fragment or antibody variant may also be modified by specific deletion of human T cell epitopes (a method called “deimmunization”) by the methods disclosed for example in WO 98/52976 or WO 00/34317. Briefly, the heavy and light chain variable domains of an antibody can be analyzed for peptides that bind to MHC class II; these peptides represent potential T cell epitopes (as defined in WO 98/52976 and WO 00/34317). For detection of potential T cell epitopes, a computer modeling approach termed “peptide threading” can be applied, and in addition a database of human MHC class II binding peptides can be searched for motifs present in the VH and VL sequences, as described in WO 98/52976 and WO 00/34317. These motifs bind to any of the 18 major MHC class II DR allotypes, and thus constitute potential T cell epitopes. Potential T cell epitopes detected can be eliminated by substituting small numbers of amino acid residues in the variable domains, or preferably, by single amino acid substitutions. Typically, conservative substitutions are made. Often, but not exclusively, an amino acid common to a position in human germline antibody sequences may be used. Human germline sequences are disclosed e.g. in Tomlinson, et al. (1992) J. Mol. Biol. 227:776-798; Cook, G. P. et al. (1995) Immunol. Today Vol. 16 (5): 237-242; and Tomlinson et al. (1995) EMBO J. 14: 14:4628-4638. The V BASE directory provides a comprehensive directory of human immunoglobulin variable region sequences (compiled by Tomlinson, L A. et al. MRC Centre for Protein Engineering, Cambridge, UK). These sequences can be used as a source of human sequence, e.g., for framework regions and CDRs. Consensus human framework regions can also be used, for example as described in U.S. Pat. No. 6,300,064.

“Humanized” antibodies, antibody constructs, variants or fragments thereof (such as Fv, Fab, Fab′, F(ab′)2 or other antigen-binding subsequences of antibodies) are antibodies or immunoglobulins of mostly human sequences, which contain (a) minimal sequence(s) derived from non-human immunoglobulin. For the most part, humanized antibodies are human immunoglobulins (recipient antibody) in which residues from a hypervariable region (also CDR) of the recipient are replaced by residues from a hypervariable region of a non-human (e.g., rodent) species (donor antibody) such as mouse, rat, hamster or rabbit having the desired specificity, affinity, and capacity. In some instances, Fv framework region (FR) residues of the human immunoglobulin are replaced by corresponding non-human residues. Furthermore, “humanized antibodies” as used herein may also comprise residues which are found neither in the recipient antibody nor the donor antibody. These modifications are made to further refine and optimize antibody performance. The humanized antibody may also comprise at least a portion of an immunoglobulin constant region (Fc), typically that of a human immunoglobulin. For further details, see Jones et al., Nature, 321: 522-525 (1986); Reichmann et al., Nature, 332: 323-329 (1988); and Presta, Curr. Op. Struct. Biol., 2: 593-596 (1992).

Humanized antibodies or fragments thereof can be generated by replacing sequences of the Fv variable domain that are not directly involved in antigen binding with equivalent sequences from human Fv variable domains. Exemplary methods for generating humanized antibodies or fragments thereof are provided by Morrison (1985) Science 229:1202-1207; by Oi et al. (1986) BioTechniques 4:214; and by U.S. Pat. Nos. 5,585,089; 5,693,761; 5,693,762; 5,859,205; and 6,407,213. Those methods include isolating, manipulating, and expressing the nucleic acid sequences that encode all or part of immunoglobulin Fv variable domains from at least one of a heavy or light chain. Such nucleic acids may be obtained from a hybridoma producing an antibody against a predetermined target, as described above, as well as from other sources. The recombinant DNA encoding the humanized antibody molecule can then be cloned into an appropriate expression vector.

Humanized antibodies may also be produced using transgenic animals such as mice that express human heavy and light chain genes, but are incapable of expressing the endogenous mouse immunoglobulin heavy and light chain genes. Winter describes an exemplary CDR grafting method that may be used to prepare the humanized antibodies described herein (U.S. Pat. No. 5,225,539). All of the CDRs of a particular human antibody may be replaced with at least a portion of a non-human CDR, or only some of the CDRs may be replaced with non-human CDRs. It is only necessary to replace the number of CDRs required for binding of the humanized antibody to a predetermined antigen.

A humanized antibody can be optimized by the introduction of conservative substitutions, consensus sequence substitutions, germline substitutions and/or back mutations. Such altered immunoglobulin molecules can be made by any of several techniques known in the art, (e.g., Teng et al., Proc. Natl. Acad. Sci. U.S.A., 80: 7308-7312, 1983; Kozbor et al., Immunology Today, 4: 7279, 1983; Olsson et al., Meth. Enzymol., 92: 3-16, 1982, and EP 239 400).

According to the present invention, binding domains are in the form of one or more polypeptides. Such polypeptides may include proteinaceous parts and non-proteinaceous parts (e.g. chemical linkers or chemical cross-linking agents such as glutaraldehyde). Proteins (including fragments thereof, preferably biologically active fragments, and peptides, usually having less than 30 amino acids) comprise two or more amino acids coupled to each other via a covalent peptide bond (resulting in a chain of amino acids).

Preferably the binding domain which binds to CD19 and/or the binding domain which binds to CD3 is/are human binding domains. Antibodies and antibody constructs comprising at least one human binding domain avoid some of the problems associated with antibodies or antibody constructs that possess non-human such as rodent (e.g. murine, rat, hamster or rabbit) variable and/or constant regions. The presence of such rodent derived proteins can lead to the rapid clearance of the antibodies or antibody constructs or can lead to the generation of an immune response against the antibody or antibody construct by a patient. In order to avoid the use of rodent derived antibodies or antibody constructs, human or fully human antibodies/antibody constructs can be generated through the introduction of human antibody function into a rodent so that the rodent produces fully human antibodies.

The ability to clone and reconstruct megabase-sized human loci in YACs and to introduce them into the mouse germline provides a powerful approach to elucidating the functional components of very large or crudely mapped loci as well as generating useful models of human disease. Furthermore, the use of such technology for substitution of mouse loci with their human equivalents could provide unique insights into the expression and regulation of human gene products during development, their communication with other systems, and their involvement in disease induction and progression.

An important practical application of such a strategy is the “humanization” of the mouse humoral immune system. Introduction of human immunoglobulin (Ig) loci into mice in which the endogenous Ig genes have been inactivated offers the opportunity to study the mechanisms underlying programmed expression and assembly of antibodies as well as their role in B-cell development. Furthermore, such a strategy could provide an ideal source for production of fully human monoclonal antibodies (mAbs)—an important milestone towards fulfilling the promise of antibody therapy in human disease. Fully human antibodies or antibody constructs are expected to minimize the immunogenic and allergic responses intrinsic to mouse or mouse-derivatized mAbs and thus to increase the efficacy and safety of the administered antibodies/antibody constructs. The use of fully human antibodies or antibody constructs can be expected to provide a substantial advantage in the treatment of chronic and recurring human diseases, such as inflammation, autoimmunity, and B cell disorders, which require repeated compound administrations.

One approach towards this goal was to engineer mouse strains deficient in mouse antibody production with large fragments of the human Ig loci in anticipation that such mice would produce a large repertoire of human antibodies in the absence of mouse antibodies. Large human Ig fragments would preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains should yield high affinity antibodies against any antigen of interest, including human antigens. Using the hybridoma technology, antigen-specific human mAbs with the desired specificity could be readily produced and selected. This general strategy was demonstrated in connection with the generation of the first XenoMouse mouse strains (see Green et al. Nature Genetics 7:13-21 (1994)). The XenoMouse strains were engineered with yeast artificial chromosomes (YACs) containing 245 kb and 190 kb-sized germline configuration fragments of the human heavy chain locus and kappa light chain locus, respectively, which contained core variable and constant region sequences. The human Ig containing YACs proved to be compatible with the mouse system for both rearrangement and expression of antibodies and were capable of substituting for the inactivated mouse Ig genes. This was demonstrated by their ability to induce B cell development, to produce an adult-like human repertoire of fully human antibodies, and to generate antigen-specific human mAbs. These results also suggested that introduction of larger portions of the human Ig loci containing greater numbers of V genes, additional regulatory elements, and human Ig constant regions may recapitulate substantially the full repertoire that is characteristic of the human humoral response to infection and immunization. The work of Green et al. was recently extended to the introduction of greater than approximately 80% of the human antibody repertoire through introduction of megabase sized, germline configuration YAC fragments of the human heavy chain loci and kappa light chain loci, respectively. See Mendez et al. Nature Genetics 15:146-156 (1997) and U.S. patent application Ser. No. 08/759,620.

The production of the XenoMouse mice is further discussed and delineated in U.S. patent application Ser. No. 07/466,008, Ser. No. 07/610,515, Ser. No. 07/919,297, Ser. No. 07/922,649, Ser. No. 08/031,801, Ser. No. 08/112,848, Ser. No. 08/234,145, Ser. No. 08/376,279, Ser. No. 08/430,938, Ser. No. 08/464,584, Ser. No. 08/464,582, Ser. No. 08/463,191, Ser. No. 08/462,837, Ser. No. 08/486,853, Ser. No. 08/486,857, Ser. No. 08/486,859, Ser. No. 08/462,513, Ser. No. 08/724,752, and Ser. No. 08/759,620; and U.S. Pat. Nos. 6,162,963; 6,150,584; 6,114,598; 6,075,181, and 5,939,598 and Japanese Patent Nos. 3,068,180 B2, 3,068,506 B2, and 3,068,507 B2. See also Mendez et al. Nature Genetics 15:146-156 (1997) and Green and Jakobovits J. Exp. Med. 188:483-495 (1998), EP 0 463 151 B1, WO 94/02602, WO 96/34096, WO 98/24893, WO 00/76310, and WO 03/47336.

In an alternative approach, others, including GenPharm International, Inc., have utilized a “minilocus” approach. In the minilocus approach, an exogenous Ig locus is mimicked through the inclusion of pieces (individual genes) from the Ig locus. Thus, one or more VH genes, one or more DH genes, one or more JH genes, a mu constant region, and a second constant region (preferably a gamma constant region) are formed into a construct for insertion into an animal. This approach is described in U.S. Pat. No. 5,545,807 to Surani et al. and U.S. Pat. Nos. 5,545,806; 5,625,825; 5,625,126; 5,633,425; 5,661,016; 5,770,429; 5,789,650; 5,814,318; 5,877,397; 5,874,299; and 6,255,458 each to Lonberg and Kay, U.S. Pat. Nos. 5,591,669 and 6,023,010 to Krimpenfort and Berns, U.S. Pat. Nos. 5,612,205; 5,721,367; and U.S. Pat. No. 5,789,215 to Berns et al., and U.S. Pat. No. 5,643,763 to Choi and Dunn, and GenPharm International U.S. patent application Ser. No. 07/574,748, Ser. No. 07/575,962, Ser. No. 07/810,279, Ser. No. 07/853,408, Ser. No. 07/904,068, Ser. No. 07/990,860, Ser. No. 08/053,131, Ser. No. 08/096,762, Ser. No. 08/155,301, Ser. No. 08/161,739, Ser. No. 08/165,699, Ser. No. 08/209,741. See also EP 0 546 073 B1, WO 92/03918, WO 92/22645, WO 92/22647, WO 92/22670, WO 93/12227, WO 94/00569, WO 94/25585, WO 96/14436, WO 97/13852, and WO 98/24884 and U.S. Pat. No. 5,981,175. See further Taylor et al. (1992), Chen et al. (1993), Tuaillon et al. (1993), Choi et al. (1993), Lonberg et al. (1994), Taylor et al. (1994), and Tuaillon et al. (1995), Fishwild et al. (1996).

Kirin has also demonstrated the generation of human antibodies from mice in which, through microcell fusion, large pieces of chromosomes, or entire chromosomes, have been introduced. See European Patent Application Nos. 773 288 and 843 961. Xenerex Biosciences is developing a technology for the potential generation of human antibodies. In this technology, SCID mice are reconstituted with human lymphatic cells, e.g., B and/or T cells. Mice are then immunized with an antigen and can generate an immune response against the antigen. See U.S. Pat. Nos. 5,476,996; 5,698,767; and 5,958,765.

Human anti-mouse antibody (HAMA) responses have led the industry to prepare chimeric or otherwise humanized antibodies. It is however expected that certain human anti-chimeric antibody (HACA) responses will be observed, particularly in chronic or multi-dose utilizations of the antibody. Thus, it would be desirable to provide antibody constructs comprising a human binding domain against CD19 and a human binding domain against CD3 in order to vitiate concerns and/or effects of HAMA or HACA response.

By way of example, for the treatment of B cell disorders, a therapeutically effective amount or dosage of the drug or therapeutic agent (e.g., an anti-CD19 antibody or antigen binding portion thereof, or a CD19/CD3 binding molecule or antibody construct) reduces B cells by at least about 20%, by at least about 30% by at least about 40%, by at least about 50%, by at least about 60%, by at least above 70%, by at least about 80%, or by at least about 90% relative to untreated subjects. In some aspects, a therapeutically effective amount or dosage of the drug or therapeutic agent completely depletes B cells, i.e., inhibits B cell populations or B cell growth by 100%. The ability of a drug or therapeutic agent, including an antibody, to deplete B cells can be evaluated using the assays described herein. Alternatively, this property of a composition comprising the compound or therapeutic agent can be evaluated by examining the ability of the composition to inhibit B growth; such inhibition can be measured in vitro by assays known to the skilled practitioner.

A method for epitope mapping is described in the following: When a region (a contiguous amino acid stretch) in the human CD19 protein is exchanged/replaced with its corresponding region of a non-human and non-primate CD19 (e.g., mouse CD19, but others like chicken, rat, hamster, rabbit etc. may also be conceivable), a decrease in the binding of the binding domain is expected to occur, unless the binding domain is cross-reactive for the non-human, non-primate CD19 used. Said decrease is preferably at least 10%, 20%, 30%, 40%, or 50%; more preferably at least 60%, 70%, or 80%, and most preferably 90%, 95% or even 100% in comparison to the binding to the respective region in the human CD19 protein, whereby binding to the respective region in the human CD19 protein is set to be 100%. It is envisaged that the aforementioned human CD19/non-human CD19 chimeras are expressed in CHO cells.

In an alternative or additional method for epitope mapping, several truncated versions of the human CD19 extracellular domain can be generated in order to determine a specific region that is recognized by a binding domain. In these truncated versions, the different extracellular CD19 domains/sub-domains or regions are stepwise deleted, starting from the N-terminus. It is envisaged that the truncated CD19 versions may be expressed in CHO cells. It is also envisaged that the truncated CD19 versions may be fused with a transmembrane domain and/or cytoplasmic domain of a different membrane-bound protein. It is also envisaged that the truncated CD19 versions may encompass a signal peptide domain at their N-terminus, for example a signal peptide derived from mouse IgG heavy chain signal peptide. It is furthermore envisaged that the truncated CD19 versions may encompass a v5 domain at their N-terminus (following the signal peptide) which allows verifying their correct expression on the cell surface. A decrease or a loss of binding is expected to occur with those truncated CD19 versions which do not encompass any more the CD19 region that is recognized by the binding domain. The decrease of binding is preferably at least 10%, 20%, 30%, 40%, 50%; more preferably at least 60%, 70%, 80%, and most preferably 90%, 95% or even 100%, whereby binding to the entire human CD19 protein (or its extracellular region or domain) is set to be 100.

A further method to determine the contribution of a specific residue of CD19 to the recognition by an antibody construct or binding domain is alanine scanning (see e.g. Morrison K L & Weiss G A. Cur Opin Chem Biol. 2001 June; 5(3):302-7), where each residue to be analyzed is replaced by alanine, e.g. via site-directed mutagenesis. Alanine is used because of its non-bulky, chemically inert, methyl functional group that nevertheless mimics the secondary structure references that many of the other amino acids possess. Sometimes bulky amino acids such as valine or leucine can be used in cases where conservation of the size of mutated residues is desired. Alanine scanning is a mature technology which has been used for a long period of time. The interaction between the binding domain and the epitope or the region comprising the epitope implies that a binding domain exhibits appreciable affinity for the epitope/the region comprising the epitope on a particular protein or antigen (here: CD19 and CD3, respectively) and, generally, does not exhibit significant reactivity with proteins or antigens other than the CD19 or CD3.

Specific binding is believed to be affected by specific motifs in the amino acid sequence of the binding domain and the antigen. Thus, binding is achieved as a result of their primary, secondary and/or tertiary structure as well as the result of secondary modifications of said structures. The specific interaction of the antigen-interaction-side with its specific antigen may result in a simple binding of said side to the antigen. Moreover, the specific interaction of the antigen-interaction-side with its specific antigen may alternatively or additionally result in the initiation of a signal, e.g., due to the induction of a change of the conformation of the antigen, an oligomerization of the antigen, etc.

Variability is not evenly distributed throughout the variable domains of antibodies; it is concentrated in sub-domains of each of the heavy and light chain variable regions. These sub-domains are called “hypervariable regions” or “complementarity determining regions” (CDRs). The more conserved (i.e., non-hypervariable) portions of the variable domains are called the “framework” regions (FRM or FR) and provide a scaffold for the six CDRs in three-dimensional space to form an antigen-binding surface. The variable domains of naturally occurring heavy and light chains each comprise four FRM regions (FR1, FR2, FR3, and FR4), largely adopting a β-sheet configuration, connected by three hypervariable regions, which form loops connecting, and in some cases forming part of, the β-sheet structure. The hypervariable regions in each chain are held together in close proximity by the FRM and, with the hypervariable regions from the other chain, contribute to the formation of the antigen-binding side (see Kabat et al., loc. cit.).

Typically, CDRs form a loop structure that can be classified as a canonical structure. The term “canonical structure” refers to the main chain conformation that is adopted by the antigen binding (CDR) loops. From comparative structural studies, it has been found that five of the six antigen binding loops have only a limited repertoire of available conformations. Each canonical structure can be characterized by the torsion angles of the polypeptide backbone. Correspondent loops between antibodies may, therefore, have very similar three-dimensional structures, despite high amino acid sequence variability in most parts of the loops (Chothia and Lesk, J. Mol. Biol., 1987, 196: 901; Chothia et al., Nature, 1989, 342: 877; Martin and Thornton, J. Mol. Biol, 1996, 263: 800). Furthermore, there is a relationship between the adopted loop structure and the amino acid sequences surrounding it. The conformation of a particular canonical class is determined by the length of the loop and the amino acid residues residing at key positions within the loop, as well as within the conserved framework (i.e., outside of the loop). Assignment to a particular canonical class can therefore be made based on the presence of these key amino acid residues.

The CDR3 of the light chain and, particularly, the CDR3 of the heavy chain may constitute the most important determinants in antigen binding within the light and heavy chain variable regions. In some antibody constructs, the heavy chain CDR3 appears to constitute the major area of contact between the antigen and the antibody. In vitro selection schemes in which CDR3 alone is varied can be used to vary the binding properties of an antibody or determine which residues contribute to the binding of an antigen. Hence, CDR3 is typically the greatest source of molecular diversity within the antibody-binding side. H3, for example, can be as short as two amino acid residues or greater than 26 amino acids.

In a classical full-length antibody or immunoglobulin, each light (L) chain is linked to a heavy (H) chain by one covalent disulfide bond, while the two H chains are linked to each other by one or more disulfide bonds depending on the H chain isotype. The CH domain most proximal to VH is usually designated as CH1. The constant (“C”) domains are not directly involved in antigen binding, but exhibit various effector functions, such as antibody-dependent, cell-mediated cytotoxicity and complement activation. The Fc region of an antibody is comprised within the heavy chain constant domains and is for example able to interact with cell surface located Fc receptors.

The sequence of antibody genes after assembly and somatic mutation is highly varied, and these varied genes are estimated to encode 10¹⁰ different antibody molecules (Immunoglobulin Genes, 2^{nd} ed., eds. Jonio et al., Academic Press, San Diego, Calif, 1995). Accordingly, the immune system provides a repertoire of immunoglobulins. The term “repertoire” refers to at least one nucleotide sequence derived wholly or partially from at least one sequence encoding at least one immunoglobulin. The sequence(s) may be generated by rearrangement in vivo of the V, D, and J segments of heavy chains, and the V and J segments of light chains. Alternatively, the sequence(s) can be generated from a cell in response to which rearrangement occurs, e.g., in vitro stimulation. Alternatively, part or all of the sequence(s) may be obtained by DNA splicing, nucleotide synthesis, mutagenesis, and other methods, see, e.g., U.S. Pat. No. 5,565,332. A repertoire may include only one sequence or may include a plurality of sequences, including ones in a genetically diverse collection. An “Fc region,” “Fc domain,” or “Fc” refers to the C-terminal region of the heavy chain of an antibody. Thus, an Fc region comprises the constant region of an antibody excluding the first constant region immunoglobulin domain (e.g., CH1 or CL).

Bispecific single chain antibody constructs are known in the art and are described in WO 99/54440, Mack, J. Immunol. (1997), 158, 3965-3970, Mack, PNAS, (1995), 92, 7021-7025, Kufer, Cancer Immunol. Immunother., (1997), 45, 193-197, Löffler, Blood, (2000), 95, 6, 2098-2103, Brühl, Immunol., (2001), 166, 2420-2426, Kipriyanov, J. Mol. Biol., (1999), 293, 41-56. Techniques described for the production of single chain antibodies (see, inter alia, U.S. Pat. No. 4,946,778, Kontermann and Dübel (2010), loc. cit. and Little (2009), loc. cit.) can be adapted to produce single chain antibody constructs specifically recognizing (an) elected target(s).

Bivalent (also called divalent) or bispecific single-chain variable fragments (bi-scFvs or di-scFvs) having the format (scFv)₂ can be engineered by linking two scFv molecules (e.g. with linkers as described hereinbefore). If these two scFv molecules have the same binding specificity, the resulting (scFv)₂ molecule will preferably be called bivalent (i.e. it has two valences for the same target epitope). If the two scFv molecules have different binding specificities, the resulting (scFv)₂ molecule will preferably be called bispecific. The linking can be done by producing a single peptide chain with two VH regions and two VL regions, yielding tandem scFvs (see e.g., Kufer P. et al., (2004) Trends in Biotechnology 22(5):238-244). Another possibility is the creation of scFv molecules with linker peptides that are too short for the two variable regions to fold together (e.g. about five amino acids), forcing the scFvs to dimerize. This type is known as diabodies (see e.g. Hollinger, Philipp et al., (July 1993) Proceedings of the National Academy of Sciences of the United States of America 90 (14): 6444-8).

The monoclonal antibodies herein also include camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079, which are hereby incorporated by reference in their entireties). In one aspect, provided herein are single domain antibodies comprising two VH domains with modifications such that single domain antibodies are formed.

T cells or T lymphocytes are a type of lymphocyte (itself a type of white blood cell) that play a central role in cell-mediated immunity. There are several subsets of T cells, each with a distinct function. T cells can be distinguished from other lymphocytes, such as B cells and NK cells, by the presence of a T cell receptor (TCR) on the cell surface. The TCR is responsible for recognizing antigens bound to major histocompatibility complex (MHC) molecules and is composed of two different protein chains. In 95% of the T cells, the TCR consists of an alpha (α) and beta (β) chain. When the TCR engages with antigenic peptide and MHC (peptide/MHC complex), the T lymphocyte is activated through a series of biochemical events mediated by associated enzymes, co-receptors, specialized adaptor molecules, and activated or released transcription factors.

The CD3 receptor complex is a protein complex and is composed of four chains. In mammals, the complex contains a CD3γ (gamma) chain, a CD3δ (delta) chain, and two CD3ε (epsilon) chains. These chains associate with the T cell receptor (TCR) and the so-called ζ (zeta) chain to form the T cell receptor CD3 complex and to generate an activation signal in T lymphocytes. The CD3γ (gamma), CD3δ (delta), and CD3ε (epsilon) chains are highly related cell-surface proteins of the immunoglobulin superfamily containing a single extracellular immunoglobulin domain. The intracellular tails of the CD3 molecules contain a single conserved motif known as an immunoreceptor tyrosine-based activation motif or ITAM for short, which is essential for the signaling capacity of the TCR. The CD3 epsilon molecule is a polypeptide which in humans is encoded by the CD3E gene which resides on chromosome 11. An epitope of CD3 epsilon that has been investigated is comprised within amino acid residues 1-27 of the human CD3 epsilon extracellular domain. It is envisaged that binding molecules or antibody constructs according to the present invention typically and advantageously show less unspecific T cell activation.

The redirected lysis of target cells via the recruitment of T cells by a multispecific, e.g., bispecific construct, involves cytolytic synapse formation and delivery of perforin and granzymes. The engaged T cells are capable of serial target cell lysis, and are not affected by immune escape mechanisms interfering with peptide antigen processing and presentation, or clonal T cell differentiation. See, for example, WO 2007/042261.

Cytotoxicity mediated by antibody constructs of the invention can be measured in various ways. Effector cells can be e.g. stimulated enriched (human) CD8 positive T cells or unstimulated (human) peripheral blood mononuclear cells (PBMC). If the target cells are of macaque origin or express or are transfected with macaque CD19 which is bound by the first domain, the effector cells should also be of macaque origin such as a macaque T cell line, e.g. 4119LnPx. The target cells should express (at least the extracellular domain of) CD19, e.g. human or macaque CD19. Target cells can be a cell line (such as CHO) which is stably or transiently transfected with CD19, e.g., human or macaque CD19. Alternatively, the target cells can be a CD19 positive natural expresser cell line such as the human multiple myeloma cell line L363 or NCI-H929. Usually EC₅₀ values are expected to be lower with target cell lines expressing higher levels of CD19 on the cell surface. The effector to target cell (E:T) ratio is usually about 10:1, but can also vary. Cytotoxic activity of CD19×CD3 bispecific antibody constructs can be measured in a ⁵¹Cr-release assay (incubation time of about 18 hours) or in a in a FACS-based cytotoxicity assay (incubation time of about 48 hours). Modifications of the assay incubation time (cytotoxic reaction) are also possible. Other methods of measuring cytotoxicity are well-known to the skilled person and comprise MTT or MTS assays, ATP-based assays including bioluminescent assays, the sulforhodamine B (SRB) assay, WST assay, clonogenic assay and the ECIS technology.

Covalent modifications of the constructs are also included within the scope of this invention, and are generally, but not always, done post-translationally. For example, several types of covalent modifications of the antibody construct are introduced into the molecule by reacting specific amino acid residues of the antibody construct with an organic derivatizing agent that is capable of reacting with selected side chains or the N- or C-terminal residues.

Cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(5-imidozoyl)propionic acid, chloroacetyl phosphate, N-alkylmaleimides, 3-nitro-2-pyridyl disulfide, methyl 2-pyridyl disulfide, β-chloromercuribenzoate, 2-chloromercuri-4-nitrophenol, or chloro-7-nitrobenzo-2-oxa-1,3-diazole.

Histidyl residues are derivatized by reaction with diethylpyrocarbonate at pH 5.5-7.0 because this agent is relatively specific for the histidyl side chain. Para-bromophenacyl bromide also is useful; the reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing alpha-amino-containing residues include imidoesters such as methyl picolinimidate; pyridoxal phosphate; pyridoxal; chloroborohydride; trinitrobenzenesulfonic acid; O-methylisourea; 2,4-pentanedione; and transaminase-catalyzed reaction with glyoxylate.

Arginyl residues are modified by reaction with one or several conventional reagents, among them phenylglyoxal, 2,3-butanedione, 1,2-cyclohexanedione, and ninhydrin. Derivatization of arginine residues requires that the reaction be performed in alkaline conditions because of the high pKa of the guanidine functional group. Furthermore, these reagents may react with the groups of lysine as well as the arginine epsilon-amino group.

The specific modification of tyrosyl residues may be made, with particular interest in introducing spectral labels into tyrosyl residues by reaction with aromatic diazonium compounds or tetranitromethane. Most commonly, N-acetylimidizole and tetranitromethane are used to form 0-acetyl tyrosyl species and 3-nitro derivatives, respectively. Tyrosyl residues are iodinated using ¹²⁵I or ¹³¹I to prepare labeled proteins for use in radioimmunoassay, the chloramine T method described above being suitable.

Carboxyl side groups (aspartyl or glutamyl) are selectively modified by reaction with carbodiimides (R′—N═C═N—R′), where R and R′ are optionally different alkyl groups, such as 1-cyclohexyl-3-(2-morpholinyl-4-ethyl) carbodiimide or 1-ethyl-3-(4-azonia-4,4-dimethylpentyl) carbodiimide. Furthermore, aspartyl and glutamyl residues are converted to asparaginyl and glutaminyl residues by reaction with ammonium ions.

Derivatization with bifunctional agents is useful for crosslinking the antibody constructs of the present invention to a water-insoluble support matrix or surface for use in a variety of methods. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), and bifunctional maleimides such as bis-N-maleimido-1,8-octane. Derivatizing agents such as methyl-3-[(p-azidophenyl)dithio]propioimidate yield photoactivatable intermediates that are capable of forming crosslinks in the presence of light.

Alternatively, reactive water-insoluble matrices such as cyanogen bromide-activated carbohydrates and the reactive substrates as described in U.S. Pat. Nos. 3,969,287; 3,691,016; 4,195,128; 4,247,642; 4,229,537; and 4,330,440 are employed for protein immobilization.

Glutaminyl and asparaginyl residues are frequently deamidated to the corresponding glutamyl and aspartyl residues, respectively. Alternatively, these residues are deamidated under mildly acidic conditions. Either form of these residues falls within the scope of this invention.

Other modifications include hydroxylation of proline and lysine, phosphorylation of hydroxyl groups of seryl or threonyl residues, methylation of the α-amino groups of lysine, arginine, and histidine side chains (T. E. Creighton, Proteins: Structure and Molecular Properties, W. H. Freeman & Co., San Francisco, 1983, pp. 79-86), acetylation of the N-terminal amine, and amidation of any C-terminal carboxyl group.

Another type of covalent modification of the antibody constructs included within the scope of this invention comprises altering the glycosylation pattern of the protein. As is known in the art, glycosylation patterns can depend on both the sequence of the protein (e.g., the presence or absence of particular glycosylation amino acid residues, discussed below), or the host cell or organism in which the protein is produced. Particular expression systems are discussed below.

Glycosylation of polypeptides is typically either N-linked or O-linked. N-linked refers to the attachment of the carbohydrate moiety to the side chain of an asparagine residue. The tri-peptide sequences asparagine-X-serine and asparagine-X-threonine, where X is any amino acid except proline, are the recognition sequences for enzymatic attachment of the carbohydrate moiety to the asparagine side chain. Thus, the presence of either of these tri-peptide sequences in a polypeptide creates a potential glycosylation site. O-linked glycosylation refers to the attachment of one of the sugars N-acetylgalactosamine, galactose, or xylose, to a hydroxyamino acid, most commonly serine or threonine, although 5-hydroxyproline or 5-hydroxylysine may also be used.

Addition of glycosylation sites to the antibody construct is conveniently accomplished by altering the amino acid sequence such that it contains one or more of the above-described tri-peptide sequences (for N-linked glycosylation sites). The alteration may also be made by the addition of, or substitution by, one or more serine or threonine residues to the starting sequence (for O-linked glycosylation sites). For ease, the amino acid sequence of an antibody construct is preferably altered through changes at the DNA level, particularly by mutating the DNA encoding the polypeptide at preselected bases such that codons are generated that will translate into the desired amino acids.

Another means of increasing the number of carbohydrate moieties on the antibody construct is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, 1981, CRC Crit. Rev. Biochem., pp. 259-306.

Removal of carbohydrate moieties present on the starting binding molecule or antibody construct may be accomplished chemically or enzymatically. Chemical deglycosylation requires exposure of the protein to the compound trifluoromethanesulfonic acid, or an equivalent compound. This treatment results in the cleavage of most or all sugars except the linking sugar (N-acetylglucosamine or N-acetylgalactosamine), while leaving the polypeptide intact. Chemical deglycosylation is described by Hakimuddin et al., 1987, Arch. Biochem. Biophys. 259:52 and by Edge et al., 1981, Anal. Biochem. 118:131. Enzymatic cleavage of carbohydrate moieties on polypeptides can be achieved by the use of a variety of endo- and exo-glycosidases as described by Thotakura et al., 1987, Meth. Enzymol. 138:350. Glycosylation at potential glycosylation sites may be prevented by the use of the compound tunicamycin as described by Duskin et al., 1982, J. Biol. Chem. 257:3105. Tunicamycin blocks the formation of protein-N-glycoside linkages.

Other modifications of the binding molecule or antibody construct are also contemplated herein. For example, another type of covalent modification of the antibody construct comprises linking the antibody construct to various non-proteinaceous polymers, including, but not limited to, various polyols such as polyethylene glycol, polypropylene glycol, polyoxyalkylenes, or copolymers of polyethylene glycol and polypropylene glycol, in the manner set forth in U.S. Pat. Nos. 4,640,835; 4,496,689; 4,301,144; 4,670,417; 4,791,192 or 4,179,337. In addition, as is known in the art, amino acid substitutions may be made in various positions within the antibody construct, e.g., in order to facilitate the addition of polymers such as PEG.

In some embodiments, the covalent modification of the binding molecules or antibody constructs of the invention comprises the addition of one or more labels. The labelling group may be coupled to the antibody construct via spacer arms of various lengths to reduce potential steric hindrance. Various methods for labelling proteins are known in the art and can be used in performing the present invention. The term “label” or “labelling group” refers to any detectable label. In general, labels fall into a variety of classes, depending on the assay in which they are to be detected—the following examples include, but are not limited to:
a) isotopic labels, which may be radioactive or heavy isotopes, such as radioisotopes or radionuclides
   (e.g ³H, ¹⁴C, ¹⁵N, ³⁵S, ⁸⁹Zr, ⁹⁰Y, ⁹⁹Tc, ¹¹¹In, ¹²⁵I, ¹³¹I)
b) magnetic labels (e.g., magnetic particles)
c) redox active moieties
d) optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluores or proteinaceous fluores
e) enzyinatic groups (e.g. horseradish peroxidase, β-galactosidase, luciferase, alkaline phosphatase)
f) biotinylated groups
g) predetermined polypeptide epitopes recognized by a secondary reporter (e.g., leucine zipper pair sequences, binding sides for secondary and bodies, metal binding domains, epitope tags, etc.)

The binding molecule or antibody construct of the invention may also comprise additional domains, which are e.g. helpful in the isolation of the molecule or relate to an adapted pharmacokinetic profile of the molecule. Domains helpful for the isolation of an antibody construct may be selected from peptide motives or secondarily introduced moieties, which can be captured in an isolation method, e.g., an isolation column. Non-limiting embodiments of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g., StrepII-tag) and His-tag. All herein disclosed binding molecules or antibody constructs may comprise a His-tag domain, which is generally known as a repeat of consecutive His residues in the amino acid sequence of a molecule, preferably of five, and more preferably of six His residues (hexa-histidine). The His-tag may be located e.g., at the N- or C-terminus of the antibody construct, preferably it is located at the C-terminus. Most preferably, a hexa-histidine tag (HHHHHH; SEQ ID NO: 222) is linked via peptide bond to the C-terminus of the antibody construct according to the invention. Additionally, a conjugate system of PLGA-PEG-PLGA may be combined with a poly-histidine tag for sustained release application and improved pharmacokinetic profile.

Amino acid sequence modifications of the binding molecules or antibody constructs described herein are also contemplated. For example, it may be desirable to improve the binding affinity and/or other biological properties of the binding molecule or antibody construct. Amino acid sequence variants of the binding molecule or antibody constructs are prepared by introducing appropriate nucleotide changes into the binding molecule or antibody constructs nucleic acid, or by peptide synthesis. All of the below described amino acid sequence modifications should result in an binding molecule or antibody construct which still retains the desired biological activity (e.g., binding to CD19 and to CD3) of the unmodified parental molecule.

The sites of greatest interest for substitutional mutagenesis include (but are not limited to) the CDRs of the heavy and/or light chain, in particular the hypervariable regions, but FR alterations in the heavy and/or light chain are also contemplated. The substitutions are preferably conservative substitutions as described herein. Preferably, 1, 2, 3, 4, 5, 6, 7, 8, 9, or 10 amino acids may be substituted in a CDR, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR. For example, if a CDR sequence encompasses 6 amino acids, it is envisaged that one, two or three of these amino acids are substituted. Similarly, if a CDR sequence encompasses 15 amino acids it is envisaged that one, two, three, four, five or six of these amino acids are substituted.

A useful method for identification of certain residues or regions of the binding molecules or antibody constructs that are preferred locations for mutagenesis is called “alanine scanning mutagenesis” as described by Cunningham and Wells in Science, 244: 1081-1085 (1989). Here, a residue or group of target residues within the antibody construct is/are identified (e.g., charged residues such as arg, asp, his, lys, and glu) and replaced by a neutral or negatively charged amino acid (most preferably alanine or polyalanine) to affect the interaction of the amino acids with the epitope.

Those amino acid locations demonstrating functional sensitivity to the substitutions are then refined by introducing further or other variants at, or for, the sites of substitution. Thus, while the site or region for introducing an amino acid sequence variation is predetermined, the nature of the mutation per se needs not to be predetermined. For example, to analyze or optimize the performance of a mutation at a given site, alanine scanning or random mutagenesis may be conducted at a target codon or region, and the expressed antibody construct variants are screened for the optimal combination of desired activity. Techniques for making substitution mutations at predetermined sites in the DNA having a known sequence are well known, for example, M13 primer mutagenesis and PCR mutagenesis. Screening of the mutants is done using assays of antigen binding activities, such as CD19 or CD3 binding.

Generally, if amino acids are substituted in one or more or all of the CDRs of the heavy and/or light chain, it is preferred that the then-obtained “substituted” sequence is at least 60% or 65%, more preferably 70% or 75%, even more preferably 80% or 85%, and particularly preferably 90% or 95% identical to the “original” CDR sequence. This means that it is dependent of the length of the CDR to which degree it is identical to the “substituted” sequence. For example, a CDR having 5 amino acids is preferably 80% identical to its substituted sequence in order to have at least one amino acid substituted. Accordingly, the CDRs of the antibody construct may have different degrees of identity to their substituted sequences, e.g., CDR-L1 may have 80%, while CDR-L3 may have 90%.

Preferred substitutions (or replacements) are conservative substitutions. However, any substitution (including non-conservative substitution or one or more from the “exemplary substitutions” described herein) is envisaged as long as the antibody construct retains its capability to bind to CD19 via the first domain and to CD3 epsilon via the second domain and/or its CDRs have an identity to the then substituted sequence (at least 60% or 65%, more preferably 70% or 75%, even more preferably 80% or 85%, and particularly preferably 90% or 95% identical to the “original” CDR sequence).

Also provided are “conservative sequence modifications” of the sequences set forth herein, e.g., amino acid sequence modifications which do not abrogate the binding of the antibody encoded by the nucleotide sequence or containing the amino acid sequence, to the antigen. Such conservative sequence modifications include conservative nucleotide and amino acid substitutions, as well as nucleotide and amino acid additions and deletions. For example, modifications can be introduced into a sequence by standard techniques known in the art, such as site-directed mutagenesis and PCR-mediated mutagenesis. Conservative amino acid substitutions include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-CD19 or anti-CD3 antibody is preferably replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). Alternatively, in another aspect, mutations can be introduced randomly along all or part of an anti-CD19 or anti-CD3 antibody antibody coding sequence, such as by saturation mutagenesis, and the resulting modified antibodies can be screened for binding activity.

The nucleic acid and protein sequences described herein can further be used as a “query sequence” to perform a search against public databases to, for example, identify related sequences. Such searches can be performed using the NBLAST and XBLAST programs (version 2.0) of Altschul, et al. (1990) J. Mol. Biol. 215:403-10. BLAST nucleotide searches can be performed with the NBLAST program, score=100, wordlength=12 to obtain nucleotide sequences homologous to the nucleic acid molecules described herein. BLAST protein searches can be performed with the XBLAST program, score=50, wordlength=3 to obtain amino acid sequences homologous to the protein molecules described herein. To obtain gapped alignments for comparison purposes, Gapped BLAST can be utilized as described in Altschul et al., (1997) Nucleic Acids Res. 25(17):3389-3402. When utilizing BLAST and Gapped BLAST programs, the default parameters of the respective programs (e.g., XBLAST and NBLAST) can be used. See www.ncbi.nlm.nih.gov.

### I. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising,” “consisting essentially of,” and “consisting of” may be optionally replaced with either of the other two terms, thus describing alternative aspects of the scope of the subject matter. The invention illustratively described herein suitably may be practiced in the absence of any element or elements, limitation or limitations which is not specifically disclosed herein.

As used herein, the singular forms “a,” “an,” and “the” include plural referents unless the context clearly dictates otherwise. The use of “or” or “and” means “and/or” unless stated otherwise. Furthermore, use of the term “including” as well as other forms, such as “include,” “includes,” and “included,” is not limiting.

The term “about” as used herein when referring to a measurable value such as an amount, a temporal duration and the like, encompasses variations of up to ±10% from the specified value. Unless otherwise indicated, all numbers expressing e.g., quantities of ingredients or properties (e.g., molecular weight, reaction conditions) described herein are to be understood as being modified by the term “about”.

As used herein, “and/or” is to be taken as specific disclosure of each of the two specified features or components with or without the other. Thus, the term “and/or” as used in a phrase such as “A and/or B” includes “A and B,” “A or B,” “A” alone, and “B” alone. Likewise, the term “and/or” as used in a phrase such as “A, B, and/or C” encompasses each of the following: A, B, and C; A, B, or C; A or C; A or B; B or C; A and C; A and B; B and C; A alone; B alone; and C alone. I ma

As used herein, recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3% are expressly enumerated in this specification. These are only examples of what is specifically intended, and all possible combinations of numerical values between and including the lowest value and the highest value enumerated are to be considered to be expressly stated in this disclosure.

Table 1 lists exemplary position boundaries of CDR-L1, CDR-L2, CDR-L3 and CDR-H1, CDR-H2, CDR-H3 as identified by Kabat, Chothia, AbM, and Contact schemes, respectively. For CDR-H1, residue numbering is listed using both the Kabat and Chothia numbering schemes. FRs are located between CDRs, for example, with FR-L1 located before CDR-L1, FR-L2 located between CDR-L1 and CDR-L2, FR-L3 located between CDR-L2 and CDR-L3 and so forth. It is noted that because the shown Kabat numbering scheme places insertions at H35A and H35B, the end of the Chothia CDR-H1 loop when numbered using the shown Kabat numbering convention varies between H32 and H34, depending on the length of the loop.

TABLE 1
Boundaries of CDRs according to various numbering schemes.

| CDR | Kabat | Chothia | AbM | Contact | IMGT |
|---|---|---|---|---|---|
| CDR-L1 | L24 - - - L34 | L24 - - - L34 | L24 - - - L34 | L30 - - - L36 | L27-L38 |
| CDR-L2 | L50 - - - L56 | L50 - - - L56 | L50 - - - L56 | L46 - - - L55 | L56-L65 |
| CDR-L3 | L89 - - - L97 | L89 - - - L97 | L89 - - - L97 | L89 - - - L96 | L105-L117 |
| CDR-H1 | H31 - - - H35B | H26 - - - H32.34 | H26 - - - H35B | H30 - - - H35B | H27-H38 |
| (Kabat Numbering¹) | | | | | |
| CDR-H1 | H31 - - - H35 | H26 - - - H32 | H26 - - - H35 | H30 - - - H35 | H27-H38 |
| (Chothia Numbering²) | | | | | |
| CDR-H2 | H50 - - - H65 | H52 - - - H56 | H50 - - - H58 | H47 - - - H58 | H56-H65 |
| CDR-H3 | H95 - - - H102 | H95 - - - H102 | H95 - - - H102 | H93 - - - H101 | H105-H117 |

¹Kabat et al. (1991), “Sequences of Proteins of Immunological Interest,” 5th Ed. Public Health Service, National Institutes of Health, Bethesda, MD
²Al-Lazikani et al., (1997) JMB 273,927-948

Thus, unless otherwise specified, a “CDR” or “complementary determining region,” or individual specified CDRs (e.g., CDR-H1, CDR-H2, CDR-H3), of a given antibody or region thereof, such as a variable region thereof, should be understood to encompass a (or the specific) complementary determining region as defined by any of the aforementioned schemes, or other known schemes. For example, where it is stated that a particular CDR (e.g., a CDR-H3) contains the amino acid sequence of a corresponding CDR in a given V_{H} or V_{L} region amino acid sequence, it is understood that such a CDR has a sequence of the corresponding CDR (e.g., CDR-H3) within the variable region, as defined by any of the aforementioned schemes, or other known schemes. In some embodiments, the provided binding molecule, comprises a CDR-H1, a CDR-H2, and a CDR-H3 as contained within a given VH region amino acid sequence and defined by any of the aforementioned schemes, such as Kabat, Chothia, AbM, IgBLAST, IMGT, or Contact method, or other known schemes, and a CDR-L1, a CDR-L2, and a CDR-L3 as contained within a given V_{L} region amino acid sequence and defined by any of the aforementioned schemes, such as Kabat, Chothia, AbM, IgBLAST, IMGT, or Contact method, or other known schemes. In some embodiments, specific CDR sequences are specified. In some embodiments, specific CDR sequences are specified. Exemplary CDR sequences of provided antibodies are described using various numbering schemes (see e.g. Table 1), although it is understood that a provided antibody can include CDRs as described according to any of the other aforementioned numbering schemes or other known numbering schemes.

As used herein, the term “framework” or “FR” residues refers to those variable domain residues other than the hypervariable region residues defined herein as CDR residues. Unless otherwise specified, a FR or individual specified FR(s) (e.g., FR-H1, FR-H2, FR-H3, FR-H4), of a given antibody or region thereof, such as a variable region thereof, should be understood to encompass a (or the specific) framework region as defined by any of the known schemes. In some instances, the scheme for identification of a particular CDR, FR, or FRs or CDRs is specified, such as the CDR as defined by the Kabat, Chothia, AbM, IgBLAST, IMGT, or Contact method, or other known schemes. In other cases, the particular amino acid sequence of a CDR or FR is given.

The term “variable region” or “variable domain” refers to the domain of an antibody heavy or light chain that is involved in binding the antibody to antigen. The variable regions of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W.H. Freeman and Co., page 91 (2007). A single V_{H} or V_{L} domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

Among the provided antibodies are antibody fragments. An “antibody fragment” or “antigen-binding fragment” refers to a molecule other than an intact or full-length antibody that comprises a portion of an intact or full-length antibody that binds the antigen to which the intact antibody binds. The term thus refers to one or more fragments of an antibody that retain the ability to specifically bind to an antigen (e.g., human CD3 or human CD19). It has been shown that the antigen-binding function of an antibody can be performed by fragments of a full-length antibody. Examples of antibody fragments include but are not limited to Fv, Fab, Fab′, Fab′-SH, F(ab′)₂; diabodies; linear antibodies; VH regions, single-chain antibody molecules such as scFvs and single-domain antibodies comprising only the VH region; and multispecific antibodies formed from antibody fragments. In some embodiments, the antibody is or comprises an antibody fragment comprising a VH and a VL region. In some embodiments, the antibodies are single-chain antibody fragments comprising a heavy chain variable VH region and/or a VL region, such as Fvs or scFvs. In some embodiments, the antibodies are Fab composed of an entire light chain (i.e. VL region and a constant light chain (CL)) and the VH region and the first constant domain of one heavy chain (CH1).

Antibody fragments can be made by various techniques, including but not limited to proteolytic digestion of an intact antibody as well as production by recombinant host cells. For instance, papain digestion of full-length antibodies produces two identical antigen-binding fragments, called “Fab” fragments, and a residual “Fc” fragment, a designation reflecting the ability to crystallize readily. Pepsin treatment of an antibody yields a single large F(ab′)2 fragment which roughly corresponds to two disulfide linked Fab fragments having different antigen-binding activity and is still capable of cross-linking antigen. Fab′ fragments differ from Fab fragments by having a few additional residues at the carboxy terminus of the CH1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known. In some embodiments, the antibodies are recombinantly-produced fragments. For instance, the chains of an antibody or antigen-binding fragment can be incorporated into an expression vector and expressed from a host cell to produce the antibody. In some instances, a recombinantly-produced antibody fragments also can include fragments comprising arrangements that do not occur naturally, such as those with two or more antibody regions or chains joined by synthetic linkers, e.g., peptide linkers, and/or that are may not be produced by enzyme digestion of a naturally-occurring intact antibody. In some aspects, the antibody fragments are scFvs. For example, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. Antigen-binding portions can be produced by recombinant DNA techniques, or by enzymatic or chemical cleavage of intact immunoglobulins.

The term “human antibody”, “human antibody construct” and “human binding domain” includes antibodies, antibody constructs and binding domains having antibody regions such as variable and constant regions or domains which correspond substantially to human germline immunoglobulin sequences known in the art, including, for example, those described by Kabat et al. (1991) (loc. cit.). The human antibodies, antibody constructs or binding domains of the invention may include amino acid residues not encoded by human germline immunoglobulin sequences (e.g., mutations introduced by random or side-specific mutagenesis in vitro or by somatic mutation in vivo), for example in the CDRs, and in particular, in CDR3. The human antibodies, antibody constructs or binding domains can have at least one, two, three, four, five, or more positions replaced with an amino acid residue that is not encoded by the human germline immunoglobulin sequence. The definition of human antibodies, antibody constructs and binding domains as used herein, however, also contemplates “fully human antibodies”, which include only non-artificially and/or genetically altered human sequences of antibodies as those can be derived by using technologies or systems such as the Xenomouse. Preferably, a “fully human antibody” does not include amino acid residues not encoded by human germline immunoglobulin sequences.

Furthermore, the definition of the term “antibody construct” includes monovalent, bivalent and polyvalent/multivalent constructs and, thus, bispecific constructs, the latter which specifically bind to only two antigenic structure, as well as polyspecific/multispecific constructs, which specifically bind more than two antigenic structures, e.g. three, four or more, through distinct binding domains. Moreover, the definition of the term “antibody construct” includes molecules consisting of only one polypeptide chain as well as molecules consisting of more than one polypeptide chain, which chains can be either identical (homodimers, homotrimers or homo oligomers) or different (heterodimer, heterotrimer or heterooligomer). Examples for the above identified antibodies and variants or derivatives thereof are described inter alia in Harlow and Lane, Antibodies a laboratory manual, CSHL Press (1988) and Using Antibodies: a laboratory manual, CSHL Press (1999), Kontermann and Dubel, Antibody Engineering, Springer, 2nd ed. 2010 and Little, Recombinant Antibodies for Immunotherapy, Cambridge University Press 2009.

The term “bispecific” as used herein refers to an antibody construct which is that comprises at least a first binding domain and a second binding domain, wherein the first binding domain binds to one antigen or target (e.g., CD19 as disclosed herein), and the second binding domain binds to another antigen or target (e.g, CD3 as disclosed herein). Accordingly, antibody constructs according to the invention comprise specificities for at least two different antigens or targets. A bispecific antibody can be a monoclonal antibody that has binding specificity for two different epitopes. Bispecific antibodies can be prepared as full length antibodies or antibody fragments. Given that the antibody constructs are (at least) bispecific, they do not occur naturally and they are markedly different from naturally occurring products. A “bispecific” antibody construct or immunoglobulin is hence an artificial hybrid antibody or immunoglobulin having at least two distinct binding sides with different specificities. Bispecific antibody constructs can be produced by a variety of methods including fusion of hybridomas or linking of Fab′ fragments. See, e.g., Songsivilai & Lachmann, Clin. Exp. Immunol. 79:315-321 (1990).

The term “target cell surface antigen” refers to an antigenic structure expressed by a cell and which is present at the cell surface such that it is accessible for an antibody construct as described herein. It may be a protein, preferably the extracellular portion of a protein, or a carbohydrate structure, preferably a carbohydrate structure of a protein, such as a glycoprotein.

As used herein, “administering” refers to the physical introduction of a CD19 binding molecule, such as an anti-CD19 antibody or an anti-CD19 and anti-CD3 bispecific antibody, including antibody fragments thereof, or a nucleic acid or expression vector as described herein encoding the same, alone or in combination with another therapeutic agent to a subject, using any of the various methods and delivery systems known to those skilled in the art. Preferred routes of administration for antibodies described herein include intravenous, intraperitoneal, intramuscular, subcutaneous, spinal or other parenteral routes of administration, for example by injection or infusion. The phrase “parenteral administration” as used herein means modes of administration other than enteral and topical administration, usually by injection, and includes, without limitation, intravenous, intraperitoneal, intramuscular, intraarterial, intrathecal, intra-lymphatic, intralesional, intracapsular, intra-orbital, intracardiac, intradermal, transtracheal, subcutaneous, subcuticular, intraarticular, subcapsular, subarachnoid, intraspinal, epidural and intrasternal injection and infusion, as well as in vivo electroporation. Alternatively, an antibody described herein can be administered via a non-parenteral route, such as a topical, epidermal or mucosal route of administration, for example, intranasally, orally, vaginally, rectally, sublingually or topically. Administering can also be performed, for example, once, a plurality of times, and/or over one or more extended periods.

The terms “treat,” “treating,” and “treatment,” as used herein, refer to any type of intervention or process performed on, or administering an active agent (e.g., an anti-CD19 binding molecule, such as an anti-CD19 antibody or an anti-CD19 and anti-CD3 bispecific antibody, including antibody fragments thereof) to, the subject with the objective of preventing, reversing, alleviating, ameliorating, inhibiting, or slowing down or preventing the progression, development, severity or recurrence of a symptom, complication, condition or biochemical indicia associated with a disease. Treatment can be of a subject having a disease or a subject who does not have a disease (e.g., for prophylaxis).

As used herein, the term “subject” includes any human or non-human animal. The term “non-human animal” includes all vertebrates, e.g., a mammals and non-mammals, such as non-human primates, sheep, cats, dogs, cows, chickens, amphibians, and reptiles.

The term “patient” includes human and other mammalian subjects that receive either prophylactic or therapeutic treatment.

The terms “detection” or “detected”, as used herein refer to qualitative and/or quantitative detection (measuring levels) with or without reference to a control.

The term “diagnosing”, as used herein, means the determination of the nature of a medical condition intended to identify a pathology which affects the subject from a number of collected data.

The terms “(specifically) binds to”, (specifically) recognizes”, “is (specifically) directed to”, and “(specifically) reacts with” mean in accordance with this invention that a binding domain interacts or specifically interacts with a given epitope or a given target side on the target molecules (antigens), e.g., CD19 and/or CD3 as disclosed herein.

The term “epitope” refers to a side on an antigen to which a binding domain, such as an antibody or immunoglobulin, or a derivative, fragment or variant of an antibody or an immunoglobulin, specifically binds. An “epitope” is antigenic and thus the term epitope is sometimes also referred to herein as “antigenic structure” or “antigenic determinant”. Thus, the binding domain is an “antigen interaction side”. Said binding/interaction is also understood to define a “specific recognition”.

“Epitopes” can be formed both by contiguous amino acids or non-contiguous amino acids juxtaposed by tertiary folding of a protein. A “linear epitope” is an epitope where an amino acid primary sequence comprises the recognized epitope. A linear epitope typically includes at least 3 or at least 4, and more usually, at least 5 or at least 6 or at least 7, for example, about 8 to about 10 amino acids in a unique sequence.

A “conformational epitope”, in contrast to a linear epitope, is an epitope wherein the primary sequence of the amino acids comprising the epitope is not the sole defining component of the epitope recognized (e.g., an epitope wherein the primary sequence of amino acids is not necessarily recognized by the binding domain). With regard to recognition of conformational epitopes, the binding domain recognizes a three-dimensional structure of the antigen, preferably a peptide or protein or fragment thereof (in the context of the present invention, the antigenic structure for one of the binding domains is comprised within the target cell surface antigen protein). For example, when a protein molecule folds to form a three-dimensional structure, certain amino acids and/or the polypeptide backbone forming the conformational epitope become juxtaposed enabling the antibody to recognize the epitope. Methods of determining the conformation of epitopes include, but are not limited to, x-ray crystallography, two-dimensional nuclear magnetic resonance (2D-NMR) spectroscopy and site-directed spin labelling and electron paramagnetic resonance (EPR) spectroscopy.

“Appreciable affinity” includes binding with an affinity of about 10⁻⁶M (KD) or stronger. Preferably, binding is considered specific when the binding affinity is about 10⁻¹² to 10⁻⁸M, 10⁻¹² to 10⁻⁹M, 10⁻¹² to 10⁻¹⁰M, 10⁻¹¹ to 10⁻⁸M, preferably of about 10⁻¹¹ to 10⁻⁹M. Whether a binding domain specifically reacts with or binds to a target can be tested readily by, inter alia, comparing the reaction of said binding domain with a target protein or antigen with the reaction of said binding domain with proteins or antigens other than the CD19 or CD3. Preferably, a binding domain of the invention does not essentially or substantially bind to proteins or antigens other than CD19 or CD3 (i.e., the first binding domain is not capable of binding to proteins other than CD19 and the second binding domain is not capable of binding to proteins other than CD3). It is an envisaged characteristic of the antibody constructs according to the present invention to have superior affinity characteristics in comparison to other HLE formats. Such a superior affinity, in consequence, suggests a prolonged half-life in vivo. The longer half-life of the antibody constructs according to the present invention may reduce the duration and frequency of administration which typically contributes to improved patient compliance. This is of particular importance as the antibody constructs of the present invention are particularly beneficial for highly weakened or even treatment resistant B cell disorder patients.

The term “does not essentially/substantially bind” or “is not capable of binding” means that a binding domain of the present invention does not bind a protein or antigen other than the CD19 or CD3, i.e., does not show reactivity of more than 30%, preferably not more than 20%, more preferably not more than 10%, particularly preferably not more than 9%, 8%, 7%, 6% or 5% with proteins or antigens other than CD19 or CD3, whereby binding to the CD19 or CD3, respectively, is set to be 100%.

As used herein, “isotype” refers to the antibody class (e.g., IgG (including IgG1, IgG2, IgG3, and IgG4), IgM, IgA (including IgA1 and IgA2), IgD, and IgE antibody) that is encoded by the heavy chain constant region genes of the antibody.

An antibody may be from any of the commonly known isotypes, including but not limited to IgA, secretory IgA, IgG and IgM. The IgG isotype is divided in subclasses in certain species: IgG1, IgG2, IgG3 and IgG4 in humans, and IgG1, IgG2a, IgG2b and IgG3 in mice. Immunoglobulins, e.g., IgG1, exist in several allotypes, which differ from each other in at most a few amino acids.

As used herein, the term “allotype” refers to naturally occurring variants within a specific isotype group, where the variants differ in a few amino acids. Anti-antibodies described herein can be of any allotype. The mutation of certain residues would modulate (i.e., eliminate or decrease) the binding of the antibodies to Fcγ receptors and/or C1q, and thus reduce activator efficacy of the Fc domain of the IgG component of an antibody.

For nucleic acids, the term “substantial homology” indicates that two nucleic acids, or designated sequences thereof, when optimally aligned and compared, are identical, with appropriate nucleotide insertions or deletions, in at least about 80% of the nucleotides, usually at least about 80% to 85%, 85% to 90% or 90% to 95%, and more preferably at least about 98% to 99.5% of the nucleotides. Alternatively, substantial homology exists when the segments will hybridize under selective hybridization conditions, to the complement of the strand. For polypeptides, the term “substantial homology” indicates that two polypeptides, or designated sequences thereof, when optimally aligned and compared, are identical, with appropriate amino acid insertions or deletions, in at least about 80% of the amino acids, usually at least about 80% to 85%, 85% to 90%, 90% to 95%, and more preferably at least about 98% to 99.5% of the amino acids.

The percent identity between two sequences is a function of the number of identical positions shared by the sequences (i.e., % identity=# of identical positions/total # of positions ×100), considering the number of gaps, and the length of each gap, which need to be introduced for optimal alignment of the two sequences. The comparison of sequences and determination of percent identity between two sequences can be accomplished using a mathematical algorithm, as described in the non-limiting examples below.

The percent identity between two nucleotide sequences can be determined using the GAP program in the GCG software package (available at http://www.gcg.com), using a NWSgapdna.CMP matrix and a gap weight of 40, 50, 60, 70, or 80 and a length weight of 1, 2, 3, 4, 5, or 6. The percent identity between two nucleotide or two amino acid sequences can also be determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4. In addition, the percent identity between two amino acid sequences can be determined using the Needleman and Wunsch (J. Mol. Biol. (48):444-453 (1970)) algorithm which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6.

The term “monoclonal antibody” (mAb) or monoclonal antibody construct as used herein refers to an antibody obtained from a population of substantially homogeneous antibodies, i.e., the individual antibodies comprising the population are identical except for possible naturally occurring mutations and/or post-translation modifications (e.g., isomerizations, amidations) that may be present in minor amounts. Monoclonal antibodies are highly specific, being directed against a single antigenic side or determinant on the antigen, in contrast to conventional (polyclonal) antibody preparations which typically include different antibodies directed against different determinants (or epitopes). The modifier “monoclonal” indicates the character of the antibody as being obtained from a substantially homogeneous population of antibodies, and is not to be construed as requiring production of the antibody by any particular method. A monoclonal antibody may be made by a variety of techniques, including but not limited to generation from a hybridoma, recombinant DNA methods, phage-display and other antibody display methods.

The term “amino acid” or “amino acid residue” typically refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K); methionine (Met or M); phenylalanine (Phe or F); pro line (Pro or P); serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

Amino acid modifications include, for example, deletions from, and/or insertions into, and/or substitutions of, residues within the amino acid sequences of the antibody constructs. Any combination of deletion, insertion, and substitution is made to arrive at the final construct, provided that the final construct possesses the desired characteristics. The amino acid changes also may alter post-translational processes of the antibody constructs, such as changing the number or position of glycosylation sites.

For example, 1, 2, 3, 4, 5, or 6 amino acids may be inserted, substituted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted, substituted or deleted in each of the FRs. Preferably, amino acid sequence insertions into the binding molecule or antibody construct include amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. Corresponding modifications may also performed within the third domain of the antibody construct of the invention. An insertional variant of the antibody construct of the invention includes the fusion to the N-terminus or to the C-terminus of the antibody construct of an enzyme or the fusion to a polypeptide.

The term “polypeptide” as used herein describes a group of molecules, which usually consist of more than 30 amino acids. Polypeptides may further form multimers such as dimers, trimers and higher oligomers, i.e., consisting of more than one polypeptide molecule. Polypeptide molecules forming such dimers, trimers etc. may be identical or non-identical. The corresponding higher order structures of such multimers are, consequently, termed homo- or heterodimers, homo- or heterotrimers etc. An example for a hereteromultimer is an antibody molecule, which, in its naturally occurring form, consists of two identical light polypeptide chains and two identical heavy polypeptide chains. The terms “peptide”, “polypeptide” and “protein” also refer to naturally modified peptides/polypeptides/proteins wherein the modification is effected e.g. by post-translational modifications like glycosylation, acetylation, phosphorylation and the like. A “peptide”, “polypeptide” or “protein” when referred to herein may also be chemically modified such as pegylated. Such modifications are well known in the art and described herein below.

### II. CD19 BINDING MOLECULES INCLUDING CD3- AND CD19-BINDING MOLECULES

Also provided are polynucleotides containing nucleic acids sequences encoding all or a portion of any of the provided binding molecules or antibody constructs, including any of the provided antibodies or antigen-binding fragments or other binding molecules incorporating the same. The provided polynucleotides can be incorporated into constructs, such as deoxyribonucleic acid (DNA) or ribonucleic acid (RNA) constructs, such as those that can be introduced into cells for expression thereof.

Antibody fragments within the scope of the present invention also include F(ab′)2 fragments which may be produced by enzymatic cleavage of an IgG by, for example, pepsin. Fab fragments may be produced by, for example, reduction of F(ab′)2 with dithiothreitol or mercaptoethylamine. A Fab fragment is a VL-CL chain appended to a VH-CH1 chain by a disulfide bridge. A F(ab′)2 fragment is two Fab fragments which, in turn, are appended by two disulfide bridges. The Fab portion of an F(ab′)2 molecule includes a portion of the Fc region between which disulfide bridges are located.

### A. CD19 Antibody Binding Domain

Provided are binding molecules that contain an anti-CD19 antibody binding domain, including full-length antibodies and functional antigen-binding fragments. In some embodiments, the antibodies include antibodies that specifically bind to CD19, e.g., human CD19. Exemplary antibodies are described below. In some embodiments, the antibody, e.g., the anti-CD19 antibody, such as a full-length antibody or antigen-binding antibody fragment, contains a V_{H} sequence and a V_{L} sequence as described. In some embodiments, the anti-CD19 antibody is an antigen-binding antibody fragment that is a single chain fragment, such as a scFv fragment. In some aspects, the scFv comprises a VH region and a VL region. In some embodiments, the anti-CD19 antibody is an antigen-binding antibody fragment that is an Fv fragment containing a V_{H} region and a V_{L} region. In some embodiments, the Fv fragment is a dsFv. In some embodiments, the anti-CD19 antibody is an antigen-binding antibody fragment that is a Fab. In some embodiments, the antibodies provided herein bind to human CD19 with the amino acid sequence set forth in SEQ ID NO: 1, such as is encoded by the nucleotide sequence set forth in SEQ ID NO: 2.

In some embodiments, the anti-CD19 binding region includes one or more copies of an antibody or an antigen-binding fragment thereof selected from the group consisting of a Fab fragment, a F(ab′)2 fragment, an Fv fragment, or an a scFv. In some embodiments, the anti-CD19 binding region is monovalent for binding CD19. In some embodiments, the anti-CD19 binding region is a Fab. In some embodiments, the anti-CD19 binding region is a disulfide stabilized Fv fragment.

Among the provided antibodies, e.g., antigen-binding fragments, are human antibodies. In some embodiments of a provided human anti-CD19 antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a VL region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment.

Among the provided antibodies, e.g., antigen-binding fragments, are human antibodies. In some embodiments of a provided human anti-CD19 antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and contains a VL region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the VH region corresponds to the CDR-H1, the CDR-H2 and/or the CDR-H3. In some embodiments, the portion of the V_{H} region corresponds to the CDR-H1, the CDR-H2 and the CDR-H3. In some embodiments, the portion of the V_{L} region corresponds to the CDR-L1, the CDR-L2 and/or the CDR-L3. In some embodiments, the portion of the VL region corresponds to the CDR-L1, the CDR-L2 and the CDR-L3.

Among the provided antibodies are monoclonal antibodies, including monoclonal antibody fragments.

In some embodiments, the anti-CD19 antibody, e.g., antigen-binding antibody fragment, contains a V_{H} region sequence that contains a CDR-H1, a CDR-H2 and a CDR-H3 as described and contains a V_{L} region sequence that contains a CDR-L1, a CDR-L2 and/or a CDR-L3 as described. Also among the provided antibodies and fragment thereof are those having sequences at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% identical to such a sequence, e.g., any of the CDR-H1, CDR-H2, CDR-H3, CDR-L1, CDR-L2, CDR-L3 sequences of the antibodies described herein. Also among the provided antibodies and fragment thereof are those having sequences at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% identical to such a sequence, e.g., any of the V_{H} and V_{L} sequences, respectively, of the antibodies described herein.

The disclosed binding molecules include anti-CD19 antibodies, including antigen binding fragments, comprising specified paratope resides that interact with the human CD19. In some embodiments, the provided anti-CD19 antibodies, including antibody fragments, comprises a VH region comprising a heavy chain complementarity determining region 1 (CDR-H1), a heavy chain complementarity determining region 2 (CDR-H2), and a heavy chain complementarity determining region 3 (CDR-H3) present in (or contained in) a V_{H} amino acid sequence set forth in any one of SEQ ID NOS: 12, 32, 34, 44, 46, 48, 50, 52, 54, 56, 58, 60, 62, or 88, and a V_{L} region comprising a light chain complementarity determining region 1 (CDR-L1), a light chain complementarity determining region 2 (CDR-L2), and a light chain complementarity determining region 3 (CDR-L3) present in (or contained in) a V_{L} amino acid sequence set forth in any one of SEQ ID NO: 17, 33, 35, 45, 47, 49, 51, 53, 55, 57, 59, 61, 63, or 89.

In some embodiments, the V_{H} region of an antibody or antigen-binding fragment thereof comprises a CDR-H1, a CDR-H2 and a CDR-H3 according to Kabat numbering. In some embodiments, the V_{L} region of an antibody or antigen-binding fragment thereof comprises a CDR-L1, a CDR-L2 and a CDR-L3 according to Kabat numbering.

In some embodiments, the CDR-H1 comprises the amino acid sequence set forth in set forth in any one of SEQ ID NOs: 9, 118, 131, 140, or 146, the CDR-H2 comprises the amino acid sequence set forth in any one of SEQ ID NOs: 10, 119, 132, 141, or 154, and the CDR-H3 comprises the amino acid sequence set forth in any one of SEQ ID NOs: 11, 120, 124, 128, 133, 136, 142, 147, 151, or 155. In some embodiments, the CDR-L1 comprises the amino acid sequence set forth in SEQ ID NOs: 14, 121, 125, 129, 134, 137, 143, 148, 152, or 156, the CDR-L2 comprises the amino acid sequence set forth in any one of SEQ ID NOs: 15, 122, 126, 25, 138, 144, 149, or 157, and the CDR-L3 comprises the amino acid sequence set forth in any one of SEQ ID NOs: 16, 123, 127, 130, 135, 139, 145, 150, 153, or 158.

In some embodiments, the VH region comprises a CDR-H1, a CDR-H2, and a CDR-H3 contained within SEQ ID NO: 32, and the VL region comprises a CDR-L1, a CDR-L2, and a CDR-L3 contained within SEQ ID NO: 33. In some embodiments, the VH region comprises a CDR-H1, a CDR-H2, and a CDR-H3 contained within SEQ ID NO: 34, and the VL region comprises a CDR-L1, a CDR-L2, and a CDR-L3 contained within SEQ ID NO: 35. In some embodiments, the VH region comprises a CDR-H1, a CDR-H2, and a CDR-H3 contained within SEQ ID NO: 44, and the VL region comprises a CDR-L1, a CDR-L2, and a CDR-L3 contained within SEQ ID NO: 45. In some embodiments, the VH region comprises a CDR-H1, a CDR-H2, and a CDR-H3 contained within SEQ ID NO: 46, and the VL region comprises a CDR-L1, a CDR-L2, and a CDR-L3 contained within SEQ ID NO: 47. In some embodiments, the VH region comprises a CDR-H1, a CDR-H2, and a CDR-H3 contained within SEQ ID NO: 48, and the VL region comprises a CDR-L1, a CDR-L2, and a CDR-L3 contained within SEQ ID NO: 49. In some embodiments, the VH region comprises a CDR-H1, a CDR-H2, and a CDR-H3 contained within SEQ ID NO: 50, and the VL region comprises a CDR-L1, a CDR-L2, and a CDR-L3 contained within SEQ ID NO: 51. In some embodiments, the VH region comprises a CDR-H1, a CDR-H2, and a CDR-H3 contained within SEQ ID NO: 52, and the VL region comprises a CDR-L1, a CDR-L2, and a CDR-L3 contained within SEQ ID NO: 53. In some embodiments, the VH region comprises a CDR-H1, a CDR-H2, and a CDR-H3 contained within SEQ ID NO: 54, and the VL region comprises a CDR-L1, a CDR-L2, and a CDR-L3 contained within SEQ ID NO: 55. In some embodiments, the VH region comprises a CDR-H1, a CDR-H2, and a CDR-H3 contained within SEQ ID NO: 56, and the VL region comprises a CDR-L1, a CDR-L2, and a CDR-L3 contained within SEQ ID NO: 57. In some embodiments, the VH region comprises a CDR-H1, a CDR-H2, and a CDR-H3 contained within SEQ ID NO: 58, and the VL region comprises a CDR-L1, a CDR-L2, and a CDR-L3 contained within SEQ ID NO: 59. In some embodiments, the VH region comprises a CDR-H1, a CDR-H2, and a CDR-H3 contained within SEQ ID NO: 60, and the VL region comprises a CDR-L1, a CDR-L2, and a CDR-L3 contained within SEQ ID NO: 61. In some embodiments, the VH region comprises the CDR-H1, the CDR-H2, and the CDR-H3 contained within SEQ ID NO: 62, and the VL region comprises a CDR-L1, a CDR-L2, and a CDR-L3 contained within SEQ ID NO: 63. In some embodiments, the VH region comprises a CDR-H1, a CDR-H2, and a CDR-H3 contained within SEQ ID NO: 12, and the VL region comprises a CDR-L1, a CDR-L2, and a CDR-L3 contained within SEQ ID NO: 17. In some embodiments, the VH region comprises a CDR-H1, a CDR-H2, and a CDR-H3 contained within SEQ ID NO: 88, and the VL region comprises a CDR-L1, a CDR-L2, and a CDR-L3 contained within SEQ ID NO: 89.

In some embodiments, the VH region comprises the CDR-H1, the CDR-H2, and the CDR-H3 comprising the amino acid sequence set forth in SEQ ID NOS: 9, 10, and 11, respectively, and the VL region comprises the CDR-L1, the CDR-L2, and the CDR-L3 comprising the amino acid sequence set forth in SEQ ID NOS: 14, 15, and 16, respectively. In some embodiments, the VH region comprises the CDR-H1, the CDR-H2, and the CDR-H3 comprising the amino acid sequence set forth in SEQ ID NOS: 118, 119, and 120, respectively, and the VL region comprises the CDR-L1, the CDR-L2, and the CDR-L3 comprising the amino acid sequence set forth in SEQ ID NOS: 121, 122, and 123, respectively. In some embodiments, the VH region comprises the CDR-H1, the CDR-H2, and the CDR-H3 comprising the amino acid sequence set forth in SEQ ID NOS:118, 119, and 124, respectively, and the VL region comprises the CDR-L1, the CDR-L2, and the CDR-L3 comprising the amino acid sequence set forth in SEQ ID NOS:125, 126, and 127, respectively. In some embodiments, the VH region comprises the CDR-H1, the CDR-H2, and the CDR-H3 comprising the amino acid sequence set forth in SEQ ID NOS:118, 119, and 128, respectively, and the VL region comprises the CDR-L1, the CDR-L2, and the CDR-L3 comprising the amino acid sequence set forth in SEQ ID NOS: 129, 126, and 130, respectively. In some embodiments, the VH region comprises the CDR-H1, the CDR-H2, and the CDR-H3 comprising the amino acid sequence set forth in SEQ ID NOS: 131, 132, and 133, respectively, and the VL region comprises the CDR-L1, the CDR-L2, and the CDR-L3 comprising the amino acid sequence set forth in SEQ ID NOS: 134, 25, and 135, respectively. In some embodiments, the VH region comprises the CDR-H1, the CDR-H2, and the CDR-H3 comprising the amino acid sequence set forth in SEQ ID NOS: 118, 119, and 136, respectively, and the VL region comprises the CDR-L1, the CDR-L2, and the CDR-L3 comprising the amino acid sequence set forth in SEQ ID NOS:137, 138, and 139, respectively. In some embodiments, the VH region comprises the CDR-H1, the CDR-H2, and the CDR-H3 comprising the amino acid sequence set forth in SEQ ID NOS:140, 141, and 142, respectively, and the VL region comprises the CDR-L1, the CDR-L2, and the CDR-L3 comprising the amino acid sequence set forth in SEQ ID NOS: 143, 144, and 145, respectively. In some embodiments, the VH region comprises the CDR-H1, the CDR-H2, and the CDR-H3 comprising the amino acid sequence set forth in SEQ ID NOS: 146, 119, and 147, respectively, and the VL region comprises the CDR-L1, the CDR-L2, and the CDR-L3 comprising the amino acid sequence set forth in SEQ ID NOS: 148, 149, and 150, respectively. In some embodiments, the VH region comprises the CDR-H1, the CDR-H2, and the CDR-H3 comprising the amino acid sequence set forth in SEQ ID NOS: 118, 119, and 151, respectively, and the VL region comprises the CDR-L1, the CDR-L2, and the CDR-L3 comprising the amino acid sequence set forth in SEQ ID NOS: 152, 126, and 153, respectively. In some embodiments, the VH region comprises the CDR-H1, the CDR-H2, and the CDR-H3 comprising the amino acid sequence set forth in SEQ ID NOS:131, 154, and 155, respectively, and the VL region comprises the CDR-L1, the CDR-L2, and the CDR-L3 comprising the amino acid sequence set forth in SEQ ID NOS: 156, 157, and 158, respectively.

In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 44, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 45. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 46, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 47. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 48, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 49. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 50, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 51. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 52, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 53. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 54, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 55. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 56, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 57. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 58, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 59. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 60, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 61. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 62, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 63. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 12, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 17. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 88, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 89.

In some embodiments, the VH region and the VL region comprise the amino acid sequences set forth in SEQ ID NO: 32 and 33, respectively. In some embodiments, the VH region and the VL region comprise the amino acid sequences set forth in SEQ ID NO: 34 and 35, respectively. In some embodiments, the VH region and the VL region comprise the amino acid sequences set forth in SEQ ID NO: 44 and 45, respectively. In some embodiments, the VH region and the VL region comprise the amino acid sequences set forth in SEQ ID NO: 46 and 47, respectively. In some embodiments, the VH region and the VL region comprise the amino acid sequences set forth in SEQ ID NO: 48 and 49, respectively. In some embodiments, the VH region and the VL region comprise the amino acid sequences set forth in SEQ ID NO: 50 and 51, respectively. In some embodiments, the VH region and the VL region comprise the amino acid sequences set forth in SEQ ID NO: 52 and 53, respectively. In some embodiments, the VH region and the VL region comprise the amino acid sequences set forth in SEQ ID NO: 54 and 55, respectively. In some embodiments, the VH region and the VL region comprise the amino acid sequences set forth in SEQ ID NO: 56 and 57, respectively. In some embodiments, the VH region and the VL region comprise the amino acid sequences set forth in SEQ ID NO: 58 and 59, respectively. In some embodiments, the VH region and the VL region comprise the amino acid sequences set forth in SEQ ID NO: 60 and 61, respectively. In some embodiments, the VH region and the VL region comprise the amino acid sequences set forth in SEQ ID NO: 62 and 63respectively. In some embodiments, the VH region and the VL region of the CD19 binding domain comprises the sequence or are the sequence set forth in SEQ ID NOs: 12 and 17, respectively. In some embodiments, the VH region and the VL region of the CD19 binding domain comprises the sequence or are the sequence set forth in SEQ ID NOs: 88 and 89, respectively.

In some embodiments, the VH region of the CD19 binding domain comprises the CDR-H1, a CDR-H2, and a CDR-H3 comprising the amino acid sequence set forth in SEQ ID NOs: 9, 10, and 11, respectively, and the VL region comprises a CDR-L1, a CDR-L2, and a CDR-L3 comprising the amino acid sequence set forth in SEQ ID NOS: 14, 15, and 16, respectively. In some embodiments the VH region of the CD19 binding domain is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 12, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 17. In some embodiments, the VH region and the VL region of the CD19 binding domain comprises the sequence or are the sequence set forth in SEQ ID NOs: 12 and 17, respectively.

In some embodiments, the VH region of the CD19 binding domain comprises the CDR-H1, a CDR-H2, and a CDR-H3 comprising the amino acid sequence set forth in SEQ ID NOs: 118, 119, and 120, respectively, and the VL region comprises a CDR-L1, a CDR-L2, and a CDR-L3 comprising the amino acid sequence set forth in SEQ ID NOS: 121, 122, 123, respectively. In some embodiments, the VH region of the CD19 binding domain is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 88, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 89. In some embodiments, the VH region and the VL region of the CD19 binding domain comprises the sequence or are the sequence set forth in SEQ ID NOs: 88 and 89, respectively.

In some embodiments, a provided binding molecule is an antibody or an antigen-binding fragment thereof that comprises a VH region and VL region, and their combinations, described above. In some embodiments, the antibody or antigen-binding fragment further includes human immunoglobulin constant regions. In some embodiments, the antibody or antigen-binding fragment is an antigen-binding fragment. In some embodiments, the antigen-binding fragment format retains CD19 binding conferred by the VH and VL regions. In some embodiments, the antigen-binding fragment may be an Fv, scFv, Fab, Fab′, F(ab′)2, or other antigen-binding fragment of antibodies. In some embodiments, the antibody or antigen-binding fragment is a full-length antibody that comprises human immunoglobulin constant regions, including an Fc region.

Antigen-binding fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers, and/or that may not be produced by enzyme digestion of a naturally-occurring intact antibody.

In some embodiments, the antibody is an scFv containing any of the above VH and VL regions. In some embodiments, the scFv includes a linker joining the two antibody VH and VL region. The linker typically is a peptide linker, e.g., a flexible and/or soluble peptide linker, such as one rich in glycine and serine. In some aspects, the linker that is rich in glycine and serine (and/or threonine) includes at least 80%, 85%, 90%, 91%, 92%, 93%, 94%, 95%, 96%, 97%, 98%, or 99% glycine, serine, and/or threonine amino acid(s). In some embodiments, the linker includes at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 160) or GGGS (3GS; SEQ ID NO: 217), such as between 2, 3, 4 and 5 repeats of such a sequence. Exemplary linkers include those having the sequence set forth in SEQ ID NO: 162 (GGGGSGGGGSGGGGS). Exemplary linkers further include those having the sequence set forth in SEQ ID NO: 161 (GGGGSGGGGS). Exemplary linkers further include those having the sequence set forth in SEQ ID NO: 218 (GSTSGSGKPGSGEGSTKG). Exemplary linkers further include those having or consisting of the sequence set forth in SEQ ID NO: 219 (SRGGGGSGGGGSGGGGSLEMA). An exemplary linker includes those having or consisting of the sequence set forth in SEQ ID NO: 220 (GSRGGGGSGGGGSGGGGSLEMA). In some embodiments, the VH region may be amino terminal to the VL region. In some embodiments, the VH region may be carboxy terminal to the VL region. In particular embodiments, the fragment, e.g., scFv, may include a VH region or portion thereof, followed by the linker, followed by a VL region or portion thereof. In other embodiments, the fragment, e.g., the scFv, may include the VL region or portion thereof, followed by the linker, followed by the VH region or portion thereof.

In some embodiments, the scFv is a disulfide-stabilized Fv (ds-scFv) in which the VH and VL contain a first and second engineered cysteine residue, respectively, that form a disulfide bond. In some embodiments, the VH region comprises a cysteine residue at framework region 2 position 44 (Kabat numbering) and the VL region comprises a cysteine residue at framework region 4 position 100 (Kabat numbering).

In some embodiments, the CD19 binding domain is an scFv comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 245. In some embodiments, the scFv comprises the amino acid sequence of SEQ ID NO: 245. In some embodiments, the CD19 binding domain is an scFv comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 248. In some embodiments, the scFv comprises the amino acid sequence of SEQ ID NO: 248. In some embodiments, the CD19 binding domain is an scFv comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 249. In some embodiments, the scFv comprises the amino acid sequence of SEQ ID NO: 249. In some embodiments, the CD19 binding domain is an scFv comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 252. In some embodiments, the scFv comprises the amino acid sequence of SEQ ID NO: 252.

In some embodiments, the antibody is an antigen-binding fragment comprising the VH and VL regions that further comprises at least a portion of an immunoglobulin constant region of the heavy chain and at least a portion of an immunoglobulin constant region of the light chain. In some embodiments, the CD19 binding domain is an anti-CD19 antibody that is a Fab containing any of the above VH and VL regions. In some embodiments, the Fab contains a heavy chain comprising any one of the above VH regions and an immunoglobulin constant heavy chain domain 1 (CH1 domain) and a light chain comprising any one of the above VL regions and an immunoglobulin constant light chain (CL).

In some embodiments, the CH1 is from an IgG1 antibody. In some embodiments, the CH1 is set forth by the sequence in SEQ ID NO: 221 or a sequence having at least at or about 85%, 86%, 87%, 88%, 89%, 90%, 91%, 92%, 93%, 94%, 95%, 96%, 97%, 98%, 99% identity to SEQ ID NO:221. In some embodiments, the CH1 is set forth by the sequence in SEQ ID NO: 223 or a sequence having at least at or about 85%, 86%, 87%, 88%, 89%, 90%, 91%, 92%, 93%, 94%, 95%, 96%, 97%, 98% or 99% identity to SEQ ID NO:223. In some embodiments, the CH1 is from an IgG1 antibody. In some embodiments, the CH1 is set forth by the sequence in SEQ ID NO: 169 or a sequence having at least at or about 85%, 86%, 87%, 88%, 89%, 90%, 91%, 92%, 93%, 94%, 95%, 96%, 97%, 98% or 99% identity to SEQ ID NO:169.

In some embodiments, the CH1 is from an IgG2 antibody. In some embodiments, the CH1 is set forth by the sequence in SEQ ID NO: 170 or a sequence having at least at or about 85%, 86%, 87%, 88%, 89%, 90%, 91%, 92%, 93%, 94%, 95%, 96%, 97%, 98% or 99% identity to SEQ ID NO:170.

In some embodiments, the CL is a kappa constant domain. In some embodiments, the constant light chain (CL) is set forth by the sequence in SEQ ID NO: 171 or a sequence having at least at or about 85%, 86%, 87%, 88%, 89%, 90%, 91%, 92%, 93%, 94%, 95%, 96%, 97%, 98% or 99% identity to SEQ ID NO:171. In some embodiments, the constant light chain (CL) is set forth by the sequence in SEQ ID NO: 225 or a sequence having at least at or about 85%, 86%, 87%, 88%, 89%, 90%, 91%, 92%, 93%, 94%, 95%, 96%, 97%, 98% or 99% identity to SEQ ID NO: 225.

In some embodiments, the CL is a lambda constant domain. In some embodiments, the constant light (CL) is set forth by the sequence in SEQ ID NO: 224 or a sequence having at least at or about 85%, 86%, 87%, 88%, 89%, 90%, 91%, 92%, 93%, 94%, 95%, 96%, 97%, 98% or 99% identity to SEQ ID NO: 224.

In some embodiments, the CD19 binding domain is an anti-CD19 antibody that is a Fab composed of a heavy chain comprising any one of the above VH regions and an immunoglobulin constant heavy chain domain 1 (CH1 domain) set forth in SEQ ID NO: 169, and a light chain comprising any one of the above VL regions and an immunoglobulin constant light chain (CL) set forth in SEQ ID NO: 171.

In some such embodiments, the antibody is a full-length antibody that also contains a heavy chain and light chain constant region. In some embodiments, the anti-CD19 antibody, or an antigen binding fragment thereof, is a human IgG. In some embodiments, the antibody comprises a constant region from an IgG1 or IgG2. In some embodiments, the constant region is a human IgG1 heavy chain constant region. In some embodiments, the antibody includes at least a portion of a hinge region or a variant thereof. In some embodiments, the antibody includes a CH2 and/or CH3 domain, such as an Fc region. In some embodiments, the Fc region is an Fc region of a human IgG, such as an IgG1 or IgG4. In some embodiments, the heavy chain constant portion is derived from an IgG1 immunoglobulin constant chain. In some embodiments, the light chain CL is from a kappa or lambda light chain sequence.

In full-length antibody embodiments, the antibody comprises two heavy chains and two light chains (H2L2), wherein each heavy chain comprises a VH region and a heavy-chain constant region and each light chain comprises a VL region and a light-chain constant region, and the heavy chain may include an Fc region comprising at least part of the hinge, CH2, and CH3 domains. Among the provided antibodies are full-length antibodies containing a heavy chain with any one of the VH regions provided herein combined with a human heavy chain constant region; and a light chain with any one of the VL regions provided herein combined with a human light chain constant region.

In some embodiments, the constant region of the heavy chain is a human IgG1 heavy chain constant region. In some embodiments, the heavy chain constant region is set forth in SEQ ID NO: 226 or a sequence that has at least 85%, 86%, 87%, 88%, 89%, 90%, 91%, 92%, 93%, 94%, 95%, 96%, 97%, 98%, or 99% sequence identity to SEQ ID NO: 226. In some embodiments, the heavy chain constant region is set forth in SEQ ID NO: 226. In some embodiments, the heavy chain constant region is set forth in SEQ ID NO: 227 or a sequence that has at least 85%, 86%, 87%, 88%, 89%, 90%, 91%, 92%, 93%, 94%, 95%, 96%, 97%, 98%, or 99% sequence identity to SEQ ID NO: 227. In some embodiments, the heavy chain constant region is set forth in SEQ ID NO: 227.

In some embodiments, the constant light chain is a λ (lambda) light chain. In some embodiments, the constant light (CL) is set forth by the sequence in SEQ ID NO: 224 or a sequence having at least at or about 85%, 86%, 87%, 88%, 89%, 90%, 91%, 92%, 93%, 94%, 95%, 96%, 97%, 98% or 99% identity to SEQ ID NO:224.

In some embodiments, the constant light chain is a κ (kappa) light chain. In some embodiments, the constant light chain (CL) is set forth by the sequence in SEQ ID NO: 171 or a sequence having at least at or about 85%, 86%, 87%, 88%, 89%, 90%, 91%, 92%, 93%, 94%, 95%, 96%, 97%, 98% or 99% identity to SEQ ID NO:171.

In some embodiments, the constant light chain is a κ (kappa) light chain. In some embodiments, the constant light chain (CL) is set forth by the sequence in SEQ ID NO: 225 or a sequence having at least at or about 85%, 86%, 87%, 88%, 89%, 90%, 91%, 92%, 93%, 94%, 95%, 96%, 97%, 98% or 99% identity to SEQ ID NO: 225.

In some embodiments, the heavy chain constant region is mutated or modified relative to a wild-type immunoglobulin constant region (e.g., a human IgG constant region) to provide a modified (or variant) Fc region, when an Fc region or portion thereof is present. In some embodiments, the modified constant region comprises a modified Fc region (e.g., a human IgG1, IgG2, IgG3, or IgG4 Fc region) comprising one or more amino acid modifications (e.g., substitutions and/or deletions) at one or more amino acid positions. In some embodiments, the Fc region comprises a hinge region, CH2, and CH3.

In some cases, the mutations include one or more amino acid substitutions in the heavy chain constant region, such as the heavy chain constant region of IgG1, to reduce effector activity of the heavy chain constant region. In some embodiments, the Fc region is mutated or modified to reduce or eliminate one or more Fc effector functions (e.g., Fcγ receptor binding and/or C1q binding). In some embodiments, such mutation or modification substantially retains FcRn binding. Exemplary mutations in an Fc that can be included to reduce or eliminate one or more Fc effector functions, optionally while retaining FcRn binding are described in Section II.B.2. Any other mutation (e.g., substitution) described in Section II.B.2 may also be included an Fc constant region sequence of a provided antibody.

In some embodiments, a CD19 binding domain is a full length antibody that is capable of forming a dimer via Fc-mediated association. In some embodiments, the Fc region comprises a first Fc-containing polypeptide chain and a second Fc-containing polypeptide chain that associate to form a homodimer comprising two identical Fc-containing polypeptide chains. In some embodiments, dimerization is mediated by interactions within one or more constant-region domains of the Fc region (e.g., hinge, CH2, and/or CH3) and, where present, inter-chain disulfide bonds in the hinge. In some embodiments, the Fc region is formed by Fc domains that are mutated or modified to promote heterodimerization in which different polypeptides can be dimerized to yield a heterodimer. Thus, in some embodiments, the dimer is a heterodimer in which two polypeptide chains of the antibody are different. Exemplary modifications to promote heterodimerization are known, including any as described below in Section II.B.2.

In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 254; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 255. In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 256; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 257. In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 258; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 259. In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 260; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 261. In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 262; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 263. In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 264; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 265. In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 266; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 267. In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 268; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 269. In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 270; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 271. In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 272; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 273. In some embodiments, the anti-CD19 antibody, or an antigen binding fragment thereof comprises: (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 274; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 275.

In some embodiments, the anti-CD19 antibody, or an antigen binding fragment thereof comprises: (i) a heavy chain comprising the amino acid sequence of SEQ ID NO: 254; and (ii) a light chain comprising the amino acid sequence of SEQ ID NO: 255. In some embodiments, the anti-CD19 antibody, or an antigen binding fragment thereof comprises: (i) a heavy chain comprising the amino acid sequence of SEQ ID NO: 256; and (ii) a light chain comprising the amino acid sequence of SEQ ID NO: 257. In some embodiments, the anti-CD19 antibody, or an antigen binding fragment thereof comprises: (i) a heavy chain comprising the amino acid sequence of SEQ ID NO: 258; and (ii) a light chain comprising the amino acid sequence of SEQ ID NO: 259. In some embodiments, the anti-CD19 antibody, or an antigen binding fragment thereof comprises: (i) a heavy chain comprising the amino acid sequence of SEQ ID NO: 260; and (ii) a light chain comprising the amino acid sequence of SEQ ID NO: 261. In some embodiments, the anti-CD19 antibody, or an antigen binding fragment thereof comprises: (i) a heavy chain comprising the amino acid sequence of SEQ ID NO: 262; and (ii) a light chain comprising the amino acid sequence of SEQ ID NO: 263. In some embodiments, the anti-CD19 antibody, or an antigen binding fragment thereof comprises: (i) a heavy chain comprising the amino acid sequence of SEQ ID NO: 264; and (ii) a light chain comprising the amino acid sequence of SEQ ID NO: 265. In some embodiments, the anti-CD19 antibody, or an antigen binding fragment thereof comprises: (i) a heavy chain comprising the amino acid sequence of SEQ ID NO: 266; and (ii) a light chain comprising the amino acid sequence of SEQ ID NO: 267. In some embodiments, the anti-CD19 antibody, or an antigen binding fragment thereof comprises: (i) a heavy chain comprising the amino acid sequence of SEQ ID NO: 268; and (ii) a light chain comprising the amino acid sequence of SEQ ID NO: 269. In some embodiments, the anti-CD19 antibody, or an antigen binding fragment thereof comprises: (i) a heavy chain comprising the amino acid sequence of SEQ ID NO: 270; and (ii) a light chain comprising the amino acid sequence of SEQ ID NO: 271. In some embodiments, the anti-CD19 antibody, or an antigen binding fragment thereof comprises: (i) a heavy chain comprising the amino acid sequence of SEQ ID NO: 272; and (ii) a light chain comprising the amino acid sequence of SEQ ID NO: 273. In some embodiments, the anti-CD19 antibody, or an antigen binding fragment thereof comprises: (i) a heavy chain comprising the amino acid sequence of SEQ ID NO: 274; and (ii) a light chain comprising the amino acid sequence of SEQ ID NO: 275.

Also provided are methods of making the CD19 binding molecules, including anti-CD19 antibodies (including antigen-binding fragments) or a binding molecule incorporating such antibodies or fragments. For recombinant production of the binding molecule, such as an anti-CD19 antibody, a polynucleotide sequence or a polynucleotide encoding an antibody, e.g., as described above, may be isolated and inserted into one or more vectors for further cloning and/or expression in a host cell. Such polynucleotide sequences may be readily isolated and sequenced using conventional procedures (e.g., by using oligonucleotide probes that are capable of binding specifically to genes encoding the heavy and light chains of the antibody). In some embodiments, a method of making the CD19 binding molecules, such as anti-CD19 antibody, is provided, wherein the method comprises culturing a host cell comprising a polynucleotide sequence encoding the binding molecule, as provided above, under conditions suitable for expression of the binding molecule, and optionally recovering the binding molecule from the host cell (or host cell culture medium).

Exemplary eukaryotic cells that may be used to express polypeptides include, but are not limited to, COS cells, including COS 7 cells; 293 cells, including 293-6E cells; CHO cells, including CHO-S, DG44. Lecl3 CHO cells, and FUT8 CHO cells; PER.C6® cells; and NSO cells. In some embodiments, the antibody heavy chains and/or light chains (e.g., VH region and/or VL region) may be expressed in yeast. See, e.g., U.S. Publication No. US 2006/0270045 A1. In some embodiments, a particular eukaryotic host cell is selected based on its ability to make desired post-translational modifications to the heavy chains and/or light chains (e.g., VH region and/or VL region). For example, in some embodiments, CHO cells produce polypeptides that have a higher level of sialylation than the same polypeptide produced in 293 cells.

In some embodiments, the antibody or antigen-binding fragment provided herein is produced in a cell-free system. Exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

In some of any of such embodiments, the anti-CD19 antibody or antigen-binding fragment thereof comprises at least one post-translational modification of the amino acid sequence. Post-translational modifications can include, e.g., glycosylation, phosphorylation, citrullination, isomerization, ubiquitination, acetylation, hydroxylation, methylation, AMPylation, prenylation, deamidation, eliminylation, carbamylation and carbamoylation. In some embodiments, the post-translational modification is the modification of an amino acid side chain, such as conversion of an N-terminal amino acid (e.g., glutamate or glutamine) to pyroglutamate. In some such embodiments, this modification occurs at the N-terminus of the heavy chain. In some embodiments, the post-translational modification includes a post-translational modification of a heavy chain N-terminal glutamine (Q) to a pyroglutamate. In some embodiments, in a composition of an antibody or antigen-binding fragment thereof as disclosed herein, such a post-translational modification to pyroglutamate (e.g., at the N-terminus of the heavy chain) is a dominant post-translational modification; in some such embodiments, the antibody or antigen binding fragment thereof in which an N-terminal pyroglutamate is present represents greater than 50%, greater than 60%, greater than 70%, greater than 80%, greater than 90%, or greater than 95% (w/w) of the antibody or antigen binding fragment thereof in the composition.

In some embodiments, the post-translational modification is the cleavage of one or more, particularly one or two, amino acids from the C-terminus of the heavy chain. For instance, an antibody produced by expression of a specific nucleic acid molecule encoding a full-length heavy chain (e.g., by expression in a host cell) may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, an anti-CD19 antibody may include a heavy chain constant domain with both a C-terminal glycine and lysine, without the C-terminal lysine, or without both the C-terminal glycine and lysine. In some embodiments, in a composition of an antibody or antigen-binding fragment thereof as disclosed herein, glycine cleavage is not a dominant post-translational modification; in some such embodiments, the antibody or antigen binding fragment thereof in which the C-terminal lysine is cleaved from the heavy chain constant domain represents less than 10%, less than 5%, or less than 4% (w/w) of the antibody or antigen binding fragment thereof in the composition.

### B. Bispecific Binding Molecules

Among the binding molecules are CD3- and CD19-bispecific antibodies (including antigen-binding fragments). In some embodiments, the provided CD3- and CD19-binding molecules that contain an antibody binding domain that binds CD19 (also called anti-CD19 antibodies) and an antibody binding domain that binds CD3 (also called anti-CD3 antibodies). Such bispecific binding molecules are also referred to as T cell engagers (TCEs) since they incorporate an anti-CD3 antibody or antigen-binding fragment, in addition to the anti-CD19 antibody or antigen-binding fragment. Such antibody binding domains specifically bind to CD3 epsilon (CD3F) proteins, such as a human CD3 ε protein and CD19 proteins, such as human CD19. In some embodiments, the antibodies or antigen-binding fragments include a heavy chain variable region (V_{H}) and a light chain variable region (V_{L}), including full-length antibodies as well as functional antigen-binding fragments such as Fabs or single chain Fv fragments (scFvs).

Among the provided anti-CD3 and anti-CD19 antibodies are human antibodies, or antibodies that are modified from or variant of human antibodies. The antibodies include isolated antibodies.

In some embodiments, the anti-CD19 antibody or antigen-binding fragment is any as described in Section II.A. In some embodiments, the anti-CD3 antibody or antigen-binding fragment is any described below in Section II.B.1.

In some embodiments, the bipecific antibody is composed of at least one anti-CD19 antibody or antigen-binding fragment (e.g., anti-CD19 scFv or Fab) directed against CD19, and at least one anti-CD3 antibody or antigen-binding fragment (e.g. anti-CD3 Fab or scFv) directed against CD3. In any of such embodiments, the binding molecule is a bispecific antigen binding molecule, such as a bispecific antibody, that has binding specificity for both CD3 and CD19.

In some embodiments, the anti-CD3 antibody (including antigen-binding fragment) incorporated in the bispecific molecule is able to activate T cells via engagement of CD3, such as CD3ε, on the T cells. The anti-CD3 antibody exhibits activity to agonize, stimulate, activate, and/or otherwise augment CD3-mediated T cell activation. Biological activities of CD3 include, for example, T cell activation and other signaling through interaction between CD3 and the antigen-binding subunits of the T-Cell Receptor (TCR). Any of a variety of methods are known for measuring T cell activation. In some embodiments, T cell activation can be assessed by determining the ability of a provided bispecific antibody to stimulate cytokine production, proliferation or cytotoxic activity in T cell cultures. In some embodiments, the activity is present in co-cultures of T cells with antigen-expressing target cells but not present in the absence of antigen-expressing cells. In some embodiments, the bispecific antibodies elicit potent T cell activation or T cell killing when also engaged by or bound to the target (second) antigen, such as target antigen-expressing target cells (e.g. CD19-expresssing tumor cells).

In some embodiments, the binding molecule containing a CD3 binding domain provides for a T cell engaging therapy. The T cell-engaging therapy is a bispecific antibody containing at least one antigen-binding domain binding to an activating component of the T cell such as CD3 and at least one antigen-binding domain binding to CD19 that is present on a target cell (e.g., B cell) desired to be targeted. In some embodiments, the simultaneous or near simultaneous binding of such an antibody to both of its targets can result in a temporary interaction between the target cell and T cell, thereby resulting in activation, e.g. cytotoxic activity, of the T cell and subsequent lysis of the target cell.

The provided bispecific antibodies provide a targeted antibody therapy for delivering cytotoxic T cells specifically to the target cells expressing CD19, including autoreactive B cells. Hence, in some aspects the provided bispecific antibodies may also be called T cell engagers (TCE). In particular embodiments, the provided bispecific antibodies can be used in therapeutic settings in which specific targeting and T cell-mediated killing of cells that express CD19 is desired. In particular embodiments, the provided bispecific antibodies can be used in therapeutic settings in which specific targeting and T cell-mediated killing of autoreactive B cells is desired.

In some embodiments, the bispecific antibody is capable of binding CD19 on B cells (or on CD19-expressing tumor cells) and CD3 on T cells. In some embodiments, the bispecific antibody results in B cell and/or plasma blast depletion of greater than 90% in the lymph node, bone marrow, and/or blood as determined by an in vitro cell depletion assay.

In some embodiments, biological activity or functional activity of a provided bispecific antibody, such as cytotoxic activity, can be measured using any of a number of known methods. The activity can be assessed or determined either in vitro or in vivo. In some embodiments, activity can be assessed once the bispecific antibody is administered to the subject (e.g., human). Parameters to assess include specific binding of the bispecific antibody to a T cell (e.g. natural T cell) or target antigen-expressing cell, e.g., in vivo, e.g., by imaging, or ex vivo, e.g., by ELISA or flow cytometry. In certain embodiments, the ability of the bispecific antibody to destroy target cells can be measured using any suitable known methods, such as cytotoxicity assays described in, for example, Kochenderfer et al., J. Immunotherapy, 32(7): 689-702 (2009), and Herman et al. J. Immunological Methods, 285(1): 25-40 (2004). In certain embodiments, the biological activity of the bispecific antibody also can be measured by assaying expression and/or secretion of certain cytokines, such as interlekukin-2 (IL-2), interferon-gamma (IFNγ), interleukin-1 beta (IL-1β), interleukin-4 (IL-4), TNF-alpha (TNFα), interleukin-6 (IL-6), interleukin-10 (IL-10), interleukin-12 (IL-12), interleukin-13 (IL-13), granulocyte-macrophage colony-stimulating factor (GM-CSF), CD107a, and/or TGF-beta (TGFβ). Assays to measure cytokines are well known, and include but are not limited to, ELISA, intracellular cytokine staining, cytometric bead array, RT-PCR, ELISPOT, flow cytometry and bio-assays in which cells responsive to the relevant cytokine are tested for responsiveness (e.g. proliferation) in the presence of a test sample. In some aspects the biological activity can be measured using an animal model of the disease or condition, such as a tumor xenograft model, and assessing the reduction in tumor burden or load and/or survival. In some aspects the biological activity is measured by assessing clinical outcome, such as reduction in tumor burden or load.

A bispecific antibody provided herein is not limited to any particular bispecific format or method of producing it. Bispecific antibodies can be prepared as full length antibodies or antibody fragments. Among the multispecific antibodies are multispecific single-chain antibodies, e.g., diabodies, triabodies, and tetrabodies, tandem di-scFvs, and tandem tri-scFvs. In some embodiments, the bispecific antibody is formatted as a bivalent antibody. In some embodiments, the bispecific antibody is formatted as a trivalent antibody.

In some embodiments, each antigen-binding domain, including the anti-CD3 binding domain and the anti-CD19 binding domain, comprise an antibody or an antigen-binding fragment. In some embodiments, the bispecific antibody contains one CD3 binding domain and one CD19 binding domain.

In some embodiments, the CD3 binding domain includes an antibody or an antigen-binding fragment thereof selected from the group consisting of a Fab fragment, a F(ab′)2 fragment, an Fv fragment, an scFv such as a dsFv, a scAb, a dAb, a single domain heavy chain antibody, and a single domain light chain antibody. In some embodiments, the CD3 binding domain is a Fab. In some embodiments, the CD3 binding domain is an scFv, such as a dsFv. In some embodiments, the CD3 binding domain can include any of the antibodies or antigen-binding fragments as described in Section II.B.1.

In some embodiments, the CD19 binding domain includes an antibody or an antigen-binding fragment thereof selected from the group consisting of a Fab fragment, a F(ab′)2 fragment, an Fv fragment, an scFv such as a dsFv, a scAb, a dAb, a single domain heavy chain antibody, and a single domain light chain antibody. In some embodiments, the CD19 binding domain is a Fab. In some embodiments, the CD19 binding domain is an scFv, such as a dsFv. In some embodiments, the CD19 binding domain can include any of the antibodies or antigen-binding fragments as described in Section II.A.

The at least two binding domains and the variable domains (VH/VL) of the antibody construct of the present invention may or may not comprise peptide linkers (spacer peptides). The term “peptide linker” comprises in accordance with the present invention an amino acid sequence by which the amino acid sequences of one (variable and/or binding) domain and another (variable and/or binding) domain of the antibody construct of the invention are linked with each other. The peptide linkers can also be used to fuse the third domain to the other domains of the antibody construct of the invention. An essential technical feature of such peptide linker is that it does not comprise any polymerization activity. Among the suitable peptide linkers are those described in U.S. Pat. Nos. 4,751,180 and 4,935,233 or WO 88/09344. The peptide linkers can also be used to attach other domains or modules or regions (such as half-life extending domains) to the antibody construct of the invention.

Methods of producing bi-specific T cell engagers are known, including fusion of two different hybridomas (Milstein and Cuello, Nature 1983; 305:537-540), chemical tethering though heterobifunctional cross linkers (Staerz et al. Nature 1985; 314:628-631), manufacturing of scFv-based agents of multivalency and multispecificity by varying the linker length (e.g. U.S. Pat. Nos. 5,844,094, 5,837,242 and WO 98/44001), diabodies including Dual-Affinity Re-Targeting (DART®) (see, e.g., Moore et al., 2011, Blood 117:4542-51; Veri et al, 2010, Arthritis Rheum 62: 1933-43), and tetravalent and bispecific antibody-like proteins known as DVD-Igs which are engineered from two monoclonal antibodies (Wu, C. et al., Nature Biotechnology, 25, p 1290-1297, 2007). Exemplary bispecific formats are known, see e.g. Brinkmann et al. MAbs 2017, 9:182-212.

For instance, among such exemplary bispecific antibodies are bispecific T cell engager (BiTE) molecules, which contain tandem scFv molecules fused by a flexible linker (see e.g. Nagorsen and Bauerle, Exp Cell Res 317, 1255-1260 (2011); tandem scFv molecules fused to each other via, e.g. a flexible linker, and that further contain an Fc domain composed of a first and a second subunit capable of stable association (WO2013026837); diabodies and derivatives thereof, including tandem diabodies (Holliger et al, Prot Eng 9, 299-305 (1996); Kipriyanov et al, J Mol Biol 293, 41-66 (1999)); dual affinity retargeting (DART) molecules that can include the diabody format with a C-terminal disulfide bridge; or triomabs that include whole hybrid mouse/rat IgG molecules (Seimetz et al, Cancer Treat Rev 36, 458-467 (2010); and trivalent bispecific antibodies (e.g. Mayer et al. 2015 Int. J Mol Sci, 16:27497-27507 and U.S. patent publication No. US20200062826).

Examples of bispecific antibody molecules formats which may be used include (i) a single antibody that has two arms comprising different antigen-binding regions; (ii) a single chain antibody that has specificity to two different epitopes, e.g., via two scFvs linked in tandem by an extra peptide linker; (iii) a dual-variable-domain antibody (DVD-Ig), where each light chain and heavy chain contains two variable domains in tandem through a short peptide linkage (Wu et al., Generation and Characterization of a Dual Variable Domain Immunoglobulin (DVD-Ig™) Molecule, In: Antibody Engineering, Springer Berlin Heidelberg (2010)); (iv) a chemically-linked bispecific (Fab′)2 fragment; (v) a Tandab, which is a fusion of two single chain diabodies resulting in a tetravalent bispecific antibody that has two binding sites for each of the target antigens; (vi) a flexibody, which is a combination of scFvs with a diabody resulting in a multivalent molecule; (vii) a so-called “dock and lock” molecule, based on the “dimerization and docking domain” in Protein Kinase A, which, when applied to Fabs, can yield a trivalent bispecific binding protein consisting of two identical Fab fragments linked to a different Fab fragment; (viii) a so-called Scorpion molecule, comprising, e.g., two scFvs fused to both termini of a human Fab-arm; and (ix) a diabody.

In some embodiments, bispecific antibodies are bivalent bispecific antibodies. In some embodiments, bivalent bispecific antibody include two antigen binding sites, namely one antigen binding sites for CD19 and one antigen binding site for CD3. In some embodiments, a bivalent bispecific antibody is monovalent for binding to CD19 and monovalent for binding to CD3.

In some embodiments, the bispecific antibody provided herein is composed of a CD3 binding domain that is a fragment of an antibody that specifically binds to CD3, a CD19 binding domain with a fragment of an antibody that specifically binds to CD19, and a Fc region. In some embodiments, the CD3 binding domain and the CD19 binding domain are linked via their C-termini to the hinge region of the Fc region. In some embodiments, the Fc region acts to multimerize the two heavy chain polypeptides of the binding molecule. Hence, the Fc region is a multimerization domain. In some embodiments, the Fc region is a heterodimeric Fc region that facilitates formation of a heterodimer containing two different heavy chain polypeptides in the bispecific binding molecule.

In some embodiments, the bispecific antibody provided herein is composed of a CD3 binding domain that is a Fab fragment for specifically binding to CD3 (further named also as “CD3-Fab”), a CD19 binding domain that is an scFv fragment for specifically binding CD19 (further named also as “CD19-scFv”, such as “CD19-dsFv”), and a Fc region. In some embodiments, the CD3 binding domain and the CD19 binding domain are linked via their C-termini to the hinge region of the Fc region.

In some embodiments, the bispecific antibody provided herein is composed of a CD3 binding domain that is a scFv fragment, such as a dsFv, specifically binding to CD3 (further named also as “CD3-scFv”, such as “CD3-dsFv”), a CD19 binding domain with an antibody means for binding to the CD19 (further named also as “CD19-scFv”, such as “CD19-dsFv”), and a Fc region. In some embodiments, the CD3 binding domain and the CD19 binding domain are linked via their C-termini to the hinge region of the Fc region.

In some of any of such embodiments, the bispecific binding molecule, including the anti-CD19 antibody or antigen-binding fragment thereof and/or the anti-CD3 antibody or antigen-binding fragment thereof, comprises at least one post-translational modification of the amino acid sequence. Post-translational modifications can include, e.g., glycosylation, phosphorylation, citrullination, isomerization, ubiquitination, acetylation, hydroxylation, methylation, AMPylation, prenylation, deamidation, eliminylation, carbamylation and carbamoylation. In some embodiments, the post-translational modification is the modification of an amino acid side chain, such as conversion of an N-terminal amino acid (e.g., glutamate or glutamine) to pyroglutamate. In some such embodiments, this modification occurs at the N-terminus of the heavy chain. In some embodiments, the post-translational modification includes a post-translational modification of a heavy chain N-terminal glutamine (Q) to a pyroglutamate. In some embodiments, in a composition of an antibody or antigen-binding fragment thereof as disclosed herein, such a post-translational modification to pyroglutamate (e.g., at the N-terminus of the heavy chain) is a dominant post-translational modification; in some such embodiments, the antibody or antigen binding fragment thereof in which an N-terminal pyroglutamate is present represents greater than 50%, greater than 60%, greater than 70%, greater than 80%, greater than 90%, or greater than 95% (w/w) of the antibody or antigen binding fragment thereof in the composition.

In some embodiments, the post-translational modification is the cleavage of one or more, particularly one or two, amino acids from the C-terminus of the heavy chain. For instance, an antibody produced by expression of a specific nucleic acid molecule encoding a full-length heavy chain (e.g., by expression in a host cell) may include the full-length heavy chain, or it may include a cleaved variant of the full-length heavy chain. This may be the case where the final two C-terminal amino acids of the heavy chain are glycine and lysine, respectively. Therefore, the C-terminal lysine, or the C-terminal glycine and lysine, of the Fc region may or may not be present. Thus, an antibody may include a heavy chain constant domain with both a C-terminal glycine and lysine, without the C-terminal lysine, or without both the C-terminal glycine and lysine. In some embodiments, in a composition of an antibody or antigen-binding fragment thereof as disclosed herein, glycine cleavage is not a dominant post-translational modification; in some such embodiments, the antibody or antigen binding fragment thereof in which the C-terminal lysine is cleaved from the heavy chain constant domain represents less than 10%, less than 5%, or less than 4% (w/w) of the antibody or antigen binding fragment thereof in the composition.

### 1. CD3 Antibody Binding Domain

In some embodiments, the provided binding molecules contain a CD3 antibody binding domain, including full-length antibodies and functional antigen-binding fragments. In some embodiments, the antibodies include antibodies that specifically bind to CD3 ε e.g., human CD3 ε. Exemplary antibodies are described below. In some embodiments, the antibody, e.g., the anti-CD3 antibody, such as a full-length antibody or antigen-binding antibody fragment, contains a heavy chain variable region (V_{H}) sequence and a light chain variable region (V_{L}) sequence as described. In some embodiments, the anti-CD3 antibody is an antigen-binding antibody fragment that is a single chain fragment, such as a single chain Fv (scFv) fragment. In some aspects, the scFv comprises a V_{H} region and a V_{L} region. In some embodiments, the anti-CD3 antibody is an antigen-binding antibody fragment that is an scFv fragment containing a V_{H} region and a V_{L} region. In some embodiments, the scFv fragment is a disulfide-stabilized Fv (ds-scFv). In some embodiments, the anti-CD3 antibody is an antigen-binding antibody fragment that is a Fab. In some embodiments, the antibodies provided herein bind to human CD3 ε set forth in SEQ ID NO:5 (e.g, UniProt P07766 or NP_000724.1).

The anti-CD3 binding region of the disclosure agonize, stimulate, activate, and/or otherwise augment CD3-mediated T cell activation. Biological activities of CD3 include, for example, T cell activation and other signaling through interaction between CD3 and the antigen-binding subunits of the T-Cell Receptor (TCR). For example, the anti-CD3 binding domains of the disclosure completely or partially activate T cells via engagement of CD3 (e.g., CD3ε) on T cells by partially or completely modulating, e.g., agonizing, stimulating, activating or otherwise augmenting CD3-mediated T cell activation.

In some embodiments, the anti-CD3 binding region includes one or more copies of an antibody or an antigen-binding fragment thereof selected from the group consisting of a Fab fragment, a F(ab′)2 fragment, an Fv fragment, or an scFv. In some embodiments, the anti-CD3 binding region is monovalent for binding CD3. In some embodiments, the anti-CD3 binding region is a Fab. In some embodiments, the anti-CD3 binding region is a disulfide stabilized Fv fragment.

Among the provided antibodies, e.g., antigen-binding fragments, are human antibodies. In some embodiments of a provided human anti-CD3 antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and/or contains a VL region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment.

Among the provided antibodies, e.g., antigen-binding fragments, are human antibodies. In some embodiments of a provided human anti-CD3 antibody, e.g., antigen-binding fragments, the human antibody contains a V_{H} region that comprises a portion having at least 95%, 96%, 97%, 98%, 99%, or 100% sequence identity to an amino acid sequence encoded by a germline nucleotide human heavy chain V segment, a portion having at least 95%, 96%, 97%, 98%, 99%, or 100% sequence identity to an amino acid sequence encoded by a germline nucleotide human heavy chain D segment, and/or a portion having at least 95%, 96%, 97%, 98%, 99%, or 100% sequence identity to an amino acid sequence encoded by a germline nucleotide human heavy chain J segment; and contains a V_{L} region that comprises a portion having at least 95%, 96%, 97%, 98%, 99%, or 100% sequence identity to an amino acid sequence encoded by a germline nucleotide human kappa or lambda chain V segment, and/or a portion having at least 95%, 96%, 97%, 98%, 99%, or 100% sequence identity to an amino acid sequence encoded by a germline nucleotide human kappa or lambda chain J segment. In some embodiments, the portion of the VH region corresponds to the CDR-H1, the CDR-H2 and/or the CDR-H3. In some embodiments, the portion of the VH region corresponds to the CDR-H1, the CDR-H2 and the CDR-H3. In some embodiments, the portion of the VL region corresponds to the CDR-L1, the CDR-L2 and/or the CDR-L3. In some embodiments, the portion of the VL region corresponds to the CDR-L1, the CDR-L2 and the CDR-L3.

Among the provided antibodies are monoclonal antibodies, including monoclonal antibody fragments.

In some embodiments, the anti-CD3 antibody, e.g., antigen-binding antibody fragment, contains a V_{H} region sequence that contains a CDR-H1, a CDR-H2 and a CDR-H3 as described and contains a V_{L} region sequence that contains a CDR-L1, a CDR-L2 and/or a CDR-L3 as described. Also among the provided antibodies and fragment thereof are those having sequences at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% identical to such a sequence, e.g., any of the CDR-H1, CDR-H2, CDR-H3, CDR-L1, CDR-L2, CDR-L3 sequences of the antibodies described herein. Also among the provided antibodies and fragment thereof are those having sequences at least at or about 85%, at or about 86%, at or about 87%, at or about 88%, at or about 89%, at or about 90%, at or about 91%, at or about 92%, at or about 93%, at or about 94%, at or about 95%, at or about 96%, at or about 97%, at or about 98%, or at or about 99% identical to such a sequence, e.g., any of the V_{H} and V_{L} sequences, respectively, of the antibodies described herein.

In some embodiments, provided are anti-CD3 antibodies, including antibody fragments, that have a heavy chain variable (V_{H}) region that comprises a heavy chain complementarity determining region 1 (CDR-H1) comprising the amino acid sequence set forth in SEQ ID NO: 198, a heavy chain complementarity determining region 2 (CDR-H2) comprising the amino acid sequence set forth in SEQ ID NO: 276, and a heavy chain complementarity determining region 2 (CDR-H3) comprising the amino acid sequence set forth in SEQ ID NO: 200; and a light chain variable (VL) region that comprises a light chain complementarity determining region 1 (CDR-L1) comprising the amino acid sequence set forth in SEQ ID NO: 24, a light chain complementarity determining region 2 (CDR-L2) comprising the amino acid sequence set forth in SEQ ID NO: 201, and a light chain complementarity determining region 3 (CDR-L3) comprising the amino acid sequence set forth within SEQ ID NO: 277.

In some embodiments, provided are anti-CD3 antibodies, including antibody fragments, that have a heavy chain variable (V_{H}) region that comprises a heavy chain complementarity determining region 1 (CDR-H1) comprising the amino acid sequence set forth in SEQ ID NO: 198, a heavy chain complementarity determining region 2 (CDR-H2) comprising the amino acid sequence set forth in SEQ ID NO: 199, and a heavy chain complementarity determining region 2 (CDR-H3) comprising the amino acid sequence set forth in SEQ ID NO: 200; and a light chain; and a light chain variable (VL) region that comprises a light chain complementarity determining region 1 (CDR-L1) comprising the amino acid sequence set forth in SEQ ID NO: 24, a light chain complementarity determining region 2 (CDR-L2) comprising the amino acid sequence set forth in SEQ ID NO: 201, and a light chain complementarity determining region 3 (CDR-L3) comprising the amino acid sequence set forth within SEQ ID NO: 106.

In some embodiments, among provided anti-CD3 antibodies, including antibody fragments, include a heavy chain variable (V_{H}) region that comprises a sequence of the consensus sequence QVQLVESGGGVVQPGX₁SLRLSCAASGFTFSX₂X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁L TX₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G or A or S; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 278); and a VL region sequence of the consensus sequence set forth in AIQLTQSPSSLSASVGDRVTITCRASQGISSALAWYQQKPGKAPKLLIYDASX₁LEX₂G VPSRFSGSGSGTDFTLTISSLQPEDFATYYCQQFX₃SYPRTFGX₄GTKVEIK, wherein X₁ is S or G, X₂ is N or S; X₃ is K or N; and X₄ is Q or C (SEQ ID NO: 197).

In some embodiments, among provided anti-CD3 antibodies, including antibody fragments, include a heavy chain variable (V_{H}) region that comprises a sequence of the consensus sequence QVQLVESGGGVVQPGX₁SLRLSCAASGFTFSX₂X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁L TX₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G or A; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 196) and a VL region comprising a sequence of the consensus sequence set forth in AIQLTQSPSSLSASVGDRVTITCRASQGISSALAWYQQKPGKAPKLLIYDASX₁LEX₂G VPSRFSGSGSGTDFTLTISSLQPEDFATYYCQQFX₃SYPRTFGX₄GTKVEIK, wherein X₁ is S or G, X₂ is N or S; X₃ is K or N; and X₄ is Q or C; (SEQ ID NO: 197).

In some embodiments, the CDR-H1 comprises the sequence set forth in any one of SEQ ID NOs: 19, 109, 202, or 203, the CDR-H2 comprises the sequence set forth in any one of SEQ ID NOs: 20, 107, 110, 112, or 204-208, and the CDR-H3 comprises the sequence set forth in any one of SEQ ID NOs: 21, 108, 111, 113, or 209-214; and the CDR-L1 comprises the sequence set forth in SEQ ID NOs: 24, the CDR-L2 comprises the sequence set forth in any one of SEQ ID NOs: 25, 114, 215, or 216, and the CDR-L3 comprises the sequence set forth in any one of SEQ ID NOs: 26 or 115.

In some embodiments, in the CD3 binding region, the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NO: 37. In some embodiments, in the CD3 binding region, the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NO: 39. In some embodiments, in the CD3 binding region, the VH region comprises the CDR-H1, the CDR-H2, and the CDR-H3 contained within SEQ ID NO: 40, and the VL region comprises the CDR-L1, the CDR-L2, and the CDR-L3 contained within SEQ ID NO: 39. In some embodiments, in the CD3 binding region, the VH region comprises the CDR-H1, the CDR-H2, and the CDR-H3 contained within SEQ ID NO: 42, and the VL region comprises the CDR-L1, the CDR-L2, and the CDR-L3 contained within SEQ ID NO: 43. In some embodiments, in the CD3 binding region, the VH region comprises the CDR-H1, the CDR-H2, and the CDR-H3 contained within SEQ ID NO: 22, and the VL region comprises the CDR-L1, the CDR-L2, and the CDR-L3 contained within SEQ ID NO: 27. In some embodiments, in the CD3 binding region, the VH region comprises the CDR-H1, the CDR-H2, and the CDR-H3 contained within SEQ ID NO: 68, and the VL region comprises the CDR-L1, the CDR-L2, and the CDR-L3 contained within SEQ ID NO: 39. In some embodiments, in the CD3 binding region, the VH region comprises the CDR-H1, the CDR-H2, and the CDR-H3 contained within SEQ ID NO: 70, and the VL region comprises the CDR-L1, the CDR-L2, and the CDR-L3 contained within SEQ ID NO: 43. In some embodiments, in the CD3 binding region, the VH region comprises the CDR-H1, the CDR-H2, and the CDR-H3 contained within SEQ ID NO: 72, and the VL region comprises the CDR-L1, the CDR-L2, and the CDR-L3 contained within SEQ ID NO: 73. In some embodiments, in the CD3 binding region, the VH region comprises the CDR-H1, the CDR-H2, and the CDR-H3 contained within SEQ ID NO: 74, and the VL region comprises the CDR-L1, the CDR-L2, and the CDR-L3 contained within SEQ ID NO: 39. In some embodiments, in the CD3 binding region, the VH region comprises the CDR-H1, the CDR-H2, and the CDR-H3 contained within SEQ ID NO: 78, and the VL region comprises the CDR-L1, the CDR-L2, and the CDR-L3 contained within SEQ ID NO: 39. In some embodiments, in the CD3 binding region, the VH region comprises the CDR-H1, the CDR-H2, and the CDR-H3 contained within SEQ ID NO: 80, and the VL region comprises the CDR-L1, the CDR-L2, and the CDR-L3 contained within SEQ ID NO: 39. In some embodiments, in the CD3 binding region, the VH region comprises the CDR-H1, the CDR-H2, and the CDR-H3 contained within SEQ ID NO: 82, and the VL region comprises the CDR-L1, the CDR-L2, and the CDR-L3 contained within SEQ ID NO: 39. In some embodiments, in the CD3 binding region, the VH region comprises the CDR-H1, the CDR-H2, and the CDR-H3 contained within SEQ ID NO: 84, and the VL region comprises the CDR-L1, the CDR-L2, and the CDR-L3 contained within SEQ ID NO: 39. In some embodiments, in the CD3 binding region, the VH region comprises the CDR-H1, the CDR-H2, and the CDR-H3 contained within SEQ ID NO: 86, and the VL region comprises the CDR-L1, the CDR-L2, and the CDR-L3 contained within SEQ ID NO: 39. In some embodiments, in the CD3 binding region, the VH region comprises the CDR-H1, the CDR-H2, and the CDR-H3 contained within SEQ ID NO: 159, and the VL region comprises the CDR-L1, the CDR-L2, and the CDR-L3 contained within SEQ ID NO: 43. In some embodiments, in the CD3 binding region, the VH region comprises the CDR-H1, the CDR-H2, and the CDR-H3 contained within SEQ ID NO: 195, and the VL region comprises the CDR-L1, the CDR-L2, and the CDR-L3 contained within SEQ ID NO: 39. In some embodiments, in the CD3 binding region, the VH region comprises the CDR-H1, the CDR-H2, and the CDR-H3 contained within SEQ ID NO: 41, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 64, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 65, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 66, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 67, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 69, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 71, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 75, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 76, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 77, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 79, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 81, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 83, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 85, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 87, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 90, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 91, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 92, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 93, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 94, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 95, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 96, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 97, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 98, and the VL region comprises the CDR-L1, the CDR-L2, and the CDR-L3 contained within SEQ ID NOs: 37, 39, or 43. In some embodiments, in the CD3 binding region, the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 39. In some embodiments, in the CD3 binding region, the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 99. In some embodiments, in the CD3 binding region, the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 43. In some embodiments, in the CD3 binding region, the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 100. In some embodiments, in the CD3 binding region, the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 101. In some embodiments, in the CD3 binding region, the VH region comprises the CDR-H1, the CDR-H2, and the CDR-H3 contained within SEQ ID NO: 42, and the VL region comprises the CDR-L1, the CDR-L2, and the CDR-L3 contained within SEQ ID NOs: 37. In some embodiments, in the CD3 binding region, the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NOs: 37. In some embodiments, in the CD3 binding region, the VH region comprises the CDR-H1, the CDR-H2, and the CDR-H3 contained within SEQ ID NO: 40, and the VL region comprises the CDR-L1, the CDR-L2, and the CDR-L3 contained within SEQ ID NOs: 37.

In some embodiments, in the CD3 binding domain, the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 109, 110, and 108, respectively, and the VL region comprises the CDR-L1, the CDR-L2, and the CDR-L3 comprising the sequence set forth in SEQ ID NOS: 24, 25, and 26, respectively. In some embodiments, in the CD3 binding domain the VH region comprises the CDR-H1, the CDR-H2, and the CDR-H3 comprising the sequence set forth in SEQ ID NOS: 19, 110, and 21, respectively, and the VL region comprises the CDR-L1, the CDR-L2, and the CDR-L3 comprising the sequence set forth in SEQ ID NOS: 24, 25, and 26, respectively. In some embodiments, in the CD3 binding domain the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 25, and 115, respectively. In some embodiments, in the CD3 binding domain the VH region comprises the CDR-H1, the CDR-H2, and the CDR-H3 comprising the sequence set forth in SEQ ID NOS: 19, 20, and 21, respectively, and the VL region comprises the CDR-L1, the CDR-L2, and the CDR-L3 comprising the sequence set forth in SEQ ID NOS: 24, 25, and 26, respectively. In some embodiments, in the CD3 binding domain the VH region comprises the CDR-H1, the CDR-H2, and the CDR-H3 comprising the sequence set forth in SEQ ID NOS: 19, 20, and 111, respectively, and the VL region comprises the CDR-L1, the CDR-L2, and the CDR-L3 comprising the sequence set forth in SEQ ID NOS: 24, 25, and 115, respectively. In some embodiments, in the CD3 binding domain the VH region comprises the CDR-H1, the CDR-H2, and the CDR-H3 comprising the sequence set forth in SEQ ID NOS: 19, 110, and 21, respectively, and the VL region comprises the CDR-L1, the CDR-L2, and the CDR-L3 comprising the sequence set forth in SEQ ID NOS: 24, 25, and 115, respectively. In some embodiments, in the CD3 binding domain the VH region comprises the CDR-H1, the CDR-H2, and the CDR-H3 comprising the sequence set forth in SEQ ID NOS: 19, 112, and 21, respectively, and the VL region comprises the CDR-L1, the CDR-L2, and the CDR-L3 comprising the sequence set forth in SEQ ID NOS: 24, 25, and 115, respectively. In some embodiments, in the CD3 binding domain the VH region comprises the CDR-H1, the CDR-H2, and the CDR-H3 comprising the sequence set forth in SEQ ID NOS: 19, 112, and 21, respectively, and the VL region comprises the CDR-L1, the CDR-L2, and the CDR-L3 comprising the sequence set forth in SEQ ID NOS: 24, 25, and 26, respectively. In some embodiments, in the CD3 binding domain the VH region comprises the CDR-H1, the CDR-H2, and the CDR-H3 comprising the sequence set forth in SEQ ID NOS: 19, 20, and 21, respectively, and the VL region comprises the CDR-L1, the CDR-L2, and the CDR-L3 comprising the sequence set forth in SEQ ID NOS: 24, 25, and 115, respectively. In some embodiments, in the CD3 binding domain the VH region comprises the CDR-H1, the CDR-H2, and the CDR-H3 comprising the sequence set forth in SEQ ID NOS: 19, 20, and 113, respectively, and the VL region comprises the CDR-L1, the CDR-L2, and the CDR-L3 comprising the sequence set forth in SEQ ID NOS: 24, 25, and 115, respectively. In some embodiments, in the CD3 binding domain the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 204, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 110, and 113, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 205, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 206, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 107, and 209, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 107, and 113, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 107, and 210, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 107, and 211, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 107, and 111, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 109, 107, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 202, 107, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 203, 107, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 107, and 212, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 107, and 213, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 107, and 214, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 107, and 21, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 207, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 208, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 110, and 210, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 202, 110, and 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 110, and 214, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 25, and 26, respectively. In some embodiments, in the CD3 binding domain the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 115, respectively. In some embodiments, in the CD3 binding domain the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 25, and 115, respectively. In some embodiments, in the CD3 binding domain the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 215, and 26, respectively. In some embodiments, in the CD3 binding domain the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 216, and 115, respectively. In some embodiments, in the CD3 binding domain the VH region comprises the CDR-H1, the CDR-H2, and the CDR-H3 comprising the sequence set forth in SEQ ID NOS: 19, 110, and 111, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 109, 110, 108, respectively, and the VL region comprises the CDR-L1, the CDR-L2, and the CDR-L3 comprising the sequence set forth in SEQ ID NOS: 24, 114, and 26, respectively. In some embodiments, in the CD3 binding domain the VH region comprises the CDR-H1, the CDR-H2, and the CDR-H3 comprising the sequence set forth in SEQ ID NOS: 19, 110, and 21, respectively, and the VL region comprises the CDR-L1, the CDR-L2, and the CDR-L3 comprising the sequence set forth in SEQ ID NOS: 24, 114, and 26, respectively.

In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39 the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 22, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 68, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 70, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 72, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 73. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 74, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 78, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 80, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 82, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 84, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 86, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 159, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 195, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 64, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 65, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 66, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 67, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 69, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 71, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 75, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 76, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 77, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 79, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 81, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 83, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 85, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 87, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 90, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 91, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 92, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 93, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 94, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 95, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 96, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 97, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 98, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 99. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 100. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 101. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37. In some embodiments, in the CD3 binding domain, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37.

In some embodiments, in the CD3 binding domain, the VH region and the VL region comprise the sequences set forth in SEQ ID NO: 36 and 37, respectively. In some embodiments, in the CD3 binding domain, the VH region and the VL region comprise the sequences set forth in SEQ ID NO: 38 and 39, respectively. In some embodiments, in the CD3 binding domain, the VH region and the VL region comprise the sequences set forth in SEQ ID NO: 40 and 39, respectively. In some embodiments, in the CD3 binding domain, the VH region and the VL region comprise the sequences set forth in SEQ ID NO: 42 and 43, respectively. In some embodiments, in the CD3 binding domain, the VH region and the VL region comprise the sequences set forth in SEQ ID NO: 22 and 27, respectively. In some embodiments, in the CD3 binding domain, the VH region and the VL region comprise the sequences set forth in SEQ ID NO: 68 and 39, respectively. In some embodiments, in the CD3 binding domain, the VH region and the VL region comprise the sequences set forth in SEQ ID NO: 70 and 43, respectively. In some embodiments, in the CD3 binding domain, the VH region and the VL region comprise the sequences set forth in SEQ ID NO: 72 and 73, respectively. In some embodiments, in the CD3 binding domain, the VH region and the VL region comprise the sequences set forth in SEQ ID NO: 74 and 39, respectively. In some embodiments, in the CD3 binding domain, the VH region and the VL region comprise the sequences set forth in SEQ ID NO: 78 and 39, respectively. In some embodiments, in the CD3 binding domain, the VH region and the VL region comprise the sequences set forth in SEQ ID NO: 80 and 39, respectively. In some embodiments, in the CD3 binding domain, the VH region and the VL region comprise the sequences set forth in SEQ ID NO: 82 and 39, respectively. In some embodiments, in the CD3 binding domain, the VH region and the VL region comprise the sequences set forth in SEQ ID NO: 84 and 39, respectively. In some embodiments, in the CD3 binding domain, the VH region and the VL region comprise the sequences set forth in SEQ ID NO: 86 and 39, respectively. In some embodiments, in the CD3 binding domain, the VH region and the VL region comprise the sequences set forth in SEQ ID NO: 159 and 43, respectively. In some embodiments, in the CD3 binding domain, the VH region and the VL region comprise the sequences set forth in SEQ ID NO: 195 and 39, respectively. In some embodiments, in the CD3 binding domain, the VH region and the VL region comprise the sequences set forth in SEQ ID NO: 41 and 37, respectively. In some embodiments, in the CD3 binding domain, the VH region and the VL region comprise the sequences set forth in SEQ ID NO: 64 and 37, respectively. In some embodiments, in the CD3 binding domain, the VH region and the VL region comprise the sequences set forth in SEQ ID NO: 65 and 37, respectively. In some embodiments, in the CD3 binding domain, the VH region and the VL region comprise the sequences set forth in SEQ ID NO: 66 and 37, respectively. In some embodiments, in the CD3 binding domain, the VH region and the VL region comprise the sequences set forth in SEQ ID NO: 67 and 37, respectively. In some embodiments, in the CD3 binding domain, the VH region and the VL region comprise the sequences set forth in SEQ ID NO: 69 and 37, respectively. In some embodiments, in the CD3 binding domain, the VH region and the VL region comprise the sequences set forth in SEQ ID NO: 71 and 37, respectively. In some embodiments, in the CD3 binding domain, the VH region and the VL region comprise the sequences set forth in SEQ ID NO: 75 and 37, respectively. In some embodiments, in the CD3 binding domain, the VH region and the VL region comprise the sequences set forth in SEQ ID NO: 76 and 37, respectively. In some embodiments, in the CD3 binding domain, the VH region and the VL region comprise the sequences set forth in SEQ ID NO: 77 and 37, respectively. In some embodiments, in the CD3 binding domain, the VH region and the VL region comprise the sequences set forth in SEQ ID NO: 79 and 37, respectively. In some embodiments, in the CD3 binding domain, the VH region and the VL region comprise the sequences set forth in SEQ ID NO: 81 and 37, respectively. In some embodiments, in the CD3 binding domain, the VH region and the VL region comprise the sequences set forth in SEQ ID NO: 83, and 37, respectively. In some embodiments, in the CD3 binding domain, the VH region and the VL region comprise the sequences set forth in SEQ ID NO: 85 and 37, respectively. In some embodiments, in the CD3 binding domain, the VH region and the VL region comprise the sequences set forth in SEQ ID NO: 87 and 37, respectively. In some embodiments, in the CD3 binding domain, the VH region and the VL region comprise the sequences set forth in SEQ ID NO: 90 and 37, respectively. In some embodiments, in the CD3 binding domain, the VH region and the VL region comprise the sequences set forth in SEQ ID NO: 91 and 37, respectively. In some embodiments, in the CD3 binding domain, the VH region and the VL region comprise the sequences set forth in SEQ ID NO: 92 and 37, respectively. In some embodiments, in the CD3 binding domain, the VH region and the VL region comprise the sequences set forth in SEQ ID NO: 93 and 37, respectively. In some embodiments, in the CD3 binding domain, the VH region and the VL region comprise the sequences set forth in SEQ ID NO: 94 and 37, respectively. In some embodiments, in the CD3 binding domain, the VH region and the VL region comprise the sequences set forth in SEQ ID NO: 95 and 37, respectively. In some embodiments, in the CD3 binding domain, the VH region and the VL region comprise the sequences set forth in SEQ ID NO: 96 and 37, respectively. In some embodiments, in the CD3 binding domain, the VH region and the VL region comprise the sequences set forth in SEQ ID NO: 97 and 37, respectively. In some embodiments, in the CD3 binding domain, the VH region and the VL region comprise the sequences set forth in SEQ ID NO: 98 and 37, respectively. In some embodiments, in the CD3 binding domain, the VH region and the VL region comprise the sequences set forth in SEQ ID NO: 36 and 39, respectively. In some embodiments, in the CD3 binding domain, the VH region and the VL region comprise the sequences set forth in SEQ ID NO: 36 and 99, respectively. In some embodiments, in the CD3 binding domain, the VH region and the VL region comprise the sequences set forth in SEQ ID NO: 36 and 43, respectively. In some embodiments, in the CD3 binding domain, the VH region and the VL region comprise the sequences set forth in SEQ ID NO: 36 and 100, respectively. In some embodiments, in the CD3 binding domain, the VH region and the VL region comprise the sequences set forth in SEQ ID NO: 36 and 101, respectively. In some embodiments, in the CD3 binding domain, the VH region and the VL region comprise the sequences set forth in SEQ ID NO: 42 and 37, respectively. In some embodiments, in the CD3 binding domain, the VH region and the VL region comprise the sequences set forth in SEQ ID NO: 38 and 37, respectively. In some embodiments, in the CD3 binding domain, the VH region and the VL region comprise the sequences set forth in SEQ ID NO: 40 and 37, respectively.

The disclosed binding molecules include anti-CD3 antibodies, including antigen binding fragments, comprising specified paratope resides that interact with the human CD3 epsilon. For example, in certain embodiments, the antigen binding domain that specifically binds human CD3 epsilon comprises a heavy chain variable region having at least one amino acid, in the specified position selected from the group consisting of G33, H35, I51, W52, Y53, S99, Y101, 1103, R106, Q109, and Y110 of SEQ ID NO:36 and a light chain variable region having at least one amino acid in the specified position selected from the group consisting of S30, F91, K92, Y94, and R96 of SEQ ID NO:37.

In another embodiment, the antigen binding domain that specifically binds human CD3 epsilon comprises a heavy chain variable region having amino acids in the specified positions of G33, H35, 151, W52, Y53, S99, Y101, 1103, R106, Q109, and Y110 of SEQ ID NO:36 and a light chain variable region having amino acids in the specified positions of S30, F91, K92, Y94, and R96 of SEQ ID NO:37.

In some embodiments, provided are anti-CD3 antibodies, including antibody fragments, that have a heavy chain variable (VH) region that comprises a CDR-H1 set forth in SEQ ID NO: 102, a CDR-H2 set forth in SEQ ID NO: 103, and a CDR-H3 set forth in SEQ ID NO: 104; and a VL region that comprises a CDR-L1 set forth in SEQ ID NO: 24, a CDR-L2 set forth in SEQ ID NO: 105, and a CDR-L3 set forth within SEQ ID NO: 106.

In some embodiments, among provided anti-CD3 antibodies, including antibody fragments, include a V_{H} region that comprises a sequence of the consensus sequence QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTRYY X₈X₉GX₁₀X₁₁VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is Q or S; X₉ is Y or H; X₁₀ is I or L; X₁₁ is N or D; (SEQ ID NO: 192).

In some embodiments, among provided anti-CD3 antibodies, including antibody fragments, include a V_{L} region that comprises a sequence of the consensus sequence AIQLTQSPSSLSASVGDRVTITCRASQGISSALAWYQQKPGKAPKLLIYDASSLESGVP SRFSGSGSGTDFTLTISSLQPEDFATYYCQQFX₁SYPRTFGX₂GTKVEIK, wherein X₁=K or N
X₂=Q or C; (SEQ ID NO: 193).

In some embodiments, among provided anti-CD3 antibodies, including antibody fragments, include a V_{H} region that comprises a sequence of the consensus sequence QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTX₈X₉YX₁₀X₁₁GX₁₂X₁₃VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is R or G; X₉ is Y or H; X₁₀ is Q or S; X₁₁ is Y or H; X₁₂ is I or L; and X₁₃ is N or D; (SEQ ID NO: 116).

In some embodiments, among provided anti-CD3 antibodies, including antibody fragments, include a V_{L} region that comprises a sequence of the consensus sequence AIQLTQSPSSLSASVGDRVTITCRASQGISSALAWYQQKPGKAPKLLIYDASSLEX₁GV PSRFSGSGSGTDFTLTISSLQPEDFATYYCQQFX₂SYPRTFGX₃GTKVEIK, wherein X₁ is N or S; X₂ is K or N; X₃ is Q or C; (SEQ ID NO: 117).

In some embodiments, provided anti-CD3 antibodies, including antibody fragments, comprises a V_{H} region comprising a CDR-H1, a CDR-H2 and a CDR-H3 present in (or contained in) a V_{H} sequence set forth in any one of SEQ ID NOS: 36, 38, 40, 42, 22, 68, 70, 72, 74, 78, 80, 82, 84, 86, 159, or 195, and a VL region comprising CDR-L1, a CDR-L2, and a CDR-L3 present in (or contained in) a V_{L} sequence set forth in any one of SEQ ID NO: 27, 37, 39, 43, or 73.

In some embodiments, the V_{H} region of an antibody or antigen-binding fragment thereof comprises a CDR-H1, a CDR-H2 and a CDR-H3 according to Kabat numbering. In some embodiments, the V_{L} region of an antibody or antigen-binding fragment thereof comprises a CDR-L1, a CDR-L2 and a CDR-L3 according to Kabat numbering.

In some embodiments, the CDR-H1 comprises the amino acid sequence set forth in set forth in any one of SEQ ID NOs: 19 or 109, the CDR-H2 comprises the amino acid sequence set forth in any one of SEQ ID NOs: 20, 107, 110, or 112, and the CDR-H3 comprises the amino acid sequence set forth in any one of SEQ ID NOs: 21, 108, 111, or 113. In some embodiments, the CDR-L1 comprises the amino acid sequence set forth in SEQ ID NOs: 24, the CDR-L2 comprises the amino acid sequence set forth in any one of SEQ ID NOs: 25 or 114, and the CDR-L3 comprises the amino acid sequence set forth in any one of SEQ ID NOs: 26 or 115.

In some embodiments, the V_{H} region comprises the CDR-H1, the CDR-H2, and the CDR-H3 contained within SEQ ID NO: 36, and the V_{L} region comprises the CDR-L1, the CDR-L2, and the CDR-L3 contained within SEQ ID NO: 37. In some embodiments, the V_{H} region comprises the CDR-H1, the CDR-H2, and the CDR-H3 comprising the sequence set forth in SEQ ID NOS: 19, 107, and 108, respectively, and the V_{L} region comprises the CDR-L1, the CDR-L2, and the CDR-L3 comprising the sequence set forth in SEQ ID NOS: 24, 114, and 26, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37. In some embodiments, the V_{H}region and the V_{L} region comprise the sequences set forth in SEQ ID NO: 36 and 37, respectively. In some embodiments, the CD3 binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 250. In some embodiments, the binding domain comprises an the amino acid sequence of SEQ ID NO: 250.

In some embodiments, among provided binding molecules provided herein are binding molecules in which an antibody, including antigen binding fragment, disclosed herein may comprise one or more amino acid substitutions, insertions and/or deletions in the framework and/or CDR regions of the heavy and light chain variable domains as compared to a reference human antibody or compared to the corresponding germline sequences from which the antibodies were derived. Such mutations can be readily ascertained by comparing the amino acid sequences disclosed herein to the reference sequence or to germline sequences available from, for example, public antibody sequence databases.

In some embodiments, the binding molecules provided herein include antibodies, and antigen-binding fragments thereof, which are derived from any of the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). A person of ordinary skill in the art, starting with the heavy and light chain variable region sequences disclosed herein, can easily produce numerous antibodies and antigen-binding fragments which comprise one or more individual germline mutations or combinations thereof. In certain embodiments, all of the framework and/or CDR residues within the VH and/or VL domains are mutated back to the residues found in the original germline sequence from which the antibody was derived. In other embodiments, only certain residues are mutated back to the original germline sequence, e.g., only the mutated residues found within the first 8 amino acids of FR1 or within the last 8amino acids of FR4, or only the mutated residues found within CDR1, CDR2 or CDR3. In other embodiments, one or more of the framework and/or CDR residue(s) are mutated to the corresponding residue(s) of a different germline sequence (i.e., a germline sequence that is different from the germline sequence from which the antibody was originally derived). Furthermore, the antibodies of the present invention may contain any combination of two or more germline mutations within the framework and/or CDR regions, e.g., wherein certain individual residues are mutated to the corresponding residue of a particular germline sequence while certain other residues that differ from the original germline sequence are maintained or are mutated to the corresponding residue of a different germline sequence. Once obtained, antibodies and antigen-binding fragments that contain one or more germline mutations can be easily tested for one or more desired property such as, improved binding specificity, increased binding affinity, improved or enhanced antagonistic or agonistic biological properties (as the case may be), reduced immunogenicity, etc. Antibodies and antigen-binding fragments obtained in this general manner are encompassed within the present invention.

In some embodiments, the binding molecules provided herein include antibodies, and antigen-binding fragments thereof, comprising variants in which the framework and/or CDR regions are mutated with one or more conservative substitutions. For example, the present invention includes antibodies and antibody fragments having framework and/or CDR regions amino acid sequences with, e.g., 10 or fewer, 8 or fewer, 6 or fewer, 4 or fewer, etc. conservative amino acid substitutions relative to any of the framework and/or CDR regions amino acid sequences set forth in the tables herein.

In some embodiments, the anti-CD3 antibody can be chosen as having a particular or desired binding affinity. In some embodiments, the anti-CD3 antibody has a CD3 dissociation constant of <1 μM, <100 nM, <10 nM, <1 nM, <0.1 nM, <0.01 nM, or <0.001 nM (e.g., 10⁻⁸M or less, e.g., from 10⁻⁸M to 10⁻¹³M, e.g., from 10⁻⁹M to 10⁻¹³ M). In some embodiments, the provided binding molecules, including bispecific antibodies, incorporate anti-CD3 antibodies that exhibit a medium binding affinity for binding to CD3. In some embodiments, an anti-CD3 antibody exhibits a binding affinity (Kd) that is greater than 5 nM. In some embodiments, an anti-CD3 antibody incorporated in bispecific antibodies herein exhibits a binding affinity (Kd) that is greater than 10 nM. In some embodiments, the Kd of an anti-CD3 antibody is at or about 5 nM, 7.5 nM, 10 nM, 15 nM, 20 nM, 25 nM, 30 nM, 40 nM, 50 nM, 60 nM, 70 nM, 80 nM, 90 nM, 100 nM, or any value between any of the foregoing. In some embodiments, the Kd of an anti-CD3 antibody is between 10 nM and 70 nM.

In a particular embodiments, among provided antibodies are antibodies, including antigen binding fragments, that comprise one or more amino acid substitution in the framework and/or CDR region relative to the antibody designated CD3-1, for example, an anti-CD3 antibody comprising in which the V_{H}region and the V_{L} region comprise the sequences set forth in SEQ ID NO: 36 and 37, respectively.

In some embodiments, the VH region comprises one or more amino acid residues selected from the group consisting of: an asparagine (N) at position 31, an alanine (A) at position 55, a tyrosine (Y) at position 80, a glycine (G) at position 106, a histidine (H) at position 107, a serine (S) at position 109, a histidine (H) at position 110, a leucine (L) at position 112, and an aspartic acid (D) at position 113 or any combination thereof, wherein the numbering is relative to SEQ ID NO: 36. In some embodiments, the VL region comprises one or more amino acid residues selected from the group consisting of: a serine (S) at position 56 an asparagine (N) at position 92, and a cysteine (C) at position 100, an asparagine (N) at position 108, or any combination thereof, wherein the numbering is relative to SEQ ID NO: 37. In some embodiments, the V_{H} region comprises one or more amino acid residues selected from the group consisting of: an asparagine (N) at position 31, an alanine (A) at position 55, a tyrosine (Y) at position 80, a glycine (G) at position 106, a histidine (H) at position 107, a serine (S) at position 109, a histidine (H) at position 110, a leucine (L) at position 112, and an aspartic acid (D) at position 113 or any combination thereof, wherein the numbering is relative to SEQ ID NO: 36, and the VL region comprises one or more amino acid residues selected from the group consisting of: a serine (S) at position 56, an asparagine (N) at position 92, and a cysteine (C) at position 100, an asparagine (N) at position 108, or any combination thereof, wherein the numbering is relative to SEQ ID NO: 37. In some of any such embodiments, the Kd of an anti-CD3 antibody is between 10 nM and 70 nM, such as 10 nM, 15 nM, 20 nM, 25 nM, 30 nM, 40 nM, 50 nM, 60 nM, or 70 nM, or any value between any of the foregoing.

In some embodiments, the V_{H} region comprises amino acid residues alanine (A) at position 55 and tyrosine (Y) at position 80, wherein the numbering is relative to SEQ ID NO: 24. In some embodiments, the V_{H} region comprises amino acid residues asparagine (N) at position 31, alanine (A) at position 55, and tyrosine (Y) at position 80, wherein the numbering is relative to SEQ ID NO: 36. In some embodiments, the V_{H} region comprises amino acid residues alanine (A) at position 55, tyrosine (Y) at position 80, leucine (L) at position 112, and aspartic acid (D) at position 113, wherein the numbering is relative to SEQ ID NO: 36. In some embodiments, the V_{H} region comprises amino acid residues alanine (A) at position 55, tyrosine (Y) at position 80, serine (S) at position 109, and histidine (H) at position 110, wherein the numbering is relative to SEQ ID NO: 36. In some embodiments, the VL region comprises serine (S) at position 56, wherein the numbering is relative to SEQ ID NO:37. In some embodiments, the VL region comprises serine (S) at position 56 and asparagine (N) at position 92, wherein the numbering is relative to SEQ ID NO: 37. In some embodiments, the V_{H} region comprises amino acid residues asparagine (N) at position 31, alanine (A) at position 55, and tyrosine (Y) at position 80, wherein the numbering is relative to SEQ ID NO: 36, and the VL region comprises serine (S) at position 56, wherein the numbering is relative to SEQ ID NO:37. In some embodiments, the V_{H} region comprises amino acid residues alanine (A) at position 55, tyrosine (Y) at position 80, leucine (L) at position 112, and aspartic acid (D) at position 113, wherein the numbering is relative to SEQ ID NO: 36, and the VL region comprises serine (S) at position 56, wherein the numbering is relative to SEQ ID NO:37. In some embodiments, the VH region comprises amino acid residues alanine (A) at position 55, tyrosine (Y) at position 80, serine (S) at position 109, and histidine (H) at position 110, wherein the numbering is relative to SEQ ID NO: 36, and the VL region comprises serine (S) at position 56 and asparagine (N) at position 92, wherein the numbering is relative to SEQ ID NO: 37. In some of any such embodiments, the Kd of an anti-CD3 antibody is between 10 nM and 70 nM, such as 10 nM, 15 nM, 20 nM, 25 nM, 30 nM, 40 nM, 50 nM, 60 nM, or 70 nM, or any value between any of the foregoing. In some of any such embodiments, the anti-CD3 antibody has a Kd of 5 to 75 nM to CD3. In some of any such embodiments, the anti-CD3 antibody has a Kd of 10 to 70 nM to CD3.

In some embodiments, the V_{H} region comprises the CDR-H1, the CDR-H2, and the CDR-H3 contained within SEQ ID NO: 38, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOS: 109, 110 and 108, respectively, and the V_{L} region comprises the CDR-L1, the CDR-L2, and the CDR-L3 comprising the sequence set forth in SEQ ID NOS: 24, 25 and 26, respectively. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the V_{H} region comprises amino acid residues asparagine (N) at position 31, alanine (A) at position 55, and tyrosine (Y) at position 80, wherein the numbering is relative to SEQ ID NO: 36, and the VL region comprises serine (S) at position 56, wherein the numbering is relative to SEQ ID NO:37. In some embodiments, the V_{H}region and the V_{L} region comprise the sequences set forth in SEQ ID NO: 38 and 39, respectively.

In some embodiments, the V_{H} region comprises the CDR-H1, the CDR-H2, and the CDR-H3 contained within SEQ ID NO: 40, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOS: 19, 110 and 21, respectively, and the V_{L} region comprises the CDR-L1, the CDR-L2, and the CDR-L3 comprising the sequence set forth in SEQ ID NOS: 24, 25 and 26, respectively. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the V_{H} region comprises amino acid residues alanine (A) at position 55, tyrosine (Y) at position 80, leucine (L) at position 112, and aspartic acid (D) at position 113, wherein the numbering is relative to SEQ ID NO: 36, and the VL region comprises serine (S) at position 56, wherein the numbering is relative to SEQ ID NO:37. In some embodiments, the V_{H} region and the V_{L} region comprise the sequences set forth in SEQ ID NO: 40 and 39, respectively. In some embodiments, the binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 251. In some embodiments, the binding domain comprises an the amino acid sequence of SEQ ID NO: 251.

In some embodiments, the V_{H} region comprises the CDR-H1, the CDR-H2, and the CDR-H3 contained within SEQ ID NO: 42, and the V_{L} region comprises the CDR-L1, the CDR-L2, and the CDR-L3 contained within SEQ ID NO: 43. In some embodiments, the V_{H} region comprises the CDR-H1, the CDR-H2, and the CDR-H3 comprising the sequence set forth in SEQ ID NOS: 19, 110, 108, respectively, and the V_{L} region comprises the CDR-L1, the CDR-L2, and the CDR-L3 comprising the sequence set forth in SEQ ID NOS: 24, 25, and 115, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43. In some embodiments, the VH region comprises amino acid residues alanine (A) at position 55, tyrosine (Y) at position 80, serine (S) at position 109, and histidine (H) at position 110, wherein the numbering is relative to SEQ ID NO: 36, and the VL region comprises serine (S) at position 56 and asparagine (N) at position 92, wherein the numbering is relative to SEQ ID NO: 37. In some embodiments, the V_{H}region and the V_{L} region comprise the sequences set forth in SEQ ID NO: 42 and 43, respectively.

In some embodiments, the provided anti-CD3 antibodies or antigen-binding fragments thereof low polyreactivity. As used herein, an antibody or antigen-binding fragment thereof that exhibits “low polyreactivity” exhibits low non-specific binding to extracellular matrix proteins. Thus, in some embodiments, the provided anti-CD3 antibodies and antigen-binding fragments thereof exhibit low polyreactivity to extracellular matrix (ECM) proteins. This is advantageous because low polyreactivity with ECM proteins can reduce off-target effects caused by the antibody binding to ECM proteins, and can reduce or avoid the negative effects that polyreactivity can have on pharmacokinetics and bioavailability of the antibody.

Accordingly, in some embodiments, the anti-CD3 antibody or antigen-binding fragment thereof exhibits low non-specific binding to extracellular matrix proteins, such as by having an extracellular matrix (ECM) score of less than 14, less than 13, less than 12, less than 11, or less than 10. Low polyreactivity is advantageous because it can, e.g., reduce off-target effects caused by the antibody binding to extracellular matrix proteins, for instance; and it can also reduce or avoid the negative effects that polyreactivity can have on pharmacokinetics and bioavailability of the therapeutic antibody. Polyreactivity can be assessed by, for instance, reference to an ECM score that is calculated based on an ECM ELISA assay, where the ECM score is calculated by dividing the absorbance value of an ECM-coated sample well containing the antibody or antigen-binding fragment thereof by the absorbance of an ECM-coated sample well that does not contain the antibody or antigen-binding fragment thereof. In some embodiments, an ECM score of 12 at a concentration of 1 μM antibody or antigen-binding fragment thereof is used to determine whether an antibody or antigen-binding fragment thereof has a high or low ECM score, with a high ECM score being at or higher than 12, and a low ECM score being less than 12. In some embodiments, the anti-CD3 antibody or antigen-binding fragment thereof has an ECM score of less than 14, less than 13, less than 12, less than 11, or less than 10. In some embodiments, the anti-CD3 antibody or antigen-binding fragment thereof has an ECM score of less than 12.

In some embodiments, the VH region comprises one or more amino acid residues selected from glycine (G) at position 16, a glutamic acid (E) at position 16, a threonine (T) at position 58, an aspartic acid (D) at position 58, a glutamic acid (E) at position 76, a serine (S) at position 76, wherein the numbering is relative to SEQ ID NO: 36. In some embodiments, the V_{H} region comprises glycine (G) at position 16, wherein the numbering is relative to SEQ ID NO: 36. In some embodiments, the VH region comprises a threonine (T) at position 58, wherein the numbering is relative to SEQ ID NO: 36. In some embodiments, the V_{H} region comprises glycine (G) at position 16 and a threonine (T) at position 58, wherein the numbering is relative to SEQ ID NO: 36. In some of any such embodiments, the anti-CD3 antibody or antigen-binding fragment thereof has a low polyreactivity with an ECM score of less than 14, less than 13, less than 12, less than 11, or less than 10. In particular embodiments, the ECM score is less than 12.

In some embodiments, any of the provided antibodies or antigen-binding fragments are single chain variable fragments (scFv). In some embodiments, the scFv is a disulfide-stablized scFv (dsFv) wherein the VH includes a first engineered cysteine residue and the VL includes a second engineered cysteine residue and wherein the first engineered cysteine residue and the second engineered cysteine residue form a disulfide bond. In some embodiments, the first engineered cysteine residue is at position 44 (e.g., G44C amino acid substitution) in the VH, wherein the numbering is relative to SEQ ID NO: 36. In some embodiments, the second engineered cysteine residue is at position 100 (e.g., Q100C amino acid substitution) in the VL, wherein the numbering is relative to SEQ ID NO: 37. In some embodiments, the first engineered cysteine residue is at position 44 (e.g, G44C amino acid substitution) in the VH (wherein the numbering is relative to SEQ ID NO: 36) and the second engineered cysteine residue is at position 100 (e.g., Q100C amino acid substitution) in the VL (wherein the numbering is relative to SEQ ID NO: 37).

In some embodiments, the V_{H} region comprises the CDR-H1, the CDR-H2, and the CDR-H3 contained within SEQ ID NO: 22, and the V_{L} region comprises the CDR-L1, the CDR-L2, and the CDR-L3 contained within SEQ ID NO: 27. In some embodiments, the V_{H} region comprises the CDR-H1, the CDR-H2, and the CDR-H3 comprising the sequence set forth in SEQ ID NOS: 19, 20, and 21, respectively, and the V_{L} region comprises the CDR-L1, the CDR-L2, and the CDR-L3 comprising the sequence set forth in SEQ ID NOS: 24, 25, and 26, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 22, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27. In some embodiments, the VH comprises glycine (G) at position 16, cysteine (C) at position 44, a threonine (T) at position 58, alanine (A) at position 55, tyrosine (Y) at position 80, leucine (L) at position 112, and aspartic acid (D) at position 113 wherein the numbering is relative to SEQ ID NO: 36, and the VL region comprises serine (S) at position 56 and cysteine (C) at position 100, wherein the numbering is relative to SEQ ID NO: 37. In some embodiments, the V_{H} region and the V_{L} region comprise the sequences set forth in SEQ ID NO: 22 and 27, respectively. In some embodiments, the CD3 binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 246. In some embodiments, the CD3 binding domain comprises an amino acid sequence of SEQ ID NO: 246.

In some embodiments, the V_{H} region comprises the CDR-H1, the CDR-H2, and the CDR-H3 contained within SEQ ID NO: 68, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOS: 19, 20, and 21, respectively, and the V_{L} region comprises the CDR-L1, the CDR-L2, and the CDR-L3 comprising the sequence set forth in SEQ ID NOS: 24, 25, and 26, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 68, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the VH comprises amino acid residues glycine (G) at position 16, alanine (A) at position 55, threonine (T) at position 58, tyrosine (Y) at position 80, leucine (L) at position 112, and aspartic acid (D) at position 113, wherein the numbering is relative to SEQ ID NO: 36, and the VL region comprises serine (S) at position 56, wherein the numbering is relative to SEQ ID NO: 37. In some embodiments, the V_{H} region and the V_{L} region comprise the sequences set forth in SEQ ID NO: 68 and 39, respectively.

In some embodiments, the V_{H} region comprises the CDR-H1, the CDR-H2, and the CDR-H3 contained within SEQ ID NO: 70, and the V_{L} region comprises the CDR-L1, the CDR-L2, and the CDR-L3 contained within SEQ ID NO: 43. In some embodiments, the V_{H} region comprises the CDR-H1, the CDR-H2, and the CDR-H3 comprising the sequence set forth in SEQ ID NOS: 19, 20 and 111, respectively, and the V_{L} region comprises the CDR-L1, the CDR-L2, and the CDR-L3 comprising the sequence set forth in SEQ ID NOS: 24, 25 and 115, respectively. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 70, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43. In some embodiments, the VH comprises amino acid residues glycine (G) at position 16, alanine (A) at position 55, threonine (T) at position 58, tyrosine (Y) at position 80, serine (S) at position 109, and histidine (H) at position 110, wherein the numbering is relative to SEQ ID NO: 36, and the VL region comprises serine (S) at position 56 and asparagine (N) at position 92, wherein the numbering is relative to SEQ ID NO: 37. In some embodiments, the V_{H} region and the V_{L} region comprise the sequences set forth in SEQ ID NO: 70 and 43, respectively.

In some embodiments, the V_{H} region comprises the CDR-H1, the CDR-H2, and the CDR-H3 contained within SEQ ID NO: 72, and the V_{L} region comprises the CDR-L1, the CDR-L2, and the CDR-L3 contained within SEQ ID NO: 73. In some embodiments, the V_{H} region comprises the CDR-H1, the CDR-H2, and the CDR-H3 comprising the sequence set forth in SEQ ID NOS: 19, 20 and 111, respectively, and the V_{L} region comprises the CDR-L1, the CDR-L2, and the CDR-L3 comprising the sequence set forth in SEQ ID NOS: 24, 25 and 115, respectively. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 72, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 73. In some embodiments, the VH comprises amino acid residues glycine (G) at position 16, cysteine (C) at position 44, alanine (A) at position 55, threonine (T) at position 58, tyrosine (Y) at position 80, serine (S) at position 109, and histidine (H) at position 110, wherein the numbering is relative to SEQ ID NO: 36, and the VL region comprises serine (S) at position 56, asparagine (N) at position 92 and cysteine (C) at position 100, wherein the numbering is relative to SEQ ID NO: 37. In some embodiments, the V_{H} region and the V_{L} region comprise the sequences set forth in SEQ ID NO: 72 and 73, respectively. In some embodiments, the CD3 binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 247. In some embodiments, the CD3 binding domain comprises the amino acid sequence of SEQ ID NO: 247.

In some embodiments, the V_{H} region comprises the CDR-H1, the CDR-H2, and the CDR-H3 contained within SEQ ID NO: 74, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOS: 19, 110 and 21, respectively, and the V_{L} region comprises the CDR-L1, the CDR-L2, and the CDR-L3 comprising the sequence set forth in SEQ ID NOS: 24, 25 and 26, respectively. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 74, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the VH comprises amino acid residues glycine (G) at position 16, alanine (A) at position 55, tyrosine (Y) at position 80, leucine (L) at position 112, and aspartic acid (D) at position 113, wherein the numbering is relative to SEQ ID NO: 36, and the VL region comprises serine (S) at position 56, wherein the numbering is relative to SEQ ID NO: 37. In some embodiments, the V_{H} region and the V_{L} region comprise the sequences set forth in SEQ ID NO: 74 and 39, respectively.

In some embodiments, the V_{H} region comprises the CDR-H1, the CDR-H2, and the CDR-H3 contained within SEQ ID NO: 195, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOS: 19, 110 and 21, respectively, and the V_{L} region comprises the CDR-L1, the CDR-L2, and the CDR-L3 comprising the sequence set forth in SEQ ID NOS: 24, 25 and 26, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 195, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the VH comprises amino acid residues a glutamic acid (E) at position 16, alanine (A) at position 55, tyrosine (Y) at position 80, leucine (L) at position 112, and aspartic acid (D) at position 113, wherein the numbering is relative to SEQ ID NO: 36, and the VL region comprises serine (S) at position 56, wherein the numbering is relative to SEQ ID NO: 37. In some embodiments, the V_{H}region and the V_{L} region comprise the sequences set forth in SEQ ID NO: 195 and 39, respectively.

In some embodiments, the V_{H} region comprises the CDR-H1, the CDR-H2, and the CDR-H3 contained within SEQ ID NO: 78, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOS: 19, 112 and 21, respectively, and the V_{L} region comprises the CDR-L1, the CDR-L2, and the CDR-L3 comprising the sequence set forth in SEQ ID NOS: 24, 25 and 26, respectively. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 78, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the VH comprises amino acid residues a glutamic acid (E) at position 16, alanine (A) at position 55, aspartic acid (D) at position 58, tyrosine (Y) at position 80, leucine (L) at position 112, and aspartic acid (D) at position 113, wherein the numbering is relative to SEQ ID NO: 36, and the VL region comprises serine (S) at position 56, wherein the numbering is relative to SEQ ID NO: 37. In some embodiments, the V_{H}region and the V_{L} region comprise the sequences set forth in SEQ ID NO: 78 and 39, respectively.

In some embodiments, the V_{H} region comprises the CDR-H1, the CDR-H2, and the CDR-H3 contained within SEQ ID NO: 80, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOS: 19, 20 and 21, respectively, and the V_{L} region comprises the CDR-L1, the CDR-L2, and the CDR-L3 comprising the sequence set forth in SEQ ID NOS: 24, 25 and 26, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 80, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the VH comprises amino acid residues a glycine (G) at position 16, alanine (A) at position 55, threonine (T) at position 58, serine (S) at position 76, tyrosine (Y) at position 80, leucine (L) at position 112, and aspartic acid (D) at position 113, wherein the numbering is relative to SEQ ID NO: 36, and the VL region comprises serine (S) at position 56, wherein the numbering is relative to SEQ ID NO: 37. In some embodiments, the V_{H} region and the V_{L} region comprise the sequences set forth in SEQ ID NO: 80 and 39, respectively.

In some embodiments, the V_{H} region comprises the CDR-H1, the CDR-H2, and the CDR-H3 contained within SEQ ID NO: 82, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOS: 19, 112 and 21, respectively, and the V_{L} region comprises the CDR-L1, the CDR-L2, and the CDR-L3 comprising the sequence set forth in SEQ ID NOS: 24, 25 and 26, respectively. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 82, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the VH comprises amino acid residues a glutamic acid (E) at position 16, alanine (A) at position 55, aspartic acid (D) at position 58, serine (S) at position 76, tyrosine (Y) at position 80, leucine (L) at position 112, and aspartic acid (D) at position 113, wherein the numbering is relative to SEQ ID NO: 36, and the VL region comprises serine (S) at position 56, wherein the numbering is relative to SEQ ID NO: 37. In some embodiments, the V_{H}region and the V_{L} region comprise the sequences set forth in SEQ ID NO: 82 and 39, respectively.

In some embodiments, the V_{H} region comprises the CDR-H1, the CDR-H2, and the CDR-H3 contained within SEQ ID NO: 84, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOS: 19, 110 and 21, respectively, and the V_{L} region comprises the CDR-L1, the CDR-L2, and the CDR-L3 comprising the sequence set forth in SEQ ID NOS: 24, 25 and 26, respectively. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 84, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the VH comprises amino acid residues a glycine (G) at position 16, alanine (A) at position 55, serine (S) at position 76, tyrosine (Y) at position 80, leucine (L) at position 112, and aspartic acid (D) at position 113, wherein the numbering is relative to SEQ ID NO: 36, and the VL region comprises serine (S) at position 56, wherein the numbering is relative to SEQ ID NO: 37. In some embodiments, the V_{H} region and the V_{L} region comprise the sequences set forth in SEQ ID NO: 84 and 39, respectively.

In some embodiments, the V_{H} region comprises the CDR-H1, the CDR-H2, and the CDR-H3 contained within SEQ ID NO: 86, and the V_{L} region comprises the CDR-L1, the CDR-L2, and the CDR-L3 contained within SEQ ID NO: 39. In some embodiments, the V_{H} region comprises the CDR-H1, the CDR-H2, and the CDR-H3 comprising the sequence set forth in SEQ ID NOS: 19, 110 and 21, respectively, and the V_{L} region comprises the CDR-L1, the CDR-L2, and the CDR-L3 comprising the sequence set forth in SEQ ID NOS: 24, 25 and 26, respectively. In some embodiments, the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 86, and the V_{L} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39. In some embodiments, the VH comprises amino acid residues a glutamic acid (E) at position 16, alanine (A) at position 55, glutamic acid (E) at position 76, tyrosine (Y) at position 80, leucine (L) at position 112, and aspartic acid (D) at position 113, wherein the numbering is relative to SEQ ID NO: 36, and the VL region comprises serine (S) at position 56, wherein the numbering is relative to SEQ ID NO: 37. In some embodiments, the V_{H} region and the V_{L} region comprise the sequences set forth in SEQ ID NO: 86 and 39, respectively.

In some embodiments, the V_{H} region comprises the CDR-H1, the CDR-H2, and the CDR-H3 contained within SEQ ID NO: 159, and the V_{L} region comprises the CDR-L1, the CDR-L2, and the CDR-L3 contained within SEQ ID NO: 43. In some embodiments, the V_{H} region comprises the CDR-H1, the CDR-H2, and the CDR-H3 comprising the sequence set forth in SEQ ID NOS: 19, 20 and 113, respectively, and the V_{L} region comprises the CDR-L1, the CDR-L2, and the CDR-L3 comprising the sequence set forth in SEQ ID NOS: 24, 25 and 115, respectively. In some embodiments, the V_{H} region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 159, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43. In some embodiments, the VH comprises amino acid residues a glycine (G) at position 16, alanine (A) at position 55, threonine (T) at position 58, tyrosine (Y) at position 80, glycine (G) at position 106, and histidine (H) at position 107, wherein the numbering is relative to SEQ ID NO: 36, and the VL region comprises serine (S) at position 56 and asparagine (N) at position 92, wherein the numbering is relative to SEQ ID NO: 37. In some embodiments, the V_{H} region and the V_{L} region comprise the sequences set forth in SEQ ID NO: 159 and 43, respectively.

In some embodiments, the antibody is an antigen-binding fragment containing any of the above V_{H} and V_{L} regions. In some embodiments, the antibody is an scFv containing any of the above VH and VL regions. In some embodiments, the scFv includes a linker joining the two antibody VH and VL region. The linker typically is a peptide linker, e.g., a flexible and/or soluble peptide linker, such as one rich in glycine and serine. In some aspects, the linker rich in glycine and serine (and/or threonine) includes at least 80%, 85%, 90%, 91%, 92%, 93%, 94%, 95%, 96%, 97%, 98%, or 99% glycine, serine, and/or threonine amino acid(s). In some embodiments, the linker includes at least at or about 50%, 55%, 60%, 70%, or 75%, glycine, serine, and/or threonine. In some embodiments, the linker is comprised substantially entirely of glycine, serine, and/or threonine. The linkers generally are between about 5 and about 50 amino acids in length, typically between at or about 10 and at or about 30, e.g., 10, 11, 12, 13, 14, 15, 16, 17, 18, 19, 20, 21, 22, 23, 24, 25, 26, 27, 28, 29, or 30, and in some examples between 10 and 25 amino acids in length. Exemplary linkers include linkers having various numbers of repeats of the sequence GGGGS (4GS; SEQ ID NO: 160) or GGGS (3GS; SEQ ID NO: 217), such as between 2, 3, 4 and 5 repeats of such a sequence. Exemplary linkers include those having the sequence set forth in SEQ ID NO: 162 (GGGGSGGGGSGGGGS). Exemplary linkers further include those having the sequence set forth in SEQ ID NO: 218 (GSTSGSGKPGSGEGSTKG). Exemplary linkers further include those having or consisting of the sequence set forth in SEQ ID NO: 219 (SRGGGGSGGGGSGGGGSLEMA). An exemplary linker includes those having or consisting of the sequence set forth in SEQ ID NO: 220 (GSRGGGGSGGGGSGGGGSLEMA). In some embodiments, the V_{H} region may be amino terminal to the VL region. In some embodiments, the VH region may be carboxy terminal to the V_{L} region. In particular embodiments, the fragment, e.g., scFv, may include a V_{H} region or portion thereof, followed by the linker, followed by a V_{L} region or portion thereof. In other embodiments, the fragment, e.g., the scFv, may include the VL region or portion thereof, followed by the linker, followed by the VH region or portion thereof. In some embodiments, the scFv is a disulfide-stabilized Fv (ds-scFv) in which the VH and VL contain a first and second engineered cysteine residue, respectively, that form a disulfide bond. In some embodiments, the first engineered cysteine residue is at position 44 (e.g, G44C amino acid substitution) in the VH, wherein the numbering is relative to SEQ ID NO: 36, and the second engineered cysteine residue is at position 100 (e.g., Q100C amino acid substitution) in the VL, wherein the numbering is relative to SEQ ID NO: 37.

In some such embodiments, the antibody is a full-length antibody that also contains a heavy chain and light chain constant region. In some embodiments, the antibody comprises a constant region from an IgG1 or IgG2. In some embodiments, the constant region is a human IgG1 heavy chain constant region. In some embodiments, the antibody includes at least a portion of a hinge region or a variant thereof. In some embodiments, the antibody includes a CH2 and/or CH3 domain, such as an Fc region. In some embodiments, the Fc region is an Fc region of a human IgG, such as an IgG1 or IgG4. In some embodiments, the heavy chain constant portion is derived from an IgG1 immunoglobulin constant chain. In some embodiments, the light chain CL is from a kappa or lambda light chain sequence.

In some embodiments, the constant light chain is a λ (lambda) light chain. In some embodiments, the constant light (CL) is set forth by the sequence in SEQ ID NO: 224 or a sequence having at least at or about 85%, 86%, 87%, 88%, 89%, 90%, 91%, 92%, 93%, 94%, 95%, 96%, 97%, 98% or 99% identity to SEQ ID NO:224.

In some embodiments, the constant light chain is a κ (kappa) light chain. In some embodiments, the constant light chain (CL) is set forth by the sequence in SEQ ID NO: 171 or a sequence having at least at or about 85%, 86%, 87%, 88%, 89%, 90%, 91%, 92%, 93%, 94%, 95%, 96%, 97%, 98% or 99% identity to SEQ ID NO:171.

In some embodiments, the constant light chain is a κ (kappa) light chain. In some embodiments, the constant light chain (CL) is set forth by the sequence in SEQ ID NO: 225 or a sequence having at least at or about 85%, 86%, 87%, 88%, 89%, 90%, 91%, 92%, 93%, 94%, 95%, 96%, 97%, 98% or 99% identity to SEQ ID NO: 225.

In some embodiments, the antibody is an antigen-binding fragment that further comprises at least a portion of an immunoglobulin constant region of the heavy chain and at least a portion of an immunoglobulin constant region of the light chain. In some embodiments, the CD3 binding domain is an anti-CD3 antibody that is a Fab containing any of the above V_{H} and V_{L} regions. In some embodiments, the Fab contains a heavy chain comprising any one of the above VH regions and an immunoglobulin constant heavy chain domain 1 (CH1 domain) and a light chain comprising any one of the above VL regions and an immunoglobulin constant light chain (CL).

In some embodiments, the CH1 is from an IgG1 antibody. In some embodiments, the CH1 is set forth by the sequence in SEQ ID NO: 221 or a sequence having at least at or about 85%, 86%, 87%, 88%, 89%, 90%, 91%, 92%, 93%, 94%, 95%, 96%, 97%, 98%, 99% identity to SEQ ID NO:221. In some embodiments, the CH1 is set forth by the sequence in SEQ ID NO: 223 or a sequence having at least at or about 85%, 86%, 87%, 88%, 89%, 90%, 91%, 92%, 93%, 94%, 95%, 96%, 97%, 98% or 99% identity to SEQ ID NO:223. In some embodiments, the CH1 is from an IgG1 antibody. In some embodiments, the CH1 is set forth by the sequence in SEQ ID NO: 169 or a sequence having at least at or about 85%, 86%, 87%, 88%, 89%, 90%, 91%, 92%, 93%, 94%, 95%, 96%, 97%, 98% or 99% identity to SEQ ID NO:169.

In some embodiments, the CH1 is from an IgG2 antibody. In some embodiments, the CH1 is set forth by the sequence in SEQ ID NO: 170 or a sequence having at least at or about 85%, 86%, 87%, 88%, 89%, 90%, 91%, 92%, 93%, 94%, 95%, 96%, 97%, 98% or 99% identity to SEQ ID NO:170.

In some embodiments, the CL is a kappa constant domain. In some embodiments, the constant light chain (CL) is set forth by the sequence in SEQ ID NO: 171 or a sequence having at least at or about 85%, 86%, 87%, 88%, 89%, 90%, 91%, 92%, 93%, 94%, 95%, 96%, 97%, 98% or 99% identity to SEQ ID NO:171. In some embodiments, the constant light chain (CL) is set forth by the sequence in SEQ ID NO: 225 or a sequence having at least at or about 85%, 86%, 87%, 88%, 89%, 90%, 91%, 92%, 93%, 94%, 95%, 96%, 97%, 98% or 99% identity to SEQ ID NO: 225.

In some embodiments, the CL is a lambda constant domain. In some embodiments, the constant light (CL) is set forth by the sequence in SEQ ID NO: 224 or a sequence having at least at or about 85%, 86%, 87%, 88%, 89%, 90%, 91%, 92%, 93%, 94%, 95%, 96%, 97%, 98% or 99% identity to SEQ ID NO: 224.

In some embodiments, the CD3 binding domain is an anti-CD3 antibody that is a Fab composed of a heavy chain comprising any one of the above V_{H} regions and an immunoglobulin constant heavy chain domain 1 (CH1 domain) set forth in SEQ ID NO: 169, and a light chain comprising any one of the above V_{L} regions and an immunoglobulin constant light chain (CL) set forth in SEQ ID NO: 171. In some embodiments, the CD3 binding domain is a Fab comprising a heavy chain comprising a VH set forth by the sequence of SEQ ID NO:68 and a CH1 comprising the sequence set forth in SEQ ID NO:169, and a light chain comprising a VL set forth by the sequence of SEQ ID NO: 39 and a CL comprising the sequence set forth in SEQ ID NO: 171. In some embodiments, the CD3 binding domain is a Fab comprising a heavy chain comprising a VH set forth by the sequence of SEQ ID NO:70 and a CH1 comprising the sequence set forth in SEQ ID NO:169, and a light chain comprising a VL set forth by the sequence of SEQ ID NO: 43 and a CL comprising the sequence set forth in SEQ ID NO: 171.

### 2. Multimerizing Domain (e.g., Fc Domain)

Bispecific antigen-binding molecules of the present invention will typically comprise two multimerizing domains, e.g., two Fc domains that are each individually part of a separate antibody heavy chain. The first and second multimerizing domains may be of the same IgG isotype such as, e.g., IgG1/IgG1, IgG2/IgG2, IgG4/IgG4. Alternatively, the first and second multimerizing domains may be of different IgG isotypes such as, e.g., IgG1/IgG2, IgG1/IgG4, IgG2/IgG4, etc.

Any bispecific antibody format or technology may be used to make the bispecific antigen-binding molecules of the present invention. For example, an antibody or antigen-binding fragment thereof having a first antigen binding specificity can be functionally linked (e.g., by chemical coupling, genetic fusion, noncovalent association or otherwise) to one or more other molecular entities, such as another antibody or antibody fragment having a second antigen-binding specificity to produce a bispecific antigen-binding molecule. Specific exemplary bispecific formats that can be used in the context of the present invention include, without limitation, e.g., scFv-based or diabody bispecific formats, IgG-scFv fusions, dual variable domain (DVD)-Ig, Quadroma, knobs-into-holes, common light chain (e.g., common light chain with knobs-into-holes,etc.), CrossMab, CrossFab, (SEED) body, leucine zipper, Duobody, IgG1/IgG2, dual acting Fab (DAF)-IgG, and Mabe bispecific formats (See, for example, Spiess et al. (2015) Mol Immunol 67(2 Pt A):95-106 and Brinkmann et al. (2017) MAbs 9(2):182-212, and the references cited therein, for a review of the foregoing formats).

In some embodiments, the bispecific antibody is a full-length antibody, such as a full length IgG1 antibody. In some embodiments, the full-length IgG1 antibody is a full length IgG1, λ (lambda), κ (kappa) antibody or IgG1, κ (kappa), κ (kappa) antibody. In some embodiments, the full-length antibody includes an Fc region, e.g. composed of at least a hinge region, a CH2 domain, and a CH3 domain.

In the context of bispecific antigen-binding molecules of the present invention, the multimerizing domains, e.g., Fc domains, may comprise one or more amino acid changes (e.g., insertions, deletions or substitutions) as compared to the wild-type, naturally occurring version of the Fc domain.

In some embodiments, the bispecific antibody is a dimer formed by polypeptides, each containing an Fc region. In some embodiments, the Fc region is formed by Fc domains that are mutated or modified to promote heterodimerization in which different polypeptides can be dimerized to yield a heterodimer. Thus, in some embodiments, the dimer is a heterodimer in which two polypeptide chains of the bispecific polypeptide construct are different. Exemplary modifications to promote heterodimerization are known, including any as described below.

In one aspect, a bispecific antibody provided herein is composed of a heterodimeric Fc region that facilitates interactions of two different heavy chain polypeptides in which each heavy chain polypeptide contains at least one different binding arm of the bispecific antibody. In some embodiments, the bispecific antibody includes a first heavy chain containing at least one anti-CD3 antibody or antigen-binding fragment and a first polypeptide chain of the heterodimeric Fc and a second heavy chain containing an anti-CD3 antibody or antigen-binding fragment and a second polypeptide chain of the heterodimeric Fc. In some embodiments the first polypeptide chain of the heterodimeric Fc-region comprises a first CH3 region, and the second polypeptide chain of the heterodimeric Fc region comprises a second CH3 region, wherein the sequences of the first and second CH3 regions are different and are such that the heterodimeric interaction between said first and second CH3 regions is stronger than each of the homodimeric interactions of said first and second CH3 regions.

In some embodiments, each polypeptide of an immunoglobulin Fc region comprises a human IgG1 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 226. In some embodiments, each polypeptide of an immunoglobulin Fc region comprises a human IgG1 polypeptide sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 227.

In some embodiments, the wild-type constant heavy chain is a wild-type IgG1 constant heavy chain a sequence that is at least 50%, 60%, 65%, 70%, 75%, 80%, 85%, 90%, 91%, 92%, 93%, 94%, 95%, 96%, 97%, 98%, or 99% identical to the amino acid sequence of SEQ ID NO: 253. In some embodiments, the wild-type constant heavy chain comprises the amino acid sequence set forth in SEQ ID NO: 253.

In the context of the provided disclosure the following notations are, unless otherwise indicated, used to describe a mutation; i) substitution of an amino acid in a given position is written as e.g. T350V which means a substitution of a Threonine in position 350 with a Valine; and ii) for specific variants the specific three or one letter codes are used, including the codes Xaa and X to indicate any amino acid residue. Thus, the substitution of Threonine with Valine in position 350 is designated as: T350V.

In some embodiments, an IgG1 Fc polypeptide or a variant thereof such as any described below can be made in a G1m1 or G1 m3 allotype. In some embodiments, the Fc region can contain amino acids of the human G1m1 allotype, such as residues containing Asp (D) and Leu (L) at positions 356 and 358, e.g. as set forth in SEQ ID NO:227. In some cases, an Fc polypeptide can contain amino acid substitutions E356D and M358L to reconstitute residues of allotype G1m1. In other embodiments, the Fc region can contain amino acids of the human G1 m3 allotype, such as residues Glu (E) and Met (M) at positions 356 and 358 by EU numbering, e.g. as set forth in SEQ ID NO: 226. In some cases, an Fc polypeptide can contain amino acid substitutions D356E and L358M to reconstitute residues of allotype G1 m3.

In some embodiments, a bispecific antibody is formatted with an Fc region that lacks a C-terminal Lys (K) residue. In some embodiments, the human IgG1 Fc region lacks Lys447 (EU index of Kabat et al 1991 Sequences of Proteins of Immunological Interest).

In particular embodiments of bispecific antibodies provided herein, the human IgG Fc region is modified to induce heterodimerization. Various methods are known for promoting heterodimerization of complementary Fc polypeptides, see e.g. Ridgway et al, Protein Eng. 9:617-621 (1996); Merchant et al, Nat. Biotechnol. 16(7): 677-81 (1998); Moore et al. (2011) MAbs, 3:546-57; Von Kreudenstein et al. MAbs, (2013) 5:646-54; Gunasekaran et al. (2010) J. Biol. Chem., 285:19637-46; Leaver-Fay et al. (2016) Structure, 24:641-51; Ha et al. (2016) Frontiers in Immunology, 7:1; Davis et al. (2010) Protein Eng Des Sel, 23:195-202; published international PCT Appl. No. WO 1998/050431, WO 2009/089004, WO2011143545 WO 2014/067011, WO 2012/058768, WO2018027025; published U.S. patent Appl. No. US20140363426, US20150307628, US20180016354, US20150239991; and U.S. patent Nos. U.S. Pat. Nos. 5,731,168, 7,183,076, 9,701,759, 9,605,084, and 9,650,446. Methods to promote heterodimerization of Fc chains include mutagenesis of the Fc region, such as by including a set of “knob-into-hole” mutations or including mutations to effect electrostatic steering of the Fc to favor attractive interactions among different polypeptide chains. For example, in some embodiments, the Fc polypeptides of a heterodimer includes a mutation to alter charge polarity across the Fc dimer interface such that co-expression of electrostatically matched Fc chains support favorable attractive interactions thereby promoting desired Fc heterodimer formation, whereas unfavorable repulsive charge interactions suppress unwanted Fc homodimer formation (Guneskaran et al. (2010) JBC, 285: 19637-19646). When co-expressed in a cell, association between the chains is possible but the chains do not substantially self-associate due to charge repulsion. Other strategies for generating a heterodimeric Fc include mixing human IgG and IgA CH3 domain segments to create a complementary CH3 heterodimer, which is referred to as a SEED Fc.

Methods and variants for heterodimerization also include those described in published international PCT App. WO2014/145806, including “knobs and holes” mutations (also called “skew” variants), mutations that relate to “electrostatic steering” or “charge pairs,” and pI variants. Heterodimeric variants also include any as described in U.S. published Appl. No. US2012/0149876 or US2018/011883.

In some embodiments, modifications include introduction of a protuberance (knob) into a first Fc polypeptide and a cavity (hole) into a second Fc polypeptide such that the protuberance is positionable in the cavity to promote complexing of the first and second Fc-containing polypeptides. Amino acids targeted for replacement and/or modification to create protuberances or cavities in a polypeptide are typically interface amino acids that interact or contact with one or more amino acids in the interface of a second polypeptide. In some embodiments, modifications include a knob-into-hole modification and disulfide bond pairing mutation.

In some embodiments, a first Fc polypeptide that is modified to contain protuberance (knob) amino acids include replacement of a native or original amino acid with an amino acid that has at least one side chain which projects from the interface of the first Fc polypeptide and is therefore positionable in a compensatory cavity (hole) in an adjacent interface of a second polypeptide. Most often, the replacement amino acid is one which has a larger side chain volume than the original amino acid residue. One of skill in the art knows how to determine and/or assess the properties of amino acid residues to identify those that are ideal replacement amino acids to create a protuberance. In some embodiments, the replacement residues for the formation of a protuberance are naturally occurring amino acid residues and include, for example, arginine (R), phenylalanine (F), tyrosine (Y), or tryptophan (W). In some examples, the original residue identified for replacement is an amino acid residue that has a small side chain such as, for example, alanine, asparagine, aspartic acid, glycine, serine, threonine, or valine.

In some embodiments, a second Fc polypeptide that is modified to contain a cavity (hole) is one that includes replacement of a native or original amino acid with an amino acid that has at least one side chain that is recessed from the interface of the second polypeptide and thus is able to accommodate a corresponding protuberance from the interface of a first polypeptide. Most often, the replacement amino acid is one which has a smaller side chain volume than the original amino acid residue. One of skill in the art knows how to determine and/or assess the properties of amino acid residues to identify those that are ideal replacement residues for the formation of a cavity. Generally, the replacement residues for the formation of a cavity are naturally occurring amino acids and include, for example, alanine (A), serine (S), threonine (T) and valine (V). In some examples, the original amino acid identified for replacement is an amino acid that has a large side chain such as, for example, tyrosine, arginine, phenylalanine, or tryptophan.

The CH3 interface of human IgG1, for example, involves sixteen residues on each domain located on four anti-parallel β-strands which buries 1090 Å2 from each surface (see e.g., Deisenhofer et al. (1981) Biochemistry, 20:2361-2370; Miller et al., (1990) J Mol. Biol., 216, 965-973; Ridgway et al., (1996) Prot. Engin., 9: 617-621; U.S. Pat. No. 5,731,168). Modifications of a CH3 domain to create protuberances or cavities are described, for example, in U.S. Pat. No. 5,731,168; International Patent Applications WO98/50431 and WO 2005/063816; and Ridgway et al., (1996) Prot. Engin., 9: 617-621. In some examples, modifications of a CH3 domain to create protuberances or cavities are typically targeted to residues located on the two central anti-parallel β-strands. The aim is to minimize the risk that the protuberances which are created can be accommodated by protruding into the surrounding solvent rather than being accommodated by a compensatory cavity in the partner CH3 domain.

In some embodiments, to promote heterodimerization both polypeptides of the Fc heterodimer contain paired or complementary amino acid modifications. Exemplary paired amino acid modification of polypeptides of an Fc fusion are set forth in Table 2.

TABLE 2
Paired amino acids of Heterodimeric Fc

| First Fc polypeptide | Second Fc Polypeptide |
|---|---|
| T366W | T366S/L368W/Y407V |
| T366W/S354C | T366S/L368A/Y407V/Y349C |
| S364H/F405A | Y349T/Y349F |
| T350V/L351Y/F405A/Y407V | T350V/T366L/K392L/T394W |
| K360D/D399M/Y407A | E345R/Q347R/T366V/K409V |
| K409D/K392D | D399K/E356K |
| K360E/K409W | Q347R/D399V/F405T |
| L360E/K409W/Y349C | Q347R/399V/F405T/S354C |
| K370E/K409W | E357N/D399V/F405T |

In general, the Fc region is responsible for effector functions, such as complement-dependent cytotoxicity (CDC) and antibody-dependent cell cytotoxicity (ADCC), in addition to the antigen-binding capacity, which is the main function of immunoglobulins. Additionally, the FcRn sequence present in the Fc region plays the role of regulating the IgG level in serum by increasing the in vivo half-life by conjugation to an in vivo FcRn receptor. In some embodiments, such functions can be altered, such as reduced or enhanced, in an Fc for use with the provided multispecific polypeptide constructs.

In some embodiments, the Fc region includes an Fc polypeptide that is mutated or modified to alter one or more effector functions. Various examples of mutations to Fc polypeptides to alter, such as reduce, effector function are known, including any as described below. In some embodiments, reference to amino acid substitutions in an Fc region is by EU numbering by Kabat (also called Kabat numbering) unless described with reference to a specific SEQ ID NO. EU numbering is known and is according to the most recently updated IMGT Scientific Chart (IMGT®, the international ImMunoGeneTics information system®, http://www.imgt.org/IMGTScientificChart/Numbering/Hu_IGHGnber.html (created: 17 May 2001, last updated: 10 Jan. 2013) and the EU index as reported in Kabat, E. A. et al. Sequences of Proteins of Immunological interest. 5th ed. US Department of Health and Human Services, NIH publication No. 91-3242 (1991).

In some embodiments, the human IgG1 Fc region is modified to alter antibody-dependent cellular cytotoxicity (ADCC) and/or complement-dependent cytotoxicity (CDC), e.g., the amino acid modifications described in Natsume et al., 2008 Cancer Res, 68(10): 3863-72; Idusogie et al., 2001 J Immunol, 166(4): 2571-5; Moore et al., 2010 mAbs, 2(2): 181-189; Lazar et al., 2006 PNAS, 103(11): 4005-4010, Shields et al., 2001 JBC, 276(9): 6591-6604; Stavenhagen et al., 2007 Cancer Res, 67(18): 8882-8890; Stavenhagen et al., 2008 Advan. Enzyme Regul., 48: 152-164; Alegre et al, 1992 J Immunol, 148: 3461-3468; Reviewed in Kaneko and Niwa, 2011 Biodrugs, 25(1):1-11; U.S. Pat. Nos. 8,101,720; 9,475,881; and U.S. patent Ser. No. 10/851,164, the contents of each of which are hereby incorporated by reference in their entireties.

In some embodiments, the Fc region, such as the human IgG1 Fc region is modified to enhance ADCC activity or CDC activity. Examples of mutations that enhance ADCC include modification at Ser239 and Ile332, for example Ser239Asp and Ile332Glu (S239D, I332E). Examples of mutations that enhance CDC include modifications at Lys326 and Glu333. In some embodiments, the Fc region is modified at one or both of these positions, for example Lys326Ala and/or Glu333Ala (K326A and E333A) using the Kabat numbering system.

In some embodiments, the Fc region is altered to provide reduced Fc-mediated effector functions, such as via reduced Fc receptor binding, e.g. binding to FcγR binding but generally not FcRn binding. In some embodiments, the human IgG1 Fc region fusion proteins of the present disclosure lack or have reduced Fucose attached to the N-linked glycan-chain at N297. There are numerous ways to prevent fucosylation, including but not limited to production in a FUT8 deficient cell line; addition inhibitors to the mammalian cell culture media, for example Castanospermine; and metabolic engineering of the production cell line. In some embodiments, the human IgG1 Fc region is modified at amino acid Asn297 to prevent glycosylation of the fusion protein, e.g., Asn297Ala (N297A) or Asn297Asp (N297D).

In some embodiments, the Fc region of the fusion protein is altered at one or more of the following positions to reduce Fc receptor binding: Leu 234 (L234), Leu235 (L235), Asp265 (D265), Asp270 (D270), Ser298 (S298), Asn297 (N297), Asn325 (N325) orAla327 (A327). For example, Leu 234Ala (L234A), Leu235Ala (L235A), Leu235Glu (L235E), Gly237Ala (G237A) Asp265Asn (D265N), Ser267Lys (S267K), Asp270Asn (D270N), Arg292Cys (R292C), Asn297Gly (N297G), Ser298Asn (S298N), Asn297Ala (N297A), Val302Cys (V302C), Asn325Glu (N325E), Ala327Ser (A327S) or Pro329Gly (P329G). In some embodiments, the Fc region of the fusion protein is modified at amino acid Leu235 to alter Fc receptor interactions, e.g., Leu235Glu (L235E) or Leu235Ala (L235A). In some embodiments, the Fc region of the fusion protein is modified at amino acid Leu234 to alter Fc receptor interactions, e.g., Leu234Ala (L234A). In some embodiments, the Fc region of the fusion protein is altered at both amino acid 234 and 235, e.g., Leu234Ala and Leu235Ala (L234A/L235A) or Leu234Val and Leu235Ala (L234V/L235A). In particular embodiments, the Fc region contains LALA mutations Leu234Ala and Leu235Ala (L234A/L235A). In some embodiments, modifications within the Fc region reduce binding to Fc-receptor-gamma receptors while have minimal impact on binding to the neonatal Fc receptor (FcRn).

In some embodiments, the Fc comprises amino acid modification of at least one of L234, L235, or D265. In another embodiment, the Fc comprises amino acid modification of at least L234, L235 and D265. In another embodiment, the Fc comprises the amino acid modification L234A, L235A and D265S. In particular aspects, the IgG1 has been modified to comprise or consist of an L234A, an L235A, and a D265S mutation as identified using Kabat numbering to render the Fc effector-less.

In some embodiments, the Fc comprises a P329G mutation in the Fc together with L234A-L235A (LALA) mutations. In particular aspects, the IgG1 has been modified to comprise or consist of an L234A, an L235A, and a P329G mutation as identified using Kabat numbering to render the Fc effector-less. In some embodiments, P329G is able to disrupt the interaction between hIgG and hFcγR, which combined with the effectorless mutations L234A/L235A mutations create a triple mutant L234A/L235A/P329G with no detectable binding to C1q or FcγRs, resulting in abrogated ADCC. In some embodiments, such an Fc disrupts the interaction with Fcγ-receptors (FcγRs) while maintaining the binding to neonatal Fc-receptor (nFcR) and therefore wildtype-antibody-like pharmacokinetics in subjects.

In some embodiments, the bispecific antigen-binding molecules comprising one or more modifications in the Fc domain that results in a modified Fc domain having a modified binding interaction (e.g., enhanced or diminished) between Fc and FcRn. In some embodiments, the human IgG Fc region is modified to enhance FcRn binding. In one embodiment, the bispecific antigen-binding molecule comprises a modification in a CH2 or a CH3 region, wherein the modification increases the affinity of the Fc domain to FcRn in an acidic environment (e.g., in an endosome where pH ranges from about 5.5 to about 6.0). Examples of Fc mutations that enhance binding to FcRn are Met252Tyr, Ser254Thr, Thr256Glu (M252Y, S254T, T256E, respectively) (Kabat numbering, Dall'Acqua et al 2006, J. Biol Chem Vol. 281(33) 23514-23524), Met428Leu and Asn434Ser (M428L, N434S) (Zalevsky et al 2010 Nature Biotech, Vol. 28(2) 157-159) (EU index of Kabat et al 1991 Sequences of Proteins of Immunological Interest). Non-limiting examples of such Fc modifications include, e.g., a modification at position 250 (e.g., E or Q); 250 and 428 (e.g., L or F);252 (e.g., L/Y/F/W or T), 254 (e.g., S or T), and 256 (e.g., S/R/Q/E/D orT); or a modification at position 428 and/or 433 (e.g., L/R/S/P/Q or K) and/or 434 (e.g., H/F or Y); or a modification at position 250 and/or428; or a modification at position 307 or 308 (e.g., 308F, V308F), and434. In some embodiments, the mutated or modified Fc polypeptide includes the following mutations: Met252Tyr and Met428Leu or Met252Tyr and Met428Val (M252Y, M428L, or M252Y, M428V) using the Kabat numbering system. In one embodiment, the modification comprises a 428L (e.g., M428L) and 434S (e.g., N434S) modification; a 428L, 259I (e.g., V259I), and 308F (e.g., V308F) modification; a 433K (e.g., H433K) and a 434 (e.g., 434Y)modification; a 252, 254, and 256 (e.g., 252Y, 254T, and 256E)modification; a 250Q and 428L modification (e.g., T250Q and M428L); and a307 and/or 308 modification (e.g., 308F or 308P).

In some embodiments, the Fc region is mutated in one or more of the following positions to reduce Fc receptor binding: Glu233 (E233), Leu234 (L234), or Leu235 (L235). The one or more mutations can include E233P, L234V and/or L235A.

In some embodiments, the Fc region of the fusion protein is altered at Gly236 to reduce Fc receptor binding. For example, wherein Gly236 is deleted from the fusion protein. In some embodiments, the human IgG1 Fc region is modified at amino acid Gly236 to enhance the interaction with CD32A, e.g., Gly236Ala (G236A).

In particular embodiments, the mutations of the Fc region to reduce Fc effector function, e.g. via reducing Fc receptor binding to FcγR, include mutations from among any of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G or E233P/L234V/L235A/G236del.

In some embodiments, any of the provided Fc regions may include a mutation to eliminate or reduce the effector function of any antibody by mutating P238, e.g. to P238K, such as in an IgG1 or IgG2 Fc. In some embodiments, in addition to the modifications such as mutations described herein to antibodies to enhance their biological activity or reduce effector function, further mutations can be made, e.g., to the CH1, hinge, CH2 or CH3 domain, e.g., to further reduce the effector function, binding to FcγRs, and/or the stability of the antibodies. For example, any of the mutations described herein may be combined with a P238, e.g., P238K, mutation, such as in an IgG1 or IgG2 Fc. In some embodiments, Fc regions with P238K mutation demonstrate essentially no detectable binding signal towards the low affinity FcγRs hCD32a-H131, hCD32a-R131, hCD32b, hCD16a-V158 or hCD16b-NA2 and/or reduced binding affinity and a faster off-rate to the high affinity FcγR CD64 (see e.g., U.S. published patent application No. US20240141059).

In some embodiments, the bispecific antibody contains an: (i) amino acid substitution T366W in the first polypeptide of the heterodimeric Fc and amino acid substitutions T366S, L368A and Y407V in the second polypeptide of the heterodimeric Fc; and/or (ii) amino acid substitution S354C on the first polypeptide of the heterodimeric Fc and amino acid substitution Y349C on the second polypeptide of the heterodimeric Fc.

In some embodiments, the first Fc polypeptide is an IgG1 Fc comprising amino acid substitutions L234A, L235A, D265S, S354C and T366W, and the second Fc polypeptide is an IgG1 Fc comprising amino acid substitutions L234A, L235A, D265S, Y349C, T366S, L368A and Y407V.

In some embodiments, the bispecific antibody contains a heterodimeric Fc region in which the first Fc polypeptide of the heterodimeric Fc has at least 85%, 90%, 95% or 98% sequence identity to the Fc polypeptide set forth in SEQ ID NO:163 and contains the mutations S354C, T366W and L234A, L235E, G237A; and the second Fc polypeptide of the heterodimeric Fe has at least 85%, 90%, 95% or 98% sequence identity to the Fc polypeptide set forth in SEQ ID NO:164 and contains the mutations Y349C, T366S, L368A, Y407V and L234A, L235E, G237A. In some embodiments, the bispecific antibody contains a heterodimeric Fc region in which the first Fc polypeptide of the heterodimeric Fc is set forth in SEQ ID NO:163 and the second Fc polypeptide of the heterodimeric Fc is set forth in SEQ ID NO:164.

In some embodiments, the bispecific antibody contains a heterodimeric Fc region in which the first Fc polypeptide of the heterodimeric Fc has at least 85%, 90%, 95% or 98% sequence identity to the Fc polypeptide set forth in SEQ ID NO:165 and contains the mutations S354C, T366W and L234A, L235A, D265S; and the second Fc polypeptide of the heterodimeric Fc has at least 85%, 90%, 95% or 98% sequence identity to the Fc polypeptide set forth in SEQ ID NO:166 and contains the mutations Y349C, T366S, L368A, Y407V and L234A, L235A, D265S. In some embodiments, the bispecific antibody contains a heterodimeric Fc region in which the first Fc polypeptide of the heterodimeric Fc is set forth in SEQ ID NO:165 and the second Fc polypeptide of the heterodimeric Fc is set forth in SEQ ID NO:166.

In some embodiments, the bispecific antibody contains a heterodimeric Fc region in which the first Fc polypeptide of the heterodimeric Fc has at least 85%, 90%, 95% or 98% sequence identity to the Fc polypeptide set forth in SEQ ID NO: 228 and contains the mutations T350V, L351Y, F405A and Y407V; and the second Fc polypeptide of the heterodimeric Fc has at least 85%, 90%, 95% or 98% sequence identity to the Fc polypeptide set forth in SEQ ID NO:229 and contains the mutations T350V, T366L, K392L and T394W. In some embodiments, the bispecific antibody contains a heterodimeric Fc region in which the first Fc polypeptide is set forth in SEQ ID NO: 228 and the second Fc polypeptide is set forth in SEQ ID NO: 229.

In some embodiments, the bispecific antibody contains a heterodimeric Fc region in which the first Fc polypeptide of the heterodimeric Fc has at least 85%, 90%, 95% or 98% sequence identity to the Fc polypeptide set forth in SEQ ID NO: 230 and contains the mutations T350V, L351Y, F405A and Y407V and the mutations L234A, L235A and D265S; and the second Fc polypeptide of the heterodimeric Fc has at least 85%, 90%, 95% or 98% sequence identity to the Fc polypeptide set forth in SEQ ID NO: 231 and contains the mutations T350V, T366L, K392L and T394W and the mutations L234A, L235A and D265S. In some embodiments, the bispecific antibody contains a heterodimeric Fc region in which the first Fc polypeptide is set forth in SEQ ID NO: 230 and the second Fc polypeptide is set forth in SEQ ID NO: 231.

In some embodiments, the bispecific antibody contains a heterodimeric Fc region in which the first Fc polypeptide of the heterodimeric Fc has at least 85%, 90%, 95% or 98% sequence identity to the Fc polypeptide set forth in SEQ ID NO: 232 and contains the mutations T350V, L351Y, F405A and Y407V and the mutations L234A, L235A and P329G; and the second Fc polypeptide of the heterodimeric Fc region has at least 85%, 90%, 95% or 98% sequence identity to the Fc polypeptide set forth in SEQ ID NO: 233 and contains the mutations T350V, T366L, K392L and T394W and the mutations L234A, L235A and P329G. In some embodiments, the bispecific antibody contains a heterodimeric Fc region in which the first Fc polypeptide is set forth in SEQ ID NO: 232 and the second Fc polypeptide is set forth in SEQ ID NO: 233.

In some embodiments, the bispecific antibody contains a heterodimeric Fc region in which the first Fc polypeptide of the heterodimeric Fc has at least 85%, 90%, 95% or 98% sequence identity to the Fc polypeptide set forth in SEQ ID NO:167 and contains the mutations S354C, T366W, P238K, and optionally also D356E, and L358M; and the second Fc polypeptide of the heterodimeric Fc has at least 85%, 90%, 95% or 98% sequence identity to the Fc polypeptide set forth in SEQ ID NO:168 and contains the mutations Y349C, T366S, L368A, Y407V, P238K, and optionally also D356E, and L358M. In some embodiments, the bispecific antibody contains a heterodimeric Fc region in which the first Fc polypeptide of the heterodimeric Fc is set forth in SEQ ID NO:167 and the second Fc polypeptide of the heterodimeric Fc is set forth in SEQ ID NO:168.

### 3. Exemplary Bispecific Binding Molecules

In some embodiments, the provided binding molecules are bispecific antibodies that include two antigen binding sites, namely one antigen binding site for CD19 and one antigen binding site for CD3. In some embodiments, provided bispecific binding molecules are bivalent bispecific antibodies that are monovalent for binding to CD19 and monovalent for binding to CD3.

In some embodiments, there is provided a bispecific antibody comprising a first binding region that binds to CD19 and a second binding region that binds to CD3. In some embodiments, the first binding domain and the second binding domain are each N-terminal to the Fc region.

In some embodiments, the first binding domain comprises the VH set forth in any one of SEQ ID NOs: 12 or 88 and the VL set forth in any one of SEQ ID NO: 17 or 89; and the second binding domain comprises the VH set forth in any one of SEQ ID NOs: 22, 68, 72, or 70 and the VL set forth in any one of SEQ ID NO: 27, 39, 73, or 43.

In some embodiments, the first binding domain comprises the VH set forth in SEQ ID NO: 12 and the VL set forth in SEQ ID NO: 17; and the second binding domain comprises the VH set forth in SEQ ID NO: 22 and the VL set forth in SEQ ID NO: 27.

In some embodiments, the first binding domain comprises the VH set forth in SEQ ID NO: 12 and the VL set forth in SEQ ID NO: 17; and the second binding domain comprises the VH set forth in SEQ ID NO: 68 and the VL set forth in SEQ ID NO: 39.

In some embodiments, the first binding domain comprises the VH set forth in SEQ ID NO: 12 and the VL set forth in SEQ ID NO: 17; and the CD3 binding domain comprises the VH set forth in SEQ ID NO: 72 and the VL set forth in SEQ ID NO: 73.

In some embodiments, the first binding domain comprises the VH set forth in SEQ ID NO: 12 and the VL set forth in SEQ ID NO: 17; and the second binding domain comprises the VH set forth in SEQ ID NO: 70 and the VL set forth in SEQ ID NO: 43.

In some embodiments, the first binding domain comprises the VH set forth in SEQ ID NO: 88 and the VL set forth in SEQ ID NO: 89; and the second binding domain comprises the VH set forth in SEQ ID NO: 22 and the VL set forth in SEQ ID NO: 27.

In some embodiments, the first binding domain comprises the VH set forth in SEQ ID NO: 88 and the VL set forth in SEQ ID NO: 89; and the second binding domain comprises the VH set forth in SEQ ID NO: 68 and the VL set forth in SEQ ID NO: 39.

In some embodiments, the first binding domain comprises the VH set forth in SEQ ID NO: 88 and the VL set forth in SEQ ID NO: 89; and the second binding domain comprises the VH set forth in SEQ ID NO: 72 and the VL set forth in SEQ ID NO: 73.

In some embodiments, the first binding domain comprises the VH set forth in SEQ ID NO: 88 and the VL set forth in SEQ ID NO: 89; and the second binding domain comprises the VH set forth in SEQ ID NO: 70 and the VL set forth in SEQ ID NO: 43.

In some embodiments, the bispecific antibody provided herein is composed of one Fab fragment of an antibody specifically binding to CD3 (further named also as “CD3-Fab”), and one scFv fragment of an antibody specifically binding to CD19 (further named also as “CD19-scFv”) and a Fc region. In some embodiments, the CD19-scFv and the heavy chain of the CD3-Fab are linked via their C-termini to the hinge region of said Fc region. In some embodiments, the heavy chain of the CD3-Fab is associated with a light chain (LC) to form the second binding region. In some embodiments, the bispecific antibody is composed of (a) a first heavy chain (HC1) comprising a first amino acid sequence having the formula: a VH-VL of a CD19 scFv and a second amino acid sequence having the following formula: hinge-CH2-CH3 of an Fc region (e.g., a polypeptide of a heterodimeric Fc region, such as a knob or hole polypeptide), and (b) a second heavy chain (HC2) comprising a first amino acid sequence having the formula: VH1-CH1 of a CD3 heavy chain Fab (e.g., as described herein) and a second amino acid sequence having the formula: hinge-CH2-CH3 of an Fc region (e.g., a polypeptide of a heterodimeric Fc region, such as the other of the knob or a hole of an Fc polypeptide). In some embodiments, the bispecific antibody is further composed of a light chain (LC; VL-CL). In some embodiments, the VH1-CH1 of HC2 associates with the LC to form the second binding region. In some embodiments, the first binding domain and the second binding domain are together present on two polypeptide chains.

In some embodiments, the bispecific antibody, comprises: (a) a first heavy chain comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and (b) a second heavy chain polypeptide comprising from N- to C-terminal order: a Fab comprising a VH region comprising the sequence set forth in SEQ ID NO: 68 and an IgG1 CH1 of SEQ ID NO: 169, and a Fc region comprising the sequence set forth in SEQ ID NO:165; and (c) a light chain comprises a VL region comprising the sequence set forth in SEQ ID NO: 39 and a CL comprising the sequence set forth in SEQ ID NO: 171. In some embodiments, the first heavy chain polypeptide comprises from N-to C-terminal order the scFv comprising the sequence set forth in SEQ ID NO:245 and the Fc region comprising the sequence set forth in SEQ ID NO:166. In some embodiments, the first heavy chain (HC1) comprises the amino acid sequence set forth in SEQ ID NO: 174 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 174, a second heavy chain (HC2) comprises the amino acid sequence set forth in SEQ ID NO: 175 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 175, and the light chain (LC) comprises the amino acid sequence set forth in SEQ ID NO: 176 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 176. In some embodiments, the HC1 comprises the sequence set forth in SEQ ID NO: 174, the HC2 comprises the sequence set forth in SEQ ID NO: 175 and the LC comprises the sequence set forth in SEQ ID NO: 176.

In some embodiments, the bispecific antibody, comprises: (a) a first heavy chain comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and (b) a second heavy chain polypeptide comprising from N- to C-terminal order: a Fab comprising a VH region comprising the sequence set forth in SEQ ID NO: 70 and an IgG1 CH1 of SEQ ID NO: 169, and a Fc region comprising the sequence set forth in SEQ ID NO:165; and (c) a light chain comprises a VL region comprising the sequence set forth in SEQ ID NO: 43 and a CL comprising the sequence set forth in SEQ ID NO: 171. In some embodiments, the first heavy chain polypeptide comprises from N-to C-terminal order the scFv comprising the sequence set forth in SEQ ID NO:245 and the Fc region comprising the sequence set forth in SEQ ID NO:166. In some embodiments, the first heavy chain (HC1) comprises the amino acid sequence set forth in SEQ ID NO: 179 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 179, a second heavy chain (HC2) comprises the amino acid sequence set forth in SEQ ID NO: 180 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 180, and the light chain (LC) comprises the amino acid sequence set forth in SEQ ID NO: 181 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 181. In some embodiments, the HC1 comprises the sequence set forth in SEQ ID NO: 179, the HC2 comprises the sequence set forth in SEQ ID NO: 180 and the LC comprises the sequence set forth in SEQ ID NO: 181.

In some embodiments, the bispecific antibody, comprises: (a) a first heavy chain comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and (b) a second heavy chain polypeptide comprising from N- to C-terminal order: a Fab comprising a VH region comprising the sequence set forth in SEQ ID NO: 68 and an IgG1 CH1 of SEQ ID NO: 169, and a Fc region comprising the sequence set forth in SEQ ID NO:165; and (c) a light chain comprises a VL region comprising the sequence set forth in SEQ ID NO: 39 and a CL comprising the sequence set forth in SEQ ID NO: 171. In some embodiments, the first heavy chain polypeptide comprises from N-to C-terminal order the scFv comprising the sequence set forth in SEQ ID NO:248 and the Fc region comprising the sequence set forth in SEQ ID NO:166. In some embodiments, the first heavy chain (HC1) comprises the amino acid sequence set forth in SEQ ID NO: 184 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 184, a second heavy chain (HC2) comprises the amino acid sequence set forth in SEQ ID NO: 185 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 185, and the light chain (LC) comprises the amino acid sequence set forth in SEQ ID NO: 186 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 186. In some embodiments, the HC1 comprises the sequence set forth in SEQ ID NO: 184, the HC2 comprises the sequence set forth in SEQ ID NO: 185 and the LC comprises the sequence set forth in SEQ ID NO: 186.

In some embodiments, the bispecific antibody, comprises: (a) a first heavy chain comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and (b) a second heavy chain polypeptide comprising from N- to C-terminal order: a Fab comprising a VH region comprising the sequence set forth in SEQ ID NO: 70 and an IgG1 CH1 of SEQ ID NO: 169, and a Fc region comprising the sequence set forth in SEQ ID NO:165; and (c) a light chain comprises a VL region comprising the sequence set forth in SEQ ID NO: 43 and a CL comprising the sequence set forth in SEQ ID NO: 171. In some embodiments, the first heavy chain polypeptide comprises from N-to C-terminal order the scFv comprising the sequence set forth in SEQ ID NO:248 and the Fc region comprising the sequence set forth in SEQ ID NO:166. In some embodiments, the first heavy chain (HC1) comprises the amino acid sequence set forth in SEQ ID NO: 189 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 189, a second heavy chain (HC2) comprises the amino acid sequence set forth in SEQ ID NO: 190 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 190, and the light chain (LC) comprises the amino acid sequence set forth in SEQ ID NO: 191 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 191. In some embodiments, the HC1 comprises the sequence set forth in SEQ ID NO: 189, the HC2 comprises the sequence set forth in SEQ ID NO: 190 and the LC comprises the sequence set forth in SEQ ID NO: 191.

In some embodiments, the bispecific antibody, comprises: (a) a first heavy chain comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 168; and (b) a second heavy chain polypeptide comprising from N- to C-terminal order: a Fab comprising a VH region comprising the sequence set forth in SEQ ID NO: 68 and an IgG1 CH1 of SEQ ID NO: 170, and a Fc region comprising the sequence set forth in SEQ ID NO:167; and (c) a light chain comprises a VL region comprising the sequence set forth in SEQ ID NO: 39 and a CL comprising the sequence set forth in SEQ ID NO: 171. In some embodiments, the first heavy chain polypeptide comprises from N-to C-terminal order the scFv comprising the sequence set forth in SEQ ID NO:245 and the Fc region comprising the sequence set forth in SEQ ID NO:168. In some embodiments, the first heavy chain (HC1) comprises the amino acid sequence set forth in SEQ ID NO: 234 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 234, a second heavy chain (HC2) comprises the amino acid sequence set forth in SEQ ID NO: 235 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 235, and the light chain (LC) comprises the amino acid sequence set forth in SEQ ID NO: 236 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 236. In some embodiments, the HC1 comprises the sequence set forth in SEQ ID NO: 234, the HC2 comprises the sequence set forth in SEQ ID NO: 235 and the LC comprises the sequence set forth in SEQ ID NO: 236.

In some embodiments, the bispecific antibody, comprises: (a) a first heavy chain comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 168; and (b) a second heavy chain polypeptide comprising from N- to C-terminal order: a Fab comprising a VH region comprising the sequence set forth in SEQ ID NO: 70 and an IgG1 CH1 of SEQ ID NO: 170, and a Fc region comprising the sequence set forth in SEQ ID NO:167; and (c) a light chain comprises a VL region comprising the sequence set forth in SEQ ID NO: 43 and a CL comprising the sequence set forth in SEQ ID NO: 171. In some embodiments, the first heavy chain polypeptide comprises from N-to C-terminal order the scFv comprising the sequence set forth in SEQ ID NO:245 and the Fc region comprising the sequence set forth in SEQ ID NO:168. In some embodiments, the first heavy chain (HC1) comprises the amino acid sequence set forth in SEQ ID NO: 237 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 237, a second heavy chain (HC2) comprises the amino acid sequence set forth in SEQ ID NO: 238 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 238, and the light chain (LC) comprises the amino acid sequence set forth in SEQ ID NO: 239 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 239. In some embodiments, the HC1 comprises the sequence set forth in SEQ ID NO: 237, the HC2 comprises the sequence set forth in SEQ ID NO: 238 and the LC comprises the sequence set forth in SEQ ID NO: 239.

In some embodiments, there is provided a bispecific antibody comprising a first binding region that binds to CD19 and a second binding region that binds to CD3. In some embodiments, the bispecific antibody provided herein is composed of one scFv fragment of an antibody specifically binding to CD3 (further named also as “CD3-scFv”), and one scFv fragment of an antibody specifically binding to CD19 (further named also as “CD19-scFv”) and a Fc region. In some embodiments, the CD19-scFv and the CD3-scFv are linked via their C-termini to the hinge region of said Fc region. In some embodiments, the bispecific antibody is composed of (a) a first heavy chain (HC1) comprising a first amino acid sequence having the formula: a VH-VL of a CD19 scFv and a second amino acid sequence having the following formula: hinge-CH2-CH3 of an Fc region (e.g., a polypeptide of a heterodimeric Fc region, such as a knob or hole polypeptide), and (b) a second heavy chain (HC2) comprising a first amino acid sequence having the formula: VH-VL of a CD3 scFv (e.g., as described herein) and a second amino acid sequence having the formula: hinge-CH2-CH3 of an Fc region (e.g., a polypeptide of a heterodimeric Fc region, such as the other of the knob or a hole of an Fc polypeptide).

In some embodiments, the bispecific antibody, comprises: (a) a first heavy chain comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and (b) a second heavy chain polypeptide comprising from N- to C-terminal order: a VH region comprising the sequence set forth in SEQ ID NO: 22, a linker of SEQ ID NO: 162, a VL region comprising the sequence set forth in SEQ ID NO: 27, and a Fc region comprising the sequence set forth in SEQ ID NO:165. In some embodiments, the first heavy chain polypeptide comprises from N-to C-terminal order the scFv comprising the sequence set forth in SEQ ID NO:245 and the Fc region comprising the sequence set forth in SEQ ID NO:166; and the second heavy chain polypeptide comprises from N-to C-terminal order the scFv comprising the sequence set forth in SEQ ID NO:246 and the Fc region comprising the sequence set forth in SEQ ID NO: 165. In some embodiments, the first heavy chain (HC1) comprises the amino acid sequence set forth in SEQ ID NO: 172 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 172, a second heavy chain (HC2) comprises the amino acid sequence set forth in SEQ ID NO: 173 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 173. In some embodiments, the HC1 comprises the sequence set forth in SEQ ID NO: 172 and the HC2 comprises the sequence set forth in SEQ ID NO: 173.

In some embodiments, the bispecific antibody, comprises: (a) a first heavy chain comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and (b) a second heavy chain polypeptide comprising from N- to C-terminal order: a VH region comprising the sequence set forth in SEQ ID NO: 72, a linker of SEQ ID NO: 162, a VL region comprising the sequence set forth in SEQ ID NO: 73, and a Fc region comprising the sequence set forth in SEQ ID NO:165. In some embodiments, the first heavy chain polypeptide comprises from N-to C-terminal order the scFv comprising the sequence set forth in SEQ ID NO:245 and the Fc region comprising the sequence set forth in SEQ ID NO:166; and the second heavy chain polypeptide comprises from N-to C-terminal order the scFv comprising the sequence set forth in SEQ ID NO:247 and the Fc region comprising the sequence set forth in SEQ ID NO: 165. In some embodiments, the first heavy chain (HC1) comprises the amino acid sequence set forth in SEQ ID NO: 177 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 177, a second heavy chain (HC2) comprises the amino acid sequence set forth in SEQ ID NO: 178 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 178. In some embodiments, the HC1 comprises the sequence set forth in SEQ ID NO: 177 and the HC2 comprises the sequence set forth in SEQ ID NO: 178.

In some embodiments, the bispecific antibody, comprises: (a) a first heavy chain comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and (b) a second heavy chain polypeptide comprising from N- to C-terminal order: a VH region comprising the sequence set forth in SEQ ID NO: 22, a linker of SEQ ID NO: 162, a VL region comprising the sequence set forth in SEQ ID NO: 27, and a Fc region comprising the sequence set forth in SEQ ID NO:165. In some embodiments, the first heavy chain polypeptide comprises from N-to C-terminal order the scFv comprising the sequence set forth in SEQ ID NO:248 and the Fc region comprising the sequence set forth in SEQ ID NO:166; and the second heavy chain polypeptide comprises from N-to C-terminal order the scFv comprising the sequence set forth in SEQ ID NO:246 and the Fc region comprising the sequence set forth in SEQ ID NO: 165. In some embodiments, the first heavy chain (HC1) comprises the amino acid sequence set forth in SEQ ID NO: 182 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 182, a second heavy chain (HC2) comprises the amino acid sequence set forth in SEQ ID NO: 183 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 183. In some embodiments, the HC1 comprises the sequence set forth in SEQ ID NO: 182 and the HC2 comprises the sequence set forth in SEQ ID NO: 183.

In some embodiments, the bispecific antibody, comprises: (a) a first heavy chain comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and (b) a second heavy chain polypeptide comprising from N- to C-terminal order: a VH region comprising the sequence set forth in SEQ ID NO: 72, a linker of SEQ ID NO: 162, a VL region comprising the sequence set forth in SEQ ID NO: 73, and a Fc region comprising the sequence set forth in SEQ ID NO:165. In some embodiments, the first heavy chain polypeptide comprises from N-to C-terminal order the scFv comprising the sequence set forth in SEQ ID NO:248 and the Fc region comprising the sequence set forth in SEQ ID NO:166; and the second heavy chain polypeptide comprises from N-to C-terminal order the scFv comprising the sequence set forth in SEQ ID NO:247 and the Fc region comprising the sequence set forth in SEQ ID NO: 165. In some embodiments, the first heavy chain (HC1) comprises the amino acid sequence set forth in SEQ ID NO: 187 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 187, a second heavy chain (HC2) comprises the amino acid sequence set forth in SEQ ID NO: 188 or an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 188. In some embodiments, the HC1 comprises the sequence set forth in SEQ ID NO: 187 and the HC2 comprises the sequence set forth in SEQ ID NO: 188.

### C. Polynucleotides, Vectors and Methods of Producing the Binding Molecule

The present disclosure provides a nucleic acid or nucleic acids encoding any one of the CD19 binding molecules, such as CD19 monospecific antibody binding domains and also bispecific binding molecules having a CD19 antibody binding domain specific to CD19 and a CD3 antibody binding domain specific to CD3, including full-length antibodies and antigen-binding fragments of any such molecule as described herein. The polypeptide sequences may be used to determine appropriate nucleic acid sequences encoding the binding domains or binding molecules disclosed thereby. The nucleic acid sequence may be optimized to reflect particular codon “preferences” for various expression systems according to standard methods well known to those of skill in the art.

Among the provided nucleic acid molecules are those that comprise polynucleotides that encode one or more polypeptides (e.g., an immunoglobulin heavy chain or light chain) of a provided antibody binding molecule, such as monospecific or bispecific antibody molecules, including full-length antibodies and functional antigen-binding fragments. In some embodiments, a nucleic acid molecule comprises a polynucleotide that encodes a heavy chain or a light chain of an antibody binding molecule, such as any of the described monospecific or bispecific antibody molecules, including full-length antibodies and functional antigen-binding fragments. In some embodiments, a nucleic acid molecule comprises both a polynucleotide that encodes a heavy chain and a polynucleotide that encodes a light chain of any of the described antibody binding molecules. In some embodiments, a first nucleic acid molecule comprises a first polynucleotide that encodes a heavy chain and a second nucleic acid molecule comprises a second polynucleotide that encodes a light chain. Among the provided nucleic acid molecules are nucleic acid molecules comprising polynucleotides that encode one or more polypeptides of a binding molecule containing any of the provided CD19 binding domains or a bispecific immune cell engager comprising the CD19 binding domain and a CD3 binding domain as described herein.

In some embodiments, the nucleic acid(s) is an isolated nucleic acid(s). The polynucleotides may include those encompassing natural and/or non-naturally occurring nucleotides and bases, e.g., including those with backbone modifications. The terms “nucleic acid molecule”, “nucleic acid”, “sequence of nucleotides”, and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

In some such embodiments, the heavy chain and the light chain are expressed from one nucleic acid molecule, or from two separate nucleic acid molecules, as two separate polypeptides. In some embodiments, such as when an antibody is an scFv, a single polynucleotide encodes a single polypeptide comprising both a heavy chain variable domain fragment and a light chain variable domain fragment linked together.

This disclosure is not intended to be limited with regard to the source of the antibody or the manner in which it is made. Various procedures known within the art may be used for the production of monoclonal antibodies directed against CD3 (See, for example, Antibodies: A Laboratory Manual, Harlow E, and Lane D, 1988, Cold Spring Harbor Laboratory Press, Cold Spring Harbor, NY, incorporated herein by reference). In some embodiments, the CD3 binding domain, and antigen binding fragments thereof, are recombinantly expressed and produced.

In some embodiments, a nucleic acid of the present disclosure may be operably linked to a transcriptional control element, e.g., a promoter, and enhancer, etc. Suitable promoter and enhancer elements are known to those of skill in the art.

In some embodiments, a polynucleotide encoding a heavy chain or light chain of a provided binding molecule, including full-length antibody binding domains or antigen binding fragments or bispecific antibodies as described, comprises a nucleotide sequence that encodes a leader sequence, which, when translated, is located at the N terminus of the heavy chain or light chain. The leader sequence may be the native heavy or light chain leader sequence, or may be another heterologous leader sequence.

Also provided are polynucleotides that have been optimized for codon usage and/or to eliminate splice sites, such as cryptic splice sites. In some embodiments, the polynucleotides are modified to optimize codon usage. In some embodiments, the polynucleotides are codon optimized for expression in a mammalian cell, such as a CHO cell, or a human cell.

Nucleic acid molecules may be constructed using conventional recombinant DNA techniques well-known in the art. In some embodiments, a nucleic acid molecule is an expression vector that is suitable for expression in a selected host cell.

Also provided herein is a polynucleotide system comprising a nucleic acid sequence encoding each chain of a bispecific antibody, or an antibody or antigen-binding fragment. Also provided herein is a polynucleotide system comprising (a) a first nucleic acid sequence encoding the first heavy chain of a bispecific antibody, and (b) a second nucleic acid sequence encoding the second heavy chain of a bispecific antibody.

In some embodiments, the polynucleotides are comprised in a vector. Vectors comprising polynucleotides that encode binding molecules, including heavy chains and/or light chains of the binding molecules, are provided herein. Vectors comprising polynucleotides that encode binding molecules, including heavy chains and/or light chains of the binding molecules, are also provided herein. In certain embodiments, the vector is selected from the group consisting of a DNA vector, an RNA vector, a plasmid, a lentiviral vector, an adenoviral vector, an adeno-associated viral vector, and a retroviral vector. In certain embodiments, the vector is an expression vector. A nucleic acid of the present disclosure may be present within an expression vector and/or a cloning vector.

An expression vector can include a selectable marker, an origin of replication, and other features that provide for replication and/or maintenance of the vector. Suitable expression vectors include, e.g., plasmids, viral vectors, and the like. Large numbers of suitable vectors and promoters are known to those of skill in the art; many are commercially available for generating a subject recombinant construct. An expression vector may be any suitable vector, including chromosomal, non-chromosomal, and synthetic nucleic acid vectors (a nucleic acid sequence comprising a suitable set of expression control elements). Examples of such vectors include derivatives of SV40, bacterial plasmids, phage DNA, baculovirus, yeast plasmids, vectors derived from combinations of plasmids and phage DNA, and viral nucleic acid (RNA or DNA) vectors. In one embodiment, nucleic acid is comprised in a naked DNA or RNA vector, including, for example, a linear expression element (as described in for instance Sykes and Johnston, Nat Biotech 17, 355-59 (1997)), a compacted nucleic acid vector (as described in for instance U.S. Pat. No. 6,077,835 and/or WO 00/70087), a plasmid vector such as pBR322, pUC 19/18, or pUC 118/119, a “midge” minimally-sized nucleic acid vector (as described in for instance Schakowski et al., Mol Ther 3, 793-800 (2001)), or as a precipitated nucleic acid vector construct, such as a CaPO4-precipitated construct (as described in for instance WO200046147, Benvenisty and Reshef, PNAS USA 83, 9551-55 (1986), Wigler et al., Cell 14, 725 (1978), and Coraro and Pearson, Somatic Cell Genetics 7, 603 (1981)). Such nucleic acid vectors and the usage thereof are well known in the art (see for instance U.S. Pat. Nos. 5,589,466 and 5,973,972).

In one embodiment, the vector is suitable for expression of the polypeptide chains in a bacterial cell. Examples of such vectors include expression vectors such as BlueScript (Stratagene), pIN vectors (Van Heeke & Schuster, J Biol Chem 264, 5503-5509 (1989), pET vectors (Novagen, Madison Wis.) and the like).

An expression vector may also or alternatively be a vector suitable for expression in a yeast system. Any vector suitable for expression in a yeast system may be employed. Suitable vectors include, for example, vectors comprising constitutive or inducible promoters such as alpha factor, alcohol oxidase and PGH (reviewed in: F. Ausubel et al., ed. Current Protocols in Molecular Biology, Greene Publishing and Wiley InterScience New York (1987), and Grant et al., Methods in Enzymol 153, 516-544 (1987)).

An expression vector may also or alternatively be a vector suitable for expression in mammalian cells, e.g. a vector comprising glutamine synthetase as a selectable marker, such as the vectors described in Bebbington (1992) Biotechnology (NY) 10:169-175.

The following vectors are provided by way of example and should not be construed in anyway as limiting: Bacterial: pBs, phagescript, PsiX174, pBluescript SK, pBs KS, pNH8a, pNH16a, pNH18a, pNH46a (Stratagene, La Jolla, Calif., USA); pTrc99A, pKK223-3, pKK233-3, pDR540, and pRIT5 (Pharmacia, Uppsala, Sweden). Eukaryotic: pWLneo, pSV2cat, pOG44, PXR1, pSG (Stratagene) pSVK3, pBPV, pMSG and pSVL (Pharmacia).

Expression vectors generally have convenient restriction sites located near the promoter sequence to provide for the insertion of nucleic acid sequences encoding heterologous proteins. A selectable marker operative in the expression host may be present. Suitable expression vectors include, but are not limited to, viral vectors (e.g. viral vectors based on vaccinia virus; poliovirus; adenovirus (see, e.g., Li et al., Invest. Opthalmol. Vis. Sci. (1994) 35: 2543-2549; Borras et al., Gene Ther. (1999) 6: 515-524; Li and Davidson, Proc. Natl. Acad. Sci. USA (1995) 92: 7700-7704; Sakamoto et al., H. Gene Ther. (1999) 5: 1088-1097; WO 94/12649, WO 93/03769; WO 93/19191; WO 94/28938; WO 95/11984 and WO 95/00655); adeno-associated virus (see, e.g., Ali et al., Hum. Gene Ther. (1998) 9: 81-86, Flannery et al., Proc. Natl. Acad. Sci. USA (1997) 94: 6916-6921; Bennett et al., Invest. Opthalmol. Vis. Sci. (1997) 38: 2857-2863; Jomary et al., Gene Ther. (1997) 4:683 690, Rolling et al., Hum. Gene Ther. (1999) 10: 641-648; Ali et al., Hum. Mol. Genet. (1996) 5: 591-594; Srivastava in WO 93/09239, Samulski et al., J. Vir. (1989) 63: 3822-3828; Mendelson et al., Virol. (1988) 166: 154-165; and Flotte et al., Proc. Natl. Acad. Sci. USA (1993) 90: 10613-10617); SV40; herpes simplex virus; human immunodeficiency virus (see, e.g., Miyoshi et al., Proc. Natl. Acad. Sci. USA (1997) 94: 10319-23; Takahashi et al., J. Virol. (1999) 73: 7812-7816); a retroviral vector (e.g., Murine Leukemia Virus, spleen necrosis virus, and vectors derived from retroviruses such as Rous Sarcoma Virus, Harvey Sarcoma Virus, avian leukosis virus, human immunodeficiency virus, myeloproliferative sarcoma virus, and mammary tumor virus); and the like.

Additional expression vectors suitable for use are, e.g., without limitation, a lentivirus vector, a gamma retrovirus vector, a foamy virus vector, an adeno-associated virus vector, an adenovirus vector, a pox virus vector, a herpes virus vector, an engineered hybrid virus vector, a transposon mediated vector, and the like. Viral vector technology is well known in the art and is described, for example, in Sambrook et al., 2012, Molecular Cloning: A Laboratory Manual, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses.

The vectors can include viral vectors or eukaryotic expression vectors. In some embodiments, the viral vector is a vector derived from simian virus 40 (SV40), adenoviruses, adeno-associated virus (AAV), or retroviral or lentiviral vectors.

In general, a suitable vector contains an origin of replication functional in at least one organism, a promoter sequence, convenient restriction endonuclease sites, and one or more selectable markers, (e.g., WO 01/96584; WO 01/29058; and U.S. Pat. No. 6,326,193).

A nucleic acid and/or vector may also comprises a nucleic acid sequence encoding a secretion/localization sequence, which can target a polypeptide, such as a nascent polypeptide chain, to the periplasmic space or into cell culture media. Such sequences are known in the art, and include secretion leader or signal peptides.

The expression vector may comprise or be associated with any suitable promoter, enhancer, and other expression-facilitating elements. Examples of such elements include strong expression promoters (e. g., human CMV IE promoter/enhancer as well as RSV, SV40, SL3-3, MMTV, and HIV LTR promoters), effective poly (A) termination sequences, an origin of replication for plasmid product in E. coli, an antibiotic resistance gene as selectable marker, and/or a convenient cloning site (e.g., a polylinker). Nucleic acids may also comprise an inducible promoter as opposed to a constitutive promoter such as CMV IE.

In a further embodiment, a host cell comprising such polynucleotides is provided. In one such embodiment, a host cell comprises (e.g., has been transformed with) a vector or vectors that comprise one or more polynucleotides that encodes an amino acid sequence comprising the binding molecule or a chain thereof. In some embodiments, a host cell comprises (e.g., has been transformed with) one or more vectors comprising one or more polynucleotide that encodes one or more an amino acid sequence comprising one or more antibodies and/or portions thereof, e.g., antigen-binding fragments thereof. In some embodiments, a host cell comprises (e.g., has been transformed with) (1) a vector comprising a polynucleotide that encodes an amino acid sequence comprising the VL region of the antibody and an amino acid sequence comprising the VH region of the antibody, or (2) a first vector comprising a polynucleotide that encodes an amino acid sequence comprising the VL region of the antibody and a second vector comprising a polynucleotide that encodes an amino acid sequence comprising the VH region of the antibody. In some embodiments, one or more such host cells are provided. In some embodiments, a composition containing one or more such host cells are provided.

In some embodiments, the host cell may comprise the nucleic acid molecules encoding the polypeptides stably integrated into the cellular genome. In another embodiment, the cell is transformed to contain non-integrated nucleic acid, such as a plasmid, cosmid, phagemid, or linear expression element.

In some embodiments, a vector comprises a first polynucleotide sequence encoding a heavy chain and a second polynucleotide sequence encoding a light chain. In some embodiments, the heavy chain and light chain are expressed from the vector as two separate polypeptides. In some embodiments, the heavy chain variable domain fragment and light chain variable domain fragment are expressed as part of a single polypeptide, such as, for example, when the antibody is an scFv.

In some embodiments, a first vector comprises a polynucleotide that encodes a heavy chain and a second vector comprises a polynucleotide that encodes a light chain. In some embodiments, the first vector and second vector are transfected into host cells in similar amounts (such as similar molar amounts or similar mass amounts). In some embodiments, a mole- or mass-ratio of between 5:1 and 1:5 of the first vector and the second vector is transfected into host cells. In some embodiments, a mass ratio of between 1:1 and 1:5 for the vector encoding the heavy chain and the vector encoding the light chain is used. In some embodiments, a mass ratio of 1:2 for the vector encoding the heavy chain and the vector encoding the light chain is used.

Also provided herein is a vector system comprising a plurality of vectors, each vector comprising a nucleic acid sequence of the polynucleotide system.

Also provided is a host cell comprising any of the vectors or nucleic acids disclosed herein. The host cell may be of eukaryotic, prokaryotic, mammalian, or bacterial origin. In various embodiments, heavy chains and/or light chains may be expressed in prokaryotic cells, such as bacterial cells; or in eukaryotic cells, such as fungal cells (such as yeast), plant cells, insect cells, and mammalian cells. In some embodiments, the cell is a mammalian cell. Such expression may be carried out, for example, according to procedures known in the art. Exemplary eukaryotic cells that may be used to express polypeptides include, but are not limited to, COS cells, including COS 7 cells; HEK293 cells, including HEK293-6E cells; CHO cells, including CHO-S, DG44. Lecl3 CHO cells, and FUT8 CHO cells; PER.C6® cells (Crucell); and NS0 cells. In some embodiments, heavy chains and/or light chains may be expressed in yeast. See, e.g., U.S. Publication No. US 2006/0270045 A1. In some embodiments, a particular eukaryotic host cell is selected based on its ability to make desired post-translational modifications to the encoded polypeptide, including to heavy chains and/or light chain domains. For example, in some embodiments, CHO cells produce polypeptides that have a higher level of sialylation than the same polypeptide produced in HEK293 cells.

In some embodiments, the antibody or antigen-binding fragment provided herein is produced in a cell-free system. Exemplary cell-free systems are described, e.g., in Sitaraman et al., Methods Mol. Biol. 498: 229-44 (2009); Spirin, Trends Biotechnol. 22: 538-45 (2004); Endo et al., Biotechnol. Adv. 21: 695-713 (2003).

Introduction of one or more nucleic acids into a desired host cell may be accomplished by any method, including but not limited to, calcium phosphate transfection, DEAE-dextran mediated transfection, cationic lipid-mediated transfection, electroporation, transduction, infection, etc. Nonlimiting exemplary methods are described, e.g., in Sambrook et al., Molecular Cloning, A Laboratory Manual, 3^{rd} ed. Cold Spring Harbor Laboratory Press (2001). Nucleic acids may be transiently or stably transfected in the desired host cells, according to any suitable method.

Also provided herein are host cells comprising any of the polynucleotides or vectors described herein. In some embodiments host cells are capable of over-expressing heterologous DNAs can be used for the purpose of isolating the genes encoding the antibody, polypeptide or protein of interest. Non-limiting examples of mammalian host cells include but not limited to COS, HeLa, and CHO cells. See also PCT Publication No. WO 87/04462. Suitable non-mammalian host cells include prokaryotes (such as E. coli or B. subtilis) and yeast (such as S. cerevisiae, S. pombe; or K. lactis).

A method of producing a binding molecule, such as a CD19 monospecific antibody binding domain or a bispecific binding molecule having an anti-CD19 antibody binding domain specific to CD19 and an anti-CD3 antibody binding domain specific to CD3, including an antibody or antigen-binding fragment of any such molecules, is also provided herein, wherein the method comprises culturing any of the provided host cells. In some embodiments, the nucleic acids encoding a subject binding molecule, including an antibody or antigen-binding fragment, are introduced directly into a host cell, and the cell incubated under conditions sufficient to induce expression of the encoded antibody. Expression may conveniently be achieved by culturing under appropriate conditions recombinant host cells containing the nucleic acid. Following production by expression, the binding molecule, including an antibody or antigen-binding fragment thereof, may be isolated and/or purified using any suitable technique, and then used as desired.

The provided binding molecules, including bispecific antibodies, may be purified by any suitable method. Such methods include, but are not limited to, the use of affinity matrices or hydrophobic interaction chromatography. Suitable affinity ligands such as ligands that bind antibody constant regions. For example, a Protein A, Protein G, Protein A/G, or an antibody affinity column may be used to bind the constant region and to purify a CD3 binding domain. Hydrophobic interactive chromatography, for example, a butyl or phenyl column, may also be suitable for purifying some polypeptides such as antibodies. Ion exchange chromatography (e.g., anion exchange chromatography and/or cation exchange chromatography) may also be suitable for purifying some polypeptides such as antibodies.

Mixed-mode chromatography (e.g., reversed phase/anion exchange, reversed phase/cation exchange, hydrophilic interaction/anion exchange, hydrophilic interaction/cation exchange, etc.) may also be suitable for purifying some polypeptides such as antibodies. Many methods of purifying polypeptides are known in the art. In some embodiments, the method further comprises recovering the antibody produced by the cell.

Antibodies or antigen-binding fragments thereof as provided herein, and encoding nucleic acid molecules and vectors, may be isolated and/or purified, e.g. from their natural environment, in substantially pure or homogeneous form, or, in the case of nucleic acid, free or substantially free of nucleic acid or genes of origin other than the sequence encoding a polypeptide with the desired function.

In some embodiments, the bispecific antibodies provided herein can be obtained by co-expression of constructs encoding the polypeptides of the bispecific antibody in a single host cell. In some embodiments, the methods include co-expressing the first polypeptide comprising the first heavy chain, the second polypeptide comprising the second heavy chain, and the polypeptide(s) comprising the light chain. In some embodiments, nucleic acids encoding the polypeptide chains are incorporated into expression vectors for introducing into the host cell as described. By virtue of the Fc region of the first heavy chain and second heavy chain being a different polypeptide chain of a heterodimeric Fc each containing different mutations in their CH3 region to promote heterodimeric interactions, the polypeptide chains of the first and second heavy chain will associate to form the bispecific antibody. Moreover, the light chain also will associate with the respective heavy chains. The resulting bispecific antibodies can be purified by any suitable method such as, for example, by affinity chromatography over Protein A or Protein G columns. Where two nucleic acid molecules encoding different polypeptides are transformed into cells, formation of homo- and heterodimers will occur. Conditions for expression can be adjusted so that heterodimer formation is favored over homodimer formation.

Techniques for recovery of heterodimers from homodimers based on a differential affinity of the heterodimers for an affinity reagent are known. In some aspects, such techniques include designing a heterodimer so that one of the Fc polypeptide chains does not bind to the affinity reagent protein A. In some cases, one of the polypeptide chain can contain one or more amino acid substitution to abrogate or reduce affinity for the protein A reagent in one of the polypeptides of the Fc heterodimer, see e.g. WO2017134440, WO2010151792, Jendeberg et al. (Jendeberg et al., (1997) J. Immunol. Methods, 201(1): 25-34. In some of these embodiments, the Fc region may be modified at the protein-A binding site on one member of the heterodimer so as to prevent protein-A binding and thereby enable more efficient purification of the heterodimeric fusion protein.

In some embodiments, separate plasmids encoding each chain of the bispecific antibody are transfected at an equimolar ratio into mammalian cells (such as HEK293 or CHO). Recombinant protein is secreted into the supernatant and can be collected, such as after 3-7 days. In some embodiments, the antibody is purified by protein A chromatography, followed by either preparative size exclusion chromatography (SEC) or flow-through hydrophobic interaction chromatography (HIC). In some embodiments, a second chromatography step may be carried out, such as on SEC (AKTA with Superdex-200 resin) or FT-HIC (AKTA with butyl/phenyl sepharose), to remove undesired cross-paired species.

In some embodiments, the bispecific antibody is a purified bispecific antibody.

### III. PHARMACEUTICAL COMPOSITIONS

Provided herein are compositions containing any of the provided antibodies or antigen-binding fragments or binding molecules containing same as described herein. The pharmaceutical composition can further comprise a pharmaceutically acceptable excipient. For example, the pharmaceutical composition can contain one or more excipients for modifying, maintaining or preserving, for example, the pH, osmolarity, viscosity, clarity, color, isotonicity, odor, sterility, stability, rate of dissolution or release, adsorption, or penetration of the composition. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (e.g., aluminum hydroxide); and preservatives.

In some embodiments, the pharmaceutical composition is a solid, such as a powder, capsule, or tablet. For example, the components of the pharmaceutical composition can be lyophilized. In some embodiments, the solid pharmaceutical composition is reconstituted or dissolved in a liquid prior to administration.

In some embodiments, the pharmaceutical composition is a liquid, for example a provided protein dissolved in an aqueous solution (such as physiological saline or Ringer's solution). In some embodiments, the pH of the pharmaceutical composition is between about 4.0 and about 8.5 (such as between about 4.0 and about 5.0, between about 4.5 and about 5.5, between about 5.0 and about 6.0, between about 5.5 and about 6.5, between about 6.0 and about 7.0, between about 6.5 and about 7.5, between about 7.0 and about 8.0, or between about 7.5 and about 8.5).

In some embodiments, the pharmaceutical composition comprises a pharmaceutically acceptable excipient, for example a filler, binder, coating, preservative, lubricant, flavoring agent, sweetening agent, coloring agent, a solvent, a buffering agent, a chelating agent, or stabilizer. Examples of pharmaceutically acceptable fillers include cellulose, dibasic calcium phosphate, calcium carbonate, microcrystalline cellulose, sucrose, lactose, glucose, mannitol, sorbitol, maltol, pregelatinized starch, corn starch, or potato starch. Examples of pharmaceutically acceptable binders include polyvinylpyrrolidone, starch, lactose, xylitol, sorbitol, maltitol, gelatin, sucrose, polyethylene glycol, methyl cellulose, or cellulose. Examples of pharmaceutically acceptable coatings include hydroxypropyl methylcellulose (HPMC), shellac, corn protein zein, or gelatin. Examples of pharmaceutically acceptable disintegrants include polyvinylpyrrolidone, carboxymethyl cellulose, or sodium starch glycolate. Examples of pharmaceutically acceptable lubricants include polyethylene glycol, magnesium stearate, or stearic acid. Examples of pharmaceutically acceptable preservatives include methyl parabens, ethyl parabens, propyl paraben, benzoic acid, or sorbic acid. Examples of pharmaceutically acceptable sweetening agents include sucrose, saccharine, aspartame, or sorbitol. Examples of pharmaceutically acceptable buffering agents include carbonates, citrates, gluconates, acetates, phosphates, or tartrates.

In some embodiments, the pharmaceutical composition further comprises an agent for the controlled or sustained release of the product, such as injectable microspheres, bio-erodible particles, polymeric compounds (polylactic acid, polyglycolic acid), beads, or liposomes.

In some embodiments, the pharmaceutical composition is sterile. Sterilization may be accomplished by filtration through sterile filtration membranes or radiation. Where the composition is lyophilized, sterilization using this method may be conducted either prior to or following lyophilization and reconstitution. The composition for parenteral administration may be stored in lyophilized form or in solution. In addition, parenteral compositions generally are placed into a container having a sterile access port, for example, an intravenous solution bag or vial having a stopper pierceable by a hypodermic injection needle.

A pharmaceutically acceptable carrier may be a pharmaceutically acceptable material, composition, or vehicle. For example, the carrier may be a liquid or solid filler, diluent, excipient, solvent, or encapsulating material, or some combination thereof. Each component of the carrier must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits.

In some embodiments, the pharmaceutical composition is for administration to a subject. Generally, dosages and routes of administration of the pharmaceutical composition are determined according to the size and condition of the subject, according to standard pharmaceutical practice. For example, the therapeutically effective dose can be estimated initially either in cell culture assays or in animal models such as mice, rats, rabbits, dogs, pigs, or monkeys. An animal model may also be used to determine the appropriate concentration range and route of administration. Such information can then be used to determine useful doses and routes for administration in humans. The exact dosage will be determined in light of factors related to the subject requiring treatment. Dosage and administration are adjusted to provide sufficient levels of the active compound or to maintain the desired effect. Factors that may be taken into account include the severity of the disease state, the general health of the subject, the age, weight, and gender of the subject, time and frequency of administration, drug combination(s), reaction sensitivities, and response to therapy.

Long-acting pharmaceutical compositions may be administered every 3 to 4 days, every week, or biweekly depending on the half-life and clearance rate of the particular formulation. The frequency of dosing will depend upon the pharmacokinetic parameters of the molecule in the formulation used. Typically, a composition is administered until a dosage is reached that achieves the desired effect. The composition may therefore be administered as a single dose, or as multiple doses (at the same or different concentrations/dosages) over time, or as a continuous infusion. Further refinement of the appropriate dosage is routinely made. Appropriate dosages may be ascertained through use of appropriate dose-response data.

In some embodiments, the pharmaceutical composition is for administration to a subject through any route, including orally, transdermally, by inhalation, intravenously, intra-arterially, intramuscularly, direct application to a wound site, application to a surgical site, intraperitoneally, by suppository, subcutaneously, intradermally, transcutaneously, by nebulization, intrapleurally, intraventricularly, intra-articularly, intraocularly, or intraspinally.

A provided pharmaceutical formulation may, for example, be in a form suitable for intravenous infusion.

In some embodiments, the dosage of the pharmaceutical composition is a single dose or a repeated dose. In some embodiments, the doses are given to a subject once per day, twice per day, three times per day, or four or more times per day. In some embodiments, about 1 or more (such as about 2 or more, about 3 or more, about 4 or more, about 5 or more, about 6 or more, or about 7 or more) doses are given in a week. In some embodiments, multiple doses are given over the course of days, weeks, months, or years. In some embodiments, a course of treatment is about 1 or more doses (such as about 2 or more does, about 3 or more doses, about 4 or more doses, about 5 or more doses, about 7 or more doses, about 10 or more doses, about 15 or more doses, about 25 or more doses, about 40 or more doses, about 50 or more doses, or about 100 or more doses).

In some embodiments, an administered dose of the pharmaceutical composition is about 1 μg of protein per kg subject body mass or more (such as about 2 μg of protein per kg subject body mass or more, about 5 μg of protein per kg subject body mass or more, about 10 μg of protein per kg subject body mass or more, about 25 μg of protein per kg subject body mass or more, about 50 μg of protein per kg subject body mass or more, about 100 μg of protein per kg subject body mass or more, about 250 μg of protein per kg subject body mass or more, about 500 μg of protein per kg subject body mass or more, about 1 mg of protein per kg subject body mass or more, about 2 mg of protein per kg subject body mass or more, or about 5 mg of protein per kg subject body mass or more).

### IV. METHODS OF USE

The CD3 and CD19-binding domains and antigen-binding fragments or binding molecules containing the same described herein, including pharmaceutical compositions thereof, can be used in a variety of therapeutic applications, such as the treatment of a disease. Such methods and uses include therapeutic methods and uses, for example, involving administration of the molecules or compositions containing the same, to a subject having a disease or disorder. In some embodiments, the binding molecule is administered in an effective amount to effect treatment of the disease or disorder. Uses include uses of the CD3 and CD19-binding domains and antigen-binding fragments or binding molecules containing the same described herein, including pharmaceutical compositions thereof, in such methods and treatments, and in the preparation of a medicament in order to carry out such therapeutic methods. In some embodiments, the methods are carried out by administering the CD3 and CD19-binding domains and antigen-binding fragments or binding molecules containing the same, including pharmaceutical compositions thereof, or compositions comprising the same, to the subject having or suspected of having the disease or disorder. In some embodiments, the methods thereby treat the disease or disorder in the subject. Also provided are any methods or uses involving administering a provided binding molecule as described, or pharmaceutical compositions containing the same, for treating a disease or disorder.

Illustrative subjects include mammalian subjects, such as farm animals, domestic animals, and human patients. In particular embodiments, the subject is a human subject. The terms subject and patient are used interchangeably herein.

In some embodiments, the disclosure provides methods of treating a subject in need thereof by administering a bispecific antibody that specifically binds CD3 and CD19. In certain embodiments, the bispecific antibody is capable of binding CD19 on a target cell (e.g., a CD19-positive tumor cell or other CD19-positive B-lineage cell) and CD3 on a T cell, thereby physically bridging the T cell and the target cell. Without being bound by theory, such bridging promotes formation of an immune synapse, activation of the T cell, and redirected cytotoxic activity toward the CD19-positive target cell, resulting in depletion of B cells and/or plasmablasts. In some embodiments, the bispecific antibody induces one or more of: T-cell activation, T-cell proliferation, cytokine release, and/or target-cell killing in the presence of CD19-positive cells.

In some cases, a subject is selected that is known, suspected or that has been identified as having a tumor. In some cases, a subject is selected that is known, suspected or that has been identified as having an autoimmune disease or inflammatory condition. A provided CD3 and CD19-binding domain or antigen-binding fragment or binding molecule as described herein, or pharmaceutical composition comprising the same, is administered to the subject. The administration to the subject will generally have an effect due to its binding with the CD3 target.

Generally, alleviation or treatment of a disease or disorder involves the lessening of one or more symptoms or medical problems associated with the disease or disorder. For example, in the case of cancer, the therapeutically effective amount of the drug can accomplish one or a combination of the following: reduce the number of cancer cells; reduce the tumor size; inhibit (i.e., to decrease to some extent and/or stop) cancer cell infiltration into peripheral organs; inhibit tumor metastasis; inhibit, to some extent, tumor growth; and/or relieve to some extent one or more of the symptoms associated with the cancer. In some embodiments, a composition of this disclosure can be used to prevent the onset or reoccurrence of the disease or disorder in the subject.

In some embodiments, the CD3 and CD19-binding domains or antigen-binding fragment or binding molecule as described herein, or pharmaceutical composition comprising the same, can be used to inhibit growth of mammalian cancer cells (such as human cancer cells). A method of treating cancer can include administering an effective amount of any of the pharmaceutical compositions described herein to a subject with cancer. The effective amount of the pharmaceutical composition can be administered to inhibit, halt, or reverse progression of cancers. Human cancer cells can be treated in vivo, or ex vivo. In ex vivo treatment of a human patient, tissue or fluids containing cancer cells are treated outside the body and then the tissue or fluids are reintroduced back into the patient. In some embodiments, the cancer is treated in a human patient in vivo by administration of the therapeutic composition into the patient.

In some embodiments, the method is used to treat a cancer comprising CD19-positive malignant cells. Exemplary cancers include B-cell malignancies such as B-cell acute lymphoblastic leukemia (B-ALL), chronic lymphocytic leukemia (CLL), small lymphocytic lymphoma (SLL), diffuse large B-cell lymphoma (DLBCL), follicular lymphoma, mantle cell lymphoma, marginal zone lymphoma, Waldenstrom macroglobulinemia/lymphoplasmacytic lymphoma, and other CD19-positive non-Hodgkin lymphomas.

In some embodiments, the CD3 and CD19-binding domains or antigen-binding fragment or binding molecule as described herein, or pharmaceutical composition comprising the same, are useful in treating, alleviating a symptom of, ameliorating and/or delaying the progression of a disease or disorder. In some embodiments, the disease or disorder is a cancer, tumor or other neoplastic condition. In some embodiments, the cancer is bladder cancer, breast cancer, gastric cancer, uterine/cervical cancer, ovarian cancer, prostate cancer, testicular cancer, esophageal cancer, gastrointestinal cancer, pancreatic cancer, colorectal cancer, colon cancer, kidney cancer, head and neck cancer, lung cancer, stomach cancer, germ cell cancer, bone cancer, liver cancer, thyroid cancer, skin cancer, neoplasm of the central nervous system, lymphoma, leukemia, myeloma, sarcoma, and virus-related cancer. In some embodiments, the cancer is a melanoma, breast carcinoma, HCC, Renal cell carcinoma, Colorectal carcinoma, non-small-cell lung carcinoma, Cervical cancer or Mesothelioma. In some embodiments, the cancer is ovarian cancer. In certain embodiments, the cancer is a metastatic cancer, refractory cancer, or recurrent cancer. In some embodiments, the cancer is a solid tumor.

In some embodiments, the CD3 and CD19-binding domains or antigen-binding fragment or binding molecule as described herein, or pharmaceutical composition comprising the same, are useful in treating, alleviating a symptom of, ameliorating and/or delaying the progression of a disease or disorder. In some embodiments, the disease or disorder is an autoimmune disease or inflammatory disease. In some embodiments, the disease or condition is associated with overexpression of B cells in tissues. In some embodiments, the autoimmune disorder is associated with presence of increased autoantibodies and/or autoreactive B cells in the subject compared to a healthy subject. In some embodiments, the method is used to treat an autoimmune disease or inflammatory condition associated with pathological B-cell activity, including autoantibody production, aberrant antigen presentation, and/or pro-inflammatory cytokine signaling. In certain embodiments, the subject has elevated circulating plasmablasts, increased autoantibody titers, or evidence of B-cell-driven pathology, and administration of the bispecific antibody reduces one or more clinical signs or laboratory markers of disease activity. In some embodiments, the method reduces autoantibody titers and/or decreases the frequency of disease flares relative to baseline or to standard-of-care therapy.

In some embodiments, results described herein support the treatment of autoimmune and inflammatory diseases and disorders using a provided bispecific antibody described herein to deplete CD19+ B cells while exhibiting a desired safety signal (e.g., reduced induction of pro-inflammatory cytokines). As described in Example 19, representative bispecific antibodies can mediate robust B cell depletion in human immune cell-based assays while inducing relatively low cytokine release at concentrations that achieve therapeutically relevant depletion, and further exhibit improved separation between B cell depletion and cytokine induction as compared to comparator molecules evaluated in the same assay. Such a profile may be particularly advantageous for autoimmune and inflammatory indications, where patients may require repeated and/or chronic dosing and where excessive systemic cytokine induction can limit tolerability, constrain dose selection, and increase the risk of treatment-emergent inflammatory toxicities or disease exacerbation. Accordingly, in some embodiments, provided bispecific antibodies described herein are suitable for treatment of B cell-mediated autoimmune disorders by enabling therapeutically effective B cell depletion with a reduced risk of cytokine-associated adverse events and improved dosing flexibility.

In some embodiments, the autoimmune disease or inflammatory disease includes, but is not limited to, psoriasis, psoriatic arthritis, Type 1 diabetes mellitus (Type 1 diabetes), rheumatoid arthritis (RA), human systemic lupus (SLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus, dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

In some embodiments, the systemic autoimmune diseases may include Systemic Lupus Erythematosus (SLE), Sjögren's′ syndrome, progressive systemic sclerosis (i.e., scleroderma), IIM, including dermatomyositis, polymyositis and immune-mediated necrotizing myositis, MCTD, relapsing-remitting multiple sclerosis, AAV, Crohn's disease, myasthenia gravis, Behçet's disease, rheumatoid arthritis, primary progressive MS, IgA nephropathy, pemphigus vulgaris, myasthenia gravis, autoimmune hemolytic anemia, immune thrombocytopenia, IgG4-related diseases, membranous nephropathy, cutaneous lupus erythematosus, sarcoidosis, light chain amyloidosis, acute respiratory distress syndrome, atopic eczema, hereditary angioedema, hidradenitis suppurative, inclusion-body myositis, inflammatory bowel disease, mastocytosis, multifocal motor neuropathy, necrotizing myopathy, neuromyelitis optica spectrum disorder, mixed connective tissue disorder, POEMS syndrome, primary biliary cholangitis, psoriasis, rhesus hemolytic disease, Still's disease, type 1 diabetes, urticaria, capillary leakage syndrome, erythema multiforme, pyoderma gangrenosum, x-linked agammaglobulinemia, antiphospholipid syndrome, and chronic inflammatory demyelinating polyneuropathy (also called chronic inflammatory demyelinating polyradiculoneuropathy). Exemplary autoimmune diseases include systemic lupus erythematosus, lupus nephritis, rheumatoid arthritis, Sjögren's syndrome, systemic sclerosis, idiopathic inflammatory myopathies, vasculitides, autoimmune hemolytic anemia, immune thrombocytopenia, autoimmune thyroid disease, multiple sclerosis, and/or myasthenia gravis. In some embodiments, the autoimmune disorder is systemic lupus erythematosus (SLE), rheumatoid arthritis, Sjogren disease, or systemic sclerosis.

In some embodiments, the autoimmune disease is SLE, such as a moderate SLE or severe refractory SLE, idiopathic inflammatory myopathy, systemic sclerosis, rheumatoid arthritis (RA) or multiple sclerosis. In some embodiments, the systemic autoimmune disease is SLE, such as a moderate SLE or severe refractory SLE, idiopathic inflammatory myopathy, systemic sclerosis, or multiple sclerosis. Among provided methods are methods of treatment that involve administering the binding molecules (e.g., bispecific binding molecules) to subjects with SLE, including severe refractory SLE. Also provided are methods and uses of provided binding molecules (e.g., bispecific binding molecules) and/or compositions thereof, including methods for the treatment of subjects having a SLE, including severe refractory SLE, that involves administration of the binding molecules and/or compositions thereof. In certain embodiments, the subject has severe refractory SLE. In some embodiments, the subject is selected for or identified as having severe refractory SLE, such as by the presence of certain features or clinical manifestations that indicate the presence of severe refractory SLE. Exemplary selection criteria are further described herein. In some embodiments, the methods and use of provided binding molecules and/or compositions thereof, include methods for the treatment of subjects with severe refractory SLE. In particular embodiments, the method includes administering to the subject a dose of binding molecules, wherein the binding molecules contain a domain that specifically binds to CD19 and CD3.

In some embodiments, efficacy of the bispecific antibody is characterized by depletion of B cells and/or plasmablasts in one or more anatomical compartments, including lymph node, bone marrow, and/or peripheral blood. In some embodiments, the bispecific antibody results in B cell and/or plasmablast depletion of greater than 90% in the lymph node, bone marrow, and/or blood, as determined by an in vitro cell depletion assay. In certain embodiments, the in vitro cell depletion assay comprises co-culturing CD19-positive target cells with T cells in the presence of the bispecific antibody and quantifying residual target cells after a defined incubation period (e.g., about 4 hours, about 24 hours, about 48 hours, or longer). In some embodiments, depletion is quantified by flow cytometry using one or more B-cell markers (e.g., CD19, CD20, CD22, CD79a/b) and/or plasmablast markers (e.g., CD19^{low/-}, CD20⁻, CD27^{high}, CD38^{high}) and reported as percent depletion relative to a control condition lacking the bispecific antibody or lacking effector T cells.

Compositions of the invention can be administered in dosages and routes and at times to be determined in appropriate pre-clinical and clinical experimentation and trials. Compositions may be administered multiple times at dosages within these ranges. Administration of the compositions may be combined with other methods useful to treat the desired disease or condition as determined by those of skill in the art. Typically, precise amount of the compositions of the present disclosure to be administered can be determined by a physician with consideration of individual differences in age, weight, tumor size, extent of infection or metastasis, and condition of the patient (subject).

In some embodiments, a therapeutically effective dose may be, by way of nonlimiting example, using body weight.

In some embodiments, a therapeutic amount of a binding molecule composition of the present disclosure is administered. Binding molecule compositions may also be administered multiple times at these dosages. Any suitable method or route can be used to administer a presently disclosed binding molecules. Routes of administration include, for example, oral, intravenous, intraperitoneal, subcutaneous, or intramuscular administration. It should be emphasized, however, that the presently disclosed subject matter is not limited to any particular method or route of administration. The optimal dosage and treatment regime for a particular patient can readily be determined by one skilled in the art of medicine by monitoring the patient for signs of disease and adjusting the treatment accordingly.

The CD3 and CD19-binding domains or antigen-binding fragment or binding molecule, or pharmaceutical composition comprising the same, of the present disclosure can be administered alone or in combination with other modes of treatment, such as other anti-cancer agents. They can be provided before, substantially contemporaneous with, or after other modes of treatment (i.e., concurrently or sequentially). In some embodiments, the method of treatment described herein can further include administering: radiation therapy, chemotherapy, vaccination, targeted tumor therapy, CAR-T therapy, oncolytic virus therapy, cancer immunotherapy, cytokine therapy, surgical resection, chromatin modification, ablation, cryotherapy, an antisense agent against a tumor target, a siRNA agent against a tumor target, a microRNA agent against a tumor target or an anti-cancer/tumor agent, or a biologic, such as an antibody, cytokine, or receptor extracellular domain-Fc fusion.

### V. ARTICLES OF MANUFACTURE OR KITS

Provided herein are articles of manufacture and kits comprising the provided compositions In some embodiments, the kit comprises any of the provided compositions and instructions for administering the composition to a subject.

Kits can optionally include one or more components such as instructions for use, devices and additional reagents (e.g., sterilized water or saline solutions for dilution of the compositions and/or reconstitution of lyophilized protein), and components, such as tubes, containers and syringes for practice of the methods. In some embodiments, the kits can further contain reagents for collection of samples, preparation and processing of samples, and/or reagents for quantitating the amount of one or more surface markers in a sample, such as, but not limited to, detection reagents, such as antibodies, buffers, substrates for enzymatic staining, chromagens or other materials, such as slides, containers, microtiter plates, and optionally, instructions for performing the methods. Those of skill in the art will recognize many other possible containers and plates and reagents that can be used in accord with the provided methods.

In some embodiments, the kits can be provided as articles of manufacture that include packing materials for the packaging of the cells, antibodies or reagents, or compositions thereof, or one or more other components. For example, the kits can contain containers, bottles, tubes, vial and any packaging material suitable for separating or organizing the components of the kit. The one or more containers may be formed from a variety of materials such as glass or plastic. In some embodiments, the one or more containers hold a composition comprising cells or an antibody or other reagents for use in the methods. The article of manufacture or kit herein may comprise the cells, antibodies or reagents in separate containers or in the same container.

In some embodiments, the one or more containers holding the composition may be a single-use vial or a multi-use vial, which, in some cases, may allow for repeat use of the composition. In some embodiments, the article of manufacture or kit may further comprise a second container comprising a suitable diluent. The article of manufacture or kit may further include other materials desirable from a commercial, therapeutic, and user standpoint, including other buffers, diluents, filters, needles, syringes, therapeutic agents and/or package inserts with instructions for use.

In some embodiments, the kit can, optionally, include instructions. Instructions typically include a tangible expression describing the cell composition, reagents and/or antibodies and, optionally, other components included in the kit, and methods for using such. In some embodiments, the instructions indicate methods for using the cell compositions and antibodies for administration to a subject for treating a disease or condition, such as in accord with any of the provided embodiments. In some embodiments, the instructions are provided as a label or a package insert, which is on or associated with the container. In some embodiments, the instructions may indicate directions for reconstitution and/or use of the composition.

### VL EXAMPLES

### Example 1: Identification of Anti-CD19 Antibodies

Human anti-CD19 monoclonal antibodies (mAbs) were generated by immunizing human BCR transgenic mouse strains. Cohorts of mice were immunized with one or more of the following immunogens, e.g., DNA encoding SEQ ID NO: 1 (human CD19), DNA encoding SEQ ID NO: 3 (Macaca fascicularis CD19), a human CD19-human serum albumin fusion protein (CD19-Alb2-12×His; SEQ ID NO: 29), Raji cells (expressing human CD19), and/or a cyno CD19/CD81 CHOK1 overexpression cell line. Spleen and/or lymph node cells from the immunized mice were harvested and antibodies specific to CD19 were identified by hybridoma or single B cell cloning technologies as described below.

In order to discover anti-CD19 antibodies via the hybridoma method, three human BCR transgenic mice were immunized with DNA encoding full-length human CD19, followed by CD19-Alb2-12×His and Raji cells. In addition, four human BCR transgenic mice were immunized with human CD19 and cyno CD19 DNA, followed by CD19-Alb2-12×His, Raji cells, and cyno CD19/CD81 CHOK1 overexpressing cells. Hybridomas were generated by fusing lymphocytes harvested from mice with a P3X63Ag8U.1-derived myeloma cell line using electrofusion. Fused cells were plated into multi-well plates in selective hypoxanthine-aminopterin-thymidine (HAT) medium for seven days. The hybridoma supernatants were subsequently screened by ELISA for binding to CD19-Alb2-12×His (detection by peroxidase F(ab′)₂ fragment goat anti-mouse IgG, Fcγ fragment specific) and/or binding to human CD19/CD81 CHOK1 and cyno CD19/CD81 CHOK1 cells using flow cytometry. A total of 333 ELISA and/or flow cytometry positive hybridoma wells were sequenced via barcoded Next Generation Sequencing (NGS, MiSeq). Based on the ELISA (data not shown), flow cytometry (data not shown), and sequence results (data not shown), 16 antibody sequences were selected to move forward for recombinant expression.

In addition to hybridoma, a single B cell cloning (SBC) approach was utilized to isolate CD19-specific mAbs from the mice immunized as described above. The lymphocytes and/or splenocytes from the immunized mice were incubated, stained with markers identifying live/dead cells, markers identifying IgG-positive class-switched memory B-cells, as well as CD19-Alb2-12×His antigen (SEQ ID NO: 29), which was designed as disclosed in Lobner et al, Frontiers in Bioengineering and Biotechnology 8: 49 (2020). This molecule contains a native CD19 signal peptide followed by the coding sequence of the extracellular domain of human CD19 (UniProt ID: P15391, residues P20-K291), a glycine-serine linker (G4S)3, the domain 2 of HSA (UniProt ID: P02768, residues D211-P413), a GTS linker and a 12× histidine tag. Using a FACS sorter, antigen-specific B cells were individually sorted at 1 cell/well into multi-well plates containing lysis buffer, thereby lysing the cells, and releasing the RNA. Lysates were subjected to reverse transcription and two rounds of PCR to isolate the VH and VL regions of the captured BCR (B cell receptor) for NGS sequencing. The third and final PCR reaction appended promoter/signal peptide and constant region blocks to the requisite ends of the variable region to generate Transcriptionally Active PCR (TAP) (Liang et al., J Biol Chem. 2002: 277 (5): 3593-8) products. The TAP products were then transfected via high-throughput methods into Expi293 cells and purified using Protein A to generate small-scale amounts of recombinant hIgG1 antibody material for screening by flow cytometry, with a total of fourteen 96-well plates generated.

The V_{H} and V_{L} regions from the sorted B cells were also recovered in parallel and sequenced by Next Generation Sequencing (NGS) using a MiSeq sequencing system (Illumina). The recombinant IgG proteins were tested for binding to human CD19/CD81 CHOK1 and cyno CD19/CD81 CHOK1 cells using flow cytometry (data not shown). Based on the flow cytometry (data not shown) and sequence results (data not shown), thirteen (13) antibody sequences were selected to move forward for plasmid-based recombinant expression.

### Example 2: Natural Affinity Maturation of Anti-CD19 Antibodies

This Example describes methods of grouping antibodies by clonotype families in which members in one family have the same variable heavy V and J genes, the same variable light V and J genes, and the same HCDR3 amino acid sequence length. High-affinity variants of mAbs can be rapidly obtained by testing of somatic sequence variants obtained by deep sequencing of antibody variable regions in immune repertories from immunized mice, even with a relatively sparse sampling of sequence variants from large sequence datasets. Methods similar to those used in this Example were described in Hsiao et al. (Immune repertoire mining for rapid affinity optimization of mouse monoclonal antibodies. MABS, 2019, Vol. 11, No. 4, 735-746).

Based on analysis of antibody sequences from Example 1, three clonotype groups of interest were identified based on sequence prevalence, and multiple antibody sequences per clonotype group were chosen as reference antibodies for the natural affinity maturation method (see Table E0).

TABLE E0
CD19 Reference Groups by Clonotype Properties

| | VH | VH | HCDR3 | VL | VL | Ref Abs |
|---|---|---|---|---|---|---|
| | V-gene | J-gene | length | V-gene | J-gene | chosen |
| Group 1 | IGHV2-5*09 | IGHJ6*02 | 16 | IGKV1-39*01 | IGKJ4*01 | 4 |
| Group 2 | IGHV2-51*01 | IGHJ1*01 | 12 | IGKV1-39*01 | IGKJ1*01 | 8 |
| Group 3 | IGHV2-51*01 | IGHJ3*01 | 13 | IGKV1-9*01 | IGKJ2*01 | 3 |

Antibody sequences from Example 1 and 10× Genomics sequencing data of the same immunized mice described in Example 1 were analyzed to identify additional sequences that belong to the clonotype groups in Table E0. From this analysis, four (4) V_{H} sequences and seven (7) V_{L} sequences were selected for Group 1; eight (8) V_{H} sequences and fourteen (14) V_{L} sequences were selected for Group 2, and five (5) V_{H} sequences and eight (8) V_{L} sequences were selected for Group 3.

DNA encoding the selected V_{H} and V_{L} sequences described above, in addition to all the VH and VL sequences of the reference antibodies, were synthesized and cloned into IgG expression vectors. These plasmids were combinatorially co-transfected to generate IgG protein for every permutation of V_{H} and V_{L} sequence within a given clonotype group. This resulted in a total of 528 IgG proteins which were tested for binding to human CD19/CD81 CHOK1 and cyno CD19/CD81 CHOK1 cells using flow cytometry. Based on the flow cytometry and sequence results (data not shown), seven (7) antibody sequences were selected to move forward for further testing.

### Example 3: Flow Cytometry Analysis of Anti-CD19 Antibodies Binding to CD19-Expressing Cell Lines

The anti-CD19 antibodies described in Examples 1 and 2 were studied to determine their binding characteristics with respect to human CD19, as well as their ability to cross-react with cynomolgus (cyno) CD19, as compared to a CD19 binding control. Approximately 200,000 viable cells from cell lines expressing human CD19 (K562:huCD19 and huCD19/CD81 CHOK1) or cyno CD19 (cyCD19/CD81 CHOK1) were seeded in each well of 96-well U-bottom plates (Corning; Catalog No. 3799). Cells were washed twice with ice cold PBS and resuspended in 100 μL of live/dead staining buffer (Invitrogen; Catalog No. 34964A). The cells were then incubated in the dark at room temperature (RT) for 15 minutes. A volume (200 μL) of PBS (Gibco; Catalog No. 14190-144) was added in each well and the cells were washed 3 times using centrifugation.

Meanwhile, separate antibody dilutions of anti-CD19 FMC63 (Sommermeyer et al., Leukemia 31, 2191-2199 (2017); used here as a CD19 binding control; referred to herein interchangeably as “FMC63”), in addition to the anti-CD19 antibodies described in Examples 1 and 2, were prepared in separate 96-well V-bottom plates. A volume (20 μL) of 1 μM antibodies were aliquoted and mixed in 180 μL of a staining buffer (Thermo Fisher; Catalog No. 00-4222-26) to obtain a final concentration of 100 nM of each antibody. The solutions were then thoroughly mixed using a multi-channel pipette, and subsequently 50 μL of a 100 nM dilution was added into 150 μL of staining buffer in a separate 96 well V-bottom plate to obtain 25 nM final concentration of each antibody.

After washing off the live/dead marker dye, the cells were re-suspended in 100 μL of either 100 nM or 25 nM antibody dilution. The cells were then incubated on ice for 30 minutes in the dark. After incubation, 200 μL of ice-cold PBS was added to each well and cells were centrifuged. The PBS washing was repeated two more times using 300 μL of ice-cold PBS.

Meanwhile, a 1:300 dilution of APC-goat anti hu-Fc secondary antibody solution (Jackson Labs; Catalog No. 109-136-088) was prepared in staining buffer (Thermo Fisher; Catalog No. 00-4222-26). After washing, the cells were resuspended in 100 μl of secondary antibody solution and the plate was subsequently incubated on ice in the dark for 30 minutes. The cells were then washed (3×) with 200 μL of ice-cold PBS and 75 μL of fixing buffer (Biolegend; Catalog No. 420801) was added to each well. The cells were then incubated for 15 minutes in the dark at RT and 125 μL of PBS was added to each well to make the final total volume 200 μL. The cells were subsequently analyzed using a FACS Canto 11 flow cytometry system. Data was collected and analyzed using FlowJo software. Based on these 2-point FACS data, ten (10) anti-CD19 antibodies of interest were identified (Table E1). Data showed that the CD19 binders showed binding to human and cyno CD19 expressing cell lines, albeit at different levels (Table E1). While all the tested antibodies demonstrated binding to the two human cell lines; three antibodies, specifically, FMC63, 4-CD19 and 5-CD19, did not show specific binding to cyno CD19 expressing cells (Table E1).

TABLE E1
Two-point FACS Binding Data for the Generated
Anti-CD19 Antibodies and FCM3

| | Binding huCD19 | Binding | Binding cyCD19 |
|---|---|---|---|
| | (CHO | huCD19 | (CHO |
| mAb | CD19/CD81) | (K562 CD19) | CD19/CD81) |
| FMC63 | +++ | +++ | − |
| 4-CD19 | +++ | +++ | − |
| 3-CD19 | +++ | +++ | + |
| 2-CD19 | +++ | +++ | + |
| 9-CD19 | +++ | + | + |
| 1-CD19 | +++ | +++ | +++ |
| 8-CD19 | +++ | +++ | +++ |
| 7-CD19 | +++ | +/− | +++ |
| 6-CD19 | +++ | +++ | + |
| 5-CD19 | +/− | + | − |
| 10-CD19 | +++ | +++ | ++ |

“−” = binding not above background
“−/+” = weak binding observable; potentially nonspecific
“+” = significant binding
“+++” = strong binding, equal or greater than positive controls

After obtaining the 2-point FACS data (see Table E1) for the tested anti-CD19 antibodies, these antibodies were further tested for dose dependent binding to huCD19 expressing K562 cells to obtain EC₅₀ of binding. Approximately, 200,000 live K652:huCD19 cells were seeded per well of 96-well U-bottom plates (Corning; Catalog No. 3799). Cells were washed twice with ice cold PBS and resuspended in 100 μL of live/dead staining buffer (Invitrogen; Catalog No. 34964A). The cells were incubated in the dark at room temperature (RT) for 15 minutes. A volume (200 μL) of PBS (Gibco; Catalog No. 14190-144) was added in each well and the cells were washed 3 times using centrifugation.

Meanwhile, serial dilutions of the tested anti-CD19 antibodies (and FMC63 control) were prepared in FACS buffer such that 300 μl of 100 nM highest concentration solution was prepared for each antibody in a V-bottom 96 well plate. The antibodies were serially diluted 1:3 by adding 100 μl of the 100 nM solution to 200 μl of FACS buffer to obtain the serial dilution series (i.e., 100 nM, 33.33 nM, 11.11 nM, 3.70 nM, 1.23 nM, 0.41 nM, and 0 nM [antibody]).

After washing off the live/dead marker dye, the cells were re-suspended in 100 μL of antibody dilution. The cells were incubated on ice for 30 minutes in the dark. After incubation, 200 μL of ice-cold PBS was added to each well and cells were centrifuged. The PBS washing was repeated two more times using 300 μL of ice-cold PBS.

Meanwhile, a 1:300 dilution of APC-goat anti hu-Fc secondary antibody solution (Jackson Labs; Catalog No. 109-136-088) was prepared in staining buffer (Thermo Fisher; Catalog No. 00-4222-26). After washing, the cells were resuspended in 100 μl of secondary antibody solution and the plate was incubated on ice in the dark for 30 minutes. The cells were then washed (3×) with 200 μL of ice-cold PBS and 75 μL of fixing buffer (Biolegend; Catalog No. 420801) was added to each well. The cells were incubated for 15 minutes in the dark at RT and 125 μL of PBS was added to each well to make the final total volume 200 μL. The cells were analyzed using a FACS Canto 11 flow cytometry system. Data was collected and analyzed using FlowJo software.

Data showed that all the tested antibodies bind to the huCD19 expressing K562 cells in a dose dependent manner and the EC₅₀ was calculated for each tested antibody using 3-point non-linear fit using Prism software (Table E2).

TABLE E2
EC₅₀ Data for Dose-dependent Binding of the Generated Anti-CD19
Antibodies and FMC63 to huCD19-expressing K562 cells

| | FACS EC₅₀ |
|---|---|
| mAb | (nM K562) |
| FMC63 | 0.5-5 |
| 4-CD19 | 0.18 |
| 3-CD19 | 0.33 |
| 2-CD19 | 0.36 |
| 9-CD19 | 0.47 |
| 1-CD19 | 0.699 |
| 8-CD19 | 1.4 |
| 7-CD19 | 2.1 |
| 6-CD19 | 2.44 |
| 5-CD19 | 41.1 |
| 10-CD19 | 82.09 |

The heavy chain variable regions (V_{H})s and light chain variable regions (V_{L})s along with the CDRs of the generated anti-CD19 antibodies exemplified here (i.e., 1-CD19 through 10-CD19) are provided below.

### Summary of CD19 Binding Domains

### 1-CD19 Binder:

The heavy chain variable region (VH) amino acid sequence of 1-CD19 is described in SEQ ID NO: 32.

(SEQ ID NO: 32: CDR domains are in bold)
QVQLQQWGAGLLKPSETLSLTCAVYGGSFSDYYWSWVRQPPGKG
LEWIGEITHSGSTNYNPSLKSRVTISVDTSKNQFSLKLSSVTAA
DTAVYYCARGGLDIFTGPYYFDSWGQGALVTVSS

The VH CDR amino acid sequences of 1-CD19 are as follows: DYYWS (VH CDR1; SEQ ID NO: 9); EITHSGSTNYNPSLKS (VH CDR2; SEQ ID NO: 10); GGLDIFTGPYYFDS (VH CDR3; SEQ ID NO: 11).

The light chain variable region (VL) amino acid sequence of 1-CD19 is described in SEQ ID NO: 33.

(SEQ ID NO: 33: CDR domains are in bold)
EIVLTQSPSSLSASVGDRVTITCRASQEIRKDLAWYQQKPGKAP
KVLIYGASSLRSGVPSSFSGSGSGTDFTLTISSLQPEDFATYYC
QQDYNYPPTFGQGTKVEIK

The VL CDR amino acid sequences of 1-CD19 are as follows: RASQEIRKDLA (VL CDR1; SEQ ID NO: 14); GASSLRS (VL CDR2; SEQ ID NO: 15); QQDYNYPPT (VL CDR3; SEQ ID NO: 16).

### 2-CD19 Binder:

The heavy chain variable region (VH) amino acid sequence of 2-CD19 is described in SEQ ID NO: 34.

(SEQ ID NO: 34: CDR domains are in bold)
QVQLVQSGAEVKKPGESLKISCKGSGYSFTSYWIGWVRQMPGKG
LELMGIIYPGDSDTRYSPSFQGQVTISADKSISTAYLQWSSLKA
SDTAMYYCARGGTTIFGVVFEYWGQGTLVTVSS

The VH CDR amino acid sequences of 2-CD19 are as follows: SYWIG (VH CDR1; SEQ ID NO: 118); IIYPGDSDTRYSPSFQG (VH CDR2; SEQ ID NO: 119); GGTTIFGVVFEY (VH CDR3; SEQ ID NO: 120).

The light chain variable region (VL) amino acid sequence of 2-CD19 is described in SEQ ID NO: 35.

(SEQ ID NO: 35: CDR domains are in bold)
EIVLTQSPSSLSTSVGDRVTITCRASQSINNYLNWYQQKPGKAP
KLLIYGASSLQSGVPSRFSGSGSGTDFTLTISSLQPEDFATYYC
QQTYSTPWTFGQGTKVEIK

The VL CDR amino acid sequences of 2-CD19 are as follows:

(VL CDR1; SEQ ID NO: 121)
RASQSINNYLN;
(VL CDR2; SEQ ID NO: 122)
GASSLQS;
(VL CDR3; SEQ ID NO: 123)
QQTYSTPWT.

### 3-CD19 Binder:

The heavy chain variable region (VH) amino acid sequence of 3-CD19 is described in SEQ ID NO: 44.

(SEQ ID NO: 44: CDR domains are in bold)
EVQLVQSGAEVKKPGESLKISCKGSGYSFTSYWIGWVRQMPGKG
LEWMGIIYPGDSDTRYSPSFQGQVTISADKSLSTAYLQWSGLKA
SDTAMYYCARQSIMGVIYVAFDVWGQGTMVTVSS

The VH CDR amino acid sequences of 3-CD19 are as follows: SYWIG (VH CDR1; SEQ ID NO: 118); IIYPGDSDTRYSPSFQG (VH CDR2; SEQ ID NO: 119); QSIMGVIYVAFDV (VH CDR3; SEQ ID NO: 124).

The light chain variable region (VL) amino acid sequence of 3-CD19 is described in SEQ ID NO: 45.

(SEQ ID NO: 45: CDR domains are in bold)
EIVLTQSPSSLSASVGDRVTITCRASQSISSYLNWYQQKPGKAP
KLLIYAASSLQSGVPSRFSGSGSGTDFTLTITSLRPEDFATYYC
QQSYSNPWTFGQGTKVEIK

The VL CDR amino acid sequences of 3-CD19 are as follows:

(VL CDR1; SEQ ID NO: 125)
RASQSISSYLN;
(VL CDR2; SEQ ID NO: 126)
AASSLQS;
(VL CDR3; SEQ ID NO: 127)
QQSYSNPWT.

### 4-CD19 Binder:

The heavy chain variable region (VH) amino acid sequence of 4-CD19 is described in SEQ ID NO: 46.

### (SEQ ID NO: 46: CDR Domains are in Bold)

EVQLVQSGAEVKKPGESLKISCKGSGYSFTSYWIGWVRQMPGKG
LEWMGIIYPGDSDTRYSPSFQGQVTISADKSISTAYLQWSSLKA
SDTAMYYCARGGATIFGVVFDYWGQGTLVTVSS

The VH CDR amino acid sequences of 4-CD19 are as follows: SYWIG (VH CDR1; SEQ ID NO: 118); IIYPGDSDTRYSPSFQG (VH CDR2; SEQ ID NO: 119); GGATIFGVVFDY (VH CDR3; SEQ ID NO: 128).

The light chain variable region (VL) amino acid sequence of 4-CD19 is described in SEQ ID NO: 47.

(SEQ ID NO: 47: CDR domains are in bold)
EIVLTQSPSSLSASVGDRVTITCRASQSINSYLSWFQQKPGKAP
KLLIYAASSLQSGVPSRFSGSGSGTDFTLTISSLQPEDFATYYC
QQSYSTPPTFGQGTKVEIK

The VL CDR amino acid sequences of 4-CD19 are as follows:

(VL CDR1; SEQ ID NO: 129)
RASQSINSYLS;
(VL CDR2; SEQ ID NO: 126)
AASSLQS;
(VL CDR3; SEQ ID NO: 130)
QQSYSTPPT.

### 5-CD19 Binder:

The heavy chain variable region (VH) amino acid sequence of 5-CD19 is described in SEQ ID NO: 48.

(SEQ ID NO: 48: CDR domains are in bold)
QVQLQESGPGLVKPSETLSLTCTVSGGSISSYYWSWIRQPPGKG
LEWIGYIYYSGSTKYNPSLKSRVTISVDTSKNQFSLQLSSVTAA
DTAVYYCATGLAAAGDYYYGMDVWGQGTTVTVSS

The VH CDR amino acid sequences of 5-CD19 are as follows: SYYWS (VH CDR1; SEQ ID NO: 131); YIYYSGSTKYNPSLKS (VH CDR2; SEQ ID NO: 132); GLAAAGDYYYGMDV (VH CDR3; SEQ ID NO: 133).

The light chain variable region (VL) amino acid sequence of 5-CD19 is described in SEQ ID NO: 49.

(SEQ ID NO: 49: CDR domains are in bold)
EIVLTQSPSSLSASVGDRVTITCRASQGISSALAWYQQKPGKAP
KLLIYDASSLESGVPSRFSGGGSGTDFTLTISSLQPEDFASYYC
QQFNSYPLTFGGGTKVEIK

The VL CDR amino acid sequences of 5-CD19 are as follows:

(VL CDR1; SEQ ID NO: 134)
RASQGISSALA;
(VL CDR2; SEQ ID NO: 25)
DASSLES;
(VL CDR3; SEQ ID NO: 135)
QQFNSYPLT.

### 6-CD19 Binder:

The heavy chain variable region (VH) amino acid sequence of 6-CD19 is described in SEQ ID NO: 50.

(SEQ ID NO: 50: CDR domains are in bold)
QVQLVQSGAEVKKPGESLKISCKGSGYSFTSYWIGWVRQMPGKGLEWM
GIIYPGDSDTRYSPSFQGQVTISADKSISTAYLQWSSLKASDTAMYYCAR
QSIVATITWAFDIWGQGTMVTVSS

The VH CDR amino acid sequences of 6-CD19 are as follows: SYWIG (VH CDR1; SEQ ID NO: 118); IIYPGDSDTRYSPSFQG (VH CDR2; SEQ ID NO: 119); QSIVATITWAFDI (VH CDR3; SEQ ID NO: 136).

The light chain variable region (VL) amino acid sequence of 6-CD19 is described in SEQ ID NO: 51.

(SEQ ID NO: 51: CDR domains are in bold)
EIVLTQSPSFLSASVGDRVTITCRASQGISSYLAWYQQKPGKAPKLLIYA
ASTLQSGVPSRFSGSGSGTEFTLTVSSLQPEDFATYYCQQLNSYPYTFGQ
GTKLEIK

The VL CDR amino acid sequences of 6-CD19 are as follows: RASQGISSYLA (VL CDR1; SEQ ID NO: 137); AASTLQS (VL CDR2; SEQ ID NO: 138); QQLNSYPYT (VL CDR3; SEQ ID NO: 139).

### 7-CD19 Binder:

The heavy chain variable region (VH) amino acid sequence of 7-CD19 is described in SEQ ID NO: 52.

(SEQ ID NO: 52: CDR domains are in bold)
QVQLQESGPGLVKPSETLSLTCTVSGGSISSGSYNWGWIRQPPGKGLEWI
GSIYYSGSTYYNPSLKSRVTISGDTSKNQFSLKLSSLTAADTAVYYCARH
TGGFDYWGQGTLVTVSS

The VH CDR amino acid sequences of 7-CD19 are as follows: SGSYNWG (VH CDR1; SEQ ID NO: 140); SIYYSGSTYYNPSLKS (VH CDR2; SEQ ID NO: 141); HTGGFDY (VH CDR3; SEQ ID NO: 142).

The light chain variable region (VL) amino acid sequence of 7-CD19 is described in SEQ ID NO: 53.

(SEQ ID NO: 53: CDR domains are in bold)
EIVLTQSPATLSVSPGERATLSCRASQSVSSNLAWYQQKPGQAPRLLIYG
ASIRATGIPARFSGSGSGTEFTLTISSLQSEDFAVYYCQQYNNWPLTFGG
GTKVEIK

The VL CDR amino acid sequences of 7-CD19 are as follows:

(VL CDR1; SEQ ID NO: 143)
RASQSVSSNLA;
(VL CDR2; SEQ ID NO: 144)
GASIRAT;
(VL CDR3; SEQ ID NO: 145)
QQYNNWPLT.

### 8-CD19 Binder:

The heavy chain variable region (VH) amino acid sequence of 8-CD19 is described in SEQ ID NO: 54.

(SEQ ID NO: 54: CDR domains are in bold)
QVQLVQSGAEVKKPGESLRISCKGSGYSFSSYWIAWVRQMPGKGLEWM
GIIYPGDSDTRYSPSFQGQVTISADKSISTAYLQWSSLKASDTAMYYCARH
GGYNYGFTDYYYGMDVWGQGTTVTVSS

The VH CDR amino acid sequences of 8-CD19 are as follows: SYWIA (VH CDR1; SEQ ID NO: 146); IIYPGDSDTRYSPSFQG (VH CDR2; SEQ ID NO: 119); HGGYNYGFTDYYYGMDV (VH CDR3; SEQ ID NO: 147).

The light chain variable region (VL) amino acid sequence of 8-CD19 is described in SEQ ID NO: 55.

(SEQ ID NO: 55: CDR domains are in bold)
DIVMTQTPLSSPVTLGQPASISCRSSQRLVHSDGDTYLSWLQQRPGQPPR
LLIYKISNRFSGVPDRFSGSGAGTDFTLKISRVEAEDVGVYYCMQATQFPY
TFGQGTKLEIK

The VL CDR amino acid sequences of 8-CD19 are as follows:

(VL CDR1; SEQ ID NO: 148)
RSSQRLVHSDGDTYLS;
(VL CDR2; SEQ ID NO: 149)
KISNRFS;
(VL CDR3; SEQ ID NO: 150)
MQATQFPYT.

### 9-CD19 Binder:

The heavy chain variable region (VH) amino acid sequence of 9-CD19 is described in SEQ ID NO: 56.

(SEQ ID NO: 56: CDR domains are in bold)
EVQLVQSGAEVKKPGESLKISCKGSGYSFTSYWIGWVRQMPGKGLEWM
GIIYPGDSDTRYSPSFQGQVAISADKSISTAYLQWSSLKASDTAMYYCARR
SIAGTTSWSFDLWGRGTLVTVSS

The VH CDR amino acid sequences of 9-CD19 are as follows: SYWIG (VH CDR1; SEQ ID NO: 118); IIYPGDSDTRYSPSFQG (VH CDR2; SEQ ID NO: 119); RSIAGTTSWSFDL (VH CDR3; SEQ ID NO: 151).

The light chain variable region (VL) amino acid sequence of 9-CD19 is described in SEQ ID NO: 57.

(SEQ ID NO: 57: CDR domains are in bold)
EIVLTQSPSSLSASVGDRVTITCRTSQDIRNDLGWYQQKPGNAPKLLIYA
ASSLQSGVPSRFSGSGSGTDFTLTISSLQPEDFATYYCLQDYNYPLTFGG
GTKVEIK

The VL CDR amino acid sequences of 9-CD19 are as follows:

(VL CDR1; SEQ ID NO: 152)
RTSQDIRNDLG;
(VL CDR2; SEQ ID NO: 126)
AASSLQS;
(VL CDR3; SEQ ID NO: 153)
LQDYNYPLT.

### 10-CD19 Binder:

The heavy chain variable region (VH) amino acid sequence of 10-CD19 is described in SEQ ID NO: 58.

(SEQ ID NO: 58: CDR domains are in bold)
QVQLQESGPGLVKPSETLSLTCTVSGGSFSSYYWSWIRQPPGKGLEWIGY
IYYSGRTNYNPSLKSRVTISIDTSKNQVSLKLSSVTAADTAVYYCASHHW
GSALDYWGQGTLVTVSS

The VH CDR amino acid sequences of 10-CD19 are as follows: SYYWS (VH CDR1; SEQ ID NO: 131); YIYYSGRTNYNPSLKS (VH CDR2; SEQ ID NO: 154); HHWGSALDY (VH CDR3; SEQ ID NO: 155).

The light chain variable region (VL) amino acid sequence of 10-CD19 is described in SEQ ID NO: 59.

(SEQ ID NO: 59: CDR domains are in bold)
EIVLTQSPLSLPVTLGQPASISCRSSQSLVYSDGNTYLNWFQQRPGQSPR
RLIYKVSNRDSGVPDRFSGSGSGTDFTLKISRVEAEDVGVYYCMQGTHWP
WTFGQGTKVEIK

The VL CDR amino acid sequences of 10-CD19 are as follows:

(VL CDR1; SEQ ID NO: 156);
RSSQSLVYSDGNTYLN
(VL CDR2; SEQ ID NO: 157)
KVSNRDS;
(VL CDR3; SEQ ID NO: 158)
MQGTHWPWT.

These studies identified CD19 binders that bound to human CD19-expressing cells, with several also demonstrating binding to cynomolgus CD19-expressing cells.

### Example 4: Generation of Anti-CD3 Antibodies

Fully human anti-CD3 antibodies were generated by immunizing human IgG transgenic Kirin-Medarex (KM) mice as described in Lonberg et al. (1994) Nature 368(6474):856-9. Enrichment for antibodies with cross-reactivity to both human and cynomologus (cyno) CD3-epsilon was achieved by footpad immunization of KM mice with a Keyhole limpet hemocyanin-fused N-terminal 14-amino acid peptide of CD3-epsilon that shares 13/14 amino acids between human and cyno CD3-epsilon (i.e., ZDGNEEMGGITQTP (SEQ ID NO:240), where Z represents pyroglutamic acid (PCA), a chemically modified version of the amino acid glutamine (“Q”)). The specific antibody response against CD3 -epsilon in these mice was monitored over time by ELISA-based screening of serum, and mice with sufficient titers of anti-CD3-epsilon antibodies were selected for subsequent fusions. Lymph nodes and spleens from these mice were harvested, and hybridomas were generated via an electroporation unit to provide a pulse of electricity to increase permeability of the cell membrane, allowing for fusion of antibody secreting plasma cells with immortalized myeloma cells.

### Example 5: Identification of Anti-CD3-Epsilon-Specific Antibodies

Hybridoma supernatants from the hybridomas generated in Example 4 were screened by ELISA to identify antibodies specific for the immunizing peptide (i.e., ZDGNEEMGGITQTP (SEQ ID NO:240)). A total of 87 hybridoma fusions were performed, which yielded more than 1000 antibody clones with specificity for CD3-epsilon. ELISA-positive clones were subsequently screened by flow-cytometry for cell-binding to endogenously expressed human and cyno CD3 on immortalized Jurkat cells, primary human pan-T cells (isolated from peripheral blood mononuclear cells from healthy donors), and cyno pan-T cells (isolated from peripheral blood mononuclear cells). Binding of antibodies to cell surface CD3-epsilon was measured with a fluorescently-conjugated secondary anti-human IgG antibody (data not shown). Relative cell binding was measured as mean-fluorescent intensity and compared to a positive control anti-human/cyno cross-reactive anti-CD3 antibody (data not shown). Secondary Fluorescence-activated cell sorting (FACS) analysis yielded approximately 475 clones that bound to membrane-expressed human and cyno CD3 -epsilon. FACS-positive clones were subsequently purified from hybridoma supernatants for functional characterization.

### Example 6: Functional Characterization of Anti-CD3 Antibodies

The ELISA and FACS-positive anti-CD3 clones identified in Example 5 were analyzed to determine their ability to induce proliferation of primary human and cyno pan-T cells. Antibodies were immobilized on wells of 96-well non-tissue culture treated flat-bottom plates at concentrations between 0.1 and 10 μg/milliliter in phosphate buffered saline by overnight incubation at 4° Celsius. Unbound antibody was subsequently washed away, and 1×10⁵ purified primary human or cyno T cells were added directly to antibody-coated wells in 200 μl of RMPI-1640 media containing 10% fetal bovine serum. T-cell proliferation was measured after 72 hours by incubation of cells with 0.5 mCi/well of ³H-thymidine for the final 6 hours of culture. Cells in each well were then lysed, and DNA was captured onto a glass-fiber filter. T-cell proliferation was determined by measuring the amount of radioactive thymidine incorporated into the DNA of the cells (data not shown). This functional analysis identified a total of 32 clones that were able to induce proliferation of primary human and cyno T cells with similar potency to a positive control anti-CD3 cyno/human cross-reactive (SP34), or at least 1.5-fold increase in proliferation compared to an isotype control antibody (data not shown).

### Example 7: Binding Affinity of Anti-CD3 Antibodies to Human and Cyno CD3 Determined by Surface Plasmon Resonance (SPR)

The functionally active anti-CD3 antibodies identified in Example 6 were sequenced by next-generation sequencing and then produced recombinantly in HEK cells by transient expression in HEK cells. The binding affinities and kinetic constants of the recombinantly produced antibodies for human CD3 and cyno CD3-epsilon were then determined by surface plasmon resonance (SPR) at 37° C. using an antibody-capture format, multi-cycle kinetics on a Biacore T200 instrument.

A Biacore CM4 Series S sensor chip (Cytiva) was selected for kinetic measurements, and the system was primed with HBS-T+ (10 mM HEPES, 150 mM NaCl, 0.05% Tween-20, pH 7.4 buffer (Corning)) for immobilization. The sensor temperature was adjusted to 37° C. prior to chip insertion, and the sample compartment was maintained at 15° C. Upon priming, the chip was activated using a fresh 1:1 mixture of 0.4 M EDC and 0.1 M NHS (Cytiva) for seven minutes at 20 μl/ml flow rate across all four flow cells. Goat F(ab′)2 anti-human Kappa or goat anti-human IgG Fc polyclonal antibodies (Southern Biotech) were diluted to 60 μg/mL in 10 mM sodium acetate, pH 4.5 (Cytiva) and amine-coupled to all flow cells for 7 minutes at 20 μL/min. Finally, to block the activated chip surface, 0.1 M ethylenediamine in 150 mM sodium borate, pH 8.5 buffer was injected over all flow cells for 7 minutes at 20 μl/minute. Surfaces were regenerated with two 30-second injections of 0.425% phosphoric acid (Sigma-Aldrich) at 30 μL/min.

Anti-CD3 antibodies were measured for their binding affinity and kinetics to human and cyno CD3εδ-muFcG1 in a multi-cycle capture kinetics experiment. Running buffer was switched to HBS-T+ (10 mM HEPES, 150 mM NaCl, 0.05% Tween-20, pH 7.4 buffer (Corning) with 0.1% BSA (Sigma-Aldrich) and the system was thoroughly primed in this buffer. Anti-CD3 antibodies were diluted to 1-10 μg/mL into running buffer and served as ligand. Human and cyno CD3εδ-muFcG1 proteins were serially diluted to 20 nM, 100 nM, and 500 nM concentrations with running buffer. Three startup cycles, which are abbreviated analysis cycles of shortened injections but with complete regeneration, were introduced prior to the analysis experiment. In each analysis cycle, individual anti-CD3 antibodies were separately captured as ligand to flow cells 2, 3 or 4 at 10 μL/min for 30 to 90 seconds to achieve an Rₘₐₓ of approximately 50 RUs. Flow cell 1 remained unaddressed and served as the reference surface. A one-minute stabilization period was introduced after the last ligand capture onto flow cell 4. A single analyte sample was injected per cycle over all four flow cells for 2 min at 30 μL/min with 5-minute dissociation. Analyte samples comprised of the human and cyno CD3εδ-muFcG1 serial dilutions and were injected in order from lowest to highest concentration with buffer blanks flanking each concentration series. Amine-coupled goat F(ab′)2 anti-human Kappa or goat anti-human IgG Fc polyclonal antibody surfaces were regenerated at the end of each cycle with two 30-second injections of 0.425% phosphoric acid (Sigma-Aldrich) at 30 μL/min. Cycles were repeated until every antibody was tested for binding to every analyte sample at all concentrations including buffer blanks.

Finally, the data was analyzed using Biacore T200 evaluation software. The rate of complex formation (kₐ) and dissociation (k_{d}) as well as overall dissociation constant (K_{D}) for a selected anti-CD3 antibody are shown in Table E3. The V_{H} and V_{L} sequences of the parental anti-CD3 antibody, i.e., 1-CD3, are shown in Table E4.

TABLE E3
Kinetics and affinity of binding of the parental anti-CD3
antibody 1-CD3

| Antibody | Antigen | kₐ (M⁻¹s⁻¹) | k_{d} (s⁻¹) | K_{D} (nM) |
|---|---|---|---|---|
| 1-CD3 | human CD3- | 3.6E+05 | 2.9E−03 | 8.3 |
| | epsilon | | | |
| 1-CD3 | cyno CD3- | 4.5E+05 | 3.2E−03 | 7.1 |
| | epsilon | | | |

TABLE E4
V_{H}/V_{L} Sequences of the
parental anti-CD3 antibody 1-CD3

| | | SEQ |
|---|---|---|
| Description | Sequence | ID NO: |
| 1-CD3 | QVQLVESGGGVVQPGRSLRLSCA | 36 |
| Variable | ASGFTFSRYGIHWVRQAPGKGLE | |
| heavy (VH) | WVAAIWYDGSKKYYADSVKGRFT | |
| | ISRDNSKNTLDLQMNSLRAEDTA | |
| | VYYCARSNYDILTRYYQYGINVW | |
| | GQGTTVTVSS | |
| 1-CD3 | AIQLTQSPSSLSASVGDRVTITC | 37 |
| Variable | RASQGISSALAWYQQKPGKAPKL | |
| light (VL) | LIYDASSLENGVPSRFSGSGSGT | |
| | DFTLTISSLQPEDFATYYCQQFK | |
| | SYPRTFGQGTKVEIK | |

### Example 8: Engineering the Affinity of the CD3 Binding Arm of Anti-CD3 Antibodies to Modulate Binding Activity

Previous work in mice has shown that anti-CD3-bispecific antibodies with high affinity (<1 nM) for CD3-epsilon accumulate primarily in secondary lymphoid organs, where most T cells reside (Mandikian et. al (2018) Mol Cancer Ther. 17(4):776-785).

To generate affinity engineered CD3 binders, residues involved in CD3 antigen-binding were identified by comparing structural models of the 1-CD3 parental antibody with structural models of clones with sequences similar to the 1-CD3 sequence but having lower affinity binding to CD3 (identified in Example 5, data not shown). The residues identified in this analysis were then substituted to produce seventeen (17) heavy chain variants (identified as 1-CD3 M1-M3, M5-M11, and M16-M22) and five (5) light chain variants (identified as 1-CD3 M4 and M12-M15) having modulated affinity for CD3. Framework mutations in these modulated affinity variants were subsequently converted back to germline sequence to produce 1-CD3 heavy chain variants C1, C5, C6, C8, C9, C11, C16, C17, C21, and C22 and light chain variant C12. The sequences of the affinity variant binders and framework fixed chains are shown in Table E5 (V_{H} sequences) and Table E6 (V_{L} sequences).

TABLE E5
Sequences of 1-CD3-VH variants having modulated affinity

| Description | Amino acid Sequence | SEQ ID NO: |
|---|---|---|
| 1-CD3-M1 | QVQLVESGGGVVQPGRSLRLSCAASGFTFSRYGIHWVRQAPGKG | 41 |
| | LEWVAAIWYDGSKKYYADSVKGRFTISRDNSKNTLYLQMNSLR | |
| | AEDTAVYYCARSNYDILTRYYQYGINVWGQGTTVTVSS | |
| 1-CD3-M2 | QVQLVESGGGVVQPGRSLRLSCAASGFTFSRYGIHWVRQAPGKG | 64 |
| | LEWVAAIWYDSSKKYYADSVKGRFTISRDNSKNTLDLQMNSLRA | |
| | EDTAVYYCARSNYDILTRYYQYGINVWGQGTTVTVSS | |
| 1-CD3-M3 | QVQLVESGGGVVQPGRSLRLSCAASGFTFSRYGIHWVRQAPGKG | 65 |
| | LEWVAAIWYDASKKYYADSVKGRFTISRDNSKNTLDLQMNSLR | |
| | AEDTAVYYCARSNYDILTRYYQYGINVWGQGTTVTVSS | |
| 1-CD3-M5 | QVQLVESGGGVVQPGRSLRLSCAASGFTFSRYGIHWVRQAPGKG | 66 |
| | LEWVAAIWYDGSHKYYADSVKGRFTISRDNSKNTLDLQMNSLR | |
| | AEDTAVYYCARSNYDILTRYYQYGINVWGQGTTVTVSS | |
| 1-CD3-M6 | QVQLVESGGGVVQPGRSLRLSCAASGFTFSRYGIHWVRQAPGKG | 67 |
| | LEWVAAIWYDGSNKYYADSVKGRFTISRDNSKNTLDLQMNSLR | |
| | AEDTAVYYCARSNYDILTRYYQYGINVWGQGTTVTVSS | |
| 1-CD3-M7 | QVQLVESGGGVVQPGRSLRLSCAASGFTFSRYGIHWVRQAPGKG | 69 |
| | LEWVAAIWYDGSKKYYADSVKGRFTISRDNSKNTLDLQMNSLR | |
| | AEDTAVYYCARSNYDILTRHYQYGINVWGQGTTVTVSS | |
| 1-CD3-M8 | QVQLVESGGGVVQPGRSLRLSCAASGFTFSRYGIHWVRQAPGKG | 71 |
| | LEWVAAIWYDGSKKYYADSVKGRFTISRDNSKNTLDLQMNSLR | |
| | AEDTAVYYCARSNYDILTGHYQYGINVWGQGTTVTVSS | |
| 1-CD3-M9 | QVQLVESGGGVVQPGRSLRLSCAASGFTFSRYGIHWVRQAPGKG | 75 |
| | LEWVAAIWYDGSKKYYADSVKGRFTISRDNSKNTLDLQMNSLR | |
| | AEDTAVYYCARSNYDILTGYYQYGINVWGQGTTVTVSS | |
| 1-CD3- | QVQLVESGGGVVQPGRSLRLSCAASGFTFSRYGIHWVRQAPGKG | 76 |
| M10 | LEWVAAIWYDGSKKYYADSVKGRFTISRDNSKNTLDLQMNSLR | |
| | AEDTAVYYCARSNYDILTRYYQHGINVWGQGTTVTVSS | |
| 1-CD3- | QVQLVESGGGVVQPGRSLRLSCAASGFTFSRYGIHWVRQAPGKG | 77 |
| M11 | LEWVAAIWYDGSKKYYADSVKGRFTISRDNSKNTLDLQMNSLR | |
| | AEDTAVYYCARSNYDILTRYYSHGINVWGQGTTVTVSS | |
| 1-CD3- | QVQLVESGGGVVQPGRSLRLSCAASGFTFSNYGIHWVRQAPGKG | 79 |
| M16 | LEWVAAIWYDGSKKYYADSVKGRFTISRDNSKNTLDLQMNSLR | |
| | AEDTAVYYCARSNYDILTRYYQYGINVWGQGTTVTVSS | |
| 1-CD3- | QVQLVESGGGVVQPGRSLRLSCAASGFTFSNHGIHWVRQAPGKG | 81 |
| M17 | LEWVAAIWYDGSKKYYADSVKGRFTISRDNSKNTLDLQMNSLR | |
| | AEDTAVYYCARSNYDILTRYYQYGINVWGQGTTVTVSS | |
| 1-CD3- | QVQLVESGGGVVQPGRSLRLSCAASGFTFSRHGIHWVRQAPGKG | 83 |
| M18 | LEWVAAIWYDGSKKYYADSVKGRFTISRDNSKNTLDLQMNSLR | |
| | AEDTAVYYCARSNYDILTRYYQYGINVWGQGTTVTVSS | |
| 1-CD3- | QVQLVESGGGVVQPGRSLRLSCAASGFTFSRYGIHWVRQAPGKG | 85 |
| M19 | LEWVAAIWYDGSKKYYADSVKGRFTISRDNSKNTLDLQMNSLR | |
| | AEDTAVYYCARSNFDILTRYYQYGINVWGQGTTVTVSS | |
| 1-CD3- | QVQLVESGGGVVQPGRSLRLSCAASGFTFSRYGIHWVRQAPGKG | 87 |
| M20 | LEWVAAIWYDGSKKYYADSVKGRFTISRDNSKNTLDLQMNSLR | |
| | AEDTAVYYCARSNFDVLTRYYQYGINVWGQGTTVTVSS | |
| 1-CD3- | QVQLVESGGGVVQPGRSLRLSCAASGFTFSRYGIHWVRQAPGKG | 90 |
| M21 | LEWVAAIWYDGSKKYYADSVKGRFTISRDNSKNTLDLQMNSLR | |
| | AEDTAVYYCARSNYDVLTRYYQYGINVWGQGTTVTVSS | |
| 1-CD3- | QVQLVESGGGVVQPGRSLRLSCAASGFTFSRYGIHWVRQAPGKG | 91 |
| M22 | LEWVAAIWYDGSKKYYADSVKGRFTISRDNSKNTLDLQMNSLR | |
| | AEDTAVYYCARSNYDILTRYYQYGLDVWGQGTTVTVSS | |
| 1-CD3-C1 | QVQLVESGGGVVQPGRSLRLSCAASGFTFSRYGIHWVRQAPGKG | 92 |
| | LEWVAAIWYDASKKYYADSVKGRFTISRDNSKNTLYLQMNSLR | |
| | AEDTAVYYCARSNYDILTRYYQYGINVWGQGTTVTVSS | |
| 1-CD3-C5 | QVQLVESGGGVVQPGRSLRLSCAASGFTFSRYGIHWVRQAPGKG | 93 |
| | LEWVAAIWYDASHKYYADSVKGRFTISRDNSKNTLYLQMNSLR | |
| | AEDTAVYYCARSNYDILTRYYQYGINVWGQGTTVTVSS | |
| 1-CD3-C6 | QVQLVESGGGVVQPGRSLRLSCAASGFTFSRYGIHWVRQAPGKG | 94 |
| | LEWVAAIWYDASNKYYADSVKGRFTISRDNSKNTLYLQMNSLR | |
| | AEDTAVYYCARSNYDILTRYYQYGINVWGQGTTVTVSS | |
| 1-CD3-C8 | QVQLVESGGGVVQPGRSLRLSCAASGFTFSRYGIHWVRQAPGKG | 95 |
| | LEWVAAIWYDASKKYYADSVKGRFTISRDNSKNTLYLQMNSLR | |
| | AEDTAVYYCARSNYDILTGHYQYGINVWGQGTTVTVSS | |
| 1-CD3-C9 | QVQLVESGGGVVQPGRSLRLSCAASGFTFSRYGIHWVRQAPGKG | 96 |
| | LEWVAAIWYDASKKYYADSVKGRFTISRDNSKNTLYLQMNSLR | |
| | AEDTAVYYCARSNYDILTGYYQYGINVWGQGTTVTVSS | |
| 1-CD3-C11 | QVQLVESGGGVVQPGRSLRLSCAASGFTFSRYGIHWVRQAPGKG | 42 |
| | LEWVAAIWYDASKKYYADSVKGRFTISRDNSKNTLYLQMNSLR | |
| | AEDTAVYYCARSNYDILTRYYSHGINVWGQGTTVTVSS | |
| 1-CD3-C16 | QVQLVESGGGVVQPGRSLRLSCAASGFTFSNYGIHWVRQAPGKG | 38 |
| | LEWVAAIWYDASKKYYADSVKGRFTISRDNSKNTLYLQMNSLR | |
| | AEDTAVYYCARSNYDILTRYYQYGINVWGQGTTVTVSS | |
| 1-CD3-C17 | QVQLVESGGGVVQPGRSLRLSCAASGFTFSNHGIHWVRQAPGKG | 97 |
| | LEWVAAIWYDASKKYYADSVKGRFTISRDNSKNTLYLQMNSLR | |
| | AEDTAVYYCARSNYDILTRYYQYGINVWGQGTTVTVSS | |
| 1-CD3-C21 | QVQLVESGGGVVQPGRSLRLSCAASGFTFSRYGIHWVRQAPGKG | 98 |
| | LEWVAAIWYDASKKYYADSVKGRFTISRDNSKNTLYLQMNSLR | |
| | AEDTAVYYCARSNYDVLTRYYQYGINVWGQGTTVTVSS | |
| 1-CD3-C22 | QVQLVESGGGVVQPGRSLRLSCAASGFTFSRYGIHWVRQAPGKG | 40 |
| | LEWVAAIWYDASKKYYADSVKGRFTISRDNSKNTLYLQMNSLR | |
| | AEDTAVYYCARSNYDILTRYYQYGLDVWGQGTTVTVSS | |

TABLE E6
Sequences of 1-CD3-VL variants having modulated affinity

| Description | Amino acid Sequence | SEQ ID NO: |
|---|---|---|
| 1-CD3-M4 | AIQLTQSPSSLSASVGDRVTITCRASQGISSALAWYQQKPGKAPK | 39 |
| | LLIYDASSLESGVPSRFSGSGSGTDFTLTISSLQPEDFATYYCQQF | |
| | KSYPRTFGQGTKVEIK | |
| 1-CD3-M12 | AIQLTQSPSSLSASVGDRVTITCRASQGISSALAWYQQKPGKAPK | 99 |
| | LLIYDASSLENGVPSRFSGSGSGTDFTLTISSLQPEDFATYYCQQF | |
| | NSYPRTFGQGTKVEIK | |
| 1-CD3-M13 | AIQLTQSPSSLSASVGDRVTITCRASQGISSALAWYQQKPGKAPK | 43 |
| | LLIYDASSLESGVPSRFSGSGSGTDFTLTISSLQPEDFATYYCQQF | |
| | NSYPRTFGQGTKVEIK | |
| 1-CD3-M14 | AIQLTQSPSSLSASVGDRVTITCRASQGISSALAWYQQKPGKAPK | 100 |
| | LLIYDASGLENGVPSRFSGSGSGTDFTLTISSLQPEDFATYYCQQF | |
| | KSYPRTFGQGTKVEIK | |
| 1-CD3-M15 | AIQLTQSPSSLSASVGDRVTITCRASQGISSALAWYQQKPGKAPK | 101 |
| | LLIYDASGLESGVPSRFSGSGSGTDFTLTISSLQPEDFATYYCQQF | |
| | NSYPRTFGQGTKVEIK | |
| 1-CD3-C12 | AIQLTQSPSSLSASVGDRVTITCRASQGISSALAWYQQKPGKAPK | 43 |
| | LLIYDASSLESGVPSRFSGSGSGTDFTLTISSLQPEDFATYYCQQF | |
| | NSYPRTFGQGTKVEIK | |

Next, a library of anti-CD3 antibodies having a range of anti-CD3 affinities was created by pairing the 18 different heavy chains (wild type 1-CD3 and the 17 heavy chain affinity variants 1-CD3-M1, 1-CD3-M2, 1-CD3-M3, 1-CD3-M5, 1-CD3-M6, 1-CD3-M7, 1-CD3-M8, 1-CD3-M9, 1-CD3-M10, 1-CD3-M11, 1-CD3-M16, 1-CD3-M17, 1-CD3-M18, 1-CD3-M19, 1-CD3-M20, 1-CD3-M21, and 1-CD3-M22 as shown in Table E5) with 6 different light chain (wild type 1-CD3 and 5 affinity variants 1-CD3-M4, 1-CD3-M12, 1-CD3-M13, 1-CD3-M14 and 1-CD3-M15 as shown in Table E6) to create 107 variants of the 1-CD3 parental antibody. These engineered 1-CD3 antibody variants were expressed recombinantly, and the affinity of each antibody to human and/or cyno CD3Γδ-muFcG1 was assessed by SPR as described in the Working Examples disclosed herein, e.g., Example 7. The binding affinity for the 107 1-CD3 variants ranged from 5 nM to 150 nM.

In order to select the CD3 binding arm with the most favorable affinity profile, 1-CD3 variants having a range of affinities from 10 to 70 nM were selected from the library described above plus incorporation of the framework fixed chains, and the affinities are shown in Table E7.

TABLE E7
CD3 affinities of 1-CD3 wild type and affinity variant antibodies

| Antibody | CD3 affinity K_{D} (nM) |
|---|---|
| 1-CD3 (parental) | 7 |
| 2-CD3 | 11 |
| 3-CD3 | 5 |
| 4-CD3 | 21 |

A summary of the CD3 binding domains (1-CD3, 2-CD3, 3-CD3 and 4-CD3) is provided below.

### 1-CD3 Binder

The heavy chain variable region (VH) amino acid sequence of 1-CD3 is described in SEQ ID NO: 36.

(SEQ ID NO: 36; CDR domains are in bold)
QVQLVESGGGVVQPGRSLRLSCAASGFTFSRYGIHWVRQAPGKGLEWVA
AIWYDGSKKYYADSVKGRFTISRDNSKNTLDLQMNSLRAEDTAVYYCAR
SNYDILTRYYQYGINVWGQGTTVTVSS

The VH CDR amino acid sequences of 1-CD3 are as follows: RYGIH (VH CDR1; SEQ ID NO: 19); AIWYDGSKKYYADSVKG (VH CDR2; SEQ ID NO: 107); SNYDILTRYYQYGINV (VH CDR3; SEQ ID NO: 108).

The light chain variable region (VL) amino acid sequence of 1-CD3 is described in SEQ ID NO: 37.

(SEQ ID NO: 37: CDR domains are in bold)
AIQLTQSPSSLSASVGDRVTITCRASQGISSALAWYQQKPGKAPKLLIY
DASSLENGVPSRFSGSGSGTDFTLTISSLQPEDFATYYCQQFKSYPRTF
GQGTKVEIK

The VL CDR amino acid sequences of 1-CD3 are as follows: RASQGISSALA (VL CDR1; SEQ ID NO: 24); DASSLEN (VL CDR2; SEQ ID NO: 114); QQFKSYPRT (VL CDR3; SEQ ID NO: 26).

### 2-CD3 Binder

The heavy chain variable region (VH) amino acid sequence of 2-CD3 is described in SEQ ID NO: 38 (note: VH amino acid changes when compared to the VH of 1-CD3 binder (i.e., SEQ ID NO: 36) are R31N, G55A, D80Y)

(SEQ ID NO: 38; CDR domains are in bold)
QVQLVESGGGVVQPGRSLRLSCAASGFTFSNYGIHWVRQAPGKGLEWVA
AIWYDASKKYYADSVKGRFTISRDNSKNTLYLQMNSLRAEDTAVYYCAR
SNYDILTRYYQYGINVWGQGTTVTVSS

The VH CDR amino acid sequences of 2-CD3 are as follows: NYGIH (VH CDR1; SEQ ID NO: 109); AIWYDASKKYYADSVKG (VH CDR2; SEQ ID NO: 110); SNYDILTRYYQYGINV (VH CDR3; SEQ ID NO: 108).

The light chain variable region (VL) amino acid sequence of 2-CD3 is described in SEQ ID NO: 39 (note: VL amino acid change when compared to the VL of 1-CD3 binder (i.e., SEQ ID NO: 37) is N56S).

(SEQ ID NO: 39: CDR domains are in bold)
AIQLTQSPSSLSASVGDRVTITCRASQGISSALAWYQQKPGKAPKLLIY
DASSLESGVPSRFSGSGSGTDFTLTISSLQPEDFATYYCQQFKSYPRTF
GQGTKVEIK

The VL CDR amino acid sequences of 2-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 3-CD3 Binder

The heavy chain variable region (VH) amino acid sequence of 3-CD3 is described in SEQ ID NO: 40 (note: VH amino acid changes when compared to the VH of 1-CD3 binder (i.e., SEQ ID NO: 36) are G55A, D80Y, I112L, N113D).

(SEQ ID NO: 40; CDR domains are in bold)
QVQLVESGGGVVQPGRSLRLSCAASGFTFSRYGIHWVRQAPGKGLEWVA
AIWYDASKKYYADSVKGRFTISRDNSKNTLYLQMNSLRAEDTAVYYCAR
SNYDILTRYYQYGLDVWGQGTTVTVSS

The VH CDR amino acid sequences of 3-CD3 are as follows: RYGIH (VH CDR1; SEQ ID NO: 19); AIWYDASKKYYADSVKG (VH CDR2; SEQ ID NO: 110); SNYDILTRYYQYGLDV (VH CDR3; SEQ ID NO: 21).

The light chain variable region (VL) amino acid sequence of 3-CD19 is described in SEQ ID NO: 39 (note: VL amino acid change when compared to the VL of 1-CD3 binder (i.e., SEQ ID NO: 37) is N56S).

(SEQ ID NO: 39: CDR domains are in bold)
AIQLTQSPSSLSASVGDRVTITCRASQGISSALAWYQQKPGKAPKLLIY
DASSLESGVPSRFSGSGSGTDFTLTISSLQPEDFATYYCQQFKSYPRTF
GQGTKVEIK

The VL CDR amino acid sequences of 3-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 4-CD3 Binder

The heavy chain variable region (VH) amino acid sequence of 4-CD3 is described in SEQ ID NO: 42 (note: VH amino acid changes when compared to the VH of 1-CD3 binder (i.e., SEQ ID NO: 36) are G55A, D80Y, Q109S, Y110H)

(SEQ ID NO: 42; CDR domains are in bold)
QVQLVESGGGVVQPGRSLRLSCAASGFTFSRYGIHWVRQAPGKGLEWVA
AIWYDASKKYYADSVKGRFTISRDNSKNTLYLQMNSLRAEDTA VYYCA
RSNYDILTRYYSHGINVWGQGTTVTVSS

The VH CDR amino acid sequences of 4-CD3 are as follows: RYGIH (VH CDR1; SEQ ID NO: 19); AIWYDASKKYYADSVKG (VH CDR2; SEQ ID NO: 110); SNYDILTRYYSHGINV (VH CDR3; SEQ ID NO: 111).

The light chain variable region (VL) amino acid sequence of 4-CD3 is described in SEQ ID NO: 43 (note: VL amino acid changes when compared to the VL of 1-CD3 binder (i.e., SEQ ID NO: 37) are N56S and K92N).

(SEQ ID NO: 43: CDR domains are in bold)
AIQLTQSPSSLSASVGDRVTITCRASQGISSALAWYQQKPGKAPKLLIY
DASSLESGVPSRFSGSGSGTDFTLTISSLQPEDFATYYCQQFNSYPRTF
GQGTKVEIK

The VL CDR amino acid sequences of 4-CD3 are as follows: RASQGISSALA (VL CDR1; SEQ ID NO: 24); DASSLES (VL CDR2; SEQ ID NO: 25); QQFNSYPRT (VL CDR3; SEQ ID NO: 115).

### Example 9: Generation of CD19×CD3 TCEs

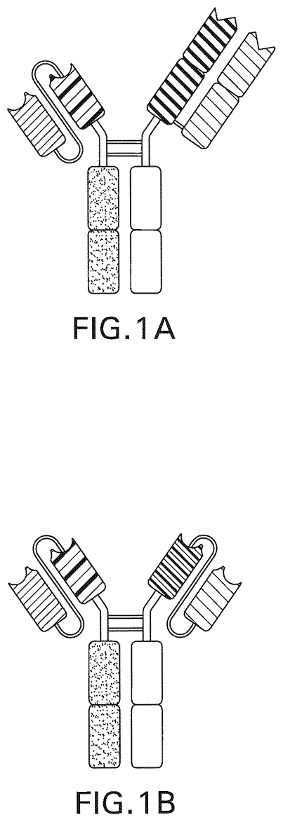

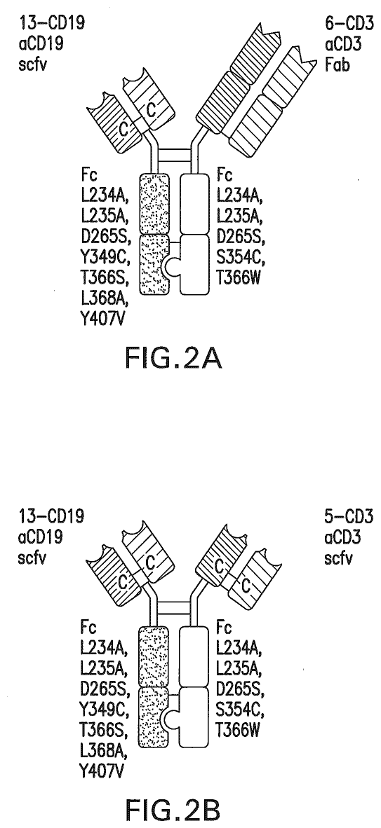

The inventors hypothesized that a CD19×CD3 bispecific antibody, by simultaneously engaging CD19-expressing autoimmune cells (including B cells, plasmablasts, and subsets of plasma cells) and CD3-expressing T cells, would be capable of facilitating the formation of an immunological synapse between B cells and T cells. Such an interaction, the inventors hypothesized, would trigger T cell activation, cytokine production, and subsequent T cell mediated lysis of CD19-expressing target cells, thereby driving a therapeutic mechanism of action. To test this hypothesis, the inventors initially created several CD19×CD3 TCEs in a bispecific IgG format (i.e., Fab-Fc×Fab Fc format). However, analysis of these data revealed suboptimal B cell depletion activity for the CD19×CD3 TCEs in this IgG format (data not shown). The inventors further hypothesized that modulating the structural proximity of the binding arms of a CD19×CD3 bispecific antibody would impact B cell depletion activity (and therefore, therapeutic window) by modulating T cell and B cell proximity to more closely mimic a natural immune synapse. The inventors modeled synapse distances of TCEs where the binder in one or both arms of the TCE were in the scFv instead of Fab format (i.e., the “scFv-Fc×Fab-Fc” format as generally depicted in FIG. 1A or the “scFv-Fc×scFv-Fc” format as generally depicted in FIG. 1B) and found that these distances could more closely mimic natural synapse distances (data not shown). To test this hypothesis, two formats of CD19 TCEs were generated using CD19 binders and CD3 binders where the binder in one or both arms of the TCE were in the scFv instead of Fab format (i.e., the “scFv-Fc×Fab-Fc” format as generally depicted in FIG. 1A or the “scFv-Fc×scFv-Fc” format as generally depicted in FIG. 1B), thereby shortening the distance between the two binding arms and modulating the T cell and B cell proximity. See Table E8 for the amino acid sequences of the component sequences that were used to assemble the CD19 and CD3 binders into TCEs. Exemplary TCEs TCE001 and TCE002 are shown in FIG. 2A and FIG. 2B, respectively. For TCE001, the scFvs for the CD19 binder and CD3 binder were designed using the variable heavy sequence attached via a GGGGSGGGGSGGGGS (SEQ ID NO: 162) linker to the variable light chain which was followed by the hinge sequence of a human IgG1 Fc. See FIG. 2B, Table E8, and Table E9. For TCE002, the scFvs for the CD19 binder was designed using the variable heavy sequence attached via a GGGGSGGGGSGGGGS (SEQ ID NO: 162) linker to the variable light chain which was followed by the hinge sequence of a human IgG1 Fc while the CD3 variable domains were used as Fabs, hence attached to the CH1 or CL domains for the heavy and light chains respectively. See FIG. 2A, Table E8, and Table E9. In order to generate bispecific antibodies combining anti-CD19 and anti-CD3 binding arms, knob-into-hole mutations were introduced into the CH3 region essentially as described in Ridgway et al., Protein Eng. 9(7):617-21 (1996). The Fc of CD19 binding arm of TCE001 and TCE002 was engineered to contain T366S, L368A, and Y407V mutations [Ridgeway Protein Engineering 1996] in addition to an additional disulfide incorporation Y349C (as described in Merchant, et al., Nature Biotechnology. July; 16 (7):677-81 (1998)). The Fc of the CD3 binding arm of TCE001 and TCE002 was engineered to contain the T366W mutation [Ridgeway Protein Engineering 1996] in addition to an additional disulfide incorporation S354C [Merchant Nature Biotechnology 1998]. The Fc region of both TCE001 and TCE002 were additionally engineered to contain effector function reducing mutations L234A, L235A and D265S (“AAS”). See both FIG. 2B and FIG. 2A. One of ordinary skill in the art would understand that alternative Fc regions, such as those described in Table E8 (e.g., Fc-IgG2.3 P238K-Knob (SEQ ID NO: 167) and Fc-IgG2.3 P238K-Hole (Seq ID NO: 168)) can also be used to generate bispecific TCE molecules.

In some instances, the Fc region of one chain of the TCE contained H435R and Y436F mutations to abrogate protein A binding (see Tustian et al., mAbs 8, 828-838 (2016)) in addition to a C-terminal 6His or 10His tag (referred to interchangeably herein as “RFHis modification”). This enabled increased yield of the desired heterodimer following a 1-step Protein A purification or a 2-step purification with anti-His tag capture followed by Protein A. The incorporation of these mutations and tags did not affect the binding of the CD19 TCE to either CD19 or CD3, nor did these mutations affect the ability of the CD19 TCE to deplete target cells (data not shown).

Following small-scale high-throughput production, more than 300 bispecific TCEs were screened by functional characterization of the TCE's ability to deplete targeted cell populations and biophysical assays as described in Examples below.

TABLE E8
Component portions of the TCEs

| | | SEQ ID |
|---|---|---|
| Description | Sequence | NO: |
| G4S linker | GGGGS | 160 |
| (G4S) 2 | GGGGSGGGGS | 161 |
| linker | | |
| (G4S)3 | GGGGSGGGGSGGGGS | 162 |
| linker | | |
| Fc - IgG1.3f- | DKTHTCPPCPAPEAEGAPSVFLFPPKPKDTLMISRTPEVTCVVVDV | 163 |
| Knob (L234A, | SHEDPEVKFNWYVDGVEVHNAKTKPREEQYNSTYRVVSVLTVL | |
| L235E, G237A; | HQDWLNGKEYKCKVSNKALPAPIEKTISKAKGQPREPQVYTLPPC | |
| D356E, L358M; | REEMTKNQVSLWCLVKGFYPSDIAVEWESNGQPENNYKTTPPVL | |
| S354C, T366W) | DSDGSFFLYSKLTVDKSRWQQGNVFSCSVMHEALHNHYTQKSLS | |
| | LSPG | |
| Fc - IgG1.3f- | DKTHTCPPCPAPEAEGAPSVFLFPPKPKDTLMISRTPEVTCVVVDV | 164 |
| Hole (L234A, | SHEDPEVKFNWYVDGVEVHNAKTKPREEQYNSTYRVVSVLTVL | |
| L235E, G237A; | HQDWLNGKEYKCKVSNKALPAPIEKTISKAKGQPREPQVCTLPPS | |
| D356E, L358M; | REEMTKNQVSLSCAVKGFYPSDIAVEWESNGQPENNYKTTPPVL | |
| Y349C, T366S, | DSDGSFFLVSKLTVDKSRWQQGNVFSCSVMHEALHNHYTQKSLS | |
| L368A, Y407V) | LSPG | |
| Fc - IgGI | DKTHTCPPCPAPEAAGGPSVFLFPPKPKDTLMISRTPEVTCVVVSV | 165 |
| KEMA AAS- | SHEDPEVKFNWYVDGVEVHNAKTKPREEQYNSTYRVVSVLTVL | |
| Knob (L234A, | HQDWLNGKEYKCKVSNKALPAPIEKTISKAKGQPREPQVYTLPPC | |
| L235A, D265S; | REEMTKNQVSLWCLVKGFYPSDIAVEWESNGQPENNYKTTPPVL | |
| D356E, L358M; | DSDGSFFLYSKLTVDKSRWQQGNVFSCSVMHEALHNHYTQKSLS | |
| S354C, T366W) | LSPG | |
| Fc - IgGI | DKTHTCPPCPAPEAAGGPSVFLFPPKPKDTLMISRTPEVTCVVVSV | 166 |
| KEMA AAS- | SHEDPEVKFNWYVDGVEVHNAKTKPREEQYNSTYRVVSVLTVL | |
| HoleCC (L234A, | HQDWLNGKEYKCKVSNKALPAPIEKTISKAKGQPREPQVCTLPPS | |
| L235A, D265S; | REEMTKNQVSLSCAVKGFYPSDIAVEWESNGQPENNYKTTPPVL | |
| D356E, L358M; | DSDGSFFLVSKLTVDKSRWQQGNVFSCSVMHEALHNHYTQKSLS | |
| Y349C, T366S, | LSPG | |
| L368A, Y407V) | | |
| Fc- IgG2.3 | ERKSCVECPPCPAPPVAGKSVFLFPPKPKDTLMISRTPEVTCVVVD | 167 |
| P238K-Knob | VSHEDPEVQFNWYVDGVEVHNAKTKPREEQFNSTFRVVSVLTVV | |
| (P238K; | HQDWLNGKEYKCKVSNKGLPAPIEKTISKTKGQPREPQVYTLPPC | |
| D356E, L358M; | REEMTKNQVSLWCLVKGFYPSDIAVEWESNGQPENNYKTTPPML | |
| S354C, T366W) | DSDGSFFLYSKLTVDKSRWQQGNVFSCSVMHEALHNHYTQKSLS | |
| | LSPG | |
| Fc - IgG2.3 | ERKSCVECPPCPAPPVAGKSVFLFPPKPKDTLMISRTPEVTCVVVD | 168 |
| P238K-Hole | VSHEDPEVQFNWYVDGVEVHNAKTKPREEQFNSTFRVVSVLTVV | |
| (P238K; | HQDWLNGKEYKCKVSNKGLPAPIEKTISKTKGQPREPQVCTLPPS | |
| D356E, L358M; | REEMTKNQVSLSCAVKGFYPSDIAVEWESNGQPENNYKTTPPML | |
| Y349C, T366S, | DSDGSFFLVSKLTVDKSRWQQGNVFSCSVMHEALHNHYTQKSLS | |
| L368A, Y407V) | LSPG | |
| IgG1 CH1 | ASTKGPSVFPLAPSSKSTSGGTAALGCLVKDYFPEPVTVSWNSGA | 169 |
| | LTSGVHTFPAVLQSSGLYSLSSVVTVPSSSLGTQTYICNVNHKPSN | |
| | TKVDKKVEPKSC | |
| IgG2 CH1 | ASTKGPSVFPLAPCSRSTSESTAALGCLVKDYFPEPVTVSWNSGA | 170 |
| | LTSGVHTFPAVLQSSGLYSLSSVVTVPSSNFGTQTYTCNVDHKPS | |
| | NTKVDKTV | |
| Constant | RTVAAPSVFIFPPSDEQLKSGTASVVCLLNNFYPREAKVQWKVDN | 171 |
| Kappa | ALQSGNSQESVTEQDSKDSTYSLSSTLTLSKADYEKHKVYACEVT | |
| | HQGLSSPVTKSFNRGEC | |

TABLE E9
Full TCEs

| | | SEQ ID |
|---|---|---|
| Description | Amino acid Sequence | NO: |
| TCE001 | QVQLQQWGAGLLKPSETLSLTCAVYGGSFSDYYWSWVRQPPGK | 172 |
| Chain 1 | CLEWIGEITHSGSTNYNPSLKSRVTISVDTSKNQFSLKLSSVTAAD | |
| Heavy chain | TAVYYCARGGLDIFTGPYYFDSWGQGTLVTVSSGGGGSGGGGSG | |
| amino acid | GGGSDIQMTQSPSSLSASVGDRVTITCRASQEIRKDLAWYQQKPG | |
| sequence | KAPKLLIYGASSLRSGVPSRFSGSGSGTDFTLTISSLQPEDFATYYC | |
| | QQDYNYPPTFGCGTKVEIKDKTHTCPPCPAPEAAGGPSVFLFPPKP | |
| | KDTLMISRTPEVTCVVVSVSHEDPEVKFNWYVDGVEVHNAKTKP | |
| | REEQYNSTYRVVSVLTVLHQDWLNGKEYKCKVSNKALPAPIEKT | |
| | ISKAKGQPREPQVCTLPPSREEMTKNQVSLSCAVKGFYPSDIAVE | |
| | WESNGQPENNYKTTPPVLDSDGSFFLVSKLTVDKSRWQQGNVFS | |
| | CSVMHEALHNHYTQKSLSLSPG | |
| TCE001 | QVQLVESGGGVVQPGGSLRLSCAASGFTFSRYGIHWVRQAPGKC | 173 |
| Heavy chain | LEWVAAIWYDASKTYYADSVKGRFTISRDNSKNTLYLQMNSLRA | |
| Chain 2 | EDTAVYYCARSNYDILTRYYQYGLDVWGQGTTVTVSSGGGGSG | |
| amino acid | GGGSGGGGSAIQLTQSPSSLSASVGDRVTITCRASQGISSALAWY | |
| sequence | QQKPGKAPKLLIYDASSLESGVPSRFSGSGSGTDFTLTISSLQPEDF | |
| | ATYYCQQFKSYPRTFGCGTKVEIKDKTHTCPPCPAPEAAGGPSVFLF | |
| | PPKPKDTLMISRTPEVTCVVVSVSHEDPEVKFNWYVDGVEVHNAKTKP | |
| | REEQYNSTYRVVSVLTVLHQDWLNGKEYKCKVSNKALPAPIEKTISKA | |
| | KGQPREPQVYTLPPCREEMTKNQVSLWCLVKGFYPSDIAVEWESNGQP | |
| | ENNYKTTPPVLDSDGSFFLYSKLTVDKSRWQQGNVFSCSVMHEALHNH | |
| | YTQKSLSLSPG | |
| TCE002 | QVQLQQWGAGLLKPSETLSLTCAVYGGSFSDYYWSWVRQPPGK | 174 |
| Chain 1 | CLEWIGEITHSGSTNYNPSLKSRVTISVDTSKNQFSLKLSSVTAAD | |
| Heavy chain | TAVYYCARGGLDIFTGPYYFDSWGQGTLVTVSSGGGGSGGGGSG | |
| amino acid | GGGSDIQMTQSPSSLSASVGDRVTITCRASQEIRKDLAWYQQKPG | |
| sequence | KAPKLLIYGASSLRSGVPSRFSGSGSGTDFTLTISSLQPEDFATYYC | |
| | QQDYNYPPTFGCGTKVEIKDKTHTCPPCPAPEAAGGPSVFLFPPKP | |
| | KDTLMISRTPEVTCVVVSVSHEDPEVKFNWYVDGVEVHNAKTKP | |
| | REEQYNSTYRVVSVLTVLHQDWLNGKEYKCKVSNKALPAPIEKT | |
| | ISKAKGQPREPQVCTLPPSREEMTKNQVSLSCAVKGFYPSDIAVE | |
| | WESNGQPENNYKTTPPVLDSDGSFFLVSKLTVDKSRWQQGNVFS | |
| | CSVMHEALHNHYTQKSLSLSPG | |
| TCE002 | QVQLVESGGGVVQPGGSLRLSCAASGFTFSRYGIHWVRQAPGKG | 175 |
| Heavy chain | LEWVAAIWYDASKTYYADSVKGRFTISRDNSKNTLYLQMNSLRA | |
| Chain 2 | EDTAVYYCARSNYDILTRYYQYGLDVWGQGTTVTVSSASTKGPS | |
| amino acid | VFPLAPSSKSTSGGTAALGCLVKDYFPEPVTVSWNSGALTSGVHT | |
| sequence | FPAVLQSSGLYSLSSVVTVPSSSLGTQTYICNVNHKPSNTKVDKK | |
| | VEPKSCDKTHTCPPCPAPEAAGGPSVFLFPPKPKDTLMISRTPEVT | |
| | CVVVSVSHEDPEVKFNWYVDGVEVHNAKTKPREEQYNSTYRVV | |
| | SVLTVLHQDWLNGKEYKCKVSNKALPAPIEKTISKAKGQPREPQ | |
| | VYTLPPCREEMTKNQVSLWCLVKGFYPSDIAVEWESNGQPENNY | |
| | KTTPPVLDSDGSFFLYSKLTVDKSRWQQGNVFSCSVMHEALHNH | |
| | YTQKSLSLSPG | |
| TCE002 | AIQLTQSPSSLSASVGDRVTITCRASQGISSALAWYQQKPGKAPKL | 176 |
| Light chain | LIYDASSLESGVPSRFSGSGSGTDFTLTISSLQPEDFATYYCQQFKS | |
| Chain 3 | YPRTFGQGTKVEIKRTVAAPSVFIFPPSDEQLKSGTASVVCLLNNF | |
| amino acid | YPREAKVQWKVDNALQSGNSQESVTEQDSKDSTYSLSSTLTLSK | |
| sequence | ADYEKHKVYACEVTHQGLSSPVTKSFNRGEC | |
| TCE003 | QVQLQQWGAGLLKPSETLSLTCAVYGGSFSDYYWSWVRQPPGK | 177 |
| Chain 1 | CLEWIGEITHSGSTNYNPSLKSRVTISVDTSKNQFSLKLSSVTAAD | |
| Heavy chain | TAVYYCARGGLDIFTGPYYFDSWGQGTLVTVSSGGGGSGGGGSG | |
| amino acid | GGGSDIQMTQSPSSLSASVGDRVTITCRASQEIRKDLAWYQQKPG | |
| sequence | KAPKLLIYGASSLRSGVPSRFSGSGSGTDFTLTISSLQPEDFATYYC | |
| | QQDYNYPPTFGCGTKVEIKDKTHTCPPCPAPEAAGGPSVFLFPPKP | |
| | KDTLMISRTPEVTCVVVSVSHEDPEVKFNWYVDGVEVHNAKTKP | |
| | REEQYNSTYRVVSVLTVLHQDWLNGKEYKCKVSNKALPAPIEKT | |
| | ISKAKGQPREPQVCTLPPSREEMTKNQVSLSCAVKGFYPSDIAVE | |
| | WESNGQPENNYKTTPPVLDSDGSFFLVSKLTVDKSRWQQGNVFS | |
| | CSVMHEALHNHYTQKSLSLSPG | |
| TCE003 | QVQLVESGGGVVQPGGSLRLSCAASGFTFSRYGIHWVRQAPGKC | 178 |
| Heavy chain | LEWVAAIWYDASKTYYADSVKGRFTISRDNSKNTLYLQMNSLRA | |
| Chain 2 | EDTAVYYCARSNYDILTRYYSHGINVWGQGTTVTVSSGGGGSGG | |
| amino acid | GGSGGGGSAIQLTQSPSSLSASVGDRVTITCRASQGISSALAWYQ | |
| sequence | QKPGKAPKLLIYDASSLESGVPSRFSGSGSGTDFTLTISSLQPEDFA | |
| | TYYCQQFNSYPRTFGCGTKVEIKDKTHTCPPCPAPEAAGGPSVFL | |
| | FPPKPKDTLMISRTPEVTCVVVSVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWESNG | |
| | QPENNYKTTPPVLDSDGSFFLYSKLTVDKSRWQQGNVFSCSVMHEALH | |
| | NHYTQKSLSLSPG | |
| TCE004 | QVQLQQWGAGLLKPSETLSLTCAVYGGSFSDYYWSWVRQPPGK | 179 |
| Chain 1 | CLEWIGEITHSGSTNYNPSLKSRVTISVDTSKNQFSLKLSSVTAAD | |
| Heavy chain | TAVYYCARGGLDIFTGPYYFDSWGQGTLVTVSSGGGGSGGGGSG | |
| amino acid | GGGSDIQMTQSPSSLSASVGDRVTITCRASQEIRKDLAWYQQKPG | |
| sequence | KAPKLLIYGASSLRSGVPSRFSGSGSGTDFTLTISSLQPEDFATYYC | |
| | QQDYNYPPTFGCGTKVEIKDKTHTCPPCPAPEAAGGPSVFLFPPKP | |
| | KDTLMISRTPEVTCVVVSVSHEDPEVKFNWYVDGVEVHNAKTKP | |
| | REEQYNSTYRVVSVLTVLHQDWLNGKEYKCKVSNKALPAPIEKT | |
| | ISKAKGQPREPQVCTLPPSREEMTKNQVSLSCAVKGFYPSDIAVE | |
| | WESNGQPENNYKTTPPVLDSDGSFFLVSKLTVDKSRWQQGNVFS | |
| | CSVMHEALHNHYTQKSLSLSPG | |
| TCE004 | QVQLVESGGGVVQPGGSLRLSCAASGFTFSRYGIHWVRQAPGKG | 180 |
| Heavy chain | LEWVAAIWYDASKTYYADSVKGRFTISRDNSKNTLYLQMNSLRA | |
| Chain 2 | EDTAVYYCARSNYDILTRYYSHGINVWGQGTTVTVSSASTKGPSV | |
| amino acid | FPLAPSSKSTSGGTAALGCLVKDYFPEPVTVSWNSGALTSGVHTFPAVL | |
| sequence | QSSGLYSLSSVVTVPSSSLGTQTYICNVNHKPSNTKVDKKVEPKSCDKT | |
| | HTCPPCPAPEAAGGPSVFLFPPKPKDTLMISRTPEVTCVVVSVSHEDPEV | |
| | KFNWYVDGVEVHNAKTKPREEQYNSTYRVVSVLTVLHQDWLNGKEY | |
| | KCKVSNKALPAPIEKTISKAKGQPREPQVYTLPPCREEMTKNQVSLWCL | |
| | VKGFYPSDIAVEWESNGQPENNYKTTPPVLDSDGSFFLYSKLTVDKSRW | |
| | QQGNVFSCSVMHEALHNHYTQKSLSLSPG | |
| TCE004 | AIQLTQSPSSLSASVGDRVTITCRASQGISSALAWYQQKPGKAPKL | 181 |
| Light chain | LIYDASSLESGVPSRFSGSGSGTDFTLTISSLQPEDFATYYCQQFNS | |
| Chain 3 | YPRTFGQGTKVEIKRTVAAPSVFIFPPSDEQLKSGTASVVCLLNNF | |
| amino acid | YPREAKVQWKVDNALQSGNSQESVTEQDSKDSTYSLSSTLTLSK | |
| sequence | ADYEKHKVYACEVTHQGLSSPVTKSFNRGEC | |
| TCE005 | EVQLVQSGAEVKKPGESLKISCKGSGYSFTSYWIGWVRQMPGKC | 182 |
| Chain 1 | LEWMGIIYPGDSDTRYSPSFQGQVTISADKSISTAYLQWSSLKASD | |
| Heavy chain | TAMYYCARGGTTIFGVVFEYWGQGTLVTVSSGGGGSGGGGSGG | |
| amino acid | GGSDIQMTQSPSSLSASVGDRVTITCRASQSINNYLNWYQQKPGK | |
| sequence | APKLLIYGASSLQSGVPSRFSGSGSGTDFTLTISSLQPEDFATYYCQ | |
| | QTYSTPWTFGCGTKVEIKDKTHTCPPCPAPEAAGGPSVFLFPPKPK | |
| | DTLMISRTPEVTCVVVSVSHEDPEVKFNWYVDGVEVHNAKTKPR | |
| | EEQYNSTYRVVSVLTVLHQDWLNGKEYKCKVSNKALPAPIEKTI | |
| | SKAKGQPREPQVCTLPPSREEMTKNQVSLSCAVKGFYPSDIAVEW | |
| | ESNGQPENNYKTTPPVLDSDGSFFLVSKLTVDKSRWQQGNVFSCS | |
| | VMHEALHNHYTQKSLSLSPG | |
| TCE005 | QVQLVESGGGVVQPGGSLRLSCAASGFTFSRYGIHWVRQAPGKC | 183 |
| Heavy chain | LEWVAAIWYDASKTYYADSVKGRFTISRDNSKNTLYLQMNSLRA | |
| Chain 2 | EDTAVYYCARSNYDILTRYYQYGLDVWGQGTTVTVSSGGGGSG | |
| amino acid | GGGSGGGGSAIQLTQSPSSLSASVGDRVTITCRASQGISSALAWY | |
| sequence | QQKPGKAPKLLIYDASSLESGVPSRFSGSGSGTDFTLTISSLQPEDF | |
| | ATYYCQQFKSYPRTFGCGTKVEIKDKTHTCPPCPAPEAAGGPSVFLF | |
| | PPKPKDTLMISRTPEVTCVVVSVSHEDPEVKFNWYVDGVEVHNAKTKP | |
| | REEQYNSTYRVVSVLTVLHQDWLNGKEYKCKVSNKALPAPIEKTISKA | |
| | KGQPREPQVYTLPPCREEMTKNQVSLWCLVKGFYPSDIAVEWESNGQP | |
| | ENNYKTTPPVLDSDGSFFLYSKLTVDKSRWQQGNVFSCSVMHEALHNH | |
| | YTQKSLSLSPG | |
| TCE006 | EVQLVQSGAEVKKPGESLKISCKGSGYSFTSYWIGWVRQMPGKC | 184 |
| Chain 1 | LEWMGIIYPGDSDTRYSPSFQGQVTISADKSISTAYLQWSSLKASD | |
| Heavy chain | TAMYYCARGGTTIFGVVFEYWGQGTLVTVSSGGGGSGGGGSGG | |
| amino acid | GGSDIQMTQSPSSLSASVGDRVTITCRASQSINNYLNWYQQKPGK | |
| sequence | APKLLIYGASSLQSGVPSRFSGSGSGTDFTLTISSLQPEDFATYYCQ | |
| | QTYSTPWTFGCGTKVEIKDKTHTCPPCPAPEAAGGPSVFLFPPKPK | |
| | DTLMISRTPEVTCVVVSVSHEDPEVKFNWYVDGVEVHNAKTKPR | |
| | EEQYNSTYRVVSVLTVLHQDWLNGKEYKCKVSNKALPAPIEKTI | |
| | SKAKGQPREPQVCTLPPSREEMTKNQVSLSCAVKGFYPSDIAVEW | |
| | ESNGQPENNYKTTPPVLDSDGSFFLVSKLTVDKSRWQQGNVFSCS | |
| | VMHEALHNHYTQKSLSLSPG | |
| TCE006 | QVQLVESGGGVVQPGGSLRLSCAASGFTFSRYGIHWVRQAPGKG | 185 |
| Heavy chain | LEWVAAIWYDASKTYYADSVKGRFTISRDNSKNTLYLQMNSLRA | |
| Chain 2 | EDTAVYYCARSNYDILTRYYQYGLDVWGQGTTVTVSSASTKGPS | |
| amino acid | VFPLAPSSKSTSGGTAALGCLVKDYFPEPVTVSWNSGALTSGVHTFPAV | |
| sequence | LQSSGLYSLSSVVTVPSSSLGTQTYICNVNHKPSNTKVDKKVEPKSCDK | |
| | THTCPPCPAPEAAGGPSVFLFPPKPKDTLMISRTPEVTCVVVSVSHEDPE | |
| | VKFNWYVDGVEVHNAKTKPREEQYNSTYRVVSVLTVLHQDWLNGKE | |
| | YKCKVSNKALPAPIEKTISKAKGQPREPQVYTLPPCREEMTKNQVSLWC | |
| | LVKGFYPSDIAVEWESNGQPENNYKTTPPVLDSDGSFFLYSKLTVDKSR | |
| | WQQGNVFSCSVMHEALHNHYTQKSLSLSPG | |
| TCE006 | AIQLTQSPSSLSASVGDRVTITCRASQGISSALAWYQQKPGKAPKL | 186 |
| Light chain | LIYDASSLESGVPSRFSGSGSGTDFTLTISSLQPEDFATYYCQQFKS | |
| Chain 3 | YPRTFGQGTKVEIKRTVAAPSVFIFPPSDEQLKSGTASVVCLLNNF | |
| amino acid | YPREAKVQWKVDNALQSGNSQESVTEQDSKDSTYSLSSTLTLSK | |
| sequence | ADYEKHKVYACEVTHQGLSSPVTKSFNRGEC | |
| TCE007 | EVQLVQSGAEVKKPGESLKISCKGSGYSFTSYWIGWVRQMPGKC | 187 |
| Chain 1 | LEWMGIIYPGDSDTRYSPSFQGQVTISADKSISTAYLQWSSLKASD | |
| Heavy chain | TAMYYCARGGTTIFGVVFEYWGQGTLVTVSSGGGGSGGGGSGG | |
| amino acid | GGSDIQMTQSPSSLSASVGDRVTITCRASQSINNYLNWYQQKPGK | |
| sequence | APKLLIYGASSLQSGVPSRFSGSGSGTDFTLTISSLQPEDFATYYCQ | |
| | QTYSTPWTFGCGTKVEIKDKTHTCPPCPAPEAAGGPSVFLFPPKPK | |
| | DTLMISRTPEVTCVVVSVSHEDPEVKFNWYVDGVEVHNAKTKPR | |
| | EEQYNSTYRVVSVLTVLHQDWLNGKEYKCKVSNKALPAPIEKTI | |
| | SKAKGQPREPQVCTLPPSREEMTKNQVSLSCAVKGFYPSDIAVEW | |
| | ESNGQPENNYKTTPPVLDSDGSFFLVSKLTVDKSRWQQGNVFSCS | |
| | VMHEALHNHYTQKSLSLSPG | |
| TCE007 | QVQLVESGGGVVQPGGSLRLSCAASGFTFSRYGIHWVRQAPGKC | 188 |
| Heavy chain | LEWVAAIWYDASKTYYADSVKGRFTISRDNSKNTLYLQMNSLRA | |
| Chain 2 | EDTAVYYCARSNYDILTRYYSHGINVWGQGTTVTVSSGGGGSGG | |
| amino acid | GGSGGGGSAIQLTQSPSSLSASVGDRVTITCRASQGISSALAWYQ | |
| sequence | QKPGKAPKLLIYDASSLESGVPSRFSGSGSGTDFTLTISSLQPEDFA | |
| | TYYCQQFNSYPRTFGCGTKVEIKDKTHTCPPCPAPEAAGGPSVFL | |
| | FPPKPKDTLMISRTPEVTCVVVSVSHEDPEVKFNWYVDGVEVHNAK | |
| | TKPREEQYNSTYRVVSVLTVLHQDWLNGKEYKCKVSNKALPAPIEKTIS | |
| | KAKGQPREPQVYTLPPCREEMTKNQVSLWCLVKGFYPSDIAVEWESNG | |
| | QPENNYKTTPPVLDSDGSFFLYSKLTVDKSRWQQGNVFSCSVMHEALH | |
| | NHYTQKSLSLSPG | |
| TCE008 | EVQLVQSGAEVKKPGESLKISCKGSGYSFTSYWIGWVRQMPGKC | 189 |
| Chain 1 | LEWMGIIYPGDSDTRYSPSFQGQVTISADKSISTAYLQWSSLKASD | |
| Heavy chain | TAMYYCARGGTTIFGVVFEYWGQGTLVTVSSGGGGSGGGGSGG | |
| amino acid | GGSDIQMTQSPSSLSASVGDRVTITCRASQSINNYLNWYQQKPGK | |
| sequence | APKLLIYGASSLQSGVPSRFSGSGSGTDFTLTISSLQPEDFATYYCQ | |
| | QTYSTPWTFGCGTKVEIKDKTHTCPPCPAPEAAGGPSVFLFPPKPK | |
| | DTLMISRTPEVTCVVVSVSHEDPEVKFNWYVDGVEVHNAKTKPR | |
| | EEQYNSTYRVVSVLTVLHQDWLNGKEYKCKVSNKALPAPIEKTI | |
| | SKAKGQPREPQVCTLPPSREEMTKNQVSLSCAVKGFYPSDIAVEW | |
| | ESNGQPENNYKTTPPVLDSDGSFFLVSKLTVDKSRWQQGNVFSCS | |
| | VMHEALHNHYTQKSLSLSPG | |
| TCE008 | QVQLVESGGGVVQPGGSLRLSCAASGFTFSRYGIHWVRQAPGKG | 190 |
| Heavy chain | LEWVAAIWYDASKTYYADSVKGRFTISRDNSKNTLYLQMNSLRA | |
| Chain 2 | EDTAVYYCARSNYDILTRYYSHGINVWGQGTTVTVSSASTKGPSV | |
| amino acid | FPLAPSSKSTSGGTAALGCLVKDYFPEPVTVSWNSGALTSGVHTFPAVL | |
| sequence | QSSGLYSLSSVVTVPSSSLGTQTYICNVNHKPSNTKVDKKVEPKSCDKT | |
| | HTCPPCPAPEAAGGPSVFLFPPKPKDTLMISRTPEVTCVVVSVSHEDPEV | |
| | KFNWYVDGVEVHNAKTKPREEQYNSTYRVVSVLTVLHQDWLNGKEY | |
| | KCKVSNKALPAPIEKTISKAKGQPREPQVYTLPPCREEMTKNQVSLWCL | |
| | VKGFYPSDIAVEWESNGQPENNYKTTPPVLDSDGSFFLYSKLTVDKSRW | |
| | QQGNVFSCSVMHEALHNHYTQKSLSLSPG | |
| TCE008 | AIQLTQSPSSLSASVGDRVTITCRASQGISSALAWYQQKPGKAPKL | 191 |
| Light chain | LIYDASSLESGVPSRFSGSGSGTDFTLTISSLQPEDFATYYCQQFNS | |
| Chain 3 | YPRTFGQGTKVEIKRTVAAPSVFIFPPSDEQLKSGTASVVCLLNNF | |
| amino acid | YPREAKVQWKVDNALQSGNSQESVTEQDSKDSTYSLSSTLTLSK | |
| sequence | ADYEKHKVYACEVTHQGLSSPVTKSFNRGEC | |
| TCE009 | QVQLQQWGAGLLKPSETLSLTCAVYGGSFSDYYWSWVRQPPGK | 234 |
| Chain 1 | CLEWIGEITHSGSTNYNPSLKSRVTISVDTSKNQFSLKLSSVTAAD | |
| Heavy chain | TAVYYCARGGLDIFTGPYYFDSWGQGTLVTVSSGGGGSGGGGSG | |
| amino acid | GGGSDIQMTQSPSSLSASVGDRVTITCRASQEIRKDLAWYQQKPG | |
| sequence | KAPKLLIYGASSLRSGVPSRFSGSGSGTDFTLTISSLQPEDFATYYC | |
| | QQDYNYPPTFGCGTKVEIKERKSCVECPPCPAPPVAGKSVFLFPPK | |
| | PKDTLMISRTPEVTCVVVDVSHEDPEVQFNWYVDGVEVHNAKT | |
| | KPREEQFNSTFRVVSVLTVVHQDWLNGKEYKCKVSNKGLPAPIE | |
| | KTISKTKGQPREPQVCTLPPSREEMTKNQVSLSCAVKGFYPSDIAV | |
| | EWESNGQPENNYKTTPPMLDSDGSFFLVSKLTVDKSRWQQGNVF | |
| | SCSVMHEALHNHYTQKSLSLSPG | |
| TCE009 | QVQLVESGGGVVQPGGSLRLSCAASGFTFSRYGIHWVRQAPGKG | 235 |
| Heavy chain | LEWVAAIWYDASKTYYADSVKGRFTISRDNSKNTLYLQMNSLRA | |
| Chain 2 | EDTAVYYCARSNYDILTRYYQYGLDVWGQGTTVTVSSASTKGPS | |
| amino acid | VFPLAPCSRSTSESTAALGCLVKDYFPEPVTVSWNSGALTSGVHT | |
| sequence | FPAVLQSSGLYSLSSVVTVPSSNFGTQTYTCNVDHKPSNTKVDKT | |
| | VERKSCVECPPCPAPPVAGKSVFLFPPKPKDTLMISRTPEVTCVVV | |
| | DVSHEDPEVQFNWYVDGVEVHNAKTKPREEQFNSTFRVVSVLTV | |
| | VHQDWLNGKEYKCKVSNKGLPAPIEKTISKTKGQPREPQVYTLPP | |
| | CREEMTKNQVSLWCLVKGFYPSDIAVEWESNGQPENNYKTTPPM | |
| | LDSDGSFFLYSKLTVDKSRWQQGNVFSCSVMHEALHNHYTQKSL | |
| | SLSPG | |
| TCE009 | AIQLTQSPSSLSASVGDRVTITCRASQGISSALAWYQQKPGKAPKL | 236 |
| Light chain | LIYDASSLESGVPSRFSGSGSGTDFTLTISSLQPEDFATYYCQQFKS | |
| Chain 3 | YPRTFGQGTKVEIKRTVAAPSVFIFPPSDEQLKSGTASVVCLLNNF | |
| amino acid | YPREAKVQWKVDNALQSGNSQESVTEQDSKDSTYSLSSTLTLSK | |
| sequence | ADYEKHKVYACEVTHQGLSSPVTKSFNRGEC | |
| TCE010 | QVQLQQWGAGLLKPSETLSLTCAVYGGSFSDYYWSWVRQPPGK | 237 |
| Chain 1 | CLEWIGEITHSGSTNYNPSLKSRVTISVDTSKNQFSLKLSSVTAAD | |
| Heavy chain | TAVYYCARGGLDIFTGPYYFDSWGQGTLVTVSSGGGGSGGGGSG | |
| amino acid | GGGSDIQMTQSPSSLSASVGDRVTITCRASQEIRKDLAWYQQKPG | |
| sequence | KAPKLLIYGASSLRSGVPSRFSGSGSGTDFTLTISSLQPEDFATYYC | |
| | QQDYNYPPTFGCGTKVEIKERKSCVECPPCPAPPVAGKSVFLFPPK | |
| | PKDTLMISRTPEVTCVVVDVSHEDPEVQFNWYVDGVEVHNAKT | |
| | KPREEQFNSTFRVVSVLTVVHQDWLNGKEYKCKVSNKGLPAPIE | |
| | KTISKTKGQPREPQVCTLPPSREEMTKNQVSLSCAVKGFYPSDIAV | |
| | EWESNGQPENNYKTTPPMLDSDGSFFLVSKLTVDKSRWQQGNVF | |
| | SCSVMHEALHNHYTQKSLSLSPG | |
| TCE010 | QVQLVESGGGVVQPGGSLRLSCAASGFTFSRYGIHWVRQAPGKG | 238 |
| Heavy chain | LEWVAAIWYDASKTYYADSVKGRFTISRDNSKNTLYLQMNSLRA | |
| Chain 2 | EDTAVYYCARSNYDILTRYYSHGINVWGQGTTVTVSSASTKGPS | |
| amino acid | VFPLAPCSRSTSESTAALGCLVKDYFPEPVTVSWNSGALTSGVHT | |
| sequence | FPAVLQSSGLYSLSSVVTVPSSNFGTQTYTCNVDHKPSNTKVDKT | |
| | VERKSCVECPPCPAPPVAGKSVFLFPPKPKDTLMISRTPEVTCVVV | |
| | DVSHEDPEVQFNWYVDGVEVHNAKTKPREEQFNSTFRVVSVLTV | |
| | VHQDWLNGKEYKCKVSNKGLPAPIEKTISKTKGQPREPQVYTLPP | |
| | CREEMTKNQVSLWCLVKGFYPSDIAVEWESNGQPENNYKTTPPM | |
| | LDSDGSFFLYSKLTVDKSRWQQGNVFSCSVMHEALHNHYTQKSL | |
| | SLSPG | |
| TCE010 | AIQLTQSPSSLSASVGDRVTITCRASQGISSALAWYQQKPGKAPKL | 239 |
| Light chain | LIYDASSLESGVPSRFSGSGSGTDFTLTISSLQPEDFATYYCQQFNS | |
| Chain 3 | YPRTFGQGTKVEIKRTVAAPSVFIFPPSDEQLKSGTASVVCLLNNF | |
| amino acid | YPREAKVQWKVDNALQSGNSQESVTEQDSKDSTYSLSSTLTLSK | |
| sequence | ADYEKHKVYACEVTHQGLSSPVTKSFNRGEC | |

Note:
Kabat VH and VL sequences in the TCEs are underlined

### Example 10: High Throughput Screening for TCE-Mediated Depletion of Raji Cells

This Example utilized high throughput screening to identify TCEs capable of depleting Raji cells, an immortalized B-cell lymphoma line that expresses surface CD19 and serves as a model for B cells. Multiple 0.1 μM (10× the top concentration to be screened) solutions of TCE were prepared by diluting TCE in cell culture media containing Roswell Park Memorial Institute (RPMI) medium (Gibco; Catalog No. 11875093), 10% HI FBS (Gibco; Catalog No. 10062-147), and 1% Penicillin/streptomycin (Gibco; Catalog No. 15140122). A dose titration of TCE was obtained by diluting TCE solutions 1:10 in cell culture media to yield 10× working solutions of 100 nM, 10 nM, 1 nM, 0.1 nM, 0.01 nM, and 0.001 nM.

Solutions of eGFP/Firefly Luciferase Raji cells (BPS Bioscience; Catalog No. 78916) were resuspended to approximately 1×10⁶ cells/mL in cell culture media. Cryopreserved human peripheral blood Pan T cells (StemCell; Catalog No. 70024) were thawed and resuspended to a concentration of approximately 0.5×10⁶ cells/mL in cell culture media.

A 20 μL volume of the 10× working solutions of diluted TCE was added per well of a 96 well black, flat-bottom plate (Corning; Catalog No. 3603). The prepared Raji and T cells were added at a ratio of 4 T cells: 1 Raji cell by adding 20 μL (approximately 20,000) Raji cells and 160 μL (approximately 80,000) T cells per well. Addition of cell suspensions resulted in a 1:10 dilution of TCE to yield final concentrations of 10 nM, 1 nM, 0.1 nM, 0.01 nM, 0.001 nM, and 0.0001 nM TCE per well.

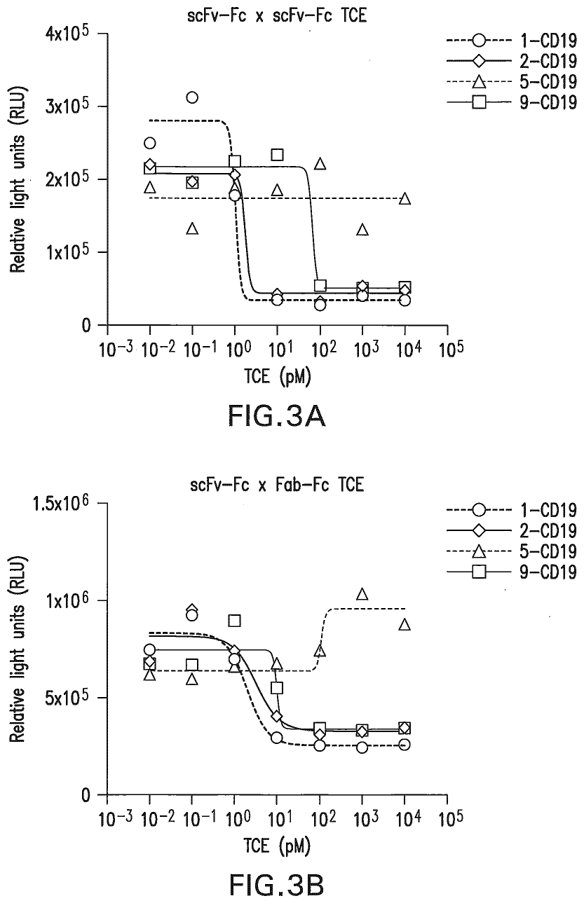

Culture plates were incubated at 37° C., 5% CO₂ for approximately 72 hours after which time plates were centrifuged to pellet cells. A 100 μL volume of supernatant was decanted and 100 μL of luciferase substrate (Promega; Catalog No. E6120) was added per well. Luciferase activity was quantified on a GloMax Plate Reader (integration time: 0.2 s without lid). Multiple CD19 binders were screened paired with the anti-CD3 “3-CD3” binder in two TCE formats, i.e., the scFv-Fc×Fab-Fc or scFv-Fc×scFv-Fc formats shown in FIG. 2A and FIG. 2B, respectively. Luciferase activity was assessed as a surrogate for cell number and was reported as relative light units (RLU) in FIG. 3A (i.e., using the scFv-Fc×scFv-Fc TCE format) and FIG. 3B (i.e., using the scFv-Fc×Fab-Fc TCE format). Potency was reported as IC₅₀, defined as the concentration that reduces luciferase signal by 50%, as estimated by nonlinear regression of concentration-response data using a four-parameter logistic model in GraphPad Prism (version 10.4.0) (Table E10 and Table E11). Percentage depletion of Raji cells was calculated by normalizing the RLU in treated samples to untreated control samples. The maximum observed depletion (Max depletion %) in response to each TCE was calculated and reported in Table E10 and Table E11. These results demonstrated that the CD19 binders “1-CD19” and “2-CD19” exhibited enhanced depletion compared to certain other CD19 binders when paired with the anti-CD3 binder “3-CD3” in either an scFv Fc×Fab or scFv Fc×scFv Fc bispecific TCE format (FIG. 3A and FIG. 3B).

The effect of hinge geometry on depletion was then evaluated through incorporation of either GGGGS(G4S; SEQ ID NO: 160), GGGGSGGGGS ((G4S)2; SEQ ID NO: 161), or GGGGSGGGGSGGGGS ((G4S)3; SEQ ID NO:162) linkers between the CD19 binder “1-CD19” scFv and the Fc domain and/or the CD3 binder “3-CD3” scFv and the Fc domain. No significant effect of linker length on depletion activity was observed based on the linkers tested (Table E10 and Table E11, although additional testing of the CD19 scFvs and CD3 scFvs would be performed to further develop TCEs with desired characteristics (see Examples below)).

TABLE E10
Raji Depletion Maximum and IC₅₀ with TCEs
comprising varying CD19 binders^{1, 2}

| CD19 | CD3 | | Max | IC₅₀ |
|---|---|---|---|---|
| binder | binder | TCE format | Depletion % | (pM) |
| 3-CD19 | 3-CD3 | scFv-Fc x scFv-Fc | 83 | 4.2 |
| 4-CD19 | 3-CD3 | scFv-Fc x scFv-Fc | 73 | 10.7 |
| 5-CD19 | 3-CD3 | scFv-Fc x scFv-Fc | NA | NA |
| 6-CD19 | 3-CD3 | scFv-Fc x scFv-Fc | NA | NA |
| 2-CD19 | 3-CD3 | scFv-Fc x scFv-Fc | 82 | 1.8 |
| 7-CD19 | 3-CD3 | scFv-Fc x scFv-Fc | NA | NA |
| 8-CD19 | 3-CD3 | scFv-Fc x scFv-Fc | 67 | 167.1 |
| 9-CD19 | 3-CD3 | scFv-Fc x scFv-Fc | 70 | 66.4 |
| 10-CD19 | 3-CD3 | scFv-Fc x scFv-Fc | NA | NA |
| 1-CD19 | 3-CD3 | scFv-Fc x scFv-Fc | 84 | 1.0 |
| 3-CD19 | 3-CD3 | Fab-Fc x scFv-Fc | 56 | 8.6 |
| 4-CD19 | 3-CD3 | Fab-Fc x scFv-Fc | NA | NA |
| 5-CD19 | 3-CD3 | Fab-Fc x scFv-Fc | NA | NA |
| 6-CD19 | 3-CD3 | Fab-Fc x scFv-Fc | 41 | 92.5 |
| 2-CD19 | 3-CD3 | Fab-Fc x scFv-Fc | 49 | 3.3 |
| 7-CD19 | 3-CD3 | Fab-Fc x scFv-Fc | NA | NA |
| 8-CD19 | 3-CD3 | Fab-Fc x scFv-Fc | 37 | ND |
| 9-CD19 | 3-CD3 | Fab-Fc x scFv-Fc | 43 | 10.1 |
| 10-CD19 | 3-CD3 | Fab-Fc x scFv-Fc | NA | NA |
| 1-CD19 | 3-CD3 | Fab-Fc x scFv-Fc | 58 | 2.0 |

¹NA: No depletion observed within tested concentration range; IC₅₀ and Max depletion not calculated
²ND: not calculated due to unstable curve fit

TABLE E11
Effect of TCE hinge geometry on Raji B cell depletion³

| | | | Max | |
|---|---|---|---|---|
| CD19 | CD3 | | Depletion | IC₅₀ |
| binder | binder | TCE format | % | (pM) |
| 1-CD19 | 3-CD3 | scFv CD3-Fc x scFv CD19-Fc | 79 | 9.3 |
| 1-CD19 | 3-CD3 | scFv CD3-Fc x scFv CD19-(G4S)-Fc | 81 | 8.5 |
| 1-CD19 | 3-CD3 | scFv CD3-Fc x scFv CD19-(G4S)2-Fc | 78 | 7.3 |
| 1-CD19 | 3-CD3 | scFv CD3-Fc x scFv CD 19-(G4S)3-Fc | 81 | 6.8 |
| 1-CD19 | 3-CD3 | scFv CD3-(G4S)-Fc x scFv CD19-Fc | 89 | ND |
| 1-CD19 | 3-CD3 | scFv CD3-(G4S)-Fc x scFv CD19-(G4S)-Fc | 86 | 7.5 |
| 1-CD19 | 3-CD3 | scFv CD3-(G4S)-Fc x scFv CD19-(G4S)2-Fc | 85 | 8.4 |
| 1-CD19 | 3-CD3 | scFv CD3-(G4S)-Fc x scFv CD19-(G4S)3-Fc | 81 | 64.8 |
| 1-CD19 | 3-CD3 | scFv CD3-(G4S)2-Fc x scFv CD19-Fc | 81 | 10.3 |
| 1-CD19 | 3-CD3 | scFv CD3-(G4S)2-Fc x scFv CD19-(G4S)-Fc | 81 | 8.6 |
| 1-CD19 | 3-CD3 | scFv CD3-(G4S)2-Fc x scFv CD19-(G4S)2- | 72 | 8.4 |
| | | Fc | | |
| 1-CD19 | 3-CD3 | scFv CD3-(G4S)2-Fc x scFv CD19-(G4S)3- | 85 | 7.2 |
| | | Fc | | |
| 1-CD19 | 3-CD3 | scFv CD3-(G4S)3-Fc x scFv CD19-Fc | 87 | 8.0 |
| 1-CD19 | 3-CD3 | scFv CD3-(G4S)3-Fc x scFv CD19-(G4S)-Fc | 80 | 7.8 |
| 1-CD19 | 3-CD3 | scFv CD3-(G4S)3-Fc x scFv CD19-(G4S)2- | 82.41 | 0.8824 |
| | | Fc | | |
| 1-CD19 | 3-CD3 | scFv CD3-(G4S)3-Fc x scFv CD19-(G4S)3- | 81.77 | 7.571 |
| | | Fc | | |

³ND: not calculated due to unstable curve fit

The results from these studies indicated that 1-CD19 and 2-CD19 demonstrated enhanced activity in the depletion screening assay when compared to certain other CD19 binders screened.

### Example 11: Evaluation of the Impact of CD3 Binding Affinity on B Cell Depletion and Cytokine Release

The inventors hypothesized that a reduction in CD3 binding activity could enable a decoupling of cytokine release from the target cell depletion activity of a CD3×CD 19 bispecific antibody. To test this hypothesis, the inventors analyzed the ability of various TCEs to reduce cytokine induction through reduction in CD3 binding affinity. To evaluate the impact of CD3 binding affinity on the activity of CD19×CD3 TCEs, TCEs in the scFv-Fc×scFv-Fc format (i.e., as generally depicted in FIG. 1B) were generated comprising the 1-CD19 binder paired with several different CD3 binders of varying CD3 binding affinity (i.e., four (4) CD3 binders with binding affinities to CD3 ranging from “high” affinity to “low” affinity: (i) a “high” affinity CD3 binder; (ii) a “medium-high” affinity CD3 binder; (iii) a “medium-low” affinity CD3 binder; and (iv) a “low” affinity CD3 binder). The activity of the aforementioned TCEs to mediate depletion of primary human B cells and to induce cytokine release was subsequently evaluated in an in vitro human PBMC assay as briefly described below.

Cryopreserved human PBMCs from healthy donors were cultured with a 9-point, four-fold TCE dose titration for 72 hours. After incubation, cells were stained and analyzed by multicolor flow cytometry to quantify CD20+ B cell depletion versus controls. Supernatants were collected for cytokine measurement using the MSD U-plex4 assay, following the manufacturer's protocol.

Concentration-response curves for cytokine release and B cell depletion were fit using nonlinear regression with a four-parameter logistic model (GraphPad Prism software version 10.4.0). The results demonstrated that TCEs characterized with varying CD3 binding affinities maintained similar B cell depletion activity. In addition, and consistent with the inventors' hypothesis, these data showed a decrease in cytokine release levels with decreasing CD3 binding affinity of the TCEs tested (data not shown). Indeed, at comparable B cell depletion potency (EC₅₀), lower levels of cytokine secretion were observed in response to TCEs comprised of CD3 binders with reduced CD3 binding affinity (data not shown). These data demonstrated that modulation of the affinity of the CD3 binder can facilitate an enhanced therapeutic window that maximizes B cell depletion, while minimizing cytokine release activity.

### Example 12: Stabilization of CD19 and CD3 scFvs

This Example analyzed the extent that mutations to restore germline framework sequences and incorporate scFv disulfide bonds could be used to affect the stability of TCEs having CD19 scFvs and CD3 scFvs. As described above, 1-CD19 (see Table E10 and Table E11) and 2-CD19 (see Table E10) demonstrated enhanced activity in the depletion screening assay compared to certain other CD19 binders screened (Example 10). The variable domains of the CD19 binding sequence in 1-CD19 had mutations from germline in the framework regions of the sequence. The VH amino acid mutation A116T and V_{L} amino acid mutations E1D V3Q L4M V46L S61R were introduced to generate the binder “11-CD19.” The effects of disulfide introduction in the CD19 scFv were conducted (i) in the absence of these framework reversion mutants to generate the binder “12-CD19” as well as (ii) in the presence of these mutations to generate the binder “13-CD19.” Similarly, the 2-CD19 variable domain was modified to restore germline framework sequences by introducing V_{H} amino acid mutations Q1E L47W and V_{L} amino acid mutations E1D V3Q L4M T13A as well as introducing a disulfide bond to generate 14-CD19. Mutations to restore germline framework sequences in the CD3 binding sequences are described in Example 8.

The heavy chain variable regions (V_{H})s and light chain variable regions (V_{L})s along with the CDRs of the mutated anti-CD19 antibodies exemplified here (i.e., 11-CD19, 12-CD19, 13-CD19 and 14-CD19) are provided below.

Summary of CD19 Binding Domains Engineered from 1-CD19

### 11-CD19 Binder:

The heavy chain variable region (VH) amino acid sequence of 11-CD19 is described in SEQ ID NO: 60.

(SEQ ID NO: 60: CDR domains are in bold)
QVQLQQWGAGLLKPSETLSLTCAVYGGSFSDYYWSWVRQPPGKGLEWIG
EITHSGSTNYNPSLKSRVTISVDTSKNQFSLKLSSVTAADTAVYYCAR
GGLDIFTGPYYFDSWGQGTLVTVSS

The VH CDR amino acid sequences of 11-CD19 are as follows: DYYWS (VH CDR1; SEQ ID NO: 9); EITHSGSTNYNPSLKS (VH CDR2; SEQ ID NO: 10); GGLDIFTGPYYFDS (VH CDR3; SEQ ID NO: 11).

The light chain variable region (VL) amino acid sequence of 11-CD19 is described in SEQ ID NO: 61.

(SEQ ID NO: 61: CDR domains are in bold)
DIQMTQSPSSLSASVGDRVTITCRASQEIRKDLAWYQQKPGKAPKLLIY
GASSLRSGVPSRFSGSGSGTDFTLTISSLQPEDFATYYCQQDYNYPPTF
GQGTKVEIK

The VL CDR amino acid sequences of 11-CD19 are as follows: RASQEIRKDLA (VL CDR1; SEQ ID NO: 14); GASSLRS (VL CDR2; SEQ ID NO: 15); QQDYNYPPT (V_{L} CDR3; SEQ ID NO: 16).

### 12-CD19 Binder:

The heavy chain variable region (VH) amino acid sequence of 12-CD19 is described in SEQ ID NO: 62.

(SEQ ID NO: 62: CDR domains are in bold)
QVQLQQWGAGLLKPSETLSLTCAVYGGSFSDYYWSWVRQPPGKCLEWI
GEITHSGSTNYNPSLKSRVTISVDTSKNQFSLKLSSVTAADTAVYYCAR
GGLDIFTGPYYFDSWGQGALVTVSS

The VH CDR amino acid sequences of 12-CD19 are as follows: DYYWS (VH CDR1; SEQ ID NO: 9); EITHSGSTNYNPSLKS (VH CDR2; SEQ ID NO: 10); GGLDIFTGPYYFDS (VH CDR3; SEQ ID NO: 11).

The light chain variable region (VL) amino acid sequence of 12-CD19 is described in SEQ ID NO: 63.

(SEQ ID NO: 63: CDR domains are in bold)
EIVLTQSPSSLSASVGDRVTITCRASQEIRKDLAWYQQKPGKAPKVLIYG
ASSLRSGVPSSFSGSGSGTDFTLTISSLQPEDFATYYCQQDYNYPPTFGC
GTKVEIK

The VL CDR amino acid sequences of 12-CD19 are as follows: RASQEIRKDLA (VL CDR1; SEQ ID NO: 14); GASSLRS (VL CDR2; SEQ ID NO: 15); QQDYNYPPT (VL CDR3; SEQ ID NO: 16).

### 13-Cd19 Binder:

The heavy chain variable region (VH) amino acid sequence of 13-CD19 is described in SEQ ID NO: 12.

(SEQ ID NO: 12: CDR domains are in bold)
QVQLQQWGAGLLKPSETLSLTCAVYGGSFSDYYWSWVRQPPGKCLEWIGE
ITHSGSTNYNPSLKSRVTISVDTSKNQFSLKLSSVTAADTAVYYCARGGL
DIFTGPYYFDSWGQGTLVTVSS

The VH CDR amino acid sequences of 13-CD19 are as follows: DYYWS (VH CDR1; SEQ ID NO: 9); EITHSGSTNYNPSLKS (VH CDR2; SEQ ID NO: 10); GGLDIFTGPYYFDS (VH CDR3; SEQ ID NO: 11).

The light chain variable region (V_{L}) amino acid sequence of 13-CD19 is described in SEQ ID NO: 17.

(SEQ ID NO: 17: CDR domains are in bold)
DIQMTQSPSSLSASVGDRVTITCRASQEIRKDLAWYQQKPGKAPKLLIYG
ASSLRSGVPSRFSGSGSGTDFTLTISSLQPEDFATYYCQQDYNYPPTFGC
GTKVEIK

The VL CDR amino acid sequences of 13-CD19 are as follows: RASQEIRKDLA (VL CDR1; SEQ ID NO: 14); GASSLRS (VL CDR2; SEQ ID NO: 15); QQDYNYPPT (VL CDR3; SEQ ID NO: 16).

Summary of CD19 Binding Domains Engineered from 2-CD19

### 14-CD19 Binder:

The heavy chain variable region (VH) amino acid sequence of 14-CD19 is described in SEQ ID NO: 88.

(SEQ ID NO: 88: CDR domains are in bold)
EVQLVQSGAEVKKPGESLKISCKGSGYSFTSYWIGWVRQMPGKCLEWMGI
IYPGDSDTRYSPSFQGQVTISADKSISTAYLQWSSLKASDTAMYYCARGG
TTIFGVVFEYWGQGTLVTVSS

The VH CDR amino acid sequences of 14-CD19 are as follows: SYWIG (VH CDR1; SEQ ID NO:118); IIYPGDSDTRYSPSFQG (VH CDR2; SEQ ID NO: 119); GGTTIFGVVFEY (VH CDR3; SEQ ID NO: 120).

The light chain variable region (V_{L}) amino acid sequence of 14-CD19 is described in SEQ ID NO: 89.

(SEQ ID NO: 89: CDR domains are in bold)
DIQMTQSPSSLSASVGDRVTITCRASQSINNYLNWYQQKPGKAPKLLIYG
ASSLQSGVPSRFSGSGSGTDFTLTISSLQPEDFATYYCQQTYSTPWTFGC
GTKVEIK

The VL CDR amino acid sequences of 14-CD19 are as follows: RASQSINNYLN (VL CDR1; SEQ ID NO: 121); GASSLQS (VL CDR2; SEQ ID NO: 122); QQTYSTPWT (VL CDR3; SEQ ID NO: 123).

The scFv disulfide bond incorporation for both CD19 and CD3 variable domains was accomplished via (i) mutation of variable heavy chain domain framework 2 residue 44 (Kabat numbering) from glycine to cysteine and (ii) mutation of the variable light chain framework 4 residue 100 (Kabat numbering) from glutamine to cysteine [Brinkmann PNAS 1993]. Disulfide bond incorporation was evaluated individually into the CD19 and CD3 binding scFvs in addition to both scFvs having disulfides incorporated to interrogate impacts on production yield following a single step Protein A purification and purity as determined by analytical size exclusion chromatography (SEC) of TCEs.

### Protein A Affinity Chromatography

Protein A affinity capture was performed with a 1 ml HiTrap Mabselect Sure PCC column (Cytiva Life Sciences). The column was equilibrated with 10 column volumes (CVs) of DPBS (1×) (−) calcium chloride, (−) magnesium chloride (Life Technologies). Conditioned medium was then loaded onto the Protein A column at a flow rate of 150 cm/hr. The column load was followed by a 10 CV PBS wash and elution was performed with 100 mM glycine, 150 mM sodium chloride pH 3.0 (additional steps also performed at 150 cm/hr). A 20 milli-absorbance unit (mAu) absorbance threshold was used to direct the product elution to fraction collection. Each well of a 96 deep well collection plate was pre-charged with 50 μl of 1M Tris pH 8.0. Product was collected into the 96 deep well plate in 1 ml fractions and immediately neutralized.

### Analytical Size Exclusion Chromatography (SEC)

SEC analysis was performed utilizing an Agilent 1260 HPLC system (Agilent Technologies) with a Superdex 200 Increase 5/150 GL (5×150 mm) (Cytiva LIfe Sciences). The system and column were operated at room temperature. Approximately 5 μg of sample was injected onto the column equilibrated with a minimum of 10 CVs of DPBS (1×) (−) calcium chloride, (−) magnesium chloride (Life Technologies). Separation was performed over 15 minutes at a flow rate of 0.3 ml/min and UV absorbance was monitored at 280 nm and 214 nm. Yield and purity as measured by analytical SEC data are shown below in Table E12, demonstrating that incorporation of a disulfide bond in both the CD19 and CD3 scFvs results in increased purity of the desired TCE species.

TABLE E12
Purification and purity data for TCEs

| | | Yield | Purity |
|---|---|---|---|
| CD19 scFv | CD3 scFv | mg/L | % target |
| Wild type | Wild type | 42 | 64.1 |
| Disulfide | Wild type | 19 | 98.7 |
| Wild type | Disulfide | 31 | 98.7 |
| Disulfide | Disulfide | 15 | 99.8 |

### Example 13: Hydrophobic Interaction Chromatography (HIC) Analysis of TCEs

The surface hydrophobicity of multiple CD19 TCE molecules having different CD3 and CD19 binders was assessed by hydrophobic interaction chromatography (HIC). Ten micrograms of each sample were loaded on a TSKgel Butyl-NPR column (4.6 mm×3.5 cm, 2.5 m particle size; Tosoh P/N 14947) on an Agilent 1260 Infinity II HPLC system. A linear gradient of mobile phase A (0.1 M sodium phosphate pH 7.0, 2 M ammonium sulfate) and mobile phase B (solution 0.1 M sodium phosphate pH 7.0) was used for 20 minutes at a flow rate of 1.0 ml/min at 25° C. column temperature. HIC retention times (RT) are listed in Table E13. The data demonstrated that most TCEs tested have suitable profiles for downstream development.

TABLE E13
HIC analysis of CD19 × CD3 TCEs

| CD19 | CD3 | HIC RT | HIC % | HIC % | HIC % |
|---|---|---|---|---|---|
| binder | binder | (min) | Pre-peak | Main | Post-peak |
| 2-CD19 | 1-CD3 | 9.931 | 29.030 | 70.980 | −0.010 |
| 2-CD19 | 2-CD3 | 9.913 | 29.840 | 70.160 | 0.000 |
| 2-CD19 | 3-CD3 | 9.932 | 32.030 | 67.960 | 0.010 |
| 2-CD19 | 4-CD3 | 9.941 | 31.040 | 68.960 | 0.000 |
| 1-CD19 | 1-CD3 | 8.938 | 0.000 | 58.470 | 41.530 |
| 1-CD19 | 2-CD3 | 8.914 | 0.000 | 66.870 | 33.130 |
| 1-CD19 | 3-CD3 | 8.936 | 0.000 | 51.190 | 48.810 |
| 1-CD19 | 4-CD3 | 8.948 | 0.000 | 63.570 | 36.430 |

### Example 14. Extracellular Matrix (ECM) ELISA Analysis of TCEs

This Example performed extracellular matrix ELISA analysis of multiple TCEs (generated in the scFv-Fc×Fab-Fc format (i.e., as generally depicted in FIG. 1A) having different CD3 and CD19 binders. Several 96-well Corning Thin-Layer Matrigel Matrix pre-coated ECM plates (Catalog No. 354607) were incubated for one hour at room temperature with 300 μl of blocking buffer (FCS GIBCO; Catalog No. 26010, diluted to 10% in PBS). After incubation, the blocking buffer was removed, and the samples were mixed with fresh blocking buffer in 1:1 ratio in a total volume of 100 μl to yield final concentrations of 1 μM, 0.5 μM, 0.25 μM and 0.05 μM. Six wells had no sample addition for background measurement. After one hour of sample incubation, samples were removed and plates were washed with PBS-T wash buffer 3 times. A 10 ng/ml amount of goat anti-human IgG-HRP conjugated detection antibody (Jackson ImmunoResearch; Catalog No. 109-035-008) was added at 100 μl to all wells. After another one-hour incubation at room temperature, the wells were washed three times with PBS-T wash buffer. After washing, 100 μl of TMB substrate (Surmodics; Catalog No. TMBW-1000-01) was added to each well and allowed to react for 15 minutes, followed by addition of 100 μl of BioFX stop solution (Surmodics; Product code STPR).

Absorbance was then read on a microplate reader at 450 nm referenced at 620 nm. The absorbance values of the samples were divided by the average absorbance value of an internal high control antibody to calculate the % High control values. A lower ECM value was generally desirable because it indicated less non-specific binding to extracellular matrix proteins. A level of 12 as % High control value was chosen as a cutoff since this assay value corresponded to the worst 10% of a set of clinically approved antibodies that were tested using the same protocol. Results for each combination of 1-CD19 and 2-CD19 with varied affinity levels of CD3 binders 1-CD3, 2-CD3, 3-CD3, and 4-CD3 (i.e., each TCE being generated in the scFv-Fc×Fab-Fc format as generally described herein) are shown in Table E14 and demonstrated elevated ECM ELISA binding, suggesting electrostatically dominated non-specific interaction liability (i.e., risk or tendency for proteins to interact with each other due to non-specific electrical charges). Biophysical engineering of 3-CD3 was conducted in order reduce this potential liability while maintaining the desired binding and functional properties of the assembled bispecific construct.

TABLE E14
ECM ELISA analysis of tested TCEs

| | CD3 | Median ECM |
|---|---|---|
| CD19 binder | binder | % high control |
| 2-CD19 | 1-CD3 | 101.61 |
| 2-CD19 | 2-CD3 | 92.18 |
| 2-CD19 | 3-CD3 | 101.65 |
| 2-CD19 | 4-CD3 | 87.98 |
| 1-CD19 | 1-CD3 | 96.9 |
| 1-CD19 | 2-CD3 | 37.13 |
| 1-CD19 | 3-CD3 | 96.74 |
| 1-CD19 | 4-CD3 | 49.35 |

The parental CD3 binder (i.e., 3-CD3; SEQ ID NOs: 40 and 39 for the V_{H} and V_{L}, respectively) demonstrated higher than desired non-specific charged based interactions, as determined by the ECM ELISA assay. Structural analysis for the Fab indicated three charged amino acid positions R16, K58 and K76 that were solvent exposed and expected to be contributing to overall positive surface charge of the molecule. The following mutagenesis strategy was employed to evaluate the biophysical characteristics of single, double and triple amino acid substitutions: R16E or R16G, K58T or K58D, and K76S or K76E. The resultant engineered molecules were evaluated for the ECM ELISA binding in addition to SPR analysis (K_{D}) using CD3ε as an antigen and cellular binding (e.g., EC₅₀).

Biacore C1 Series S sensor (Cytiva) was docked into a Biacore 8K+ instrument and the system was primed with HBS-T+(10 mM HEPES, 150 mM NaCl, 0.05% Tween-20, pH 7.4 buffer (Corning). The C1 chip was conditioned with 0.1 M glycine-NaOH, 0.3% Triton X-100, pH 12 according to manufacturer guidelines. Running buffer was switched to 25 mM MES, pH 5.5, (Alfa Aesar) 0.01% Tween-20 (Thermo Scientific) for the immobilization. The sensor temperature was adjusted to 37° C., and the sample compartment was maintained at 15° C. Upon priming, the chip was activated using a fresh 1:1 mixture of 0.4 M EDC and 0.1 M sulfo-NHS (Thermo Scientific) for seven minutes at 10 μl/ml flow rate across all eight channels. Rabbit anti-mouse IgG polyclonal antibody (Cytiva) was diluted to 50 μg/mL in 10 mM sodium acetate, pH 5.0 (Cytiva) and amine-coupled to all flow cells within each of the eight channels for 15 minutes at 5 μL/min. Finally, to block the activated chip surface, 0.1 M ethylenediamine in 250 mM sodium borate, pH 8.5 buffer was injected over all flow cells within each of the eight channels for 7 minutes at 10 μl/minute. Surfaces were regenerated with two 30-second injections of 10 mM glycine, pH 1.7 (Cytiva) at 30 μL/min.

Engineered CD3-targeting antibodies were measured for their binding affinity to human CD3εδ-muFcG1 in a multi-cycle capture kinetics experiment. Running buffer was switched to HBS-T+(10 mM HEPES, 150 mM NaCl, 0.05% Tween-20, pH 7.4 buffer (Corning) with 0.1% BSA (Sigma-Aldrich) and the system was thoroughly primed in this buffer. Human CD3εδ-muFcG1 was diluted to 15 μg/mL in running buffer and served as ligand. Five-point, five-fold serial dilutions of each monovalent bispecific CD3-targeting antibody at 100 nM top concentration were prepared in running buffer. Three startup cycles, which are abbreviated analysis cycles of shortened injections but with complete regeneration, were introduced prior to the analysis experiment. In each analysis cycle, human CD3εδ-muFcG1was captured as ligand to only flow cell 2 of each channel at 5 μL/min for 2 min. Flow cell 1 of each channel remained unaddressed and served as the reference surface. A two-minute stabilization period was introduced after every ligand capture. A single analyte sample was injected per cycle over each channel for 2 min at 30 μL/min with 10 min dissociation. Analyte samples comprised of the monovalent, bispecific CD3-targeting antibody serial dilutions and were injected in order from lowest to highest concentration with buffer blanks flanking the concentration series. Amine-coupled rabbit anti-mouse IgG polyclonal antibody surfaces were regenerated at the end of each cycle with one injection of 10 mM glycine, pH 1.7 at 30 μL/min for one minute.

Data are shown in Table E15. The combination of point mutations R16G and K58T significantly reduced the observed ECM ELISA liability while maintaining similar binding affinity (K_{D}). These mutants, in conjunction with the disulfide introduction in the scFv (as described in Example 12), were used to generate 5-CD3.

TABLE E15
Biophysical analysis of CD3 binders

| | ECM % High | | | | | | |
|---|---|---|---|---|---|---|---|
| | 1 | 0.2 | 0.04 | KD | | | |
| | μM | μM | μM | (nM) | R16 | K58 | K76 |
| Parental | 65.8 | 24.7 | 8.5 | 4.3 | R | K | K |
| 3-CD3 | | | | | | | |
| 9-CD3 | 36.1 | 12.7 | 5.6 | 5.0 | G | K | K |
| 10-CD3 | 16.3 | 8.2 | 5.3 | 5.5 | E | K | K |
| 6-CD3 | 7.1 | 5.3 | 4.8 | 12 | G | T | K |
| 11-CD3 | 5.6 | 4.8 | 5.0 | 20 | E | D | K |
| 12-CD3 | 7.2 | 6.1 | 5.6 | >50 | G | T | S |
| 13-CD3 | 5.3 | 4.8 | 3.8 | >50 | E | D | E |
| 14-CD3 | 6.5 | 4.7 | 4.1 | 9.7 | G | K | S |
| 15-CD3 | 9.6 | 6.3 | 5.6 | >50 | E | K | E |
| low control | 3.8 | | | | | | |
| high control | 100.0 | | | | | | |

A summary of the 5-CD3 through 16-CD3 binding domains is provided below.

### 5-CD3 Binder

The heavy chain variable region (VH) amino acid sequence of 5-CD3 is described in SEQ ID NO: 22 (note: VH amino acid changes when compared to the VH of 1-CD3 binder (i.e., SEQ ID NO: 36) are R16G, K58T, G44C, G55A, D80Y, I112L, N113D).

(SEQ ID NO: 22; CDR domains are in bold)
QVQLVESGGGVVQPGGSLRLSCAASGFTFSRYGIHWVRQAPGKCLEWVAA
IWYDASKTYYADSVKGRFTISRDNSKNTLYLQMNSLRAEDTAVYYCARSN
YDILTRYYQYGLDVWGQGTTVTVSS

The VH CDR amino acid sequences of 5-CD3 are as follows: RYGIH (VH CDR1; SEQ ID NO: 19); AIWYDASKTYYADSVKG (VH CDR2; SEQ ID NO: 20); SNYDILTRYYQYGLDV (VH CDR3; SEQ ID NO: 21).

The light chain variable region (VL) amino acid sequence of 5-CD3 is described in SEQ ID NO: 27 (note: VL amino acid changes when compared to the VL of 1-CD3 binder (i.e., SEQ ID NO: 37) are N56S, Q100C).

(SEQ ID NO: 27: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGC
GTKVEIK

The VL CDR amino acid sequences of 5-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 6-CD3 Binder

The heavy chain variable region (VH) amino acid sequence of 6-CD3 is described in SEQ ID NO: 68 (note: VH amino acid changes when compared to the VH of 1-CD3 binder (i.e., SEQ ID NO: 36) are R16G, K58T, G55A, D80Y, I112L, N113D)

(SEQ ID NO: 68; CDR domains are in bold)
QVQLVESGGGVVQPGGSLRLSCAASGFTFSRYGIHWVRQAPGKGLEWVAA
IWYDASKTYYADSVKGRFTISRDNSKNTLYLQMNSLRAEDTAVYYCARSN
YDILTRYYQYGLDVWGQGTTVTVSS

The VH CDR amino acid sequences of 6-CD3 are as follows: RYGIH (VH CDR1; SEQ ID NO: 19); AIWYDASKTYYADSVKG (VH CDR2; SEQ ID NO: 20); SNYDILTRYYQYGLDV (VH CDR3; SEQ ID NO: 21).

The light chain variable region (VL) amino acid sequence of 6-CD3 is described in SEQ ID NO: 39 (note: VL amino acid change when compared to the VL of 1-CD3 binder (i.e., SEQ ID NO: 37) is N56S).

(SEQ ID NO: 39: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGQ
GTKVEIK

The VL CDR amino acid sequences of 6-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 7-CD3 Binder

The heavy chain variable region (VH) amino acid sequence of 7-CD3 is described in SEQ ID NO: 70 (note: VH amino acid changes when compared to the VH of 1-CD3 binder (i.e., SEQ ID NO: 36) are R16G, K58T, G55A, D80Y, Q109S, Y110H)

(SEQ ID NO: 70; CDR domains are in bold)
QVQLVESGGGVVQPGGSLRLSCAASGFTFSRYGIHWVRQAPGKGLEWVAA
IWYDASKTYYADSVKGRFTISRDNSKNTLYLQMNSLRAEDTAVYYCARSN
YDILTRYYSHGINVWGQGTTVTVSS

The VH CDR amino acid sequences of 7-CD3 are as follows: RYGIH (VH CDR1; SEQ ID NO: 19); AIWYDASKTYYADSVKG (VH CDR2; SEQ ID NO: 20); SNYDILTRYYSHGINV (VH CDR3; SEQ ID NO: 111).

The light chain variable region (VL) amino acid sequence of 7-CD3 is described in SEQ ID NO: 43 (note: VL amino acid change when compared to the VL of 1-CD3 binder (i.e., SEQ ID NO: 37) is N56S and K92N).

(SEQ ID NO: 43: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFNSYPRTFGQ
GTKVEIK

The VL CDR amino acid sequences of 7-CD3 are as follows: RASQGISSALA (VL CDR1; SEQ ID NO: 24); DASSLES (VL CDR2; SEQ ID NO: 25); QQFNSYPRT (VL CDR3; SEQ ID NO: 115).

### 8-CD3 Binder

The heavy chain variable region (VH) amino acid sequence of 8-CD3 is described in SEQ ID NO: 72 (note: VH amino acid changes when compared to the VH of 1-CD3 binder (i.e., SEQ ID NO: 36) are R16G, K58T, G44C, G55A, D80Y, Q109S, Y110H)

(SEQ ID NO: 72; CDR domains are in bold)
QVQLVESGGGVVQPGGSLRLSCAASGFTFSRYGIHWVRQAPGKCLEWVAA
IWYDASKTYYADSVKGRFTISRDNSKNTLYLQMNSLRAEDTAVYYCARSN
YDILTRYYSHGINVWGQGTTVTVSS

The VH CDR amino acid sequences of 8-CD3 are as follows: RYGIH (VH CDR1; SEQ ID NO: 19); AIWYDASKTYYADSVKG (VH CDR2; SEQ ID NO: 20); SNYDILTRYYSHGINV (VH CDR3; SEQ ID NO: 111).

The light chain variable region (VL) amino acid sequence of 8-CD3 is described in SEQ ID NO: 73 (note: VL amino acid change when compared to the VL of 1-CD3 binder (i.e., SEQ ID NO: 37) is N56S, K92N and Q100C).

(SEQ ID NO: 73: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFNSYPRTFGC
GTKVEIK

The VL CDR amino acid sequences of 8-CD3 are as follows: RASQGISSALA (VL CDR1; SEQ ID NO: 24); DASSLES (VL CDR2; SEQ ID NO: 25); QQFNSYPRT (VL CDR3; SEQ ID NO: 115).

### 9-CD3 Binder

The heavy chain variable region (VH) amino acid sequence of 9-CD3 is described in SEQ ID NO: 74 (note: VH amino acid changes when compared to the VH of 1-CD3 binder (i.e., SEQ ID NO: 36) are R16G, G55A, D80Y, I112L, N113D).

(SEQ ID NO: 74; CDR domains are in bold)
QVQLVESGGGVVQPGGSLRLSCAASGFTFSRYGIHWVRQAPGKGLEWVAA
IWYDASKKYYADSVKGRFTISRDNSKNTLYLQMNSLRAEDTAVYYCARSN
YDILTRYYQYGLDVWGQGTTVTVSS

The VH CDR amino acid sequences of 9-CD3 are as follows: RYGIH (VH CDR1; SEQ ID NO: 19); AIWYDASKKYYADSVKG (VH CDR2; SEQ ID NO: 110); SNYDILTRYYQYGLDV (VH CDR3; SEQ ID NO: 21).

The light chain variable region (VL) amino acid sequence of 9-CD3 is described in SEQ ID NO: 39 (note: VL amino acid change when compared to the VL of 1-CD3 binder (i.e., SEQ ID NO: 37) is N56S).

(SEQ ID NO: 39: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGQ
GTKVEIK

The VL CDR amino acid sequences of 9-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 10-CD3 Binder

The heavy chain variable region (VH) amino acid sequence of 10-CD3 is described in SEQ ID NO: 195 (note: VH amino acid changes when compared to the VH of 1-CD3 binder (i.e., SEQ ID NO: 36) are R16E, G55A, D80Y, I112L, N113D)

(SEQ ID NO: 195; CDR domains are in bold)
QVQLVESGGGVVQPGESLRLSCAASGFTFSRYGIHWVRQAPGKGLEWVA
AIWYDASKKYYADSVKGRFTISRDNSKNTLYLQMNSLRAEDTAVYYCAR
SNYDILTRYYQYGLDVWGQGTTVTVSS

The VH CDR amino acid sequences of 10-CD3 are as follows: RYGIH (VH CDR1; SEQ ID NO: 19); AIWYDASKKYYADSVKG (VH CDR2; SEQ ID NO: 110); SNYDILTRYYQYGLDV (VH CDR3; SEQ ID NO: 21).

The light chain variable region (VL) amino acid sequence of 10-CD3 is described in SEQ ID NO: 39 (note: VL amino acid change when compared to the VL of 1-CD3 binder (i.e., SEQ ID NO: 37) is N56S).

(SEQ ID NO: 39: CDR domains are in bold)
AIQLTQSPSSLSASVGDRVTITCRASQGISSALAWYQQKPGKAPKLLIYD
ASSLESGVPSRFSGSGSGTDFTLTISSLQPEDFATYYCQQFKSYPRTFGQ
GTKVEIK

The VL CDR amino acid sequences of 10-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 11-CD3 Binder

The heavy chain variable region (VH) amino acid sequence of 11-CD3 is described in SEQ ID NO: 78 (note: VH amino acid changes when compared to the VH of 1-CD3 binder (i.e., SEQ ID NO: 36) are R16E, K58D, G55A, D80Y, I112L, N113D)

(SEQ ID NO: 78; CDR domains are in bold)
QVQLVESGGGVVQPGESLRLSCAASGFTFSRYGIHWVRQAPGKGLEWVA
AIWYDASKDYYADSVKGRFTISRDNSKNTLYLQMNSLRAEDTAVYYCAR
SNYDILTRYYQYGLDVWGQGTTVTVSS

The VH CDR amino acid sequences of 11-CD3 are as follows: RYGIH (VH CDR1; SEQ ID NO: 19); AIWYDASKDYYADSVKG (VH CDR2; SEQ ID NO: 112); SNYDILTRYYQYGLDV (VH CDR3; SEQ ID NO: 21).

The light chain variable region (VL) amino acid sequence of 11-CD3 is described in SEQ ID NO: 39 (note: VL amino acid change when compared to the VL of 1-CD3 binder (i.e., SEQ ID NO: 37) is N56S).

(SEQ ID NO: 39: CDR domains are in bold)
AIQLTQSPSSLSASVGDRVTITCRASQGISSALAWYQQKPGKAPKLLIY
DASSLESGVPSRFSGSGSGTDFTLTISSLQPEDFATYYCQQFKSYPRTF
GQGTKVEIK

The VL CDR amino acid sequences of 11-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 12-CD3 Binder

The heavy chain variable region (VH) amino acid sequence of 12-CD3 is described in SEQ ID NO: 80 (note: VH amino acid changes when compared to the VH of 1-CD3 binder (i.e., SEQ ID NO: 36) are R16G, K58T, K76S, G55A, D80Y, I112L, N113D)

(SEQ ID NO: 80; CDR domains are in bold)
QVQLVESGGGVVQPGGSLRLSCAASGFTFSRYGIHWVRQAPGKGLEWVA
AIWYDASKTYYADSVKGRFTISRDNSSNTLYLQMNSLRAEDTAVYYCAR
SNYDILTRYYQYGLDVWGQGTTVTVSS

The VH CDR amino acid sequences of 12-CD3 are as follows: RYGIH (VH CDR1; SEQ ID NO: 19); AIWYDASKTYYADSVKG (VH CDR2; SEQ ID NO: 20); SNYDILTRYYQYGLDV (VH CDR3; SEQ ID NO: 21).

The light chain variable region (VL) amino acid sequence of 12-CD3 is described in SEQ ID NO: 39 (note: VL amino acid change when compared to the VL of 1-CD3 binder (i.e., SEQ ID NO: 37) is N56S).

(SEQ ID NO: 39: CDR domains are in bold)
AIQLTQSPSSLSASVGDRVTITCRASQGISSALAWYQQKPGKAPKLLIY
DASSLESGVPSRFSGSGSGTDFTLTISSLQPEDFATYYCQQFKSYPRTF
GQGTKVEIK

The VL CDR amino acid sequences of 12-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 13-CD3 Binder

The heavy chain variable region (VH) amino acid sequence of 13-CD3 is described in SEQ ID NO: 82 (note: VH amino acid changes when compared to the VH of 1-CD3 binder (i.e., SEQ ID NO: 36) are R16E, K58D, K76S, G55A, D80Y, I112L, N113D)

(SEQ ID NO: 82; CDR domains are in bold)
QVQLVESGGGVVQPGESLRLSCAASGFTFSRYGIHWVRQAPGKGLEWVA
AIWYDASKDYYADSVKGRFTISRDNSENTLYLQMNSLRAEDTAVYYCAR
SNYDILTRYYQYGLDVWGQGTTVTVSS

The VH CDR amino acid sequences of 13-CD3 are as follows: RYGIH (VH CDR1; SEQ ID NO: 19); AIWYDASKDYYADSVKG (VH CDR2; SEQ ID NO: 112); SNYDILTRYYQYGLDV (VH CDR3; SEQ ID NO: 21).

The light chain variable region (VL) amino acid sequence of 13-CD3 is described in SEQ ID NO: 39 (note: VL amino acid change when compared to the VL of 1-CD3 binder (i.e., SEQ ID NO: 37) is N56S).

(SEQ ID NO: 39: CDR domains are in bold)
AIQLTQSPSSLSASVGDRVTITCRASQGISSALAWYQQKPGKAPKLLIY
DASSLESGVPSRFSGSGSGTDFTLTISSLQPEDFATYYCQQFKSYPRTF
GQGTKVEIK

The VL CDR amino acid sequences of 13-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 14-CD3 Binder

The heavy chain variable region (VH) amino acid sequence of 14-CD3 is described in SEQ ID NO: 84 (note: VH amino acid changes when compared to the VH of 1-CD3 binder (i.e., SEQ ID NO: 36) are R16G, K76S, G55A, D80Y, I112L, N113D)

(SEQ ID NO: 84; CDR domains are in bold)
QVQLVESGGGVVQPGGSLRLSCAASGFTFSRYGIHWVRQAPGKGLEWVA
AIWYDASKKYYADSVKGRFTISRDNSSNTLYLQMNSLRAEDTAVYYCAR
SNYDILTRYYQYGLDVWGQGTTVTVSS

The VH CDR amino acid sequences of 14-CD3 are as follows: RYGIH (VH CDR1; SEQ ID NO: 19); AIWYDASKKYYADSVKG (VH CDR2; SEQ ID NO: 110); SNYDILTRYYQYGLDV (VH CDR3; SEQ ID NO: 21).

The light chain variable region (VL) amino acid sequence of 14-CD3 is described in SEQ ID NO: 39 (note: VL amino acid change when compared to the VL of 1-CD3 binder (i.e., SEQ ID NO: 37) is N56S).

(SEQ ID NO: 39: CDR domains are in bold)
AIQLTQSPSSLSASVGDRVTITCRASQGISSALAWYQQKPGKAPKLLIY
DASSLESGVPSRFSGSGSGTDFTLTISSLQPEDFATYYCQQFKSYPRTF
GQGTKVEIK

The VL CDR amino acid sequences of 14-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 15-CD3 Binder

The heavy chain variable region (VH) amino acid sequence of 15-CD3 is described in SEQ ID NO: 86 (note: VH amino acid changes when compared to the VH of 1-CD3 binder (i.e., SEQ ID NO: 36) are R16E, K76E, G55A, D80Y, I112L, N113D)

(SEQ ID NO: 86; CDR domains are in bold)
QVQLVESGGGVVQPGESLRLSCAASGFTFSRYGIHWVRQAPGKGLEWVA
AIWYDASKKYYADSVKGRFTISRDNSENTLYLQMNSLRAEDTAVYYCAR
SNYDILTRYYQYGLDVWGQGTTVTVSS

The VH CDR amino acid sequences of 15-CD3 are as follows: RYGIH (VH CDR1; SEQ ID NO: 19); AIWYDASKKYYADSVKG (VH CDR2; SEQ ID NO: 110); SNYDILTRYYQYGLDV (VH CDR3; SEQ ID NO: 21).

The light chain variable region (VL) amino acid sequence of 15-CD3 is described in SEQ ID NO: 39 (note: VL amino acid change when compared to the VL of 1-CD3 binder (i.e., SEQ ID NO: 37) is N56S).

(SEQ ID NO: 39: CDR domains are in bold)
AIQLTQSPSSLSASVGDRVTITCRASQGISSALAWYQQKPGKAPKLLIY
DASSLESGVPSRFSGSGSGTDFTLTISSLQPEDFATYYCQQFKSYPRTF
GQGTKVEIK

The VL CDR amino acid sequences of 15-CD3 are as follows: RASQGISSALA (VL CDR1; SEQ ID NO: 24); DASSLES (VL CDR2; SEQ ID NO: 25); QQFKSYPRT (VL CDR3; SEQ ID NO: 26).

### 16-CD3 Binder

The heavy chain variable region (VH) amino acid sequence of 16-CD3 is described in SEQ ID NO: 159 (note: VH amino acid changes when compared to the VH of 1-CD3 binder (i.e., SEQ ID NO: 36) are R16G, K58T, G55A, D80Y, R106G, Y107H)

(SEQ ID NO: 159; CDR domains are in bold)
QVQLVESGGGVVQPGGSLRLSCAASGFTFSRYGIHWVRQAPGKGLEWVA
AIWYDASKTYYADSVKGRFTISRDNSKNTLYLQMNSLRAEDTAVYYCAR
SNYDILTGHYQYGINVWGQGTTVTVSS

The VH CDR amino acid sequences of 16-CD3 are as follows: RYGIH (VH CDR1; SEQ ID NO: 19); AIWYDASKTYYADSVKG (VH CDR2; SEQ ID NO: 20); SNYDILTGHYQYGINV (VH CDR3; SEQ ID NO: 113).

The light chain variable region (VL) amino acid sequence of 16-CD3 is described in SEQ ID NO: 43 (note: VL amino acid change when compared to the VL of 1-CD3 binder (i.e., SEQ ID NO: 37) is N56S and K92N).

(SEQ ID NO: 43: CDR domains are in bold)
AIQLTQSPSSLSASVGDRVTITCRASQGISSALAWYQQKPGKAPKLLIY
DASSLESGVPSRFSGSGSGTDFTLTISSLQPEDFATYYCQQFNSYPRTF
GQGTKVEIK

The VL CDR amino acid sequences of 16-CD3 are as follows: RASQGISSALA (VL CDR1; SEQ ID NO: 24); DASSLES (VL CDR2; SEQ ID NO: 25); QQFNSYPRT (VL CDR3; SEQ ID NO: 115).

### Example 15: Human PBMC Depletion Assay Using CD19×CD3 TCEs

This Example analyzed the ability of the TCEs to effectuate human B cell depletion and cytokine secretion. Cryopreserved human peripheral blood mononuclear cells (PBMCs) from healthy donors were obtained from StemCell Technologies. PBMCs were thawed, washed, and resuspended to a concentration of approximately 1.1×10⁶ cells/mL in cell culture media containing RPMI (Gibco; Catalog No. 11875093), 10% HI FBS (Gibco; Catalog No. 10062-147), and 1% penicillin/streptomycin (Gibco; Catalog No. 15140122). A 9 point four-fold concentration titration of TCE was prepared in culture media by preparing 10× top concentration of TCE and then diluting four-fold in culture media. A 20 μL volume of 10×TCE dilutions was plated per well of a low-bind round bottom 96 well plate (Corning #7007). Approximately 200,000 PBMCs per well (180 μL) were added on top of the TCE and the plates were incubated for approximately 72 hours at 37° C., 5% CO₂.

After approximately 72 hours, flow cytometry analysis was performed to assess B cell depletion. Cells were transferred to a 96 well v-bottom plate for staining and pelleted by centrifugation. The supernatants were collected in a 96 well, low-bind round bottom plate (Corning #7007) and stored at −80° C. for cytokine analysis. Cells were washed in 200 μL PBS (ThermoFisher Scientific; Catalog No. 10010023), centrifuged to pellet cells and any supernatant was discarded. Then 200 μL of fixable viability dye (BD; Catalog No. 565388) diluted 1:1000 in PBS was added to each well, followed by incubation for 20 minutes at ambient temperature in the dark. Samples were pelleted by centrifugation and supernatant was discarded. Samples were washed by adding 200 μL of FACs buffer comprised of PBS and 2% FBS (fetal bovine serum, certified, heat inactivated, ThermoFisher Scientific; Catalog No. 10082147), pelleted by centrifugation, and supernatant was discarded. For surface antigen staining, samples were resuspended in 100 μL of a surface antigen staining reagent as described in Table E16 followed by incubation for 30 minutes in the dark at ambient temperature.

TABLE E16
Human PBMC Surface antigen flow cytometry antibody panel

| | | | | | volume |
|---|---|---|---|---|---|
| | | | | | per |
| | | | | | sample |
| Vendor | Catalog# | Clone | Marker | Fluorophore | (μL) |
| Biolegend | 612817 | FN50 | CD69 | BUV737 | 1 |
| BD | 612942 | RPA-T8 | CD8 | BUV496 | 1 |
| BD | 563550 | SK3 | CD4 | BUV395 | 1 |
| Biolegend | 302356 | 2H7 | CD20 | BV786 | 1 |
| Biolegend | 356138 | M-A251 | CD25 | BV711 | 1 |
| BD | 740594 | IA6-2 | IgD | BV650 | 1 |
| Biolegend | 301842 | M5E2 | CD14 | BV510 | 1 |
| BD | 568219 | B159 | CD56 | BV421 | 1 |
| Invitrogen | 46-1388-42 | MI15 | CD138 | PerCP-Cy5.5 | 1 |
| BD | 560994 | HIB19 | CD19 | FITC | 2 |
| eBio | 25-0271-82 | LG.7F9 | CD27 | PE-Cy7 | 1 |
| Biolegend | 317346 | OKT3 | CD3 | PE-Dazzle594 | 1 |
| Biolegend | 357504 | 19F2 | BCMA (CD269) | PE | 1 |
| Biolegend | 356606 | HB7 | CD38 | APC | 1 |
| BD | 564220 | | FC block | human | 1 |
| BD | 566385 | | Brilliant stain | | 10 |
| | | | buffer plus | | |
| | | | PBS | | 74 |

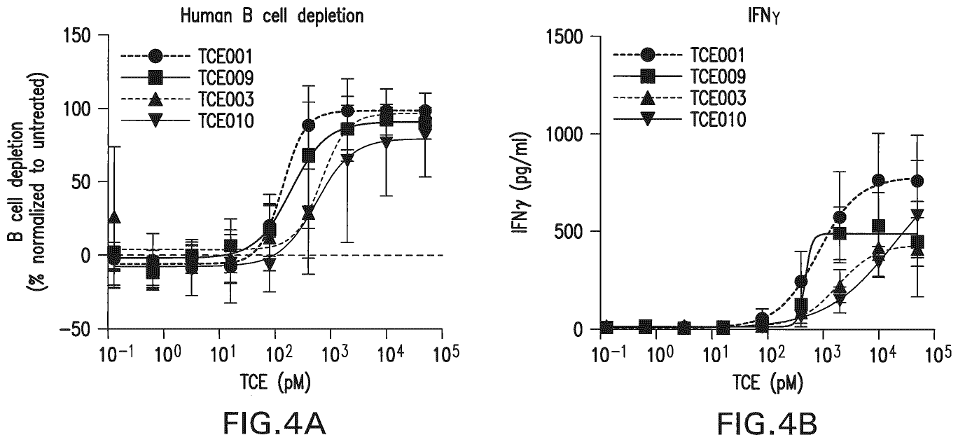

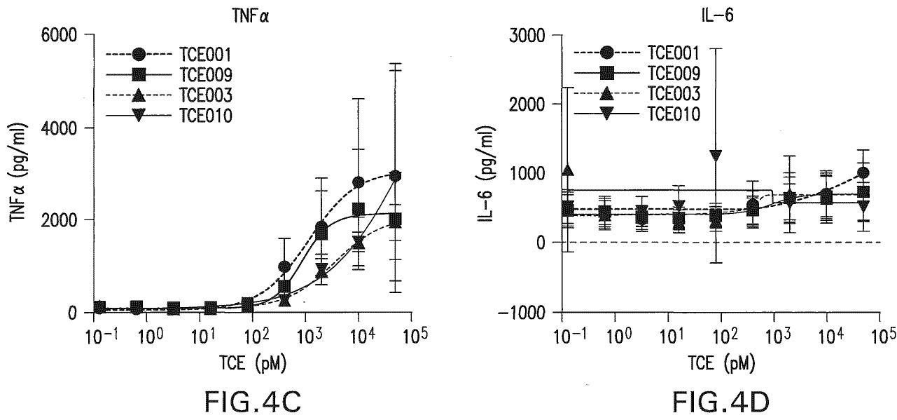

Following incubation, samples were pelleted by centrifugation, supernatant was discarded, and 200 μL FACs buffer was added to wash cells. Samples were again centrifuged to pellet cells, supernatant was discarded, and samples were resuspended in fixation buffer (eBioscience™ IC Fixation Buffer, ThermoFisher Scientific; Catalog No. 00-8222-49, used as a 2× solution, diluted at 1:2 in distilled water (ThermoFisher Scientific; Catalog No. 15230162), followed by incubation for 10 minutes in the dark at ambient temperature to fix cells. Samples were washed twice by pelleting cells, discarding supernatant, and resuspending in 200 μL PBS. After the final wash, samples were pelleted by centrifugation and resuspended in 120 μL of PBS for multicolor flow cytometry analysis on a BD LSRFortessa cell analyzer. The CD20+ B cell counts were calculated and normalized to untreated control wells to quantify percentage depletion of B cells in response to TCE treatment. A panel of TCEs (TCE001, TCE009, TCE003, TCE010; all with the RFHis modification described in Example 9) containing the CD19 binder 13-CD19 and 4 different CD3 binders (specifically, 5-CD3, 8-CD3, 6-CD3, and 7-CD3) in both the scFv-Fc×Fab-Fc and the scFv-Fc×scFv-Fc format was tested in this assay. Depletion EC₅₀ values and depletion Eₘₐₓ (top parameter of the curve) were estimated by fitting concentration response data using nonlinear regression with a four-parameter logistic model. Data in Table E17 and FIG. 4A demonstrated that each TCE (i.e., TCE001, TCE009, TCE003 and TCE010) was effective in depleting B cells in the assay.

For example, as reported in Table E17, CD3 binders delivered profound and potent B cell depletion when paired with CD19 binder 13-CD19, with an EC₅₀ of 136 μM in the scFv-Fc×scFv-Fc format (5-CD3) and an EC₅₀ of 196 μM in the scFv-Fc×Fab-Fc format (6-CD3). These data indicated that TCE001 showed the most favorable Eₘₐₓ and EC₅₀. In addition, the scFv-Fc×scFv-Fc format improved Eₘₐₓ for TCE001 and TCE003, with the 5-CD3 binder demonstrating greater potency than the 8-CD3 binder.

TABLE E17
Effect of different CD3 binders on B cell depletion potency
and efficacy in a human PBMC assay

| | | | | Depletion | |
|---|---|---|---|---|---|
| | CD19 | CD3 | | Eₘₐₓ | EC₅₀ |
| TCE id. | binder | binder | TCE format | (%) | (pM) |
| TCE001 | 13-CD19 | 5-CD3 | (scFv-Fc) × (scFv-Fc) | 99 | 136 |
| TCE003 | 13-CD19 | 8-CD3 | (scFv-Fc) × (scFv-Fc) | 97 | 675 |
| TCE009 | 13-CD19 | 6-CD3 | (scFv-Fc) × (Fab-Fc) | 91 | 196 |
| TCE010 | 13-CD19 | 7-CD3 | (scFv-Fc) × (Fab-Fc) | 80 | 555 |

Cytokine secretion in the supernatants was assessed by using a Human ProcartaPlex Mix&Match 4-plex kit (ThermoFisher; Catalog No. PPX-04, Lot #4408187-000). Samples were thawed on ice and then centrifuged at 10,000×g for 5 minutes to pellet out precipitate. The manufacturer's recommended protocol for analyte detection was followed. Data were acquired on Luminex xMAP INTELLIFLEX instrument system (ThermoFisher; Catalog No. APX2020). EC₅₀ values for cytokine induction were calculated by fitting the concentration-response data using nonlinear regression with a four-parameter logistic model. Instances where the curve did not converge or lacked a clear concentration-dependent response were indicated as “ND” in Tables E18, E19, and E20. Concentration-dependent induction of IFNγ (FIG. 4B) and TNFα (FIG. 4C) was observed in response to each TCE evaluated, and IL-6 induction (FIG. 4D) was observed for TCE009. Where calculable, the potency (EC₅₀) of cytokine induction (Table E18, Table E19, and Table E20) by TCEs (i.e., TCE001, TCE009, TCE003 and TCE010) was reduced compared to the potency of B cell depletion (Table E17).

TABLE E18
Concentration dependent induction of IFNγ by TCEs

| | TCE001 | TCE009 | TCE003 | TCE010 |
|---|---|---|---|---|
| EC₅₀ (pM) | 812 | 471 | 1811 | ND* |

*ND = EC₅₀ could not be determined due to non-convergence of the curve fit

TABLE E19
Concentration dependent induction of TNFα by TCEs

| | TCE001 | TCE009 | TCE003 | TCE010 |
|---|---|---|---|---|
| EC₅₀ (pM) | 1162 | 865 | 3528 | ND* |

*ND = EC₅₀ could not be determined due to non-convergence of the curve fit

TABLE E20
Concentration dependent induction of IL-6 by TCEs

| | TCE001 | TCE009 | TCE003 | TCE010 |
|---|---|---|---|---|
| EC₅₀ (pM) | ND | 916 | ND* | ND* |

*ND = EC₅₀ could not be determined due to either non-convergence of the curve fit or absence of a clear concentration-dependent response

Consistent with the inventors' hypothesis, these data demonstrated the potential for clinically relevant decoupling of specific killing from cytokine release by TCEs, which may indicate an enhanced clinical safety profile.

### Example 16: Epitopes and Paratopes for the Parental 1-CD3 Anti-CD3 Epsilon Antibody

The crystal structure of an antigen-binding fragment of 1-CD3 in complex with a human CD3 epsilon peptide “ZDGNEEMGGITQTP” (SEQ ID NO: 240) where Z represents pyroglutamic acid (PCA), a chemically modified version of Q, was determined at 2.8 Å. A “Protein interfaces, surfaces and assemblies” (PISA) analysis of the structure was used to determine the residues of 1-CD3 interacting with the CD3 epsilon peptide (European Bioinformatics Institute, http://www.ebi.ac.uk/pdbe/prot_int/pistart.html, Krissinel and Henrick (2007) J. Mol. Biol. 372, 774-797.) Based on this analysis, residues Z1, D2, G3, N4, E5, E6, M7 are interacting with the heavy chain of 1-CD3, and residues Z1, D2, E5, E6 are interacting with the light chain of 1-CD3.

Paratope residues that are within 4.5 Å of the CD3 antigen in the 1-CD3 antibody/antigen complex were identified. The key paratope residues for human CD3 epsilon in the 1-CD3 antibody were determined to be residues G33, H35, 151, W52, Y53, S99, Y101, 1103, R106, Q109, and Y110 in the 1-CD3 heavy chain variable region (SEQ ID NO: 36) and residues S30, F91, K92, Y94, and R96 in the 1-CD3 light chain variable region (SEQ ID NO:37).

The binding epitope for an exemplary bispecific antibody with the antigen-binding fragment of 1-CD3 in complex with a human CD3 epsilon recombinant protein was further characterized by hydrogen-deuterium exchange, fast photochemical oxidation of proteins (FPOP) and glycine ethyl ester (GEE) labeling mass spectrometry.

Based on these analyses, E5, E6 and M7 in the N-terminal region of mature human CD3 epsilon (SEQ ID NO:5 in Table E21) were shown to interact with 1-CD3.

TABLE E21
Sequence of mature human CD3 epsilon

| | | |
|---|---|---|
| Mature | QDGNEEMGGITQTPYKVSISGTTVILTCPQYPGSEILWQH | SEQ ID NO: 5 |
| human | NDKNIGGDEDDKNIGSDEDHLSLKEFSELEQSGYYVCYP | |
| CD3 | RGSKPEDANFYLYLRARVCENCMEMDVMSVATIVIVDIC | |
| epsilon | ITGGLLLLVYYWSKNRKAKAKPVTRGAGAGGRQRGQNK | |
| | ERPPPVPNPDYEPIRKGQRDLYSGLNQRRI | |

### Example 17: Non-Human Primate PBMC Depletion Assay Using CD19×CD3 TCEs

This Example analyzed the ability of TCEs to deplete B cells in non-human primate PBMCs. Cryopreserved cynomolgus macaque PBMCs from 3 donors were obtained from Alpha Genesis. PBMCs were thawed, washed, and resuspended to a concentration of approximately_1.1×10⁶ cells/mL in cell culture media containing RPMI (Gibco; Catalog No. 11875093), 10% HI FBS (Gibco; Catalog No. 10062-147), and 1% penicillin/streptomycin (Gibco; Catalog No. 15140122). A nine point five-fold concentration titration of TCE (i.e. TCE001, TCE009, TCE003, TCE010; all with the RFHis modification as described in Example 9) was prepared in RPMI culture media by preparing 10× top concentration of TCE and then diluting five-fold in culture media. A 20 μL volume of 10×TCE dilutions was plated per well of a low-bind, round bottom 96 well plate (Corning #7007). Approximately 200,000 PBMCs per well (180 μL volume) were added on top of the TCE and the plates were incubated for approximately 72 hours at 37° C., and 5% CO₂.

After approximately 72 hours, flow cytometry was performed to assess B cell depletion. Cells were transferred to a 96 well v-bottom plate for staining, centrifuged to pellet cells followed by decanting of supernatants which were collected in a 96 well, low-bind round bottom plate and stored at −80° C. for cytokine analysis. Cells were washed in 200 μL PBS, ThermoFisher Scientific; Catalog No. 10010023), centrifuged to pellet cells and supernatant was discarded. A 200 μL volume of fixable viability dye (BD; Catalog No. 565388) diluted 1:1000 in PBS was added to each well, followed by incubation for 20 minutes at ambient temperature in the dark. Samples were pelleted by centrifugation and supernatant was discarded. Samples were washed by adding 200 μL of FACs buffer [PBS containing 2% of FBS (Fetal Bovine Serum, certified, heat inactivated, ThermoFisher Scientific; Catalog No. 10082147)], pelleted by centrifugation, and supernatant was discarded. For surface antigen staining, samples were resuspended in 100 μL of surface antigen staining reagents as described in Table E22 followed by incubation for 30 minutes in the dark at ambient temperature.

TABLE E22
Cyno PBMC Surface antigen flow cytometry antibody panel

| | | | | | volume |
|---|---|---|---|---|---|
| | | | | | per |
| | | | | | sample |
| Vendor | Catalog# | Clone | Marker | Fluorophore | (μL) |
| BD ™ | 612848 | 2H7 | CD20 | BUV737 | 1 |
| BD ™ | 612944 | 3G8 | CD16 | BUV496 | 1 |
| BD ™ | 564117 | SP34-2 | CD3 | BUV395 | 2 |
| Biolegend ™ | 305442 | IT2.2 | CD86 | BV786 | 2 |
| BD ™ | 563163 | B-Ly4 | CD21 | BV711 | 2 |
| BD ™ | 563737 | L200 | CD4 | BV650 | 1 |
| BD ™ | 562989 | FN50 | CD69 | BV605 | 2 |
| Biolegend ™ | 301842 | M5E2 | CD14 | BV510 | 1 |
| Biolegend ™ | 301036 | RPA-T8 | CD8a | BV421 | 2 |
| BD ™ | 560842 | B159 | CD56 | PerCP-Cy5.5 | 1 |
| StemCell ™ | 60131FI | AT-1 | CD38 | FITC | 1 |
| FisherScientific ™ | NC0445617 | Z199 | NKG2A | PE-Cy7 | 1 |
| | | | (CD159a) | | |
| Biolegend ™ | 356422 | M-T271 | CD27 | PE/Dazzle594 | 1 |
| Biolegend ™ | 352314 | DL-101 | CD138 | AF647 | 1 |
| BD | 564220 | | FC block | human | 1 |
| BD | 566385 | | Brilliant stain | | 10 |
| | | | plus | | |
| | | | PBS | | 70 |

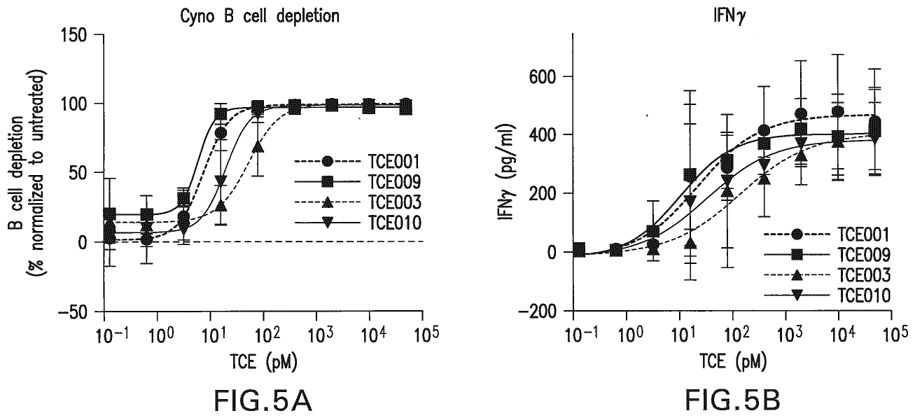

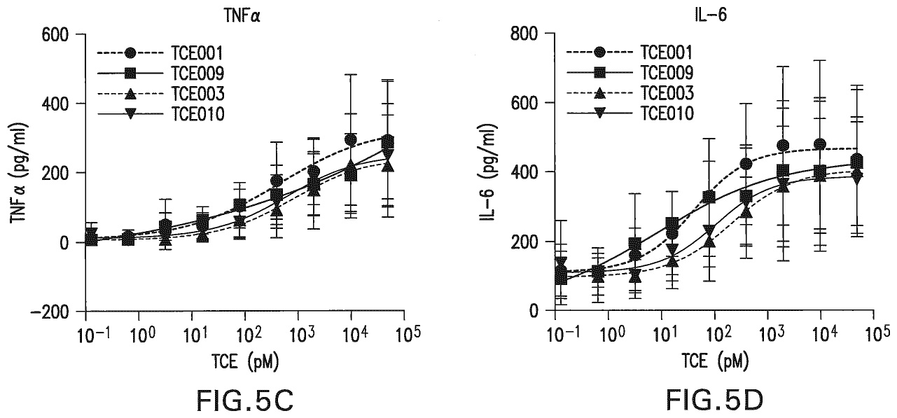

Following incubation, samples were pelleted by centrifugation, supernatant was discarded, and 200 μL FACs buffer was added to wash cells. Samples were again centrifuged to pellet cells, supernatant was discarded, and samples were resuspended in fixation buffer (eflioscience™ IC Fixation Buffer, ThermoFisher Scientific; Catalog No. 00-8222-49, used as a 2× solution, diluted at 1:2 in distilled water (ThermoFisher Scientific; Catalog No. 15230162)), followed by incubation for ten minutes in the dark at ambient temperature to fix cells. Samples were washed twice; each wash was performed by pelleting cells, discarding supernatant, and resuspending in 200 μL PBS. After the final wash, samples were pelleted by centrifugation and resuspended in 120 μL of PBS for flow cytometry analysis using a BD LSRFortessa cell analyzer. CD20+ B cell counts were obtained and normalized to untreated control wells to quantify percentage depletion of B cells in response to TCE (i.e. TCE001, TCE009, TCE003, TCEO10) treatment as reported in FIG. 5A.

Cytokine secretion in the supernatants was assessed by using a NHIP ProcartaPlex Mix & Match 6-plex kit (ThermoFisher; Catalog No. PPX-06-MXXGT3M). The manufacturer's recommended protocol for analyte detection was followed. Results were quantified using a Luminex xMAP INTELLIFLEX System (ThermoFisher; Catalog No. APX2020).

These results demonstrated that TCEs (i.e. TCE001, TCE009, TCE003, TCE010) effectively depleted targeted B cell populations in cyno PBMCs (FIG. 5A) with concentration-dependent induction of measured cytokines IFNγ (FIG. 5B), TNFα, (FIG. 5C) and IL-6 (FIG. 5D).

### Example 18: In Vivo B Cell Depletion in hCD19×hCD3Epsilon-Epitope Knock-In Mice Using CD19×CD3 TCEs

To investigate the in vivo efficacy of the TCE molecules, 0.1 mg/kg of TCE (TCE001, TCE009, TCE003; all with the RFHis modification as described in Example 9) was administered by subcutaneous injection to double knock-in mice expressing human CD19 and the binding epitope of human CD3 epsilon (hCD19×hCD3ε-epi). These genetically modified mice were generated by crossing Shanghai Model Organism huCD19 knock in mice on a C57BL/6J background (model NM-HTU-00110, Shanghai Model Organisms(Sugar Land TX)), in which the mouse CD19exons 1-4 were replaced by human CD19 exons 1-4,with human CD3e-epitope knock in mice generated as described in Zorn, Julie A., et al. Scientific Reports 12.1 (2022): 3530 (huCD3e:epi01)(B6N). Blood was collected from each animal 6 hours after TCE treatment and serum analytes were measured using a Luminex™ bead-based multiplexed assay. Blood, spleen and bone marrow (BM) were collected on day 3 post-TCE administration for flow cytometry analysis of B cell depletion.

For multicolor flow cytometry analysis, tissues were processed as follows: mouse spleens were collected and immediately placed in C tubes (Miltenyi Biotec; Catalog No. 130-093-237) containing 5 mL of ice-cold RPMI (ThermoFisher; Catalog No. 12633012). Single-cell dissociation was achieved using a GentleMACS Miltenyi dissociator instrument. Once the dissociator program was complete, samples were centrifuged for 5 minutes (Eppendorf 5810R centrifuge). The resulting pellet was resuspended in ammonium-chloride-potassium (ACK) buffer (VWR; Catalog No. 118-156-101) to remove red blood cells. The suspension was transferred to a 50 mL conical tube by passing it through a 70-micron filter, and 20 mL of RPMI was added. After an additional centrifugation (5 minutes at 1300 RPM), the pellet was resuspended in 5 mL of prepared media (RPMI containing 2% FBS (ThermoFisher; Catalog No. A5256701)) and was ready for flow cytometry antibody staining.

Whole blood and bone marrow were handled similarly. A volume (100 μL of blood (collected in Li-heparin tubes) and/or bone marrow (1 mL PBS flush of mouse femur) was transferred to a 96 deep-well plate (Eppendorf Corporation; Catalog No. 951033502). A volume (2 mL) of ACK buffer was added and incubated at room temperature for 10 minutes to remove red blood cells. The plate was centrifuged at 2200 RPM for ten minutes (Eppendorf 5810R centrifuge), and the resulting pellets were resuspended in DPBS buffer. The DPBS buffer wash was repeated once, and the cell pellets were resuspended in RPMI with 5% FBS (200 μL). The cells were transferred to a 96-well plate (ThermoFisher; Catalog No. 268152) and were ready for flow cytometry antibody staining.

The single-cell suspensions for blood, bone marrow, and spleen were transferred to a U-bottom 96-well plate (200 μL) and centrifuged at 1350 RPM for 5 minutes at 4° C. The resulting pellets were resuspended in DPBS buffer containing LIVE/Dead Near IR Dye (Life Technologies Live/Dead Fix NIR-400; Catalog No. L34976) and incubated for 15 minutes (on ice and in the dark). FACS buffer (100 μL) was added, and the plate was centrifuged as above. The cells were washed with FACS media and centrifuged again as described above. The cell pellets were resuspended in DPBS buffer containing 5% mouse serum for 10 minutes to block non-specific binding. An antibody mix (50 μL, see Table E23) was added and the solution was incubated for 30 minutes (on ice and in the dark). Once incubation was complete, 100 μL of FACS buffer was added, and the plate was centrifuged as described above. Cell pellets were then resuspended in 100 μL of fixation buffer (FoxP3 Staining Kit from ThermoFisher; Catalog No. 00-5523-00). Cells were then washed with 100 μL of permeabilization buffer provided in the staining kit. The plate was centrifuged as described above, and the supernatant was decanted into an appropriate receptacle. Pellets were resuspended in 200 μL of FACS buffer and were analyzed. Table E23 lists the panel of flow cytometry antibodies used in the in vivo hCD19×hCD3ε-epi double knock-in mouse study.

TABLE E23
Flow cytometry panel for evaluation of B cell depletion in
hCD19xhCD3e-epi double knock-in mice

| Fluorophore | Target | Clone | Dilution | Vendor | Catalog # |
|---|---|---|---|---|---|
| BUV496 | CD3 | 17A2 | 1:200 | BD | 569671 |
| BUV737 | CD4 | GK1.5 | 1:200 | BD | 612761 |
| BUV395 | CD45 | 30-F11 | 1:200 | BD | 564279 |
| BV711 | CD21/35 | 7.00E+09 | 1:200 | Biolegend | 123435 |
| BV605 | CD8a | 53-6.7 | 1:200 | Biolegend | 100744 |
| BV510 | NKp46 | 29A1.4 | 1:100 | Biolegend | 137623 |
| BV421 | CD24 | M1/69 | 1:100 | Biolegend | 101826 |
| PE | CD20 | SA275G2 | 1:100 | Biolegend | 152106 |
| AF488 | huCD19 | HIB19 | 1:100 | Biolegend | 302219 |
| PE-Cy7 | B220 | RA3-6B2 | 1:200 | Biolegend | 103222 |
| eFluor780 | Live/Dead | N/A | 1:1000 | ThermoFisher | 65-0865-14 |
| AlexaFluor647 | CD11B | M1/70 | 1:200 | Biolegend | 101218 |

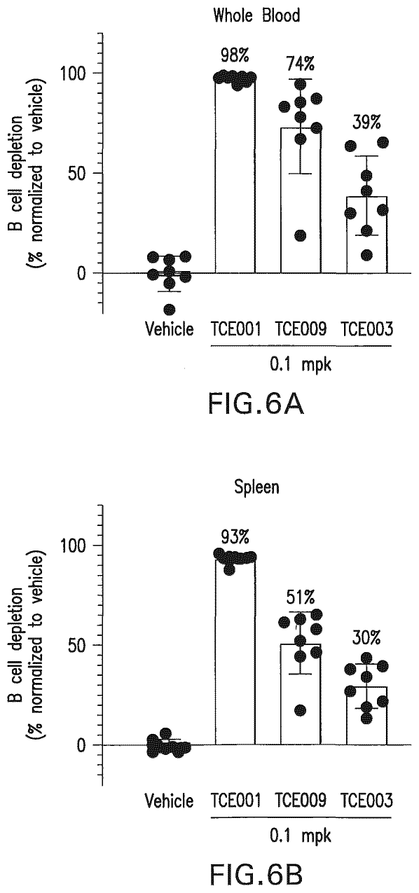

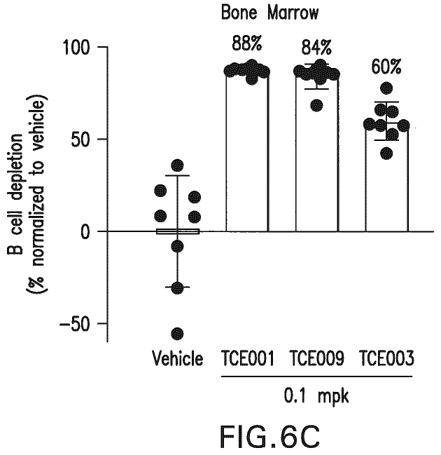

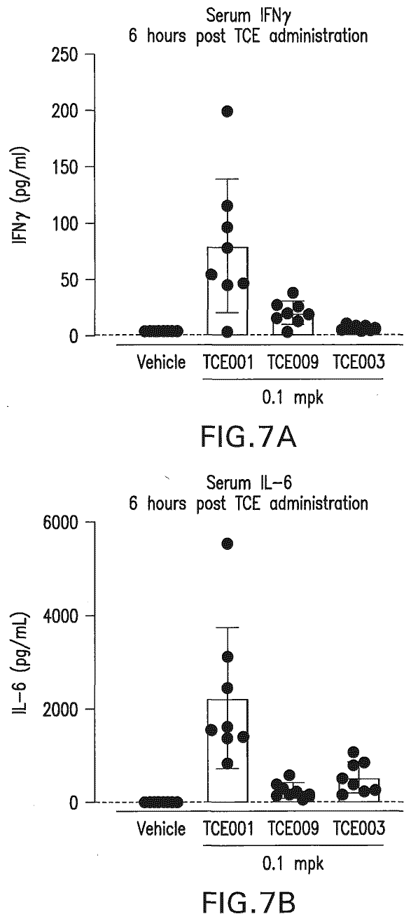

B cells were identified as CD45+ B220+ cells. The percentage depletion of B220+ B cells by TCE001, TCE009 and TCE003 was determined relative to the vehicle control, as depicted in FIG. 6. Profound B cell depletion in response to TCE001 was observed in the whole blood (FIG. 6A; TCE001_{mean % depletion}=98%), spleen (FIG. 6B; TCE001_{mean % depletion}=93%), and bone marrow (FIG. 6C; TCE001_{mean % depletion}=88%). To investigate cytokine induction after TCE treatment, blood was collected at 6 hours post TCE administration. Induction of serum IFNγ (FIG. 7A) and serum IL-6 (FIG. 7B) was observed in response to TCE administration (e.g., TCE001).

Together, these data conclusively demonstrated that TCE001 effectively mediated substantial B cell depletion in vivo within a mouse model, highlighting the strong therapeutic potential of TCE001.

### Example 19: An In Vitro PBMC Depletion Assay Using the CD19×CD3 TCE001

This example compares the B cell depletion efficiency and cytokine induction levels in an in vitro assay between three CD19×CD3 TCEs: (i) TCE001, (ii) blinatumomab biosimilar (referred to herein as “blinatumomab bs”; purchased from Proteogenix; Catalog No. PX-TA1067), and (iii) tAb0032 (SEQ ID NO: 241), the latter of which is a bispecific binding protein disclosed in WO 2019/237081 which contains (i) an anti-CD19 scFv (having the amino acid sequence set forth in SEQ ID NO: 242), (ii) an anti-CD3 scFv (having the amino acid sequence set forth in SEQ ID NO: 243), and (iii) an anti-HSA sdAb (having the amino acid sequence set forth in SEQ ID NO: 244).

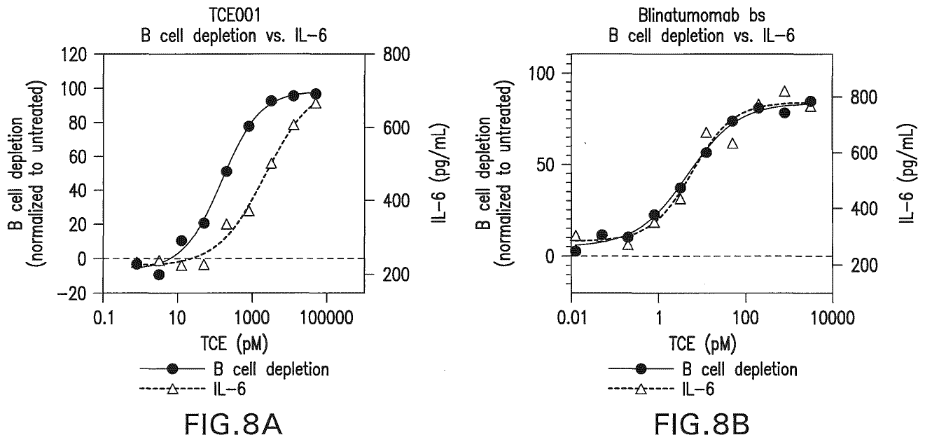

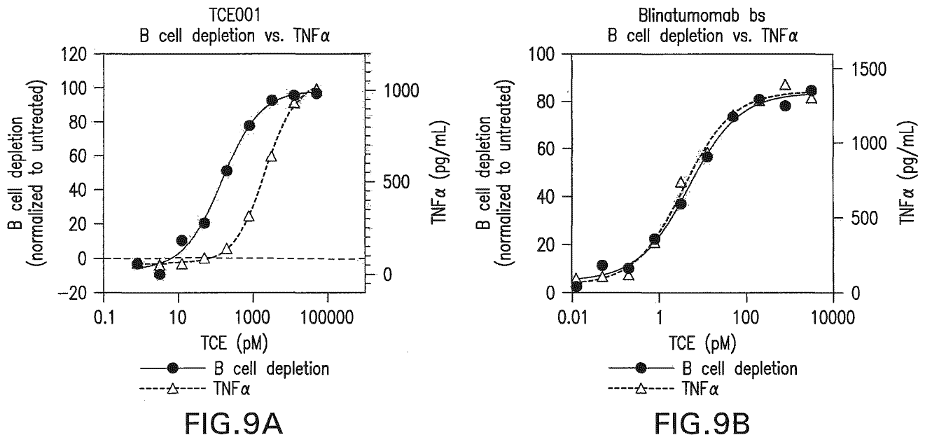

Briefly, human PBMCs were exposed to increasing concentrations of TCE001, blinatumomab bs, or tAb0032 as described above in the human PBMC depletion assay (see Example 15). Following incubation, flow cytometry analysis of B cell depletion and analysis of cytokine induction were conducted according to the methodologies described in Example 15. Concentration-response data were analyzed using nonlinear regression curve fitting. Comparative plots of B cell depletion versus IL-6 secretion for TCE001 and blinatumomab bs are shown in FIGS. 8A and 8B, and B cell depletion versus TNFα secretion for TCE001 and blinatumomab bs are shown in FIGS. 9A-9B. EC₅₀ values for B cell depletion and cytokine release are presented in Tables E24, E25, E26. For each molecule, the fold change difference between the EC₅₀ for cytokine release and the EC₅₀ for B cell depletion was calculated (Tables E24, E25, E26).

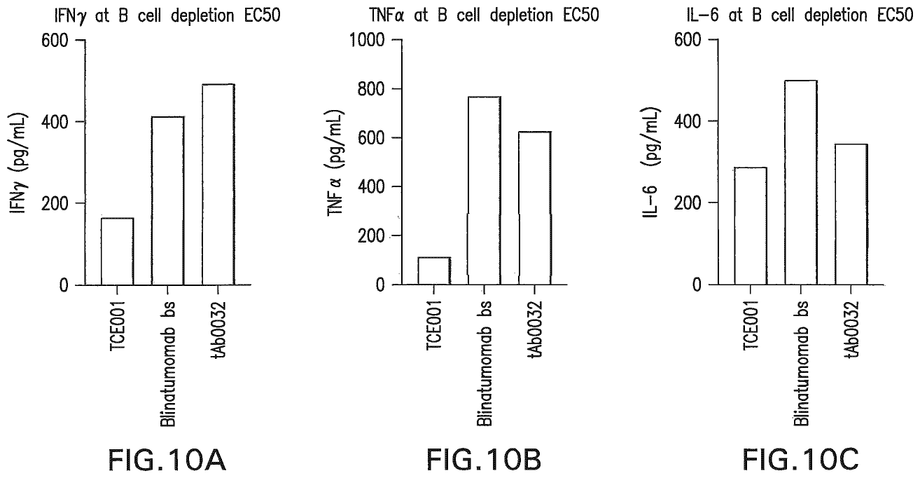

The results showed that TCE001 demonstrated a superior therapeutic window between B cell depletion and cytokine induction relative to blinatumomab bs or tAb0032, as evidenced by the enhanced separation between B cell depletion and cytokine release concentration response curves as depicted in FIGS. 8A, 8B, 9A and 9B, as well as the reduced cytokine secretion at either B cell depletion EC₅₀ concentration or B cell depletion EC₉₀ concentration depicted in FIGS. 10A-10C (see corresponding data presented in Table E27 for B cell depletion EC₅₀ concentration and Table E28 for B cell depletion EC₉₀ concentration). For example, TCE001 demonstrated a wider therapeutic window compared to blinatumomab bs and tAb0032 as reflected by the larger fold differences in EC₅₀ for B cell depletion and IFNγ, TNFα and IL-6 release of 6-fold, 14-fold and 14-fold, respectively (Table E24). In contrast, the lower fold differences observed with blinatumomab bs and tAb0032 for the same tested cytokines (mostly between 0.74 and 3.5; see Tables E25 and E26) indicated there is little separation between the dosing required for cell depletion and cytokine induction. This narrow margin appears to suggest a heightened risk of immune-related adverse events at therapeutically relevant doses for blinatumomab bs and tAb0032. Importantly, and consistent with the inventors' hypothesis, at comparable depletion levels, TCE001 also induces lower cytokine levels than blinatumomab bs or tAb0032 for all cytokines tested (Table E27, Table E28, FIGS. 10A-10C), demonstrating that TCE001 can achieve comparable depth of B cell depletion as blinatumomab bs and tAb0032, but at a substantially lower risk of cytokine release.

TABLE E24
Potency of B cell depletion and cytokine release
in response to TCE001

| | B cell | | | |
|---|---|---|---|---|
| | depletion | IFNγ | TNFα | IL-6 |
| EC₅₀ (pM) | 152 | 973 | 2159 | 2138 |
| Fold change | | 6 | 14 | 14 |
| relative to B | | | | |
| cell depletion | | | | |
| EC₅₀ | | | | |

TABLE E25
Potency of B cell depletion and cytokine release in
response to blinatumomab bs

| | B cell | | | |
|---|---|---|---|---|
| | depletion | IFNγ | TNFα | IL-6 |
| EC₅₀ (pM) | 4.7 | 4.8 | 3.5 | 6 |
| Fold change | | 1.0 | 0.74 | 1.3 |
| relative to B | | | | |
| cell depletion | | | | |
| EC₅₀ | | | | |

TABLE E26
Potency of B cell depletion and cytokine
release in response to tAb0032

| | B cell | | | |
|---|---|---|---|---|
| | depletion | IFNγ | TNFα | IL-6 |
| EC₅₀ (pM) | 0.2 | 0.7 | 0.3 | 1.5 |
| Fold change | | 3.5 | 1.5 | 7.5 |
| relative to B | | | | |
| cell depletion | | | | |
| EC₅₀ | | | | |

TABLE E27
Cytokine levels at equivalent B cell depletion potency
(B cell depletion EC₅₀)

| | TCE001 | Blinatumomab bs | tAb0032 |
|---|---|---|---|
| IFNγ (pg/mL) | 167 | 415 | 495 |
| TNFα (pg/mL) | 116 | 772 | 629 |
| IL-6 (pg/mL) | 290 | 503 | 347 |

TABLE E28
Cytokine levels at equivalent B cell depletion potency
(B cell depletion EC₉₀)

| | TCE001 | Blinatumomab bs | tAb0032 |
|---|---|---|---|
| IFNγ (pg/mL) | 504 | 617 | 922 |
| TNFα (pg/mL) | 540 | 1252 | 1243 |
| IL-6 (pg/mL) | 464 | 744 | 624 |

Therefore, these data demonstrated TCE001's superior therapeutic window between B cell depletion and cytokine induction, suggesting a safer, more tolerable, and clinically desirable profile compared to blinatumomab bs and tAb0032.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

TABLE E29
WITH AMINO ACID SEQUENCES AND NUCLEIC ACID
SEQUENCES

| SEQ | | |
|---|---|---|
| ID | | |
| NO: | Sequence | Description |
| 1 | MPPPRLLFFLLFLTPMEVRPEEPLVVKVEEGDNAVLQCLKGTS | H. sapiens |
| | DGPTQQLTWSRESPLKPFLKLSLGLPGLGIHMRPLAIWLFIFNV | CD19 |
| | SQQMGGFYLCQPGPPSEKAWQPGWTVNVEGSGELFRWNVSD | amino acid |
| | LGGLGCGLKNRSSEGPSSPSGKLMSPKLYVWAKDRPEIWEGEP | sequence |
| | PCLPPRDSLNQSLSQDLTMAPGSTLWLSCGVPPDSVSRGPLSW | GenBank: P15391 |
| | THVHPKGPKSLLSLELKDDRPARDMWVMETGLLLPRATAQDA | |
| | GKYYCHRGNLTMSFHLEITARPVLWHWLLRTGGWKVSAVTL | |
| | AYLIFCLCSLVGILHLQRALVLRRKRKRMTDPTRRFFKVTPPPG | |
| | SGPQNQYGNVLSLPTPTSGLGRAQRWAAGLGGTAPSYGNPSS | |
| | DVQADGALGSRSPPGVGPEEEEGEGYEEPDSEEDSEFYENDSN | |
| | LGQDQLSQDGSGYENPEDEPLGPEDEDSFSNAESYENEDEELT | |
| | QPVARTMDFLSPHGSAWDPSREATSLGSQSYEDMRGILYAAPQ | |
| | LRSIRGQPGPNHEEDADSYENMDNPDGPDPAWGGGGRMGTW | |
| | STR | |
| 2 | AGGCCCCTGCCTGCCCCAGCATCCCCTGCGCGAAGCTGGGT | H. sapiens |
| | GCCCCGGAGAGTCTGACCACCATGCCACCTCCTCGCCTCCTC | CD19 |
| | TTCTTCCTCCTCTTCCTCACCCCCATGGAAGTCAGGCCCGAG | nucleotide |
| | GAACCTCTAGTGGTGAAGGTGGAAGGTATGTCCAAAGGGCA | sequence |
| | GAAAGGGAAGGGATTGAGGCTGGAAACTTGAGTTGTGGCTG | GenBank: |
| | GGTGTCCTTGGCTGAGTAACTTACCCTCTCTGAGCCTCCATT | NG_007275.1 |
| | TTCTTATTTGTAAAATTCAGGAAAGGGTTGGAAGGACTCTG | |
| | CCGGCTCCTCCACTCCCAGCTTTTGGAGTCCTCTGCTCTATA | |
| | ACCTGGTGTGAGGAGTCGGGGGGCTTGGAGGTCCCCCCCAC | |
| | CCATGCCCACACCTCTCTCCCTCTCTCTCCACAGAGGGAGAT | |
| | AACGCTGTGCTGCAGTGCCTCAAGGGGACCTCAGATGGCCC | |
| | CACTCAGCAGCTGACCTGGTCTCGGGAGTCCCCGCTTAAAC | |
| | CCTTCTTAAAACTCAGCCTGGGGCTGCCAGGCCTGGGAATC | |
| | CACATGAGGCCCCTGGCCATCTGGCTTTTCATCTTCAACGTC | |
| | TCTCAACAGATGGGGGGCTTCTACCTGTGCCAGCCGGGGCC | |
| | CCCCTCTGAGAAGGCCTGGCAGCCTGGCTGGACAGTCAATG | |
| | TGGAGGGCAGCGGTGAGGGCCGGGCTGGGGCAGGGGCAGG | |
| | AGGAGAGAAGGGAGGCCACCATGGACAGAAGAGGTCCGCG | |
| | GCCACAATGGAGCTGGAGAGAGGGGCTGGAGGGATTGAGG | |
| | GCGAAACTCGGAGCTAGGTGGGCAGACTCCTGGGGCTTCGT | |
| | GGCTTCAGTATGAGCTGCTTCCTGTCCCTCTACCTCTCACTG | |
| | TCTTCTCTCTCTCTGCGGGTCTTTGTCTCTATTTATCTCTGTC | |
| | TTTGAGTCTCTATCTCTCTCCCTCTCCTGGGTGTCTCTGCATT | |
| | TGGTTCTGGGTCTCTTCCCAGGGGAGCTGTTCCGGTGGAATG | |
| | TTTCGGACCTAGGTGGCCTGGGCTGTGGCCTGAAGAACAGG | |
| | TCCTCAGAGGGCCCCAGCTCCCCTTCCGGGAAGCTCATGAG | |
| | CCCCAAGCTGTATGTGTGGGCCAAAGACCGCCCTGAGATCT | |
| | GGGAGGGAGAGCCTCCGTGTCTCCCACCGAGGGACAGCCTG | |
| | AACCAGAGCCTCAGCCAGGGTATGGTGATGACTGGGGAGAT | |
| | GCCGGGAAGCGGGGGTCCAGAGACAGAGGGGAGGGGAAAC | |
| | TGAAGAGGTGAAACCCTGAGGATCAGGCTTTCCTTGTCTTAT | |
| | CTCTCCCTGTCCCAGACCTCACCATGGCCCCTGGCTCCACAC | |
| | TCTGGCTGTCCTGTGGGGTACCCCCTGACTCTGTGTCCAGGG | |
| | GCCCCCTCTCCTGGACCCATGTGCACCCCAAGGGGCCTAAG | |
| | TCATTGCTGAGCCTAGAGCTGAAGGACGATCGCCCGGCCAG | |
| | AGATATGTGGGTAATGGAGACGGGTCTGTTGTTGCCCCGGG | |
| | CCACAGCTCAAGACGCTGGAAAGTATTATTGTCACCGTGGC | |
| | AACCTGACCATGTCATTCCACCTGGAGATCACTGCTCGGCC | |
| | AGGTAGAGTTTCTCTCAACTGGGAGGCATCTGTGTGGGGGT | |
| | ACTGGGAAGAAGTGGAAGCCAGTCAATCTTAGATTCCCCCA | |
| | ACCCGAGGGCTACTCCCAGCCTCACCCCAAACCCCAACTTC | |
| | CACACAGAACACTGACTCCAAGTCTTTCTTTTTTTTGACAGA | |
| | GTCTCGCTCTGTTGCCTAGGCTGGAGTGCAGTGGTGCCATCT | |
| | TGTCTTGGCTCACTGCAACCTCCGCCTCCCAGGTTCAAGTGA | |
| | TTCCCCTGCCTCAGCCTCCTGAGTAGCTGGGATTACAGGTGC | |
| | CCACCACCACGCCTGGCTAATTTTTTTTTTTTTTTTGAGACGG | |
| | AGTCTTGCACTGTCACCCAGGCTGGAGTGCAGTGGCACGAT | |
| | CTCAGCTCACTGCAACCTCCACCTTCCAGGTTCAAGTGATTC | |
| | TCCTGCCTCAGCCTCCCGAGTAGCTGGGATTAAAGCCTGGCT | |
| | AATTTTTTTTGTATTTTTAGTAGAGATGGGGTTTCATTATGTT | |
| | GGCCAGGCTGGTCTCAAACTCCTGACCTCGTGATCCACCCG | |
| | CCTCGGCCTCCCAAAGTGCTGGGATTACAGACATGAGCCAC | |
| | AGGGCCGGGCCAAGCCTAATTTTGTATTTTTAGTAGAGATG | |
| | GGGTTTCTCCCTGTTGGACCAGGCTGGTCTTGAACTCCTGAC | |
| | TTCAGGTGATCTGCCTGCCTTGGCCTCCCAAAGTACTGGGAT | |
| | TACAGGCATAAGCCACCGCACCTGGCCTAGACTTCAAGTCT | |
| | TTCTTCCCTCGCTTCCAAGACACTACTTTTCTGGGTCTTCACC | |
| | TACCATTGCTTGCGCCTGCCCACCAGCTTGGGTGGAGTCTTC | |
| | CTTCCTCCCCAACTCCTCACTCTTGGAGCCCTGGGCCCTCTT | |
| | CTTATCCCTGTCTGCACACTTTCCTATTTGAACTTGACTCTCA | |
| | ATGGCTTCTTGGGTCACCATGCCTTGGTGACTCTATTCCAGG | |
| | CTCCATACTCAGCCATCTCCTGTGCCATTTGATATCCCATGG | |
| | ACACCTCAGGCTCAACAGATACAAAATCAAACTCAATGTCT | |
| | TCCCCAAGTATAGTCTTCTTGGTGGCCCAGTGTAAGCAGAG | |
| | GGCACCACCACCTGCTCCCTCGCCCAGGCTAAGAACCTGGG | |
| | CATCCTTCTTTTTCCTCACCCCGTCCAACAAACTGGTCACAG | |
| | TGTTCTGCCAATTCTCTCTCCATGCAATCCTATCATGCTATCC | |
| | TAACTGCAATTCACAAACCCAACCCCAACTTTCACTCCAAA | |
| | CTTGATCCAAGCAATGTGCTGGATCCCAACTGTAACCTTGCA | |
| | AACTCAACTCTGCCCTTCACTTTGACCGTGACTATCCTTAAT | |
| | TGCAGCAGGAAACTGATCATTATGCTCCCCTCAATCCACAC | |
| | ATTGCCTCTGAGTACAGCCATGGTTTGTCCACGATTTGCTCA | |
| | AAGACACTGCCCCATGTCCTGTGCCAGGGTCTGTGACAATC | |
| | CCTGACCTCCTGGGACATGGCTCCTTAGAGAGAGGAGAGCC | |
| | TTTCTCACAGCTTGGGACTTTGAGTCTGTGTCTTTTTTTTTTT | |
| | CTTGAGACGGAGTTTTGCTGTGGTTGCCCAGGCTGGAGTGC | |
| | AGTGATCTCGGCTCACTGAAACCTCCGCCTCCCGGGTTCAA | |
| | ACGATTCTCCTGCCTCAGCCTCCCAAGTAGCTGGGATTACAG | |
| | GCACCCACCACCATGCCCAGCTAATTTTTTTGTATTTTTAGT | |
| | AGAGATGGGGTTTCACCATGTTGGCCAGGCTGGTCTCGAAC | |
| | TCCTGACCTCAGGTGATCCACCCGCCTTTGCCTCCCAAAGTG | |
| | CTGGGATTACAGGCGTCAACCACCGCGCCCGGCCGAGTCTG | |
| | TGTCTTGCCTCTGTGCCTCAGACTTGCGGTTCCTTGAGATCT | |
| | CAGGATTGGGACGTAAGATGCCAGCCTGGGGTCCTCGTCTC | |
| | ATAGCCCCTTCCCCCTAGTACTATGGCACTGGCTGCTGAGGA | |
| | CTGGTGGCTGGAAGGTCTCAGCTGTGACTTTGGCTTATCTGA | |
| | TCTTCTGCCTGTGTTCCCTTGTGGGCATTCTTCATCTTCAAAG | |
| | AGGTGAGTCATGTCCCCAGTGGGTCTGTCCAAACCCTACTCC | |
| | ATCTTCCCCAGGATAAGCCGGCTCTGGCCAGTCTGACAACC | |
| | ATCTTTCTTTCCTCCCATCCCTCCCTTCAAGACCCCAGAATC | |
| | CTGTTCTCCCCAGTCTTCCTCTAGCCTCCCTCAAACTTCCCA | |
| | AGCCTCTTGCAATTTTTTTTTTTTTTTTGAGACAGGGTCTCAT | |
| | TCTGTCACCCCAGCTGGAGTGCAGTGGCACAATCTGAGCTC | |
| | ACTGTAACCTCTGCCTCCCAGGCTTAAGTGATTCTTGTGCTT | |
| | CAGCCTCCCGAGTACCTGGGACTACAAGTGTATGCCACCAC | |
| | ACCCGGCCAATTTTTTATATTTTTAGTAGAGACGAGGTTTCA | |
| | CCATGTTGGCCAGACTGGTCTCGAACTCTTGACCTCAAATGA | |
| | TCCGCCCACCTCGGCCTCCCAAAGTGCTGGGATTACAGGCA | |
| | CGAGCCACCGCGCCCGTCCGCCTCGCAATTTGAACTCCTGTC | |
| | TCCTTTGTTGAACCAAGTGACCTCCCCAGCACCTGGCCCCAC | |
| | AAATCCTCACCCTGCCAAGCAGCCCCTCCTCTGATCACGCCC | |
| | TTTAACTCCCACCAGCCCTGGTCCTGAGGAGGAAAAGAAAG | |
| | CGAATGACTGACCCCACCAGGAGGTAATGCAACCAGTGCAC | |
| | CCCGCGGTAACACCCTCCACCTTCACTTTATGCCTTGCACTT | |
| | ACTGTTTCCTCTGCCCAGGGGTTCTTTGCTCCGTCTCTACTGT | |
| | TTCAAATACTGCCCAACCTCAAAGCCCAGCTCCAAAGCTAC | |
| | CTCCTCTGTGAAGAACTCCTTGGAAATGATCATCTCAGACTC | |
| | CTCTATTGGCTGTCCCAGCACAAGTGATCACGTTTAACTTCT | |
| | GAAGGCCTGGACAGAATCTTGAGTGGGTCCGCCATTCCATT | |
| | CCAAGTCGGCCCTCACCGTGCACTTCCTCTTCTCCCGCCAGA | |
| | TTCTTCAAAGTGACGCCTCCCCCAGGAAGCGGGCCCCAGAA | |
| | CCAGTACGGGAACGTGCTGTCTCTCCCCACACCCACCTCAG | |
| | GCCTCGGTAAGAGGCACCGCCCCTCCAGCCTATAGCTCCGC | |
| | CCCAGATCCGGGGCTCCACCCCCACTCTCCTCATCCCTCCAA | |
| | TCCGCTGTGCGCCAAGCCTTCTGGAGCTCGGAACTCCGCCCC | |
| | CGGGGCGGGGAGTCCCGCCCAGCTATGAGCCCCGCCTCTAG | |
| | AACCAGACCCCGCCTCCAGGGCTCAGAGCCACGCCCCCAGG | |
| | ACCCAGAGCCTGAAGTCGTAATCAAGAGCAGAACTTCGCCC | |
| | CAGAACTGAAGGCCTCGGCCCTAGATTTAGATTCCGCCCCA | |
| | GGGTTCAAGGCCGGGTTCCTAGACCCAGAGTCCATTCGCAG | |
| | AGCCCAAAACATCCTCTTCCCGTGCCCCGCCGCGCGGACCC | |
| | TTAGCCTTGACCGCCCCCATCTCTTCTGACCCCGTCTTACAA | |
| | TGCCCCTCTCACCAGGACGCGCCCAGCGTTGGGCCGCAGGC | |
| | CTGGGGGGCACTGCCCCGTCTTATGGAAACCCGAGCAGCGA | |
| | CGTCCAGGCGGATGGAGCCTTGGGGTCCCGGAGCCCGCCGG | |
| | GAGTGGGTGAATGACTGGGAGAGGGAAGGGTCGTTCCCCAC | |
| | ATGGAGGGGGTTGGAGCGGTCTGTGGCCCGAATAGTGGACT | |
| | GGGCCCTGGAGGAGAGGGGGCATGACTCGGTTCCCCATCCC | |
| | CATCCCCAAACCCCCAGGCCCAGAAGAAGAGGAAGGGGAG | |
| | GGCTATGAGGAACCTGACAGTGAGGAGGACTCCGAGTTCTA | |
| | TGAGAACGACTCCAACCTTGGGCAGGACCAGCTCTCCCAGG | |
| | GTAAGGCTGCCCTCCCCCGTGGCCCCCCACCTCTGCGGTGGC | |
| | CTGTGGACTCCCATGGACACCCCTCCTTCTCCACCAGATGGC | |
| | AGCGGCTACGAGAACCCTGAGGATGAGCCCCTGGGTCCTGA | |
| | GGATGAAGACTCCTTCTCCAACGGTAACTTGGGGCCTTTGTG | |
| | GGACCTCAGAGACTTAGGTGTAATTGCAGCGCTGTGACACT | |
| | CCTAGAAGGGGTCCCTGGAGTTCTCTCTCTTCTGCCACAGCT | |
| | GAGTCTTATGAGAACGAGGATGAAGAGCTGACCCAGCCGGT | |
| | CGCCAGGACAATGGGTGTGTGTGAGGATGGCAACAGTCCAG | |
| | GGGGGAGGCGGAGGACACCTGGAGGCCAGGAGGAATAGTA | |
| | ACCTCCCTCTTCCCTTTCCAGACTTCCTGAGCCCTCATGGGT | |
| | CAGCCTGGGACCCCAGCCGGGAAGCAACCTCCCTGGGTGAG | |
| | AGATGCTTTCAATCAGACTGCCTTGCCCAGCTTGGGTGACCT | |
| | GGCCTCAGCTCTGACACCAGATCCAACTTTGACCTGACCCTG | |
| | ACCCCAAACCCGAACCCAATCCTGTGACTCCTCTCACCTCAA | |
| | CACTGAGCCCCATCCCCCATCCTGAGCCCCATCCCCCATCCT | |
| | GACCCCCAATATTTACCCCCTCCCTAACTGTGAATATCAACA | |
| | CCGATCCCAATGCAGTATCAGCCTGGACTTGATCTCCACCTC | |
| | ACCTCAGCCCCAGTGCAGACCTCAACTTGGACCCCAGCTTA | |
| | CTCTGCAGCTTCTTCATGACTCTGACTCCGACTCCCTCCAGT | |
| | TTCTTCTTTTTCTTTTTCTTTTTTTTGAGACGGAGTCTCCCTCT | |
| | GTTGCCCAGGCTGGAGTGCAGTTGCCACCTCTGCCTCCTAGG | |
| | TTCAAGCGATTCTCATGCCTCAGCCTCCTGAGTAGCTGGGAT | |
| | TATAGACGTTTGCCACCACACCTGGCTAATTTTTGTATTTTC | |
| | AGTAGAGACAGGGTTTCGCCATGTTGGCCAGACTGGTCTCC | |
| | AACTCCTGGCCTCTAGTGATCTGCCCGCCTTGGCTTCCCAAA | |
| | GTGCTGGGATTACAGGCATGAGCCACCACGCCCAGCCCAGT | |
| | TCTGTTCTTGACCCCTTCCTTAGCCATAATCTAACCCATATCT | |
| | AACCCTGACCCTACAGCTAACTGGGGCCCCAAACTCAATGC | |
| | TAACCAAATCACCCCTTCCCAGCACAGCATGGGTAATGCTC | |
| | CTCACCTTCCTCTGCCCCTCAGTCTTCCTCCTTACCGTAGGCT | |
| | GTACTTCCCATGCCCTAGCCTCCAATTCTCCATCCCCCGCCC | |
| | AAGCAGGGTCCCAGTCCTATGAGGATATGAGAGGAATCCTG | |
| | TATGCAGCCCCCCAGCTCCGCTCCATTCGGGGCCAGCCTGG | |
| | ACCCAATCATGAGGAAGGTGGGTGCTTCTGCCGCTGTCCCC | |
| | TGCTGTCCCCTGGGCTGACTTTGCCTTCCAGCCTACTTCCAG | |
| | TGCCACCCATGTTCTCCTCCTCCCTGGTCCTATCCAGATGCA | |
| | GACTCTTATGAGAACATGGATAATCCCGATGGGCCAGACCC | |
| | AGCCTGGGGAGGAGGGGGCCGCATGGGCACCTGGAGCACC | |
| | AGGTGATCCTCAGGTGGCCAGGTGAGCTGGGACTGCCCCTA | |
| | GGGAAAGCGGGGAGGGAGGGAGATAGGCACGGATGGCAGT | |
| | GGCTGCTGGCTTTCAGGGAGGGAGAGGGAACAGGGTTCCTA | |
| | GGGCCTGGTGGGCAGGGGGAGGACTGCTGGACCCCTCCCCA | |
| | TCACCGTTTCTTCTGCATAGCCTGGATCTCCTCAAGTCCCCA | |
| | AGATTCACACCTGACTCTGAAATCTGAAGACCTCGAGCAGA | |
| | TGATGCCAACCTCTGGAGCAATGTTGCTTAGGATGTGTGCAT | |
| | GTGTGTAAGTGTGTGTGTGTGTGTGTGTGTGTATACATGCCA | |
| | GTGACACTTCCAGTCCCCTTTGTATTCCTTAAATAAACTCAA | |
| | TGAGCTCTTCCAATCCTA | |
| 3 | MPPPCLLFFLLFLTPMEVRPQEPLVVKVEEGDNAVLQCLEGTS | Macaca |
| | DGPTQQLVWCRDSPFEPFLNLSLGLPGMGIRMGPLGIWLLIFNV | fascicularis |
| | SNQTGGFYLCQPGLPSEKAWQPGWTVSVEGSGELFRWNVSDL | CD19 |
| | GGLGCGLKNRSSEGPSSPSGKLNSSQLYVWAKDRPEMWEGEP | amino acid |
| | VCGPPRDSLNQSLSQDLTMAPGSTLWLSCGVPPDSVSRGPLSW | sequence |
| | THVRPKGPKSSLLSLELKDDRPDRDMWVVDTGLLLTRATAQD | GenBank: F7F486 |
| | AGKYYCHRGNWTKSFYLEITARPALWHWLLRIGGWKVPAVT | |
| | LTYLIFCLCSLVGILQLQRALVLRRKRKRMTDPTRRFFKVTPPP | |
| | GSGPQNQYGNVLSLPTPTSGLGRAQRWAAGLGGTAPSYGNPS | |
| | SDVQVDGAVGSRSPPGAGPEEEEGEGYEEPDSEEGSEFYENDS | |
| | NFGQDQLSQDGSGYENPEDEPLGPEDEDSFSNAESYENEDEEL | |
| | TQPVARTMDFLSPHGSAWDPSREATSLGSQSYEDMRGLLYAA | |
| | PQLRTIRGQPGPNHEEDADSYENMDNPDGPDPAWGGGGRMG | |
| | TWSAR | |
| 4 | GGGTGCCCCGGGGAGTCTGACCACCATGCCACCTCCTTGCC | Macaca |
| | TCCTCTTCTTCCTCCTCTTCCTCACCCCCATGGAAGTCAGGC | fascicularis |
| | CCCAGGAACCTCTAGTGGTGAAGGTAGAAGGTATGTCCAAA | CD19 |
| | GGGCAGAAAGGGAAGGGATGGAGGCTGGAAACTTGGGTTG | nucleotide |
| | TGGCTGGGTGTCCTTGGCTGAGTAACTTACCCTCTCTGAGCC | sequence |
| | TCCATTTTCTTATTTGTGAAATTCAGGGAGGGGTTGGAAGGA | GenBank: |
| | CTCTGCCGGCTCCTCCACTCCCAGTTTTTGGAGTCCTCTGAT | NC_088394.1 |
| | CTATAACCCGGTGTGAGCAGTGGGGGGGCTTGGAGGTCCCC | |
| | CCGTCATGCCCACACCTCTCTCCCTCTCTCTCCACAGAGGGA | |
| | GATAACGCTGTGCTGCAGTGCCTCGAGGGGACCTCAGATGG | |
| | CCCCACTCAGCAGCTGGTCTGGTGTCGGGACTCCCCGTTTGA | |
| | ACCCTTCTTAAATCTCAGCCTGGGGCTGCCAGGCATGGGAA | |
| | TCCGCATGGGGCCCCTGGGCATCTGGCTTTTAATCTTCAACG | |
| | TCTCTAACCAGACGGGGGGCTTCTACCTATGCCAGCCGGGG | |
| | CTCCCCTCTGAGAAGGCCTGGCAGCCTGGCTGGACAGTCAG | |
| | TGTGGAGGGCAGCGGTGAGGGCCGGGCTGGCCGGGCTAGG | |
| | GCAGGGGCAGGAGGAGAGAAGGGAGGCCACCATGGACAGA | |
| | AGAAGTCCGCGGCCACAATGGAGCTGGAGAGAGGGGCTGG | |
| | AGGGATTGAGGGCGAAACTCGGAGCTAGGTGGGCAGACTC | |
| | CTGGGGCTTCGTGGCTTCAGCATGAGCTGCTTCCTGTCCCTC | |
| | CACCTCTCACTGCCTTCTCTCTCTGTCTCTCTCTCTCTGAGTC | |
| | TTTGTCTCTATTTATCTCTGCCTTTGAGTCTCTCTCTCTCTCC | |
| | CTCTCCTGGGTGTCTCTGCATTTGGTTCTGGGTCTCTTCCCAG | |
| | GGGAGCTGTTCCGGTGGAATGTTTCGGACCTAGGTGGCCTG | |
| | GGCTGTGGCCTGAAGAACAGGTCCTCAGAGGGCCCCAGCTC | |
| | CCCTTCTGGGAAACTCAACAGCTCCCAACTGTATGTGTGGG | |
| | CCAAAGACAGACCTGAGATGTGGGAGGGAGAGCCTGTGTGT | |
| | GGCCCACCGAGGGACAGCCTGAACCAGAGCCTGAGCCAGG | |
| | GTAAGGTGATGATTGGGGAGATGCCAGGAAGTGGGGGTCC | |
| | AGAGACGGAGGGGAGGGGAAACTGAAGAGGTGTGAGACCC | |
| | TGAGGATCAGGCTTTCCTCGTCTTATCTCTCCCTGCCCCAGA | |
| | CCTCACCATGGCCCCGGGCTCCACACTCTGGCTGTCCTGTGG | |
| | GGTACCCCCTGACTCTGTGTCCAGGGGCCCCCTCTCCTGGAC | |
| | CCATGTGCGCCCCAAGGGGCCTAAGTCCTCATTGTTGAGCCT | |
| | AGAGCTGAAGGACGATCGCCCAGACAGAGATATGTGGGTA | |
| | GTGGACACGGGTCTGTTGTTGACCCGGGCCACAGCTCAAGA | |
| | CGCTGGGAAGTATTATTGTCACCGTGGCAACTGGACCAAGT | |
| | CATTCTACCTGGAGATCACTGCTCGGCCAGGTAGAGTTTCTC | |
| | TCAACTGGGAGGCATCTGTGTGGGGTGTACTGGCAAGAAGT | |
| | GAAAGCCAGTCAATCTTAGGTTCCCCCAACCTGAGGGCTAC | |
| | TCCCAGCCTCACCCCAAACCCCAACTTCCACACAGAACATT | |
| | GACTCCAAGTCTTTTTTTTTTTTTTTGACCGAGTCTTGCTCTG | |
| | TCGCCTAGGCTGGAGTGCAGTGGTGCCATCTCGTCTTGGCTC | |
| | ACTGCAACCTCCGCCTCCCAGGTTCAAGCGATTCTCCTGCCT | |
| | CAGCCTCCTGAATAGCTGGGATTACAGGTGCCCACCACCAT | |
| | ACCTGGCTAATTTTTTTTTTTTGAGAGGAGTCTTTCACTGTCA | |
| | CCCAGGCTGGAGTGCAATGGTGCAATCTCAGCTCACTGCAA | |
| | CTTTCGCCTTCCAAGTTCAAGTGATTCTCCTGCCTCAGCCTC | |
| | CCGAGTAGCTGGGATTACAGGCGCTCGCCACCATGCCTGGC | |
| | TAATTTTTTTGTTTTTTTTTTGTTTTTTTTTTGTTTGAGACGGA | |
| | GTCTCACTCTGTCCCCCTTGCTGGAGTGCAGTGGCCGGATCT | |
| | CAGCTCACTGCAAGCTCCGCCTCCCGGGTTCAGGCAATTCTC | |
| | CTGCCTCAGCCTCCCGAGTAGCTGGGAATACAGGCGCCCGC | |
| | CACCTCGCCCAGCTAGTTTTTTTGTATTTTTTAGTAGAGACG | |
| | GGGTTTCACTGTGTTAGCCAGGATGTTCTCGATCTCCTGACC | |
| | TCGTGATCCGCCCGTCTCGGCCTCCCAAAGTGCTGGGATTAC | |
| | AGGCTTGAGCCACCGCGCCCAGCCTGTATTTTTTTTTTGAGA | |
| | TGGAGTCTTGCTCTGTCGCCCAGGCTAGAGTGCAGTGGTCG | |
| | GATCTCAGCTCACTGCAAGCTCCGCCTCCTGGGTTTACGCCA | |
| | TTCTCCTGCCTCAGCCTCCCGAGTAGCTGGGACTACAGGCGC | |
| | CCGCCACCTCGCCCGGCTAGTTTTTTGTATTTTTTAGTAGAG | |
| | ACGGTGTTTCACCGTGTTAGCCAGGATGGTCTCGATCTCCTG | |
| | ACCTCATGATCTGCCCATCTCGGCCTCCTGAAGTGCTGGGAT | |
| | TACAGGCTTGAGCCACTGTGCCCGGCCAATTTTTTGTATTTT | |
| | TAGTAGAGACGGGGTTTCATTATGTTGGCCAGGTTGGTCTCG | |
| | AACTCCTGACCTCGTGATCCACCCGTCTCGGCCTCCCAAAGT | |
| | GCTGGGATTACAGGCATGAGCCACAGGGCAGGGTCAGGCCT | |
| | AATTTTTGTATTTTTAGTAGAGATGGGGTTTCTCCATGTTGG | |
| | ACCAGGCTGGTCTTGAACTCCTAACTTCAGGTGATCCGCCTG | |
| | CCTTGGCCTCCCAAAGTACTGGGATTACAGGCGTGAGCCAC | |
| | CACACCTGGCCTAGACTTCAAGTCTTTCTTCCCTCGCTTCCA | |
| | ATACACTACTTTTCTGGGTCTTCACCTACCATTGCTTGTGCCT | |
| | GCCCACCAGCTTGGGTGGAGTCTTCCTTCCTCTCCAACTCCT | |
| | CACTCTTGGAGCCCTGGGCCCTCTTCTTATCCCTGTCTGCAC | |
| | GCTTTCCCATTTGACCTTGACTCCCAATGGCTTTTTGGGTCA | |
| | CTATGCCTTGGTGACTCTATTCCAGGCTCCATATTCAGCCGT | |
| | CCCCTGTGCCATTTGATGTCCTATGGACACCTCAGGCTCAAC | |
| | AGATACAAAATCAAACTCAATGTCTTCCCCAAGTATAGTCTT | |
| | CTTGGTGGCCCTGTGCAAGCAGAGGGCACCACCACCTGCTC | |
| | CCTTGCCCAGGCTAAGAACCTGGGCATCCTTCCTTTTCCTCA | |
| | CCCTGTCCAGCAAACTGGTCACAGTGTTCTGCCAGTTCTCTC | |
| | TCCTTCATACAATCCTATCATGCTATCCTAACCGCAATTTAC | |
| | AAACTCAACCCCAACTTTCACTCAAAACTTGATCCAAGCAA | |
| | TGTGCTGGATCCCAACTGTAACCTTGCAAACTCAACTCTGCC | |
| | CTTCACTTTGACTGTGACTGTCCTTAATTGCAGCAGGAAACT | |
| | GATCATTATGCTCTCCTCAATCCACACATTGCCTCTAAATAC | |
| | AGCCATGGTTTGCCTGTGATTTACTCAAAAGACACTGCCCCA | |
| | TGTCATGTGCCAGGCTCTGTGACAATCCCTGACCTCCTGGGA | |
| | CATGACTCCTTAGAGAGAGGAGAGCCTTTCTCACAGTTTGG | |
| | GACTTTGAGTCTGTCTTTTTTTTTTTCTCTTGAGACAGAGTTT | |
| | CGCTCTTGTTGCCCAGGCTGGAGTGCAGTGATCTCTGCTCAC | |
| | TGAAATCTCCACTCCCGGGTTCAAGCGATACTCCTGCCTCAG | |
| | CCTCCCAAGTAGCTGGGATTACAGGCGCCCGCCATCATACC | |
| | TAGCTAATTTTTTTGTATTTTTAGTAGAGACAGGGTTTCACC | |
| | ATGTTGGCCAGGCTGGTCTCGAACTCCTGACCTCAGGTGATC | |
| | CACCCGCCTTGGCCTCCCAAAGTGCTGGGATTACAGGCGTG | |
| | GACCACTGCACCTCGCCAAGTCTGTGTCTTGCCTCTGTGCCC | |
| | CAGACTTGCGGTTCTTTGAGATCTCGGGATTGGAACGTAAG | |
| | ATGCCAGCCTGGGGTCCTCATCTCATAGCCCCTTCCCCCTAG | |
| | CACTATGGCACTGGCTGCTGAGGATTGGTGGCTGGAAGGTC | |
| | CCAGCTGTGACTTTGACTTATCTGATCTTCTGCCTGTGTTCCC | |
| | TTGTGGGCATTCTTCAACTTCAAAGAGGTGAGTCATGTCCCC | |
| | AGTGGGTCTGTCCAAACCCTACACCATCTTCCCCAGGATAA | |
| | GCCGGCTTTGGCCAGTCTGACAACCATCTTTCCTTCCTCCCA | |
| | TCCCTCCCTGCAAGACCCCAGAATCCTGTTCCCCCCAGTCTT | |
| | CCTCTAGCCTCCCTCAAACTTCCGAAGCCTCTTGCATTTTTTT | |
| | TTTTTTTTTTTTGAGACAGGGTCTCATTCTGTCACCCCAGCTG | |
| | GAGTGCAGTGGCACAATCTGAGCTCACTGTAACCTCTGCCT | |
| | CCCAGGCTCAAGTGATTCTTGGGCCTCAGCCTCCTGAGTTGC | |
| | TGGGACTACAAGTGTGTGCCACCGCGCCCAGCTGATTTTTTA | |
| | TATTTTTAGTAGAGACAGGGTTTCGCCACATTGCCCACGCTG | |
| | GTCTTGAACTCCTGAGCTTAGGCAATTCGCTGGCCTCATCTT | |
| | CCTAAAGTGCTGGGAATATAGGCGTGAGCTACAGCGCCTGG | |
| | CTCCCTCAAACTTTTTTTTTTTTTTTTTTGAGATGGAGTTTTT | |
| | GCTCTGTTGCCCATGTTGGAGTGCAATGGCATGATCTCTGCT | |
| | CACTGCAACCTCCACCTCCTGGGTTCAAGCGATTCTCTTGCC | |
| | TCAGCCTCCCTAGTAGCTAGGATCACAGGCGCCCACCACCA | |
| | CGTCCGGCTAATTTTTATATTTTTAGTAAAGACGAAGTTTCA | |
| | CCATGTTGGCCAGGCTGGTCTCGAACTCTTGACTTCAAATGA | |
| | TCCGCCCACCTCGGCCTCCCAAAGTGCTGGGGTTACAGGCA | |
| | CTAGCCACCGCGCCCGTCCCCCTTGCAATTTGAACTCCTGTC | |
| | TTCTCTGTTGAACCAAGTGACCTCCGCAGTACCTGGCCCCAC | |
| | AAATCCTCACCCTGCCAAGCAGCGCCTCCTCTGATCACTCCC | |
| | TTTAACTCCCACCAGCCCTGGTCCTGAGAAGGAAAAGAAAG | |
| | CGAATGACTGACCCCACCAGGAGGTAATGCAACCAGTGCAC | |
| | CCCTCGGTAACACCCTCCACCTTCACTTTATGCCTTGCACTT | |
| | ACTGTTTCCTCTGCCCAGGGGTTCTTTGCTCCGTCTCTACTGT | |
| | TTCAAATACTGCCCAACCTCAAAGCCCAGCTCCAAAGCTAC | |
| | CTCCTCTGTGAAGACCTCCTTGGAAATGATCATCTCAGACTC | |
| | CTCTATTGGCTGTCCCAGCACAAGTGATCACGTTTAATCTCT | |
| | GAAGGCCTGGACAGAATCTTGAGTGGGTCCGCCATTCCATT | |
| | CCAAGTCGGCCCTCACCGTGCACTTGCCCTTCTCCCGCCAGG | |
| | TTCTTCAAAGTGACGCCTCCCCCAGGAAGCGGGCCCCAGAA | |
| | CCAGTATGGGAACGTGCTATCTCTCCCCACACCCACCTCAG | |
| | GCCTCGGTAAGAGGCACCGCCCCTCCAGCCTATAGCTCCGC | |
| | CCCAGATCCAGGGCTCCACCCACACTGTCCTCATCCCTCCAA | |
| | TCCGCTGTGCGCCATGCCTTCTGGAGCTCGGAACTCCGCCAC | |
| | CGGGGAGGGTAGTCCCGCCCATCTATGAGCCCCACCCCTAG | |
| | AACAATGCCCCGCCTCCAGGGCTCAGAGCCACGCCCCCAGG | |
| | ACCCAGAGCCTGAAGTCCAAATCAGAAGCAGAACCTCGCCC | |
| | CAGAACTGAAGGCCTTGGCCCTAGATTTAGATCCCGCCCTA | |
| | TGGTTCAAGGCCAGGTGCCTAGACTCAGAGTCCATTCGCAG | |
| | AGCCCAAAGCATTCTCTCCCCGTGCCCCGCCGCGCGGACCC | |
| | TTAGCCTTGACCGCCCCCATCTCTTCTGACCCCGTCTTACAA | |
| | CTCCCGCCCCTCTCGCCAGGACGCGCCCAGCGTTGGGCCGC | |
| | AGGCCTGGGGGGCACCGCCCCGTCTTATGGAAATCCAAGCA | |
| | GCGACGTCCAGGTGGATGGAGCCGTGGGGTCCCGGAGCCCT | |
| | CCGGGAGCGGGTGAATGATTGGGGGAGGGAAGGGTCGTTC | |
| | CCCACATAGAGGGGGTTGGAGAGGTCTGTGGCCCAAATAGT | |
| | GGACTGGGGCCTGGAGGAGAGGGGGCGTAACTCGGTTCCCC | |
| | ATCCCCATCCCGAAACCCCTAGGCCCAGAAGAAGAGGAAG | |
| | GGGAGGGCTATGAGGAGCCCGACAGTGAGGAGGGCTCCGA | |
| | GTTCTATGAGAACGACTCCAACTTTGGGCAGGACCAGCTCT | |
| | CCCAGGGTAAGGCTGCCCTCCTCCGTGGTCCCCCGCCTCTCT | |
| | GCGGTGGCCTGTGGACTCCCATGGACACCCCTCCTTCTCCAC | |
| | CAGATGGCAGCGGCTACGAGAACCCTGAGGACGAGCCCCTG | |
| | GGTCCTGAGGATGAAGACTCTTTCTCTAACGGTAACTTGGG | |
| | GCCTTTGTGGGACCTCAGAGACTTAGGTGTAATTGCAGCGC | |
| | TGTGACACCCCTAGAAGGGGTCCCTGGAGTTCTCTCTCTTCT | |
| | GCCACAGCTGAGTCTTACGAGAACGAGGATGAAGAGCTGAC | |
| | CCAGCCGGTCGCCAGGACAATGGGTGTGTGTGAGGATGGCA | |
| | ACAGTCTAGGGGGGAGGTGGAGGACGCCAGGAGGCCAGGA | |
| | GGAATAGTAACCTCCCTCTTCCCTTTCCAGACTTCCTGAGCC | |
| | CCCATGGGTCAGCCTGGGACCCCAGCCGGGAAGCAACTTCC | |
| | CTGGGTGAGAGATACTTTCGACCAGACTGCCTTACCCAGCTT | |
| | GGGTGACCTGGCCTGAGCTCTGACACCAGACCCAACTTTGA | |
| | CCTGACCTTGACCCCAAACCCGAACCCAATACTGACCTTGG | |
| | ATTTGACTCCTCTCACCTCAGTACTAAGCCCCATCCCCCATC | |
| | CTGACCCCCAATATTTACCCCCTCCCTAACTATGAATATCAA | |
| | CACCGATCCTGATCCAGTATCAAGCTGCACTTGACCTCCACC | |
| | TCACCTCAGCCCCTCAGTGCAGACCTCAACTTGGACCCCAG | |
| | CTCACTCTGCAGCTTCTTCATGACTCTGACTCCTTCCAGTTTT | |
| | TTCTTTTTTCTCTTTTTTTTGAATTGGAGTCTCGCTCTGTTGC | |
| | CCAGGCTGTAGTGCAGTTGCCACCTCTGCCTCCCAGGTTCAA | |
| | GCGATTCTCATGCCTCAGCCTCCTGAGTAGCTGGGATTACAG | |
| | ATGTTTGCCACCACACCTGGCTAATTTTTGTATCTTCAGTAG | |
| | AGACAGGGTTTCACCATGTTGGCCAGACTGGTCTCAGACTC | |
| | CTTACCTCTAATGATCTGCCCGCCTCGGCCTCCCGAAGTGCC | |
| | GGGATTACAGGTGTGAGCCACCATGCCCAGCCCAGTTCTGT | |
| | TCGTGACCCCTTCTCTAGCCATAATCTAACCCATATCTAGCC | |
| | GTGACTCTACAGCTAACCGGGGTCCCAAACCCGATGCTGAC | |
| | CGAATCACCCCTTCCCAGCACAGCATAGGTGATGCCCCTCA | |
| | CCTTCCTCTGCCCCTCATTCTTCCTCCTTACCATAGGCTGTAC | |
| | TTCCCGTGCCCTAGCCTCCAATTCTCCATCCCCCGCCCAAGC | |
| | AGGGTCCCAGTCCTATGAGGATATGAGAGGGCTCCTGTATG | |
| | CAGCCCCCCAGCTCCGCACCATTCGGGGCCAGCCTGGACCC | |
| | AATCATGAGGAAGGTGGGTGCTTCTGCCGCTGTCCCCTGCT | |
| | GTCCCCTGGGCTGACTTTGCCTCCCAGCCTACTTCCAGTGCC | |
| | ACCCGTGTTCTCCTCCCTGGTCCTATCCAGATGCAGACTCTT | |
| | ACGAGAACATGGATAATCCCGATGGGCCAGACCCAGCCTGG | |
| | GGAGGAGGCGGCCGCATGGGCACCTGGAGTGCCAGGTGAT | |
| | CCTCAGGTGGCCAGGTGAGCTGGGACTGCCCCTAGGGAAAG | |
| | GGGGGAGGGAGGGAGATAGGCACGAATGACAGTGGCTGCT | |
| | GGCTCTCAGGGAGGGAGAGGGAACAGGGCTCCTAGGGCCT | |
| | GGTGGGCAAGGGGAGGGCTGCTGGACCCCTCCCCATCACTG | |
| | TTTCTTCTTCATAGCCTGGATCTCTTCAAGTCCCCAAGATCC | |
| | ACACCTGACTCTGAAATCTGAAGACCTCGAGCAGATGATGC | |
| | CAACCTCTGGAGCAATGTTGCTTAGGATGTGTGCATGTGTGT | |
| | AAGTGTGTGTGTGTGGGTATACATGCCAGTGACACTTTCAGT | |
| | CCCTTTTGTATTCCTTAAATAAACTCAATGAGCTCTTCCAAT | |
| | CCTA | |
| 5 | QDGNEEMGGITQTPYKVSISGTTVILTCPQYPGSEILWQHNDKN | H. sapiens |
| | IGGDEDDKNIGSDEDHLSLKEFSELEQSGYYVCYPRGSKPEDA | CD3 epsilon |
| | NFYLYLRARVCENCMEMDVMSVATIVIVDICITGGLLLLVYY | amino acid (mature) |
| | WSKNRKAKAKPVTRGAGAGGRQRGQNKERPPPVPNPDYEPIR | sequence |
| | KGQRDLYSGLNQRRI | GenBank: |
| | | NP_000724.1 |
| 6 | TTCACCCCCAGGAGCCCATCCGGAAAGGCCAGCGGGACCTG | H. sapiens |
| | TATTCTGGCCTGAATCAGAGACGCATCTGACCTCTTGGAGA | CD3 |
| | ACACTGCCTCCCGCTGGCCCAGGTCTCCTCTCCAGTCCCCCT | nucleotide |
| | GCGACTCCCTGTTTCCTGGGCTAGTCTTGGACCCCACGAGAG | sequence |
| | AGAATCGTTCCTCAGCCTCATGGTGAACTCGCGCCCTCCAGC | GenBank: |
| | CTGATCCCCCGCTCCCTCCTCCCTGCCTTCTCTGCTGGTACCC | AH002678.2 |
| | AGTCCTAAAATATTGCTGCTTCCTCTTCCTTTGAAGCATCAT | |
| | CAGTAGTCACACCCTCACAGCTGGCCTGCCCTCTTGCCAGG | |
| | ATATTTATTTGTGCTATTCACTCCCTTCCCTTTGGATGTAACT | |
| | TCTCCGTTCAGTTCCCTCCTTTTCTTGCATGTAAGTTGGTCCC | |
| | CCATCCCAAAGTATTCCATCTACTTTTCTATCGCCGTCCCCTT | |
| | TTGCAGCCCTCTCTGGGGATGGACTGGGTAAATGTTGACAG | |
| | AGGCCCTGCCCCGTTCACAGATCCTGGCCCTGAGCCAGCCC | |
| | TGTGCTCCTCCCTCCCCCAACACTCCCTACCAACCCCCTAAT | |
| | CCCCTACTCCCTCCAACCCCCCCTCCCACTGTAGGCCACTGG | |
| | ATGGTCATTTGGCATCTCCGTATATGTGCTCTGGCTCCTCAG | |
| | CTGAGAGAGAAAAAAATAAACTGTATTTGGCTGCAAAGAGT | |
| | T | |
| 7 | MQSGTRWRVLGLCLLSIGVWGQDGNEEMGSITQTPYQVSISGT | Macaca |
| | TVILTCSQHLGSEAQWQHNGKNKEDSGDRLFLPEFSEMEQSGY | fascicularis |
| | YVCYPRGSNPEDASHHLYLKARVCENCMEMDVMAVATIVIVD | CD3 |
| | ICITLGLLLLVYYWSKNRKAKAKPVTRGAGAGGRQRGQNKER | amino acid |
| | PPPVPNPDYEPIRKGQQDLYSGLNQRRI | sequence |
| | | GenBank: |
| | | BAB71849.1 |
| 8 | ATGCAGTCGGGCACTCGCTGGAGAGTTCTGGGCCTCTGCCT | Macaca |
| | CTTATCAATTGGCGTTTGGGGGCAAGATGGTAATGAAGAAA | fascicularis |
| | TGGGTAGTATTACACAGACACCATATCAAGTCTCCATCTCTG | CD3 |
| | GAACCACAGTAATACTGACATGCTCTCAGCATCTTGGATCT | nucleotide |
| | GAAGCACAATGGCAACACAATGGTAAAAACAAAGAAGATT | sequence |
| | CTGGGGATCGGCTGTTTCTGCCGGAATTTTCAGAAATGGAG | GenBank: |
| | CAAAGTGGTTATTATGTCTGCTACCCCAGAGGAAGCAATCC | AB073993.1 |
| | AGAGGACGCGAGCCATCATCTCTACCTGAAGGCAAGAGTGT | |
| | GTGAGAACTGCATGGAGATGGATGTGATGGCGGTGGCCACA | |
| | ATTGTCATAGTGGACATCTGCATCACTCTGGGCTTGCTGCTG | |
| | CTGGTTTACTACTGGAGCAAGAATAGAAAGGCCAAGGCCAA | |
| | GCCTGTGACACGAGGAGCAGGTGCTGGCGGCAGGCAAAGG | |
| | GGACAAAACAAGGAGAGGCCACCACCTGTTCCCAACCCAG | |
| | ACTATGAGCCCATCCGGAAAGGCCAGCAGGATCTGTATTCT | |
| | GGCCTGAATCAGAGACGCATCTGA | |
| 9 | DYYWS | 1, 11, 12, 13-CD19 |
| | | HCDR1 amino acid |
| | | sequence |
| 10 | EITHSGSTNYNPSLKS | 1, 11, 12, 13-CD19 |
| | | HCDR2 amino acid |
| | | sequence |
| 11 | GGLDIFTGPYYFDS | 1, 11, 12, 13-CD19 |
| | | HCDR3 amino acid |
| | | sequence |
| 12 | QVQLQQWGAGLLKPSETLSLTCAVYGGSFSDYYWSWVRQPP | 13-CD19 |
| | GKCLEWIGEITHSGSTNYNPSLKSRVTISVDTSKNQFSLKLSSVT | VH amino acid |
| | AADTAVYYCARGGLDIFTGPYYFDSWGQGTLVTVSS | sequence |
| 13 | CAAGTGCAGCTTCAGCAGTGGGGCGCCGGTCTGCTGAAGCC | 13-CD19 |
| | TTCCGAAACCCTCTCGCTGACTTGCGCCGTCTACGGCGGGA | VH nucleotide |
| | GCTTCAGCGATTACTACTGGAGCTGGGTTCGCCAGCCGCCG | sequence |
| | GGAAAGTGTCTGGAGTGGATCGGAGAGATTACGCATTCCGG | |
| | ATCCACCAACTACAATCCTAGCCTGAAGTCAAGAGTGACCA | |
| | TTTCTGTGGACACCTCCAAGAACCAGTTTTCCCTGAAACTGT | |
| | CCTCCGTGACTGCCGCGGATACCGCGGTGTACTACTGCGCA | |
| | CGCGGGGGACTGGACATCTTCACCGGACCGTATTACTTCGA | |
| | CTCCTGGGGACAGGGAACTCTGGTCACCGTCAGCTCC | |
| 14 | RASQEIRKDLA | 1, 11, 12, 13-CD19 |
| | | LCDR1 amino acid |
| | | sequence |
| 15 | GASSLRS | 1, 11, 12, 13-CD19 |
| | | LCDR2 amino acid |
| | | sequence |
| 16 | QQDYNYPPT | 1, 11, 12, 13-CD19 |
| | | LCDR3 amino acid |
| | | sequence |
| 17 | DIQMTQSPSSLSASVGDRVTITCRASQEIRKDLAWYQQKPGKA | 13-CD19 |
| | PKLLIYGASSLRSGVPSRFSGSGSGTDFTLTISSLQPEDFATYYC | VL amino acid |
| | QQDYNYPPTFGCGTKVEIK | sequence |
| 18 | GACATCCAGATGACCCAATCCCCTTCCTCGCTGAGCGCCTCC | 13-CD19 |
| | GTCGGGGACAGAGTCACCATCACATGCCGCGCAAGCCAAGA | VL nucleotide |
| | GATCCGGAAGGACCTGGCCTGGTACCAGCAGAAACCCGGA | sequence |
| | AAGGCTCCGAAGCTGCTGATCTACGGAGCTTCCTCTCTCCGG | |
| | TCGGGCGTGCCTAGCCGGTTCAGCGGTAGCGGATCCGGAAC | |
| | TGATTTCACCCTGACCATTTCCTCGCTGCAACCCGAGGACTT | |
| | CGCCACCTACTACTGCCAACAAGACTACAACTATCCACCTA | |
| | CGTTTGGGTGCGGCACCAAGGTCGAAATCAAG | |
| 19 | RYGIH | 1, 3, 4, 5, 6, 7, 8, |
| | | 9, 10, 11, 12, 13, |
| | | 14, 15, 16- |
| | | CD3-VH-CDR1 |
| | | HCDR1 amino acid |
| | | sequence |
| 20 | AIWYDASKTYYADSVKG | 5, 6, 7, 8, 12, 16- |
| | | CD3-VH-CDR2 |
| 21 | SNYDILTRYYQYGLDV | 3, 5, 6, 9, 10, 11, |
| | | 12, 13, 14, 15- |
| | | CD3-VH-CDR3 |
| 22 | QVQLVESGGGVVQPGGSLRLSCAASGFTFSRYGIHWVRQAPG | 5-CD3 |
| | KCLEWVAAIWYDASKTYYADSVKGRFTISRDNSKNTLYLQMN | VH amino acid |
| | SLRAEDTAVYYCARSNYDILTRYYQYGLDVWGQGTTVTVSS | sequence |
| 23 | CAAGTGCAGCTCGTCGAATCCGGCGGCGGAGTGGTGCAGCC | 5-CD3 |
| | TGGCGGATCGCTGCGGCTGTCATGCGCGGCTTCCGGCTTCAC | VH nucleotide |
| | CTTCTCGCGCTACGGGATCCACTGGGTCAGACAGGCCCCCG | sequence |
| | GAAAGTGCCTCGAATGGGTGGCCGCCATTTGGTACGACGCA | |
| | TCCAAGACCTATTACGCGGATAGCGTGAAGGGCCGCTTCAC | |
| | TATCTCCCGGGACAACAGCAAGAACACTCTGTACCTCCAAA | |
| | TGAATAGCCTGAGGGCTGAGGACACCGCCGTGTACTACTGC | |
| | GCCCGCTCCAACTACGACATTCTGACCCGGTACTATCAGTAC | |
| | GGGCTTGACGTCTGGGGACAGGGGACCACCGTGACTGTGTC | |
| | ATCC | |
| 24 | RASQGISSALA | 1, 2, 3, 4, 5, 6, 7, |
| | | 8, 9, 10, 11, 12, 13, |
| | | 14, 15, 16- |
| | | CD3-VL-CDR1 |
| 25 | DASSLES | 2, 3, 4, 5, 6, 7, 8, |
| | | 9, 10, 11, 12, 13, |
| | | 14, 15, 16- |
| | | CD3-VL-CDR2 |
| | | 5-CD19-VL-CDR2 |
| 26 | QQFKSYPRT | 1, 2, 3, 5, 6-CD3- |
| | | VL-CDR3 |
| 27 | AIQLTQSPSSLSASVGDRVTITCRASQGISSALAWYQQKPGKAP | 5-CD3-VL |
| | KLLIYDASSLESGVPSRFSGSGSGTDFTLTISSLQPEDFATYYCQ | VL amino acid |
| | QFKSYPRTFGCGTKVEIK | sequence |
| 28 | GCTATCCAACTCACCCAATCACCAAGCTCCCTGTCCGCGTCC | 5-CD3 |
| | GTGGGCGATAGAGTGACCATCACTTGCCGGGCCAGCCAGGG | VL nucleotide |
| | CATTTCTTCGGCCCTGGCCTGGTACCAGCAGAAGCCTGGAA | sequence |
| | AAGCCCCGAAGCTGCTGATCTACGACGCAAGCTCATTGGAG | |
| | AGCGGAGTGCCGTCCCGCTTCTCCGGATCCGGTTCGGGGAC | |
| | CGACTTCACTCTGACCATTTCCTCCCTGCAACCGGAAGATTT | |
| | CGCGACATACTACTGTCAACAATTCAAGTCGTACCCCCGGA | |
| | CGTTCGGATGCGGGACCAAAGTCGAGATCAAG | |
| 29 | PEEPLVVKVEEGDNAVLQCLKGTSDGPTQQLTWSRESPLKPFL | CD19-Alb2-12xHis |
| | KLSLGLPGLGIHMRPLAIWLFIFNVSQQMGGFYLCQPGPPSEKA | construct amino acid |
| | WQPGWTVNVEGSGELFRWNVSDLGGLGCGLKNRSSEGPSSPS | sequence |
| | GKLMSPKLYVWAKDRPEIWEGEPPCLPPRDSLNQSLSQDLTM | |
| | APGSTLWLSCGVPPDSVSRGPLSWTHVHPKGPKSLLSLELKDD | |
| | RPARDMWVMETGLLLPRATAQDAGKYYCHRGNLTMSFHLEI | |
| | TARPVLWHWLLRTGGWKGGGGSGGGGSGSAAADEGKASSA | |
| | KQRLKCASLQKFGERAFKAWAVARLSQRFPKAEFAEVSKLVT | |
| | DLTKVHTECCHGDLLECADDRADLAKYICENQDSISSKLKECC | |
| | EKPLLEKSHCIAEVENDEMPADLPSLAADFVESKDVCKNYAEA | |
| | KDVFLGMFLYEYARRHPDYSVVLLLRLAKTYETTLEKCCAAA | |
| | DPHECYAKVFDEFKPGTSHHHHHHHHHHHH | |
| 30 | EVKLQESGPGLVAPSQSLSVTCTVSGVSLPDYGVSWIRQPPRK | FMC63 VH |
| | GLEWLGVIWGSETTYYNSALKSRLTIIKDNSKSQVFLKMNSLQ | |
| | TDDTAIYYCAKHYYYGGSYAMDYWGQGTSVTVSS | |
| 31 | DIQMTQTTSSLSASLGDRVTISCRASQDISKYLNWYQQKPDGT | FMC63 VL |
| | VKLLIYHTSRLHSGVPSRFSGSGSGTDYSLTISNLEQEDIATYFC | |
| | QQGNTLPYTFGGGTKLEIT | |
| 32 | QVQLQQWGAGLLKPSETLSLTCAVYGGSFSDYYWSWVRQPP | 1-CD19-VH |
| | GKGLEWIGEITHSGSTNYNPSLKSRVTISVDTSKNQFSLKLSSVT | |
| | AADTAVYYCARGGLDIFTGPYYFDSWGQGALVTVSS | |
| 33 | EIVLTQSPSSLSASVGDRVTITCRASQEIRKDLAWYQQKPGKAP | 1-CD19-VL |
| | KVLIYGASSLRSGVPSSFSGSGSGTDFTLTISSLQPEDFATYYCQ | |
| | QDYNYPPTFGQGTKVEIK | |
| 34 | QVQLVQSGAEVKKPGESLKISCKGSGYSFTSYWIGWVRQMPG | 2-CD19-VH |
| | KGLELMGIIYPGDSDTRYSPSFQGQVTISADKSISTAYLQWSSL | |
| | KASDTAMYYCARGGTTIFGVVFEYWGQGTLVTVSS | |
| 35 | EIVLTQSPSSLSTSVGDRVTITCRASQSINNYLNWYQQKPGKAP | 2-CD19-VL |
| | KLLIYGASSLQSGVPSRFSGSGSGTDFTLTISSLQPEDFATYYCQ | |
| | QTYSTPWTFGQGTKVEIK | |
| 36 | QVQLVESGGGVVQPGRSLRLSCAASGFTFSRYGIHWVRQAPG | 1-CD3-VH |
| | KGLEWVAAIWYDGSKKYYADSVKGRFTISRDNSKNTLDLQM | |
| | NSLRAEDTAVYYCARSNYDILTRYYQYGINVWGQGTTVTVSS | |
| 37 | AIQLTQSPSSLSASVGDRVTITCRASQGISSALAWYQQKPGKAP | 1-CD3-VL |
| | KLLIYDASSLENGVPSRFSGSGSGTDFTLTISSLQPEDFATYYCQ | |
| | QFKSYPRTFGQGTKVEIK | |
| 38 | QVQLVESGGGVVQPGRSLRLSCAASGFTFSNYGIHWVRQAPG | 2-CD3-VH |
| | KGLEWVAAIWYDASKKYYADSVKGRFTISRDNSKNTLYLQM | |
| | NSLRAEDTAVYYCARSNYDILTRYYQYGINVWGQGTTVTVSS | |
| 39 | AIQLTQSPSSLSASVGDRVTITCRASQGISSALAWYQQKPGKAP | 2, 3, 6, 9, 10, 11, |
| | KLLIYDASSLESGVPSRFSGSGSGTDFTLTISSLQPEDFATYYCQ | 12, 13, 14, 15-CD3- |
| | QFKSYPRTFGQGTKVEIK | VL |
| 40 | QVQLVESGGGVVQPGRSLRLSCAASGFTFSRYGIHWVRQAPG | 3-CD3-VH |
| | KGLEWVAAIWYDASKKYYADSVKGRFTISRDNSKNTLYLQM | |
| | NSLRAEDTAVYYCARSNYDILTRYYQYGLDVWGQGTTVTVSS | |
| 41 | QVQLVESGGGVVQPGRSLRLSCAASGFTFSRYGIHWVRQAPG | 1-CD3-M1-VH |
| | KGLEWVAAIWYDGSKKYYADSVKGRFTISRDNSKNTLYLQM | |
| | NSLRAEDTAVYYCARSNYDILTRYYQYGINVWGQGTTVTVSS | |
| 42 | QVQLVESGGGVVQPGRSLRLSCAASGFTFSRYGIHWVRQAPG | 4-CD3-VH |
| | KGLEWVAAIWYDASKKYYADSVKGRFTISRDNSKNTLYLQM | |
| | NSLRAEDTAVYYCARSNYDILTRYYSHGINVWGQGTTVTVSS | |
| 43 | AIQLTQSPSSLSASVGDRVTITCRASQGISSALAWYQQKPGKAP | 4, 7, 16-CD3-VL |
| | KLLIYDASSLESGVPSRFSGSGSGTDFTLTISSLQPEDFATYYCQ | |
| | QFNSYPRTFGQGTKVEIK | |
| 44 | EVQLVQSGAEVKKPGESLKISCKGSGYSFTSYWIGWVRQMPG | 3-CD19-VH |
| | KGLEWMGIIYPGDSDTRYSPSFQGQVTISADKSLSTAYLQWSG | |
| | LKASDTAMYYCARQSIMGVIYVAFDVWGQGTMVTVSS | |
| 45 | EIVLTQSPSSLSASVGDRVTITCRASQSISSYLNWYQQKPGKAP | 3-CD19-VL |
| | KLLIYAASSLQSGVPSRFSGSGSGTDFTLTITSLRPEDFATYYCQ | |
| | QSYSNPWTFGQGTKVEIK | |
| 46 | EVQLVQSGAEVKKPGESLKISCKGSGYSFTSYWIGWVRQMPG | 4-CD19-VH |
| | KGLEWMGIIYPGDSDTRYSPSFQGQVTISADKSISTAYLQWSSL | |
| | KASDTAMYYCARGGATIFGVVFDYWGQGTLVTVSS | |
| 47 | EIVLTQSPSSLSASVGDRVTITCRASQSINSYLSWFQQKPGKAPK | 4-CD19-VL |
| | LLIYAASSLQSGVPSRFSGSGSGTDFTLTISSLQPEDFATYYCQQ | |
| | SYSTPPTFGQGTKVEIK | |
| 48 | QVQLQESGPGLVKPSETLSLTCTVSGGSISSYYWSWIRQPPGKG | 5-CD19-VH |
| | LEWIGYIYYSGSTKYNPSLKSRVTISVDTSKNQFSLQLSSVTAA | |
| | DTAVYYCATGLAAAGDYYYGMDVWGQGTTVTVSS | |
| 49 | EIVLTQSPSSLSASVGDRVTITCRASQGISSALAWYQQKPGKAP | 5-CD19-VL |
| | KLLIYDASSLESGVPSRFSGGGSGTDFTLTISSLQPEDFASYYCQ | |
| | QFNSYPLTFGGGTKVEIK | |
| 50 | QVQLVQSGAEVKKPGESLKISCKGSGYSFTSYWIGWVRQMPG | 6-CD19-VH |
| | KGLEWMGIIYPGDSDTRYSPSFQGQVTISADKSISTAYLQWSSL | |
| | KASDTAMYYCARQSIVATITWAFDIWGQGTMVTVSS | |
| 51 | EIVLTQSPSFLSASVGDRVTITCRASQGISSYLAWYQQKPGKAP | 6-CD19-VL |
| | KLLIYAASTLQSGVPSRFSGSGSGTEFTLTVSSLQPEDFATYYC | |
| | QQLNSYPYTFGQGTKLEIK | |
| 52 | QVQLQESGPGLVKPSETLSLTCTVSGGSISSGSYNWGWIRQPPG | 7-CD19-VH |
| | KGLEWIGSIYYSGSTYYNPSLKSRVTISGDTSKNQFSLKLSSLTA | |
| | ADTAVYYCARHTGGFDYWGQGTLVTVSS | |
| 53 | EIVLTQSPATLSVSPGERATLSCRASQSVSSNLAWYQQKPGQAP | 7-CD19-VL |
| | RLLIYGASIRATGIPARFSGSGSGTEFTLTISSLQSEDFAVYYCQ | |
| | QYNNWPLTFGGGTKVEIK | |
| 54 | QVQLVQSGAEVKKPGESLRISCKGSGYSFSSYWIAWVRQMPG | 8-CD19-VH |
| | KGLEWMGIIYPGDSDTRYSPSFQGQVTISADKSISTAYLQWSSL | |
| | KASDTAMYYCARHGGYNYGFTDYYYGMDVWGQGTTVTVSS | |
| 55 | DIVMTQTPLSSPVTLGQPASISCRSSQRLVHSDGDTYLSWLQQR | 8-CD19-VL |
| | PGQPPRLLIYKISNRFSGVPDRFSGSGAGTDFTLKISRVEAEDVG | |
| | VYYCMQATQFPYTFGQGTKLEIK | |
| 56 | EVQLVQSGAEVKKPGESLKISCKGSGYSFTSYWIGWVRQMPG | 9-CD19-VH |
| | KGLEWMGIIYPGDSDTRYSPSFQGQVAISADKSISTAYLQWSSL | |
| | KASDTAMYYCARRSIAGTTSWSFDLWGRGTLVTVSS | |
| 57 | EIVLTQSPSSLSASVGDRVTITCRTSQDIRNDLGWYQQKPGNAP | 9-CD19-VL |
| | KLLIYAASSLQSGVPSRFSGSGSGTDFTLTISSLQPEDFATYYCL | |
| | QDYNYPLTFGGGTKVEIK | |
| 58 | QVQLQESGPGLVKPSETLSLTCTVSGGSFSSYYWSWIRQPPGK | 10-CD19-VH |
| | GLEWIGYIYYSGRTNYNPSLKSRVTISIDTSKNQVSLKLSSVTA | |
| | ADTAVYYCASHHWGSALDYWGQGTLVTVSS | |
| 59 | EIVLTQSPLSLPVTLGQPASISCRSSQSLVYSDGNTYLNWFQQR | 10-CD19-VL |
| | PGQSPRRLIYKVSNRDSGVPDRFSGSGSGTDFTLKISRVEAEDV | |
| | GVYYCMQGTHWPWTFGQGTKVEIK | |
| 60 | QVQLQQWGAGLLKPSETLSLTCAVYGGSFSDYYWSWVRQPP | 11-CD19-VH |
| | GKGLEWIGEITHSGSTNYNPSLKSRVTISVDTSKNQFSLKLSSVT | |
| | AADTAVYYCARGGLDIFTGPYYFDSWGQGTLVTVSS | |
| 61 | DIQMTQSPSSLSASVGDRVTITCRASQEIRKDLAWYQQKPGKA | 11-CD19-VL |
| | PKLLIYGASSLRSGVPSRFSGSGSGTDFTLTISSLQPEDFATYYC | |
| | QQDYNYPPTFGQGTKVEIK | |
| 62 | QVQLQQWGAGLLKPSETLSLTCAVYGGSFSDYYWSWVRQPP | 12-CD19-VH |
| | GKCLEWIGEITHSGSTNYNPSLKSRVTISVDTSKNQFSLKLSSVT | |
| | AADTAVYYCARGGLDIFTGPYYFDSWGQGALVTVSS | |
| 63 | EIVLTQSPSSLSASVGDRVTITCRASQEIRKDLAWYQQKPGKAP | 12-CD19-VL |
| | KVLIYGASSLRSGVPSSFSGSGSGTDFTLTISSLQPEDFATYYCQ | |
| | QDYNYPPTFGCGTKVEIK | |
| 64 | QVQLVESGGGVVQPGRSLRLSCAASGFTFSRYGIHWVRQAPG | 1-CD3-M2-VH |
| | KGLEWVAAIWYDSSKKYYADSVKGRFTISRDNSKNTLDLQMN | |
| | SLRAEDTAVYYCARSNYDILTRYYQYGINVWGQGTTVTVSS | |
| 65 | QVQLVESGGGVVQPGRSLRLSCAASGFTFSRYGIHWVRQAPG | 1-CD3-M3-VH |
| | KGLEWVAAIWYDASKKYYADSVKGRFTISRDNSKNTLDLQM | |
| | NSLRAEDTAVYYCARSNYDILTRYYQYGINVWGQGTTVTVSS | |
| 66 | QVQLVESGGGVVQPGRSLRLSCAASGFTFSRYGIHWVRQAPG | 1-CD3-M5-VH |
| | KGLEWVAAIWYDGSHKYYADSVKGRFTISRDNSKNTLDLQM | |
| | NSLRAEDTAVYYCARSNYDILTRYYQYGINVWGQGTTVTVSS | |
| 67 | QVQLVESGGGVVQPGRSLRLSCAASGFTFSRYGIHWVRQAPG | 1-CD3-M6-VH |
| | KGLEWVAAIWYDGSNKYYADSVKGRFTISRDNSKNTLDLQM | |
| | NSLRAEDTAVYYCARSNYDILTRYYQYGINVWGQGTTVTVSS | |
| 68 | QVQLVESGGGVVQPGGSLRLSCAASGFTFSRYGIHWVRQAPG | 6-CD3-VH |
| | KGLEWVAAIWYDASKTYYADSVKGRFTISRDNSKNTLYLQMN | |
| | SLRAEDTAVYYCARSNYDILTRYYQYGLDVWGQGTTVTVSS | |
| 69 | QVQLVESGGGVVQPGRSLRLSCAASGFTFSRYGIHWVRQAPG | 1-CD3-M7-VH |
| | KGLEWVAAIWYDGSKKYYADSVKGRFTISRDNSKNTLDLQM | |
| | NSLRAEDTAVYYCARSNYDILTRHYQYGINVWGQGTTVTVSS | |
| 70 | QVQLVESGGGVVQPGGSLRLSCAASGFTFSRYGIHWVRQAPG | 7-CD3-VH |
| | KGLEWVAAIWYDASKTYYADSVKGRFTISRDNSKNTLYLQMN | |
| | SLRAEDTAVYYCARSNYDILTRYYSHGINVWGQGTTVTVSS | |
| 71 | QVQLVESGGGVVQPGRSLRLSCAASGFTFSRYGIHWVRQAPG | 1-CD3-M8-VH |
| | KGLEWVAAIWYDGSKKYYADSVKGRFTISRDNSKNTLDLQM | |
| | NSLRAEDTAVYYCARSNYDILTGHYQYGINVWGQGTTVTVSS | |
| 72 | QVQLVESGGGVVQPGGSLRLSCAASGFTFSRYGIHWVRQAPG | 8-CD3-VH |
| | KCLEWVAAIWYDASKTYYADSVKGRFTISRDNSKNTLYLQMN | |
| | SLRAEDTAVYYCARSNYDILTRYYSHGINVWGQGTTVTVSS | |
| 73 | AIQLTQSPSSLSASVGDRVTITCRASQGISSALAWYQQKPGKAP | 8-CD3-VL |
| | KLLIYDASSLESGVPSRFSGSGSGTDFTLTISSLQPEDFATYYCQ | |
| | QFNSYPRTFGCGTKVEIK | |
| 74 | QVQLVESGGGVVQPGGSLRLSCAASGFTFSRYGIHWVRQAPG | 9-CD3-VH |
| | KGLEWVAAIWYDASKKYYADSVKGRFTISRDNSKNTLYLQM | |
| | NSLRAEDTAVYYCARSNYDILTRYYQYGLDVWGQGTTVTVSS | |
| 75 | QVQLVESGGGVVQPGRSLRLSCAASGFTFSRYGIHWVRQAPG | 1-CD3-M9-VH |
| | KGLEWVAAIWYDGSKKYYADSVKGRFTISRDNSKNTLDLQM | |
| | NSLRAEDTAVYYCARSNYDILTGYYQYGINVWGQGTTVTVSS | |
| 76 | QVQLVESGGGVVQPGRSLRLSCAASGFTFSRYGIHWVRQAPG | 1-CD3-M10-VH |
| | KGLEWVAAIWYDGSKKYYADSVKGRFTISRDNSKNTLDLQM | |
| | NSLRAEDTAVYYCARSNYDILTRYYQHGINVWGQGTTVTVSS | |
| 77 | QVQLVESGGGVVQPGRSLRLSCAASGFTFSRYGIHWVRQAPG | 1-CD3-M11-VH |
| | KGLEWVAAIWYDGSKKYYADSVKGRFTISRDNSKNTLDLQM | |
| | NSLRAEDTAVYYCARSNYDILTRYYSHGINVWGQGTTVTVSS | |
| 78 | QVQLVESGGGVVQPGESLRLSCAASGFTFSRYGIHWVRQAPG | 11-CD3-VH |
| | KGLEWVAAIWYDASKDYYADSVKGRFTISRDNSKNTLYLQM | |
| | NSLRAEDTAVYYCARSNYDILTRYYQYGLDVWGQGTTVTVSS | |
| 79 | QVQLVESGGGVVQPGRSLRLSCAASGFTFSNYGIHWVRQAPG | 1-CD3-M16-VH |
| | KGLEWVAAIWYDGSKKYYADSVKGRFTISRDNSKNTLDLQM | |
| | NSLRAEDTAVYYCARSNYDILTRYYQYGINVWGQGTTVTVSS | |
| 80 | QVQLVESGGGVVQPGGSLRLSCAASGFTFSRYGIHWVRQAPG | 12-CD3-VH |
| | KGLEWVAAIWYDASKTYYADSVKGRFTISRDNSSNTLYLQMN | |
| | SLRAEDTAVYYCARSNYDILTRYYQYGLDVWGQGTTVTVSS | |
| 81 | QVQLVESGGGVVQPGRSLRLSCAASGFTFSNHGIHWVRQAPG | 1-CD3-M17-VH |
| | KGLEWVAAIWYDGSKKYYADSVKGRFTISRDNSKNTLDLQM | |
| | NSLRAEDTAVYYCARSNYDILTRYYQYGINVWGQGTTVTVSS | |
| 82 | QVQLVESGGGVVQPGESLRLSCAASGFTFSRYGIHWVRQAPG | 13-CD3-VH |
| | KGLEWVAAIWYDASKDYYADSVKGRFTISRDNSENTLYLQMN | |
| | SLRAEDTAVYYCARSNYDILTRYYQYGLDVWGQGTTVTVSS | |
| 83 | QVQLVESGGGVVQPGRSLRLSCAASGFTFSRHGIHWVRQAPG | 1-CD3-M18-VH |
| | KGLEWVAAIWYDGSKKYYADSVKGRFTISRDNSKNTLDLQM | |
| | NSLRAEDTAVYYCARSNYDILTRYYQYGINVWGQGTTVTVSS | |
| 84 | QVQLVESGGGVVQPGGSLRLSCAASGFTFSRYGIHWVRQAPG | 14-CD3-VH |
| | KGLEWVAAIWYDASKKYYADSVKGRFTISRDNSSNTLYLQMN | |
| | SLRAEDTAVYYCARSNYDILTRYYQYGLDVWGQGTTVTVSS | |
| 85 | QVQLVESGGGVVQPGRSLRLSCAASGFTFSRYGIHWVRQAPG | 1-CD3-M19-VH |
| | KGLEWVAAIWYDGSKKYYADSVKGRFTISRDNSKNTLDLQM | |
| | NSLRAEDTAVYYCARSNFDILTRYYQYGINVWGQGTTVTVSS | |
| 86 | QVQLVESGGGVVQPGESLRLSCAASGFTFSRYGIHWVRQAPG | 15-CD3-VH |
| | KGLEWVAAIWYDASKKYYADSVKGRFTISRDNSENTLYLQMN | |
| | SLRAEDTAVYYCARSNYDILTRYYQYGLDVWGQGTTVTVSS | |
| 87 | QVQLVESGGGVVQPGRSLRLSCAASGFTFSRYGIHWVRQAPG | 1-CD3-M20-VH |
| | KGLEWVAAIWYDGSKKYYADSVKGRFTISRDNSKNTLDLQM | |
| | NSLRAEDTAVYYCARSNFDVLTRYYQYGINVWGQGTTVTVSS | |
| 88 | EVQLVQSGAEVKKPGESLKISCKGSGYSFTSYWIGWVRQMPG | 14-CD19-VH |
| | KCLEWMGIIYPGDSDTRYSPSFQGQVTISADKSISTAYLQWSSL | |
| | KASDTAMYYCARGGTTIFGVVFEYWGQGTLVTVSS | |
| 89 | DIQMTQSPSSLSASVGDRVTITCRASQSINNYLNWYQQKPGKA | 14-CD19-VL |
| | PKLLIYGASSLQSGVPSRFSGSGSGTDFTLTISSLQPEDFATYYC | |
| | QQTYSTPWTFGCGTKVEIK | |
| 90 | QVQLVESGGGVVQPGRSLRLSCAASGFTFSRYGIHWVRQAPG | 1-CD3-M21-VH |
| | KGLEWVAAIWYDGSKKYYADSVKGRFTISRDNSKNTLDLQM | |
| | NSLRAEDTAVYYCARSNYDVLTRYYQYGINVWGQGTTVTVSS | |
| 91 | QVQLVESGGGVVQPGRSLRLSCAASGFTFSRYGIHWVRQAPG | 1-CD3-M22-VH |
| | KGLEWVAAIWYDGSKKYYADSVKGRFTISRDNSKNTLDLQM | |
| | NSLRAEDTAVYYCARSNYDILTRYYQYGLDVWGQGTTVTVSS | |
| 92 | QVQLVESGGGVVQPGRSLRLSCAASGFTFSRYGIHWVRQAPG | 1-CD3-C1-VH |
| | KGLEWVAAIWYDASKKYYADSVKGRFTISRDNSKNTLYLQM | |
| | NSLRAEDTAVYYCARSNYDILTRYYQYGINVWGQGTTVTVSS | |
| 93 | QVQLVESGGGVVQPGRSLRLSCAASGFTFSRYGIHWVRQAPG | 1-CD3-C5-VH |
| | KGLEWVAAIWYDASHKYYADSVKGRFTISRDNSKNTLYLQM | |
| | NSLRAEDTAVYYCARSNYDILTRYYQYGINVWGQGTTVTVSS | |
| 94 | QVQLVESGGGVVQPGRSLRLSCAASGFTFSRYGIHWVRQAPG | 1-CD3-C6-VH |
| | KGLEWVAAIWYDASNKYYADSVKGRFTISRDNSKNTLYLQM | |
| | NSLRAEDTAVYYCARSNYDILTRYYQYGINVWGQGTTVTVSS | |
| 95 | QVQLVESGGGVVQPGRSLRLSCAASGFTFSRYGIHWVRQAPG | 1-CD3-C8-VH |
| | KGLEWVAAIWYDASKKYYADSVKGRFTISRDNSKNTLYLQM | |
| | NSLRAEDTAVYYCARSNYDILTGHYQYGINVWGQGTTVTVSS | |
| 96 | QVQLVESGGGVVQPGRSLRLSCAASGFTFSRYGIHWVRQAPG | 1-CD3-C9-VH |
| | KGLEWVAAIWYDASKKYYADSVKGRFTISRDNSKNTLYLQM | |
| | NSLRAEDTAVYYCARSNYDILTGYYQYGINVWGQGTTVTVSS | |
| 97 | QVQLVESGGGVVQPGRSLRLSCAASGFTFSNHGIHWVRQAPG | 1-CD3-C17-VH |
| | KGLEWVAAIWYDASKKYYADSVKGRFTISRDNSKNTLYLQM | |
| | NSLRAEDTAVYYCARSNYDILTRYYQYGINVWGQGTTVTVSS | |
| 98 | QVQLVESGGGVVQPGRSLRLSCAASGFTFSRYGIHWVRQAPG | 1-CD3-C21-VH |
| | KGLEWVAAIWYDASKKYYADSVKGRFTISRDNSKNTLYLQM | |
| | NSLRAEDTAVYYCARSNYDVLTRYYQYGINVWGQGTTVTVSS | |
| 99 | AIQLTQSPSSLSASVGDRVTITCRASQGISSALAWYQQKPGKAP | 1-CD3-M12-VH |
| | KLLIYDASSLENGVPSRFSGSGSGTDFTLTISSLQPEDFATYYCQ | |
| | QFNSYPRTFGQGTKVEIK | |
| 100 | AIQLTQSPSSLSASVGDRVTITCRASQGISSALAWYQQKPGKAP | 1-CD3-M14-VH |
| | KLLIYDASGLENGVPSRFSGSGSGTDFTLTISSLQPEDFATYYCQ | |
| | QFKSYPRTFGQGTKVEIK | |
| 101 | AIQLTQSPSSLSASVGDRVTITCRASQGISSALAWYQQKPGKAP | 1-CD3-M15-VH |
| | KLLIYDASGLESGVPSRFSGSGSGTDFTLTISSLQPEDFATYYCQ | |
| | QFNSYPRTFGQGTKVEIK | |
| 102 | GFTFSX₂YGIH | Consensus CD3 |
| | | CDR1 VH; X₂ = R or |
| | | N |
| 103 | AIWYDX₄SKX₅YYADSVKG | Consensus CD3 |
| | | CDR2 VH; X₄ = G or |
| | | A; X₅ = K or T or D |
| 104 | SNYDILTX₈X₉YX₁₀X₁₁GX₁₂X₁₃V | Consensus CD3 |
| | | CDR3 VH; X₈ = R or |
| | | G; X₉ = Y or H; |
| | | X₁₀ = Q or S; X₁₁ = Y |
| | | or H; X₁₂ = I or L; |
| | | X₁₃ = N or D |
| 105 | DASSLEX₁ | Consensus CD3 |
| | | CDR2 VL |
| | | X₁ = R or G or E |
| 106 | QQFX₂SYPRT | Consensus CD3 |
| | | CDR3 VL |
| | | X₂ = R or N |
| 107 | AIWYDGSKKYYADSVKG | 1-CD3-VH-CDR2 |
| 108 | SNYDILTRYYQYGINV | 1,2,4-CD3-VH- |
| | | CDR3 |
| 109 | NYGIH | 2-CD3-VH-CDR1 |
| 110 | AIWYDASKKYYADSVKG | 2, 3, 4, 9, 10, 14, |
| | | 15-CD3-VH-CDR2 |
| 111 | SNYDILTRYYSHGINV | 7, 8-CD3-VH-CDR3 |
| 112 | AIWYDASKDYYADSVKG | 11, 13-CD3-VH- |
| | | CDR2 |
| 113 | SNYDILTGHYQYGINV | 16-CD3-VH-CDR3 |
| 114 | DASSLEN | 1-CD3-VL-CDR2 |
| 115 | QQFNSYPRT | 4, 7, 8, 9, 10, 11, |
| | | 12, 13, 14, 15, 16- |
| | | CD3-VL-CDR3 |
| 116 | QVQLVESGGGVVQPGX₁SLRLSCAASGFTFSX₂YGIHWVRQAP | Consensus VH |
| | GKX₃LEWVAAIWYDX₄SKX₅YYADSVKGRFTISRDNSX₆NTLX₇ | amino acid sequence |
| | LQMNSLRAEDTAVYYCARSNYDILTX₈X₉YX₁₀X₁₁GX₁₂X₁₃VWG | for CD3 |
| | QGTTVTVSS | X₁ = R or G or E |
| | | X₂ = R or N |
| | | X₃ = G or C |
| | | X₄ = G or A |
| | | X₅ = K or T or D |
| | | X₆ = K or E or S |
| | | X₇ = Y or D |
| | | X₈ = R or G |
| | | X₉ = Y or H |
| | | X₁₀ = Q or S |
| | | X₁₁ = Y or H |
| | | X₁₂ = I or L |
| | | X₁₃ =N or D |
| 117 | AIQLTQSPSSLSASVGDRVTITCRASQGISSALAWYQQKPGKAP | Consensus VL |
| | KLLIYDASSLEX₁GVPSRFSGSGSGTDFTLTISSLQPEDFATYYC | amino acid sequence |
| | QQFX₂SYPRTFGX₃GTKVEIK | for CD3 |
| | | X₁ = N or S |
| | | X₂ = K or N |
| | | X₃ = Q or C |
| 118 | SYWIG | 2, 3, 4, 6, 9, |
| | | 14-CD19-VH-CDR1 |
| 119 | IIYPGDSDTRYSPSFQG | 2, 3, 4, 6, 8, 9, |
| | | 14-CD19-VH-CDR2 |
| 120 | GGTTIFGVVFEY | 2, 14-CD19-VH- |
| | | CDR3 |
| 121 | RASQSINNYLN | 2, 14-CD19-VL- |
| | | CDR1 |
| 122 | GASSLQS | 2, 14-CD19-VL- |
| | | CDR2 |
| 123 | QQTYSTPWT | 2, 14-CD19-VL- |
| | | CDR3 |
| 124 | QSIMGVIYVAFDV | 3-CD19-VH-CDR3 |
| 125 | RASQSISSYLN | 3-CD19-VL-CDR1 |
| 126 | AASSLQS | 3, 4, 9-CD19-VL- |
| | | CDR2 |
| 127 | QQSYSNPWT | 3-CD19-VL-CDR3 |
| 128 | GGATIFGVVFDY | 4-CD19-VH-CDR3 |
| 129 | RASQSINSYLS | 4-CD19-VL-CDR1 |
| 130 | QQSYSTPPT | 4-CD19-VL-CDR3 |
| 131 | SYYWS | 5, 10-CD19-VH- |
| | | CDR1 |
| 132 | YIYYSGSTKYNPSLKS | 5-CD19-VH-CDR2 |
| 133 | GLAAAGDYYYGMDV | 5-CD19-VH-CDR3 |
| 134 | RASQGISSALA | 5-CD19-VL-CDR1 |
| 135 | QQFNSYPLT | 5-CD19-VL-CDR3 |
| 136 | QSIVATITWAFDI | 6-CD19-VH-CDR3 |
| 137 | RASQGISSYLA | 6-CD19-VL-CDR1 |
| 138 | AASTLQS | 6-CD19-VL-CDR2 |
| 139 | QQLNSYPYT | 6-CD19-VL-CDR3 |
| 140 | SGSYNWG | 7-CD19-VH-CDR1 |
| 141 | SIYYSGSTYYNPSLKS | 7-CD19-VH-CDR2 |
| 142 | HTGGFDY | 7-CD19-VH-CDR3 |
| 143 | RASQSVSSNLA | 7-CD19-VL-CDR1 |
| 144 | GASIRAT | 7-CD19-VL-CDR2 |
| 145 | QQYNNWPLT | 7-CD19-VL-CDR3 |
| 146 | SYWIA | 8-CD19-VH-CDR1 |
| 147 | HGGYNYGFTDYYYGMDV | 8-CD19-VH-CDR3 |
| 148 | RSSQRLVHSDGDTYLS | 8-CD19-VL-CDR1 |
| 149 | KISNRFS | 8-CD19-VL-CDR2 |
| 150 | MQATQFPYT | 8-CD19-VL-CDR3 |
| 151 | RSIAGTTSWSFDL | 9-CD19-VH-CDR3 |
| 152 | RTSQDIRNDLG | 9-CD19-VL-CDR1 |
| 153 | LQDYNYPLT | 9-CD19-VL-CDR3 |
| 154 | YIYYSGRTNYNPSLKS | 10-CD19-VH-CDR2 |
| 155 | HHWGSALDY | 10-CD19-VH-CDR3 |
| 156 | RSSQSLVYSDGNTYLN | 10-CD19-VL-CDR1 |
| 157 | KVSNRDS | 10-CD19-VL-CDR2 |
| 158 | MQGTHWPWT | 10-CD19-VL-CDR3 |
| 159 | QVQLVESGGGVVQPGGSLRLSCAASGFTFSRYGIHWVRQAPG | 16-CD3-VH |
| | KGLEWVAAIWYDASKTYYADSVKGRFTISRDNSKNTLYLQM | |
| | NSLRAEDTAVYYCARSNYDILTGHYQYGINVWGQGTTVTVSS | |
| 160 | GGGGS | G4S linker |
| 161 | GGGGSGGGGS | (G4S)2 linker |
| 162 | GGGGSGGGGSGGGGS | (G4S)3 linker |
| 163 | DKTHTCPPCPAPEAEGAPSVFLFPPKPKDTLMISRTPEVTCVVV | Fc-IgG1.3f-Knob |
| | DVSHEDPEVKFNWYVDGVEVHNAKTKPREEQYNSTYRVVSV | L234A, L235E, |
| | LTVLHQDWLNGKEYKCKVSNKALPAPIEKTISKAKGQPREPQV | G237A; |
| | YTLPPCREEMTKNQVSLWCLVKGFYPSDIAVEWESNGQPENN | D356E, L358M; |
| | YKTTPPVLDSDGSFFLYSKLTVDKSRWQQGNVFSCSVMHEAL | S354C, T366W |
| | HNHYTQKSLSLSPG | |
| 164 | DKTHTCPPCPAPEAEGAPSVFLFPPKPKDTLMISRTPEVTCVVV | Fc-IgG1.3f-Hole |
| | DVSHEDPEVKFNWYVDGVEVHNAKTKPREEQYNSTYRVVSV | L234A, L235E, |
| | LTVLHQDWLNGKEYKCKVSNKALPAPIEKTISKAKGQPREPQV | G237A; |
| | CTLPPSREEMTKNQVSLSCAVKGFYPSDIAVEWESNGQPENNY | D356E, L358M; |
| | KTTPPVLDSDGSFFLVSKLTVDKSRWQQGNVFSCSVMHEALH | Y349C, T366S, |
| | NHYTQKSLSLSPG | L368A, Y407V |
| 165 | DKTHTCPPCPAPEAAGGPSVFLFPPKPKDTLMISRTPEVTCVVV | Fc-IgG1 KEMA |
| | SVSHEDPEVKFNWYVDGVEVHNAKTKPREEQYNSTYRVVSVL | AAS-Knob |
| | TVLHQDWLNGKEYKCKVSNKALPAPIEKTISKAKGQPREPQV | L234A, L235A, |
| | YTLPPCREEMTKNQVSLWCLVKGFYPSDIAVEWESNGQPENN | D265S; |
| | YKTTPPVLDSDGSFFLYSKLTVDKSRWQQGNVFSCSVMHEAL | D356E, L358M; |
| | HNHYTQKSLSLSPG | S354C, T366W |
| 166 | DKTHTCPPCPAPEAAGGPSVFLFPPKPKDTLMISRTPEVTCVVV | Fc-IgG1 KEMA |
| | SVSHEDPEVKFNWYVDGVEVHNAKTKPREEQYNSTYRVVSVL | AAS-HoleCC |
| | TVLHQDWLNGKEYKCKVSNKALPAPIEKTISKAKGQPREPQVC | L234A, L235A, |
| | TLPPSREEMTKNQVSLSCAVKGFYPSDIAVEWESNGQPENNYK | D265S; |
| | TTPPVLDSDGSFFLVSKLTVDKSRWQQGNVFSCSVMHEALHN | D356E, L358M; |
| | HYTQKSLSLSPG | Y349C, T366S, |
| | | L368A, Y407V |
| 167 | ERKSCVECPPCPAPPVAGKSVFLFPPKPKDTLMISRTPEVTCVV | Fc-IgG2.3 P238K- |
| | VDVSHEDPEVQFNWYVDGVEVHNAKTKPREEQFNSTFRVVSV | Knob |
| | LTVVHQDWLNGKEYKCKVSNKGLPAPIEKTISKTKGQPREPQV | P238K; |
| | YTLPPCREEMTKNQVSLWCLVKGFYPSDIAVEWESNGQPENN | D356E, L358M; |
| | YKTTPPMLDSDGSFFLYSKLTVDKSRWQQGNVFSCSVMHEAL | S354C, T366W |
| | HNHYTQKSLSLSPG | |
| 168 | ERKSCVECPPCPAPPVAGKSVFLFPPKPKDTLMISRTPEVTCVV | Fc-IgG2.3 P238K- |
| | VDVSHEDPEVQFNWYVDGVEVHNAKTKPREEQFNSTFRVVSV | Hole |
| | LTVVHQDWLNGKEYKCKVSNKGLPAPIEKTISKTKGQPREPQV | P238K; |
| | CTLPPSREEMTKNQVSLSCAVKGFYPSDIAVEWESNGQPENNY | D356E, L358M; |
| | KTTPPMLDSDGSFFLVSKLTVDKSRWQQGNVFSCSVMHEALH | Y349C, T366S, |
| | NHYTQKSLSLSPG | L368A, Y407V |
| 169 | ASTKGPSVFPLAPSSKSTSGGTAALGCLVKDYFPEPVTVSWNS | IgG1 CH1 |
| | GALTSGVHTFPAVLQSSGLYSLSSVVTVPSSSLGTQTYICNVNH | |
| | KPSNTKVDKKVEPKSC | |
| 170 | ASTKGPSVFPLAPCSRSTSESTAALGCLVKDYFPEPVTVSWNSG | IgG2 CH1 |
| | ALTSGVHTFPAVLQSSGLYSLSSVVTVPSSNFGTQTYTCNVDH | |
| | KPSNTKVDKTV | |
| 171 | RTVAAPSVFIFPPSDEQLKSGTASVVCLLNNFYPREAKVQWKV | Constant Kappa |
| | DNALQSGNSQESVTEQDSKDSTYSLSSTLTLSKADYEKHKVYA | |
| | CEVTHQGLSSPVTKSFNRGEC | |
| 172 | QVQLQQWGAGLLKPSETLSLTCAVYGGSFSDYYWSWVRQPP | TCE001 |
| | GKCLEWIGEITHSGSTNYNPSLKSRVTISVDTSKNQFSLKLSSVT | Chain 1 |
| | AADTAVYYCARGGLDIFTGPYYFDSWGQGTLVTVSSGGGGSG | Heavy chain amino |
| | GGGSGGGGSDIQMTQSPSSLSASVGDRVTITCRASQEIRKDLA | acid sequence |
| | WYQQKPGKAPKLLIYGASSLRSGVPSRFSGSGSGTDFTLTISSL | |
| | QPEDFATYYCQQDYNYPPTFGCGTKVEIKDKTHTCPPCPAPEA | |
| | AGGPSVFLFPPKPKDTLMISRTPEVTCVVVSVSHEDPEVKFNW | |
| | YVDGVEVHNAKTKPREEQYNSTYRVVSVLTVLHQDWLNGKE | |
| | YKCKVSNKALPAPIEKTISKAKGQPREPQVCTLPPSREEMTKNQ | |
| | VSLSCAVKGFYPSDIAVEWESNGQPENNYKTTPPVLDSDGSFF | |
| | LVSKLTVDKSRWQQGNVFSCSVMHEALHNHYTQKSLSLSPG | |
| 173 | QVQLVESGGGVVQPGGSLRLSCAASGFTFSRYGIHWVRQAPG | TCE001 |
| | KCLEWVAAIWYDASKTYYADSVKGRFTISRDNSKNTLYLQMN | Heavy chain Chain |
| | SLRAEDTAVYYCARSNYDILTRYYQYGLDVWGQGTTVTVSSG | 2 |
| | GGGSGGGGSGGGGSAIQLTQSPSSLSASVGDRVTITCRASQGIS | amino acid |
| | SALAWYQQKPGKAPKLLIYDASSLESGVPSRFSGSGSGTDFTLT | sequence |
| | ISSLQPEDFATYYCQQFKSYPRTFGCGTKVEIKDKTHTCPPCPA | |
| | PEAAGGPSVFLFPPKPKDTLMISRTPEVTCVVVSVSHEDPEVKF | |
| | NWYVDGVEVHNAKTKPREEQYNSTYRVVSVLTVLHQDWLNG | |
| | KEYKCKVSNKALPAPIEKTISKAKGQPREPQVYTLPPCREEMT | |
| | KNQVSLWCLVKGFYPSDIAVEWESNGQPENNYKTTPPVLDSD | |
| | GSFFLYSKLTVDKSRWQQGNVFSCSVMHEALHNHYTQKSLSL | |
| | SPG | |
| 174 | QVQLQQWGAGLLKPSETLSLTCAVYGGSFSDYYWSWVRQPP | TCE002 |
| | GKCLEWIGEITHSGSTNYNPSLKSRVTISVDTSKNQFSLKLSSVT | Chain 1 |
| | AADTAVYYCARGGLDIFTGPYYFDSWGQGTLVTVSSGGGGSG | Heavy chain amino |
| | GGGSGGGGSDIQMTQSPSSLSASVGDRVTITCRASQEIRKDLA | acid sequence |
| | WYQQKPGKAPKLLIYGASSLRSGVPSRFSGSGSGTDFTLTISSL | |
| | QPEDFATYYCQQDYNYPPTFGCGTKVEIKDKTHTCPPCPAPEA | |
| | AGGPSVFLFPPKPKDTLMISRTPEVTCVVVSVSHEDPEVKFNW | |
| | YVDGVEVHNAKTKPREEQYNSTYRVVSVLTVLHQDWLNGKE | |
| | YKCKVSNKALPAPIEKTISKAKGQPREPQVCTLPPSREEMTKNQ | |
| | VSLSCAVKGFYPSDIAVEWESNGQPENNYKTTPPVLDSDGSFF | |
| | LVSKLTVDKSRWQQGNVFSCSVMHEALHNHYTQKSLSLSPG | |
| 175 | QVQLVESGGGVVQPGGSLRLSCAASGFTFSRYGIHWVRQAPG | TCE002 |
| | KGLEWVAAIWYDASKTYYADSVKGRFTISRDNSKNTLYLQMN | Heavy chain Chain |
| | SLRAEDTAVYYCARSNYDILTRYYQYGLDVWGQGTTVTVSSA | 2 |
| | STKGPSVFPLAPSSKSTSGGTAALGCLVKDYFPEPVTVSWNSG | amino acid |
| | ALTSGVHTFPAVLQSSGLYSLSSVVTVPSSSLGTQTYICNVNHK | sequence |
| | PSNTKVDKKVEPKSCDKTHTCPPCPAPEAAGGPSVFLFPPKPKD | |
| | TLMISRTPEVTCVVVSVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPCREEMTKNQVSLWCLVKGFYPSD | |
| | IAVEWESNGQPENNYKTTPPVLDSDGSFFLYSKLTVDKSRWQQ | |
| | GNVFSCSVMHEALHNHYTQKSLSLSPG | |
| 176 | AIQLTQSPSSLSASVGDRVTITCRASQGISSALAWYQQKPGKAP | TCE002 |
| | KLLIYDASSLESGVPSRFSGSGSGTDFTLTISSLQPEDFATYYCQ | Light chain Chain 3 |
| | QFKSYPRTFGQGTKVEIKRTVAAPSVFIFPPSDEQLKSGTASVV | amino acid |
| | CLLNNFYPREAKVQWKVDNALQSGNSQESVTEQDSKDSTYSL | sequence |
| | SSTLTLSKADYEKHKVYACEVTHQGLSSPVTKSFNRGEC | |
| 177 | QVQLQQWGAGLLKPSETLSLTCAVYGGSFSDYYWSWVRQPP | TCE003 |
| | GKCLEWIGEITHSGSTNYNPSLKSRVTISVDTSKNQFSLKLSSVT | Chain 1 |
| | AADTAVYYCARGGLDIFTGPYYFDSWGQGTLVTVSSGGGGSG | Heavy chain amino |
| | GGGSGGGGSDIQMTQSPSSLSASVGDRVTITCRASQEIRKDLA | acid sequence |
| | WYQQKPGKAPKLLIYGASSLRSGVPSRFSGSGSGTDFTLTISSL | |
| | QPEDFATYYCQQDYNYPPTFGCGTKVEIKDKTHTCPPCPAPEA | |
| | AGGPSVFLFPPKPKDTLMISRTPEVTCVVVSVSHEDPEVKFNW | |
| | YVDGVEVHNAKTKPREEQYNSTYRVVSVLTVLHQDWLNGKE | |
| | YKCKVSNKALPAPIEKTISKAKGQPREPQVCTLPPSREEMTKNQ | |
| | VSLSCAVKGFYPSDIAVEWESNGQPENNYKTTPPVLDSDGSFF | |
| | LVSKLTVDKSRWQQGNVFSCSVMHEALHNHYTQKSLSLSPG | |
| 178 | QVQLVESGGGVVQPGGSLRLSCAASGFTFSRYGIHWVRQAPG | TCE003 |
| | KCLEWVAAIWYDASKTYYADSVKGRFTISRDNSKNTLYLQMN | Heavy chain Chain |
| | SLRAEDTAVYYCARSNYDILTRYYSHGINVWGQGTTVTVSSG | 2 |
| | GGGSGGGGSGGGGSAIQLTQSPSSLSASVGDRVTITCRASQGIS | amino acid |
| | SALAWYQQKPGKAPKLLIYDASSLESGVPSRFSGSGSGTDFTLT | sequence |
| | ISSLQPEDFATYYCQQFNSYPRTFGCGTKVEIKDKTHTCPPCPA | |
| | PEAAGGPSVFLFPPKPKDTLMISRTPEVTCVVVSVSHEDPEVKF | |
| | NWYVDGVEVHNAKTKPREEQYNSTYRVVSVLTVLHQDWLNG | |
| | KEYKCKVSNKALPAPIEKTISKAKGQPREPQVYTLPPCREEMT | |
| | KNQVSLWCLVKGFYPSDIAVEWESNGQPENNYKTTPPVLDSD | |
| | GSFFLYSKLTVDKSRWQQGNVFSCSVMHEALHNHYTQKSLSL | |
| | SPG | |
| 179 | QVQLQQWGAGLLKPSETLSLTCAVYGGSFSDYYWSWVRQPP | TCE004 |
| | GKCLEWIGEITHSGSTNYNPSLKSRVTISVDTSKNQFSLKLSSVT | Chain 1 |
| | AADTAVYYCARGGLDIFTGPYYFDSWGQGTLVTVSSGGGGSG | Heavy chain amino |
| | GGGSGGGGSDIQMTQSPSSLSASVGDRVTITCRASQEIRKDLA | acid sequence |
| | WYQQKPGKAPKLLIYGASSLRSGVPSRFSGSGSGTDFTLTISSL | |
| | QPEDFATYYCQQDYNYPPTFGCGTKVEIKDKTHTCPPCPAPEA | |
| | AGGPSVFLFPPKPKDTLMISRTPEVTCVVVSVSHEDPEVKFNW | |
| | YVDGVEVHNAKTKPREEQYNSTYRVVSVLTVLHQDWLNGKE | |
| | YKCKVSNKALPAPIEKTISKAKGQPREPQVCTLPPSREEMTKNQ | |
| | VSLSCAVKGFYPSDIAVEWESNGQPENNYKTTPPVLDSDGSFF | |
| | LVSKLTVDKSRWQQGNVFSCSVMHEALHNHYTQKSLSLSPG | |
| 180 | QVQLVESGGGVVQPGGSLRLSCAASGFTFSRYGIHWVRQAPG | TCE004 |
| | KGLEWVAAIWYDASKTYYADSVKGRFTISRDNSKNTLYLQMN | Heavy chain Chain |
| | SLRAEDTAVYYCARSNYDILTRYYSHGINVWGQGTTVTVSSAS | 2 |
| | TKGPSVFPLAPSSKSTSGGTAALGCLVKDYFPEPVTVSWNSGA | amino acid |
| | LTSGVHTFPAVLQSSGLYSLSSVVTVPSSSLGTQTYICNVNHKP | sequence |
| | SNTKVDKKVEPKSCDKTHTCPPCPAPEAAGGPSVFLFPPKPKD | |
| | TLMISRTPEVTCVVVSVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPCREEMTKNQVSLWCLVKGFYPSD | |
| | IAVEWESNGQPENNYKTTPPVLDSDGSFFLYSKLTVDKSRWQQ | |
| | GNVFSCSVMHEALHNHYTQKSLSLSPG | |
| 181 | AIQLTQSPSSLSASVGDRVTITCRASQGISSALAWYQQKPGKAP | TCE004 |
| | KLLIYDASSLESGVPSRFSGSGSGTDFTLTISSLQPEDFATYYCQ | Light chain Chain 3 |
| | QFNSYPRTFGQGTKVEIKRTVAAPSVFIFPPSDEQLKSGTASVV | amino acid |
| | CLLNNFYPREAKVQWKVDNALQSGNSQESVTEQDSKDSTYSL | sequence |
| | SSTLTLSKADYEKHKVYACEVTHQGLSSPVTKSFNRGEC | |
| 182 | EVQLVQSGAEVKKPGESLKISCKGSGYSFTSYWIGWVRQMPG | TCE005 |
| | KCLEWMGIIYPGDSDTRYSPSFQGQVTISADKSISTAYLQWSSL | Chain 1 |
| | KASDTAMYYCARGGTTIFGVVFEYWGQGTLVTVSSGGGGSGG | Heavy chain amino |
| | GGSGGGGSDIQMTQSPSSLSASVGDRVTITCRASQSINNYLNW | acid sequence |
| | YQQKPGKAPKLLIYGASSLQSGVPSRFSGSGSGTDFTLTISSLQP | |
| | EDFATYYCQQTYSTPWTFGCGTKVEIKDKTHTCPPCPAPEAAG | |
| | GPSVFLFPPKPKDTLMISRTPEVTCVVVSVSHEDPEVKFNWYV | |
| | DGVEVHNAKTKPREEQYNSTYRVVSVLTVLHQDWLNGKEYK | |
| | CKVSNKALPAPIEKTISKAKGQPREPQVCTLPPSREEMTKNQVS | |
| | LSCAVKGFYPSDIAVEWESNGQPENNYKTTPPVLDSDGSFFLV | |
| | SKLTVDKSRWQQGNVFSCSVMHEALHNHYTQKSLSLSPG | |
| 183 | QVQLVESGGGVVQPGGSLRLSCAASGFTFSRYGIHWVRQAPG | TCE005 |
| | KCLEWVAAIWYDASKTYYADSVKGRFTISRDNSKNTLYLQMN | Heavy chain Chain |
| | SLRAEDTAVYYCARSNYDILTRYYQYGLDVWGQGTTVTVSSG | 2 |
| | GGGSGGGGSGGGGSAIQLTQSPSSLSASVGDRVTITCRASQGIS | amino acid |
| | SALAWYQQKPGKAPKLLIYDASSLESGVPSRFSGSGSGTDFTLT | sequence |
| | ISSLQPEDFATYYCQQFKSYPRTFGCGTKVEIKDKTHTCPPCPA | |
| | PEAAGGPSVFLFPPKPKDTLMISRTPEVTCVVVSVSHEDPEVKF | |
| | NWYVDGVEVHNAKTKPREEQYNSTYRVVSVLTVLHQDWLNG | |
| | KEYKCKVSNKALPAPIEKTISKAKGQPREPQVYTLPPCREEMT | |
| | KNQVSLWCLVKGFYPSDIAVEWESNGQPENNYKTTPPVLDSD | |
| | GSFFLYSKLTVDKSRWQQGNVFSCSVMHEALHNHYTQKSLSL | |
| | SPG | |
| 184 | EVQLVQSGAEVKKPGESLKISCKGSGYSFTSYWIGWVRQMPG | TCE006 |
| | KCLEWMGIIYPGDSDTRYSPSFQGQVTISADKSISTAYLQWSSL | Chain 1 |
| | KASDTAMYYCARGGTTIFGVVFEYWGQGTLVTVSSGGGGSGG | Heavy chain amino |
| | GGSGGGGSDIQMTQSPSSLSASVGDRVTITCRASQSINNYLNW | acid sequence |
| | YQQKPGKAPKLLIYGASSLQSGVPSRFSGSGSGTDFTLTISSLQP | |
| | EDFATYYCQQTYSTPWTFGCGTKVEIKDKTHTCPPCPAPEAAG | |
| | GPSVFLFPPKPKDTLMISRTPEVTCVVVSVSHEDPEVKFNWYV | |
| | DGVEVHNAKTKPREEQYNSTYRVVSVLTVLHQDWLNGKEYK | |
| | CKVSNKALPAPIEKTISKAKGQPREPQVCTLPPSREEMTKNQVS | |
| | LSCAVKGFYPSDIAVEWESNGQPENNYKTTPPVLDSDGSFFLV | |
| | SKLTVDKSRWQQGNVFSCSVMHEALHNHYTQKSLSLSPG | |
| 185 | QVQLVESGGGVVQPGGSLRLSCAASGFTFSRYGIHWVRQAPG | TCE006 |
| | KGLEWVAAIWYDASKTYYADSVKGRFTISRDNSKNTLYLQMN | Heavy chain Chain |
| | SLRAEDTAVYYCARSNYDILTRYYQYGLDVWGQGTTVTVSSA | 2 |
| | STKGPSVFPLAPSSKSTSGGTAALGCLVKDYFPEPVTVSWNSG | amino acid |
| | ALTSGVHTFPAVLQSSGLYSLSSVVTVPSSSLGTQTYICNVNHK | sequence |
| | PSNTKVDKKVEPKSCDKTHTCPPCPAPEAAGGPSVFLFPPKPKD | |
| | TLMISRTPEVTCVVVSVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPCREEMTKNQVSLWCLVKGFYPSD | |
| | IAVEWESNGQPENNYKTTPPVLDSDGSFFLYSKLTVDKSRWQQ | |
| | GNVFSCSVMHEALHNHYTQKSLSLSPG | |
| 186 | AIQLTQSPSSLSASVGDRVTITCRASQGISSALAWYQQKPGKAP | TCE006 |
| | KLLIYDASSLESGVPSRFSGSGSGTDFTLTISSLQPEDFATYYCQ | Light chain Chain 3 |
| | QFKSYPRTFGQGTKVEIKRTVAAPSVFIFPPSDEQLKSGTASVV | amino acid |
| | CLLNNFYPREAKVQWKVDNALQSGNSQESVTEQDSKDSTYSL | sequence |
| | SSTLTLSKADYEKHKVYACEVTHQGLSSPVTKSFNRGEC | |
| 187 | EVQLVQSGAEVKKPGESLKISCKGSGYSFTSYWIGWVRQMPG | TCE007 |
| | KCLEWMGIIYPGDSDTRYSPSFQGQVTISADKSISTAYLQWSSL | Chain 1 |
| | KASDTAMYYCARGGTTIFGVVFEYWGQGTLVTVSSGGGGSGG | Heavy chain amino |
| | GGSGGGGSDIQMTQSPSSLSASVGDRVTITCRASQSINNYLNW | acid sequence |
| | YQQKPGKAPKLLIYGASSLQSGVPSRFSGSGSGTDFTLTISSLQP | |
| | EDFATYYCQQTYSTPWTFGCGTKVEIKDKTHTCPPCPAPEAAG | |
| | GPSVFLFPPKPKDTLMISRTPEVTCVVVSVSHEDPEVKFNWYV | |
| | DGVEVHNAKTKPREEQYNSTYRVVSVLTVLHQDWLNGKEYK | |
| | CKVSNKALPAPIEKTISKAKGQPREPQVCTLPPSREEMTKNQVS | |
| | LSCAVKGFYPSDIAVEWESNGQPENNYKTTPPVLDSDGSFFLV | |
| | SKLTVDKSRWQQGNVFSCSVMHEALHNHYTQKSLSLSPG | |
| 188 | QVQLVESGGGVVQPGGSLRLSCAASGFTFSRYGIHWVRQAPG | TCE007 |
| | KCLEWVAAIWYDASKTYYADSVKGRFTISRDNSKNTLYLQMN | Heavy chain Chain |
| | SLRAEDTAVYYCARSNYDILTRYYSHGINVWGQGTTVTVSSG | 2 |
| | GGGSGGGGSGGGGSAIQLTQSPSSLSASVGDRVTITCRASQGIS | amino acid |
| | SALAWYQQKPGKAPKLLIYDASSLESGVPSRFSGSGSGTDFTLT | sequence |
| | ISSLQPEDFATYYCQQFNSYPRTFGCGTKVEIKDKTHTCPPCPA | |
| | PEAAGGPSVFLFPPKPKDTLMISRTPEVTCVVVSVSHEDPEVKF | |
| | NWYVDGVEVHNAKTKPREEQYNSTYRVVSVLTVLHQDWLNG | |
| | KEYKCKVSNKALPAPIEKTISKAKGQPREPQVYTLPPCREEMT | |
| | KNQVSLWCLVKGFYPSDIAVEWESNGQPENNYKTTPPVLDSD | |
| | GSFFLYSKLTVDKSRWQQGNVFSCSVMHEALHNHYTQKSLSL | |
| | SPG | |
| 189 | EVQLVQSGAEVKKPGESLKISCKGSGYSFTSYWIGWVRQMPG | TCE008 |
| | KCLEWMGIIYPGDSDTRYSPSFQGQVTISADKSISTAYLQWSSL | Chain 1 |
| | KASDTAMYYCARGGTTIFGVVFEYWGQGTLVTVSSGGGGSGG | Heavy chain amino |
| | GGSGGGGSDIQMTQSPSSLSASVGDRVTITCRASQSINNYLNW | acid sequence |
| | YQQKPGKAPKLLIYGASSLQSGVPSRFSGSGSGTDFTLTISSLQP | |
| | EDFATYYCQQTYSTPWTFGCGTKVEIKDKTHTCPPCPAPEAAG | |
| | GPSVFLFPPKPKDTLMISRTPEVTCVVVSVSHEDPEVKFNWYV | |
| | DGVEVHNAKTKPREEQYNSTYRVVSVLTVLHQDWLNGKEYK | |
| | CKVSNKALPAPIEKTISKAKGQPREPQVCTLPPSREEMTKNQVS | |
| | LSCAVKGFYPSDIAVEWESNGQPENNYKTTPPVLDSDGSFFLV | |
| | SKLTVDKSRWQQGNVFSCSVMHEALHNHYTQKSLSLSPG | |
| 190 | QVQLVESGGGVVQPGGSLRLSCAASGFTFSRYGIHWVRQAPG | TCE008 |
| | KGLEWVAAIWYDASKTYYADSVKGRFTISRDNSKNTLYLQMN | Heavy chain Chain |
| | SLRAEDTAVYYCARSNYDILTRYYSHGINVWGQGTTVTVSSAS | 2 |
| | TKGPSVFPLAPSSKSTSGGTAALGCLVKDYFPEPVTVSWNSGA | amino acid |
| | LTSGVHTFPAVLQSSGLYSLSSVVTVPSSSLGTQTYICNVNHKP | sequence |
| | SNTKVDKKVEPKSCDKTHTCPPCPAPEAAGGPSVFLFPPKPKD | |
| | TLMISRTPEVTCVVVSVSHEDPEVKFNWYVDGVEVHNAKTKP | |
| | REEQYNSTYRVVSVLTVLHQDWLNGKEYKCKVSNKALPAPIE | |
| | KTISKAKGQPREPQVYTLPPCREEMTKNQVSLWCLVKGFYPSD | |
| | IAVEWESNGQPENNYKTTPPVLDSDGSFFLYSKLTVDKSRWQQ | |
| | GNVFSCSVMHEALHNHYTQKSLSLSPG | |
| 191 | AIQLTQSPSSLSASVGDRVTITCRASQGISSALAWYQQKPGKAP | TCE008 |
| | KLLIYDASSLESGVPSRFSGSGSGTDFTLTISSLQPEDFATYYCQ | Light chain Chain 3 |
| | QFNSYPRTFGQGTKVEIKRTVAAPSVFIFPPSDEQLKSGTASVV | amino acid |
| | CLLNNFYPREAKVQWKVDNALQSGNSQESVTEQDSKDSTYSL | sequence |
| | SSTLTLSKADYEKHKVYACEVTHQGLSSPVTKSFNRGEC | |
| 192 | QVQLVESGGGVVQPGX₁SLRLSCAASGFTFSX₂YGIHWVRQAP | Consensus VH |
| | GKX₃LEWVAAIWYDASKX₄YYADSVKGRFTISRDNSX₅NTLYL | amino acid sequence |
| | QMNSLRAEDTAVYYCARSNYDILTX₆X₇YX₈X₉GX₁₀X₁₁VWGQG | X₁ = R or G or E |
| | TTVTVSS | X₂ = R or N |
| | | X₃ = G or C |
| | | X₄ = K or T or D |
| | | X₅ = K or E or S |
| | | X₆ = R or G |
| | | X₇ = Y or H |
| | | X₈ = Q or S |
| | | X₉ = Y or H |
| | | X₁₀ = I or L |
| | | X₁₁ = N or D |
| 193 | AIQLTQSPSSLSASVGDRVTITCRASQGISSALAWYQQKPGKAP | Consensus VL |
| | KLLIYDASSLESGVPSRFSGSGSGTDFTLTISSLQPEDFATYYCQ | amino acid sequence |
| | QFX₁SYPRTFGX₂GTKVEIK | X₁ = K or N |
| | | X₂ = Q or C |
| 194 | AIWYDASKX₄YYADSVKG | CDR-H2 Consensus |
| | | amino acid sequence |
| | | X₄ = K or T or D |
| 195 | QVQLVESGGGVVQPGESLRLSCAASGFTFSRYGIHWVRQAPG | 10-CD3-VH |
| | KGLEWVAAIWYDASKKYYADSVKGRFTISRDNSKNTLYLQM | |
| | NSLRAEDTAVYYCARSNYDILTRYYQYGLDVWGQGTTVTVSS | |
| 196 | QVQLVESGGGVVQPGX₁SLRLSCAASGFTFSX₂X₃GIHWVRQAP | Consensus CD3 VH |
| | GKX₄LEWVAAIWYDX₅SX₆X₇YYADSVKGRFTISRDNSX₈NTLX₉ | (all sequences) |
| | LQMNSLRAEDTAVYYCARSNX₁₀DX₁₁LTX₁₂X₁₃YX₁₄X₁₅GX₁₆X₁₇ | amino acid sequence |
| | VWGQGTTVTVSS | X₁ = R or G or E |
| | | X₂ = R or N |
| | | X₃ = Y or H |
| | | X₄ = G or C |
| | | X₅ = G or A |
| | | X₆ = K or H or N |
| | | X₇ = K or T or D |
| | | X8 = K or E or S |
| | | X9 = Y or D |
| | | X₁₀ = Y or F |
| | | X₁₁ = I or V |
| | | X₁₂ = R or G |
| | | X₁₃ = Y or H |
| | | X₁₄ = Q or S |
| | | X₁₅ = Y or H |
| | | X₁₆ = I or L |
| | | X₁₇ = N or D |
| 197 | AIQLTQSPSSLSASVGDRVTITCRASQGISSALAWYQQKPGKAP | Consensus CD3 VL |
| | KLLIYDASX₁LEX₂GVPSRFSGSGSGTDFTLTISSLQPEDFATYYC | (all sequences) |
| | QQFX₃SYPRTFGX₄GTKVEIK | amino acid sequence |
| | | X₁ = S or G |
| | | X₂ = N or S |
| | | X₃ = K or N |
| | | X₄ = Q or C |
| 198 | X₁X₂GIH | CD3 VH CDR1 |
| | | consensus (all |
| | | sequences) amino |
| | | acid sequence |
| | | X₁ = R or N |
| | | X₂ = Y or H |
| 199 | AIWYDX₁SX₂X₃YYADSVKG | CD3 VH CDR2 |
| | | consensus (all |
| | | sequences) amino |
| | | acid sequence |
| | | X₁ = G or A |
| | | X₂ = K or H or N |
| | | X₃ = K or T or D |
| 200 | SNX₁DX₂LTX₃X₄YX₅X₆GX₇X₈V | CD3 VH CDR3 |
| | | consensus (all |
| | | sequences) amino |
| | | acid sequence |
| | | X₁ = Y or F |
| | | X₂ = I or V |
| | | X₃ = R or G |
| | | X₄ = Y or H |
| | | X₅ = Q or S |
| | | X₆ = Y or H |
| | | X₇ = I or L |
| | | X₈ = N or D |
| 201 | DASX₁LEX₂ | CD3 VL CDR2 |
| | | consensus (all |
| | | sequences) amino |
| | | acid sequence |
| | | X₁ = S or G |
| | | X₂ = N or S |
| 202 | NHGIH | 1-CD3-M17 VH |
| | | CDR1 |
| 203 | RHGIH | 1-CD3-M18 VH |
| | | CDR1 |
| 204 | AIWYDSSKKYYADSVKG | 1-CD3-M2 VH |
| | | CDR2 |
| 205 | AIWYDGSHKYYADSVKG | 1-CD3-M5 VH |
| | | CDR2 |
| 206 | AIWYDGSNKYYADSVKG | 1-CD3-M6 VH |
| | | CDR2 |
| 207 | AIWYDASHKYYADSVKG | 1-CD3-C5 VH |
| | | CDR2 |
| 208 | AIWYDASNKYYADSVKG | 1-CD3-C6 VH |
| | | CDR2 |
| 209 | SNYDILTRHYQYGINV | 1-CD3-M7 VH |
| | | CDR3 |
| 210 | SNYDILTGYYQYGINV | 1-CD3-M9 VH |
| | | CDR3 |
| 211 | SNYDILTRYYQHGINV | 1-CD3-M10 VH |
| | | CDR3 |
| 212 | SNFDILTRYYQYGINV | 1-CD3-M19 VH |
| | | CDR3 |
| 213 | SNFDVLTRYYQYGINV | 1-CD3-M20 VH |
| | | CDR3 |
| 214 | SNYDVLTRYYQYGINV | 1-CD3-M21 VH |
| | | CDR3 |
| 215 | DASGLEN | 1-CD3-M14 VL |
| | | CDR2 |
| 216 | DASGLES | 1-CD3-M15 VL |
| | | CDR2 |
| 217 | GGS | 3GS linker |
| 218 | GSTSGSGKPGSGEGSTKG | Linker |
| 219 | SRGGGGSGGGGSGGGGSLEMA | Linker |
| 220 | GSRGGGGSGGGGSGGGGSLEMA | linker |
| 221 | ASTKGPSVFPLAPSSKSTSGGTAWLGCEVTDYFPEPVTVSWNS | CHI region |
| | GALTSGVHTFPAVLESSGLYSLSSVVTVPSSSLGTQTYICNVNH | |
| | KPSNTKVDKKV | |
| 222 | HHHHHHH | 6 HIS Tag |
| 223 | ASTKGPSVFPLAPSSKSTSGGTAALGCLVKDYFPEPVTVSWNS | CH1 region |
| | GALTSGVHTFPAVLKSSGLYSLSSVVTVPSSSLGTQTYICNVNH | |
| | KPSNTKVDKKV | |
| 224 | QPKANPTVTLFPPSSEELQANKATLVCLISDFYPGAVTVAWKA | Lambda constant |
| | DGSPVKAGVETTKPSKQSNNKYAASSYLSLTPEQWKSHRSYSC | light |
| | QVTHEGSTVEKTVAPTECS | |
| 225 | TVAAPSVFIFPPSDEQLKSGTASVVCLLNNFYPREAKVQWKVD | Kappa constant light |
| | NALQSGNSQESVTEQDSKDSTYSLSSTLTLSKADYEKHKVYAC | |
| | EVTHQGLSSPVTKSFNRGEC | |
| 226 | EPKSCDKTHTCPPCPAPELLGGPSVFLFPPKPKDTLMISRTPEVT | wildtype IgG1 Fc |
| | CVVVDVSHEDPEVKFNWYVDGVEVHNAKTKPREEQYNSTYR | |
| | VVSVLTVLHQDWLNGKEYKCKVSNKALPAPIEKTISKAKGQP | |
| | REPQVYTLPPSREEMTKNQVSLTCLVKGFYPSDIAVEWESNGQ | |
| | PENNYKTTPPVLDSDGSFFLYSKLTVDKSRWQQGNVFSCSVM | |
| | HEALHNHYTQKSLSLSPG | |
| 227 | EPKSCDKTHTCPPCPAPELLGGPSVFLFPPKPKDTLMISRTPEVT | wildtype IgG1 Fc |
| | CVVVDVSHEDPEVKFNWYVDGVEVHNAKTKPREEQYNSTYR | |
| | VVSVLTVLHQDWLNGKEYKCKVSNKALPAPIEKTISKAKGQP | |
| | REPQVYTLPPSRDELTKNQVSLTCLVKGFYPSDIAVEWESNGQ | |
| | PENNYKTTPPVLDSDGSFFLYSKLTVDKSRWQQGNVFSCSVM | |
| | HEALHNHYTQKSLSLSPG | |
| 228 | EPKSCDKTHTCPPCPAPELLGGPSVFLFPPKPKDTLMISRTPEVT | heterodimeric Fc |
| | CVVVDVSHEDPEVKFNWYVDGVEVHNAKTKPREEQYNSTYR | polypeptide 1 |
| | VVSVLTVLHQDWLNGKEYKCKVSNKALPAPIEKTISKAKGQP | (T350V, L351Y, |
| | REPQVYVYPPSREEMTKNQVSLTCLVKGFYPSDIAVEWESNGQ | F405A and Y407V) |
| | PENNYKTTPPVLDSDGSFALVSKLTVDKSRWQQGNVFSCSVM | without effector |
| | HEALHNHYTQKSLSLSPG | mutations |
| 229 | EPKSCDKTHTCPPCPAPELLGGPSVFLFPPKPKDTLMISRTPEVT | heterodimeric Fc |
| | CVVVDVSHEDPEVKFNWYVDGVEVHNAKTKPREEQYNSTYR | polypeptide 2 |
| | VVSVLTVLHQDWLNGKEYKCKVSNKALPAPIEKTISKAKGQP | without effector |
| | REPQVYVLPPSREEMTKNQVSLLCLVKGFYPSDIAVEWESNGQ | mutations |
| | PENNYLTWPPVLDSDGSFFLYSKLTVDKSRWQQGNVFSCSVM | |
| | HEALHNHYTQKSLSLSPG | |
| 230 | EPKSCDKTHTCPPCPAPEAAGGPSVFLFPPKPKDTLMISRTPEVT | heterodimeric Fc |
| | CVVVSVSHEDPEVKFNWYVDGVEVHNAKTKPREEQYNSTYR | polypeptide 1 |
| | VVSVLTVLHQDWLNGKEYKCKVSNKALPAPIEKTISKAKGQP | (T350V, L351Y, |
| | REPQVYVYPPSREEMTKNQVSLTCLVKGFYPSDIAVEWESNGQ | F405A and Y407V)) |
| | PENNYKTTPPVLDSDGSFALVSKLTVDKSRWQQGNVFSCSVM | with LALA effector |
| | HEALHNHYTQKSLSLSPG | mutations and |
| | | D265S |
| 231 | EPKSCDKTHTCPPCPAPEAAGGPSVFLFPPKPKDTLMISRTPEVT | heterodimeric Fc |
| | CVVVSVSHEDPEVKFNWYVDGVEVHNAKTKPREEQYNSTYR | polypeptide 2 |
| | VVSVLTVLHQDWLNGKEYKCKVSNKALPAPIEKTISKAKGQP | (T350V, T366L, |
| | REPQVYVLPPSREEMTKNQVSLLCLVKGFYPSDIAVEWESNGQ | K392L and T394W) |
| | PENNYLTWPPVLDSDGSFFLYSKLTVDKSRWQQGNVFSCSVM | with LALA effector |
| | HEALHNHYTQKSLSLSPG | mutations and |
| | | D265S |
| 232 | EPKSCDKTHTCPPCPAPEAAGGPSVFLFPPKPKDTLMISRTPEVT | heterodimeric Fc |
| | CVVVDVSHEDPEVKFNWYVDGVEVHNAKTKPREEQYNSTYR | polypeptide 1 |
| | VVSVLTVLHQDWLNGKEYKCKVSNKALGAPIEKTISKAKGQP | (T350V, L351Y, |
| | REPQVYVYPPSREEMTKNQVSLTCLVKGFYPSDIAVEWESNGQ | F405A and Y407V)) |
| | PENNYKTTPPVLDSDGSFALVSKLTVDKSRWQQGNVFSCSVM | with LALA effector |
| | HEALHNHYTQKSLSLSPG | mutations and |
| | | P329G |
| 233 | EPKSCDKTHTCPPCPAPEAAGGPSVFLFPPKPKDTLMISRTPEVT | heterodimeric Fc |
| | CVVVDVSHEDPEVKFNWYVDGVEVHNAKTKPREEQYNSTYR | polypeptide 2 |
| | VVSVLTVLHQDWLNGKEYKCKVSNKALGAPIEKTISKAKGQP | (T350V, T366L, |
| | REPQVYVLPPSREEMTKNQVSLLCLVKGFYPSDIAVEWESNGQ | K392L and T394W) |
| | PENNYLTWPPVLDSDGSFFLYSKLTVDKSRWQQGNVFSCSVM | with LALA effector |
| | HEALHNHYTQKSLSLSPG | mutations and |
| | | P329G |
| 234 | QVQLQQWGAGLLKPSETLSLTCAVYGGSFSDYYWSWVR | TCE009 |
| | QPPGKCLEWIGEITHSGSTNYNPSLKSRVTISVDTSKNQFS | Chain 1 |
| | LKLSSVTAADTAVYYCARGGLDIFTGPYYFDSWGQGTLV | Heavy chain amino |
| | TVSSGGGGSGGGGSGGGGSDIQMTQSPSSLSASVGDRVTI | acid sequence |
| | TCRASQEIRKDLAWYQQKPGKAPKLLIYGASSLRSGVPSR | |
| | FSGSGSGTDFTLTISSLQPEDFATYYCQQDYNYPPTFGCG | |
| | TKVEIKERKSCVECPPCPAPPVAGKSVFLFPPKPKDTLMIS | |
| | RTPEVTCVVVDVSHEDPEVQFNWYVDGVEVHNAKTKPR | |
| | EEQFNSTFRVVSVLTVVHQDWLNGKEYKCKVSNKGLPA | |
| | PIEKTISKTKGQPREPQVCTLPPSREEMTKNQVSLSCAVK | |
| | GFYPSDIAVEWESNGQPENNYKTTPPMLDSDGSFFLVSKL | |
| | TVDKSRWQQGNVFSCSVMHEALHNHYTQKSLSLSPG | |
| 235 | QVQLVESGGGVVQPGGSLRLSCAASGFTFSRYGIHWVRQ | TCE009 |
| | APGKGLEWVAAIWYDASKTYYADSVKGRFTISRDNSKN | Heavy chain |
| | TLYLQMNSLRAEDTAVYYCARSNYDILTRYYQYGLDVW | Chain 2 amino acid |
| | GQGTTVTVSSASTKGPSVFPLAPCSRSTSESTAALGCLVK | sequence |
| | DYFPEPVTVSWNSGALTSGVHTFPAVLQSSGLYSLSSVVT | |
| | VPSSNFGTQTYTCNVDHKPSNTKVDKTVERKSCVECPPC | |
| | PAPPVAGKSVFLFPPKPKDTLMISRTPEVTCVVVDVSHED | |
| | PEVQFNWYVDGVEVHNAKTKPREEQFNSTFRVVSVLTV | |
| | VHQDWLNGKEYKCKVSNKGLPAPIEKTISKTKGQPREPQ | |
| | VYTLPPCREEMTKNQVSLWCLVKGFYPSDIAVEWESNGQ | |
| | PENNYKTTPPMLDSDGSFFLYSKLTVDKSRWQQGNVFSC | |
| | SVMHEALHNHYTQKSLSLSPG | |
| 236 | AIQLTQSPSSLSASVGDRVTITCRASQGISSALAWYQQKP | TCE009 |
| | GKAPKLLIYDASSLESGVPSRFSGSGSGTDFTLTISSLQPED | Light chain |
| | FATYYCQQFKSYPRTFGQGTKVEIKRTVAAPSVFIFPPSDE | Chain 3 amino acid |
| | QLKSGTASVVCLLNNFYPREAKVQWKVDNALQSGNSQE | sequence |
| | SVTEQDSKDSTYSLSSTLTLSKADYEKHKVYACEVTHQG | |
| | LSSPVTKSFNRGEC | |
| 237 | QVQLQQWGAGLLKPSETLSLTCAVYGGSFSDYYWSWVR | TCE010 |
| | QPPGKCLEWIGEITHSGSTNYNPSLKSRVTISVDTSKNQFS | Chain 1 |
| | LKLSSVTAADTAVYYCARGGLDIFTGPYYFDSWGQGTLV | Heavy chain amino |
| | TVSSGGGGSGGGGSGGGGSDIQMTQSPSSLSASVGDRVTI | acid sequence |
| | TCRASQEIRKDLAWYQQKPGKAPKLLIYGASSLRSGVPSR | |
| | FSGSGSGTDFTLTISSLQPEDFATYYCQQDYNYPPTFGCG | |
| | TKVEIKERKSCVECPPCPAPPVAGKSVFLFPPKPKDTLMIS | |
| | RTPEVTCVVVDVSHEDPEVQFNWYVDGVEVHNAKTKPR | |
| | EEQFNSTFRVVSVLTVVHQDWLNGKEYKCKVSNKGLPA | |
| | PIEKTISKTKGQPREPQVCTLPPSREEMTKNQVSLSCAVK | |
| | GFYPSDIAVEWESNGQPENNYKTTPPMLDSDGSFFLVSKL | |
| | TVDKSRWQQGNVFSCSVMHEALHNHYTQKSLSLSPG | |
| 238 | QVQLVESGGGVVQPGGSLRLSCAASGFTFSRYGIHWVRQ | TCE010 |
| | APGKGLEWVAAIWYDASKTYYADSVKGRFTISRDNSKN | Heavy chain |
| | TLYLQMNSLRAEDTAVYYCARSNYDILTRYYSHGINVW | Chain 2 amino acid |
| | GQGTTVTVSSASTKGPSVFPLAPCSRSTSESTAALGCLVK | sequence |
| | DYFPEPVTVSWNSGALTSGVHTFPAVLQSSGLYSLSSVVT | |
| | VPSSNFGTQTYTCNVDHKPSNTKVDKTVERKSCVECPPC | |
| | PAPPVAGKSVFLFPPKPKDTLMISRTPEVTCVVVDVSHED | |
| | PEVQFNWYVDGVEVHNAKTKPREEQFNSTFRVVSVLTV | |
| | VHQDWLNGKEYKCKVSNKGLPAPIEKTISKTKGQPREPQ | |
| | VYTLPPCREEMTKNQVSLWCLVKGFYPSDIAVEWESNGQ | |
| | PENNYKTTPPMLDSDGSFFLYSKLTVDKSRWQQGNVFSC | |
| | SVMHEALHNHYTQKSLSLSPG | |
| 239 | AIQLTQSPSSLSASVGDRVTITCRASQGISSALAWYQQKP | TCE010 |
| | GKAPKLLIYDASSLESGVPSRFSGSGSGTDFTLTISSLQPED | Light chain |
| | FATYYCQQFNSYPRTFGQGTKVEIKRTVAAPSVFIFPPSDE | Chain 3 amino acid |
| | QLKSGTASVVCLLNNFYPREAKVQWKVDNALQSGNSQE | sequence |
| | SVTEQDSKDSTYSLSSTLTLSKADYEKHKVYACEVTHQG | |
| | LSSPVTKSFNRGEC | |
| 240 | ZDGNEEMGGITQTP | Immunizing |
| | | peptide |
| 241 | EVQLVESGGGLVQPGNSLRLSCAASGFTFSSFGMSWVRQ | tAb0032 (synthetic |
| | APGKGLEWVSSISGSGSDTLYADSVKGRFTISRDNAKTTL | polypeptide; |
| | YLQMNSLRPEDTAVYYCTIGGSLSRSSQGTLVTVSSGGG | disclosed in WO |
| | GS | 2019/237081 A1) |
| | GGGSQVQLVQSGAEVKKPGASVKVSCKASGYTFTDYIM | |
| | HWVRQAPGQGLEWMGYINPYNDGSKYTEKFQGRVTMT | |
| | SDTSISTAYMELSRLRSDDTAVYYCARGTYYYGPQLFDY | |
| | WGQGTTVTVSSGGGGSGGGGSGGGGSDIVMTQTPLSLSV | |
| | TPGQPASISCKSSQSLETSTGTTYLNWYLQKPGQSPQLLIY | |
| | RVSKRFSGVPDRFSGSGSGTDFTLKISRVEAEDVGVYYCL | |
| | QLLEDPYTFGQGTKLEIKGGGGSGGGSDIVMTQSPDSLA | |
| | VSLGERATINCKSSQSLLNARTGKNYLAWYQQKPGQPPK | |
| | LLIYWASTRESGVPDRFSGSGSGTDFTLTISSLQAEDVAV | |
| | YYCKQSYSRRTFGGGTKVEIKGGGGSGGGGSGGGGSGG | |
| | GGSQVQLVQSGAEVKKPGASVKVSCKASGFNIKDYYMH | |
| | WVRQAPGQRLEWMGWIDLENANTIYDAKFQGRVTITRD | |
| | TSASTAYMELSSLRSEDTAVYYCARDAYGRYFYDVWGQ | |
| | GTLVTVSSHHHHHHHHHH | |
| 242 | QVQLVQSGAEVKKPGASVKVSCKASGYTFTDYIMHWVR | Anti-CD19 scFv |
| | QAPGQGLEWMGYINPYNDGSKYTEKFQGRVTMTSDTSIS | of tAb0032; |
| | TAYMELSRLRSDDTAVYYCARGTYYYGPQLFDYWGQGT | disclosed in WO |
| | TVTVSSGGGGSGGGGSGGGGSDIVMTQTPLSLSVTPGQP | 2019/237081 A1 |
| | ASISCKSSQSLETSTGTTYLNWYLQKPGQSPQLLIYRVSK | |
| | RFSGVPDRFSGSGSGTDFTLKISRVEAEDVGVYYCLQLLE | |
| | DPYTFGQGTKLEIK | |
| 243 | DIVMTQSPDSLAVSLGERATINCKSSQSLLNARTGKNYLA | Anti-CD3 scFv of |
| | WYQQKPGQPPKLLIYWASTRESGVPDRFSGSGSGTDFTL | tAb0032; |
| | TISSLQAEDVAVYYCKQSYSRRTFGGGTKVEIKGGGGSG | disclosed in WO |
| | GGGSGGGGSGGGGSQVQLVQSGAEVKKPGASVKVSCKA | 2019/237081 A1 |
| | SGFNIKDYYMHWVRQAPGQRLEWMGWIDLENANTIYD | |
| | AKFQGRVTITRDTSASTAYMELSSLRSEDTAVYYCARDA | |
| | YGRYFYDVWGQGTLVTVSS | |
| 244 | EVQLVESGGGLVQPGNSLRLSCAASGFTFSSFGMSWVRQ | Anti-HSA sdAb of |
| | APGKGLEWVSSISGSGSDTLYADSVKGRFTISRDNAKTTL | tAb0032; |
| | YLQMNSLRPEDTAVYYCTIGGSLSRSSQGTLVTVSS | disclosed in WO |
| | | 2019/237081 A1 |
| 245 | QVQLQQWGAGLLKPSETLSLTCAVYGGSFSDYYWSWVR | 13-CD19 scFv |
| | QPPGKCLEWIGEITHSGSTNYNPSLKSRVTISVDTSKNQFS | |
| | LKLSSVTAADTAVYYCARGGLDIFTGPYYFDSWGQGTLV | |
| | TVSSGGGGSGGGGSGGGGSDIQMTQSPSSLSASVGDRVTI | |
| | TCRASQEIRKDLAWYQQKPGKAPKLLIYGASSLRSGVPSR | |
| | FSGSGSGTDFTLTISSLQPEDFATYYCQQDYNYPPTFGCG | |
| | TKVEIK | |
| 246 | QVQLVESGGGVVQPGGSLRLSCAASGFTFSRYGIHWVRQ | 5-CD3 scFv |
| | APGKCLEWVAAIWYDASKTYYADSVKGRFTISRDNSKN | |
| | TLYLQMNSLRAEDTAVYYCARSNYDILTRYYQYGLDVW | |
| | GQGTTVTVSSGGGGSGGGGSGGGGSAIQLTQSPSSLSASV | |
| | GDRVTITCRASQGISSALAWYQQKPGKAPKLLIYDASSLE | |
| | SGVPSRFSGSGSGTDFTLTISSLQPEDFATYYCQQFKSYPR | |
| | TFGCGTKVEIK | |
| 247 | QVQLVESGGGVVQPGGSLRLSCAASGFTFSRYGIHWVRQ | 8-CD3 scFv |
| | APGKCLEWVAAIWYDASKTYYADSVKGRFTISRDNSKN | |
| | TLYLQMNSLRAEDTAVYYCARSNYDILTRYYSHGINVW | |
| | GQGTTVTVSSGGGGSGGGGSGGGGSAIQLTQSPSSLSASV | |
| | GDRVTITCRASQGISSALAWYQQKPGKAPKLLIYDASSLE | |
| | SGVPSRFSGSGSGTDFTLTISSLQPEDFATYYCQQFNSYPR | |
| | TFGCGTKVEIK | |
| 248 | EVQLVQSGAEVKKPGESLKISCKGSGYSFTSYWIGWVRQ | 14-CD19 scFv |
| | MPGKCLEWMGIIYPGDSDTRYSPSFQGQVTISADKSISTA | |
| | YLQWSSLKASDTAMYYCARGGTTIFGVVFEYWGQGTLV | |
| | TVSSGGGGSGGGGSGGGGSDIQMTQSPSSLSASVGDRVTI | |
| | TCRASQSINNYLNWYQQKPGKAPKLLIYGASSLQSGVPS | |
| | RFSGSGSGTDFTLTISSLQPEDFATYYCQQTYSTPWTFGC | |
| | GTKVEIK | |
| 249 | QVQLQQWGAGLLKPSETLSLTCAVYGGSFSDYYWSWVR | 1-CD19 scFv |
| | QPPGKGLEWIGEITHSGSTNYNPSLKSRVTISVDTSKNQFS | |
| | LKLSSVTAADTAVYYCARGGLDIFTGPYYFDSWGQGAL | |
| | VTVSSGGGGSGGGGSGGGGSEIVLTQSPSSLSASVGDRVT | |
| | ITCRASQEIRKDLAWYQQKPGKAPKVLIYGASSLRSGVPS | |
| | SFSGSGSGTDFTLTISSLQPEDFATYYCQQDYNYPPTFGQ | |
| | GTKVEIK | |
| 250 | QVQLVESGGGVVQPGRSLRLSCAASGFTFSRYGIHWVRQ | 1-CD3 scFv |
| | APGKGLEWVAAIWYDGSKKYYADSVKGRFTISRDNSKN | |
| | TLDLQMNSLRAEDTAVYYCARSNYDILTRYYQYGINVW | |
| | GQGTTVTVSSGGGGSGGGGSGGGGSAIQLTQSPSSLSASV | |
| | GDRVTITCRASQGISSALAWYQQKPGKAPKLLIYDASSLE | |
| | NGVPSRFSGSGSGTDFTLTISSLQPEDFATYYCQQFKSYPR | |
| | TFGQGTKVEIK | |
| 251 | QVQLVESGGGVVQPGRSLRLSCAASGFTFSRYGIHWVRQ | 3-CD3 scFv |
| | APGKGLEWVAAIWYDASKKYYADSVKGRFTISRDNSKN | |
| | TLYLQMNSLRAEDTAVYYCARSNYDILTRYYQYGLDVW | |
| | GQGTTVTVSSGGGGSGGGGSGGGGSAIQLTQSPSSLSASV | |
| | GDRVTITCRASQGISSALAWYQQKPGKAPKLLIYDASSLE | |
| | SGVPSRFSGSGSGTDFTLTISSLQPEDFATYYCQQFKSYPR | |
| | TFGQGTKVEIK | |
| 252 | QVQLQQWGAGLLKPSETLSLTCAVYGGSFSDYYWSWVR | 12-CD19 scFv |
| | QPPGKCLEWIGEITHSGSTNYNPSLKSRVTISVDTSKNQFS | |
| | LKLSSVTAADTAVYYCARGGLDIFTGPYYFDSWGQGAL | |
| | VTVSSGGGGSGGGGSGGGGSEIVLTQSPSSLSASVGDRVT | |
| | ITCRASQEIRKDLAWYQQKPGKAPKVLIYGASSLRSGVPS | |
| | SFSGSGSGTDFTLTISSLQPEDFATYYCQQDYNYPPTFGCG | |
| | TKVEIK | |
| 253 | ASTKGPSVFPLAPSSKSTSGGTAALGCLVKDYFPEPVTVS | wildtype IgG1 |
| | WNSGALTSGVHTFPAVLQSSGLYSLSSVVTVPSSSLGTQT | constant heavy |
| | YICNVNHKPSNTKVDKKVEPKSCDKTHTCPPCPAPELLG | chain |
| | GPSVFLFPPKPKDTLMISRTPEVTCVVVDVSHEDPEVKFN | |
| | WYVDGVEVHNAKTKPREEQYNSTYRVVSVLTVLHQDW | |
| | LNGKEYKCKVSNKALPAPIEKTISKAKGQPREPQVYTLPP | |
| | SREEMTKNQVSLTCLVKGFYPSDIAVEWESNGQPENNYK | |
| | TTPPVLDSDGSFFLYSKLTVDKSRWQQGNVFSCSVMHEA | |
| | LHNHYTQKSLSLSPG | |
| 254 | QVQLQQWGAGLLKPSETLSLTCAVYGGSFSDYYWSWVR | 1-CD19 Heavy |
| | QPPGKGLEWIGEITHSGSTNYNPSLKSRVTISVDTSKNQFS | Chain |
| | LKLSSVTAADTAVYYCARGGLDIFTGPYYFDSWGQGAL | (HC constant |
| | VTVSSASTKGPSVFPLAPSSKSTSGGTAALGCLVKDYFPE | region underlined) |
| | PVTVSWNSGALTSGVHTFPAVLQSSGLYSLSSVVTVPSSS | |
| | LGTQTYICNVNHKPSNTKVDKKVEPKSCDKTHTCPPCPA | |
| | PELLGGPSVFLFPPKPKDTLMISRTPEVTCVVVDVSHEDPE | |
| | VKFNWYVDGVEVHNAKTKPREEQYNSTYRVVSVLTVLH | |
| | QDWLNGKEYKCKVSNKALPAPIEKTISKAKGQPREPQVY | |
| | TLPPSREEMTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPG | |
| 255 | EIVLTQSPSSLSASVGDRVTITCRASQEIRKDLAWYQQKP | 1-CD19 Light |
| | GKAPKVLIYGASSLRSGVPSSFSGSGSGTDFTLTISSLQPE | Chain |
| | DFATYYCQQDYNYPPTFGQGTKVEIKRTVAAPSVFIFPPS | (LC constant |
| | DEQLKSGTASVVCLLNNFYPREAKVQWKVDNALQSGNS | region underlined) |
| | QESVTEQDSKDSTYSLSSTLTLSKADYEKHKVYACEVTH | |
| | QGLSSPVTKSFNRGEC | |
| 256 | QVQLVQSGAEVKKPGESLKISCKGSGYSFTSYWIGWVRQ | 2-CD19 Heavy |
| | MPGKGLELMGIIYPGDSDTRYSPSFQGQVTISADKSISTAY | Chain |
| | LQWSSLKASDTAMYYCARGGTTIFGVVFEYWGQGTLVT | (HC constant |
| | VSSASTKGPSVFPLAPSSKSTSGGTAALGCLVKDYFPEPV | region underlined) |
| | TVSWNSGALTSGVHTFPAVLQSSGLYSLSSVVTVPSSSLG | |
| | TQTYICNVNHKPSNTKVDKKVEPKSCDKTHTCPPCPAPEL | |
| | LGGPSVFLFPPKPKDTLMISRTPEVTCVVVDVSHEDPEVK | |
| | FNWYVDGVEVHNAKTKPREEQYNSTYRVVSVLTVLHQD | |
| | WLNGKEYKCKVSNKALPAPIEKTISKAKGQPREPQVYTL | |
| | PPSREEMTKNQVSLTCLVKGFYPSDIAVEWESNGQPENN | |
| | YKTTPPVLDSDGSFFLYSKLTVDKSRWQQGNVFSCSVMH | |
| | EALHNHYTQKSLSLSPG | |
| 257 | EIVLTQSPSSLSTSVGDRVTITCRASQSINNYLNWYQQKP | 2-CD19 Light |
| | GKAPKLLIYGASSLQSGVPSRFSGSGSGTDFTLTISSLQPE | Chain |
| | DFATYYCQQTYSTPWTFGQGTKVEIKRTVAAPSVFIFPPS | (LC constant |
| | DEQLKSGTASVVCLLNNFYPREAKVQWKVDNALQSGNS | region underlined) |
| | QESVTEQDSKDSTYSLSSTLTLSKADYEKHKVYACEVTH | |
| | QGLSSPVTKSFNRGEC | |
| 258 | EVQLVQSGAEVKKPGESLKISCKGSGYSFTSYWIGWVRQ | 3-CD19 Heavy |
| | MPGKGLEWMGIIYPGDSDTRYSPSFQGQVTISADKSLSTA | Chain |
| | YLQWSGLKASDTAMYYCARQSIMGVIYVAFDVWGQGT | (HC constant |
| | MVTVSSASTKGPSVFPLAPSSKSTSGGTAALGCLVKDYFP | region underlined) |
| | EPVTVSWNSGALTSGVHTFPAVLQSSGLYSLSSVVTVPSS | |
| | SLGTQTYICNVNHKPSNTKVDKKVEPKSCDKTHTCPPCP | |
| | APELLGGPSVFLFPPKPKDTLMISRTPEVTCVVVDVSHED | |
| | PEVKFNWYVDGVEVHNAKTKPREEQYNSTYRVVSVLTV | |
| | LHQDWLNGKEYKCKVSNKALPAPIEKTISKAKGQPREPQ | |
| | VYTLPPSREEMTKNQVSLTCLVKGFYPSDIAVEWESNGQ | |
| | PENNYKTTPPVLDSDGSFFLYSKLTVDKSRWQQGNVFSC | |
| | SVMHEALHNHYTQKSLSLSPG | |
| 259 | EIVLTQSPSSLSASVGDRVTITCRASQSISSYLNWYQQKPG | 3-CD19 Light |
| | KAPKLLIYAASSLQSGVPSRFSGSGSGTDFTLTITSLRPEDF | Chain |
| | ATYYCQQSYSNPWTFGQGTKVEIKRTVAAPSVFIFPPSDE | (LC constant |
| | QLKSGTASVVCLLNNFYPREAKVQWKVDNALQSGNSQE | region underlined) |
| | SVTEQDSKDSTYSLSSTLTLSKADYEKHKVYACEVTHQG | |
| | LSSPVTKSFNRGEC | |
| 260 | EVQLVQSGAEVKKPGESLKISCKGSGYSFTSYWIGWVRQ | 4-CD19 Heavy |
| | MPGKGLEWMGIIYPGDSDTRYSPSFQGQVTISADKSISTA | Chain |
| | YLQWSSLKASDTAMYYCARGGATIFGVVFDYWGQGTLV | (HC constant |
| | TVSSASTKGPSVFPLAPSSKSTSGGTAALGCLVKDYFPEP | region underlined) |
| | VTVSWNSGALTSGVHTFPAVLQSSGLYSLSSVVTVPSSSL | |
| | GTQTYICNVNHKPSNTKVDKKVEPKSCDKTHTCPPCPAP | |
| | ELLGGPSVFLFPPKPKDTLMISRTPEVTCVVVDVSHEDPE | |
| | VKFNWYVDGVEVHNAKTKPREEQYNSTYRVVSVLTVLH | |
| | QDWLNGKEYKCKVSNKALPAPIEKTISKAKGQPREPQVY | |
| | TLPPSREEMTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPG | |
| 261 | EIVLTQSPSSLSASVGDRVTITCRASQSINSYLSWFQQKPG | 4-CD19 Light |
| | KAPKLLIYAASSLQSGVPSRFSGSGSGTDFTLTISSLQPEDF | Chain |
| | ATYYCQQSYSTPPTFGQGTKVEIKRTVAAPSVFIFPPSDEQ | (LC constant |
| | LKSGTASVVCLLNNFYPREAKVQWKVDNALQSGNSQES | region underlined) |
| | VTEQDSKDSTYSLSSTLTLSKADYEKHKVYACEVTHQGL | |
| | SSPVTKSFNRGEC | |
| 262 | QVQLQESGPGLVKPSETLSLTCTVSGGSISSYYWSWIRQP | 5-CD19 Heavy |
| | PGKGLEWIGYIYYSGSTKYNPSLKSRVTISVDTSKNQFSL | Chain |
| | QLSSVTAADTAVYYCATGLAAAGDYYYGMDVWGQGTT | (HC constant |
| | VTVSSASTKGPSVFPLAPSSKSTSGGTAALGCLVKDYFPE | region underlined) |
| | PVTVSWNSGALTSGVHTFPAVLQSSGLYSLSSVVTVPSSS | |
| | LGTQTYICNVNHKPSNTKVDKKVEPKSCDKTHTCPPCPA | |
| | PELLGGPSVFLFPPKPKDTLMISRTPEVTCVVVDVSHEDPE | |
| | VKFNWYVDGVEVHNAKTKPREEQYNSTYRVVSVLTVLH | |
| | QDWLNGKEYKCKVSNKALPAPIEKTISKAKGQPREPQVY | |
| | TLPPSREEMTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPG | |
| 263 | EIVLTQSPSSLSASVGDRVTITCRASQGISSALAWYQQKPG | 5-CD19 Light |
| | KAPKLLIYDASSLESGVPSRFSGGGSGTDFTLTISSLQPEDF | Chain |
| | ASYYCQQFNSYPLTFGGGTKVEIKRTVAAPSVFIFPPSDEQ | (LC constant |
| | LKSGTASVVCLLNNFYPREAKVQWKVDNALQSGNSQES | region underlined) |
| | VTEQDSKDSTYSLSSTLTLSKADYEKHKVYACEVTHQGL | |
| | SSPVTKSFNRGEC | |
| 264 | QVQLVQSGAEVKKPGESLKISCKGSGYSFTSYWIGWVRQ | 6-CD19 Heavy |
| | MPGKGLEWMGIIYPGDSDTRYSPSFQGQVTISADKSISTA | Chain |
| | YLQWSSLKASDTAMYYCARQSIVATITWAFDIWGQGTM | (HC constant |
| | VTVSSASTKGPSVFPLAPSSKSTSGGTAALGCLVKDYFPE | region underlined) |
| | PVTVSWNSGALTSGVHTFPAVLQSSGLYSLSSVVTVPSSS | |
| | LGTQTYICNVNHKPSNTKVDKKVEPKSCDKTHTCPPCPA | |
| | PELLGGPSVFLFPPKPKDTLMISRTPEVTCVVVDVSHEDPE | |
| | VKFNWYVDGVEVHNAKTKPREEQYNSTYRVVSVLTVLH | |
| | QDWLNGKEYKCKVSNKALPAPIEKTISKAKGQPREPQVY | |
| | TLPPSREEMTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPG | |
| 265 | EIVLTQSPSFLSASVGDRVTITCRASQGISSYLAWYQQKPG | 6-CD19 Light |
| | KAPKLLIYAASTLQSGVPSRFSGSGSGTEFTLTVSSLQPED | Chain |
| | FATYYCQQLNSYPYTFGQGTKLEIKRTVAAPSVFIFPPSDE | (LC constant |
| | QLKSGTASVVCLLNNFYPREAKVQWKVDNALQSGNSQE | region underlined) |
| | SVTEQDSKDSTYSLSSTLTLSKADYEKHKVYACEVTHQG | |
| | LSSPVTKSFNRGEC | |
| 266 | QVQLQESGPGLVKPSETLSLTCTVSGGSISSGSYNWGWIR | 7-CD19 Heavy |
| | QPPGKGLEWIGSIYYSGSTYYNPSLKSRVTISGDTSKNQFS | Chain |
| | LKLSSLTAADTAVYYCARHTGGFDYWGQGTLVTVSSAS | (HC constant |
| | TKGPSVFPLAPSSKSTSGGTAALGCLVKDYFPEPVTVSWN | region underlined) |
| | SGALTSGVHTFPAVLQSSGLYSLSSVVTVPSSSLGTQTYIC | |
| | NVNHKPSNTKVDKKVEPKSCDKTHTCPPCPAPELLGGPS | |
| | VFLFPPKPKDTLMISRTPEVTCVVVDVSHEDPEVKENWY | |
| | VDGVEVHNAKTKPREEQYNSTYRVVSVLTVLHQDWLNG | |
| | KEYKCKVSNKALPAPIEKTISKAKGQPREPQVYTLPPSRE | |
| | EMTKNQVSLTCLVKGFYPSDIAVEWESNGQPENNYKTTP | |
| | PVLDSDGSFFLYSKLTVDKSRWQQGNVFSCSVMHEALH | |
| | NHYTQKSLSLSPG | |
| 267 | EIVLTQSPATLSVSPGERATLSCRASQSVSSNLAWYQQKP | |
| | GQAPRLLIYGASIRATGIPARFSGSGSGTEFTLTISSLQSED | 7-CD19 Light |
| | FAVYYCQQYNNWPLTFGGGTKVEIKRTVAAPSVFIFPPSD | Chain |
| | EQLKSGTASVVCLLNNFYPREAKVQWKVDNALQSGNSQ | (LC constant |
| | ESVTEQDSKDSTYSLSSTLTLSKADYEKHKVYACEVTHQ | region underlined) |
| | GLSSPVTKSFNRGEC | |
| 268 | QVQLVQSGAEVKKPGESLRISCKGSGYSFSSYWIAWVRQ | 8-CD19 Heavy |
| | MPGKGLEWMGIIYPGDSDTRYSPSFQGQVTISADKSISTA | Chain |
| | YLQWSSLKASDTAMYYCARHGGYNYGFTDYYYGMDV | (HC constant |
| | WGQGTTVTVSSASTKGPSVFPLAPSSKSTSGGTAALGCLV | region underlined) |
| | KDYFPEPVTVSWNSGALTSGVHTFPAVLQSSGLYSLSSVV | |
| | TVPSSSLGTQTYICNVNHKPSNTKVDKKVEPKSCDKTHT | |
| | CPPCPAPELLGGPSVFLFPPKPKDTLMISRTPEVTCVVVDV | |
| | SHEDPEVKFNWYVDGVEVHNAKTKPREEQYNSTYRVVS | |
| | VLTVLHQDWLNGKEYKCKVSNKALPAPIEKTISKAKGQP | |
| | REPQVYTLPPSREEMTKNQVSLTCLVKGFYPSDIAVEWES | |
| | NGQPENNYKTTPPVLDSDGSFFLYSKLTVDKSRWQQGNV | |
| | FSCSVMHEALHNHYTQKSLSLSPG | |
| 269 | DIVMTQTPLSSPVTLGQPASISCRSSQRLVHSDGDTYLSW | 8-CD19 Light |
| | LQQRPGQPPRLLIYKISNRFSGVPDRFSGSGAGTDFTLKIS | Chain |
| | RVEAEDVGVYYCMQATQFPYTFGQGTKLEIKRTVAAPSV | (LC constant |
| | FIFPPSDEQLKSGTASVVCLLNNFYPREAKVQWKVDNAL | region underlined) |
| | QSGNSQESVTEQDSKDSTYSLSSTLTLSKADYEKHKVYA | |
| | CEVTHQGLSSPVTKSFNRGEC | |
| 270 | EVQLVQSGAEVKKPGESLKISCKGSGYSFTSYWIGWVRQ | 9-CD19 Heavy |
| | MPGKGLEWMGIIYPGDSDTRYSPSFQGQVAISADKSISTA | Chain |
| | YLQWSSLKASDTAMYYCARRSIAGTTSWSFDLWGRGTL | (HC constant |
| | VTVSSASTKGPSVFPLAPSSKSTSGGTAALGCLVKDYFPE | region underlined) |
| | PVTVSWNSGALTSGVHTFPAVLQSSGLYSLSSVVTVPSSS | |
| | LGTQTYICNVNHKPSNTKVDKKVEPKSCDKTHTCPPCPA | |
| | PELLGGPSVFLFPPKPKDTLMISRTPEVTCVVVDVSHEDPE | |
| | VKFNWYVDGVEVHNAKTKPREEQYNSTYRVVSVLTVLH | |
| | QDWLNGKEYKCKVSNKALPAPIEKTISKAKGQPREPQVY | |
| | TLPPSREEMTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPG | |
| 271 | EIVLTQSPSSLSASVGDRVTITCRTSQDIRNDLGWYQQKP | 9-CD19 Light |
| | GNAPKLLIYAASSLQSGVPSRFSGSGSGTDFTLTISSLQPE | Chain |
| | DFATYYCLQDYNYPLTFGGGTKVEIKRTVAAPSVFIFPPS | (LC constant |
| | DEQLKSGTASVVCLLNNFYPREAKVQWKVDNALQSGNS | region underlined) |
| | QESVTEQDSKDSTYSLSSTLTLSKADYEKHKVYACEVTH | |
| | QGLSSPVTKSFNRGEC | |
| 272 | QVQLQESGPGLVKPSETLSLTCTVSGGSFSSYYWSWIRQP | 10-CD19 Heavy |
| | PGKGLEWIGYIYYSGRTNYNPSLKSRVTISIDTSKNQVSLK | Chain |
| | LSSVTAADTAVYYCASHHWGSALDYWGQGTLVTVSSAS | (HC constant |
| | TKGPSVFPLAPSSKSTSGGTAALGCLVKDYFPEPVTVSWN | region underlined) |
| | SGALTSGVHTFPAVLQSSGLYSLSSVVTVPSSSLGTQTYIC | |
| | NVNHKPSNTKVDKKVEPKSCDKTHTCPPCPAPELLGGPS | |
| | VFLFPPKPKDTLMISRTPEVTCVVVDVSHEDPEVKENWY | |
| | VDGVEVHNAKTKPREEQYNSTYRVVSVLTVLHQDWLNG | |
| | KEYKCKVSNKALPAPIEKTISKAKGQPREPQVYTLPPSRE | |
| | EMTKNQVSLTCLVKGFYPSDIAVEWESNGQPENNYKTTP | |
| | PVLDSDGSFFLYSKLTVDKSRWQQGNVFSCSVMHEALH | |
| | NHYTQKSLSLSPG | |
| 273 | EIVLTQSPLSLPVTLGQPASISCRSSQSLVYSDGNTYLNWF | 10-CD19 Light |
| | QQRPGQSPRRLIYKVSNRDSGVPDRFSGSGSGTDFTLKISR | Chain |
| | VEAEDVGVYYCMQGTHWPWTFGQGTKVEIKRTVAAPS | (LC constant |
| | VFIFPPSDEQLKSGTASVVCLLNNFYPREAKVQWKVDNA | region underlined) |
| | LQSGNSQESVTEQDSKDSTYSLSSTLTLSKADYEKHKVY | |
| | ACEVTHQGLSSPVTKSFNRGEC | |
| 274 | QVQLQQWGAGLLKPSETLSLTCAVYGGSFSDYYWSWVR | 11-CD19 Heavy |
| | QPPGKGLEWIGEITHSGSTNYNPSLKSRVTISVDTSKNQFS | Chain |
| | LKLSSVTAADTAVYYCARGGLDIFTGPYYFDSWGQGTLV | (HC constant |
| | TVSSASTKGPSVFPLAPSSKSTSGGTAALGCLVKDYFPEP | region underlined) |
| | VTVSWNSGALTSGVHTFPAVLQSSGLYSLSSVVTVPSSSL | |
| | GTQTYICNVNHKPSNTKVDKKVEPKSCDKTHTCPPCPAP | |
| | ELLGGPSVFLFPPKPKDTLMISRTPEVTCVVVDVSHEDPE | |
| | VKFNWYVDGVEVHNAKTKPREEQYNSTYRVVSVLTVLH | |
| | QDWLNGKEYKCKVSNKALPAPIEKTISKAKGQPREPQVY | |
| | TLPPSREEMTKNQVSLTCLVKGFYPSDIAVEWESNGQPEN | |
| | NYKTTPPVLDSDGSFFLYSKLTVDKSRWQQGNVFSCSVM | |
| | HEALHNHYTQKSLSLSPG | |
| 275 | DIQMTQSPSSLSASVGDRVTITCRASQEIRKDLAWYQQKP | 11-CD19 Light |
| | GKAPKLLIYGASSLRSGVPSRFSGSGSGTDFTLTISSLQPE | Chain |
| | DFATYYCQQDYNYPPTFGQGTKVEIKRTVAAPSVFIFPPS | (LC constant |
| | DEQLKSGTASVVCLLNNFYPREAKVQWKVDNALQSGNS | region underlined) |
| | QESVTEQDSKDSTYSLSSTLTLSKADYEKHKVYACEVTH | |
| | QGLSSPVTKSFNRGEC | |
| 276 | AIWYDX₁SX₂X₃YYADSVKG, X₁ = G or A or S, X₂ = K or H or | CDR-H2 |
| | N, X₃ = K or T or D | consensus |
| 277 | QQFX₂SYPRT, X₂ = R or N or K | CDR-L3 |
| | | consensus |
| 278 | QVQLVESGGGVVQPGX₁SLRLSCAASGFTFSX₂X₃GIHWV | CD3 VH |
| | RQAPGKX₄LEWVAAIWYDX₅SX₆X₇YYADSVKGRFTISRD | consensus |
| | NSX₈NTLX₉LQMNSLRAEDTAVYYCARSNX₁₀DX₁₁LTX₁₂X | |
| | ₁₃YX₁₄X₁₅GX₁₆X₁₇VWGQGTTVTVSS, wherein X₁ is R, G, or | |
| | E; X₂ is R or N; X₃ is Y or H, X₄ is G or C; X₅ is G or A or S; X₆ | |
| | is K, H, or N, X₇ is K, T, or D; X₈ is K, E, or S; X₉ is Y or D; | |
| | X₁₀ is Y or F, X₁₁ is I or V, X₁₂ is R or G; X₁₃ is Y or H; X₁₄ is Q | |
| | or S; X₁₅ is Y or H; X₁₆ is I or L; and X₁₇ is N or D | |

1. A bispecific antibody comprising (a) a first binding domain that binds CD19 and (b) a second binding domain that binds CD3, wherein:
the first binding domain that binds CD19 comprises a heavy chain variable (VH) region and a light chain variable (VL) region, wherein:
(a) the VH region comprises a heavy chain complementarity determining region 1 (CDR-H1) comprising the amino acid sequence set forth in any one of SEQ ID NOs: 9, 118, 131, 140, or 146, a heavy chain complementarity determining region 2 (CDR-H2) comprising the amino acid sequence set forth in any one of SEQ ID NOs: 10, 119, 132, 141, or 154, and a heavy chain complementarity determining region 3 (CDR-H3) comprising the amino acid sequence set forth in any one of SEQ ID NOs: 11, 120, 124, 128, 133, 136, 142, 147, 151, or 155; and
the VL region comprises a light chain complementarity determining region 1 (CDR-L1) comprising the amino acid sequence set forth in SEQ ID NOs: 14, 121, 125, 129, 134, 137, 143, 148, 152, or 156; a light chain complementarity determining region 2 (CDR-L2) comprising the amino acid sequence set forth in any one of SEQ ID NOs: 15, 122, 126, 25, 138, 144, 149, or 157, and a light chain complementarity determining region 3 (CDR-L3) comprising the amino acid sequence set forth in any one of SEQ ID NOs: 16, 123, 127, 130, 135, 139, 145, 150, 153, or 158; or
(b) the VH region comprises a CDR-H1, a CDR-H2, and a CDR-H3 contained within any one of SEQ ID NOs: 12, 32, 34, 44, 46, 48, 50, 52, 54, 56, 58, 60, 62, or 88, and the VL region comprises a CDR-L1, a CDR-L2, and a CDR-L3 contained within any one of SEQ ID NOs: 17, 33, 35, 45, 47, 49, 51, 53, 55, 57, 59, 61, 63, or 89.

2. The bispecific antibody of claim 1, wherein in the first binding domain:
the CDR-H1 comprises the amino acid sequence set forth in any one of SEQ ID NOs: 9, 118, 131, 140, or 146, the CDR-H2 comprises the amino acid sequence set forth in any one of SEQ ID NOs: 10, 119, 132, 141, or 154, and the CDR-H3 comprises the amino acid sequence set forth in any one of SEQ ID NOs: 11, 120, 124, 128, 133, 136, 142, 147, 151, or 155; and
the CDR-L1 comprises the amino acid sequence set forth in SEQ ID NOs: 14, 121, 125, 129, 134, 137, 143, 148, 152, or 156, the CDR-L2 comprises the amino acid sequence set forth in any one of SEQ ID NOs: 15, 122, 126, 25, 138, 144, 149, or 157, and the CDR-L3 comprises the amino acid sequence set forth in any one of SEQ ID NOs: 16, 123, 127, 130, 135, 139, 145, 150, 153, or 158.

3. The bispecific antibody of claim 1 or claim 2, wherein in the first binding domain:
the VH region comprises the CDR-H1, the CDR-H2, and the CDR-H3 contained within SEQ ID NO: 32, and the VL region comprises the CDR-L1, the CDR-L2, and the CDR-L3 contained within SEQ ID NO: 33;
the VH region comprises the CDR-H1, the CDR-H2, and the CDR-H3 contained within SEQ ID NO: 34, and the VL region comprises the CDR-L1, the CDR-L2, and the CDR-L3 contained within SEQ ID NO: 35;
the VH region comprises the CDR-H1, the CDR-H2, and the CDR-H3 contained within SEQ ID NO: 44, and the VL region comprises the CDR-L1, the CDR-L2, and the CDR-L3 contained within SEQ ID NO: 45;
the VH region comprises the CDR-H1, the CDR-H2, and the CDR-H3 contained within SEQ ID NO: 46, and the VL region comprises the CDR-L1, the CDR-L2, and the CDR-L3 contained within SEQ ID NO: 47;
the VH region comprises the CDR-H1, the CDR-H2, and the CDR-H3 contained within SEQ ID NO: 48, and the VL region comprises the CDR-L1, the CDR-L2, and the CDR-L3 contained within SEQ ID NO: 49;
the VH region comprises the CDR-H1, the CDR-H2, and the CDR-H3 contained within SEQ ID NO: 50, and the VL region comprises the CDR-L1, the CDR-L2, and the CDR-L3 contained within SEQ ID NO: 51;
the VH region comprises the CDR-H1, the CDR-H2, and the CDR-H3 contained within SEQ ID NO: 52, and the VL region comprises the CDR-L1, the CDR-L2, and the CDR-L3 contained within SEQ ID NO: 53;
the VH region comprises the CDR-H1, the CDR-H2, and the CDR-H3 contained within SEQ ID NO: 54, and the VL region comprises the CDR-L1, the CDR-L2, and the CDR-L3 contained within SEQ ID NO: 55;
the VH region comprises the CDR-H1, the CDR-H2, and the CDR-H3 contained within SEQ ID NO: 56, and the VL region comprises the CDR-L1, the CDR-L2, and the CDR-L3 contained within SEQ ID NO: 57;
the VH region comprises the CDR-H1, the CDR-H2, and the CDR-H3 contained within SEQ ID NO: 58, and the VL region comprises the CDR-L1, the CDR-L2, and the CDR-L3 contained within SEQ ID NO: 59;
the VH region comprises the CDR-H1, the CDR-H2, and the CDR-H3 contained within SEQ ID NO: 60, and the VL region comprises the CDR-L1, the CDR-L2, and the CDR-L3 contained within SEQ ID NO: 61;
the VH region comprises the CDR-H1, the CDR-H2, and the CDR-H3 contained within SEQ ID NO: 62, and the VL region comprises the CDR-L1, the CDR-L2, and the CDR-L3 contained within SEQ ID NO: 63;
the VH region comprises the CDR-H1, the CDR-H2, and the CDR-H3 contained within SEQ ID NO: 12, and the VL region comprises the CDR-L1, the CDR-L2, and the CDR-L3 contained within SEQ ID NO: 17; or
the VH region comprises the CDR-H1, the CDR-H2, and the CDR-H3 contained within SEQ ID NO: 88, and the VL region comprises the CDR-L1, the CDR-L2, and the CDR-L3 contained within SEQ ID NO: 89.

4. The bispecific antibody of any one of claims 1-3, wherein in the first binding domain:
the VH region comprises the CDR-H1, the CDR-H2, and the CDR-H3 comprising the amino acid sequence set forth in SEQ ID NOS: 9, 10, and 11, respectively, and the VL region comprises the CDR-L1, the CDR-L2, and the CDR-L3 comprising the amino acid sequence set forth in SEQ ID NOS: 14, 15, and 16, respectively;
the VH region comprises the CDR-H1, the CDR-H2, and the CDR-H3 comprising the amino acid sequence set forth in SEQ ID NOS: 118, 119, and 120, respectively, and the VL region comprises the CDR-L1, the CDR-L2, and the CDR-L3 comprising the amino acid sequence set forth in SEQ ID NOS: 121, 122, and 123, respectively;
the VH region comprises the CDR-H1, the CDR-H2, and the CDR-H3 comprising the amino acid sequence set forth in SEQ ID NOS:118, 119, and 124, respectively, and the VL region comprises the CDR-L1, the CDR-L2, and the CDR-L3 comprising the amino acid sequence set forth in SEQ ID NOS:125, 126, and 127, respectively;
the VH region comprises the CDR-H1, the CDR-H2, and the CDR-H3 comprising the amino acid sequence set forth in SEQ ID NOS:118, 119, and 128, respectively, and the VL region comprises the CDR-L1, the CDR-L2, and the CDR-L3 comprising the amino acid sequence set forth in SEQ ID NOS: 129, 126, and 130, respectively;
the VH region comprises the CDR-H1, the CDR-H2, and the CDR-H3 comprising the amino acid sequence set forth in SEQ ID NOS: 131, 132, and 133, respectively, and the VL region comprises the CDR-L1, the CDR-L2, and the CDR-L3 comprising the amino acid sequence set forth in SEQ ID NOS: 134, 25, and 135, respectively;
the VH region comprises the CDR-H1, the CDR-H2, and the CDR-H3 comprising the amino acid sequence set forth in SEQ ID NOS: 118, 119, and 136, respectively, and the VL region comprises the CDR-L1, the CDR-L2, and the CDR-L3 comprising the amino acid sequence set forth in SEQ ID NOS:137, 138, and 139, respectively;
the VH region comprises the CDR-H1, the CDR-H2, and the CDR-H3 comprising the amino acid sequence set forth in SEQ ID NOS:140, 141, and 142, respectively, and the VL region comprises the CDR-L1, the CDR-L2, and the CDR-L3 comprising the amino acid sequence set forth in SEQ ID NOS: 143, 144, and 145, respectively;
the VH region comprises the CDR-H1, the CDR-H2, and the CDR-H3 comprising the amino acid sequence set forth in SEQ ID NOS: 146, 119, and 147, respectively, and the VL region comprises the CDR-L1, the CDR-L2, and the CDR-L3 comprising the amino acid sequence set forth in SEQ ID NOS: 148, 149, and 150, respectively;
the VH region comprises the CDR-H1, the CDR-H2, and the CDR-H3 comprising the amino acid sequence set forth in SEQ ID NOS: 118, 119, and 151, respectively, and the VL region comprises the CDR-L1, the CDR-L2, and the CDR-L3 comprising the amino acid sequence set forth in SEQ ID NOS: 152, 126, and 153, respectively; or
the VH region comprises the CDR-H1, the CDR-H2, and the CDR-H3 comprising the amino acid sequence set forth in SEQ ID NOS:131, 154, and 155, respectively, and the VL region comprises the CDR-L1, the CDR-L2, and the CDR-L3 comprising the amino acid sequence set forth in SEQ ID NOS: 156, 157, and 158, respectively.

5. The bispecific antibody of any one of claims 1-4, wherein in the first binding domain:
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 44, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 45;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 46, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 47;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 48, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 49;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 50, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 51;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 52, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 53;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 54, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 55;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 56, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 57;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 58, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 59;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 60, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 61;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 62, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 63;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 12, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 17; or
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 88, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 89.

6. The bispecific antibody of any one of claims 1-5, wherein in the first binding domain:
the VH region and the VL region comprise the amino acid sequences set forth in SEQ ID NO: 32 and 33, respectively;
the VH region and the VL region comprise the amino acid sequences set forth in SEQ ID NO: 34 and 35, respectively;
the VH region and the VL region comprise the amino acid sequences set forth in SEQ ID NO: 44 and 45, respectively;
the VH region and the VL region comprise the amino acid sequences set forth in SEQ ID NO: 46 and 47, respectively;
the VH region and the VL region comprise the amino acid sequences set forth in SEQ ID NO: 48 and 49, respectively;
the VH region and the VL region comprise the amino acid sequences set forth in SEQ ID NO: 50 and 51, respectively;
the VH region and the VL region comprise the amino acid sequences set forth in SEQ ID NO: 52 and 53, respectively;
the VH region and the VL region comprise the amino acid sequences set forth in SEQ ID NO: 54 and 55, respectively;
the VH region and the VL region comprise the amino acid sequences set forth in SEQ ID NO: 56 and 57, respectively;
the VH region and the VL region comprise the amino acid sequences set forth in SEQ ID NO: 58 and 59, respectively;
the VH region and the VL region comprise the amino acid sequences set forth in SEQ ID NO: 60 and 61, respectively;
the VH region and the VL region comprise the amino acid sequences set forth in SEQ ID NO: 62 and 63, respectively;
the VH region and the VL region comprise the amino acid sequences set forth in SEQ ID NO: 12 and 17, respectively; or
the VH region and the VL region comprise the amino acid sequences set forth in SEQ ID NO: 88 and 89, respectively.

7. The bispecific antibody of any one of claims 1-6, wherein the first binding domain is an scFv.

8. The bispecific antibody of claim 7, wherein the scFv comprises a VH-linker-VL arrangement.

9. The bispecific antibody of claim 7, wherein the scFv comprises a VL-linker-VH arrangement.

10. The bispecific antibody of any claim 8 or 9, wherein the linker is a peptide linker comprising (Gly4Ser)3 (SEQ ID NO: 162).

11. The bispecific antibody of any of claims 1-10, wherein the first binding domain is an scFv that is a disulfide-stabilized scFv.

12. The bispecific antibody of any one of claims 1-11, wherein the first binding domain comprises:
(a) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 245;
(b) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 248;
(c) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 249; or
(d) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 252.

13. The bispecific antibody of any one of claims 1-12, wherein the first binding domain comprises:
(a) the amino acid sequence of SEQ ID NO: 245;
(b) the amino acid sequence of SEQ ID NO: 248;
(c) the amino acid sequence of SEQ ID NO: 249; or
(d) the amino acid sequence of SEQ ID NO: 252.

14. The bispecific antibody of any one of claims 1-6, wherein, in the first binding domain, the VH region comprises the CDR-H1, a CDR-H2, and a CDR-H3 comprising the amino acid sequence set forth in SEQ ID NOs: 9, 10, and 11, respectively, and the VL region comprises a CDR-L1, a CDR-L2, and a CDR-L3 comprising the amino acid sequence set forth in SEQ ID NOS: 14, 15, and 16, respectively.

15. The bispecific antibody of any one of claims 1-6 and 14, wherein, in the first binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 12, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 17.

16. The bispecific antibody of any one of claims 1-6, 14 and 15, wherein the VH region and the VL region comprises the amino acid sequences set forth in SEQ ID NOs: 12 and 17, respectively.

17. The bispecific antibody of any one of claims 1-6, wherein, in the first binding domain, the VH region comprises the CDR-H1, a CDR-H2, and a CDR-H3 comprising the sequence set forth in SEQ ID NOs: 118, 119, and 120, respectively, and the VL region comprises a CDR-L1, a CDR-L2, and a CDR-L3 comprising the sequence set forth in SEQ ID NOS: 121, 122, 123, respectively.

18. The bispecific antibody of any one of claims 1-6 and 17, wherein, in the first binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 88, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 89.

19. The bispecific antibody of any one of claims 1-6, 17 and 18, wherein, in the first binding domain, the VH region and the VL region comprises the sequence set forth in SEQ ID NOs: 88 and 89, respectively.

20. The bispecific antibody of any one of claims 1-19, wherein the second binding domain that binds CD3 comprises a heavy chain variable (VH) region and a light chain variable (VL) region, wherein:
the VH comprises a heavy chain complementarity determining region 1 (CDR-H1) comprising the amino acid sequence set forth in SEQ ID NO: 198, a heavy chain complementarity determining region 2 (CDR-H2) comprising the amino acid sequence set forth in SEQ ID NO: 276, and a heavy chain complementarity determining region 3 (CDR-H3) comprising the amino acid sequence set forth in SEQ ID NO: 200; and
the VL comprises a light chain complementarity determining region 1 (CDR-L1) comprising the amino acid sequence set forth in SEQ ID NO: 24, a light chain complementarity determining region 2 (CDR-L2) comprising the amino acid sequence set forth in SEQ ID NO: 201, and a light chain complementarity determining region 3 (CDR-L3) comprising the amino acid sequence set forth within SEQ ID NO: 277.

21. The bispecific antibody of any one of claims 1-20, wherein in the second binding domain, the VH comprises a heavy chain complementarity determining region 1 (CDR-H1) comprising the amino acid sequence set forth in SEQ ID NO: 198, a heavy chain complementarity determining region 2 (CDR-H2) comprising the amino acid sequence set forth in SEQ ID NO: 199, and a heavy chain complementarity determining region 3 (CDR-H3) comprising the amino acid sequence set forth in SEQ ID NO: 200; and
the VL comprises a light chain complementarity determining region 1 (CDR-L1) comprising the amino acid sequence set forth in SEQ ID NO: 24, a light chain complementarity determining region 2 (CDR-L2) comprising the amino acid sequence set forth in SEQ ID NO: 201, and a light chain complementarity determining region 3 (CDR-L3) comprising the amino acid sequence set forth within SEQ ID NO: 106.

22. The bispecific antibody of any one of claims 1-21, wherein, in the second binding domain, the VH region binding domain and the VL region comprise, respectively:
a VH region sequence of the consensus sequence set forth in QVQLVESGGGVVQPGX₁SLRLSCAASGFTFSX₂X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁L TX₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G or A or S; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 278); and
a VL region sequence of the consensus sequence set forth in AIQLTQSPSSLSASVGDRVTITCRASQGISSALAWYQQKPGKAPKLLIYDASX₁LEX₂G VPSRFSGSGSGTDFTLTISSLQPEDFATYYCQQFX₃SYPRTFGX₄GTKVEIK, wherein X₁ is S or G, X₂ is N or S; X₃ is K or N; and X₄ is Q or C (SEQ ID NO: 197).

23. The bispecific antibody of any one of claims 1-22, wherein, in the second binding domain, the VH region binding domain and the VL region comprise, respectively:
a VH region sequence of the consensus sequence set forth in QVQLVESGGGVVQPGX₁SLRLSCAASGFTFSX₂X₃GIHWVRQAPGKX₄LEWVAAIWY DX₅SX₆X₇YYADSVKGRFTISRDNSX₈NTLX₉LQMNSLRAEDTAVYYCARSNX₁₀DX₁₁L TX₁₂X₁₃YX₁₄X₁₅GX₁₆X₁₇VWGQGTTVTVSS, wherein X₁ is R, G, or E; X₂ is R or N; X₃ is Y or H, X₄ is G or C; X₅ is G or A; X₆ is K, H, or N, X₇ is K, T, or D; X₈ is K, E, or S; X₉ is Y or D; X₁₀ is Y or F, X₁₁ is I or V, X₁₂ is R or G; X₁₃ is Y or H; X₁₄ is Q or S; X₁₅ is Y or H; X₁₆ is I or L; and X₁₇ is N or D; (SEQ ID NO: 196); and
a VL region sequence of the consensus sequence set forth in AIQLTQSPSSLSASVGDRVTITCRASQGISSALAWYQQKPGKAPKLLIYDASX₁LEX₂G VPSRFSGSGSGTDFTLTISSLQPEDFATYYCQQFX₃SYPRTFGX₄GTKVEIK, wherein X₁ is S or G, X₂ is N or S; X₃ is K or N; and X₄ is Q or C (SEQ ID NO: 197).

24. The bispecific antibody of any one of claims 20-23, wherein in the second binding domain:
the CDR-H1 comprises the sequence set forth in any one of SEQ ID NOs: 19, 109, 202, or 203, the CDR-H2 comprises the sequence set forth in any one of SEQ ID NOs: 20, 107, 110, 112, or 204-208, and the CDR-H3 comprises the sequence set forth in any one of SEQ ID NOs: 21, 108, 111, 113, or 209-214; and
the CDR-L1 comprises the sequence set forth in SEQ ID NOs: 24, the CDR-L2 comprises the sequence set forth in any one of SEQ ID NOs: 25, 114, 215, or 216, and the CDR-L3 comprises the sequence set forth in any one of SEQ ID NOs: 26 or 115.

25. The bispecific antibody of any one of claims 20-24, wherein in the second binding domain:
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NO: 37;
the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 40, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 42, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 22, and the VL region comprises the CDR-L1, the CDR-L2, and the CDR-L3 contained within SEQ ID NO: 27;
the VH region comprises the CDR-H1, the CDR-H2, and the CDR-H3 contained within SEQ ID NO: 68, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 70, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 72, and the VL region comprises the CDR-L1, the CDR-L2, and the CDR-L3 contained within SEQ ID NO: 73;
the VH region comprises the CDR-H1, the CDR-H2, and the CDR-H3 contained within SEQ ID NO: 74, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 78, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 80, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 82, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 84, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 86, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 159, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 195, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 41, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 64, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 65, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 66, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 67, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 69, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 71, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 75, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 76, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 77, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 79, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 81, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 83, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 85, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 87, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 90, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 91, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 92, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 93, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 94, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 95, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 96, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 97, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 98, and the VL region comprises the CDR-L1, the CDR-L2, and the CDR-L3 contained within SEQ ID NOs: 37, 39, or 43;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 39;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 99;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 43;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 100;
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NOs: 101;
the VH region comprises the CDR-H1, the CDR-H2, and the CDR-H3 contained within SEQ ID NO: 42, and the VL region comprises the CDR-L1, the CDR-L2, and the CDR-L3 contained within SEQ ID NOs: 37;
the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NOs: 37; or
the VH region comprises the CDR-H1, the CDR-H2, and the CDR-H3 contained within SEQ ID NO: 40, and the VL region comprises the CDR-L1, the CDR-L2, and the CDR-L3 contained within SEQ ID NOs: 37.

26. The bispecific antibody of any one of claims 20-25, wherein in the second binding domain:
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 109, 110, and 108, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 110, and 21, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 20, and 21, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 20, and 111, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 112, and 21, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 20, and 113, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 204, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 205, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 206, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 209, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 113, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 210, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 211, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 111, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 109, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 202, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 203, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 212, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 213, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 214, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 21, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 207, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 208, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 110, and 113, respectively, and the VL region comprises the CDR-L1, the CDR-L2, and the CDR-L3 comprising the sequence set forth in SEQ ID NOS: 24, 114, and 26, respectively
the VH region comprises the CDR-H1, the CDR-H2, and the CDR-H3 comprising the sequence set forth in SEQ ID NOS: 19, 110, and 210, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 202, 110, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 110, and 214, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 115, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 215, and 26, respectively;
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 216, and 115, respectively;
the VH region comprises the CDR-H1, the CDR-H2, and the CDR-H3 comprising the sequence set forth in SEQ ID NOS: 19, 110, and 111, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 109, 110, 108, respectively, and the VL region comprises the CDR-L1, the CDR-L2, and the CDR-L3 comprising the sequence set forth in SEQ ID NOS: 24, 114, and 26, respectively; or
the VH region comprises the CDR-H1, the CDR-H2, and the CDR-H3 comprising the sequence set forth in SEQ ID NOS: 19, 110, and 21, respectively, and the VL region comprises the CDR-L1, the CDR-L2, and the CDR-L3 comprising the sequence set forth in SEQ ID NOS: 24, 114, and 26, respectively.

27. The bispecific antibody of any one of claims 20-26, wherein in the second binding domain:
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 22, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 68, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 70, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 72, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 73;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 74, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 78, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 80, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 82, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 84, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 86, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 159, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 195, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 41, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 64, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 65, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 66, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 67, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 69, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 71, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 75, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 76, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 77, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 79, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 81, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 83, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 85, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 87, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 90, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 91, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 92, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 93, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 94, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 95, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 96, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 97, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 98, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NOs: 37, 39, or 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 99;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 100;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 101;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37; or
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37.

28. The bispecific antibody of any one of claims 20-27, wherein in the second binding domain:
the VH region and the VL region comprise the sequences set forth in SEQ ID NO: 36 and 37, respectively;
the VH region and the VL region comprise the sequences set forth in SEQ ID NO: 38 and 39, respectively;
the VH region and the VL region comprise the sequences set forth in SEQ ID NO: 40 and 39, respectively;
the VH region and the VL region comprise the sequences set forth in SEQ ID NO: 42 and 43, respectively;
the VH region and the VL region comprise the sequences set forth in SEQ ID NO: 22 and 27, respectively;
the VH region and the VL region comprise the sequences set forth in SEQ ID NO: 68 and 39, respectively;
the VH region and the VL region comprise the sequences set forth in SEQ ID NO: 70 and 43, respectively;
the VH region and the VL region comprise the sequences set forth in SEQ ID NO: 72 and 73, respectively;
the VH region and the VL region comprise the sequences set forth in SEQ ID NO: 74 and 39, respectively;
the VH region and the VL region comprise the sequences set forth in SEQ ID NO: 78 and 39, respectively;
the VH region and the VL region comprise the sequences set forth in SEQ ID NO: 80 and 39, respectively;
the VH region and the VL region comprise the sequences set forth in SEQ ID NO: 82 and 39, respectively;
the VH region and the VL region comprise the sequences set forth in SEQ ID NO: 84 and 39, respectively;
the VH region and the VL region comprise the sequences set forth in SEQ ID NO: 86 and 39, respectively;
the VH region and the VL region comprise the sequences set forth in SEQ ID NO: 159 and 43, respectively;
the VH region and the VL region comprise the sequences set forth in SEQ ID NO: 195 and 39, respectively;
the VH region and the VL region comprise the sequences set forth in SEQ ID NO: 41 and 37, respectively;
the VH region and the VL region comprise the sequences set forth in SEQ ID NO: 64 and 37, respectively;
the VH region and the VL region comprise the sequences set forth in SEQ ID NO: 65 and 37, respectively;
the VH region and the VL region comprise the sequences set forth in SEQ ID NO: 66 and 37, respectively;
the VH region and the VL region comprise the sequences set forth in SEQ ID NO: 67 and 37, respectively;
the VH region and the VL region comprise the sequences set forth in SEQ ID NO: 69 and 37, respectively;
the VH region and the VL region comprise the sequences set forth in SEQ ID NO: 71 and 37, respectively;
the VH region and the VL region comprise the sequences set forth in SEQ ID NO: 75 and 37, respectively;
the VH region and the VL region comprise the sequences set forth in SEQ ID NO: 76 and 37, respectively;
the VH region and the VL region comprise the sequences set forth in SEQ ID NO: 77 and 37, respectively;
the VH region and the VL region comprise the sequences set forth in SEQ ID NO: 79 and 37, respectively;
the VH region and the VL region comprise the sequences set forth in SEQ ID NO: 81 and 37, respectively;
the VH region and the VL region comprise the sequences set forth in SEQ ID NO: 83, and 37, respectively;
the VH region and the VL region comprise the sequences set forth in SEQ ID NO: 85 and 37, respectively;
the VH region and the VL region comprise the sequences set forth in SEQ ID NO: 87 and 37, respectively;
the VH region and the VL region comprise the sequences set forth in SEQ ID NO: 90 and 37, respectively;
the VH region and the VL region comprise the sequences set forth in SEQ ID NO: 91 and 37, respectively;
the VH region and the VL region comprise the sequences set forth in SEQ ID NO: 92 and 37, respectively;
the VH region and the VL region comprise the sequences set forth in SEQ ID NO: 93 and 37, respectively;
the VH region and the VL region comprise the sequences set forth in SEQ ID NO: 94 and 37, respectively;
the VH region and the VL region comprise the sequences set forth in SEQ ID NO: 95 and 37, respectively;
the VH region and the VL region comprise the sequences set forth in SEQ ID NO: 96 and 37, respectively;
the VH region and the VL region comprise the sequences set forth in SEQ ID NO: 97 and 37, respectively;
the VH region and the VL region comprise the sequences set forth in SEQ ID NO: 98 and 37, respectively;
the VH region and the VL region comprise the sequences set forth in SEQ ID NO: 36 and 39, respectively;
the VH region and the VL region comprise the sequences set forth in SEQ ID NO: 36 and 99, respectively;
the VH region and the VL region comprise the sequences set forth in SEQ ID NO: 36 and 43, respectively;
the VH region and the VL region comprise the sequences set forth in SEQ ID NO: 36 and 100, respectively;
the VH region and the VL region comprise the sequences set forth in SEQ ID NO: 36 and 101, respectively;
the VH region and the VL region comprise the sequences set forth in SEQ ID NO: 42 and 37, respectively;
the VH region and the VL region comprise the sequences set forth in SEQ ID NO: 38 and 37, respectively;
the VH region and the VL region comprise the sequences set forth in SEQ ID NO: 40 and 37, respectively.

29. The bispecific antibody of any one of claims 20-28, wherein in the second binding domain:
the VH region comprising a sequence of the consensus sequence QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTX₈X₉YX₁₀X₁₁GX₁₂X₁₃VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is R or G; X₉ is Y or H; X₁₀ is Q or S; Xn₁₁ is Y or H; X₁₂ is I or L; and X₁₃ is N or D; (SEQ ID NO: 116); and
the VL region comprising a sequence of the consensus sequence set forth in AIQLTQSPSSLSASVGDRVTITCRASQGISSALAWYQQKPGKAPKLLIYDASSLEX₁GV PSRFSGSGSGTDFTLTISSLQPEDFATYYCQQFX₂SYPRTFGX₃GTKVEIK, wherein X₁ is N or S; X₂ is K or N; and X₃ is Q or C; (SEQ ID NO: 117).

30. The bispecific antibody of any one of claims 20-29, wherein in the second binding domain:
the VH region comprising a sequence of the consensus sequence QVQLVESGGGVVQPGX₁SLRLSCAASGFTFSX₂YGIHWVRQAPGKX₃LEWVAAIWYD X₄SKX₅YYADSVKGRFTISRDNSX₆NTLX₇LQMNSLRAEDTAVYYCARSNYDILTRYY X₈X₉GX₁₀X₁₁VWGQGTTVTVSS, wherein X₁ is R, G, or E; X₂ is R or N; X₃ is G or C; X₄ is G or A; X₅ is K, T, or D; X₆ is K, E, or S; X₇ is Y or D; X₈ is Q or S; X₉ is Y or H; X₁₀ is I or L; and Xn₁₁ is N or D; (SEQ ID NO: 192); and
the VL region comprising a sequence of the consensus sequence set forth in AIQLTQSPSSLSASVGDRVTITCRASQGISSALAWYQQKPGKAPKLLIYDASSLESGVP SRFSGSGSGTDFTLTISSLQPEDFATYYCQQFX₁SYPRTFGX₂GTKVEIK, wherein X₁=K or N X₂=Q or C; (SEQ ID NO: 193).

31. The bispecific antibody of any one of claims 20-30, wherein in the second binding domain, the VH comprises one or more amino acid residues selected from the group consisting of: an asparagine (N) at position 31, an alanine (A) at position 55, a tyrosine (Y) at position 80, a glycine (G) at position 106, a histidine (H) at position 107, a serine (S) at position 109, a histidine (H) at position 110, a leucine (L) at position 112, and an aspartic acid (D) at position 113 or any combination thereof, wherein the numbering is relative to SEQ ID NO: 36.

32. The bispecific antibody of any one of claims 20-31, wherein, in the second binding domain, the VH comprises:
(a) amino acid residues asparagine (N) at position 31, alanine (A) at position 55, and tyrosine (Y) at position 80, wherein the numbering is relative to SEQ ID NO: 36;
(b) alanine (A) at position 55, tyrosine (Y) at position 80, leucine (L) at position 112, and aspartic acid (D) at position 113, wherein the numbering is relative to SEQ ID NO: 36; or
(c) alanine (A) at position 55, tyrosine (Y) at position 80, serine (S) at position 109, and histidine (H) at position 110, wherein the numbering is relative to SEQ ID NO: 36;

33. The bispecific antibody of any one of claims 20-32, wherein, in the second binding domain, the VL region comprises one or more amino acid residues selected from the group consisting of: a serine (S) at position 56, an asparagine (N) at position 92, and a cysteine (C) at position 100, an asparagine (N) at position 108, or any combination thereof in relation to SEQ ID NO: 37.

34. The bispecific antibody of any of claims 20-33, wherein, in the second binding domain, the VL region comprises:
(a) serine (S) at position 56, wherein the numbering is relative to SEQ ID NO: 36; or
(b) serine (S) at position 56 and asparagine (N) at position 92, wherein the numbering is relative to SEQ ID NO: 37.

35. The bispecific antibody of any one of claims 20-34, wherein, in the second binding domain, the VH comprises one or more amino acid residues selected from the group consisting of: a glycine (G) at position 16, a glutamic acid (E) at position 16, a threonine (T) at position 58, an aspartic acid (D) at position 58, a glutamic acid (E) at position 76, a serine (S) at position 76, or any combination thereof in relation to SEQ ID NO: 36.

36. The bispecific antibody of any of claims 1-35, wherein the second binding domain is an antigen binding antibody fragment.

37. The bispecific antibody of any of claims 1-35, wherein the second binding domain is a scFv, is a Fab, or is an Fv.

38. The bispecific antibody of claim 36 or claim 37, wherein the second binding domain is an scFv.

39. The bispecific antibody of any one of claims 37-38, wherein the scFv comprises a VH-linker-VL arrangement.

40. The bispecific antibody of any one of claims 37-39, wherein the scFv comprises a VL-linker-VH arrangement.

41. The bispecific antibody of claim 39 or claim 40, wherein the scFv comprises a peptide linker comprising (Gly4Ser)3 (SEQ ID NO: 162).

42. The bispecific antibody of any of claims 1-41, wherein the second binding domain is an scFv that is a disulfide-stabilized scFv.

43. The bispecific antibody of claim 39, wherein, in the second binding domain, the VH region comprises cysteine (C) at position 44 and the VL region comprises a cysteine (C) at position 100 in relation to SEQ ID NO: 36.

44. The bispecific antibody of any one of claims 1-43, wherein, in the second binding domain, the VH region comprises the CDR-H1, the CDR-H2, and the CDR-H3 contained within any one of SEQ ID NOs: 36, 38, 40, 42, 22, 68, 70, 72, 74, 78, 80, 82, 84, 86, 159 or 195, and the VL region comprises the CDR-L1, the CDR-L2, and the CDR-L3 contained within any one of SEQ ID NOs: 27, 37, 39, 43, or 73.

45. The bispecific antibody of any one of claims 1-44, wherein, in the second binding domain:
the CDR-H1 comprises the sequence set forth in any one of SEQ ID NOs: 19, or 109, the CDR-H2 comprises the sequence set forth in any one of SEQ ID NOs: 20, 107, 110, or 112, and the CDR-H3 comprises the sequence set forth in any one of SEQ ID NOs: 21, 108, 111, or 113; and
the CDR-L1 comprises the sequence set forth in SEQ ID NOs: 24, the CDR-L2 comprises the sequence set forth in any one of SEQ ID NOs: 25 or 114, and the CDR-L3 comprises the sequence set forth in any one of SEQ ID NOs: 26 or 115.

46. The bispecific antibody of any one of claims 1-45, wherein in the second binding domain:
the VH region comprises the CDR-H1, the CDR-H2, and the CDR-H3 contained within SEQ ID NO: 36, and the VL region comprises the CDR-L1, the CDR-L2, and the CDR-L3 contained within SEQ ID NO: 37;
the VH region comprises the CDR-H1, the CDR-H2, and the CDR-H3 contained within SEQ ID NO: 38, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 40, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 42, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 22, and the VL region comprises the CDR-L1, the CDR-L2, and the CDR-L3 contained within SEQ ID NO: 27;
the VH region comprises the CDR-H1, the CDR-H2, and the CDR-H3 contained within SEQ ID NO: 68, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 70, and the VL region comprises the CDR-L1, the CDR-L2, and the CDR-L3 contained within SEQ ID NO: 43;
the VH region comprises the CDR-H1, the CDR-H2, and the CDR-H3 contained within SEQ ID NO: 72, and the VL region comprises the CDR-L1, the CDR-L2, and the CDR-L3 contained within SEQ ID NO: 73;
the VH region comprises the CDR-H1, the CDR-H2, and the CDR-H3 contained within SEQ ID NO: 74, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 78, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 80, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 82, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 84, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 86, and the VL region comprises the CDR-L1, the CDR-L2, and the CDR-L3 contained within SEQ ID NO: 39;
the VH region comprises the CDR-H1, the CDR-H2, and the CDR-H3 contained within SEQ ID NO: 159, and the VL region comprises the CDR-L1, the CDR-L2, and the CDR-L3 contained within SEQ ID NO: 43; or
the VH region comprises the CDR-H1, the CDR-H2, and the CDR-H3 contained within SEQ ID NO: 195, and the VL region comprises the CDR-L1, the CDR-L2, and the CDR-L3 contained within SEQ ID NO: 39.

47. The bispecific antibody of any one of claims 1-46, wherein in the second binding domain:
the VH region comprises the CDR-H1, the CDR-H2, and the CDR-H3 comprising the sequence set forth in SEQ ID NOS: 19, 107, and 108, respectively, and the VL region comprises the CDR-L1, the CDR-L2, and the CDR-L3 comprising the sequence set forth in SEQ ID NOS: 24, 114, and 26, respectively;
the VH region comprises the CDR-H1, the CDR-H2, and the CDR-H3 comprising the sequence set forth in SEQ ID NOS: 109, 110, and 108, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 110, and 21, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 110, and 108, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 20, and 21, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 20, and 111, respectively, and the VL region comprises the CDR-L1, the CDR-L2, and the CDR-L3 comprising the sequence set forth in SEQ ID NOS: 24, 25, and 115, respectively;
the VH region comprises the CDR-H1, the CDR-H2, and the CDR-H3 comprising the sequence set forth in SEQ ID NOS: 19, 112, and 21, respectively, and the VL region comprises the CDR-L1, the CDR-L2, and the CDR-L3 comprising the sequence set forth in SEQ ID NOS: 24, 25, and 26, respectively;
the VH region comprises the CDR-H1, the CDR-H2, and the CDR-H3 comprising the sequence set forth in SEQ ID NOS: 19, 20, and 113, respectively, and the VL region comprises the CDR-L1, the CDR-L2, and the CDR-L3 comprising the sequence set forth in SEQ ID NOS: 24, 25, and 115, respectively.

48. The bispecific antibody of any one of claims 1-47, wherein in the second binding domain:
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 36, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 37;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 38, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 40, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 42, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 22, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 27;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 68, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 70, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 72, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 73;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 74, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 78, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 80, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 82, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 84, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 86, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 159, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 43; or
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 195, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 39.

49. The bispecific antibody of any one of claims 1-48, wherein in the second binding domain:
the VH region and the VL region comprise the sequences set forth in SEQ ID NO: 36 and 37, respectively;
the VH region and the VL region comprise the sequences set forth in SEQ ID NO: 38 and 39, respectively;
the VH region and the VL region comprise the sequences set forth in SEQ ID NO: 40 and 39, respectively;
the VH region and the VL region comprise the sequences set forth in SEQ ID NO: 42 and 43, respectively;
the VH region and the VL region comprise the sequences set forth in SEQ ID NO: 22 and 27, respectively;
the VH region and the VL region comprise the sequences set forth in SEQ ID NO: 68 and 39, respectively;
the VH region and the VL region comprise the sequences set forth in SEQ ID NO: 70 and 43, respectively;
the VH region and the VL region comprise the sequences set forth in SEQ ID NO: 72 and 73, respectively;
the VH region and the VL region comprise the sequences set forth in SEQ ID NO: 74 and 39, respectively;
the VH region and the VL region comprise the sequences set forth in SEQ ID NO: 78 and 39, respectively;
the VH region and the VL region comprise the sequences set forth in SEQ ID NO: 80 and 39, respectively;
the VH region and the VL region comprise the sequences set forth in SEQ ID NO: 82 and 39, respectively;
the VH region and the VL region comprise the sequences set forth in SEQ ID NO: 84 and 39, respectively;
the VH region and the VL region comprise the sequences set forth in SEQ ID NO: 86 and 39, respectively;
the VH region and the VL region comprise the sequences set forth in SEQ ID NO: 159 and 43, respectively; or
the VH region and the VL region comprise the sequences set forth in SEQ ID NO: 195 and 39, respectively.

50. The bispecific antibody of any one of claims 1-49, wherein the second binding domain is an scFv comprising:
(a) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 246;
(b) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 247;
(c) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 250; or
(d) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 251.

51. The bispecific antibody of any one of claims 1-50, wherein the second binding domain is an scFv comprising:
(a) the amino acid sequence of SEQ ID NO: 246;
(b) the amino acid sequence of SEQ ID NO: 247;
(c) the amino acid sequence of SEQ ID NO: 250; or
(d) the amino acid sequence of SEQ ID NO: 251.

52. The bispecific antibody of any one of claims 1-51, wherein in the second binding domain, the VH region comprises the CDR-H1, the CDR-H2, and the CDR-H3 comprising the sequence set forth in SEQ ID NOs: 19, 20, and 21, respectively, and the VL region comprises the CDR-L1, the CDR-L2, and the CDR-L3 comprising the sequence set forth in SEQ ID NOS: 24, 25, and 26, respectively.

53. The bispecific antibody of any one of claims 1-52, wherein in the second binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 22, and the VL region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 27.

54. The bispecific antibody of any one of claims 1-53 wherein in the second binding domain, the VH region and the VL region comprises the sequence or are the sequences set forth in SEQ ID NO: 22 and 27, respectively.

55. The bispecific antibody of any one of claims 1-52, wherein in the second binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 68, and the VL region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 39.

56. The bispecific antibody of any one of claims 1-51, and 55, wherein in the second binding domain, the VH region and the VL region comprise the sequences or are the sequences set forth in SEQ ID NO: 68 and 39, respectively.

57. The bispecific antibody of any one of claims 1-51, wherein in the second binding domain, the VH region comprises the CDR-H1, the CDR-H2, and the CDR-H3 comprising the sequence set forth in SEQ ID NOs: 19, 20, and 111, respectively, and the VL region comprises the CDR-L1, the CDR-L2, and the CDR-L3 comprising the sequence set forth in SEQ ID NOS: 24, 25, and 115, respectively.

58. The bispecific antibody of any one of claims 1-51 and 57, wherein in the second binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 70, and the VL region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 43.

59. The bispecific antibody of any one of claims 1-51, 57 and 58, wherein in the CD3 binding domain, the VH region and the VL region comprise the sequences or are the sequences set forth in SEQ ID NO: 70 and 43, respectively.

60. The bispecific antibody of any one of claims 1-51 and 59, wherein in the second binding domain, the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 72, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 73.

61. The bispecific antibody of any one of claims 1-51, 59 and 60, wherein in the second binding domain, the VH region and the VL region comprise the sequences or are the sequences set forth in SEQ ID NO: 72 and 73, respectively.

62. The bispecific antibody of any one of claims 1-61, wherein:
the first binding domain comprises the VH set forth in any one of SEQ ID NOs: 12 or 88 and the VL set forth in any one of SEQ ID NO: 17 or 89; and
the second binding domain comprises the VH set forth in any one of SEQ ID NOs: 22, 68, 72, or 70 and the VL set forth in any one of SEQ ID NO: 27, 39, 73, or 43.

63. The bispecific antibody of any of claims 1-62, wherein:
the first binding domain comprises the VH set forth in SEQ ID NO: 12 and the VL set forth in SEQ ID NO: 17; and
the second binding domain comprises the VH set forth in SEQ ID NO: 22 and the VL set forth in SEQ ID NO: 27.

64. The bispecific antibody of any of claims 1-62, wherein:
the first binding domain comprises the VH set forth in SEQ ID NO: 12 and the VL set forth in SEQ ID NO: 17; and
the second binding domain comprises the VH set forth in SEQ ID NO: 68 and the VL set forth in SEQ ID NO: 39.

65. The bispecific antibody of any of claims 1-62, wherein:
the first binding domain comprises the VH set forth in SEQ ID NO: 12 and the VL set forth in SEQ ID NO: 17; and
the CD3 binding domain comprises the VH set forth in SEQ ID NO: 72 and the VL set forth in SEQ ID NO: 73.

66. The bispecific antibody of any of claims 1-62, wherein:
the first binding domain comprises the VH set forth in SEQ ID NO: 12 and the VL set forth in SEQ ID NO: 17; and
the second binding domain comprises the VH set forth in SEQ ID NO: 70 and the VL set forth in SEQ ID NO: 43.

67. The bispecific antibody of any of claims 1-62, wherein:
the first binding domain comprises the VH set forth in SEQ ID NO: 88 and the VL set forth in SEQ ID NO: 89; and
the second binding domain comprises the VH set forth in SEQ ID NO: 22 and the VL set forth in SEQ ID NO: 27.

68. The bispecific antibody of any of claims 1-62, wherein:
the first binding domain comprises the VH set forth in SEQ ID NO: 88 and the VL set forth in SEQ ID NO: 89; and
the second binding domain comprises the VH set forth in SEQ ID NO: 68 and the VL set forth in SEQ ID NO: 39.

69. The bispecific antibody of any of claims 1-62, wherein:
the first binding domain comprises the VH set forth in SEQ ID NO: 88 and the VL set forth in SEQ ID NO: 89; and
the second binding domain comprises the VH set forth in SEQ ID NO: 72 and the VL set forth in SEQ ID NO: 73.

70. The bispecific antibody of any of claims 1-62, wherein:
the first binding domain comprises the VH set forth in SEQ ID NO: 88 and the VL set forth in SEQ ID NO: 89; and
the second binding domain comprises the VH set forth in SEQ ID NO: 70 and the VL set forth in SEQ ID NO: 43.

71. The bispecific antibody of any of claims 1-70, wherein the second binding domain has a KD of 5 to 75 nM to CD3.

72. The bispecific antibody of any of claims 1-71, wherein the second binding domain has a KD of 10 to 70 nM to CD3.

73. The bispecific antibody of any one of claims 1-72, wherein the first binding domain and the second binding domain are independently selected from a scFv and a Fab.

74. The bispecific antibody of any one of claims 1-73, wherein the first binding domain and/or the second binding domain is a scFv.

75. The bispecific antibody of any one of claims 1-74, wherein the first and the second binding domain are both an scFv.

76. The bispecific antibody of any one of claims 1-74, wherein the first binding domain is a scFv and the second binding domain is a Fab.

77. The bispecific antibody of any one of claims 1-76, wherein the first binding domain comprises, from N- to C-terminal order: the VH region and the VL region, optionally separated by a linker.

78. The bispecific antibody of any one of claims 1-77, wherein the first binding domain is a scFv that is disulfide-stabilized such that the VH region and VL region are covalently linked by a disulfide bond, optionally wherein the VH region comprises a cysteine residue at framework region 2 position 44 (Kabat numbering) and the VL region comprises a cysteine residue at framework region 4 position 100 (Kabat numbering).

79. The bispecific antibody of any one of claims 1-16, 20-66 and 71-78, wherein the first binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 245.

80. The bispecific antibody of any one of claims 1-16, 20-66 and 71-79, wherein the first binding domain comprises the amino acid sequence of SEQ ID NO: 245.

81. The bispecific antibody of any one of claims 1-16, 20-62 and 67-78, wherein the first binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 248.

82. The bispecific antibody of any one of claims 1-16, 20-62, 67-78 and 81, wherein the first binding domain comprises the amino acid sequence of SEQ ID NO: 248.

83. The bispecific antibody of any one of claims 1-82, wherein the second binding domain comprises, from N- to C-terminal order: the VH region and the VL region, optionally separated by a linker.

84. The bispecific antibody of any one of claims 1-83, wherein the second binding domain is a scFv that is disulfide-stabilized such that the VH region and VL region are covalently linked by a disulfide bond, optionally wherein the VH region comprises a cysteine residue at framework region 2 position 44 (Kabat numbering) and the VL region comprises a cysteine residue at framework region 4 position 100 (Kabat numbering).

85. The bispecific antibody of any one of claims 1-54, 62-63, 67, 71-75 and 77-84, wherein the second binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 246.

86. The bispecific antibody of any one of claims 1-54, 62-63, 67, 71-75 and 77-84, wherein the second binding domain comprises the amino acid sequence of SEQ ID NO: 246.

87. The bispecific antibody of any one of claims 1-49, 57, 60, 61, 62, 65, 69, 71-75 and 77-84, wherein the second binding domain comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 247.

88. The bispecific antibody of any one of claims 1-49, 57, 60, 61, 62, 65, 69, 71-75 and 77-84 and 87, wherein the second binding domain comprises the amino acid sequence of SEQ ID NO: 247.

89. The bispecific antibody of any of claims 1-88, wherein the antibody comprises a constant region from an IgG1 or IgG2.

90. The bispecific antibody of claim 89, wherein the constant region is a human IgG1 heavy chain constant region.

91. The bispecific antibody of any one of claims 1-89, wherein the antibody comprises an Fc region comprising a first and second Fc polypeptide each comprising a hinge-CH2-CH3.

92. The bispecific antibody of any one of claims 1-75 and 77-91, wherein the first binding domain and the second binding domain are together present on two polypeptide chains.

93. The bispecific antibody of any one of claims 1-75 and 77-92, comprising a first heavy chain and a second heavy chain, wherein:
(a) the first heavy chain comprises (i) the first binding domain that is an scFv comprising the VH region and the VL region and (ii) a first Fc polypeptide comprising a hinge-CH2-CH3; and
(b) the second heavy chain comprises (i) the second binding domain that is an scFv comprising the VH region and the VL region and (ii) a second Fc polypeptide comprising a hinge-CH2-CH3.

94. The bispecific antibody of any one of claims 1-75 and 77-91, wherein the first binding domain and the second binding domain are together present on three polypeptide chains.

95. The bispecific antibody of any one of claims 1-75, 77-91 and 94, comprising a first heavy chain, a second heavy chain, and a light chain, wherein:
(a) the first heavy chain comprises (i) the first binding domain that is an scFv comprising the VH region and the VL region and (ii) a first Fc polypeptide comprising a hinge-CH2-CH3;
(b) the second heavy chain comprises (i) the heavy chain of the second binding domain that is the heavy chain of a Fab comprising the VH region and CH1, (ii) and a second Fc polypeptide comprising a hinge-CH2-CH3; and
(c) a light chain comprises the light chain of the second binding domain that is the light chain of the Fab comprising the VL region and the constant light chain (CL).

96. The bispecific antibody of any one of claims 1-95, wherein the first binding domain and the second binding domain are each N-terminal to the Fc region.

97. The bispecific antibody of any one of claims 91-96, wherein the Fc region is a heterodimeric Fc region.

98. The bispecific antibody of any one of claims 91-97, wherein each of the first and second polypeptide comprises one or more amino acid substitutions in a wild-type Fc polypeptide region to effect heterodimer formation between the first polypeptide and the second polypeptide.

99. The bispecific antibody of claim 98, wherein the wild-type Fc region is an IgG1 Fc region or an IgG2 Fc region, optionally wherein the wild-type Fc region is a wildtype IgG1 Fc region comprising the amino acid sequence set forth in SEQ ID NO: 253.

100. The bispecific antibody of claim 98 or claim 99, wherein the one more amino acid substitutions are a knob-into-hole modification, a charge mutation to reduce or prevent self-association due to charge repulsion, or disulfide bonding pairing mutation, or combinations thereof.

101. The bispecific antibody of any of claims 98-100, wherein the one or more amino acid substitutions are a knob-into-hole modification and disulfide bond pairing mutation.

102. The bispecific antibody of any of claims 98-101, wherein the one or more amino acid substitutions are:
(i) amino acid substitution T366W in the first polypeptide of the heterodimeric Fc and amino acid substitutions T366S, L368A and Y407V in the second polypeptide of the heterodimeric Fc; and/or
(ii) amino acid substitution S354C on the first polypeptide of the heterodimeric Fc and amino acid substitution Y349C on the second polypeptide of the heterodimeric Fc.

103. The bispecific antibody of any of claims 98-102, wherein the heterodimeric Fc region comprises one or more amino acid substitutions to reduce binding affinity to an Fc receptor and/or to reduce effector function, optionally as compared to a wild-type IgG1 Fc region.

104. The bispecific antibody of claim 99, wherein the wild-type Fc region is a wild-type IgG1 Fc region comprising the amino acid sequence set forth in SEQ ID NO: 253.

105. The bispecific antibody of claim 103, wherein the one or more amino acid substitutions are selected from L234A, L234V, L235A, L235E, G237A, D265S, S267K, R292C, N297G, V302C, and P329G by EU numbering.

106. The bispecific antibody of claim 105, wherein the one or more amino acid substitution comprises L234A and L235A.

107. The bispecific antibody of any of claims 104-106, wherein the one or more amino acid substitution comprises L234A, L235A, and D265S.

108. The bispecific antibody of any one of claims 91-107, wherein the first and second Fc polypeptide is of the human IgG1 G1m1,17 allotype.

109. The bispecific antibody of any one of claims 91-108, wherein the first and second Fc polypeptide comprise the amino acid substitutions D356E and L358M.

110. The bispecific antibody of any one of claims 91-109, wherien:
the first Fc polypeptide is an IgG1 Fc comprising amino acid substitutions L234A, L235A, D265S, S354C and T366W, and the second Fc polypeptide is an IgG1 Fc comprising amino acid substitutions L234A, L235A, D265S, Y349C, T366S, L368A and Y407V; or
the first Fc polypeptide is an IgG1 Fc comprising amino acid substitutions L234A, L235A, G237A, S354C and T366W, and the second Fc polypeptide is an IgG1 Fc comprising amino acid substitutions L234A, L235E, Y349C, T366S, L368A and Y407V.

111. The bispecific antibody of any of claims 91-110, wherein:
the first Fc polypeptide comprises an amino acid sequence having at least at or about 85%, 86%, 87%, 88%, 89%, 90%, 91%, 92%, 93%, 94%, 95%, 96%, 97%, 98% or 99% sequence identity SEQ ID NO:163 or 165; and
the second Fc polypeptide comprises an amino acid sequence having at least at or about 85%, 86%, 87%, 88%, 89%, 90%, 91%, 92%, 93%, 94%, 95%, 96%, 97%, 98% or 99% sequence identity SEQ ID NO: 164 or 166.

112. The bispecific antibody of any of claims 91-111, wherein:
the first Fc polypeptide comprises the amino acid sequence set forth in SEQ ID NO: 164 or 166; and
the second Fc polypeptide comprises the amino acid sequence set forth in SEQ ID NO: 163 or 165.

113. The bispecific antibody of any of claims 91-112, wherein the first Fc polypeptide of the heterodimeric Fc region comprises the amino acid sequence set forth in SEQ ID NO: 164 and the second Fc polypeptide of the heterodimeric Fc region comprises the amino acid sequence set forth in SEQ ID NO: 163.

114. The bispecific antibody of any of claims 91-113, wherein the first Fc polypeptide of the heterodimeric Fc region comprises the amino acid sequence set forth in SEQ ID NO: 166 and the second Fc polypeptide of the heterodimeric Fc region comprises the amino acid sequence set forth in SEQ ID NO:165.

115. The bispecific antibody of claim 103-109, wherein the one or more amino acid substitutions are selected from P238K.

116. The bispecific antibody of any one of claims 91-109 and 115, wherien the first Fc polypeptide is an IgG2 Fc comprising amino acid substitutions P238K, D356E, L358M, Y349C, T366S, L368A and Y407V, and the second Fc polypeptide is an IgG2 Fc comprising amino acid substitutions P238K, D356E, L358M, S354C and T366W.

117. The bispecific antibody of any of claims 91-109, 115 and 116, wherein:
the first Fc polypeptide comprises an amino acid sequence having at least at or about 85%, 86%, 87%, 88%, 89%, 90%, 91%, 92%, 93%, 94%, 95%, 96%, 97%, 98% or 99% sequence identity SEQ ID NO:168; and
the second Fc polypeptide comprises an amino acid sequence having at least at or about 85%, 86%, 87%, 88%, 89%, 90%, 91%, 92%, 93%, 94%, 95%, 96%, 97%, 98% or 99% sequence identity SEQ ID NO: 167.

118. The bispecific antibody of any of claims 91-109 and 115-117, wherein the first Fc polypeptide of the heterodimeric Fc region comprises the amino acid sequence set forth in SEQ ID NO: 168 and the second Fc polypeptide of the heterodimeric Fc region comprises the amino acid sequence set forth in SEQ ID NO:167.

119. The bispecific antibody of any of claims 1-118, wherein the bispecific antibody is capable of binding CD19 on B cells and CD3 on T cells.

120. The bispecific antibody of any of claims 1-119, wherein the bispecific antibody results in B cell and/or plasma blast depletion of greater than 90% in the lymph node, bone marrow, and/or blood as determined by an in vitro cell depletion assay.

121. A bispecific antibody, comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and
(b) a second heavy chain polypeptide comprising from N- to C-terminal order: a VH region comprising the sequence set forth in SEQ ID NO: 22, a linker of SEQ ID NO: 162, a VL region comprising the sequence set forth in SEQ ID NO: 27, and a Fc region comprising the sequence set forth in SEQ ID NO:165.

122. The bispecific antibody of claim 121, wherein:
the first heavy chain polypeptide comprises from N-to C-terminal order the scFv comprising the sequence set forth in SEQ ID NO:245 and the Fc region comprising the sequence set forth in SEQ ID NO:166; and
the second heavy chain polypeptide comprises from N-to C-terminal order the scFv comprising the sequence set forth in SEQ ID NO:246 and the Fc region comprising the sequence set forth in SEQ ID NO: 165.

123. The bispecific antibody of claim 121 or claim 122, wherein the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 172; and
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 173.

124. The bispecific antibody of any one of claims 121-123, wherein:
(a) the first heavy chain polypeptide comprises the amino acid sequence of SEQ ID NO: 172; and
(b) the second heavy chain polypeptide comprises the amino acid sequence of SEQ ID NO: 173.

125. A bispecific antibody, comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and
(b) a second heavy chain polypeptide comprising from N- to C-terminal order: a Fab comprising a VH region comprising the sequence set forth in SEQ ID NO: 68 and an IgG1 CH1 of SEQ ID NO: 169, and a Fc region comprising the sequence set forth in SEQ ID NO:165; and
(c) a light chain comprises a VL region comprising the sequence set forth in SEQ ID NO: 39 and a CL comprising the sequence set forth in SEQ ID NO: 171.

126. The bispecific antibody of claim 125, wherein:
the first heavy chain polypeptide comprises from N-to C-terminal order the scFv comprising the sequence set forth in SEQ ID NO:245 and the Fc region comprising the sequence set forth in SEQ ID NO:166.

127. The bispecific antibody of claim 125 and 126, wherein the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 174;
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 175; and
(c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 176.

128. The bispecific antibody of any one of claims 125-127, wherein:
(a) the first heavy chain polypeptide comprises the amino acid sequence of SEQ ID NO: 174;
(b) the second heavy chain polypeptide comprises the amino acid sequence of SEQ ID NO: 175; and
(c) the light chain comprises the amino acid sequence of SEQ ID NO: 176.

129. A bispecific antibody, comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and
(b) a second heavy chain polypeptide comprising from N- to C-terminal order: a VH region comprising the sequence set forth in SEQ ID NO: 72, a linker of SEQ ID NO: 162, a VL region comprising the sequence set forth in SEQ ID NO: 73, and a Fc region comprising the sequence set forth in SEQ ID NO:165.

130. The bispecific antibody of claim 129, wherein:
the first heavy chain polypeptide comprises from N-to C-terminal order the scFv comprising the sequence set forth in SEQ ID NO:245 and the Fc region comprising the sequence set forth in SEQ ID NO:166; and
the second heavy chain polypeptide comprises from N-to C-terminal order the scFv comprising the sequence set forth in SEQ ID NO:247 and the Fc region comprising the sequence set forth in SEQ ID NO: 165.

131. The bispecific antibody of claim 129 or claim 130, wherein the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 177; and
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 178.

132. The bispecific antibody of any one of claims 129-131, wherein:
(a) the first heavy chain polypeptide comprises the amino acid sequence of SEQ ID NO: 177; and
(b) the second heavy chain polypeptide comprises the amino acid sequence of SEQ ID NO: 178.

133. A bispecific antibody, comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 166; and
(b) a second heavy chain polypeptide comprising from N- to C-terminal order: a Fab comprising a VH region comprising the sequence set forth in SEQ ID NO: 70 and an IgG1 CH1 of SEQ ID NO: 169, and a Fc region comprising the sequence set forth in SEQ ID NO:165; and
(c) a light chain comprises a VL region comprising the sequence set forth in SEQ ID NO: 43 and a CL comprising the sequence set forth in SEQ ID NO: 171.

134. The bispecific antibody of claim 133, wherein:
the first heavy chain polypeptide comprises from N-to C-terminal order the scFv comprising the sequence set forth in SEQ ID NO:245 and the Fc region comprising the sequence set forth in SEQ ID NO:166.

135. The bispecific antibody of claim 133 and 134, wherein the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 179;
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 180; and
(c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 181.

136. The bispecific antibody of any one of claims 133-135, wherein:
(a) the first heavy chain polypeptide comprises the amino acid sequence of SEQ ID NO: 179;
(b) the second heavy chain polypeptide comprises the amino acid sequence of SEQ ID NO: 180; and
(c) the light chain comprises the amino acid sequence of SEQ ID NO: 181.

137. A bispecific antibody, comprising:
(a) a first heavy chain polypeptide comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and
(b) a second heavy chain polypeptide comprising from N- to C-terminal order: a VH region comprising the sequence set forth in SEQ ID NO: 22, a linker of SEQ ID NO: 162, a VL region comprising the sequence set forth in SEQ ID NO: 27, and a Fc region comprising the sequence set forth in SEQ ID NO:165.

138. The bispecific antibody of claim 137, wherein:
the first heavy chain polypeptide comprises from N-to C-terminal order the scFv comprising the sequence set forth in SEQ ID NO:248 and the Fc region comprising the sequence set forth in SEQ ID NO:166; and
the second heavy chain polypeptide comprises from N-to C-terminal order the scFv comprising the sequence set forth in SEQ ID NO:246 and the Fc region comprising the sequence set forth in SEQ ID NO: 165.

139. The bispecific antibody of claim 137 or claim 138, wherein the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 182; and
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 183.

140. The bispecific antibody of any one of claims 137-139, wherein:
(a) the first heavy chain polypeptide comprises the amino acid sequence of SEQ ID NO: 182; and
(b) the second heavy chain polypeptide comprises the amino acid sequence of SEQ ID NO: 183.

141. A bispecific antibody, comprising:
(a) a first heavy chain polypeptide comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and
(b) a second heavy chain polypeptide comprising from N- to C-terminal order: a Fab comprising a VH region comprising the sequence set forth in SEQ ID NO: 68 and an IgG1 CH1 of SEQ ID NO: 169, and a Fc region comprising the sequence set forth in SEQ ID NO:165; and
(c) a light chain comprises a VL region comprising the sequence set forth in SEQ ID NO: 39 and a CL comprising the sequence set forth in SEQ ID NO: 171.

142. The bispecific antibody of claim 141, wherein:
the first heavy chain polypeptide comprises from N-to C-terminal order the scFv comprising the sequence set forth in SEQ ID NO:248 and the Fc region comprising the sequence set forth in SEQ ID NO:166.

143. The bispecific antibody of claim 141 and 142, wherein the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 184;
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 185; and
(c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 186.

144. The bispecific antibody of any one of claims 141-143, wherein:
(a) the first heavy chain polypeptide comprises the amino acid sequence of SEQ ID NO: 184;
(b) the second heavy chain polypeptide comprises the amino acid sequence of SEQ ID NO: 185; and
(c) the light chain comprises the amino acid sequence of SEQ ID NO: 186.

145. A bispecific antibody, comprising:
(a) a first heavy chain polypeptide comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and
(b) a second heavy chain polypeptide comprising from N- to C-terminal order: a VH region comprising the sequence set forth in SEQ ID NO: 72, a linker of SEQ ID NO: 162, a VL region comprising the sequence set forth in SEQ ID NO: 73, and a Fc region comprising the sequence set forth in SEQ ID NO:165.

146. The bispecific antibody of claim 145, wherein:
the first heavy chain polypeptide comprises from N-to C-terminal order the scFv comprising the sequence set forth in SEQ ID NO:248 and the Fc region comprising the sequence set forth in SEQ ID NO:166; and
the second heavy chain polypeptide comprises from N-to C-terminal order the scFv comprising the sequence set forth in SEQ ID NO:247 and the Fc region comprising the sequence set forth in SEQ ID NO: 165.

147. The bispecific antibody of claim 145 or claim 146, wherein the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 187; and
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 188.

148. The bispecific antibody of any one of claims 145-147, wherein:
(a) the first heavy chain polypeptide comprises the amino acid sequence of SEQ ID NO: 187; and
(b) the second heavy chain polypeptide comprises the amino acid sequence of SEQ ID NO: 188.

149. A bispecific antibody, comprising:
(a) a first heavy chain polypeptide comprising from N- to C-terminal order: a VH region comprising the sequence set forth in SEQ ID NO: 88, a linker of SEQ ID NO: 162, a VL region comprising the sequence set forth in SEQ ID NO: 89, and a Fc region comprising the sequence set forth in SEQ ID NO: 166; and
(b) a second heavy chain polypeptide comprising from N- to C-terminal order: a Fab comprising a V_{H} region comprising the sequence set forth in SEQ ID NO: 70 and an IgG1 CH1 of SEQ ID NO: 169, and a Fc region comprising the sequence set forth in SEQ ID NO:165; and
(c) a light chain comprises a VL region comprising the sequence set forth in SEQ ID NO: 43 and a CL comprising the sequence set forth in SEQ ID NO: 171.

150. The bispecific antibody of claim 149, wherein:
the first heavy chain polypeptide comprises from N-to C-terminal order the scFv comprising the sequence set forth in SEQ ID NO:248 and the Fc region comprising the sequence set forth in SEQ ID NO:166.

151. The bispecific antibody of claim 149 and 150, wherein the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 189;
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 190; and
(c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 191.

152. The bispecific antibody of any one of claims 149-151, wherein:
(a) the first heavy chain polypeptide comprises the amino acid sequence of SEQ ID NO: 189;
(b) the second heavy chain polypeptide comprises the amino acid sequence of SEQ ID NO: 190; and
(c) the light chain comprises the amino acid sequence of SEQ ID NO: 191.

153. A bispecific antibody, comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 168; and
(b) a second heavy chain polypeptide comprising from N- to C-terminal order: a Fab comprising a VH region comprising the sequence set forth in SEQ ID NO: 68 and an IgG1 CH1 of SEQ ID NO: 170, and a Fc region comprising the sequence set forth in SEQ ID NO:167; and
(c) a light chain comprises a VL region comprising the sequence set forth in SEQ ID NO: 39 and a CL comprising the sequence set forth in SEQ ID NO: 171.

154. The bispecific antibody of claim 153, wherein:
the first heavy chain polypeptide comprises from N- to C-terminal order the scFv comprising the sequence set forth in SEQ ID NO:245 and the Fc region comprising the sequence set forth in SEQ ID NO:168.

155. The bispecific antibody of claim 153 and 154, wherein the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 234;
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 235; and
(c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 236.

156. The bispecific antibody of any one of claims 153-155, wherein:
(a) the first heavy chain polypeptide comprises the amino acid sequence of SEQ ID NO: 234;
(b) the second heavy chain polypeptide comprises the amino acid sequence of SEQ ID NO: 235; and
(c) the light chain comprises the amino acid sequence of SEQ ID NO: 236.

157. A bispecific antibody, comprising:
(a) a first heavy chain polypeptide comprising from N- to C-terminal order: a VH region comprising the sequence set forth in SEQ ID NO: 12, a linker of SEQ ID NO: 162, a VL region comprising the sequence set forth in SEQ ID NO: 17, and a Fc region comprising the sequence set forth in SEQ ID NO: 168; and
(b) a second heavy chain polypeptide comprising from N- to C-terminal order: a Fab comprising a VH region comprising the sequence set forth in SEQ ID NO: 70 and an IgG1 CH1 of SEQ ID NO: 170, and a Fc region comprising the sequence set forth in SEQ ID NO:167; and
(c) a light chain comprises a VL region comprising the sequence set forth in SEQ ID NO: 43 and a CL comprising the sequence set forth in SEQ ID NO: 171.

158. The bispecific antibody of claim 157, wherein:
the first heavy chain polypeptide comprises from N- to C-terminal order the scFv comprising the sequence set forth in SEQ ID NO:245 and the Fc region comprising the sequence set forth in SEQ ID NO:168.

159. The bispecific antibody of claim 157 and 158, wherein the bispecific antibody comprises:
(a) a first heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 237;
(b) a second heavy chain polypeptide comprising an amino acid sequence having at least at or about 85%, 86%, 87%, 88%, 89%, 90%, 91%, 92%, 93%, 94%, 95%, 96%, 97%, 98% or 99% identity to SEQ ID NO: 238; and
(c) a light chain comprising an amino acid sequence having at least at or about 85%, 86%, 87%, 88%, 89%, 90%, 91%, 92%, 93%, 94%, 95%, 96%, 97%, 98% or 99% identity to SEQ ID NO: 239.

160. The bispecific antibody of any one of claims 157-159, wherein:
(a) the first heavy chain polypeptide comprises the amino acid sequence of SEQ ID NO: 237;
(b) the second heavy chain polypeptide comprises the amino acid sequence of SEQ ID NO: 238; and
(c) the light chain comprises the amino acid sequence of SEQ ID NO: 239.

161. An isolated anti-CD19 antibody, or an antigen-binding fragment thereof, comprising a heavy chain variable (VH) region and a light chain variable (VL) region, wherein:
(a) the VH region comprises a heavy chain complementarity determining region 1 (CDR-H1), a heavy chain complementarity determining region 2 (CDR-H2), and a heavy chain complementarity determining region 3 (CDR-H3), wherein the CDR-H1 comprises the amino acid sequence set forth in any one of SEQ ID NOs: 9, 118, 131, 140, or 146, the CDR-H2 comprises the amino acid sequence set forth in any one of SEQ ID NOs: 10, 119, 132, 141, or 154, and the CDR-H3 comprises the amino acid sequence set forth in any one of SEQ ID NOs: 11, 120, 124, 128, 133, 136, 142, 147, 151, or 155; and
the VL region comprises a light chain complementarity determining region 1 (CDR-L1), a light chain complementarity determining region 2 (CDR-L2), and a light chain complementarity determining region 3 (CDR-L3), wherein the CDR-L1 comprises the amino acid sequence set forth in SEQ ID NOs: 14, 121, 125, 129, 134, 137, 143, 148, 152, or 156, the CDR-L2 comprises the amino acid sequence set forth in any one of SEQ ID NOs: 15, 122, 126, 25, 138, 144, 149, or 157, and the CDR-L3 comprises the amino acid sequence set forth in any one of SEQ ID NOs: 16, 123, 127, 130, 135, 139, 145, 150, 153, or 158; or
(b) the VH region comprises a CDR-H1, a CDR-H2, and a CDR-H3 contained within any one of SEQ ID NOs: 12, 32, 34, 44, 46, 48, 50, 52, 54, 56, 58, 60, 62, or 88, and the VL region comprises a CDR-L1, a CDR-L2), and a CDR-L3 contained within any one of SEQ ID NOs: 17, 33, 35, 45, 47, 49, 51, 53, 55, 57, 59, 61, 63, or 89.

162. The anti-CD19 antibody, or an antigen-binding fragment thereof, of claim 161, wherein:
the CDR-H1 comprises the amino acid sequence set forth in any one of SEQ ID NOs: 9, 118, 131, 140, or 146, the CDR-H2 comprises the amino acid sequence set forth in any one of SEQ ID NOs: 10, 119, 132, 141, or 154, and the CDR-H3 comprises the amino acid sequence set forth in any one of SEQ ID NOs: 11, 120, 124, 128, 133, 136, 142, 147, 151, or 155; and
the CDR-L1 comprises the amino acid sequence set forth in SEQ ID NOs: 14, 121, 125, 129, 134, 137, 143, 148, 152, or 156, the CDR-L2 comprises the amino acid sequence set forth in any one of SEQ ID NOs: 15, 122, 126, 25, 138, 144, 149, or 157, and the CDR-L3 comprises the amino acid sequence set forth in any one of SEQ ID NOs: 16, 123, 127, 130, 135, 139, 145, 150, 153, or 158.

163. The anti-CD19 antibody, or an antigen-binding fragment thereof, of claim 161, wherein:
the VH region comprises a CDR-H1, a CDR-H2, and a CDR-H3 contained within SEQ ID NO: 32, and the VL region comprises a CDR-L1, a CDR-L2, and a CDR-L3 contained within SEQ ID NO: 33;
the VH region comprises a CDR-H1, a CDR-H2, and a CDR-H3 contained within SEQ ID NO: 34, and the VL region comprises a CDR-L1, a CDR-L2, and a CDR-L3 contained within SEQ ID NO: 35;
the VH region comprises a CDR-H1, a CDR-H2, and a CDR-H3 contained within SEQ ID NO: 44, and the VL region comprises a CDR-L1, a CDR-L2, and a CDR-L3 contained within SEQ ID NO: 45;
the VH region comprises a CDR-H1, a CDR-H2, and a CDR-H3 contained within SEQ ID NO: 46, and the VL region comprises a CDR-L1, a CDR-L2, and a CDR-L3 contained within SEQ ID NO: 47;
the VH region comprises a CDR-H1, a CDR-H2, and a CDR-H3 contained within SEQ ID NO: 48, and the VL region comprises a CDR-L1, a CDR-L2, and a CDR-L3 contained within SEQ ID NO: 49;
the VH region comprises a CDR-H1, a CDR-H2, and a CDR-H3 contained within SEQ ID NO: 50, and the VL region comprises a CDR-L1, a CDR-L2, and a CDR-L3 contained within SEQ ID NO: 51;
the VH region comprises a CDR-H1, a CDR-H2, and a CDR-H3 contained within SEQ ID NO: 52, and the VL region comprises a CDR-L1, a CDR-L2, and a CDR-L3 contained within SEQ ID NO: 53;
the VH region comprises a CDR-H1, a CDR-H2, and a CDR-H3 contained within SEQ ID NO: 54, and the VL region comprises a CDR-L1, a CDR-L2, and a CDR-L3 contained within SEQ ID NO: 55;
the VH region comprises a CDR-H1, a CDR-H2, and a CDR-H3 contained within SEQ ID NO: 56, and the VL region comprises a CDR-L1, a CDR-L2, and a CDR-L3 contained within SEQ ID NO: 57;
the VH region comprises a CDR-H1, a CDR-H2, and a CDR-H3 contained within SEQ ID NO: 58, and the VL region comprises a CDR-L1, a CDR-L2, and a CDR-L3 contained within SEQ ID NO: 59;
the VH region comprises a CDR-H1, a CDR-H2, and a CDR-H3 contained within SEQ ID NO: 60, and the VL region comprises a CDR-L1, a CDR-L2, a CDR-L3 contained within SEQ ID NO: 61;
the VH region comprises a CDR-H1, a CDR-H2, and a CDR-H3 contained within SEQ ID NO: 62, and the VL region comprises a CDR-L1, a CDR-L2, and a CDR-L3 contained within SEQ ID NO: 63;
the VH region comprises a CDR-H1, a CDR-H2, and a CDR-H3 contained within SEQ ID NO: 12, and the VL region comprises a CDR-L1, a CDR-L2, and a CDR-L3 contained within SEQ ID NO: 17; or
the VH region comprises a CDR-H1, a CDR-H2, and a CDR-H3 contained within SEQ ID NO: 88, and the VL region comprises a CDR-L1, a CDR-L2, and a CDR-L3 contained within SEQ ID NO: 89.

164. The anti-CD19 antibody, or an antigen-binding fragment thereof, of any of claims 161-163, wherein:
the VH region comprises the CDR-H1, the CDR-H2, and the CDR-H3 comprising the amino acid sequence set forth in SEQ ID NOS: 9, 10, and 11, respectively, and the VL region comprises the CDR-L1, the CDR-L2, and the CDR-L3 comprising the amino acid sequence set forth in SEQ ID NOS: 14, 15, and 16, respectively;
the VH region comprises the CDR-H1, the CDR-H2, and the CDR-H3 comprising the amino acid sequence set forth in SEQ ID NOS: 118, 119, and 120, respectively, and the VL region comprises the CDR-L1, the CDR-L2, and the CDR-L3 comprising the amino acid sequence set forth in SEQ ID NOS: 121, 122, and 123, respectively;
the VH region comprises the CDR-H1, the CDR-H2, and the CDR-H3 comprising the amino acid sequence set forth in SEQ ID NOS:118, 119, and 124, respectively, and the VL region comprises the CDR-L1, the CDR-L2, and the CDR-L3 comprising the amino acid sequence set forth in SEQ ID NOS: 125, 126, and 127, respectively;
the VH region comprises the CDR-H1, the CDR-H2, and the CDR-H3 comprising the amino acid sequence set forth in SEQ ID NOS:118, 119, and 128, respectively, and the VL region comprises the CDR-L1, the CDR-L2, and the CDR-L3 comprising the amino acid sequence set forth in SEQ ID NOS: 129, 126, and 130, respectively;
the VH region comprises the CDR-H1, the CDR-H2, and the CDR-H3 comprising the amino acid sequence set forth in SEQ ID NOS: 131, 132, and 133, respectively, and the VL region comprises the CDR-L1, the CDR-L2, and the CDR-L3 comprising the amino acid sequence set forth in SEQ ID NOS: 134, 25, and 135, respectively;
the VH region comprises the CDR-H1, the CDR-H2, and the CDR-H3 comprising the amino acid sequence set forth in SEQ ID NOS: 118, 119, and 136, respectively, and the VL region comprises the CDR-L1, the CDR-L2, and the CDR-L3 comprising the amino acid sequence set forth in SEQ ID NOS: 137, 138, and 139, respectively;
the VH region comprises the CDR-H1, the CDR-H2, and the CDR-H3 comprising the amino acid sequence set forth in SEQ ID NOS:140, 141, and 142, respectively, and the VL region comprises the CDR-L1, the CDR-L2, and the CDR-L3 comprising the amino acid sequence set forth in SEQ ID NOS: 143, 144, and 145, respectively;
the VH region comprises the CDR-H1, the CDR-H2, and the CDR-H3 comprising the amino acid sequence set forth in SEQ ID NOS: 146, 119, and 147, respectively, and the VL region comprises the CDR-L1, the CDR-L2, and the CDR-L3 comprising the amino acid sequence set forth in SEQ ID NOS: 148, 149, and 150, respectively;
the VH region comprises the CDR-H1, the CDR-H2, and the CDR-H3 comprising the amino acid sequence set forth in SEQ ID NOS: 118, 119, and 151, respectively, and the VL region comprises the CDR-L1, the CDR-L2, and the CDR-L3 comprising the amino acid sequence set forth in SEQ ID NOS: 152, 126, and 153, respectively; or
the VH region comprises the CDR-H1, the CDR-H2, and the CDR-H3 comprising the amino acid sequence set forth in SEQ ID NOS: 131, 154, and 155, respectively, and the VL region comprises the CDR-L1, the CDR-L2, and the CDR-L3 comprising the amino acid sequence set forth in SEQ ID NOS: 156, 157, and 158, respectively.

165. The anti-CD19 antibody, or an antigen-binding fragment thereof, of any of claims 161-164, wherein:
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 32, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 33;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 34, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 35;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 44, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 45;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 46, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 47;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 48, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 49;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 50, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 51;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 52, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 53;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 54, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 55;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 56, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 57;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 58, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 59;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 60, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 61;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 62, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 63;
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 12, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 17; or
the VH region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 88, and the VL region comprises an amino acid sequence that has at least at or about 85%, 86%, 87%, 88%, 89%, 90%, 91%, 92%, 93%, 94%, 95%, 96%, 97%, 98% or 99% sequence identity to SEQ ID NO: 89.

166. The anti-CD19 antibody, or an antigen-binding fragment thereof, of any of claims 161-165, wherein:
the VH region and the VL region comprise the amino acid sequences set forth in SEQ ID NO: 32 and 33, respectively;
the VH region and the VL region comprise the amino acid sequences set forth in SEQ ID NO: 34 and 35, respectively;
the VH region and the VL region comprise the amino acid sequences set forth in SEQ ID NO: 44 and 45, respectively;
the VH region and the VL region comprise the amino acid sequences set forth in SEQ ID NO: 46 and 47, respectively;
the VH region and the VL region comprise the amino acid sequences set forth in SEQ ID NO: 48 and 49, respectively;
the VH region and the VL region comprise the amino acid sequences set forth in SEQ ID NO: 50 and 51, respectively;
the VH region and the VL region comprise the amino acid sequences set forth in SEQ ID NO: 52 and 53, respectively;
the VH region and the VL region comprise the amino acid sequences set forth in SEQ ID NO: 54 and 55, respectively;
the VH region and the VL region comprise the amino acid sequences set forth in SEQ ID NO: 56 and 57, respectively;
the VH region and the VL region comprise the amino acid sequences set forth in SEQ ID NO: 58 and 59, respectively;
the VH region and the VL region comprise the amino acid sequences set forth in SEQ ID NO: 60 and 61, respectively;
the VH region and the VL region comprise the amino acid sequences set forth in SEQ ID NO: 62 and 63, respectively;
the VH region and the VL region comprise the amino acid sequences set forth in SEQ ID NO: 12 and 17, respectively; or
the VH region and the VL region comprise the amino acid sequences set forth in SEQ ID NO: 88 and 89, respectively.

167. The anti-CD19 antibody, or an antigen-binding fragment thereof, of any of claims 161-166, wherein the VH region comprises the CDR-H1, a CDR-H2, and a CDR-H3 comprising the amino acid sequence set forth in SEQ ID NOs: 9, 10, and 11, respectively, and the VL region comprises a CDR-L1, a CDR-L2, and a CDR-L3 comprising the amino acid sequence set forth in SEQ ID NOS: 14, 15, and 16, respectively.

168. The anti-CD19 antibody, or an antigen-binding fragment thereof, of claim 167, wherein the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 12, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 17.

169. The anti-CD19 antibody, or an antigen-binding fragment thereof, of claim 167 or claim 168, wherein the VH region and the VL region comprises the amino acid sequence set forth in SEQ ID NOs: 12 and 17, respectively.

170. The anti-CD19 antibody, or an antigen-binding fragment thereof, of any of claims 161-166, wherein the VH region comprises the CDR-H1, a CDR-H2, and a CDR-H3 comprising the sequence set forth in SEQ ID NOs: 118, 119, and 120, respectively, and the VL region comprises a CDR-L1, a CDR-L2, and a CDR-L3 comprising the sequence set forth in SEQ ID NOS: 121, 122, 123, respectively.

171. The anti-CD19 antibody, or an antigen-binding fragment thereof, of claim 170, wherein the VH region is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 88, and the VL region that is or comprises an amino acid sequence having at least at or about 85%, 86%, 87%, 88%, 89%, 90%, 91%, 92%, 93%, 94%, 95%, 96%, 97%, 98% or 99% identity to SEQ ID NO: 89.

172. The anti-CD19 antibody, or an antigen-binding fragment thereof, of claim 170 or claim 171, wherein the VH region and the VL region comprises the sequence or are the sequence set forth in SEQ ID NOs: 88 and 89, respectively.

173. The anti-CD19 antibody, or an antigen binding fragment thereof, of any one of claims 161-172 that is a full-length antibody.

174. The anti-CD19 antibody, or an antigen binding fragment thereof, of any one of claims 161-173, wherein the antibody is a human IgG.

175. The anti-CD19 antibody, or an antigen binding fragment thereof, of claim 174, wherein the antibody is IgG1.

176. The anti-CD19 antibody, or an antigen binding fragment thereof, of any one of claims 161-175, wherein the antibody comprises a kappa light chain constant region.

177. The anti-CD19 antibody, or an antigen binding fragment thereof, of any one of claims 161-176, wherein the antibody comprises:
(a) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 254; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 255;
(b) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 256; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 257;
(c) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 258; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 259;
(d) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 260; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 261;
(e) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 262; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 263;
(f) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 264; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 265;
(g) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 266; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 267;
(h) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 268; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 269;
(i) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 270; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 271;
(j) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 272; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 273;
(k) (i) a heavy chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 274; and (ii) a light chain comprising an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 275.

178. The anti-CD19 antibody, or an antigen binding fragment thereof, of any one of claims 161-177, wherein the antibody comprises:
(a) (i) a heavy chain comprising the amino acid sequence of SEQ ID NO: 254; and (ii) a light chain comprising the amino acid sequence of SEQ ID NO: 255;
(b) (i) a heavy chain comprising the amino acid sequence of SEQ ID NO: 256; and (ii) a light chain comprising the amino acid sequence of SEQ ID NO: 257;
(c) (i) a heavy chain comprising the amino acid sequence of SEQ ID NO: 258; and (ii) a light chain comprising the amino acid sequence of SEQ ID NO: 259;
(d) (i) a heavy chain comprising the amino acid sequence of SEQ ID NO: 260; and (ii) a light chain comprising the amino acid sequence of SEQ ID NO: 261;
(e) (i) a heavy chain comprising the amino acid sequence of SEQ ID NO: 262; and (ii) a light chain comprising the amino acid sequence of SEQ ID NO: 263;
(f) (i) a heavy chain comprising the amino acid sequence of SEQ ID NO: 264; and (ii) a light chain comprising the amino acid sequence of SEQ ID NO: 265;
(g) (i) a heavy chain comprising the amino acid sequence of SEQ ID NO: 266; and (ii) a light chain comprising the amino acid sequence of SEQ ID NO: 267;
(h) (i) a heavy chain comprising the amino acid sequence of SEQ ID NO: 268; and (ii) a light chain comprising the amino acid sequence of SEQ ID NO: 269;
(i) (i) a heavy chain comprising the amino acid sequence of SEQ ID NO: 270; and (ii) a light chain comprising the amino acid sequence of SEQ ID NO: 271;
(j) (i) a heavy chain comprising the amino acid sequence of SEQ ID NO: 272; and (ii) a light chain comprising the amino acid sequence of SEQ ID NO: 273; or
(k) (i) a heavy chain comprising the amino acid sequence of SEQ ID NO: 274; and (ii) a light chain comprising the amino acid sequence of SEQ ID NO: 275.

179. The anti-CD19 antibody, or an antigen binding fragment thereof, of any one of claims 161-172, that is an antigen-binding fragment.

180. The anti-CD19 antibody, or an antigen binding fragment thereof, of claim 179, wherein the antigen-binding fragment is selected from the group consisting of Fab, Fab′, F(ab′)2, Fv, and scFv.

181. The anti-CD19 antibody, or an antigen binding fragment thereof, of claim 179 or claim 180, wherein the antigen-binding fragment is an scFv.

182. The anti-CD19 antibody, or an antigen binding fragment thereof, of claim 181,
wherein the scFv comprises a VH-linker-VL arrangement.

183. The anti-CD19 antibody, or an antigen binding fragment thereof, of claim 181,
wherein the scFv comprises a VL-linker-VH arrangement.

184. The anti-CD19 antibody, or an antigen binding fragment thereof, of any one of claim 182 or 183, wherein the linker is a peptide linker comprising (Gly4Ser)3 (SEQ ID NO: 162).

185. The anti-CD19 antibody, or an antigen binding fragment thereof, of any one of claims 181-184, wherein the scFv comprises:
(a) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 245;
(b) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 248;
(c) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 249; or
(d) an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 252.

186. The anti-CD19 antibody, or an antigen binding fragment thereof, of any one of claims 181-185, wherein the scFv comprises:
(a) the amino acid sequence of SEQ ID NO: 245;
(b) the amino acid sequence of SEQ ID NO: 248;
(c) the amino acid sequence of SEQ ID NO: 249; or
(d) the amino acid sequence of SEQ ID NO: 252.

187. The anti-CD19 antibody, or an antigen binding fragment thereof, of any one of claims 181-186, wherein the scFv comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 245.

188. The anti-CD19 antibody, or an antigen binding fragment thereof, of any one of claims 181-187, wherein the scFv comprises the amino acid sequence of SEQ ID NO: 245.

189. The anti-CD19 antibody, or an antigen binding fragment thereof, of any one of claims 181-186, wherein the scFv comprises an amino acid sequence that has at least at or about 85%, 86%, 87%, 88%, 89%, 90%, 91%, 92%, 93%, 94%, 95%, 96%, 97%, 98% or 99% sequence identity to the amino acid sequence of SEQ ID NO: 248.

190. The anti-CD19 antibody, or an antigen binding fragment thereof, of any one of claims 181-186 or 189, wherein the scFv comprises the amino acid sequence of SEQ ID NO: 248.

191. A polynucleotide comprising a nucleic acid sequence(s) encoding the bispecific antibody of any of claims 1-160 or the anti-CD19 antibody or an antigen-binding fragment thereof of any one of claims 161-190.

192. A polynucleotide system comprising a nucleic acid sequence encoding each chain of the bispecific antibody of any one of claims 1-160 or the anti-CD19 antibody or an antigen-binding fragment thereof of any one of claims 161-190.

193. A polynucleotide system comprising (a) a first nucleic acid sequence encoding the first heavy chain of the bispecific antibody of any one of claims 1-160, and (b) a second nucleic acid sequence encoding the second heavy chain of the bispecific antibody of any one of claims 1-160.

194. A vector comprising the polynucleotide of claim 191.

195. The vector of claim 194, wherein the vector is an expression vector.

196. A vector system comprising a plurality of vectors, each vector comprising a nucleic acid sequence of the polynucleotide system of claim 192 or claim 193.

197. The vector system of claim 196, wherein each vector is an expression vector.

198. A cell comprising the polynucleotide of claim 191 or the vector of any of claims 194-195.

199. A cell comprising the polynucleotide system of claim 192 or claim 193 or the vector system of claim 196 or claim 197.

200. The cell of claim 198 or claim 199, wherein the cell is a mammalian cell.

201. A method of producing an antibody comprising culturing the cell of any one of claims 198-200 under a condition that produces the antibody.

202. The method of claim 201, further comprising recovering the antibody produced by the cell.

203. An antibody or antigen-binding fragment thereof or bispecific antibody produced by the method of claim 201 or claim 202.

204. A composition comprising the bispecific antibody of any of claims 1-160.

205. A composition comprising the anti-CD19 antibody or an antigen-binding fragment thereof of any of claims 161-190.

206. The composition of claim 204 or claim 205, further comprising a pharmaceutically acceptable excipient.

207. A method for treating a disease or condition in a subject in need thereof, the method comprising administering a therapeutically effective amount of the bispecific antibody of any of claims 1-160 or composition of claim 204 or claim 206.

208. A method for treating a disease or condition in a subject in need thereof, the method comprising administering a therapeutically effective amount of the anti-CD19 antibody or an antigen-binding fragment thereof of any of claims 161-190 or composition of claim 205 or claim 206.

209. The method of claim 207 or claim 208, wherein the disease or condition is an infectious disease or disorder, an autoimmune disease, an inflammatory disease, or a tumor or a cancer.

210. The method of any one of claims 207-209, wherein the disease or condition is associated with overexpression of B cells in tissues.

211. The method of any of claims 207-210, wherein the disease or condition is an autoimmune disorder.

212. The method of claim 211, wherein the autoimmune disorder is associated with presence of increased autoantibodies and/or autoreactive B cells in the subject compared to a healthy subject.

213. The method of claim 211 or claim 212, wherein the autoimmune disorder is selected from the group consisting of psoriasis, psoriatic arthritis, Type 1 diabetes mellitus (Type 1 diabetes), rheumatoid arthritis (RA), systemic lupus erythematosus (SLE), cutaneous lupus erythematosus (CLE), multiple sclerosis (MS), inflammatory bowel disease (IBD), lymphocytic colitis, acute disseminated encephalomyelitis (ADEM), Addison's disease, alopecia universalis, ankylosing spondylitisis, antiphospholipid antibody syndrome (APS), aplastic anemia, autoimmune hemolytic anemia, autoimmune hepatitis, autoimmune inner ear disease (AIED), autoimmune lymphoproliferative syndrome (ALPS), autoimmune oophoritis, Balo disease, Behcet's disease, bullous pemphigoid, cardiomyopathy, Chagas' disease, chronic fatigue immune dysfunction syndrome (CFIDS), chronic inflammatory demyelinating polyneuropathy, Crohn's disease, cicatrical pemphigoid, coeliac sprue-dermatitis herpetiformis, cold agglutinin disease, CREST syndrome, Degos disease, discoid lupus erythematosus (DLE), dysautonomia, endometriosis, essential mixed cryoglobulinemia, fibromyalgia-fibromyositis, Goodpasture's syndrome, Grave's disease, Guillain-Barre syndrome (GBS), Hashimoto's thyroiditis, Hidradenitis suppurativa, idiopathic and/or acute thrombocytopenic purpura, idiopathic pulmonary fibrosis, IgA neuropathy, interstitial cystitis, juvenile arthritis, Kawasaki's disease, lichen planus, Lyme disease, Meniere disease, mixed connective tissue disease (MCTD), myasthenia gravis, neuromyotonia, opsoclonus myoclonus syndrome (OMS), optic neuritis, Ord's thyroiditis, pemphigus vulgaris, pernicious anemia, polychondritis, polymyositis and dermatomyositis, primary biliary cirrhosis, polyarteritis nodosa, polyglandular syndromes, polymyalgia rheumatica, primary agammaglobulinemia, Raynaud phenomenon, Reiter's syndrome, rheumatic fever, sarcoidosis, scleroderma, Sjögren's syndrome, stiff person syndrome, Takayasu's arteritis, temporal arteritis (also known as “giant cell arteritis”), ulcerative colitis, collagenous colitis, uveitis, vasculitis, vitiligo, vulvodynia (“vulvar vestibulitis”), and Wegener's granulomatosis.

214. The method of any of claims 211-213, wherein the autoimmune disorder is systemic lupus erythematosus (SLE), rheumatoid arthritis, Sjogren disease, or systemic sclerosis.

215. The method of any of claims 211-214, wherein the autoimmune disorder is SLE.

216. The method of any of claims 207-210, wherein the disease or condition is a cancer.

217. The method of claim 209 or claim 216, wherein the cancer is a solid tumor.

218. The method of claim 209, 216 or 217, wherein the cancer is a melanoma, breast carcinoma, HCC, Renal cell carcinoma, Colorectal carcinoma, non-small-cell lung carcinoma, Cervical cancer or Mesothelioma.

219. The method of claim 209, 216 or 217, wherein the cancer is a B-cell-related cancer selected from the group consisting of multiple myeloma, malignant plasma cell neoplasm, plasma cell leukemia, plasmacytoma, B-cell prolymphocytic leukemia, hairy cell leukemia, B-cell non-Hodgkin's lymphoma (NHL), chronic lymphocytic leukemia (CLL), follicular lymphoma, Burkitt's lymphoma, marginal zone lymphoma, mantle cell lymphoma, large cell lymphoma, precursor B-lymphoblastic lymphoma, Waldenstrom's macroglobulinemia, diffuse large B-cell lymphoma (DLBCL), mucosa-associated lymphatic tissue lymphoma (MALT lymphoma), small cell lymphocytic lymphoma, Primary mediastinal (thymic) large B-cell lymphoma, lymphoplasmacytic lymphoma, nodal marginal zone B cell lymphoma, splenic marginal zone lymphoma, intravascular large B-cell lymphoma, primary effusion lymphoma, lymphomatoid granulomatosis, T cell/histiocyte-rich large B-cell lymphoma, primary CNS (central nervous system) lymphoma, primary cutaneous diffuse large B-cell lymphoma (leg type), EBV positive diffuse large B-cell lymphoma of the elderly, diffuse large B-cell lymphoma associated with inflammation, ALK-positive large B-cell lymphoma, and plasmablastic lymphoma.

220. The method of any of claims 207-210, wherein the disease or condition is an inflammatory disease.

221. The method of claim 209 or 219, wherein the inflammatory disease is selected from the group consisting of asthma, atopic dermatitis (eczema), contact dermatitis and gout.

222. A kit comprising the bispecific antibody of any of claims 1-160 or the anti-CD19 antibody or an antigen-binding fragment thereof of any of claims 161-190 and, optionally, instructions for use.
